(19) United States
(12) Patent Application Publication
(10) Pub. No.: US 2007/0224201 A1
(43) Pub. Date: Sep. 27, 2007

(54) Compositions and methods for the diagnosis and treatment of tumor

(75) Inventors: Thomas D. Wu, San Francisco, CA (US); Zemin Zhang, Foster City, CA (US); Yan Zhou, South Pasadena, CA (US)

Correspondence Address: GENENTECH, INC., 1 DNA WAY, SOUTH SAN FRANCISCO, CA, 94080, US

(73) Assignee: Genentech, Inc., South San Francisco, CA (US)

(21) Appl. No.: 10/529,351

(22) PCT Filed: Sep. 29, 2003
(86) PCT No.: PCT/US03/28547

§ 371 Date: May 24, 2006

Related U.S. Application Data
(60) Provisional application No. 60/414,971, filed on Oct. 2, 2002.

(51) Int. Cl. C12Q 1/68; G01N 33/574; C07H 21/04; C12P 21/06; A61K 39/395; C07K 14/82; C07K 16/30; C07K 16/46

(52) U.S. Cl. 424/155.1; 536/23.5; 435/6; 435/7.23; 435/69.1; 435/320.1; 435/325; 530/350; 530/388.8; 530/391.1; 424/178.1; 435/252.3

(57) ABSTRACT

The present invention is directed to compositions of matter useful for the diagnosis and treatment of tumor in mammals and to methods of using those compositions of matter for the same.

### FIELD OF THE INVENTION

The present invention is directed to compositions of matter useful for the diagnosis and treatment of tumor in mammals and to methods of using those compositions of matter for the same.

### BACKGROUND OF THE INVENTION

Malignant tumors (cancers) are the second leading cause of death in the United States, after heart disease (Boring et al., CA Cancel J. Clin. 43:7 (1993)). Cancer is characterized by the increase in the number of abnormal, or neoplastic, cells derived from a normal tissue which proliferate to form a tumor mass, the invasion of adjacent tissues by these neoplastic tumor cells, and the generation of malignant cells which eventually spread via the blood or lymphatic system to regional lymph nodes and to distant sites via a process called metastasis. In a cancerous state, a cell proliferates under conditions in which normal cells would not grow. Cancer manifests itself in a wide variety of forms, characterized by different degrees of invasiveness and aggressiveness.

In attempts to discover effective cellular targets for cancer diagnosis and therapy, researchers have sought to identify transmembrane or otherwise membrane-associated polypeptides that are specifically expressed on the surface of one or more particular type(s) of cancer cell as compared to on one or more normal non-cancerous cell(s). Often, such membrane-associated polypeptides are more abundantly expressed on the surface of the cancer cells as compared to on the surface of the non-cancerous cells. The identification of such tumor-associated cell surface antigen polypeptides has given rise to the ability to specifically target cancer cells for destruction via antibody-based therapies. In this regard, it is noted that antibody-based therapy has proved very effective in the treatment of certain cancers. For example, HERCEPTIN® and RITUXAN® (both from Genentech Inc., South San Francisco, Calif.) are antibodies that have been used successfully to treat breast cancer and non-Hodgkin's lymphoma, respectively. More specifically, HERCEPTIN® is a recombinant DNA-derived humanized monoclonal antibody that selectively binds to the extracellular domain of the human epidermal growth factor receptor 2 (HER2) proto-oncogene. HER2 protein overexpression is observed in 25-30% of primary breast cancers. RITUXAN® is a genetically engineered chimeric murine/human monoclonal antibody directed against the CD20 antigen found on the surface of normal and malignant B lymphocytes. Both these antibodies are recombinantly produced in CHO cells.

In other attempts to discover effective cellular targets for cancer diagnosis and therapy, researchers have sought to identify (1) non-membrane-associated polypeptides that are specifically produced by one or more particular type(s) of cancer cell(s) as compared to by one or more particular type(s) of non-cancerous normal cell(s), (2) polypeptides that are produced by cancer cells at an expression level that is significantly higher than that of one or more normal non-cancerous cell(s), or (3) polypeptides whose expression is specifically limited to only a single (or very limited number of different) tissue type(s) in both the cancerous and non-cancerous state (e.g., normal prostate and prostate tumor tissue). Such polypeptides may remain intracellularly located or may be secreted by the cancer cell. Moreover, such polypeptides may be expressed not by the cancer cell itself, but rather by cells which produce and/or secrete polypeptides having a potentiating or growth-enhancing effect on cancer cells. Such secreted polypeptides are often proteins that provide cancer cells with a growth advantage over normal cells and include such things as, for example, angiogenic factors, cellular adhesion factors, growth factors, and the like. Identification of antagonists of such non-membrane associated polypeptides would be expected to serve as effective therapeutic agents for the treatment of such cancers. Furthermore, identification of the expression pattern of such polypeptides would be useful for the diagnosis of particular cancers in mammals.

Despite the above identified advances in mammalian cancer therapy, there is a great need for additional diagnostic and therapeutic agents capable of detecting the presence of tumor in a mammal and for effectively inhibiting neoplastic cell growth, respectively. Accordingly, it is an objective of the present invention to identify: (1) cell membrane-associated polypeptides that are more abundantly expressed on one or more type(s) of cancer cell(s) as compared to on normal cells or on other different cancer cells, (2) non-membrane-associated polypeptides that are specifically produced by one or more particular type(s) of cancer cell(s) (or by other cells that produce polypeptides having a potentiating effect on the growth of cancer cells) as compared to by one or more particular type(s) of non-cancerous normal cell(s), (3) non-membrane-associated polypeptides that are produced by cancer cells at an expression level that is significantly higher than that of one or more normal non-cancerous cell(s), or (4) polypeptides whose expression is specifically limited to only a single (or very limited number of different) tissue type(s) in both a cancerous and non-cancerous state (e.g., normal prostate and prostate tumor tissue), and to use those polypeptides, and their encoding nucleic acids, to produce compositions of matter useful in the therapeutic treatment and diagnostic detection of cancer in mammals. It is also an objective of the present invention to identify cell membrane-associated, secreted or intracellular polypeptides whose expression is limited to a single or very limited number of tissues, and to use those polypeptides, and their encoding nucleic acids, to produce compositions of matter useful in the therapeutic treatment and diagnostic detection of cancer in mammals.

### SUMMARY OF THE INVENTION

### A. Embodiments

In the present specification, Applicants describe for the first time the identification of various cellular polypeptides (and their encoding nucleic acids or fragments thereof) which are expressed to a greater degree on the surface of or by one or more types of cancer cell(s) as compared to on the surface of or by one or more types of normal non-cancer cells. Alternatively, such polypeptides are expressed by cells which produce and/or secrete polypeptides having a potentiating or growth-enhancing effect on cancer cells. Again alternatively, such polypeptides may not be overexpressed by tumor cells as compared to normal cells of the same tissue type, but rather may be specifically expressed by both tumor cells and normal cells of only a single or very limited number of tissue types (preferably tissues which are not essential for life, e.g., prostate, etc.). All of the above polypeptides are herein referred to as Tumor-associated Antigenic Target polypeptides (“TAT” polypeptides) and are expected to serve as effective targets for cancer therapy and diagnosis in mammals.

Accordingly, in one embodiment of the present invention, the invention provides an isolated nucleic acid molecule having a nucleotide sequence that encodes a tumor-associated antigenic target polypeptide or fragment thereof (a “TAT” polypeptide).

In certain aspects, the isolated nucleic acid molecule comprises a nucleotide sequence having at least about 80% nucleic acid sequence identity, alternatively at least about 81%, 82%, 83%, 84%, 85%, 86%, 87%, 88%, 89%, 90%, 91%, 92%, 93%, 94%, 95%, 96%, 97%, 98%, 99% or 100% nucleic acid sequence identity, to (a) a DNA molecule encoding a full-length TAT polypeptide having an amino acid sequence as disclosed herein, a TAT polypeptide amino acid sequence lacking the signal peptide as disclosed herein, an extracellular domain of a transmembrane TAT polypeptide, with or without the signal peptide, as disclosed herein or any t other specifically defined fragment of a full-length TAT polypeptide amino acid sequence as disclosed herein, or (b) the complement of the DNA molecule of (a).

In other aspects, the isolated nucleic acid molecule comprises a nucleotide sequence having at least about 80% nucleic acid sequence identity, alternatively at least about 81%, 82%, 83%, 84%, 85%, 86%, 87%, 88%, 89%, 90%, 91%, 92%, 93%, 94%, 95%, 96%, 97%, 98%, 99% or 100% nucleic acid sequence identity, to (a) a DNA molecule comprising the coding sequence of a full-length TAT polypeptide cDNA as disclosed herein, the coding sequence of a TAT polypeptide lacking the signal peptide as disclosed herein, the coding sequence of an extracellular domain of a transmembrane TAT polypeptide, with or without the signal peptide, as disclosed herein or the coding sequence of any other specifically defined fragment of the full-length TAT polypeptide amino acid sequence as disclosed herein, or (b) the complement of the DNA molecule of (a).

In further aspects, the invention concerns an isolated nucleic acid molecule comprising a nucleotide sequence having at least about 80% nucleic acid sequence identity, alternatively at least about 81%, 82%, 83%, 84%, 85%, 86%, 87%, 88%, 89%, 90%, 91%, 92%, 93%, 94%, 95%, 96%, 97%, 98%, 99% or 100% nucleic acid sequence identity, to (a) a DNA molecule that encodes the same mature polypeptide encoded by the full-length coding region of any of the human protein cDNAs deposited with the ATCC as disclosed herein, or (b) the complement of the DNA molecule of (a).

Another aspect of the invention provides an isolated nucleic acid molecule comprising a nucleotide sequence encoding a TAT polypeptide which is either transmembrane domain-deleted or transmembrane domain-inactivated, or is complementary to such encoding nucleotide sequence, wherein the transmembrane domain(s) of such polypeptide(s) are disclosed herein. Therefore, soluble extracellular domains of the herein described TAT polypeptides are contemplated.

In other aspects, the present invention is directed to isolated nucleic acid molecules which hybridize to (a) a nucleotide sequence encoding a TAT polypeptide having a full-length amino acid sequence as disclosed herein, a TAT polypeptide amino acid sequence lacking the signal peptide as disclosed herein, an extracellular domain of a transmembrane TAT polypeptide, with or without the signal peptide, as disclosed herein or any other specifically defined fragment of a full-length TAT polypeptide amino acid sequence as disclosed herein, or (b) the complement of the nucleotide sequence of (a). In this regard, an embodiment of the present invention is directed to fragments of a full-length TAT polypeptide coding sequence, or the complement thereof, as disclosed herein, that may find use as, for example, hybridization probes useful as, for example, diagnostic probes, antisense oligonucleotide probes, or for encoding fragments of a full-length TAT polypeptide that may optionally encode a polypeptide comprising a binding site for an anti-TAT polypeptide antibody, a TAT binding oligopeptide or other small organic molecule that binds to a TAT polypeptide. Such nucleic acid fragments are usually at least about S nucleotides in length, alternatively at least about 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 nucleotides in length, wherein in this context the term “about” means the referenced nucleotide sequence length plus or minus 10% of that referenced length. It is noted that novel fragments of a TAT polypeptide-encoding nucleotide sequence may be determined in a routine manner by aligning the TAT polypeptide-encoding nucleotide sequence with other known nucleotide sequences using any of a number of well known sequence alignment programs and determining which TAT polypeptide-encoding nucleotide sequence fragment(s) are novel. All of such novel fragments of TAT polypeptide-encoding nucleotide sequences are contemplated herein. Also contemplated are the TAT polypeptide fragments encoded by these nucleotide molecule fragments, preferably those TAT polypeptide fragments that comprise a binding site for an anti-TAT antibody, a TAT binding oligopeptide or other small organic molecule that binds to a TAT polypeptide.

In another embodiment, the invention provides isolated TAT polypeptides encoded by any of the isolated nucleic acid sequences hereinabove identified.

In a certain aspect, the invention concerns an isolated TAT polypeptide, comprising an amino acid sequence having at least about 80% amino acid sequence identity, alternatively at least about 81%, 82%, 83%, 84%, 85%, 86%, 87%, 88%, 89%, 90%, 91%, 92%, 93%, 94%, 95%, 96%, 97%, 98%, 99% or 100% amino acid sequence identity, to a TAT polypeptide having a full-length amino acid sequence as disclosed herein, a TAT polypeptide amino acid sequence lacking the signal peptide as disclosed herein, an extracellular domain of a transmembrane TAT polypeptide protein, with or without the signal peptide, as disclosed herein, an amino acid sequence encoded by any of the nucleic acid sequences disclosed herein or any other specifically defined fragment of a full-length TAT polypeptide amino acid sequence as disclosed herein.

In a further aspect, the invention concerns an isolated TAT polypeptide comprising an amino acid sequence having at least about 80% amino acid sequence identity, alternatively at least about 81%, 82%, 83%, 84%, 85%, 86%, 87%, 88%, 89%, 90%, 91%, 92%, 93%, 94%, 95%, 96%, 97%, 98%, or 99% amino acid sequence identity, to an amino acid sequence encoded by any of the human protein cDNAs deposited with the ATCC as disclosed herein.

In a specific aspect, the invention provides an isolated TAT polypeptide without the N-terminal signal sequence and/or without the initiating methionine and is encoded by a nucleotide sequence that encodes such an amino acid sequence as hereinbefore described. Processes for producing the same are also herein described, wherein those processes comprise culturing a host cell comprising a vector which comprises the appropriate encoding nucleic acid molecule under conditions suitable for expression of the TAT polypeptide and recovering the TAT polypeptide from the cell culture.

Another aspect of the invention provides an isolated TAT polypeptide which is either transmembrane domain-deleted or transmembrane domain-inactivated. Processes for producing the same are also herein described, wherein those processes comprise culturing a host cell comprising a vector which comprises the appropriate encoding nucleic acid molecule under conditions suitable for expression of the TAT polypeptide and recovering the TAT polypeptide from the cell culture.

In other embodiments of the present invention, the invention provides vectors comprising DNA encoding any of the herein described polypeptides. Host cells comprising any such vector are also provided. By way of example, the host cells may be CHO cells, E. coli cells, or yeast cells. A process for producing any of the herein described polypeptides is further provided and comprises culturing host cells under conditions suitable for expression of the desired polypeptide and recovering the desired polypeptide from the cell culture.

In other embodiments, the invention provides isolated chimeric polypeptides comprising any of the herein described TAT polypeptides fused to a heterologous (non-TAT) polypeptide. Example of such chimeric molecules comprise any of the herein described TAT polypeptides fused to a heterologous polypeptide such as, for example, an epitope tag sequence or a Fc region of an immunoglobulin.

In another embodiment, the invention provides an antibody which binds, preferably specifically, to any of the above or below described polypeptides. Optionally, the antibody is a monoclonal antibody, antibody fragment, chimeric antibody, humanized antibody, single-chain antibody or antibody that competitively inhibits the binding of an anti-TAT polypeptide antibody to its respective antigenic epitope. Antibodies of the present invention may optionally be conjugated to a growth inhibitory agent or cytotoxic agent such as a toxin, including, for example, a maytansinoid or calicheamicin, an antibiotic, a radioactive isotope, a nucleolytic enzyme, or the like. The antibodies of the present invention may optionally be produced in CHO cells or bacterial cells and preferably induce death of a cell to which they bind. For diagnostic purposes, the antibodies of the present invention may be detectably labeled, attached to a solid support, or the like.

In other embodiments of the present invention, the invention provides vectors comprising DNA encoding any of the herein described antibodies. Host cell comprising any such vector are also provided. By way of example, the host cells may be CHO cells, E. coli cells, or yeast cells. A process for producing any of the herein described antibodies is further provided and comprises culturing host cells under conditions suitable for expression of the desired antibody and recovering the desired antibody from the cell culture.

In another embodiment, the invention provides oligopeptides (“TAT binding oligopeptides”) which bind, preferably specifically, to any of the above or below described TAT polypeptides. Optionally, the TAT binding oligopeptides of the present invention may be conjugated to a growth inhibitory agent or cytotoxic agent such as a toxin, including, for example, a maytansinoid or calicheamicin, an antibiotic, a radioactive isotope, a nucleolytic enzyme, or the like. The TAT binding oligopeptides of the present invention may optionally be produced in CHO cells or bacterial cells and preferably induce death of a cell to which they bind. For diagnostic purposes, the TAT binding oligopeptides of the present invention may be detectably labeled, attached to a solid support, or the like.

In other embodiments of the present invention, the invention provides vectors comprising DNA encoding any of the herein described TAT binding oligopeptides. Host cell comprising any such vector are also provided. By way of example, the host cells may be CHO cells, E. coli cells, or yeast cells. A process for producing any of the herein described TAT binding oligopeptides is further provided and comprises culturing host cells under conditions suitable for expression of the desired oligopeptide and recovering the desired oligopeptide from the cell culture.

In another embodiment, the invention provides small organic molecules (“TAT binding organic molecules”) which bind, preferably specifically, to any of the above or below described TAT polypeptides. Optionally, the TAT binding organic molecules of the present invention may be conjugated to a growth inhibitory agent or cytotoxic agent such as a toxin, including, for example, a maytansinoid or calicheamicin, an antibiotic, a radioactive isotope, a nucleolytic enzyme, or the like. The TAT binding organic molecules of the present invention preferably induce death of a cell to which they bind. For diagnostic purposes, the TAT binding organic molecules of the present invention may be detectably labeled, attached to a solid support, or the like.

In a still further embodiment, the invention concerns a composition of matter comprising a TAT polypeptide as described herein, a chimeric TAT polypeptide as described herein, an anti-TAT antibody as described herein, a TAT binding oligopeptide as described herein, or a TAT binding organic molecule as described herein, in combination with a carrier. Optionally, the carrier is a pharmaceutically acceptable carrier. In yet another embodiment, the invention concerns an article of manufacture comprising a container and a composition of matter contained within the container, wherein the composition of matter may comprise a TAT polypeptide as described herein, a chimeric TAT polypeptide as described herein, an anti-TAT antibody as described herein, a TAT binding oligopeptide as described herein, or a TAT binding organic molecule as described herein. The article may further optionally comprise a label affixed to the container, or a package insert included with the container, that refers to the use of the composition of matter for the therapeutic treatment or diagnostic detection of a tumor.

Another embodiment of the present invention is directed to the use of a TAT polypeptide as described herein, a chimeric TAT polypeptide as described herein, an anti-TAT polypeptide antibody as described herein, a TAT binding oligopeptide as described herein, or a TAT binding organic molecule as described herein, for the preparation of a medicament useful in the treatment of a condition which is responsive to the TAT polypeptide, chimeric TAT polypeptide, anti-TAT polypeptide antibody, TAT binding oligopeptide, or TAT binding organic molecule.

### B. Additional Embodiments

Another embodiment of the present invention is directed to a method for inhibiting the growth of a cell that expresses a TAT polypeptide, wherein the method comprises contacting the cell with an antibody, an oligopeptide or a small organic molecule that binds to the TAT polypeptide, and wherein the binding of the antibody, oligopeptide or organic molecule to the TAT polypeptide causes inhibition of the growth of the cell expressing the TAT polypeptide. In preferred embodiments, the cell is a cancer cell and binding of the antibody, oligopeptide or organic molecule to the TAT polypeptide causes death of the cell expressing the TAT polypeptide. Optionally, the antibody is a monoclonal antibody, antibody fragment, chimeric antibody, humanized antibody, or single-chain antibody. Antibodies, TAT binding oligopeptides and TAT binding organic molecules employed in the methods of the present invention may optionally be conjugated to a growth inhibitory agent or cytotoxic agent such as a toxin, including, for example, a maytansinoid or calicheamicin, an antibiotic, a radioactive isotope, a nucleolytic enzyme, or the like. The antibodies and TAT binding oligopeptides employed in the methods of the present invention may optionally be produced in CHO cells or bacterial cells.

Yet another embodiment of the present invention is directed to a method of therapeutically treating a mammal having a cancerous tumor comprising cells that express a TAT polypeptide, wherein the method comprises administering to the mammal a therapeutically effective amount of an antibody, an oligopeptide or a small organic molecule that binds to the TAT polypeptide, thereby resulting in the effective therapeutic treatment of the tumor. Optionally, the antibody is a monoclonal antibody, antibody fragment, chimeric antibody, humanized antibody, or single-chain antibody. Antibodies, TAT binding oligopeptides and TAT binding organic molecules employed in the methods of the present invention may optionally be conjugated to a growth inhibitory agent or cytotoxic agent such as a toxin, including, for example, a maytansinoid or calicheamicin, an antibiotic, a radioactive isotope, a nucleolytic enzyme, or the like. The antibodies and oligopeptides employed in the methods of the present invention may optionally be produced in CHO cells or bacterial cells.

Yet another embodiment of the present invention is directed to a method of determining the presence of a TAT polypeptide in a sample suspected of containing the TAT polypeptide, wherein the method comprises exposing the sample to an antibody, oligopeptide or small organic molecule that binds to the TAT polypeptide and determining binding of the antibody, oligopeptide or organic molecule to the TAT polypeptide in the sample, wherein the presence of such binding is indicative of the presence of the TAT polypeptide in the sample. Optionally, the sample may contain cells (which may be cancer cells) suspected of expressing the TAT polypeptide. The antibody, TAT binding oligopeptide or TAT binding organic molecule employed in the method may optionally be detectably labeled, attached to a solid support, or the like.

A further embodiment of the present invention is directed to a method of diagnosing the presence of a tumor in a mammal, wherein the method comprises detecting the level of expression of a gene encoding a TAT polypeptide (a) in a test sample of tissue cells obtained from said mammal, and (b) in a control sample of known normal non-cancerous cells of the same tissue origin or type, wherein a higher level of expression of the TAT polypeptide in the test sample, as compared to the control sample, is indicative of the presence of tumor in the mammal from which the test sample was obtained.

Another embodiment of the present invention is directed to a method of diagnosing the presence of a tumor in a mammal, wherein the method comprises (a) contacting a test sample comprising tissue cells obtained from the mammal with an antibody, oligopeptide or small organic molecule that binds to a TAT polypeptide and (b) detecting the formation of a complex between the antibody, oligopeptide or small organic molecule and the TAT polypeptide in the test sample, wherein the formation of a complex is indicative of the presence of a tumor in the mammal. Optionally, the antibody, TAT binding oligopeptide or TAT binding organic molecule employed is detectably labeled, attached to a solid support, or the like, and/or the test sample of tissue cells is obtained from an individual suspected of having a cancerous tumor.

Yet another embodiment of the present invention is directed to a method for treating or preventing a cell proliferative disorder associated with altered, preferably increased, expression or activity of a TAT polypeptide, the method comprising administering to a subject in need of such treatment an effective amount of an antagonist of a TAT polypeptide. Preferably, the cell proliferative disorder is cancer and the antagonist of the TAT polypeptide is an anti-TAT polypeptide antibody, TAT binding oligopeptide, TAT binding organic molecule or antisense oligonucleotide. Effective treatment or prevention of the cell proliferative disorder may be a result of direct killing or growth inhibition of cells that express a TAT polypeptide or by antagonizing the cell growth potentiating activity of a TAT polypeptide.

Yet another embodiment of the present invention is directed to a method of binding an antibody, oligopeptide or small organic molecule to a cell that expresses a TAT polypeptide, wherein the method comprises contacting a cell that expresses a TAT polypeptide with said antibody, oligopeptide or small organic molecule under conditions which are suitable for binding of the antibody, oligopeptide or small organic molecule to said TAT polypeptide and allowing binding therebetween.

Other embodiments of the present invention are directed to the use of (a) a TAT polypeptide, (b) a nucleic acid encoding a TAT polypeptide or a vector or host cell comprising that nucleic acid, (c) an anti-TAT polypeptide antibody, (d) a TAT-binding oligopeptide, or (e) a TAT-binding small organic molecule in the preparation of a medicament useful for (i) the therapeutic treatment or diagnostic detection of a cancer or tumor, or (ii) the therapeutic treatment or prevention of a cell proliferative disorder.

Another embodiment of the present invention is directed to a method for inhibiting the growth of a cancer cell, wherein the growth of said cancer cell is at least in part dependent upon the growth potentiating effect(s) of a TAT polypeptide (wherein the TAT polypeptide may be expressed either by the cancer cell itself or a cell that produces polypeptide(s) that have a growth potentiating effect on cancer cells), wherein the method comprises contacting the TAT polypeptide with an antibody, an oligopeptide or a small organic molecule that binds to the TAT polypeptide, thereby antagonizing the growth-potentiating activity of the TAT polypeptide and, in turn, inhibiting the growth of the cancer cell. Preferably the growth of the cancer cell is completely inhibited. Even more preferably, binding of the antibody, oligopeptide or small organic molecule to the TAT polypeptide induces the death of the cancer cell. Optionally, the antibody is a monoclonal antibody, antibody fragment, chimeric antibody, humanized antibody, or single-chain antibody. Antibodies, TAT binding oligopeptides and TAT binding organic molecules employed in the methods of the present invention may optionally be conjugated to a growth inhibitory agent or cytotoxic agent such as a toxin, including, for example, a maytansinoid or calicheamicin, an antibiotic, a radioactive isotope, a nucleolytic enzyme, or the like. The antibodies and TAT binding oligopeptides employed in the methods of the present invention may optionally be produced in CHO cells or bacterial cells.

Yet another embodiment of the present invention is directed to a method of therapeutically treating a tumor in a mammal, wherein the growth of said tumor is at least in part dependent upon the growth potentiating effect(s) of a TAT polypeptide, wherein the method comprises administering to the mammal a therapeutically effective amount of an antibody, an oligopeptide or a small organic molecule that binds to the TAT polypeptide, thereby antagonizing the growth potentiating activity of said TAT polypeptide and resulting in the effective therapeutic treatment of the tumor. Optionally, the antibody is a monoclonal antibody, antibody fragment, chimeric antibody, humanized antibody, or single-chain antibody. Antibodies, TAT binding oligopeptides and TAT binding organic molecules employed in the methods of the present invention may optionally be conjugated to a growth inhibitory agent or cytotoxic agent such as a toxin, including, for example, a maytansinoid or calicheamicin, an antibiotic, a radioactive isotope, a nucleolytic enzyme, or the like. The antibodies and oligopeptides employed in the methods of the present invention may optionally be produced in CHO cells or bacterial cells.

Yet further embodiments of the present invention will be evident to the skilled artisan upon a reading of the present specification.

### BRIEF DESCRIPTION OF THE DRAWINGS

In the list of figures for the present application, specific cDNA sequences which are upregulated in certain tumor tissues as compared to their normal tissue counterparts are individually identified with a designation beginning with the letters “DNA” followed by a specific numerical designation. A full or partial length protein sequence that is encoded by a cDNA sequence identified and shown herein is individually identified with a designation beginning with the letters “PRO” followed by a specific numerical designation. Figures showing encoded amino acid sequences immediately follow the figure showing the cDNA sequence encoding that specific amino acid sequence. If start and/or stop codons have been identified in a cDNA sequence shown in the attached figures, they are shown in bold and underlined font.

### LIST OF FIGURES

FIG. 1: DNA323717,XM_{—}059201,gen.XM_{—}059201

FIG. 2: DNA323718,XM_{—}117159,gen.XM_{—}117159

FIG. 3: DNA323719,XM_{—}114062,gen.XM_{—}114062

FIG. 4: DNA323720,XM_{—}086178,gen.XM_{—}086178

FIG. 5: PRO80480

FIG. 6: DNA323721,XM_{—}051556,gen.XM_{—}051556

FIG. 7: PRO80481

FIG. 8: DNA323722,NM_{—}017891,gen.NM_{—}017891

FIG. 9: PRO80482

FIG. 10: DNA323723,NM_{—}018188,gen.NM_{—}018188

FIG. 11: PRO80483

FIG. 12: DNA323724,NM_{—}002617,gen.NM_{—}002617

FIG. 13: PRO23746

FIG. 14: DNA323725,XM_{—}049742,gen.XM_{—}049742

FIG. 15: DNA323726,NM_{—}033534,gen.NM_{—}033534

FIG. 16: PRO80484

FIG. 17: DNA323727,NM_{—}014188,gen.NM_{—}014188

FIG. 18: PRO80485

FIG. 19: DNA323728,XM_{—}086180,gen.XM_{—}086180

FIG. 20: DNA323729,XM_{—}166599,gen.XM_{—}166599

FIG. 21: PRO80487

FIG. 22: DNA323730,NM_{—}017900,gen.NM_{—}017900

FIG. 23: PRO80488

FIG. 24: DNA323731,XM_{—}001589,gen.XM_{—}001589

FIG. 25: PRO80489

FIG. 26: DNA323732,NM_{—}016176,gen.NM_{—}016176

FIG. 27: PRO80490

FIG. 28: DNA323733,XM_{—}117692,gen.XM_{—}117692

FIG. 29: DNA323734,XM_{—}086360,gen.XM_{—}086360

FIG. 30: PRO80492

FIG. 31: DNA287173,NM_{—}001428,gen.NM_{—}001428

FIG. 32: PRO69463

FIG. 33: DNA323735,XM_{—}001299,gen.XM_{—}001299

FIG. 34: DNA323736,NM_{—}000983,gen.NM_{—}000983

FIG. 35: PRO80493

FIG. 36A-B: DNA227821,NM_{—}014851,gen.NM_{—}014851

FIG. 37: PRO38284

FIG. 38A-B: DNA323737,XM_{—}086204,gen.XM_{—}086204

FIG. 39: PRO80494

FIG. 40: DNA323738,XM_{—}030920,gen.XM_{—}030920

FIG. 41: DNA323739,NM_{—}018948,gen.NM_{—}018948

FIG. 42: DNA273712,NM_{—}007262,gen.NM_{—}007262

FIG. 43: PRO61679

FIG. 44: DNA151148,NM_{—}004781,gen.NM_{—}004781

FIG. 45: PRO12618

FIG. 46: DNA323740,XM_{—}086151,gen.XM_{—}086151

FIG. 47: PRO80497

FIG. 48: DNA171408,NM_{—}004401,gen.NM_{—}004401

FIG. 49: PRO20136

FIG. 50: DNA323741,NM_{—}003132,gen.NM_{—}003132

FIG. 51: PRO80498

FIG. 52: DNA323742,XM_{—}086586,gen.XM_{—}086586

FIG. 53: PRO80499

FIG. 54: DNA323743,XM_{—}086587,gen.XM_{—}086587

FIG. 55: DNA323744,XM_{—}059230,gen.XM_{—}059230

FIG. 56: PRO80501

FIG. 57A-B: DNA323745,XM_{—}048780,gen.XM_{—}048780

FIG. 58: DNA323746,XM_{—}053183,gen.XM_{—}053183

FIG. 59: DNA323747,XM_{—}165442,gen.XM_{—}165442

FIG. 60: DNA323748,NM_{—}033440,gen.NM_{—}033440

FIG. 61: PRO2269

FIG. 62: DNA323749,NM_{—}024329,gen.NM_{—}024329

FIG. 63: PRO80505

FIG. 64: DNA323750,XM_{—}018205,gen.XM_{—}018205

FIG. 65: PRO80506

FIG. 66: DNA323751,XM_{—}011650,gen.XM_{—}011650

FIG. 67: DNA323752,XM_{—}017315,gen.XM_{—}017315

FIG. 68A-B: DNA323753,XM_{—}030470,genXM_{—}030470

FIG. 69: DNA323754,NM_{—}004930,gen.NM_{—}004930

FIG. 70: PRO80510

FIG. 71: DNA323755,NM_{—}003689,gen.NM_{—}003689

FIG. 72: PRO80511

FIG. 73: DNA323756,NM_{—}016183,gen.NM_{—}016183

FIG. 74: PRO80512

FIG. 75: DNA323757,XM_{—}015234,gen.XM_{—}015234

FIG. 76A-B: DNA323758,XM_{—}027916,gen.XM_{—}027916

FIG. 77: DNA323759,XM_{—}033683,gen.XM_{—}033683

FIG. 78: DNA323760,XM_{—}001826,gen.XM_{—}001826

FIG. 79: DNA323761,XM_{—}033654,gen.XM_{—}033654

FIG. 80: PRO80517

FIG. 81: DNA323762,NM_{—}001791,gen.NM_{—}001791

FIG. 82: PRO26194

FIG. 83: DNA323763,NM_{—}005826,gen.NM_{—}005826

FIG. 84: PRO60815

FIG. 85: DNA323764,XM_{—}086357,gen.XM_{—}086357

FIG. 86: PRO80518

FIG. 87: DNA323765,NM_{—}000975,gen.NM_{—}000975

FIG. 88: PRO80519

FIG. 89: DNA323766,NM_{—}007260,gen.NM_{—}007260

FIG. 90: PRO61250

FIG. 91: DNA323767,NM_{—}017761,gen.NM_{—}017761

FIG. 92: PRO80520

FIG. 93: DNA323768,NM_{—}006625,gen.NM_{—}006625

FIG. 94: PRO22196

FIG. 95: DNA323769,NM_{—}054016,gen.NM_{—}054016

FIG. 96: PRO80521

FIG. 97: DNA323770,XM_{—}086375,gen.XM_{—}086375

FIG. 98: DNA323771,XM_{—}006290,gen.XM_{—}006290

FIG. 99: DNA323772,NM_{—}015484,gen.NM_{—}015484

FIG. 100: PRO80524

FIG. 101A-B: DNA323773,XM_{—}001616,gen.XM_{—}001616

FIG. 102: DNA323774,XM_{—}058240,gen.XM_{—}058240

FIG. 103: DNA323775,XM_{—}059117,gen.XM_{—}059117

FIG. 104: PRO80527

FIG. 105: DNA226262,NM_{—}005563,gen.NM_{—}005563

FIG. 106: PRO36725

FIG. 107: DNA323776,NM_{—}022778,gen.NM_{—}1022778

FIG. 108: PRO80528

FIG. 109: DNA323777,XM_{—}017846,gen.XM_{—}017846

FIG. 110: DNA323778,NM_{—}005517,gen.NM_{—}005517

FIG. 111: PRO80530

FIG. 112A-C: DNA323779,XM_{—}046918,gen.XM_{—}046918

FIG. 113: DNA323780,XM_{—}002114,gen.XM_{—}002114

FIG. 114: DNA323781,XM_{—}059066,gen.XM_{—}059066

FIG. 115: PRO80533

FIG. 116: DNA323782,NM_{—}018066,gen.NM_{—}018066

FIG. 117: PRO80534

FIG. 118: DNA323783,NM_{—}006600,gen.NM_{—}006600

FIG. 119: PRO80535

FIG. 120: DNA323784,XM_{—}059067,gen.XM_{—}059067

FIG. 121: PRO80536

FIG. 122: DNA323785,NM_{—}032872,gen.NM_{—}032872

FIG. 123: PRO080537

FIG. 124: DNA196349,NM_{—}006990,gen.NM_{—}006990

FIG. 125: PRO24856

FIG. 126: DNA323788,XM_{—}001640,gen.XM_{—}001640

FIG. 127: DNA323789,NM_{—}002946,gen.NM_{—}002946

FIG. 128: PRO59099

FIG. 129: DNA323790,XM_{—}114044,gen.XM_{—}114044

FIG. 130: DNA323791,XM_{—}059088,gen.XM_{—}059088

FIG. 131: DNA323792,NM_{—}031459,gen.NM_{—}031459

FIG. 132: PRO80542

FIG. 133: DNA323793,XM_{—}010664,gen.XM_{—}010664

FIG. 134: DNA323794,XM_{—}001812,gen.XM_{—}001812

FIG. 135: DNA323795,XM_{—}001807,gen.XM_{—}001807

FIG. 136: DNA323796,XM_{—}086444,gen.XM_{—}086444

FIG. 137: DNA323797,NM_{—}024640,gen.NM_{—}024640

FIG. 138: PRO80547

FIG. 139A-B: DNA323798,XM_{—}049310,gen.XM_{—}049310

FIG. 140: DNA323799,XM_{—}113374,gen.XM_{—}113374

FIG. 141: DNA323800,XM_{—}002105,gen.XM_{—}002105

FIG. 142: DNA323801,NM_{—}014571,gen.NM_{—}014571

FIG. 143: PRO80550

FIG. 144: DNA323802,XM_{—}165438,gen.XM_{—}165438

FIG. 145: DNA323803,XM_{—}029844,gen.XM_{—}029844

FIG. 146: DNA188748,NM_{—}006559,gen.NM_{—}006559

FIG. 147: PRO22304

FIG. 148: DNA323804,NM_{—}003757,gen.NM_{—}003757

FIG. 151: PRO80554

FIG. 152: DNA323806,NM_{—}023009,gen.NM_{—}023009

FIG. 153: PRO80555

FIG. 154: DNA323807,XM_{—}030423,gen.XM_{—}030423

FIG. 155A-B: DNA323808,XM_{—}036299,gen.XM_{—}036299

FIG. 156: PRO80557

FIG. 157: DNA227213,NM_{—}003680,gen.NM_{—}003680

FIG. 158: PRO37676

FIG. 159: DNA323809,NM_{—}006112,gen.NM_{—}006112

FIG. 160: PRO80558

FIG. 161: DNA323810,XM_{—}018136,gen.XM_{—}018136

FIG. 162: PRO80559

FIG. 163: DNA323811,XM_{—}117184,gen.XM_{—}117184

FIG. 164: PRO80560

FIG. 165: DNA323812,NM_{—}017825,gen.NM_{—}017825

FIG. 166: PRO80561

FIG. 167: DNA189315,NM_{—}014408,gen.NM_{—}014408

FIG. 168: PRO22262

FIG. 169A-B: DNA323813,XM_{—}029031,gen.XM_{—}029031

FIG. 170: PRO80562

FIG. 171: DNA323814,XM_{—}059171,gen.XM_{—}059171

FIG. 172: PRO80563

FIG. 173: DNA83085,NM_{—}000760,gen.NM_{—}000760

FIG. 174: PRO2583

FIG. 175: DNA323815,XM_{—}165984,gen.XM_{—}165984

FIG. 176: DNA323816,XM_{—}029842,gen.XM_{—}029842

FIG. 177: PRO2851

FIG. 178: DNA323817,XM_{—}086384,gen.XM_{—}86384

FIG. 179: PRO80565

FIG. 180A-C: DNA274487,NM_{—}014747,gen.NM_{—}014747

FIG. 181: PRO62389

FIG. 182: DNA323818,XM_{—}010712,gen.XM_{—}010712

FIG. 183: DNA323819,NM_{—}024664,gen.NM_{—}024664

FIG. 184: PRO80567

FIG. 185: DNA323820,XM_{—}059214,gen.XM_{—}059214

FIG. 186: PRO80568

FIG. 187: DNA323821,XM_{—}046349,gen.XM_{—}046349

FIG. 188: DNA103253,NM_{—}006516,gen.NM_{—}006516

FIG. 189: PRO4583

FIG. 190: DNA323822,XM_{—}086543,gen.XM_{—}086543

FIG. 191: PRO80570

FIG. 192: DNA274745,NM_{—}006824,gen.NM_{—}006824

FIG. 193: PRO62518

FIG. 194: DNA273060,NM_{—}001255,gen.NM_{—}001255

FIG. 195: PRO61125

FIG. 196: DNA323823,NM_{—}030587,gen.NM_{—}030587

FIG. 197: PRO80571

FIG. 198: DNA323824,XM_{—}097649,gen.XM_{—}097649

FIG. 199: DNA256503,NM_{—}003780,gen.NM_{—}003780

FIG. 200: PRO51539

FIG. 201: DNA323825,XM_{—}046450,gen.XM_{—}046450

FIG. 202A-B: DNA272024,NM_{—}014663,gen.NM_{—}014663

FIG. 203: PRO60298

FIG. 204: DNA323826,XM_{—}046565,gen.XM_{—}046565

FIG. 205: PRO80574

FIG. 206: DNA323827,NM_{—}024602,gen.NM_{—}024602

FIG. 207: PRO80575

FIG. 208: DNA323828,XM_{—}046557,gen.XM_{—}046557

FIG. 209: PRO80576

FIG. 210: DNA323829,NM_{—}001012,gen.NM_{—}001012

FIG. 211: PRO10760

FIG. 212: DNA323830,XM_{—}046551,gen.XM_{—}046551

FIG. 213A-B: DNA323831,XM_{—}027983,gen.XM_{—}027983

FIG. 214: DNA323832,XM_{—}086324,gen.XM_{—}086324

FIG. 215: PRO80579

FIG. 216: DNA323833,XM_{—}032391,gen.XM_{—}032391

FIG. 217: PRO80580

FIG. 218: DNA103214,NM_{—}006066,gen.NM_{—}006066

FIG. 219: PRO4544

FIG. 220: DNA304686,NM_{—}002574,gen.NM_{—}002574

FIG. 221: PRO71112

FIG. 222: DNA323834,NM_{—}032756,gen.NM_{—}032756

FIG. 223: PRO80581

FIG. 224: DNA323835,XM_{—}059133,gen.XM_{—}059133

FIG. 225: PRO80582

FIG. 226: DNA323836,XM_{—}027313,gen.XM_{—}027313

FIG. 227: PRO80583

FIG. 228: DNA323837,XM_{—}054868,gen.XM_{—}054868

FIG. 229: DNA323838,NM_{—}001262,gen.NM_{—}001262

FIG. 230: PRO59546

FIG. 231: DNA323839,XM_{—}086391,gen.XM_{—}086391

FIG. 232: PRO80584

FIG. 233: DNA323840,XM_{—}114798,gen.XM_{—}114798

FIG. 234: PRO80585

FIG. 235: DNA272748,NM_{—}002979,gen.NM_{—}002979

FIG. 236: PRO60860

FIG. 237: DNA323841,XM_{—}038911,gen.XM_{—}038911

FIG. 238: PRO80586

FIG. 239: DNA323842,NM_{—}018070,gen.NM_{—}018070

FIG. 240: PRO80587

FIG. 241: DNA323843,NM_{—}024603,gen.NM_{—}024603

FIG. 242: PRO80588

FIG. 243: DNA323844,XM_{—}086389,gen.XM_{—}086389

FIG. 244: DNA323845,XM_{—}038852,gen.XM_{—}038852

FIG. 245: DNA323846,NM_{—}032864,gen.NM_{—}032864

FIG. 246: PRO80591

FIG. 247: DNA323847,NM_{—}024586,gen.NM_{—}024586

FIG. 248: PRO80592

FIG. 249A-B: DNA323848,XM_{—}097565,gen.XM_{—}097565

FIG. 250: DNA323849,XM_{—}001472,gen.XM_{—}001472

FIG. 251A-C: DNA323850,XM_{—}055481,gen.XM_{—}055481

FIG. 252: PRO80593

FIG. 253: DNA323851,XM_{—}010615,gen.XM_{—}010615

FIG. 254A-B: DNA323852,XM_{—}089138,gen.XM_{—}089138

FIG. 255: PRO80595

FIG. 256A-B: DNA323853,XM_{—}059180,gen.XM_{—}59180

FIG. 257: DNA323854,XM_{—}015717,gen.XM_{—}015717

FIG. 258: PRO80597

FIG. 259: DNA323855,XM_{—}114125,gen.XM_{—}114125

FIG. 260: DNA323856,NM_{—}015640,gen.NM_{—}015640

FIG. 261: PRO80599

FIG. 262: DNA323857,NM_{—}017768,gen.NM_{—}017768

FIG. 263: PRO80600

FIG. 264: DNA323858,XM_{—}165977,gen.XM_{—}165977

FIG. 265: DNA323859,XM_{—}086343,gen.XM_{—}086343

FIG. 266: PRO80602

FIG. 267: DNA269708,NM_{—}007034,gen.NM_{—}007034

FIG. 268: PRO58118

FIG. 269: DNA323860,NM_{—}001554,gen.NM_{—}001554

FIG. 270: PRO80603

FIG. 271: DNA226260,NM_{—}006769,gen.NM_{—}006769

FIG. 272: PRO36723

FIG. 273: DNA323861,NM_{—}004261,gen.NM_{—}004261

FIG. 274: PRO8060

FIG. 275: DNA323862,XM_{—}165983,gen.XM_{—}165983

FIG. 276: DNA323863,XM_{—}016164,gen.XM_{—}016164

FIG. 277: DNA323864,XM_{—}086164,gen.XM_{—}086164

FIG. 278: PRO80607

FIG. 279: DNA323865,XM_{—}086165,gen.XM_{—}086165

FIG. 280: DNA323866,XM_{—}086167,gen.XM_{—}086167

FIG. 281: DNA323867,XM_{—}086166,gen.XM_{—}086166

FIG. 282: DNA323868,XM_{—}086138,gen.XM_{—}086138

FIG. 283: PRO80611

FIG. 284: DNA323869,NM_{—}000969,gen.NM_{—}000969

FIG. 285: PRO80612

FIG. 286: DNA323870,XM_{—}088863,gen.XM_{—}088863

FIG. 287: PRO80613

FIG. 288: DNA271003,NM_{—}003729,gen.NM_{—}003729

FIG. 289: PRO59332

FIG. 290: DNA323871,XM_{—}165981,gen.XM_{—}165981

FIG. 291: PRO80614

FIG. 292: DNA275139,NM_{—}013296,gen.NM_{—}013296

FIG. 293: PRO62849

FIG. 294: DNA323872,XM_{—}058702,gen.XM_{—}058702

FIG. 295: DNA323873,XM_{—}054978,gen.XM_{—}054978

FIG. 296: DNA323874,NM_{—}032636,gen.NM_{—}032636

FIG. 297: PRO80617

FIG. 298: DNA323875,NM_{—}006513,gen.NM_{—}006513

FIG. 299: PRO80618

FIG. 300: DNA323876,NM_{—}006621,gen.NM_{—}006621

FIG. 301: PRO80619

FIG. 302A-B: DNA323877,NM_{—}007158,gen.NM_{—}007158

FIG. 303: PRO80620

FIG. 304: DNA323878,XM_{—}086132,gen.XM_{—}086132

FIG. 305: PRO80621

FIG. 306: DNA323879,NM_{—}004000,gen.NM_{—}004000

FIG. 307: PRO80622

FIG. 308: DNA323880,NM_{—}001688,gen.NM_{—}001688

FIG. 309: PRO80623

FIG. 310: DNA323881,NM_{—}019099,gen.NM_{—}019099

FIG. 311: PRO80624

FIG. 312A-B: DNA323882,NM_{—}000701,gen.NM_{—}000701

FIG. 313: PRO80625

FIG. 314A-B: DNA323883,XM_{—}018332,gen.XM_{—}018332

FIG. 315A-B: DNA323884,XM_{—}040709,gen.XM_{—}040709

FIG. 316: PRO80627

FIG. 317: DNA323885,XM_{—}086518,gen.XM_{—}086518

FIG. 318A-D: DNA323886,XM_{—}034671,gen.XM_{—}034671

FIG. 319: DNA323887,XM_{—}034662,gen.XM_{—}034662

FIG. 320: PRO80630

FIG. 321: DNA323888,XM_{—}039721,gen.XM_{—}039721

FIG. 322: PRO80631

FIG. 323A-B: DNA323889,XM_{—}086397,gen.XM_{—}086397

FIG. 324A-B: DNA323890,XM_{—}086515,gen.XM_{—}086515

FIG. 325: PRO80633

FIG. 326: DNA323891,XM_{—}016480,gen.XM_{—}016480

FIG. 327: DNA323892,XM_{—}165975,gen.XM_{—}165975

FIG. 328: DNA323893,NM_{—}016361,gen.NM_{—}016361

FIG. 329: PRO231

FIG. 330: DNA323894,XM_{—}059210,gen.XM_{—}059210

FIG. 331: DNA323895,XM_{—}086296,gen.XM_{—}086296

FIG. 332: DNA323896,NM_{—}030920,gen.NM_{—}030920

FIG. 333: PRO80638

FIG. 334: DNA323897,NM_{—}016022,gen.NM_{—}016022

FIG. 335: PRO80639

FIG. 336: DNA323898,NM_{—}031901,gen.NM_{—}031901

FIG. 337: PRO80640

FIG. 338A-B: DNA323899,XM_{—}088788,gen.XM_{—}088788

FIG. 339: PRO80641

FIG. 340: DNA274759,NM_{—}005620,gen.NM_{—}005620

FIG. 341: PRO62529

FIG. 342: DNA323900,XM_{—}001468,gen.XM_{—}001468

FIG. 343: PRO49642

FIG. 344: DNA323901,NM_{—}006862,gen.NM_{—}006862

FIG. 345: PRO80642

FIG. 346: DNA227529,NM_{—}002796,gen.NM_{—}002796

FIG. 347: PRO37992

FIG. 348: DNA323902,NM_{—}002810,gen.NM_{—}002810

FIG. 349: PRO61638

FIG. 350: DNA290284,NM_{—}005997,gen.NM_{—}005997

FIG. 351: PRO70433

FIG. 352: DNA323903,XM_{—}097639,gen.XM_{—}097639

FIG. 353: DNA323904,XM_{—}041879,gen.XM_{—}041879

FIG. 354: DNA323905,XM_{—}041884,gen.XM_{—}041884

FIG. 355: PRO80644

FIG. 356: DNA225809,NM_{—}000396,gen.NM_{—}000396

FIG. 357: PRO36272

FIG. 358: DNA323906,NM_{—}025150,gen.NM_{—}025150

FIG. 359: PRO80645

FIG. 360: DNA323907,XM_{—}114098,gen.XM_{—}114098

FIG. 361: DNA323908,XM_{—}113369,gen.XM_{—}113369

FIG. 362: PRO80646

FIG. 363: DNA323909,XM_{—}099467,gen.XM_{—}099467

FIG. 364: DNA323910,NM_{—}002965,gen.NM_{—}002965

FIG. 365: PRO80648

FIG. 366: DNA323911,XM_{—}086400,gen.XM_{—}086400

FIG. 367: DNA210134,NM_{—}014624,gen.NM_{—}014624

FIG. 368: PRO33679

FIG. 369: DNA304666,NM_{—}002961,gen.NM_{—}002961

FIG. 370: PRO71093

FIG. 371: DNA304720,NM_{—}019554,gen.NM_{—}019554

FIG. 372: PRO71146

FIG. 373: DNA323912,XM_{—}165976,gen.XM_{—}165976

FIG. 374: DNA227577,NM_{—}006271,gen.NM_{—}006271

FIG. 375: PRO38040

FIG. 376: DNA323913,XM_{—}114097,gen.XM_{—}114097

FIG. 377: DNA323914,XM_{—}040009,gen.XM_{—}040009

FIG. 378: PRO80651

FIG. 379: DNA323915,NM_{—}024330,gen.NM_{—}024330

FIG. 380: PRO703

FIG. 381: DNA323916,NM_{—}012437,gen.NM_{—}012437

FIG. 382: PRO80652

FIG. 383: DNA323917,XM_{—}086271,gen.XM_{—}086271

FIG. 384: DNA323918,XM_{—}114055,gen.XM_{—}114055

FIG. 385: PRO37535

FIG. 386: DNA323919,XM_{—}113360,gen.XM_{—}113360

FIG. 387: PRO80654

FIG. 388: DNA323920,XM_{—}086564,gen.XM_{—}086564

FIG. 389: DNA323921,NM_{—}005973,gen.NM_{—}005973

FIG. 390: PRO80656

FIG. 391: DNA323922,XM_{—}044077,gen.XM_{—}044077

FIG. 392: DNA323923,NM_{—}001878,gen.NM_{—}001878

FIG. 393: PRO80657

FIG. 394: DNA323924,NM_{—}021948,gen.NM_{—}021948

FIG. 395: PRO6018

FIG. 396: DNA273088,NM_{—}006365,gen.NM_{—}006365

FIG. 397: PRO61146

FIG. 398: DNA323925,XM_{—}044127,gen.XM_{—}044127

FIG. 399: PRO80658

FIG. 400: DNA323926,XM_{—}053245,gen.XM_{—}053245

FIG. 401: PRO80659

FIG. 402: DNA257916,NM_{—}032323,gen.NM_{—}032323

FIG. 403: PRO52449

FIG. 404: DNA323927,NM_{—}005572,gen.NM_{—}005572

FIG. 405: PRO80660

FIG. 406: DNA323928,XM_{—}044166,gen.XM_{—}044166

FIG. 407: PRO80661

FIG. 408: DNA323929,XM_{—}044128,gen.XM_{—}044128

FIG. 409: DNA226125,NM_{—}003145,gen.NM_{—}003145

FIG. 410: PRO36588

FIG. 411A-B: DNA323930,XM_{—}044172,gen.XM_{—}044172

FIG. 412: DNA323931,NM_{—}032292,gen.NM_{—}032292

FIG. 413: PRO80664

FIG. 414: DNA323932,NM_{—}004632,gen.NM_{—}004632

FIG. 415: PRO80665

FIG. 416: DNA323933,XM_{—}044075,gen.XM_{—}044075

FIG. 417: PRO80666

FIG. 418: DNA323934,NM_{—}018253,gen.NM_{—}018253

FIG. 419: PRO80667

FIG. 420: DNA323935,NM_{—}018116,gen.NM_{—}018116

FIG. 421: PRO80668

FIG. 422: DNA323936,NM_{—}002004,gen.NM_{—}002004

FIG. 423: PRO80669

FIG. 424: DNA323937,NM_{—}005698,gen.NM_{—}005698

FIG. 425: PRO80670

FIG. 426: DNA323938,NM_{—}052837,gen.NM_{—}052837

FIG. 427: PRO80671

FIG. 428: DNA194600,NM_{—}006589,gen.NM_{—}006589

FIG. 429: PRO23942

FIG. 430: DNA323939,XM_{—}086567,gen.XM_{—}086567

FIG. 431: PRO80672

FIG. 432: DNA323940,XM_{—}086552,gen.XM_{—}086552

FIG. 433: DNA323941,XM_{—}036744,gen.XM_{—}036744

FIG. 434: DNA323942,NM_{—}130898,gen.NM_{—}130898

FIG. 435: PRO80675

FIG. 436: DNA226793,NM_{—}006694,gen.NM_{—}006694

FIG. 437: PRO37256

FIG. 438: DNA294794,NM_{—}002870,gen.NM_{—}002870

FIG. 439: PRO70754

FIG. 440: DNA323943,NM_{—}001030,gen.NM_{—}001030

FIG. 441: PRO80676

FIG. 442: DNA323944,XM_{—}036829,gen.XM_{—}036829

FIG. 443: PRO80677

FIG. 444: DNA323945,NM_{—}015449,gen.NM_{—}015449

FIG. 445: PRO80678

FIG. 446: DNA323946,NM_{—}014847,gen.NM_{—}014847

FIG. 447: PRO80679

FIG. 448: DNA323947,XM_{—}036934,gen.XM_{—}036934

FIG. 449: PRO80680

FIG. 450A-B: DNA323948,XM_{—}036845,gen.XM_{—}036845

FIG. 451: DNA323949,XM_{—}010636,gen.XM_{—}010636

FIG. 452: DNA323950,NM_{—}006556,gen.NM_{—}006556

FIG. 453: PRO62574

FIG. 454: DNA323951,XM_{—}034082,gen.XM_{—}034082

FIG. 455: DNA323952,NM_{—}025207,gen.NM_{—}025207

FIG. 456: PRO80684

FIG. 457: DNA103436,NM_{—}003815,gen.NM_{—}003815

FIG. 458: PRO4763

FIG. 459: DNA323953,NM_{—}003516,gen.NM_{—}003516

FIG. 460: PRO80685

FIG. 461: DNA323954,NM_{—}005850,gen.NM_{—}005850

FIG. 462: PRO59725

FIG. 463A-B: DNA323955,NM_{—}014849,gen.NM_{—}014849

FIG. 464: PRO80686

FIG. 465: DNA323956,XM_{—}059094,gen.XM_{—}059094

FIG. 466: DNA323957,XM_{—}058247,gen.XM_{—}058247

FIG. 467: PRO80688

FIG. 468: DNA323958,NM_{—}003779,gen.NM_{—}003779

FIG. 469: PRO80689

FIG. 470: DNA323959,NM_{—}004550,gen.NM_{—}004550

FIG. 471: PRO58974

FIG. 472: DNA323960,XM_{—}085581,gen.XM_{—}085581

FIG. 473: DNA323961,XM_{—}113379,gen.XM_{—}113379

FIG. 474: DNA226619,NM_{—}003564,gen.NM_{—}003564

FIG. 475: PRO37082

FIG. 476A-B: DNA323962,XM_{—}049680,gen.XM_{—}049680

FIG. 477: DNA323963,XM_{—}165443,gen.XM_{—}165443

FIG. 478: PRO80693

FIG. 479: DNA323964,XM_{—}086381,gen.XM_{—}086381

FIG. 480: PRO80694

FIG. 481A-B: DNA323965,NM_{—}002857,gen.NM_{—}002857

FIG. 482: PRO80695

FIG. 483A-B: DNA323966,XM_{—}049690,gen.XM_{—}049690

FIG. 484: DNA323967,XM_{—}114153,gen.XM_{—}114153

FIG. 485: DNA323968,XM_{—}086378,gen.XM_{—}086378

FIG. 486: DNA323969,XM_{—}001897,gen.XM_{—}001897

FIG. 487: PRO10002

FIG. 488: DNA323970,NM_{—}052862,gen.NM_{—}052862

FIG. 489: PRO80699

FIG. 490: DNA323971,XM_{—}086481,gen.XM_{—}086481

FIG. 491: PRO8070

FIG. 492: DNA323972,XM_{—}059191,gen.XM_{—}059191

FIG. 493: DNA323973,XM_{—}086485,gen.XM_{—}086485

FIG. 494: DNA323974,XM_{—}086484,gen.XM_{—}086484

FIG. 495: DNA323975,XM_{—}047479,gen.XM_{—}047479

FIG. 496: PRO80704

FIG. 497: DNA323976,NM_{≠}003617,gen.NM_{—}003617

FIG. 498: PRO37806

FIG. 499: DNA254298,NM_{—}025226,gen.NM_{—}025226

FIG. 500: PRO49409

FIG. 501: DNA323977,XM_{—}034000,gen.XM_{—}034000

FIG. 502: PRO80705

FIG. 503: DNA323978,NM_{—}032738,gen.NM_{—}032738

FIG. 504: PRO329

FIG. 505: DNA323979,NM_{—}000569,gen.NM_{—}000569

FIG. 506: PRO80706

FIG. 507: DNA323980,XM_{—}088945,gen.XM_{—}088945

FIG. 508: PRO80707

FIG. 509: DNA323981,XM_{—}060331,gen.XM_{—}060331

FIG. 510: PRO80708

FIG. 511: DNA323982,NM_{—}004905,gen.NM_{—}004905

FIG. 512: PRO80709

FIG. 513: DNA323983,NM_{—}017847,gen.NM_{—}017847

FIG. 514: PRO80710

FIG. 515A-B: DNA323984,XM_{—}051877,gen.XM_{—}051877

FIG. 516: PRO62077

FIG. 517: DNA323985,NM_{—}005717,gen.NM_{—}005717

FIG. 518: PRO80711

FIG. 519A-B: DNA271986,NM_{—}014837,gen.NM_{—}014837

FIG. 520: PRO60261

FIG. 521A-B: DNA323986,XM_{—}056923,gen.XM_{—}056923

FIG. 522: DNA323987,XM_{—}046464,gen.XM_{—}046464

FIG. 523: DNA323988,XM_{—}002068,gen.XM_{—}002068

FIG. 524A-B: DNA323989,XM_{—}001289,gen.XM_{—}001289

FIG. 525: DNA323990,XM_{—}114109,gen.XM_{—}114109

FIG. 526: PRO80714

FIG. 527: DNA323991,NM_{—}022371,gen.NM_{—}022371

FIG. 528: PRO80715

FIG. 529: DNA323992,NM_{—}004673,gen.NM_{—}004673

FIG. 530: PRO188

FIG. 531: DNA323993,XM_{—}060517,gen.XM_{—}060517

FIG. 532: DNA323994,XM_{—}165978,gen.XM_{—}165978

FIG. 533: PRO80717

FIG. 534: DNA323995,XM_{—}117181,gen.XM_{—}117181

FIG. 535: DNA323996,NM_{—}018122,gen.NM_{—}018122

FIG. 536: PRO80719

FIG. 537: DNA323997,XM_{—}042967,gen.XM_{—}042967

FIG. 538: DNA323998,XM_{—}086494,gen.XM_{—}086494

FIG. 539: PRO80720

FIG. 540: DNA290234,NM_{—}002923,gen.NM_{—}002923

FIG. 541: PRO70333

FIG. 542: DNA323999,XM_{—}086328,gen.XM_{—}086328

FIG. 543: DNA324000,XM_{—}086282,gen.XM_{—}086282

FIG. 544: DNA324001,XM_{—}053633,gen.XM_{—}053633

FIG. 545: DNA256905,NM_{—}138391,gen.NM_{—}138391

FIG. 546: PRO51836

FIG. 547: DNA324002,XM_{—}015434,gen.XM_{—}015434

FIG. 548: DNA324003,NM_{—}006763,gen.NM_{—}006763

FIG. 549: PRO80725

FIG. 550: DNA227246,NM_{—}005686,gen.NM_{—}005686

FIG. 551: PRO37709

FIG. 552: DNA324004,XM_{—}058405,gen.XM_{—}058405

FIG. 553A-B: DNA226005,NM_{—}000228,gen.NM_{—}000228

FIG. 554: PRO36468

FIG. 555: DNA324005,NM_{—}015714,gen.NM_{—}015714

FIG. 556: PRO11582

FIG. 557: DNA324006,XM_{—}086142,gen.XM_{—}086142

FIG. 558: DNA83046,NM_{—}000574,gen.NM_{—}000574

FIG. 559: PRO2569

FIG. 560A-B: DNA324007,XM_{—}114030,gen.XM_{—}114030

FIG. 561: DNA324008,XM_{—}097519,gen.XM_{—}097519

FIG. 562: DNA324009,XM_{—}059120,gen.XM_{—}059120

FIG. 563: PRO80730

FIG. 564: DNA324010,NM_{—}016456,gen.NM_{—}016456

FIG. 565: PRO1248

FIG. 566: DNA324011,XM_{—}036556,gen.XM_{—}036556

FIG. 567: DNA324012,XM_{—}001914,gen.XM_{—}001914

FIG. 568: DNA324013,XM_{—}001916,gen.XM_{—}001916

FIG. 569: DNA324014,NM_{—}018085,gen.NM_{—}018085

FIG. 570: PRO80734

FIG. 571: DNA324015,NM_{—}006335,gen.NM_{—}006335

FIG. 572: PRO80735

FIG. 573: DNA324016,XM_{—}036500,gen.XM_{—}036500

FIG. 574: PRO80736

FIG. 575: DNA324017,XM_{—}036507,gen.XM_{—}036507

FIG. 576: DNA196344,NM_{—}004767,gen.NM_{—}004767

FIG. 577: PRO24851

FIG. 578: DNA247474,NM_{—}014176,gen.NM_{—}014176

FIG. 579: PRO44999

FIG. 580A-B: DNA324018,XM_{—}084055,gen.XM_{—}084055

FIG. 581: DNA324019,XM_{—}010682,gen.XM_{—}010682

FIG. 582: DNA324020,XM_{—}117185,gen.XM_{—}117185

FIG. 583: DNA324021,XM_{—}055880,gen.XM_{—}055880

FIG. 584: PRO80740

FIG. 585: DNA193882,NM_{—}014184,gen.NM_{—}014184

FIG. 586: PRO23300

FIG. 587: DNA324022,NM_{—}018212,gen.NM_{—}018212

FIG. 588: PRO80741

FIG. 589: DNA324023,XM_{—}086431,gen.XM_{—}086431

FIG. 590: PRO80742

FIG. 591: DNA324024,XM_{—}037329,gen.XM_{—}037329

FIG. 592: DNA324025,XM_{—}086432,gen.XM_{—}086432

FIG. 593A-B: DNA324026,XM_{—}010732,gen.XM_{—}010732

FIG. 594: DNA227504,NM_{—}000447,gen.NM_{—}000447

FIG. 595: PR037967

FIG. 596: DNA324027,NM_{—}012486,gen.NM_{—}012486

FIG. 597: PRO80745

FIG. 598A-B: DNA324028,XM_{—}113361,gen.XM_{—}113361

FIG. 599A-B: DNA324029,XM_{—}001958,gen.XM_{—}001958

FIG. 600: DNA324030,XM_{—}016199,gen.XM_{—}016199

FIG. 601: DNA324031,XM_{—}086244,gen.XM_{—}086244

FIG. 602: DNA324032,XM_{—}086245,gen.XM_{—}086245

FIG. 603: DNA254346,NM_{—}024709,gen.NM_{—}024709

FIG. 604: PRO49457

FIG. 605: DNA324033,XM_{—}088107,gen.XM_{—}088107

FIG. 606: DNA324034,NM_{—}032890,gen.NM_{—}032890

FIG. 607: PRO80752

FIG. 608: DNA324035,XM_{—}052974,gen.XM_{—}052974

FIG. 609: PRO80753

FIG. 610: DNA324036,XM_{—}047499,gen.XM_{—}047499

FIG. 611: PRO80754

FIG. 612: DNA324037,NM_{—}000858,gen.NM_{—}000858

FIG. 613: PRO80755

FIG. 614: DNA324038,NM_{—}024319,gen.NM_{—}024319

FIG. 615: PRO80756

FIG. 616: DNA324039,XM_{—}047545,gen.XM_{—}047545

FIG. 617: PRO4914

FIG. 618A-B: DNA324040,XM_{—}056884,gen.XM_{—}056884

FIG. 619: DNA324041,XM_{—}098599,gen.XM_{—}098599

FIG. 620: DNA324042,XM_{—}165439,gen.XM_{—}165439

FIG. 621: PRO80759

FIG. 622: DNA324043,XM_{—}089030,gen.XM_{—}089030

FIG. 623: PRO80760

FIG. 624: DNA82328,NM_{—}000029,gen.NM_{—}000029

FIG. 625: PRO1707

FIG. 626: DNA324044,NM_{—}014236,gen.NM_{—}014236

FIG. 627: PRO80761

FIG. 628: DNA324045,XM_{—}056970,gen.XM_{—}056970

FIG. 629: PRO80762

FIG. 630: DNA324046,NM_{—}032324,gen.NM_{—}032324

FIG. 631: PRO80763

FIG. 632: DNA324047,XM_{—}086257,gen.XM_{—}086257

FIG. 633: PRO80764

FIG. 634: DNA324048,XM_{—}114137,gen.XM_{—}114137

FIG. 635: PRO80765

FIG. 636: DNA324049,NM_{—}000143,gen.NM_{—}000143

FIG. 637: PRO62607

FIG. 638: DNA324050,XM_{—}090833,gen.XM_{—}090833

FIG. 639: DNA324051,NM_{—}130398,gen.NM_{—}130398

FIG. 640: PRO80767

FIG. 641: DNA324052,XM_{—}117196,gen.XM_{—}117196

FIG. 642: DNA324053,XM_{—}018041,gen.XM_{—}018041

FIG. 643: DNA324054,NM_{—}001011,gen.NM_{—}001011

FIG. 644: PRO10692

FIG. 645: DNA324055,NM_{—}024027,gen.NM_{—}024027

FIG. 646: PRO1182

FIG. 647: DNA324056,NM_{—}016030,gen.NM_{—}016030

FIG. 648: PRO80770

FIG. 649: DNA103217,NM_{—}003310,gen.NM_{—}003310

FIG. 650: PRO4547

FIG. 651: DNA275195,NM_{—}001034,gen.NM_{—}001034

FIG. 652: PRO62893

FIG. 653: DNA324057,XM_{—}059368,gen.XM_{—}059368

FIG. 654: PRO80771

FIG. 655: DNA324058,NM_{—}006826,gen.NM_{—}006826

FIG. 656: PRO70258

FIG. 657: DNA324059,NM_{—}005378,gen.NM_{—}005378

FIG. 658: PRO80772

FIG. 659: DNA324060,NM_{—}002539,gen.NM_{—}002539

FIG. 660: PRO80773

FIG. 661: DNA324061,XM_{—}096149,gen.XM_{—}096149

FIG. 662: DNA275049,NM_{—}004939,gen.NM_{—}004939

FIG. 663: PRO62770

FIG. 664A-B: DNA324062,XM_{—}036450,gen.XM_{—}036450

FIG. 665: DNA324063,XM_{—}103946,gen.XM_{—}103946

FIG. 666: PRO80775

FIG. 667: DNA324064,NM_{—}014713,gen.NM_{—}014713

FIG. 668: PRO80776

FIG. 669: DNA324065,XM_{—}087206,gen.XM_{—}087206

FIG. 670: DNA324066,NM_{—}106552,gen.NM_{—}106552

FIG. 671: PRO80778

FIG. 672: DNA324067,XM_{—}092135,gen.XM_{—}092135

FIG. 673: PRO80779

FIG. 674: DNA324068,NM_{—}017910,gen.NM_{—}017910

FIG. 675: PRO80780

FIG. 676: DNA324069,XM_{—}092517,gen.XM_{—}092517

FIG. 677: PRO80781

FIG. 678A-B: DNA324070,NM_{—}025203,gen.NM_{—}025203

FIG. 679: PRO80782

FIG. 680: DNA324071,XM_{—}002480,gen.XM_{—}002480

FIG. 681: DNA324072,NM_{—}002707,gen.NM_{—}002707

FIG. 682: PRO12199

FIG. 683: DNA324073,XM_{—}087151,gen.XM_{—}087151

FIG. 684: DNA227165,NM_{—}014748,gen.NM_{—}014748

FIG. 685: PRO37628

FIG. 686: DNA324074,NM_{—}015636,gen.NM_{—}015636

FIG. 687: PRO80785

FIG. 688: DNA273800,NM_{—}001521,gen.NM_{—}001521

FIG. 689: PRO61761

FIG. 690: DNA324075,XM_{—}047175,gen.XM_{—}047175

FIG. 691: PRO80786

FIG. 692A-B: DNA324076,NM_{—}004341,gen.NM_{—}004341

FIG. 693: PRO80787

FIG. 694: DNA324077,NM_{—}016085,gen.NM_{—}016085

FIG. 695: PRO80788

FIG. 696: DNA324078,NM_{—}080592,gen.NM_{—}080592

FIG. 697: PRO80789

FIG. 698: DNA227545,NM_{—}021095,gen.NM_{—}021095

FIG. 699: PRO38008

FIG. 700: DNA324079,XM_{—}002435,gen.XM_{—}002435

FIG. 701: DNA324080,NM_{—}000221,gen.NM_{—}000221

FIG. 702: PRO80790

FIG. 703: DNA271243,NM_{—}006488,gen.NM_{—}006488

FIG. 704: PRO59558

FIG. 705: DNA324081,NM_{—}007046,gen.NM_{—}007046

FIG. 706: PRO9886

FIG. 707: DNA324082,NM_{—}021831,gen.NM_{—}021831

FIG. 708: PRO80791

FIG. 709: DNA324083,NM_{—}020134,gen.NM_{—}020134

FIG. 710: PRO80792

FIG. 711: DNA103593,NM_{—}000183,gen.NM_{—}000183

FIG. 712: PRO4917

FIG. 713: DNA324084,NM_{—}000182,gen.NM_{—}000182

FIG. 714: PRO80793

FIG. 715: DNA324085,XM_{—}097976,gen.XM_{—}097976

FIG. 716A-B: DNA324086,XM_{—}039712,gen.XM_{—}039712

FIG. 717: DNA324087,NM_{—}022552,gen.NM_{—}022552

FIG. 718: PRO80796

FIG. 719: DNA324088,NM_{—}024572,gen.NM_{—}024572

FIG. 720: PRO80797

FIG. 721: DNA324089,NM_{—}018607,gen.NM_{—}018607

FIG. 722: PRO80798

FIG. 723: DNA324090,XM_{—}165448,gen.XM_{—}165448

FIG. 724: PRO80799

FIG. 725: DNA324091,XM_{—}087195,gen.XM_{—}087195

FIG. 726: DNA324092,XM_{—}087193,gen.XM_{—}087193

FIG. 727: DNA324093,NM_{—}138801,gen.NM_{—}138801

FIG. 728: PRO80802

FIG. 729: DNA324094,XM_{—}098004,gen.XM_{—}098004

FIG. 730: PRO80803

FIG. 731: DNA324095,XM_{—}031519,gen.XM_{—}031519

FIG. 732: PRO80804

FIG. 733A-B: DNA324096,XM_{—}031527,gen.XM_{—}031527

FIG. 734: DNA324097,XM_{—}038576,gen.XM_{—}038576

FIG. 735: PRO80806

FIG. 736: DNA324098,XM_{—}117264,gen.XM_{—}117264

FIG. 737: PRO80807

FIG. 738A-B: DNA324099,XM_{—}031626,gen.XM_{—}031626

FIG. 739: PRO80808

FIG. 740: DNA324100,XM_{—}057664,gen.XM_{—}057664

FIG. 741: DNA226428,NM_{—}000251,gen.NM_{—}000251

FIG. 742: PRO36891

FIG. 743: DNA324101,XM_{—}087211,gen.XM_{—}087211

FIG. 744A-B: DNA275066,NM_{—}000179,gen.NM_{—}000179

FIG. 745: PRO62786

FIG. 746A-C: DNA270154,NM_{—}003128,gen.NM_{—}003128

FIG. 747: PRO58543

FIG. 748: DNA324102,XM_{—}087051,gen.XM_{—}087051

FIG. 749: DNA324103,NM_{—}002954,gen.NM_{—}002954

FIG. 750: PRO62239

FIG. 751: DNA271060,NM_{—}002453,gen.NM_{—}002453

FIG. 752: PRO59384

FIG. 753: DNA324104,XM_{—}048088,gen.XM_{—}048088

FIG. 754: PRO80811

FIG. 755: DNA324105,XM_{—}010886,gen.XM_{—}010886

FIG. 756: PRO80812

FIG. 757: DNA324106,XM_{—}045283,gen.XM_{—}045283

FIG. 758: PRO80813

FIG. 759: DNA324107,NM_{—}006430,gen.NM_{—}006430

FIG. 760: PRO80814

FIG. 761A-B: DNA324108,NM_{—}003400,gen.NM_{—}003400

FIG. 762: PRO59544

FIG. 763: DNA324109,XM_{—}018301,gen.XM_{—}018301

FIG. 764: DNA324110,NM_{—}005917,gen.NM_{—}005917

FIG. 765: PRO4918

FIG. 766: DNA324111,XM_{—}016843,gen.XM_{—}016843

FIG. 767: PRO80816

FIG. 768: DNA324112,XM_{—}088638,gen.XM_{—}088638

FIG. 769: PRO80817

FIG. 770: DNA324113,XM_{—}002647,gen.XM_{—}002647

FIG. 771: DNA324114,XM_{—}010881,gen.XM_{—}010881

FIG. 772: DNA324115,XM_{—}087069,gen.XM_{—}087069

FIG. 773: DNA324116,XM_{—}016625,gen.Xm_{—}087069

FIG. 774: PRO80820

FIG. 775: DNA324117,XM_{—}087068,gen.XM_{—}087068

FIG. 776: DNA324118,XM_{—}002674,gen.XM_{—}002674

FIG. 777: DNA324119,XM_{—}065884,gen.XM_{—}065884

FIG. 778: PRO80823

FIG. 779A-B: DNA324120,XM_{—}002739,gen.XM_{—}002739

FIG. 780: DNA324121,XM_{—}031596,gen.XM_{—}031596

FIG. 781: PRO61325

FIG. 782: DNA324122,XM_{—}031585,gen.XM_{—}031585

FIG. 783: DNA324123,XM_{—}031586,gen.XM_{—}031586

FIG. 784: DNA324124,XM_{—}018039,gen.XM_{—}018039

FIG. 785: DNA324125,NM_{—}032822,gen.NM_{—}032822

FIG. 786: PRO80827

FIG. 787A-B: DNA324126,XM_{—}096172,gen.XM_{—}096172

FIG. 788A-B: DNA324127,XM_{—}002727,gen.XM_{—}002727

FIG. 789: DNA324128,NM_{—}003124,gen.NM_{—}003124

FIG. 790: PRO80830

FIG. 791: DNA324129,XM_{—}086980,gen.XM_{—}086980

FIG. 792: DNA227795,NM_{—}006429,gen.NM_{—}006429

FIG. 793: PRO38258

FIG. 794: DNA287167,NM_{—}006636,gen.NM_{—}006636

FIG. 795: PRO59136

FIG. 796: DNA324130,NM_{—}033046,gen.NM_{—}033046

FIG. 797: PRO80832

FIG. 798: DNA324131,NM_{—}133637,gen.NM_{—}133637

FIG. 799: PRO80833

FIG. 800: DNA324132,XM_{—}035220,gen.XM_{—}035220

FIG. 801: DNA324133,NM_{—}013247,gen.NM_{—}013247

FIG. 802: PRO80835

FIG. 803: DNA227528,NM_{—}021103,gen.NM_{—}021103

FIG. 804: PRO37991

FIG. 805: DNA324134,XM_{—}086920,gen.XM_{—}086920

FIG. 806: DNA150725,NM_{—}001747,gen.NM_{—}001747

FIG. 807: PRO12792

FIG. 808: DNA324135,NM_{—}005911,gen.NM_{—}005911

FIG. 809: PRO80837

FIG. 810: DNA324136,NM_{—}032827,gen.NM_{—}032827

FIG. 811: PRO80838

FIG. 812: DNA324137,NM_{—}017952,gen.NM_{—}017952

FIG. 813: PRO80839

FIG. 814: DNA227190,NM_{—}006839,gen.NM_{—}006839

FIG. 815: PRO37653

FIG. 816: DNA324138,XM_{—}114215,gen.XM_{—}114215

FIG. 817: DNA324139,XM_{—}052989,gen.XM_{—}052989

FIG. 818: DNA324140,XM_{—}049116,gen.XM_{—}049116

FIG. 819: PRO80842

FIG. 820A-B: DNA324141,XM_{—}049108,gen.XM_{—}049108

FIG. 821: PRO80843

FIG. 822: DNA324142,XM_{—}049113,gen.XM_{—}049113

FIG. 823: DNA324143,XM_{—}002611,gen.XM_{—}002611

FIG. 824A-B: DNA324144,XM_{—}114247,gen.XM_{—}114247

FIG. 825: DNA324145,NM_{—}017789,gen.NM_{—}017789

FIG. 826: PRO80846

FIG. 827: DNA324146,NM_{—}001862,gen.NM_{—}001862

FIG. 828: PRO80847

FIG. 829: DNA324147,NM_{—}005783,gen.NM_{—}005783

FIG. 830: PRO80848

FIG. 831A-B: DNA324148,XM_{—}037108,gen.XM_{—}037108

FIG. 832: DNA324149,NM_{—}000993,gen.NM_{—}000993

FIG. 833: PRO11197

FIG. 834: DNA324150,NM_{—}017546,gen.NM_{—}017546

FIG. 835: PRO80850

FIG. 836: DNA324151,NM_{—}001450,gen.NM_{—}001450

FIG. 837: PRO80851

FIG. 838: DNA324152,XM_{—}114229,gen.XM_{—}114229

FIG. 839: DNA324153,XM_{—}087122,gen.XM_{—}087122

FIG. 840: PRO80853

FIG. 841: DNA324154,XM_{—}018540,gen.XM_{—}018540

FIG. 842: DNA324155,XM_{—}087040,gen.XM_{—}087040

FIG. 843: DNA324156,NM_{—}032212,gen.NM_{—}032212

FIG. 844: PRO80856

FIG. 845: DNA324157,XM_{—}002217,gen.XM_{—}002217

FIG. 846: PRO80857

FIG. 847: DNA324158,NM_{—}000576,gen.NM_{—}000576

FIG. 848: PRO65

FIG. 849: DNA324159,XM_{—}086923,gen.XM_{—}086923

FIG. 850: DNA324160,XM_{—}086925,gen.XM_{—}086925

FIG. 851A-B: DNA324161,XM_{—}114266,gen.XM_{—}114266

FIG. 852: PRO80860

FIG. 853: DNA324162,XM_{—}002704,gen.XM_{—}002704

FIG. 854: DNAl94740,NM_{—}005291,gen.NM_{—}005291

FIG. 855: PRO24028

FIG. 856A-B: DNA324163,XM_{—}114267,gen.XM_{—}114267

FIG. 857: DNA324164,XM_{—}034952,gen.XM_{—}034952

FIG. 858: DNA324165,XM_{—}086950,gen.XM_{—}086950

FIG. 859A-B: DNA255531,NM_{—}017751,gen.NM_{—}017751

FIG. 860: PRO50596

FIG. 861: DNA324166,XM_{—}017698,gen.XM_{—}017698

FIG. 862: DNA324167,XM_{—}030529,gen.XM_{—}030529

FIG. 863: PRO80866

FIG. 864: DNA275240,NM_{—}005915,gen.NM_{—}005915

FIG. 865: PRO62927

FIG. 866: DNA324168,XM_{—}043173,gen.XM_{—}043173

FIG. 867: DNA324169,XM_{—}092489,gen.XM_{—}092489

FIG. 868: PRO80868

FIG. 869: DNA324170,XM_{—}115672,gen.XM_{—}115672

FIG. 870: PRO80869

FIG. 871: DNA324171,NM_{—}020548,gen.NM_{—}020548

FIG. 872: PRO60753

FIG. 873: DNA324172,XM_{—}037101,gen.XM_{—}037101

FIG. 874: PRO80870

FIG. 875: DNA324173,NM_{—}032390,gen.NM_{—}032390

FIG. 876: PRO80871

FIG. 877: DNA324174,XM_{—}002447,gen.XM_{—}002447

FIG. 878: DNA324175,NM_{—}033416,gen.NM_{—}033416

FIG. 879: PRO80873

FIG. 880: DNA324176,XM_{—}016288,gen.XM_{—}016288

FIG. 881: DNA272127,NM_{—}003937,gen.NM_{—}003937

FIG. 882: PRO60397

FIG. 883: DNA324177,XM_{—}030582,gen.XM_{—}030582

FIG. 884: PRO80875

FIG. 885: DNA324178,NM_{—}015702,gen.NM_{—}015702

FIG. 886: PRO80876

FIG. 887: DNA324179,NM_{—}016838,gen.NM_{—}016838

FIG. 888: PRO80877

FIG. 889: DNA324180,NM_{—}016839,gen.NM_{—}016839

FIG. 890: PRO80878

FIG. 891: DNA324181,XM_{—}087118,gen.XM_{—}087118

FIG. 892: PRO80879

FIG. 893: DNA324182,XM_{—}165998,gen.XM_{—}165998

FIG. 894: DNA324183,NM_{—}001935,gen.NM_{—}001935

FIG. 895: PRO80881

FIG. 896: DNA324184,NM_{—}020675,gen.NM_{—}020675

FIG. 897: PRO80882

FIG. 898: DNA88051,NM_{—}000079,gen.NM_{—}000079

FIG. 899: PRO2146

FIG. 900: DNA324185,XM_{—}166008,gen.XM_{—}166008

FIG. 901: DNA324186,XM_{—}087240,gen.XM_{—}087240

FIG. 902: PRO11403

FIG. 903: DNA324187,NM_{—}013341,gen.NM_{—}013341

FIG. 904: PRO80883

FIG. 905: DNA304805,NM_{—}031942,gen.NM_{—}031942

FIG. 906: PRO69531

FIG. 907: DNA324188,XM_{—}059465,gen.XM_{—}059465

FIG. 908: PRO80884

FIG. 909: DNA324189,XM_{—}015920,gen.XM_{—}015920

FIG. 910: DNA324190,XM_{—}166007,gen.XM_{—}166007

FIG. 911: DNA324191,XM_{—}015922,gen.XM_{—}015922

FIG. 912: DNA324192,XM_{—}087061,gen.XM_{—}087061

FIG. 913: PRO80888

FIG. 914: DNA324193,XM_{—}087062,gen.XM_{—}087062

FIG. 915: PRO80889

FIG. 916: DNA324194,NM_{—}001463,gen.NM_{—}001463

FIG. 917: PRO80890

FIG. 918: DNA324195,XM_{—}092158,gen.XM_{—}092158

FIG. 919: PRO80891

FIG. 920: DNA324196,XM_{—}059351,gen.XM_{—}059351

FIG. 921A-B: DNA324197,NM_{—}000090,gen.NM_{—}000090

FIG. 922: PRO2665

FIG. 923: DNA324198,NM_{—}014585,gen.NM_{—}014585

FIG. 924: PRO37675

FIG. 925: DNA324199,XM_{—}010778,gen.XM_{—}010778

FIG. 926: DNA324200,XM_{—}086961,gen.XM_{—}086961

FIG. 927: DNA324201,XM_{—}165994,gen.XM_{—}165994

FIG. 928: DNA324202,XM_{—}045170,gen.XM_{—}045170

FIG. 929: DNA324203,XM_{—}113390,gen.XM_{—}113390

FIG. 930: DNA299899,NM_{—}002157,gen.NM_{—}002157

FIG. 931: PRO62760

FIG. 932: DNA324204,XM_{—}087045,gen.XM_{—}087045

FIG. 933: DNA324205,XM_{—}086944,gen.XM_{—}086944

FIG. 934: DNA271608,NM_{—}014670,gen.NM_{—}014670

FIG. 935: PRO59895

FIG. 936: DNA324206,XM_{—}027963,gen.XM_{—}027963

FIG. 937: PRO80900

FIG. 938: DNA324207,XM_{—}010852,gen.XM_{—}010852

FIG. 939: PRO80901

FIG. 940: DNA324208,XM_{—}028034,gen.XM_{—}028034

FIG. 941: DNA324209,NM_{—}015934,gen.NM_{—}015934

FIG. 942: DNA324210,XM_{—}087028,gen.XM_{—}087028

FIG. 943: PRO80903

FIG. 944: DNA324211,XM_{—}092346,gen.XM_{—}092346

FIG. 945: PRO80904

FIG. 946: DNA324212,XM_{—}002669,gen.XM_{—}002669

FIG. 947: PRO80905

FIG. 948: DNA324213,NM_{—}021121,gen.NM_{—}021121

FIG. 949: PRO23124

FIG. 950: DNA324214,NM_{—}001959,gen.NM_{—}001959

FIG. 951: PRO23124

FIG. 952: DNA324215,XM_{—}030834,gen.XM_{—}030834

FIG. 953: PRO80906

FIG. 954A-C: DNA324216,XM_{—}055254,gen.XM_{—}055254

FIG. 955: DNA324217,NM_{—}004044,gen.NM_{—}004044

FIG. 956: PRO80908

FIG. 957: DNA324218,XM_{—}114298,gen.XM_{—}114298

FIG. 958: DNA324219,NM_{—}021141,gen.NM_{—}021141

FIG. 959: PRO59313

FIG. 960A-B: DNA324220,XM_{—}098048,gen.XM_{—}098048

FIG. 961: PRO80910

FIG. 962: DNA324221,XM_{—}098047,gen.XM_{—}098047

FIG. 963: PRO80911

FIG. 964: DNA324222,XM_{—}002636,gen.XM_{—}002636

FIG. 965: DNA324223,XM_{—}087181,gen.XM_{—}087181

FIG. 966: DNA324224,NM_{—}000998,gen.NM_{—}000998

FIG. 967: PRO10498

FIG. 968: DNA324225,XM_{—}059422,gen.XM_{—}059422

FIG. 969: PRO9984

FIG. 970: DNA324226,XM_{—}092545,gen.XM_{—}092545

FIG. 971: DNA324227,XM_{—}059461,gen.XM_{—}059461

FIG. 972: PRO80915

FIG. 973: DNA324228,NM_{—}018674,gen.NM_{—}018674

FIG. 974: PRO80916

FIG. 975: DNA324229,XM_{—}050962,gen.XM_{—}050962

FIG. 976: PRO80917

FIG. 977: DNA194827,NM_{—}012100,gen.NM_{—}012100

FIG. 978: PRO24091

FIG. 979: DNA324230,XM_{—}050638,gen.XM_{—}050638

FIG. 980A-B: DNA324231,NM_{—}002846,gen.NM_{—}002846

FIG. 981: PRO2610

FIG. 982: DNA324232,NM_{—}006000,gen.NM_{—}006000

FIG. 983: PRO26228

FIG. 984: DNA324233,XM_{—}050891,gen.XM_{—}050891

FIG. 985: DNA324234,XM_{—}087162,gen.XM_{—}087162

FIG. 986: DNA324235,XM_{—}058098,gen.XM_{—}058098

FIG. 987: PRO80920

FIG. 988: DNA324236,NM_{—}022453,gen.NM_{—}022453

FIG. 989: PRO80921

FIG. 990: DNA324237,NM_{—}032726,gen.NM_{—}032726

FIG. 991: PRO70675

FIG. 992: DNA324238,XM_{—}010866,gen.XM_{—}010866

FIG. 993: DNA324239,XM_{—}087166,gen.XM_{—}087166

FIG. 994: DNA254204,NM_{—}001087,gen.NM_{—}001087

FIG. 995: PRO49316

FIG. 996: DNA324240,NM_{—}005731,gen.NM_{—}005731

FIG. 997: PRO80924

FIG. 998: DNA189697,NM_{—}004846,gen.NM_{—}004846

FIG. 999: PRO23123

FIG. 1000: DNA324241,NM_{—}025202,gen.NM_{—}025202

FIG. 1001: PRO80925

FIG. 1002: DNA324242,XM_{—}115825,gen.XM_{—}115825

FIG. 1003: PRO80926

FIG. 1004: DNA324243,XM_{—}010858,gen.XM_{—}010858

FIG. 1005: PRO80927

FIG. 1006: DNA324244,XM_{—}002540,gen.XM_{—}002540

FIG. 1007: DNA324245,XM_{—}048690,gen.XM_{—}048690

FIG. 1008: PRO80929

FIG. 1009: DNA324246,NM_{—}030926,gen.NM_{—}030926

FIG. 1010: PRO80930

FIG. 1011: DNA324247,XM_{—}087218,gen.XM_{—}087218

FIG. 1012: DNA324248,NM_{—}004509,gen.NM_{—}004509

FIG. 1013: PRO80932

FIG. 1014: DNA324249,NM_{—}004510,gen.NM_{—}004510

FIG. 1015: PRO80933

FIG. 1016: DNA324250,NM_{—}080424,gen.NM_{—}080424

FIG. 1017: PRO80934

FIG. 1018: DNA324251,NM_{—}018410,gen.NM_{—}018410

FIG. 1019: PRO80935

FIG. 1020: DNA324252,NM_{—}017974,gen.NM_{—}017974

FIG. 1021: PRO80936

FIG. 1022A-B: DNA324253,XM_{—}096169,gen.XM_{—}096169

FIG. 1023: PRO80937

FIG. 1024: DNA150884,NM_{—}005855,gen.NM_{—}005855

FIG. 1025: PRO12520

FIG. 1026A-B: DNA324254,NM_{—}004735,gen.NM_{—}004735

FIG. 1027: PRO80938

FIG. 1028A-C: DNA324255,XM_{—}030203,gen.XM_{—}030203

FIG. 1029: DNA324256,XM_{—}059372,gen.XM_{—}059372

FIG. 1030: DNA324257,NM_{—}002712,gen.NM_{—}002712

FIG. 1031: PRO80941

FIG. 1032A-B: DNA324258,XM_{—}042326,gen.XM_{—}042326

FIG. 1033: PRO80942

FIG. 1034: DNA324259,NM_{—}004404,gen.NM_{—}004404

FIG. 1035: PRO80943

FIG. 1036: DNA324260,XM_{—}002742,gen.XM_{—}002742

FIG. 1037: DNA324261,NM_{—}138483,gen.NM_{—}138483

FIG. 1038: PRO80945

FIG. 1039: DNA324262,XM_{—}115706,gen.XM_{—}115706

FIG. 1040: DNA324263,XM_{—}115722,gen.XM_{—}115722

FIG. 1041: DNA324264,XM_{—}084141,gen.XM_{—}084141

FIG. 1042: DNA324265,XM_{—}005086,gen.XM_{—}005086

FIG. 1043: DNA324266,NM_{—}015453,gen.NM_{—}015453

FIG. 1044: PRO80949

FIG. 1045: DNA324267,NM_{—}022485,gen.NM_{—}022485

FIG. 1046: PRO80950

FIG. 1047A-B: DNA324268,XM_{—}054520,gen.XM_{—}054520

FIG. 1048: PRO80951

FIG. 1049: DNA324269,NM_{—}006354,gen.NM_{—}006354

FIG. 1050: PRO80952

FIG. 1051: DNA324270,NM_{—}133480,gen.NM_{—}133480

FIG. 1052: PRO80953

FIG. 1053: DNA324271,NM_{—}133481,gen.NM_{—}133481

FIG. 1054: PRO80954

FIG. 1055: DNA324272,NM_{—}005718,gen.NM_{—}005718

FIG. 1056: PRO80955

FIG. 1057: DNA324273,NM_{—}015644,gen.NM_{—}015644

FIG. 1058: PRO80956

FIG. 1059: DNA324274,XM_{—}059561,gen.XM_{—}059561

FIG. 1060: DNA324275,XM_{—}052310,gen.XM_{—}052310

FIG. 1061: PRO80958

FIG. 1062: DNA269910,NM_{—}006395,gen.NM_{—}006395

FIG. 1063: PRO58308

FIG. 1064: DNA324276,NM_{—}000994,gen.NM_{—}000994

FIG. 1065: PRO80959

FIG. 1066: DNA151017,NM_{—}004844,gen.NM_{—}004844

FIG. 1067: PRO12841

FIG. 1068: DNA324277,XM_{—}059557,gen.XM_{—}059557

FIG. 1069: PRO80960

FIG. 1070A-B: DNA324278,XM_{—}042860,gen.XM_{—}042860

FIG. 1071: PRO80961

FIG. 1072: DNA324279,XM_{—}042841,gen.XM_{—}042841

FIG. 1073: PRO80962

FIG. 1074: DNA324280,XM_{—}053712,gen.XM_{—}053712

FIG. 1075: DNA324281,XM_{—}087284,gen.XM_{—}087284

FIG. 1076: DNA324282,NM_{—}002948,gen.NM_{—}002948

FIG. 1077: PRO6360

FIG. 1078: DNA324283,XM_{—}053323,gen.XM_{—}053323

FIG. 1079A-B: DNA324284,NM_{—}001068,gen.NM_{—}001068

FIG. 1080: PRO80966

FIG. 1081: DNA252367,NM_{—}017801,gen.NM_{—}017801

FIG. 1082: PRO48357

FIG. 1083: DNA324285,XM_{—}093624,gen.XM_{—}093624

FIG. 1084: PRO80967

FIG. 1085: DNA324286,XM_{—}046401,gen.XM_{—}046401

FIG. 1086: DNA324287,NM_{—}022461,gen.NM_{—}022461

FIG. 1087: PRO80969

FIG. 1088: DNA324288,XM_{—}113410,gen.XM_{—}113410

FIG. 1089: DNA88100,NM_{—}000404,gen.NM_{—}000404

FIG. 1090: PRO2172

FIG. 1091: DNA324289,XM_{—}091076,gen.XM_{—}091076

FIG. 1092: PRO80970

FIG. 1093A-B: DNA271187,NM_{—}005109,gen.NM_{—}005109

FIG. 1094: PRO59504

FIG. 1095: DNA324290,NM_{—}002468,gen.NM_{—}002468

FIG. 1096: PRO36735

FIG. 1097: DNA269930,NM_{—}001607,gen.NM_{—}001607

FIG. 1098: PRO58328

FIG. 1099: DNA270401,NM_{—}003149,gen.NM_{—}003149

FIG. 1100: PRO58784

FIG. 1101: DNA324291,XM_{—}087370,gen.XM_{—}087370

FIG. 1102: PRO80971

FIG. 1103: DNA324292,XM_{—}098158,gen.XM_{—}098158

FIG. 1104: PRO80972

FIG. 1105: DNA324293,XM_{—}017364,gen.XM_{—}017364

FIG. 1106: DNA324294,XM_{—}087349,gen.XM_{—}087349

FIG. 1107: PRO80974

FIG. 1108: DNA226547,NM_{—}002295,gen.NM_{—}002295

FIG. 1109: PRO37010

FIG. 1110: DNA324295,NM_{—}003973,gen.NM_{—}003973

FIG. 1111: PRO80975

FIG. 1112: DNA324296,XM_{—}030417,gen.XM_{—}030417

FIG. 1113: DNA324297,NM_{—}020347,gen.NM_{—}020347

FIG. 1114: PRO80977

FIG. 1115: DNA324298,XM_{—}087346,gen.XM_{—}087346

FIG. 1116: PRO80978

FIG. 1117: DNA324299,XM_{—}096198,gen.XM_{—}096198

FIG. 1118: PRO80979

FIG. 1119: DNA324300,XM_{—}003222,gen.XM_{—}003222

FIG. 1120: DNA324301,XM_{—}087588,gen.XM_{—}087588

FIG. 1121: DNA324302,XM_{—}166011,gen.XM_{—}166011

FIG. 1122A-B: DNA324303,XM_{—}114364,gen.XM_{—}114364

FIG. 1123A-B: DNA324304,XM_{—}033294,gen.XM_{—}033294

FIG. 1124: PRO80983

FIG. 1125: DNA324305,NM_{—}138614,gen.NM_{—}138614

FIG. 1126: PRO80984

FIG. 1127: DNA324306,XM_{—}002899,gen.XM_{—}002899

FIG. 1128: DNA225910,NM_{—}004345,gen.NM_{—}004345

FIG. 1129: PRO36373

FIG. 1130: DNA324307,XM_{—}010953,gen.XM_{—}010953

FIG. 1131: DNA324308,XM_{—}051518,gen.XM_{—}051518

FIG. 1132A-D: DNA324309,NM_{—}001407,gen.NM_{—}001407

FIG. 1133: PRO50095

FIG. 1134: DNA324310,NM_{—}003365,gen.NM_{—}003365

FIG. 1135: PRO80988

FIG. 1136: DNA324311,XM_{—}003245,gen.XM_{—}003245

FIG. 1137: DNA324312,XM_{—}047561,gen.XM_{—}047561

FIG. 1138: PRO80990

FIG. 1139: DNA324313,XM_{—}116853,gen.XM_{—}116853

FIG. 1140A-B: DNA324314,XM_{—}113405,gen.XM_{—}113405

FIG. 1141: DNA324315,XM_{—}114323,gen.XM_{—}114323

FIG. 1142: PRO80993

FIG. 1143: DNA324316,XM_{—}002828,gen.XM_{—}002828

FIG. 1144: PRO80994

FIG. 1145: DNA150976,NM_{—}022171,gen.NM_{—}022171

FIG. 1146: PRO12565

FIG. 1147: DNA324317,XM_{—}041507,gen.XM_{—}041507

FIG. 1148: PRO71103

FIG. 1149: DNA103505,NM_{—}004636,gen.NM_{—}004636

FIG. 1150: PRO4832

FIG. 1151: DNA324318,NM_{—}006764,gen.NM_{—}006764

FIG. 1152: PRO80995

FIG. 1153: DNA150562,NM_{—}007275,gen.NM_{—}007275

FIG. 1154: PRO12779

FIG. 1155: DNA254582,NM_{—}004635,gen.NM_{—}004635

FIG. 1156: PRO49685

FIG. 1157: DNA324319,NM_{—}052859,gen.NM_{—}052859

FIG. 1158: PRO80996

FIG. 1159: DNA324320,NM_{—}001064,gen.NM_{—}001064

FIG. 1160: PRO80997

FIG. 1161: DNA324321,XM_{—}041211,gen.XM_{—}041211

FIG. 1162: DNA324322,XM_{—}003213,gen.XM_{—}003213

FIG. 1163A-C: DNA324323,XM_{—}037423,gen.XM_{—}037423

FIG. 1164: PRO80999

FIG. 1165A-B: DNA227307,NM_{—}007184,gen.NM_{—}007184

FIG. 1166: PRO37770

FIG. 1167: DNA324324,NM_{—}000688,gen.NM_{—}000688

FIG. 1168: PRO81000

FIG. 1169: DNA324325,XM_{—}067715,gen.XM_{—}067715

FIG. 1170: DNA324326,NM_{—}000992,gen.NM_{—}000992

FIG. 1171: PRO62153

FIG. 1172: DNA324327,NM_{—}000666,gen.NM_{—}000666

FIG. 1173: PRO81002

FIG. 1174: DNA324328,NM_{—}032750,gen.NM_{—}032750

FIG. 1175: PRO81003

FIG. 1176: DNA324329,NM_{—}033008,gen.NM_{—}033008

FIG. 1177: PRO81004

FIG. 1178: DNA324330,NM_{—}033010,gen.NM_{—}033010

FIG. 1179: PRO81005

FIG. 1180: DNA324331,NM_{—}020418,gen.NM_{—}020418

FIG. 1181: PRO81006

FIG. 1182: DNA273919,NM_{—}004704,gen.NM_{—}004704

FIG. 1183: PRO61870

FIG. 1184A-B: DNA324332,XM_{—}087448,gen.XM_{—}087448

FIG. 1185: PRO81007

FIG. 1186: DNA324333,XM_{—}002855,gen.XM_{—}002855

FIG. 1187: DNA324334,XM_{—}002854,gen.XM_{—}002854

FIG. 1188: DNA0,NM_{—}002854,gen.NM_{—}002854

FIG. 1189: PRO

FIG. 1190: DNA324335,XM_{—}096195,gen.XM_{—}096195

FIG. 1191: PRO81010

FIG. 1192: DNA324336,XM_{—}166015,gen.XM_{—}166015

FIG. 1193: DNA324337,XM_{—}113395,gen.XM_{—}113395

FIG. 1194: PRO81012

FIG. 1195: DNA269730,NM_{—}014814,gen.NM_{—}014814

FIG. 1196: PRO58140

FIG. 1197: DNA324338,XM_{—}036938,gen.XM_{—}036938

FIG. 1198: DNA324339,XM_{—}029369,gen.XM_{—}029369

FIG. 1199: DNA324340,XM_{—}076414,gen.XM_{—}076414

FIG. 1200: PRO81015

FIG. 1201: DNA324341,XM_{—}093546,gen.XM_{—}093546

FIG. 1202: DNA324342,XM_{—}113409,gen.XM_{—}113409

FIG. 1203: DNA324343,XM_{—}087268,gen.XM_{—}087268

FIG. 1204: DNA324344,XM_{—}116071,gen.XM_{—}116071

FIG. 1205: DNA324345,XM_{—}116072,gen.XM_{—}116072

FIG. 1206: DNA324346,NM_{—}000986,gen.NM_{—}000986

FIG. 1207: PRO10602

FIG. 1208: DNA324347,XM_{—}015462,gen.XM_{—}015462

FIG. 1209: DNA324348,XM_{—}167366,gen.XM_{—}167366

FIG. 1210: PRO81022

FIG. 1211: DNA324349,XM_{—}087331,gen.XM_{—}087331

FIG. 1212: PRO81023

FIG. 1213: DNA324350,XM_{—}039952,gen.XM_{—}039952

FIG. 1214: DNA324351,XM_{—}045290,gen.XM_{—}045290

FIG. 1215: PRO81025

FIG. 1216A-B: DNA324352,NM_{—}007085,gen.NM_{—}007085

FIG. 1217: PRO2077

FIG. 1218: DNA324353,NM_{—}004547,gen.NM_{—}004547

FIG. 1219: PRO81026

FIG. 1220: DNA324354,XM_{—}027161,gen.XM_{—}027161

FIG. 1221A-B: DNA324355,XM_{—}032269,gen.XM_{—}032269

FIG. 1222: PRO81028

FIG. 1223: DNA88547,NM_{—}006810,gen.NM_{—}006810

FIG. 1224: PRO2837

FIG. 1225: DNA324356,XM_{—}114301,gen.XM_{—}114301

FIG. 1226: PRO81029

FIG. 1227: DNA324357,XM_{—}098173,gen.XM_{—}098173

FIG. 1228: PRO81030

FIG. 1229: DNA324358,XM_{—}042618,gen.XM_{—}042618

FIG. 1230: PRO81031

FIG. 1231: DNA324359,XM_{—}084129,gen.XM_{—}084129

FIG. 1232: DNA324360,XM_{—}098154,gen.XM_{—}098154

FIG. 1233: PRO81033

FIG. 1234: D05524361,XM_{—}050552,gen.XM_{—}050552

FIG. 1235: DNA324362,NM_{—}032343,gen.NM_{—}032343

FIG. 1236: PRO81034

FIG. 1237: DNA324363,XM_{—}051264,gen.XM_{—}051264

FIG. 1238A-B: DNA324364,NM_{—}013336,gen.NM_{—}013336

FIG. 1239: PR01314

FIG. 1240: DNA324365,XM_{—}067264,gen.XM_{—}067264

FIG. 1241: PRO81036

FIG. 1242: DNA324366,XM_{—}114309,gen.XM_{—}114309

FIG. 1243: DNA324367,XM_{—}084111,gen.XM_{—}084111

FIG. 1244: DNA324368,XM_{—}113397,gen.XM_{—}113397

FIG. 1245: DNA324369,XM_{—}098111,gen.XM_{—}098111

FIG. 1246: DNA324370,NM_{—}004637,gen.NM_{—}004637

FIG. 1247: PRO81040

FIG. 1248: DNA324371,NM_{—}020701,gen.NM_{—}020701

FIG. 1249: PRO81041

FIG. 1250: DNA324372,NM_{—}003418,gen.NM_{—}003418

FIG. 1251: PRO81042

FIG. 1252: DNA324373,XM_{—}059583,gen.XM_{—}059583

FIG. 1253: PRO81043

FIG. 1254: DNA324374,XM_{—}113417,gen.XM_{—}113417

FIG. 1255: DNA324375,XM_{—}093487,gen.XM_{—}093487

FIG. 1256A-B: DNA324376,XM_{—}030812,gen.XM_{—}030812

FIG. 1257: PRO58177

FIG. 1258A-B: DNA324377,XM_{—}039805,gen.XM_{—}039805

FIG. 1259: PRO81046

FIG. 1260: DNA324378,NM_{—}000532,gen.NM_{—}000532

FIG. 1261: PRO81047

FIG. 1262: DNA324379,XM_{—}036118,gen.XM_{—}036118

FIG. 1263: DNA324380,XM_{—}084123,gen.XM_{—}084123

FIG. 1264: DNA324381,XM_{—}018149,gen.XM_{—}018149

FIG. 1265: DNA324382,XM_{—}087342,gen.XM_{—}087342

FIG. 1266: DNA324383,XM_{—}059516,gen.XM_{—}059516

FIG. 1267: DNA324384,XM_{—}087341,gen.XM_{—}087341

FIG. 1268: DNA324385,XM_{—}165451,gen.XM_{—}165451

FIG. 1269: PRO81053

FIG. 1270: DNA269858,NM_{—}004766,gen.NM_{—}004766

FIG. 1271: PRO58259

FIG. 1272: DNA324386,NM_{—}030921,gen.NM_{—}030921

FIG. 1273: PRO51109

FIG. 1274: DNA324387,XM_{—}002859,gen.XM_{—}002859

FIG. 1275: DNA324388,XM_{—}166014,gen.XM_{—}166014

FIG. 1276: DNA324389,NM_{—}013363,gen.NM_{—}013363

FIG. 1277: PRO287

FIG. 1278: DNA324390,XM_{—}058267,gen.XM_{—}058267

FIG. 1279: PRO81056

FIG. 1280A-B: DNA324391,NM_{—}032383,gen.NM_{—}032383

FIG. 1281: PRO81057

FIG. 1282: DNA324392,NM_{—}015472,gen.NM_{—}015472

FIG. 1283: PRO81058

FIG. 1284: DNA324393,NM_{—}014445,gen.NM_{—}014445

FIG. 1285: PRO11048

FIG. 1286: DNA324394,XM_{—}042168,gen.XM_{—}042168

FIG. 1287: PRO81059

FIG. 1288A-B: DNA324395,XM_{—}114356,gen.XM_{—}114356

FIG. 1289: DNA324396,XM_{—}105236,gen.XM_{—}105236

FIG. 1290: DNA324397,XM_{—}010978,gen.XM_{—}010978

FIG. 1291: DNA324398,XM_{—}017356,gen.XM_{—}017356

FIG. 1292A-B: DNA324399,XM_{—}039796,gen.XM_{—}39796

FIG. 1293: PRO81064

FIG. 1294: DNA324400,XM_{—}016334,gen.XM_{—}016334

FIG. 1295: DNA324401,XM_{—}116058,gen.XM_{—}116058

FIG. 1296: DNA324402,XM_{—}113408,gen.XM_{—}113408

FIG. 1297: DNA324403,NM_{—}002492,gen.NM_{—}002492

FIG. 1298: PRO81068

FIG. 1299: DNA324404,XM_{—}037381,gen.XM_{—}037381

FIG. 1300: DNA324405,XM_{—}037377,gen.XM_{—}037377

FIG. 1301: PRO69681

FIG. 1302A-B: DNA324406,XM_{—}087254,gen.XM_{—}087254

FIG. 1303: PRO81070

FIG. 1304: DNA324407,XM_{—}037600,gen.XM_{—}037600

FIG. 1305: PRO81071

FIG. 1306: DNA324408,NM_{—}018023,gen.NM_{—}018023

FIG. 1307: PRO81072

FIG. 1308: DNA324409,XM_{—}093423,gen.XM_{—}093423

FIG. 1309: PRO81073

FIG. 1310: DNA324410,XM_{—}029136,gen.XM_{—}029136

FIG. 1311: PRO81074

FIG. 1312: DNA324411,XM_{—}087322,gen.XM_{—}087322

FIG. 1313A-B: DNA324412,XM_{—}029132,gen.XM_{—}029132

FIG. 1314A-B: DNA324413,XM_{—}029104,gen.XM_{—}029104

FIG. 1315: DNA324414,XM_{—}084120,gen.XM_{—}084120

FIG. 1316: DNA254620,NM_{—}005787,gen.NM_{—}005787

FIG. 1317: PRO49722

FIG. 1318: DNA324415,NM_{—}032331,gen.NM_{—}032331

FIG. 1319: PRO81079

FIG. 1320: DNA324416,XM_{—}011074,gen.XM_{—}011074

FIG. 1321: PRO81080

FIG. 1322: DNA324417,XM_{—}087295,gen.XM_{—}087295

FIG. 1323: DNA324418,XM_{—}087289,gen.XM_{—}087289

FIG. 1324: PRO81082

FIG. 1325: DNA324419,XM_{—}105658,gen.XM_{—}105658

FIG. 1326: PRO81083

FIG. 1327: DNA89239,NM_{—}000893,gen.NM_{—}000893

FIG. 1328: PRO2906

FIG. 1329: DNA324420,XM_{—}113422,gen.XM_{—}113422

FIG. 1330: DNA225592,NM_{—}001622,gen.NM_{—}001622

FIG. 1331: PRO36055

FIG. 1332: DNA324421,XM_{—}005180,gen.XM_{—}005180

FIG. 1333: DNA324422,XM_{—}087392,gen.XM_{—}087392

FIG. 1334: PRO81086

FIG. 1335A-B: DNA272605,NM_{—}003722,gen.NM_{—}003722

FIG. 1336: PRO60741

FIG. 1337: DNA324423,XM_{—}117311,gen.XM_{—}117311

FIG. 1338: DNA324424,XM_{—}116034,gen.XM_{—}116034

FIG. 1339: PRO81088

FIG. 1340A-B: DNA324425,XM_{—}084110,gen.XM_{—}084110

FIG. 1341: DNA324426,XM_{—}038243,gen.XM_{—}038243

FIG. 1342: PRO81090

FIG. 1343: DNA324427,XM_{—}087359,gen.XM_{—}087359

FIG. 1344: DNA324428,XM_{—}114328,gen.XM_{—}114328

FIG. 1345: DNA324429,XM_{—}098109,gen.XM_{—}098109

FIG. 1346: PRO81093

FIG. 1347: DNA324430,XM_{—}087410,gen.XM_{—}087410

FIG. 1348: DNA324431,NM_{—}033316,gen.NM_{—}033316

FIG. 1349: PRO81095

FIG. 1350: DNA324432,XM_{—}166017,gen.XM_{—}166017

FIG. 1351: PRO81096

FIG. 1352: DNA79129,NM_{—}001647,gen.NM_{—}001647

FIG. 1353: PRO2551

FIG. 1354: DNA324433,NM_{—}032288,gen.NM_{—}032288

FIG. 1355: PRO81097

FIG. 1356: DNA324434,XM_{—}086228,gen.XM_{—}086228

FIG. 1357: PRO81098

FIG. 1358: DNA324435,XM_{—}087278,gen.XM_{—}087278

FIG. 1359: DNA324436,XM_{—}018523,gen.XM_{—}018523

FIG. 1360: DNA324437,XM_{—}087297,gen.XM_{—}087297

FIG. 1361: DNA324438,XM_{—}002255,gen.XM_{—}002255

FIG. 1362: PRO81102

FIG. 1363: DNA324439,XM_{—}053122,gen.XM_{—}053122

FIG. 1364: DNA324440,XM_{—}042695,gen.XM_{—}042695

FIG. 1365: DNA324441,XM_{—}011160,gen.XM_{—}011160

FIG. 1366: DNA324442,NM_{—}007100,gen.NM_{—}007100

FIG. 1367: PRO81106

FIG. 1368: DNA139747,NM_{—}002477,gen.NM_{—}002477

FIG. 1369: PRO9785

FIG. 1370: DNA253804,NM_{—}032219,gen.NM_{—}032219

FIG. 1371: PRO49209

FIG. 1372: DNA324443,NM_{—}138385,gen.NM_{—}138385

FIG. 1373: PRO81107

FIG. 1374: DNA324444,NM_{—}006342,gen.NM_{—}006342

FIG. 1375: PRO81108

FIG. 1376A-C: DNA324445,NM_{—}133330,gen.NM_{—}133330

FIG. 1377: PRO81109

FIG. 1378A-C: DNA324446,NM_{—}014919,gen.NM_{—}014919

FIG. 1379: PRO81110

FIG. 1380A-C: DNA324447,NM_{—}133332,gen.NM_{—}133332

FIG. 1381: PRO81111

FIG. 1382: DNA324448,NM_{—}005663,gen.NM_{—}005663

FIG. 1383: PRO81112

FIG. 1384A-B: DNA324449,XM_{—}098248,gen.XM_{—}098248

FIG. 1385: PRO81113

FIG. 1386: DNA270615,NM_{—}002938,gen.NM_{—}002938

FIG. 1387: PRO58986

FIG. 1388A-B: DNA324450,NM_{—}014190,gen.NM_{—}014190

FIG. 1389: PRO81114

FIG. 1390A-B: DNA324451,NM_{—}014189,gen.NM_{—}014189

FIG. 1391: PRO81115

FIG. 1392: DNA324452,XM_{—}035572,gen.XM_{—}035572

FIG. 1393: PRO81116

FIG. 1394A-B: DNA324453,NM_{—}014556,gen.NM_{—}014556

FIG. 1395: PRO81117

FIG. 1396: DNA324454,NM_{—}001313,gen.NM_{—}001313

FIG. 1397: PRO60542

FIG. 1398A-B: DNA324455,XM_{—}052626,gen.XM_{—}052626

FIG. 1399: PRO81118

FIG. 1400: DNA324456,NM_{—}016930,gen.NM_{—}016930

FIG. 1401: PRO81119

FIG. 1402: DNA324457,XM_{—}035824,gen.XM_{—}035824

FIG. 1403: PRO81120

FIG. 1404: DNA324458,NM_{—}033296,gen.NM_{—}033296

FIG. 1405: PRO81121

FIG. 1406: DNA324459,NM_{—}138699,gen.NM_{—}138699

FIG. 1407: PRO81122

FIG. 1408: DNA324460,XM_{—}116285,gen.XM_{—}116285

FIG. 1409: PRO81123

FIG. 1410: DNA324461,XM_{—}041221,gen.XM_{—}041221

FIG. 1411: PRO81124

FIG. 1412: DNA324462,XM_{—}117351,gen.XM_{—}117351

FIG. 1413: DNA324463,XM_{—}039165,gen.XM_{—}039165

FIG. 1414: DNA324464,NM_{—}025205,gen.NM_{—}025205

FIG. 1415: PRO81127

FIG. 1416: DNA324465,XM_{—}039173,gen.XM_{—}039173

FIG. 1417: DNA324466,XM_{—}039176,gen.XM_{—}039176

FIG. 1418: DNA324467,XM_{—}087583,gen.XM_{—}087583

FIG. 1419: DNA324468,NM_{—}017491,gen.NM_{—}017491

FIG. 1420: PRO12077

FIG. 1421: DNA324469,NM_{—}005112,gen.NM_{—}005112

FIG. 1422: PRO81131

FIG. 1423: DNA324470,XM_{—}011129,gen.XM_{—}011129

FIG. 1424A-B: DNA324471,XM_{—}052530,gen.XM_{—}052530

FIG. 1425: DNA324472,NM_{—}000661,gen.NM_{—}000661

FIG. 1426: PRO81134

FIG. 1427A-B: DNA324473,NM_{—}002913,gen.NM_{—}002913

FIG. 1428: PRO81135

FIG. 1429A-B: DNA324474,XM_{—}047477,gen.XM_{—}047477

FIG. 1430: DNA324475,NM_{—}004181,gen.NM_{—}004181

FIG. 1431: PRO81137

FIG. 1432: DNA324476,XM_{—}003435,gen.XM_{—}003435

FIG. 1433: DNA324478,XM_{—}010941,gen.XM_{—}010941

FIG. 1434: DNA324479,XM_{—}059593,gen.XM_{—}059593

FIG. 1435: DNA324480,NM_{—}001553,gen.NM_{—}001553

FIG. 1436: PRO81141

FIG. 1437: DNA257511,NM_{—}032313,gen.NM_{—}032313

FIG. 1438: PRO52083

FIG. 1439: DNA324481,XM_{—}071623,gen.XM_{—}071623

FIG. 1440A-B: DNA324482,XM_{—}036002,gen.XM_{—}036002

FIG. 1441: DNA324483,XM_{—}058927,gen.XM_{—}058927

FIG. 1442: DNA324484,XM_{—}059628,gen.XM_{—}059628

FIG. 1443: DNA324485,XM_{—}046057,gen.XM_{—}046057

FIG. 1444: PRO81146

FIG. 1445: DNA324486,XM_{—}031320,gen.XM_{—}031320

FIG. 1446: DNA225919,NM_{—}001134,gen.NM_{—}001134

FIG. 1447: PRO36382

FIG. 1448A-B: DNA324487,XM_{—}03511,gen.XM_{—}003511

FIG. 1449: DNA324488,NM_{—}006835,gen.NM_{—}006835

FIG. 1450: PRO4605

FIG. 1451: DNA324489,XM_{—}003305,gen.XM_{—}003305

FIG. 1452: DNA324490,XM_{—}113425,gen.XM_{—}113425

FIG. 1453: DNA324491,XM_{—}001389,gen.XM_{—}001389

FIG. 1454: PRO81148

FIG. 1455: DNA324492,XM_{—}087527,gen.XM_{—}087527

FIG. 1456: DNA324493,XM_{—}035986,gen.XM_{—}035986

FIG. 1457A-B: DNA324494,NM_{—}014933,gen.NM_{—}014933

FIG. 1458: PRO81150

FIG. 1459: DNA290585,NM_{—}000582,gen.NM_{—}000582

FIG. 1460: PRO70536

FIG. 1461: DNA324495,XM_{—}055551,gen.XM_{—}055551

FIG. 1462: PRO81151

FIG. 1463: DNA324496,XM_{—}087498,gen.XM_{—}087498

FIG. 1464: DNA324497,XM_{—}096203,gen.XM_{—}096203

FIG. 1465: DNA324498,XM_{—}084158,gen.XM_{—}084158

FIG. 1466: DNA324499,XM_{—}034710,gen.XM_{—}034710

FIG. 1467: PRO81156

FIG. 1468: DNA324500,XM_{—}034713,gen.XM_{—}034713

FIG. 1469: DNA324501,XM_{—}059633,gen.XM_{—}059633

FIG. 1470: DNA324502,XM_{—}114426,gen.XM_{—}114426

FIG. 1471: DNA324503,XM_{—}056957,gen.XM_{—}056957

FIG. 1472: DNA324504,XM_{—}088472,gen.XM_{—}088472

FIG. 1473: DNA324505,XM_{—}114424,gen.XM_{—}114424

FIG. 1474A-B: DNA324506,XM_{—}042301,gen.XM_{—}042301

FIG. 1475: PRO81163

FIG. 1476: DNA324507,XM_{—}017925,gen.XM_{—}017925

FIG. 1477: DNA324508,XM_{—}052336,gen.XM_{—}052336

FIG. 1478: DNA324509,NM_{—}002106,gen.NM_{—}002106

FIG. 1479: PRO10297

FIG. 1480: DNA324510,XM_{—}085068,gen.XM_{—}085068

FIG. 1481: PRO81166

FIG. 1482: DNA324511,XM_{—}165473,gen.XM_{—}165473

FIG. 1483: DNA324512,XM_{—}087514,gen.XM_{—}087514

FIG. 1484: DNA324513,XM_{—}116247,gen.XM_{—}116247

FIG. 1485: DNA324514,NM_{—}002358,gen.NM_{—}002358

FIG. 1486: PRO81169

FIG. 1487: DNA324515,XM_{—}050200,gen.XM_{—}050200

FIG. 1488: PRO81170

FIG. 1489: DNA225584,NM_{—}001154,gen.NM_{—}001154

FIG. 1490: PRO36047

FIG. 1491: DNA324516,NM_{—}024900,gen.NM_{—}024900

FIG. 1492: PRO81171

FIG. 1493: DNA324517,XM_{—}040752,gen.XM_{—}040752

FIG. 1494: DNA324518,NM_{—}002413,gen.NM_{—}002413

FIG. 1495: PRO60956

FIG. 1496: DNA324519,XM_{—}114401,gen.XM_{—}114401

FIG. 1497: DNA324520,XM_{—}068164,gen.XM_{—}068164

FIG. 1498: PRO81174

FIG. 1499: DNA324521,XM_{—}060067,gen.XM_{—}060067

FIG. 1500: DNA324522,XM_{—}003555,gen.XM_{—}003555

FIG. 1501: PRO81176

FIG. 1502: DNA324523,XM_{—}034321,gen.XM_{—}034321

FIG. 1503: PRO81177

FIG. 1504: DNA324524,NM_{—}006439,gen.NM_{—}006439

FIG. 1505: PRO81178

FIG. 1506: DNA324525,NM_{—}001006,gen.NM_{—}001006

FIG. 1507: PRO81179

FIG. 1508: DNA227575,NM_{—}005141,gen.NM_{—}005141

FIG. 1509: PRO38038

FIG. 1510: DNA324526,XM_{—}114368,gen.XM_{—}114368

FIG. 1511A-B: DNA225920,NM_{—}000508,gen.NM_{—}000508

FIG. 1512: PRO36383

FIG. 1513: DNA324527,NM_{—}021871,gen.NM_{—}021871

FIG. 1514: PRO81181

FIG. 1515: DNA225921,NM_{—}000509,gen.NM_{—}000509

FIG. 1516: PRO36384

FIG. 1517: DNA324528,NM_{—}021870,gen.NM_{—}021870

FIG. 1518: PRO81182

FIG. 1519: DNA324529,XM_{—}059623,gen.XM_{—}059623

FIG. 1520: DNA324530,XM_{—}106246,gen.XM_{—}106246

FIG. 1521: PRO81184

FIG. 1522: DNA324531,NM_{—}002129,gen.NM_{—}002129

FIG. 1523: PRO81185

FIG. 1524: DNA324532,XM_{—}040321,gen.XM_{—}040321

FIG. 1525: DNA324533,XM_{—}015563,gen.XM_{—}015563

FIG. 1526: DNA324534,NM_{—}024748,gen.NM_{—}024748

FIG. 1527: PRO81188

FIG. 1528: DNA324535,XM_{—}165470,gen.XM_{—}165470

FIG. 1529: PRO81189

FIG. 1530A-E: DNA324536,XM_{—}003477,gen.XM_{—}003477

FIG. 1531: DNA324537,XM_{—}165465,gen.XM_{—}165465

FIG. 1532: DNA324538,XM_{—}116204,gen.XM_{—}116204

FIG. 1533: DNA324539,XM_{—}116205,gen.XM_{—}116205

FIG. 1534: DNA324540,XM_{—}098405,gen.XM_{—}098405

FIG. 1535: DNA324541,XM_{—}052313,gen.XM_{—}052313

FIG. 1536: PRO81195

FIG. 1537: DNA324542,XM_{—}087659,gen.XM_{—}087659

FIG. 1538: PRO81196

FIG. 1539: DNA324543,XM_{—}029096,gen.XM_{—}029096

FIG. 1540: DNA324544,XM_{—}003825,gen.XM_{—}003825

FIG. 1541: DNA324545,XM_{—}057994,gen.XM_{—}057994

FIG. 1542: PRO81199

FIG. 1543: DNA324546,XM_{—}087686,gen.XM_{—}087686

FIG. 1544: DNA324547,XM_{—}017641,gen.XM_{—}017641

FIG. 1545: DNA324548,NM_{—}030782,gen.NM_{—}030782

FIG. 1546: PRO81202

FIG. 1547: DNA324549,XM_{—}084168,gen.XM_{—}084168

FIG. 1548: DNA324550,XM_{—}057492,gen.XM_{—}057492

FIG. 1549: DNA324551,XM_{—}087597,gen.XM_{—}087597

FIG. 1550: DNA324552,XM_{—}087601,gen.XM_{—}087601

FIG. 1551: DNA324554,XM_{—}087599,gen.XM_{—}087599

FIG. 1552: DNA324555,XM_{—}114435,gen.XM_{—}114435

FIG. 1553: DNA324556,XM_{—}087600,gen.XM_{—}087600

FIG. 1554: DNA324557,XM_{—}016170,gen.XM_{—}016170

FIG. 1555: DNA324558,XM_{—}114434,gen.XM_{—}114434

FIG. 1556: DNA324559,XM_{—}113452,gen.XM_{—}113452

FIG. 1557: DNA324560,XM_{—}071580,gen.XM_{—}071580

FIG. 1558: PRO81213

FIG. 1559: DNA324561,XM_{—}087713,gen.XM_{—}087713

FIG. 1560: PRO81214

FIG. 1561: DNA324562,XM_{—}094440,gen.XM_{—}094440

FIG. 1562: DNA324563,XM_{—}106739,gen.XM_{—}106739

FIG. 1563: PRO81216

FIG. 1564: DNA324564,XM_{—}087614,gen.XM_{—}087614

FIG. 1565: DNA324565,XM_{—}004009,gen.XM_{—}004009

FIG. 1566: PRO81219

FIG. 1567: DNA324566,XM_{—}114437,gen.XM_{—}114437

FIG. 1568: DNA324567,XM_{—}043771,gen.XM_{—}043771

FIG. 1569: PRO81221

FIG. 1570: DNA324568,NM_{—}000997,gen.NM_{—}000997

FIG. 1571: PRO11077

FIG. 1572: DNA324569,XM_{—}003869,gen.XM_{—}003869

FIG. 1573: DNA227173,NM_{—}001465,gen.NM_{—}001465

FIG. 1574: PRO37636

FIG. 1575: DNA324570,NM_{—}018034,gen.NM_{—}018034

FIG. 1576: PRO81223

FIG. 1577: DNA324571,NM_{—}032637,gen.NM_{—}032637

FIG. 1578: PRO81224

FIG. 1579: DNA324572,NM_{—}005983,gen.NM_{—}005983

FIG. 1580: PRO81225

FIG. 1581A-B: DNA324573,XM_{—}003896,gen.XM_{—}003896

FIG. 1582: DNA287282,NM_{—}002130,gen.NM_{—}002130

FIG. 1583: PRO69554

FIG. 1584: DNA324574,XM_{—}114442,gen.XM_{—}114442

FIG. 1585: PRO81227

FIG. 1586: DNA324575,XM_{—}114439,gen.XM_{—}114439

FIG. 1587: DNA324576,XM_{—}114440,gen.XM_{—}114440

FIG. 1588A-B: DNA324577,XM_{—}032902,gen.XM_{—}032902

FIG. 1589: PRO81230

FIG. 1590: DNA324578,XM_{—}032895,gen.XM_{—}032895

FIG. 1591: DNA324579,XM_{—}084179,gen.XM_{—}084179

FIG. 1592: DNA324580,XM_{—}041712,gen.XM_{—}041712

FIG. 1593: DNA324581,XM_{—}116439,gen.XM_{—}116439

FIG. 1594: PRO81234

FIG. 1595: DNA324582,XM_{—}087611,gen.XM_{—}087611

FIG. 1596: DNA324583,XM_{—}059653,gen.XM_{—}059653

FIG. 1597: DNA324584,XM_{—}087610,gen.XM_{—}087610

FIG. 1598: DNA288259,NM_{—}031966,gen.NM_{—}031966

FIG. 1599: PRO4676

FIG. 1600: DNA324585,XM_{—}042025,gen.XM_{—}042025

FIG. 1601: PRO81238

FIG. 1602: DNA324586,NM_{—}005713,gen.NM_{—}005713

FIG. 1603: PRO81239

FIG. 1604: DNA324587,XM_{—}059709,gen.XM_{—}059709

FIG. 1605: PRO81240

FIG. 1606: DNA324588,XM_{—}116447,gen.XM_{—}116447

FIG. 1607: PRO81241

FIG. 1608: DNA324589,XM_{—}037260,gen.XM_{—}037260

FIG. 1609: DNA324590,XM_{—}098351,gen.XM_{—}098351

FIG. 1610: DNA324591,XM_{—}098354,gen.XM_{—}098354

FIG. 1611: DNA324592,XM_{—}098352,gen.XM_{—}098352

FIG. 1612: DNA324593,XM_{—}166037,gen.XM_{—}166037

FIG. 1613: PRO81246

FIG. 1614: DNA324594,XM_{—}041694,gen.XM_{—}041694

FIG. 1615: DNA324595,XM_{—}165488,gen.XM_{—}165488

FIG. 1616: PRO81248

FIG. 1617: DNA324596,XM_{—}059669,gen.XM_{—}059669

FIG. 1618: PRO81249

FIG. 1619: DNA324597,XM_{—}027964,gen.XM_{—}027964

FIG. 1620: PRO81250

FIG. 1621: DNA324598,XM_{—}088020,gen.XM_{—}088020

FIG. 1622: DNA324599,XM_{—}117387,gen.XM_{—}117387

FIG. 1623: DNA324600,XM_{—}114469,gen.XM_{—}114469

FIG. 1624: DNA324601,NM_{—}001207,gen.NM_{—}001207

FIG. 1625: PRO22771

FIG. 1626A-B: DNA324602,XM_{—}032553,gen.XM_{—}032553

FIG. 1627: DNA254147,NM_{—}000521,gen.NM_{—}000521

FIG. 1628: PRO49262

FIG. 1629: DNA324603,NM_{—}031482,gen.NM_{—}031482

FIG. 1630: PRO81254

FIG. 1631: DNA324604,XM_{—}087790,gen.XM_{—}087790

FIG. 1666: DNA324622,XM_{—}003830,gen.XM_{—}003830

FIG. 1632: DNA324605,NM_{—}001025,gen.NM_{—}001025

FIG. 1667: PRO81269

FIG. 1668: DNA324623,XM_{—}037002,gen.XM_{—}037002

FIG. 1633: PRO10685

FIG. 1634: DNA324606,XM_{—}098362,gen.XM_{—}098362

FIG. 1669: DNA324624,XM_{—}166026,gen.XM_{—}166026

FIG. 1635: PRO81256

FIG. 1670: DNA324625,XM_{—}041059,gen.XM_{—}041059

FIG. 1636: DNA324607,NM_{—}003401,gen.NM_{—}003401

FIG. 1671: DNA83020,NM_{—}000358,gen.NM_{—}000358

FIG. 1637: PRO70327

FIG. 1638: DNA290231,NM_{—}022550,gen.NM_{—}022550

FIG. 1672: PRO2561

FIG. 1673: DNA324626,NM_{—}003687,gen NM_{—}003687

FIG. 1639: PRO70327

FIG. 1640: DNA324608,XM_{—}017857,gen.XM_{—}017857

FIG. 1674: PRO81272

FIG. 1675: DNA324627,XM_{—}034862,gen.XM_{—}034862

FIG. 1641: DNA324609,XM_{—}117398,gen.XM_{—}117398

FIG. 1676: PRO34544

FIG. 1642A-B: DNA257253,NM_{—}032280,gen.NM_{—}032280

FIG. 1677: DNA103380,NM_{—}003374,gen.NM_{—}003374

FIG. 1643: PRO51851

FIG. 1678: PRO4710

FIG. 1644: DNA324610,XM_{—}003771,gen.XM_{—}003771

FIG. 1679: DNA324628,XM_{—}017474,gen.XM_{—}017474

FIG. 1645: PRO81259

FIG. 1680: PRO63082

FIG. 1646A-B: DNA269816,NM_{—}002397,gen.NM_{—}002397

FIG. 1681A-B: DNA324629,NM_{—}014829,gen.NM_{—}014829

FIG. 1647: PRO58219

FIG. 1682: PRO81273

FIG. 1648: DNA324611,XM_{—}116427,gen.XM_{—}116427

FIG. 1683A-B: DNA324630,XM_{—}114482,gen.XM_{—}114482

FIG. 1649: PRO81260

FIG. 1684: PRO81274

FIG. 1650: DNA324612,NM_{—}004772,gen.NM_{—}004772

FIG. 1685: DNA324631,NM_{—}004893,gen.NM_{—}004893

FIG. 1651: PRO81261

FIG. 1686: PRO81275

FIG. 1652: DNA324613,XM_{—}016674,gen.XM_{—}016674

FIG. 1687: DNA269809,NM_{—}006805,gen.NM_{—}006805

FIG. 1653: PRO81262

FIG. 1688: PRO58213

FIG. 1654: DNA324614,XM_{—}113463,gen.XM_{—}113463

FIG. 1689: DNA226872,NM_{—}001964,gen.NM_{—}001964

FIG. 1655: DNA324615,XM_{—}034744,gen.XM_{—}034744

FIG. 1690: PRO37335

FIG. 1691: DNA324632,XM_{—}116307,gen.XM_{—}116307

FIG. 1656: DNA324616,XM_{—}087745,gen.XM_{—}087745

FIG. 1692: PRO81276

FIG. 1657: PRO81264

FIG. 1693: DNA324633,NM_{—}004134,gen.NM_{—}004134

FIG. 1658: DNA324617,XM_{—}018473,gen.XM_{—}018473

FIG. 1694: PRO81277

FIG. 1659: PRO81265

FIG. 1695: DNA324634,XM_{—}038221,gen.XM_{—}038221

FIG. 1660: DNA324618,XM_{—}087635,gen.XM_{—}087635

FIG. 1696: PRO81278

FIG. 1661: PRO81266

FIG. 1697: DNA271931,NM_{—}005754,gen.NM_{—}005754

FIG. 1662: DNA324619,XM_{—}087637,gen.XM_{—}087637

FIG. 1698: PRO60207

FIG. 1663: DNA324620,XM_{—}166027,gen.XM_{—}166027

FIG. 1699: DNA324635,XM_{—}003841,gen.XM_{—}003841

FIG. 1664: DNA324621,NM_{—}014035,gen.NM_{—}014035

FIG. 1700: DNA324636,XM_{—}032759,gen.XM_{—}032759

FIG. 1665: PRO1285

FIG. 1701: DNA324637,XM_{—}017591,gen.XM_{—}017591

FIG. 1702: DNA324638,NM_{—}006058,gen.NM_{—}006058

FIG. 1703: PRO81280

FIG. 1704: DNA324639,NM_{—}002084,gen.NM_{—}002084

FIG. 1705: PRO81281

FIG. 1706: DNA324640,NM_{—}018047,gen.NM_{—}018047

FIG. 1707: PRO81282

FIG. 1708: DNA324641,NM_{—}005617,gen.NM_{—}005617

FIG. 1709: PRO10849

FIG. 1710: DNA324642,XM_{—}003937,gen.XM_{—}003937

FIG. 1711: DNA324643,XM_{—}087621,gen.XM_{—}087621

FIG. 1712A-B: DNA324644,XM_{—}003789,gen.XM_{—}003789

FIG. 1713: DNA324645,XM_{—}087652,gen.XM_{—}087652

FIG. 1714: DNA324646,XM_{—}068853,gen.XM_{—}068853

FIG. 1715: PRO81286

FIG. 1716: DNA324647,XM_{—}116465,gen.XM_{—}116465

FIG. 1717: PRO81287

FIG. 1718: DNA302020,NM_{—}005573,gen.NM_{—}005573

FIG. 1719: PRO70993

FIG. 1720: DNA324648,XM_{—}113467,gen.XM_{—}113467

FIG. 1721: DNA271626,NM_{—}014773,gen.NM_{—}014773

FIG. 1722: PRO59913

FIG. 1723A-B: DNA324649,XM_{—}056315,gen.XM_{—}056315

FIG. 1724: DNA324650,NM_{—}024668,gen.NM_{—}024668

FIG. 1725: PRO81289

FIG. 1726: DNA324651,NM_{—}080670,gen.NM_{—}080670

FIG. 1727: PRO81290

FIG. 1728A-B: DNA324652,NM_{—}002588,gen.NM_{—}002588

FIG. 1729: PRO81291

FIG. 1730A-B: DNA324653,NM_{—}003735,gen.NM_{—}003735

FIG. 1731: PRO81292

FIG. 1732A-B: DNA150679,NM_{—}003736,gen.NM_{—}003736

FIG. 1733: PRO12416

FIG. 1734A-B: DNA324654,NM_{—}018912,gen.NM_{—}018912

FIG. 1735: PRO36058

FIG. 1736A-B: DNA324655,NM_{—}018913,gen.NM_{—}018913

FIG. 1737: PRO81293

FIG. 1738A-B: DNA324656,NM_{—}018914,gen.NM_{—}018914

FIG. 1739: PRO81294

FIG. 1740A-B: DNA324657,NM_{—}018915,gen.NM_{—}018915

FIG. 1741: PRO36020

FIG. 1742A-B: DNA324658,NM_{—}018916,gen.NM_{—}018916

FIG. 1743: PRO81295

FIG. 1744A-B: DNA324659,NM_{—}018917,gen.NM_{—}018917

FIG. 1745: PRO81296

FIG. 1746A-B: DNA324660,NM_{—}018918,gen.NM_{—}018918

FIG. 1747: PRO81297

FIG. 1748A-B: DNA324661,NM_{—}018919,gen.NM_{—}018919

FIG. 1749: PRO81298

FIG. 1750A-B: DNA324662,NM_{—}018920,gen.NM_{—}018920

FIG. 1751: PRO81299

FIG. 1752A-B: DNA324663,NM_{—}018921,gen.NM_{—}018921

FIG. 1753: PRO81300

FIG. 1754A-B: DNA324664,NM_{—}018922,gen.NM_{—}018922

FIG. 1755: PRO81301

FIG. 1756A-B: DNA324665,NM_{—}018923,gen.NM_{—}018923

FIG. 1757: PRO81302

FIG. 1758A-B: DNA324666,NM_{—}018924,gen.NM_{—}018924

FIG. 1759: PRO81303

FIG. 1760A-B: DNA324667,NM_{—}018925,gen.NM_{—}018925

FIG. 1761: PRO81304

FIG. 1762A-B: DNA324668,NM_{—}018926,gen.NM_{—}018926

FIG. 1763: PRO81305

FIG. 1764A-B: DNA324669,NM_{—}018927,gen.NM_{—}018927

FIG. 1765: PRO37091

FIG. 1766A-B: DNA324670,NM_{—}018928,gen.NM_{—}018928

FIG. 1767: PRO81306

FIG. 1768A-B: DNA324671,NM_{—}018929,gen.NM_{—}018929

FIG. 1769: PRO81307

FIG. 1770A-B: DNA324672,NM_{—}032088,gen.NM_{—}032088

FIG. 1771: PRO81308

FIG. 1772A-B: DNA324673,NM_{—}032092,gen.NM_{—}032092

FIG. 1773: PRO81309

FIG. 1774: DNA324674,NM_{—}032403,gen.NM_{—}032403

FIG. 1775: PRO81310

FIG. 1776: DNA324675,NM_{—}032402,gen.NM_{—}032402

FIG. 1777: PRO81311

FIG. 1778: DNA324676,XM_{—}098387,gen.XM_{—}098387

FIG. 1779: DNA324677,NM_{—}002109,gen.NM_{—}002109

FIG. 1780: PRO4908

FIG. 1781: DNA324678,XM_{—}084180,gen.XM_{—}084180

FIG. 1782: PRO81313

FIG. 1783: DNA324679,XM_{—}039975,gen.XM_{—}039975

FIG. 1784: PRO81314

FIG. 1785: DNA324680,NM_{—}033551,gen.NM_{—}033551

FIG. 1786: PRO81315

FIG. 1787: DNA324681,NM_{—}004821,gen.NM_{—}004821

FIG. 1788: PRO81316

FIG. 1789: DNA324682,XM_{—}068395,gen.XM_{—}068395

FIG. 1790: PRO81317

FIG. 1791: DNA226418,NM_{—}004060,gen.NM_{—}004060

FIG. 1792: PRO36881

FIG. 1793A-B: DNA324683,XM_{—}56963,gen.XM_{—}056963

FIG. 1794: PRO81318

FIG. 1795: DNA324684,NM_{—}004219,gen.NM_{—}004219

FIG. 1796: PRO81319

FIG. 1797: DNA324685,XM_{—}094243,gen.XM_{—}094243

FIG. 1798A-B: DNA324686,XM_{—}047964,gen.XM_{—}047964

FIG. 1799: DNA324687,XM_{—}016345,gen.XM_{—}016345

FIG. 1800: DNA324688,NM_{—}002887,gen.NM_{—}002887

FIG. 1801: PRO81323

FIG. 1802: DNA324689,XM_{—}166029,gen.XM_{—}166029

FIG. 1803: DNA324690,NM_{—}002520,gen.NM_{—}002520

FIG. 1804: PRO58993

FIG. 1805: DNA324691,XM_{—}043340,gen.XM_{—}043340

FIG. 1806: PRO81325

FIG. 1807: DNA324692,XM_{—}116340,gen.XM_{—}116340

FIG. 1808A-B: DNA324693,XM_{—}043388,gen.XM_{—}043388

FIG. 1809: PRO81327

FIG. 1810: DNA324694,XM_{—}116856,gen.XM_{—}116856

FIG. 1811: DNA324695,XM_{—}003716,gen.XM_{—}003716

FIG. 1812: DNA227320,NM_{—}003714,gen.NM_{—}003714

FIG. 1813: PRO37783

FIG. 1814: DNA324696,NM_{—}032361,gen.NM_{—}032361

FIG. 1815: PRO81330

FIG. 1816: DNA324697,XM_{—}087773,gen.XM_{—}087773

FIG. 1817: DNA324698,XM_{—}114457,gen.XM_{—}114457

FIG. 1818: DNA324699,XM_{—}165483,gen.XM_{—}165483

FIG. 1819: DNA324700,XM_{—}114453,gen.XM_{—}114453

FIG. 1820: DNA324701,XM_{—}165484,gen.XM_{—}165484

FIG. 1821: DNA324702,XM_{—}030771,gen.XM_{—}030771

FIG. 1822: PRO19615

FIG. 1823: DNA324703,XM_{—}030777,gen.XM_{—}030777

FIG. 1824: DNA324704,XM_{—}030782,gen.XM_{—}030782

FIG. 1825: PRO81336

FIG. 1826: DNA324705,NM_{—}030567,gen.NM_{—}030567

FIG. 1827: PRO81337

FIG. 1828: DNA225909,NM_{—}000505,gen.NM_{—}000505

FIG. 1829: PRO36372

FIG. 1830: DNA274206,NM_{—}006816,gen.NM_{—}006816

FIG. 1831: PRO62135

FIG. 1832: DNA324706,NM_{—}031300,gen.NM_{—}031300

FIG. 1833: PRO81338

FIG. 1834: DNA324707,NM_{—}013237,gen.NM_{—}013237

FIG. 1835: PRO81339

FIG. 1836: DNA324708,NM_{—}002011,gen.NM_{—}002011

FIG. 1837: PRO81340

FIG. 1838: DNA324709,NM_{—}022963,gen.NM_{—}022963

FIG. 1839: PRO81341

FIG. 1840: DNA324710,XM_{—}038946,gen.XM_{—}038946

FIG. 1841: DNA324711,XM_{—}113454,gen.XM_{—}113454

FIG. 1842: DNA324712,XM_{—}166028,gen.XM_{—}166028

FIG. 1843: DNA324713,NM_{—}015043,gen.NM_{—}015043

FIG. 1844: PRO81345

FIG. 1845: DNA324714,XM_{—}113468,gen.XM_{—}113468

FIG. 1846: DNA324715,NM_{—}014275,gen.NM_{—}014275

FIG. 1847: PRO1927

FIG. 1848: DNA324716,NM_{—}054013,gen.NM_{—}054013

FIG. 1849: PRO81347

FIG. 1850: DNA270675,NM_{—}005520,gen.NM_{—}005520

FIG. 1851: PRO59040

FIG. 1852: DNA324717,NM_{—}006098,gen.NM_{—}006098

FIG. 1853: PRO25849

FIG. 1854: DNA269593,NM_{—}005110,gen.NM_{—}005110

FIG. 1855: PRO58006

FIG. 1856: DNA324718,XM_{—}116365,gen.XM_{—}116365

FIG. 1857: DNA324719,XM_{—}116511,gen.XM_{—}116511

FIG. 1858: DNA324720,XM_{—}087823,gen.XM_{—}087823

FIG. 1859A-C: DNA324721,XM_{—}053955,gen.XM_{—}053955

FIG. 1860: DNA324722,XM_{—}113476,gen.XM_{—}113476

FIG. 1861: DNA324723,XM_{—}116514,gen.XM_{—}116514

FIG. 1862: DNA324724,XM_{—}094741,gen.XM_{—}094741

FIG. 1863: DNA324725,NM_{—}025168,gen.NM_{—}025168

FIG. 1864: PRO81354

FIG. 1865A-B: DNA324726,XM_{—}165740,gen.XM_{—}165740

FIG. 1866: DNA272171,NM_{—}002388,gen.NM_{—}002388

FIG. 1867: PRO60438

FIG. 1868: DNA324727,XM_{—}167169,gen.XM_{—}167169

FIG. 1869: PRO81355

FIG. 1870: DNA324728,NM_{—}014452,gen.NM_{—}014452

FIG. 1871: PRO868

FIG. 1872: DNA324729,XM_{—}166349,gen.XM_{—}166349

FIG. 1873: PRO81356

FIG. 1874: DNA304680,NM_{—}007355,gen.NM_{—}007355

FIG. 1875: PRO71106

FIG. 1876: DNA324730,XM_{—}165772,gen.XM_{—}165772

FIG. 1877: DNA324731,XM_{—}168123,gen.XM_{—}168123

FIG. 1878: DNA324732,XM_{—}166457,gen.XM_{—}166457

FIG. 1879: DNA324733,XM_{—}166469,gen.XM_{—}166469

FIG. 1880: DNA324734,NM_{—}018135,gen.NM_{—}018135

FIG. 1881: PRO81359

FIG. 1882A-B: DNA324735,NM_{—}166340,gen.XM_{—}166340

FIG. 1883: DNA324736,XM_{—}087960,gen.XM_{—}087960

FIG. 1884: DNA324737,XM_{—}166362,gen.XM_{—}166362

FIG. 1885: PRO81362

FIG. 1886: DNA227204,NM_{—}015388,gen.NM_{—}015388

FIG. 1887: PRO37667

FIG. 1888: DNA324738,XM_{—}166425 gen.XM_{—}166425

FIG. 1889: PRO81363

FIG. 1890: DNA324739,NM_{—}057161,gen.NM_{—}057161

FIG. 1891: PRO81364

FIG. 1892: DNA270613,NM_{—}006245,gen.NM_{—}006245

FIG. 1893: PRO58984

FIG. 1894: DNA324740,NM_{—}006586,gen.NM_{—}006586

FIG. 1895: PRO81365

FIG. 1896: DNA324741,XM_{—}166402,gen.XM_{—}166402

FIG. 1897: PRO81366

FIG. 1898: DNA324742,NM_{—}001760,gen.NM_{—}001760

FIG. 1899: PRO81367

FIG. 1900: DNA287246,NM_{—}004053,gen.NM_{—}004053

FIG. 1901: PRO69521

FIG. 1902: DNA324743,NM_{—}017601,gen.NM_{—}017601

FIG. 1903: PRO81368

FIG. 1904: DNA275630,NM_{—}006708,gen.NM_{—}006708

FIG. 1905: PRO63253

FIG. 1906: DNA324744,NM_{—}014341,gen.NM_{—}014341

FIG. 1907: PRO81369

FIG. 1908: DNA304460,NM_{—}016059,gen.NM_{—}016059

FIG. 1909: PRO4984

FIG. 1910: DNA324745,XM_{—}166412,gen.XM_{—}166412

FIG. 1911: PRO81370

FIG. 1912: DNA304716,NM_{—}078467,gen.NM_{—}078467

FIG. 1913: PRO71142

FIG. 1914: DNA324746,XM_{—}166417,gen.XM_{—}166417

FIG. 1915: PRO81371

FIG. 1916A-B: DNA324747,NM_{—}003137,gen.NM_{—}003137

FIG. 1917: PRO81372

FIG. 1918A-B: DNA324748,NM_{—}004117,gen.NM_{—}004117

FIG. 1919: PRO36841

FIG. 1920: DNA324749,XM_{—}166419,gen.XM_{—}166419

FIG. 1921: DNA324750,XM_{—}165794,gen.XM_{—}165794

FIG. 1922: DNA324751,NM_{—}007104,gen.NM_{—}007104

FIG. 1923: PRO10360

FIG. 1924: DNA324752,NM_{—}024294,gen.NM_{—}024294

FIG. 1925: PRO81375

FIG. 1926: DNA324753,NM_{—}022758,gen.NM_{—}022758

FIG. 1927: PRO50582

FIG. 1928: DNA324754,XM_{—}168070,gen.XM_{—}168070

FIG. 1929: DNA324755,NM_{—}012391,gen.NM_{—}012391

FIG. 1930: PRO81377

FIG. 1931: DNA324756,XM_{—}166459,gen.XM_{—}166459

FIG. 1932: DNA324757,XM_{—}166333,gen.XM_{—}166333

FIG. 1933: PRO81379

FIG. 1934: DNA324758,XM_{—}058039,gen.XM_{—}058039

FIG. 1935: PRO81380

FIG. 1936: DNA324759,XM_{—}087990,gen.XM_{—}087990

FIG. 1937: DNA324760,XM_{—}165743,gen.XM_{—}165743

FIG. 1938: DNA324761,XM_{—}166360,gen.XM_{—}166360

FIG. 1939: DNA324763,XM_{—}059801,gen.XM_{—}059801

FIG. 1940: DNA324764,XM_{—}166363,gen.XM_{—}166363

FIG. 1941: DNA324765,XM_{—}016857,gen.XM_{—}016857

FIG. 1942: DNA227442,NM_{—}001350,gen.NM_{—}001350

FIG. 1943: PRO37905

FIG. 1944: DNA324766,NM_{—}005452,gen.NM_{—}005452

FIG. 1945: PRO81387

FIG. 1946: DNA30466 1,NM_{—}022551,gen.NM_{—}022551

FIG. 1947: PRO71088

FIG. 1948: DNA324767,XM_{—}165747,gen.XM_{—}165747

FIG. 1949: DNA324768,XM_{—}165698,gen.XM_{—}165698

FIG. 1950: PRO4884

FIG. 1951A-B: DNA324769,XM_{—}165770,gen.XM_{—}165770

FIG. 1952: DNA287227,NM_{—}004159,gen.NM_{—}004159

FIG. 1953: PRO69506

FIG. 1954: DNA324770,XM_{—}165717,gen.XM_{—}165717

FIG. 1955: DNA324771,XM_{—}166480,gen.XM_{—}166480

FIG. 1956: DNA324772,XM_{—}165801,gen.XM_{—}165801

FIG. 1957A-B: DNA324773,NM_{—}000592,gen.NM_{—}000592

FIG. 1958: PRO36316

FIG. 1959: DNA324774,NM_{—}001710,gen.NM_{—}001710

FIG. 1960: PRO36305

FIG. 1961: DNA227607,NM_{—}005346,gen.NM_{—}005346

FIG. 1962: PRO38070

FIG. 1963: DNA304668,NM_{—}005345,gen.NM_{—}005345

FIG. 1964: PRO71095

FIG. 1965: DNA324775,NM_{—}021177,gen.NM_{—}021177

FIG. 1966: PRO81394

FIG. 1967A-B: DNA272263,NM_{—}006295,gen.NM_{—}006295

FIG. 1968: PRO70138

FIG. 1969: DNA287319,NM_{—}001288,gen.NM_{—}001288

FIG. 1970: PRO69584

FIG. 1971: DNA324776,NM_{—}001320,gen.NM_{—}001320

FIG. 1972: PRO63052

FIG. 1973A-B: DNA324777,NM_{—}004639,gen.NM_{—}004639

FIG. 1974: PRO81395

FIG. 1975A-B: DNA324778,NM_{—}080703,gen.NM_{—}080703

FIG. 1976: PRO81396

FIG. 1977A-B: DNA324779,NM_{—}080702,gen.NM_{—}080702

FIG. 1978: PRO81397

FIG. 1979A-B: DNA324780,NM_{—}004638,gen.NM_{—}004638

FIG. 1980: PRO81398

FIG. 1981A-B: DNA324781,NM_{—}080686,gen.NM_{—}080686

FIG. 1982: PRO81399

FIG. 1983: DNA324782,XM_{—}165771,gen.XM_{—}165771

FIG. 1984: DNA324783,NM_{—}080598,gen.NM_{—}080598

FIG. 1985: PRO71125

FIG. 1986: DNA304699,NM_{—}004640,gen.NM_{—}004640

FIG. 1987: PRO71125

FIG. 1988: DNA324784,XM_{—}165765,gen.XM_{—}165765

FIG. 1989: PRO81400

FIG. 1990: DNA324785,XM_{—}087945,gen.XM_{—}087945

FIG. 1991: PRO81401

FIG. 1992: DNA324786,XM_{—}166381,gen.XM_{—}166381

FIG. 1993: PRO81402

FIG. 1994: DNA324787,XM_{—}168104,gen.XM_{—}168104

FIG. 1995: DNA324788,XM_{—}166401,gen.XM_{—}166401

FIG. 1996: PRO81404

FIG. 1997: DNA271040,NM_{—}001517,gen.NM_{—}001517

FIG. 1998: PRO59365

FIG. 1999A-B: DNA324789,XM_{—}165738,gen.XM_{—}165738

FIG. 2000: DNA324790,XM_{—}087939,gen.XM_{—}087939

FIG. 2001: PRO81406

FIG. 2002: DNA324791,XM_{—}166353,gen.XM_{—}166353

FIG. 2003: PRO1112

FIG. 2004A-B: DNA324792,XM_{—}166376,gen.XM_{—}166376

FIG. 2005: PRO81407

FIG. 2006A-B: DNA324793,XM_{—}165799,gen.XM_{—}165799

FIG. 2007: DNA290264,NM_{—}025263,gen.NM_{—}025263

FIG. 2008: PRO70393

FIG. 2009: DNA324794,XM_{—}166361,gen.XM_{—}166361

FIG. 2010: PRO81409

FIG. 2011: DNA324795,XM_{—}165764,gen.XM_{—}165764

FIG. 2012: PRO81410

FIG. 2013: DNA324796,XM_{—}165758,gen.XM_{—}165758

FIG. 2014: PRO81411

FIG. 2015: DNA324797,XM_{—}166406,gen.XM_{—}166406

FIG. 2016: DNA324798,XM_{—}165809,gen.XM_{—}165809

FIG. 2017: DNA324799,NM_{—}018950,gen.NM_{—}018950

FIG. 2018: PRO81414

FIG. 2019: DNA324800,XM_{—}166392,gen.XM_{—}166392

FIG. 2020: PRO81415

FIG. 2021: DNA324801,XM_{—}166336,gen.XM_{—}166336

FIG. 2022: PRO81416

FIG. 2023: DNA324802,XM_{—}1 67128,gen.XM_{—}167128

FIG. 2024: PRO23797

FIG. 2025: DNA324803,XM_{—}167161,gen.XM_{—}2167161

FIG. 2026: PRO81417

FIG. 2027: DNA324804,NM_{—}013375,gen.NM_{—}013375

FIG. 2028: PRO81418

FIG. 2029: DNA324805,NM_{—}007047,gen.NM_{—}007047

FIG. 2030: PRO81419

FIG. 2031: DNA324806,XM_{—}167179,gen.XM_{—}167179

FIG. 2032: DNA290785,NM_{—}003107,gen.NM_{—}003107

FIG. 2033: PRO70544

FIG. 2034: DNA150772,NM_{—}003472,gen.NM_{—}003472

FIG. 2035: PRO12797

FIG. 2036A-B: DNA324807,XM_{—}165728,gen.NM_{—}165728

FIG. 2037: DNA324808,XM_{—}165749,gen.XM_{—}165749

FIG. 2038: PRO81421

FIG. 2039A-B: DNA324809,NM_{—}004973,gen.NM_{—}004973

FIG. 2040: PRO81422

FIG. 2041: DNA324810,XM_{—}167196,gen.XM_{—}167196

FIG. 2042: DNA324811,XM_{—}166446,gen.XM_{—}166446

FIG. 2043: PRO81424

FIG. 2044A-C: DNA324812,XM_{—}165777,gen.XM_{—}165777

FIG. 2045: DNA324813,XM_{—}037875,gen.XM_{—}037875

FIG. 2046: PRO81426

FIG. 2047: DNA324814,XM_{—}167225,gen.XM_{—}167225

FIG. 2048: PRO81427

FIG. 2049: DNA324815,XM_{—}166357,gen.XM_{—}166357

FIG. 2050: DNA324816,NM_{—}001069,gen.NM_{—}001069

FIG. 2051: PRO81429

FIG. 2052: DNA324817,NM_{—}001500,gen.NM_{—}001500

FIG. 2053: PRO81430

FIG. 2054A-B: DNA324818,XM_{—}166042,gen.XM_{—}166042

FIG. 2055: PRO51389

FIG. 2056: DNA324819,XM_{—}052721,gen.XM_{—}052721

FIG. 2057: DNA324820,XM_{—}165499,gen.XM_{—}165499

FIG. 2058: DNA324821,XM_{—}114497,gen.XM_{—}114497

FIG. 2059: DNA324822,XM_{—}011117,gen.XM_{—}011117

FIG. 2060: DNA324823,XM_{—}094855,gen.XM_{—}094855

FIG. 2061: PRO81435

FIG. 2062: DNA324824,XM_{—}059776,gen.XM_{—}059776

FIG. 2063: PRO81436

FIG. 2064: DNA324825,XM_{—}055641,gen.XM_{—}055641

FIG. 2065: DNA324826,XM_{—}004151,gen.XM_{—}004151

FIG. 2066: DNA324827,NM_{—}133645,gen.NM_{—}133645

FIG. 2067: PRO81439

FIG. 2068: DNA324828,XM_{—}097453,gen.XM_{—}097453

FIG. 2069: DNA324829,XM_{—}029228,gen.XM_{—}029228

FIG. 2070: DNA103471,NM_{—}006670,gen.NM_{—}006670

FIG. 2071: PRO4798

FIG. 2072: DNA324830,XM_{—}068963,gen.XM_{—}68963

FIG. 2073: PRO81441

FIG. 2074: DNA324831,XM_{—}040623,gen.XM_{—}040623

FIG. 2075: DNA324832,NM_{—}020320,gen.NM_{—}020320

FIG. 2076: PRO81443

FIG. 2077: DNA324833,NM_{—}014107,gen.NM_{—}014107

FIG. 2078: PRO81444

FIG. 2079A-B: DNA324834,XM_{—}084204,gen.XM_{—}84204

FIG. 2080: DNA324835,XM_{—}017517,gen.XM_{—}17517

FIG. 2081: DNA324836,NM_{—}032929,gen.NM_{—}32929

FIG. 2082: PRO81446

FIG. 2083: DNA324837,XM_{—}003611,gen.XM_{—}03611

FIG. 2084: PRO81447

FIG. 2085: DNA324838,XM_{—}068919,gen.XM_{—}068919

FIG. 2086: PRO81448

FIG. 2087: DNA324839,XM_{—}167016,gen.XM_{—}167016

FIG. 2088: PRO81449

FIG. 2089: DNA324840,XM_{—}087855,gen.XM_{—}087855

FIG. 2090: DNA324841,XM_{—}087853,gen.XM_{—}087853

FIG. 2091: DNA324842,XM_{—}165669,gen.XM_{—}165669

FIG. 2092: DNA324843,XM_{—}166303,gen.XM_{—}166303

FIG. 2093: PRO81453

FIG. 2094: DNA324844,XM_{—}167027,gen.XM_{—}167027

FIG. 2095: PRO81454

FIG. 2096: DNA324845,XM_{—}167037,gen.XM_{—}167037

FIG. 2097: PRO81455

FIG. 2098: DNA324846,XM_{—}018182,gen.XM_{—}018182

FIG. 2099: DNA227924,NM_{—}000165,gen.NM_{—}000165

FIG. 2100: PRO38387

FIG. 2101: DNA324847,XM_{—}166310,gen.XM_{—}166310

FIG. 2102: PRO81457

FIG. 2103: DNA324848,XM_{—}168054,gen.XM_{—}168054

FIG. 2104: DNA271418,NM_{—}003287,gen.NM_{—}003287

FIG. 2105: PRO59717

FIG. 2106: DNA324849,XM_{—}114492,gen.XM_{—}114492

FIG. 2107: DNA324850,XM_{—}037056,gen.XM_{—}037056

FIG. 2108: DNA32485 1,XM_{—}098468,gen.XM_{—}098468

FIG. 2109: PRO19933

FIG. 2110: DNA324852,XM_{—}004526,gen.XM_{—}004526

FIG. 2111: DNA324853,NM_{—}001016,gen.NM_{—}001016

FIG. 2112: PRO81462

FIG. 2113: DNA324854,XM_{—}004297,gen.XM_{—}004297

FIG. 2114: DNA324855,XM_{—}004256,gen.XM_{—}004256

FIG. 2115: PRO81464

FIG. 2116: DNA324856,NM_{—}014320,gen.NM_{—}014320

FIG. 2117: PRO81465

FIG. 2118: DNA324857,XM_{—}059741,gen.XM_{—}059741

FIG. 2119: DNA324858,XM_{—}017831,gen.XM_{—}017831

FIG. 2120: PRO81467

FIG. 2121: DNA324859,XM_{—}049899,gen.XM_{—}049899

FIG. 2122: DNA324860,XM_{—}004379,gen.XM_{—}004379

FIG. 2123A-C: DNA324861,XM_{—}087834,gen.XM_{—}087834

FIG. 2124A-B: DNA324862,XM_{—}087836,gen.XM_{—}087836

FIG. 2125: PRO81471

FIG. 2126: DNA324863,NM_{—}005389,gen.NM_{—}005389

FIG. 2127: PRO66279

FIG. 2128A-C: DNA324864,XM_{—}029746,gen.XM_{—}029746

FIG. 2129: PRO66282

FIG. 2130: DNA324865,XM_{—}004383,gen.XM_{—}004383

FIG. 2131: DNA324866,XM_{—}059745,gen.XM_{—}059745

FIG. 2132: DNA324867,XM_{—}033912,gen.XM_{—}033912

FIG. 2133: PRO81474

FIG. 2134: DNA324868,XM_{—}033910,gen.XM_{—}033910

FIG. 2135: DNA324870,NM_{—}003181,gen.NM_{—}003181

FIG. 2136: PRO81476

FIG. 2137: DNA324871,NM_{—}002793,gen.NM_{—}002793

FIG. 2138: PRO81477

FIG. 2139: DNA324872,XM_{—}044866,gen.XM_{—}044866

FIG. 2140: DNA324873,XM_{—}116524,gen.XM_{—}116524

FIG. 2141: DNA324874,XM_{—}059773,gen.XM_{—}059773

FIG. 2142: DNA324875,XM_{—}084998,gen.XM_{—}084998

FIG. 2143: PRO81481

FIG. 2144: DNA324876,XM_{—}058266,gen.XM_{—}058266

FIG. 2145: DNA324877,XM_{—}042422,gen.XM_{—}042422

FIG. 2146A-B: DNA324878,XM_{—}054706,gen.XM_{—}054706

FIG. 2147: DNA324879,XM_{—}166049,gen.XM_{—}166049

FIG. 2148: DNA324880,XM_{—}042473,gen.XM_{—}042473

FIG. 2149: PRO81486

FIG. 2150: DNA324881,XM_{—}167046,gen.XM_{—}167046

FIG. 2151: PRO23797

FIG. 2152: DNA324882,XM_{—}071937,gen.XM_{—}071937

FIG. 2153: PRO81487

FIG. 2154: DNA324883,XM_{—}087991,gen.XM_{—}087991

FIG. 2155: DNA324884,NM_{—}005514,gen.NM_{—}005514

FIG. 2156: PRO81490

FIG. 2157: DNA324885,XM_{—}166327,gen.XM_{—}166327

FIG. 2158: PRO81491

FIG. 2159: DNA324886,XM_{—}165692,gen.XM_{—}165692

FIG. 2160: DNA324887,XM_{—}117449,gen.XM_{—}117449

FIG. 2161: DNA324888,XM_{—}086428,gen.XM_{—}086428

FIG. 2162: PRO81494

FIG. 2163: DNA324889,NM_{—}032350,gen.NM_{—}032350

FIG. 2164: PRO81495

FIG. 2165: DNA324890,NM_{—}013393,gen.NM_{—}013393

FIG. 2166: PRO81496

FIG. 2167: DNA324891,XM_{—}165860,gen.XM_{—}165860

FIG. 2168: DNA324892,XM_{—}166541,gen.XM_{—}166541

FIG. 2169: PRO81498

FIG. 2170A-B: DNA324893,XM_{—}166523,gen.XM_{—}166523

FIG. 2171: PRO81499

FIG. 2172: DNA324894,NM_{—}016003,gen.NM_{—}016003

FIG. 2173: PRO81500

FIG. 2174: DNA225631,NM_{—}001101,gen.NM_{—}001101

FIG. 2175: PRO36094

FIG. 2176: DNA274326,NM_{—}003088,gen.NM_{—}003088

FIG. 2177: PRO62244

FIG. 2178: DNA324895,NM_{—}006303,gen.NM_{—}006303

FIG. 2179: PRO81501

FIG. 2180: DNA324896,NM_{—}014413,gen.NM_{—}014413

FIG. 2181: PRO60579

FIG. 2182: DNA247595,NM_{—}006908,gen.NM_{—}006908

FIG. 2183: PRO45014

FIG. 2184: DNA324897,NM_{—}006854,gen.NM_{—}006854

FIG. 2185: PRO12468

FIG. 2186: DNA324898,NM_{—}024067,gen.NM_{—}024067

FIG. 2187: PRO81502

FIG. 2188: DNA324899,NM_{—}002947,gen.NM_{—}002947

FIG. 2189: PRO81503

FIG. 2190: DNA324900,XM_{—}166531,gen.XM_{—}166531

FIG. 2191: DNA324901,XM_{—}166540,gen.XM_{—}166540

FIG. 2192: PRO81505

FIG. 2193: DNA193955,NM_{—}002489,gen.NM_{—}002489

FIG. 2194: PRO23362

FIG. 2195: DNA324902,XM_{—}088264,gen.XM_{—}088264

FIG. 2196: PRO81506

FIG. 2197: DNA324903,XM_{—}165841,gen.XM_{—}165841

FIG. 2198: DNA324904,XM_{—}166521,gen.XM_{—}166521

FIG. 2199: PRO81508

FIG. 2200: DNA324905,XM_{—}166506,gen.XM_{—}166506

FIG. 2201: PRO81509

FIG. 2202: DNA324906,XM_{—}166505,gen.XM_{—}166505

FIG. 2203: DNA324907,XM_{—}166514,gen.XM_{—}166514

FIG. 2204: DNA324908,XM_{—}166515,gen.XM_{—}166515

FIG. 2205: DNA324909,XM_{—}166512,gen.XM_{—}166512

FIG. 2206: DNA227929,NM_{—}019059,gen.NM_{—}019059

FIG. 2207: PRO38392

FIG. 2208A-B: DNA324910,NM_{—}18947,gen.NM_{—}018947

FIG. 2209: PRO81514

FIG. 2210: DNA324911,NM_{—}002137,gen.NM_{—}002137

FIG. 2211: PRO81515

FIG. 2212: DNA324912,NM_{—}031243,gen.NM_{—}031243

FIG. 2213: PRO6373

FIG. 2214: DNA324913,NM_{—}007276,gen.NM_{—}007276

FIG. 2215: PRO81516

FIG. 2216: DNA324914,NM_{—}016587,gen.NM_{—}016587

FIG. 2217: PRO81517

FIG. 2218: DNA324915,XM_{—}040853,gen.XM_{—}040853

FIG. 2219: DNA324916,XM_{—}166509,gen.XM_{—}166509

FIG. 2220: DNA324917,XM_{—}166513,gen.XM_{—}166513

FIG. 2221: PRO81520

FIG. 2222: DNA324918,XM_{—}166504,gen.XM_{—}166504

FIG. 2223: PRO81521

FIG. 2224: DNA324919,XM_{—}166494,gen.XM_{—}166494

FIG. 2225: DNA324920,XM_{—}107825,gen.XM_{—}107825

FIG. 2226A-B: DNA324921,NM_{—}022748,gen.NM_{—}022748

FIG. 2227: PRO81523

FIG. 2228: DNA324922,NM_{—}000598,gen.NM_{—}000598

FIG. 2229: PRO119

FIG. 2230A-B: DNA324923,XM_{—}166594,gen.XM_{—}166594

FIG. 2231: PRO81524

FIG. 2232A-B: DNA275334,NM_{—}030900,gen.NM_{—}030900

FIG. 2233: PRO63009

FIG. 2234: DNA324924,NM_{—}031443,gen.NM_{—}031443

FIG. 2235: PRO81525

FIG. 2236: DNA324925,NM_{—}012412,gen.NM_{—}012412

FIG. 2237: PRO61812

FIG. 2238: DNA324926,NM_{—}021130,gen.NM_{—}021130

FIG. 2239: PRO7427

FIG. 2240A-B: DNA324927,XM_{—}165877,gen.XM_{—}165877

FIG. 2241: PRO81526

FIG. 2242: DNA227268,NM_{—}019082,gen.NM_{—}019082

FIG. 2243: PRO37731

FIG. 2244: DNA324928,XM_{—}015258,gen.XM_{—}015258

FIG. 2245: DNA324929,XM_{—}165870,gen.XM_{—}165870

FIG. 2246: DNA273865,NM_{—}006230,gen.NM_{—}006230

FIG. 2247: PRO61824

FIG. 2248A-B: DNA324930,XM_{—}165882,gen.XM_{—}165882

FIG. 2249: DNA324931,XM_{—}165867,gen.XM_{—}165867

FIG. 2250: PRO61688

FIG. 2251: DNA324932,NM_{—}014063,gen.NM_{—}014063

FIG. 2252: PRO81529

FIG. 2253: DNA324933,XM_{—}165872,gen.XM_{—}165872

FIG. 2254: DNA304707,NM_{—}002787,gen.NM_{—}002787

FIG. 2255: PRO71133

FIG. 2256: DNA324934,XM_{—}016733,gen.XM_{—}016733

FIG. 2257: PRO81531

FIG. 2258: DNA324935,XM_{—}165876,gen.XM_{—}165876

FIG. 2259A-B: DNA324936,NM_{—}014800,gen.NM_{—}014800

FIG. 2260: DNA324937,NM_{—}130442,gen.NM_{—}130442

FIG. 2261: PRO81534

FIG. 2262: DNA226416,NM_{—}000385,gen.NM_{—}000385

FIG. 2263: PRO36879

FIG. 2264A-B: DNA324938,XM_{—}167339,gen.XM_{—}167339

FIG. 2265: DNA287189,NM_{—}002047,gen.NM_{—}002047

FIG. 2266: PRO69475

FIG. 2267: DNA324939,XM_{—}170195,gen.XM_{—}170195

FIG. 2268: PRO81536

FIG. 2269: DNA324940,XM_{—}168378,gen.XM_{—}168378

FIG. 2270: PRO81537

FIG. 2271: DNA324941,XM_{—}168354,gen.XM_{—}168354

FIG. 2272: PRO81538

FIG. 2273: DNA324942,XM_{—}167494,gen.XM_{—}167494

FIG. 2274: DNA103588,NM_{—}001762,gen.NM_{—}001762

FIG. 2275: PRO4912

FIG. 2276: DNA324943,XM_{—}037741,gen.XM_{—}037741

FIG. 2277: PRO81540

FIG. 2278: DNA324944,XM_{—}050265,gen.XM_{—}050265

FIG. 2279: PRO81541

FIG. 2280: DNA324945,XM_{—}017483,gen.XM_{—}017483

FIG. 2281A-B: DNA324946,XM_{—}018359,gen.XM_{—}018359

FIG. 2282: DNA324947,XM_{—}059876,gen.XM_{—}059876

FIG. 2283: PRO81544

FIG. 2284: DNA324948,NM_{—}032951,gen.NM_{—}032951

FIG. 2285: PRO81545

FIG. 2286: DNA324949,NM_{—}032953,gen.NM_{—}032953

FIG. 2287: PRO81546

FIG. 2288: DNA324950,NM_{—}022170,gen.NM_{—}022170

FIG. 2289: PRO81547

FIG. 2290: DNA324951,NM_{—}031992,gen.NM_{—}031992

FIG. 2291: PRO81548

FIG. 2292: DNA324952,XM_{—}004901,gen.XM_{—}004901

FIG. 2293: DNA324953,NM_{—}016328,gen.NM_{—}016328

FIG. 2294: PRO81550

FIG. 2295A-B: DNA324954,NM_{—}032999,gen.NM_{—}032999

FIG. 2296: PRO81551

FIG. 2297: DNA324955,XM_{—}088239,gen.XM_{—}088239

FIG. 2298: PRO81552

FIG. 2299A-B: DNA324956,XM_{—}167500,gen.XM_{—}167500

FIG. 2300A-B: DNA324957,XM_{—}167504,gen.XM_{—}167504

FIG. 2301: DNA324958,XM_{—}167498,gen.XM_{—}167498

FIG. 2302: DNA324959,XM_{—}168454,gen.XM_{—}168454

FIG. 2303: PRO81556

FIG. 2304: DNA324960,NM_{—}031925,gen.NM_{—}031925

FIG. 2305: PRO81557

FIG. 2306: DNA324961,NM_{—}005918,gen.NM_{—}005918

FIG. 2307: PRO81558

FIG. 2308: DNA304710,NM_{—}001540,gen.NM_{—}001540

FIG. 2309: PRO71136

FIG. 2310: DNA324962,XM_{—}168470,gen.XM_{—}168470

FIG. 2311: DNA324963,XM_{—}168461,gen.XM_{—}168461

FIG. 2312A-B: DNA324964,XM_{—}167502,gen.XM_{—}167502

FIG. 2313: DN74 24965,XM_{—}17442,gen.XM_{—}017442

FIG. 2314: PRO81561

FIG. 2315: DNA324966,XM_{—}168450,gen.XM_{—}168450

FIG. 2316: DNA324967,XM_{—}168435,gen.XM_{—}168435

FIG. 2317: DNA324968,XM_{—}168464,gen.XM_{—}168464

FIG. 2318: DNA324969,XM_{—}170427,gen.XM_{—}170427

FIG. 2319A-B: DNA324971,NM_{—}015068,gen.NM_{—}015068

FIG. 2320: PRO81566

FIG. 2321A-B: DNA324972,XM_{—}167476,gen.XM_{—}167476

FIG. 2322: DNA324973,XM_{—}168181,gen.XM_{—}168181

FIG. 2323: DNA324974,XM_{—}168251,gen.XM_{—}168251

FIG. 2324: PRO81569

FIG. 2325: DNA324975,XM_{—}167477,gen.XM_{—}167477

FIG. 2326: DNA324976,NM_{—}005837,gen.NM_{—}005837

FIG. 2327: PRO81571

FIG. 2328: DNA324977,XM_{—}167483,gen.XM_{—}167483

FIG. 2329: DNA324978,XM_{—}167484,gen.XM_{—}167484

FIG. 2330: PRO81572

FIG. 2331: DNA324979,NM_{—}030935,gen.NM_{—}030935

FIG. 2332: PRO81573

FIG. 2333: DNA324980,NM_{—}019606,gen.NM_{—}019606

FIG. 2334: PRO81574

FIG. 2335: DNA324981,NM_{—}024070,gen.NM_{—}024070

FIG. 2336: PRO81575

FIG. 2337: DNA324982,XM_{—}084241,gen.XM_{—}084241

FIG. 2338: DNA324983,NM_{—}006833,gen.NM_{—}006833

FIG. 2339: PRO22897

FIG. 2340: DNA324984,NM_{—}032164,gen.NM_{—}032164

FIG. 2341: PRO81578

FIG. 2342: DNA304801,NM_{—}004889,gen.NM_{—}004889

FIG. 2343: PRO71211

FIG. 2344: DNA324985,NM_{—}006693,gen.NM_{—}006693

FIG. 2345: PRO81579

FIG. 2346: DNA324986,XM_{—}165839,gen.XM_{—}165839

FIG. 2347: PRO81580

FIG. 2348: DNA272090,NM_{—}005720,gen.NM_{—}005720

FIG. 2349: PRO60360

FIG. 2350: DNA324987,XM_{—}165836,gen.XM_{—}165836

FIG. 2351A-B: DNA324988,XM_{—}166482,gen.XM_{—}166482

FIG. 2352: DNA324989,XM_{—}088180,gen.XM_{—}088180

FIG. 2353A-B: DNA324990,XM_{—}166485,gen.XM_{—}166485

FIG. 2354: PRO81584

FIG. 2355: DNA324991,NM_{—}001673,gen.NM_{—}001673

FIG. 2356: PRO81585

FIG. 2357: DNA324992,NM_{—}133436,gen.NM_{—}133436

FIG. 2358: PRO81586

FIG. 2359: DNA324993,XM_{—}168586,gen.XM_{—}168586

FIG. 2360: PRO81587

FIG. 2361: DNA83141,NM_{—}000602,gen.NM_{—}000602

FIG. 2362: PRO2604

FIG. 2363: DNA324994,NM_{—}057089,gen.NM_{—}057089

FIG. 2364: PRO81588

FIG. 2365: DNA324995,NM_{—}001283,gen.NM_{—}001283

FIG. 2366: PRO41882

FIG. 2367: DNA324996,NM_{—}003378,gen.NM_{—}003378

FIG. 2368: PRO81589

FIG. 2369: DNA324997,NM_{—}001084,gen.NM_{—}001084

FIG. 2370: PRO58437

FIG. 2371: DNA270711,NM_{—}006349,gen.NM_{—}006349

FIG. 2372: PRO59074

FIG. 2373: DNA324998,NM_{—}024653,gen.NM_{—}024653

FIG. 2374: PRO81590

FIG. 2375: DNA824999,XM_{—}168548,gen.XM_{—}168548

FIG. 2376: DNA325000,NM_{—}032958,gen.NM_{—}032958

FIG. 2377: PRO81591

FIG. 2378: DNA325001,NM_{—}002803,gen.NM_{—}002803

FIG. 2379: PRO81592

FIG. 2380: DNA325002,XM_{—}168572,gen.XM_{—}168572

FIG. 2381: DNA325003,XM_{—}071605,gen.XM_{—}071605

FIG. 2382: PRO81594

FIG. 2383: DNA325004,XM_{—}033876,gen.XM_{—}033876

FIG. 2384: PRO81595

FIG. 2385A-B: DNA325005,XM_{—}027214,gen.XM_{—}027214

FIG. 2386: DNA325006,XM_{—}088073,gen.XM_{—}088073

FIG. 2387: DNA325007,XM_{—}072430,gen.XM_{—}072430

FIG. 2388: PRO81598

FIG. 2389: DNA325008,XM_{—}050430,gen.XM_{—}050430

FIG. 2390: PRO81599

FIG. 2391: DNA325009,NM_{—}001753,gen.NM_{—}001753

FIG. 2392: PRO81600

FIG. 2393: DNA226560,NM_{—}006136,gen.NM_{—}006136

FIG. 2394: PRO37023

FIG. 2395: DNA325010,XM_{—}012284,gen.XM_{—}012284

FIG. 2396: DNA325011,NM_{—}005000,gen.NM_{—}005000

FIG. 2397: PRO59380

FIG. 2398: DNA325012,NM_{—}001662,gen.NM_{—}001662

FIG. 2399: PRO39773

FIG. 2400: DNA325013,XM_{—}011618,gen.XM_{—}011618

FIG. 2401: PRO81602

FIG. 2402: DNA325014,XM_{—}004627,gen.XM_{—}004627

FIG. 2403: DNA325015,XM_{—}045401,gen.XM_{—}045401

FIG. 2404: DNA325016,XM_{—}114602,gen.XM_{—}4114602

FIG. 2405: PRO81605

FIG. 2406: DNA325017,XM_{—}117481,gen.XM_{—}117481

FIG. 2407A-C: DNA325018,XM_{—}045856,gen.XM_{—}045856

FIG. 2408: PRO81607

FIG. 2409A-B: DNA325019,XM_{—}088105,gen.XM_{—}088105

FIG. 2410: PRO81608

FIG. 2411: DNA325020,XM_{—}011548,gen.XM_{—}011548

FIG. 2412: PRO81609

FIG. 2413: DNA325021,XM_{—}045952,gen.XM_{—}045952

FIG. 2414: DNA325022,XM_{—}046001,gen.XM_{—}046001

FIG. 2415: PRO81611

FIG. 2416: DNA325023,XM_{—}088099,gen.XM_{—}088099

FIG. 2417: DNA325024,XM_{—}040498,gen.XM_{—}040498

FIG. 2418: DNA325025,XM_{—}088103,gen.XM_{—}088103

FIG. 2419: PRO81614

FIG. 2420: DNA325026,XM_{—}088122,gen.XM_{—}088122

FIG. 2421: PRO81615

FIG. 2422: DNA325027,XM_{—}088119,gen.XM_{—}088119

FIG. 2423: DNA325028,NM_{—}001628,gen.NM_{—}001628

FIG. 2424: PRO81617

FIG. 2425: DNA325029,NM_{—}020299,gen.NM_{—}020299

FIG. 2426: PRO81618

FIG. 2427: DNA325030,NM_{—}024033,gen.NM_{—}024033

FIG. 2428: PRO81619

FIG. 2429: DNA325031,XM_{—}114555,gen.XM_{—}114555

FIG. 2430: DNA325032,XM_{—}059839,gen.XM_{—}059839

FIG. 2431: PRO81621

FIG. 2432: DNA325033,XM_{—}095146,gen.XM_{—}095146

FIG. 2433: DNA325034,XM_{—}016700,gen.XM_{—}016700

FIG. 2434: DNA325035,XM_{—}042781,gen.XM_{—}042781

FIG. 2435: DNA304685,NM_{—}003143,gen.NM_{—}003143

FIG. 2436: PRO71111

FIG. 2437: DNA325036,NM_{—}018238,gen.NM_{—}018238

FIG. 2438: PRO81625

FIG. 2439: DNA325037,XM_{—}035107,gen.XM_{—}035107

FIG. 2440: DNA325038,NM_{—}003461,gen.NM_{—}003461

FIG. 2441: PRO10194

FIG. 2442: DNA325039,NM_{—}004911,gen.NM_{—}004911

FIG. 2443: PRO2733

FIG. 2444A-B: DNA325040,XM_{—}114578,gen.XM_{—}114578

FIG. 2445: PRO81627

FIG. 2446: DNA325041,XM_{—}088135,gen.XM_{—}088135

FIG. 2447: DNA325042,XM_{—}098654,gen.XM_{—}098654

FIG. 2448: PRO81629

FIG. 2449: DNA325043,NM_{—}023942,gen.NM_{—}023942

FIG. 2450: PRO81630

FIG. 2451: DNA325044,NM_{—}138434,gen.NM_{—}138434

FIG. 2452: PRO81631

FIG. 2453: DNA325045,XM_{—}084238,gen.XM_{—}084238

FIG. 2454A-B: DNA325046,XM_{—}032216,gen.XM_{—}032216

FIG. 2455A-B: DNA325047,XM_{—}032121,gen.XM_{—}032121

FIG. 2456: DNA325048,NM_{—}031434,gen.NM_{—}031434

FIG. 2457: PRO1555

FIG. 2458: DNA226337,NM_{—}005692,gen.NM_{—}005692

FIG. 2459: PRO36800

FIG. 2460: DNA325049,NM_{—}005614,gen.NM_{—}005614

FIG. 2461: PRO37938

FIG. 2462A-B: DNA325050,NM_{—}053043,gen.NM_{—}053043

FIG. 2463: PRO81634

FIG. 2464: DNA325051,NM_{—}022458,gen.NM_{—}022458

FIG. 2465: PRO81635

FIG. 2466: DNA325052,XM_{—}098669,gen.XM_{—}098669

FIG. 2467: DNA325053,NM_{—}017760,gen.NM_{—}017760

FIG. 2468: PRO81637

FIG. 2469: DNA325054,XM_{—}036413,gen.XM_{—}036413

FIG. 2470A-B: DNA325055,XM_{—}032944,gen.XM_{—}032944

FIG. 2471: DNA325056,XM_{—}117444,gen.XM_{—}117444

FIG. 2472: DNA325057,XM_{—}117452,gen.XM_{—}117452

FIG. 2473: DNA325058,XM_{—}070203,gen.XM_{—}070203

FIG. 2474: PRO81641

FIG. 2475: DNA325059,XM_{—}095371,gen.XM_{—}095371

FIG. 2476: DNA325060,NM_{—}004084,gen.NM_{—}004084

FIG. 2477: PRO2570

FIG. 2478: DNA325061,NM_{—}005217,gen.NM_{—}005217

FIG. 2479: PRO9980

FIG. 2480: DNA325062,XM_{—}070188,gen.XM_{—}070188

FIG. 2481: PRO81643

FIG. 2482: DNA325063,XM_{—}035680,gen.XM_{—}035680

FIG. 2483: DNA325064,XM_{—}035662,gen.XM_{—}035662

FIG. 2484: PRO3344

FIG. 2485: DNA325065,XM_{—}005305,gen.XM_{—}005305

FIG. 2486: PRO81645

FIG. 2487: DNA325066,XM_{—}050293,gen.XM_{—}050293

FIG. 2488A-B: DNA325067,XM_{—}027679,gen.XM_{—}027679

FIG. 2489: PRO81647

FIG. 2490A-B: DNA325068,XM_{—}027651,gen.XM_{—}027651

FIG. 2491: DNA274178,NM_{—}005775,gen.NM_{—}005775

FIG. 2492: PRO62108

FIG. 2493: DNA325069,XM_{—}113557,gen.XM_{—}113557

FIG. 2494: PRO81649

FIG. 2495: DNA83022,NM_{—}001199,gen.NM_{—}001199

FIG. 2496: PRO2042

FIG. 2497: DNA325070,NM_{—}006128,gen.NM_{—}006128

FIG. 2498: PRO81650

FIG. 2499: DNA325071,NM_{—}006131,gen.NM_{—}006131

FIG. 2500: PRO81651

FIG. 2501: DNA325072,NM_{—}006132,gen.NM_{—}006132

FIG. 2502: PROS1652

FIG. 2503: DNA325073,NM_{—}025232,gen.NM_{—}025232

FIG. 2504: PRO81653

FIG. 2505: DNA325074,XM_{—}027440,gen.XM_{—}027440

FIG. 2506: DNA225671,NM_{—}001831,gen.NM_{—}001831

FIG. 2507: PRO36134

FIG. 2508: DNA325075,NM_{—}024567,gen.NM_{—}024567

FIG. 2509: PRO81654

FIG. 2510: DNA325076,NM_{—}018250,gen.NM_{—}018250

FIG. 2511: PRO81655

FIG. 2512: DNA227267,NM_{—}018660,gen.NM_{—}018660

FIG. 2513: PRO37730

FIG. 2514A-B: DNA325077,XM_{—}095545,gen.XM_{—}095545

FIG. 2515: DNA325078,XM_{—}088338,gen.XM_{—}088338

FIG. 2516: PRO81657

FIG. 2517: DNA325079,XM_{—}114617,gen.XM_{—}114617

FIG. 2518: PRO81658

FIG. 2519: DNA325080,XM_{—}088336,gen.XM_{—}088336

FIG. 2520: PRO81659

FIG. 2521: DNA325081,XM_{—}047083,gen.XM_{—}047083

FIG. 2522: PRO81660

FIG. 2523: DNA325082,XM_{—}114618,gen.XM_{—}114618

FIG. 2524: PRO81661

FIG. 2525: DNA325083,XM_{—}050215,gen.XM_{—}050215

FIG. 2526: DNA325084,XM_{—}113531,gen.XM_{—}113531

FIG. 2527: DNA325085,NM_{—}018310,gen.NM_{—}018310

FIG. 2528: PRO81664

FIG. 2529: DNA325086,XM_{—}088294,gen.XM_{—}088294

FIG. 2530: DNA325087,XM_{—}013112,gen.XM_{—}013112

FIG. 2531: DNA325088,XM_{—}059933,gen.XM_{—}059933

FIG. 2532: PRO1108

FIG. 2533: DNA325089,XM_{—}011629,gen.XM_{—}011629

FIG. 2534: DNA325090,NM_{—}000930,gen.NM_{—}000930

FIG. 2535: PRO4

FIG. 2536: DNA325091,NM_{—}000931,gen.NM_{—}000931

FIG. 2537: PRO81668

FIG. 2538: DNA325092,NM_{—}033011,gen.NM_{—}033011

FIG. 2539: PRO81669

FIG. 2540: DNA325093,XM_{—}166063,gen.XM_{—}166063

FIG. 2541: DNA325094,NM_{—}025070,gen.NM_{—}025070

FIG. 2542: PRO81671

FIG. 2543A-B: DNA325095,XM_{—}030268,gen.XM_{—}030268

FIG. 2544: DNA325096,XM_{—}030274,gen.XM_{—}030274

FIG. 2545: PRO81673

FIG. 2546: DNA151010,NM_{—}003350,gen.NM_{—}003350

FIG. 2547: PRO12838

FIG. 2548: DNA325097,XM_{—}113540,gen.XM_{—}113540

FIG. 2549: PRO81674

FIG. 2550: DNA325098,NM_{—}006330,gen.NM_{—}006330

FIG. 2551: PRO59230

FIG. 2552: DNA325099,NM_{—}001023,gen.NM_{—}001023

FIG. 2553: PRO58263

FIG. 2554: DNA325100,XM_{—}095667,gen.XM_{—}095667

FIG. 2555: PRO81675

FIG. 2556: DNA325101,XM_{—}114640,gen.XM_{—}114640

FIG. 2557: DNA325102,XM_{—}057780,gen.XM_{—}057780

FIG. 2558: DNA325103,XM_{—}166064,gen.XM_{—}166064

FIG. 2559: DNA325104,XM_{—}088399,gen.XM_{—}088399

FIG. 2560: DNA325105,XM_{—}088401,gen.XM_{—}088401

FIG. 2561: DNA325106,XM_{—}042658,gen.XM_{—}042658

FIG. 2562: DNA325107,XM_{—}011769,gen.XM_{—}011769

FIG. 2563: DNA325108,XM_{—}044627,gen.XM_{—}044627

FIG. 2564: DNA325109,XM_{—}098761,gen.XM_{—}098761

FIG. 2565: DNA226496,NM_{—}006837,gen.NM_{—}006837

FIG. 2566: PRO36959

FIG. 2567: DNA325110,NM_{—}014294,gen.NM_{—}014294

FIG. 2568: PRO23248

FIG. 2569: DNA325111,NM_{—}000971,gen.NM_{—}000971

FIG. 2570: PRO81685

FIG. 2571: DNA325112,XM_{—}050731,gen.XM_{—}050731

FIG. 2572: DNA325113,XM_{—}088325,gen.XM_{—}088325

FIG. 2573: PRO81687

FIG. 2574: DNA325114,XM_{—}088323,gen.XM_{—}088323

FIG. 2575: DNA325115,NM_{—}001444,gen.NM_{—}001444

FIG. 2576: PRO81689

FIG. 2577: DNA325116,XM_{—}013127,gen.XM_{—}013127

FIG. 2578: PRO81690

FIG. 2579: DNA325117,XM_{—}165514,gen.XM_{—}165514

FIG. 2580: PRO81691

FIG. 2581: DNA325118,XM_{—}017816,gen.XM_{—}017816

FIG. 2582: DNA325119,XM_{—}098747,gen.XM_{—}098747

FIG. 2583: DNA325120,XM_{—}050506,gen.XM_{—}050506

FIG. 2584: DNA325121,NM_{—}024613,gen.NM_{—}024613

FIG. 2585: PRO81695

FIG. 2586: DNA325122,XM_{—}011642,gen.XM_{—}011642

FIG. 2587: PRO81696

FIG. 2588: DNA325123,NM_{—}000989,gen.NM_{—}000989

FIG. 2589: PRO11265

FIG. 2590: DNA325124,NM_{—}003406,gen.NM_{—}003406

FIG. 2591: PRO71091

FIG. 2592: DNA325125,XM_{—}011657,gen.XM_{—}011657

FIG. 2593: DNA131588,NM_{—}002568,gen.NM_{—}002568

FIG. 2594: PRO7445

FIG. 2595: DNA325126,XM_{—}018287,gen.XM_{—}018287

FIG. 2596: DNA325127,NM_{—}001568,gen.NM_{—}001568

FIG. 2597: PRO81699

FIG. 2598: DNA325128,NM_{—}003756,gen.NM_{—}003756

FIG. 2599: PRO81700

FIG. 2600A-B: DNA272050,NM_{—}006265,gen.NM_{—}006265

FIG. 2601: PRO60321

FIG. 2602: DNA325129,NM_{—}052886,gen.NM_{—}052886

FIG. 2603: PRO81701

FIG. 2604: DNA325130,XM_{—}016047,gen.XM_{—}016047

FIG. 2605: DNA325131,XM_{—}005060,gen.XM_{—}005060

FIG. 2606: DNA325132,NM_{—}005005,gen.NM_{—}005005

FIG. 2607: PRO81704

FIG. 2608: DNA325133,XM_{—}037657,gen.XM_{—}037657

FIG. 2609: DNA325134,XM_{—}029567,gen.XM_{—}029567

FIG. 2610: PRO81705

FIG. 2611: DNA325135,XM_{—}088316,gen.XM_{—}088316

FIG. 2612: DNA325136,XM_{—}051298,gen.XM_{—}051298

FIG. 2613: DNA325137,XM_{—}088370,gen.XM_{—}088370

FIG. 2614: DNA325138,NM_{—}016647,gen.NM_{—}016647

FIG. 2615: PRO23201

FIG. 2616: DNA325139,NM_{—}052963,gen.NM_{—}052963

FIG. 2617: PRO81708

FIG. 2618: DNA325140,XM_{—}049247,gen.XM_{—}049247

FIG. 2619: DNA325141,XM_{—}058968,gen.XM_{—}058968

FIG. 2620: DNA325143,NM_{—}023078,gen.NM_{—}023078

FIG. 2621: PRO81711

FIG. 2622: DNA325144,XM_{—}117487,gen.XM_{—}117487

FIG. 2623: DNA325145,XM_{—}049226,gen.XM_{—}049226

FIG. 2624: PRO81714

FIG. 2625: DNA325146,XM_{—}114613,gen.XM_{—}114613

FIG. 2626: DNA325147,XM_{—}035368,gen.XM_{—}035368

FIG. 2627: DNA325148,XM_{—}113532,gen.XM_{—}113532

FIG. 2628: DNA325149,XM_{—}088321,gen.XM_{—}088321

FIG. 2629: DNA325150,XM_{—}035373,gen.XM_{—}035373

FIG. 2630: PRO81719

FIG. 2631: DNA325151,XM_{—}035370,gen.XM_{—}035370

FIG. 2632: PRO81720

FIG. 2633: DNA325152,NM_{—}000973,gen.NM_{—}000973

FIG. 2634: PRO22907

FIG. 2635: DNA325153,NM_{—}033301,gen.NM_{—}033301

FIG. 2636: PRO22907

FIG. 2637: DNA325154,XM_{—}049421,gen.XM_{—}049421

FIG. 2638: DNA325155,XM_{—}034640,gen.XM_{—}034640

FIG. 2639: PRO81722

FIG. 2640: DNA325156,XM_{—}088550,gen.XM_{—}088550

FIG. 2641: DNA325157,XM_{—}088552,gen.XM_{—}088552

FIG. 2642: DNA325158,XM_{—}088553,gen.XM_{—}088553

FIG. 2643: PRO81725

FIG. 2644: DNA325159,XM_{—}059979,gen.XM_{—}059979

FIG. 2645: DNA325160,XM_{—}167558,gen.XM_{—}167558

FIG. 2646: DNA325161,XM_{—}039654,gen.XM_{—}039654

FIG. 2647: DNA325162,XM_{—}060006,gen.XM_{—}060006

FIG. 2648: PRO81729

FIG. 2649: DNA325163,NM_{—}001122,gen.NM_{—}001122

FIG. 2650: PRO81730

FIG. 2651: DNA325164,NM_{—}001010,gen.NM_{—}001010

FIG. 2652: PRO10824

FIG. 2653: DNA325165,NM_{—}058195,gen.NM_{—}058195

FIG. 2654: PRO81731

FIG. 2655: DNA325166,NM_{—}000077,gen.NM_{—}000077

FIG. 2656: PRO36693

FIG. 2657: DNA325167,NM_{—}058196,gen.NM_{—}058196

FIG. 2658: PRO81732

FIG. 2659: DNA325168,XM_{—}017931,gen.XM_{—}017931

FIG. 2660: DNA271847,NM_{—}001539,gen.NM_{—}001539

FIG. 2661: PRO60127

FIG. 2662: DNA270991,NM_{—}004323,gen.NM_{—}004323

FIG. 2663: PRO59321

FIG. 2664: DNA325169,NM_{—}016410,gen.NM_{—}016410

FIG. 2665: PRO81734

FIG. 2666: DNA325170,XM_{—}005543,gen.XM_{—}005543

FIG. 2667: PRO38028

FIG. 2668: DNA325171,NM_{—}001842,gen.NM_{—}001842

FIG. 2669: PRO21481

FIG. 2670: DNA226345,NM_{—}005866,gen.NM_{—}005866

FIG. 2671: PRO36808

FIG. 2672: DNA325172,XM_{—}088563,gen.XM_{—}088563

FIG. 2673: DNA325173,XM_{—}059998,gen.XM_{—}059998

FIG. 2674: PRO59579

FIG. 2675: DNA325174,NM_{—}013442,gen.NM_{—}013442

FIG. 2676: PRO9819

FIG. 2677: DNA325175,XM_{—}114661,gen.XM_{—}114661

FIG. 2678: PRO81736

FIG. 2679: DNA325176,XM_{—}048479,gen.XM_{—}048479

FIG. 2680: DNA290319,NM_{—}003289,gen.NM_{—}003289

FIG. 2681: PRO70595

FIG. 2682A-C: DNA325177,NM_{—}006289,gen.NM_{—}006289

FIG. 2683: PRO81738

FIG. 2684: DNA325178,XM_{—}048518,gen.XM_{—}048518

FIG. 2685: PRO81739

FIG. 2686: DNA325179,XM_{—}048539,gen.XM_{—}048539

FIG. 2687: PRO81740

FIG. 2688: DNA325180,XM_{—}114662,gen.XM_{—}114662

FIG. 2689: DNA325181,NM_{—}001833,gen.NM_{—}001833

FIG. 2690: PRO81742

FIG. 2691: DNA227491,NM_{—}007096,gen.NM_{—}007096

FIG. 2692: PRO37954

FIG. 2693: DNA254771,NM_{—}012203,gen.NM_{—}012203

FIG. 2694: PRO49869

FIG. 2695: DNA89242,NM_{—}000700,gen.NM_{—}000700

FIG. 2696: PRO2907

FIG. 2697: DNA325182,XM_{—}041020,gen.XM_{—}041020

FIG. 2698: PRO81743

FIG. 2699: DNA325183,XM_{—}114686,gen.XM_{—}114686

FIG. 2700: DNA325184,XM_{—}088637,gen.XM_{—}088637

FIG. 2701: DNA287216,NM_{—}021154,gen.NM_{—}021154

FIG. 2702: PRO69496

FIG. 2703: DNA288247,NM_{—}058179,gen.NM_{—}058179

FIG. 2704: PRO70011

FIG. 2705: DNA325185,XM_{—}071178,gen.XM_{—}071178

FIG. 2706: PRO81746

FIG. 2707: DNA325186,XM_{—}005490,gen.XM_{—}005490

FIG. 2708: DNA325187,NM_{—}031263,gen.NM_{—}031263

FIG. 2709: PRO81748

FIG. 2710: DNA325188,XM_{—}018006,gen.XM_{—}018006

FIG. 2711: DNA325189,XM_{—}017996,gen.XM_{—}017996

FIG. 2712: DNA325190,XM_{—}016113,gen.XM_{—}016113

FIG. 2713: PRO81751

FIG. 2714: DNA272655,NM_{—}001827,gen.NM_{—}991827

FIG. 2715: PRO60781

FIG. 2716A-B: DNA325191,NM_{—}002161,gen.NM_{—}002161

FIG. 2717: PRO81752

FIG. 2718A-B: DNA325192,NM_{—}013417,gen.NM_{—}013417

FIG. 2719: PRO81753

FIG. 2720A-B: DNA325193,XM_{—}046863,gen.XM_{—}046863

FIG. 2721: PRO81754

FIG. 2722: DNA325194,XM_{—}046836,gen.XM_{—}046836

FIG. 2723: DNA275322,NM_{—}003837,gen.NM_{—}003837

FIG. 2724: PRO63000

FIG. 2725A-B: DNA325195,XM_{—}098943,gen.XM_{—}098943

FIG. 2726: DNA325196,XM_{—}016308,gen.XM_{—}016308

FIG. 2727: DNA325197,XM_{—}005525,gen.XM_{—}005525

FIG. 2728: DNA325198,NM_{—}003389,gen.NM_{—}003389

FIG. 2729: PRO81759

FIG. 2730: DNA325199,NM_{—}033219,gen.NM_{—}033219

FIG. 2731: PRO81760

FIG. 2732: DNA325200,NM_{—}006401,gen.NM_{—}006401

FIG. 2733: PRO81761

FIG. 2734: DNA272213,NM_{—}002486,gen.NM_{—}002486

FIG. 2735: PRO60475

FIG. 2736: DNA325201,NM_{—}001333,gen.NM_{—}001333

FIG. 2737: PRO81762

FIG. 2738: DNA325202,XM_{—}116818,gen.XM_{—}116818

FIG. 2739: PRO81763

FIG. 2740: DNA254543,NM_{—}006808,gen.NM_{—}006808

FIG. 2741: PRO49648

FIG. 2742: DNA325203,XM_{—}070873,gen.XM_{—}070873

FIG. 2743: PRO81764

FIG. 2744: DNA325204,XM_{—}042788,gen.XM_{—}042788

FIG. 2745: PRO81765

FIG. 2746: DNA257309,NM_{—}032342,gen.NM_{—}032342

FIG. 2747: PRO51901

FIG. 2748: DNA325205,XM_{—}088569,gen.XM_{—}088569

FIG. 2749: PRO81766

FIG. 2750: DNA325206,XM_{—}088571,gen.XM_{—}088571

FIG. 2751: DNA271722,NM_{—}004697,gen.NM_{—}004697

FIG. 2752: PRO60006

FIG. 2753: DNA325207,NM_{—}017443,gen.NM_{—}017443

FIG. 2754: PRO81768

FIG. 2755A-C: DNA325208,XM_{—}005348,gen.XM_{—}005348

FIG. 2756: DNA325209,XM_{—}114646,gen.XM_{—}114646

FIG. 2757: DNA325210,XM_{—}038391,gen.XM_{—}038391

FIG. 2758: PRO81771

FIG. 2759A-B: DNA325211,XM_{—}045296,gen.XM_{—}045296

FIG. 2760: DNA325212,XM_{—}005365,gen.XM_{—}005365

FIG. 2761: DNA289530,NM_{—}004435,gen.NM_{—}004435

FIG. 2762: PRO70290

FIG. 2763: DNA287271,NM_{—}032799,gen.NM_{—}032799

FIG. 2764: PRO69542

FIG. 2765: DNA325213,XM_{—}026987,gen.XM_{—}026987

FIG. 2766: DNA325214,XM_{—}026985,gen.XM_{—}026985

FIG. 2767: DNA225630,NM_{—}016174,gen.NM_{—}016174

FIG. 2768: PRO36093

FIG. 2769: DNA325215,XM_{—}026968,gen.XM_{—}026968

FIG. 2770: PRO81775

FIG. 2771: DNA325216,XM_{—}026951,gen.XM_{—}026951

FIG. 2772: DNA325217,NM_{—}025072,gen.NM_{—}025072

FIG. 2773: PRO33818

FIG. 2774: DNA325218,XM_{—}033424,gen.XM_{—}033424

FIG. 2775: DNA325219,NM_{—}004957,gen.NM_{—}004957

FIG. 2776: PRO81778

FIG. 2777: DNA325220,XM_{—}033457,gen.XM_{—}033457

FIG. 2778A-B: DNA325221,XM_{—}033460,gen.XM_{—}033460

FIG. 2779: PRO81780

FIG. 2780: DNA325222,NM_{—}000976,gen.NM_{—}000976

FIG. 2781: PRO62236

FIG. 2782: DNA218841,NM_{—}012098,gen.NM_{—}012098

FIG. 2783: PRO34473

FIG. 2784A-B: DNA325223,XM_{—}052725,gen.XM_{—}052725

FIG. 2785: PRO81781

FIG. 2786: DNA325224,XM_{—}011752,gen.XM_{—}011752

FIG. 2787: DNA325225,XM_{—}026944,gen.XM_{—}026944

FIG. 2788: PRO81783

FIG. 2789: DNA325226,XM_{—}116806,gen.XM_{—}116806

FIG. 2790A-B: DNA325227,NM_{—}005347,gen.NM_{—}005347

FIG. 2791: PRO81785

FIG. 2792: DNA325228,NM_{—}005833,gen.NM_{—}005833

FIG. 2793: PRO81786

FIG. 2794: DNA325229,NM_{—}007209,gen.NM_{—}007209

FIG. 2795: PRO61897

FIG. 2796: DNA88350,NM_{—}000177,gen.NM_{—}000177

FIG. 2797: PRO2758

FIG. 2798A-B: DNA325230,XM_{—}011749,gen.XM_{—}011749

FIG. 2799: DNA325231,XM_{—}114679,gen.XM_{—}114679

FIG. 2800: DNA325232,XM_{—}087041,gen.XM_{—}087041

FIG. 2801: DNA325233,XM_{—}114678,gen.XM_{—}114678

FIG. 2802: DNA325234,XM_{—}114677,gen.XM_{—}114677

FIG. 2803: DNA325235,XM_{—}087038,gen.XM_{—}087038

FIG. 2804: DNA325236,XM_{—}059637,gen.XM_{—}059637

FIG. 2805: PRO81792

FIG. 2806: DNA325237,NM_{—}000368,gen.NM_{—}000368

FIG. 2807: PRO60115

FIG. 2808: DNA325238,XM_{—}033385,gen.XM_{—}033385

FIG. 2809A-B: DNA325239,XM_{—}033380,gen.XM_{—}033380

FIG. 2810: PRO81794

FIG. 2811: DNA325240,XM_{—}033362,gen.XM_{—}033362

FIG. 2813: DNA325241,XM_{—}059986,gen.XM_{—}059986

FIG. 2814: PRO81796

FIG. 2815A-B: DNA325242,XM_{—}033361,gen.XM_{—}033361

FIG. 2816: PRO81797

FIG. 2817A-B: DNA325243,XM_{—}033360,gen.XM_{—}033360

FIG. 2818: DNA325244,XM_{—}033359,gen.XM_{—}033359

FIG. 2819A-B: DNA325245,XM_{—}033355,gen.XM_{—}033355

FIG. 2820: DNA325246,NM_{—}014285,gen.NM_{—}014285

FIG. 2821: PRO81800

FIG. 2822: DNA325247,NM_{—}054012,gen.NM_{—}054012

FIG. 2823: PRO81801

FIG. 2824: DNA325248,XM_{—}035103,gen.XM_{—}035103

FIG. 2825: DNA325249,XM_{—}035109,gen.XM_{—}035109

FIG. 2826: DNA325250,NM_{—}000972,gen.NM_{—}000972

FIG. 2827: PRO81804

FIG. 2828: DNA325251,NM_{—}033161,gen.NM_{—}033161

FIG. 2829: PRO81805

FIG. 2830: DNA325252,NM_{—}000787,gen.NM_{—}000787

FIG. 2831: PRO81806

FIG. 2832A-B: DNA325253,XM_{—}011778,gen.XM_{—}011778

FIG. 2833: DNA325254,XM_{—}088426,gen.XM_{—}088426

FIG. 2834: DNA325255,NM_{—}002003,gen.NM_{—}002003

FIG. 2835: PRO1910

FIG. 2836: DNA325256,NM_{—}058199,gen.NM_{—}058199

FIG. 2837: PRO81809

FIG. 2838: DNA325257,XM_{—}059945,gen.XM_{—}059945

FIG. 2839: DNA325258,XM_{—}088422,gen.XM_{—}088422

FIG. 2840: PRO81811

FIG. 2841: DNA325259,XM_{—}029168,gen.XM_{—}029168

FIG. 2842: PRO81812

FIG. 2843: DNA325260,XM_{—}098913,gen.XM_{—}098913

FIG. 2844: PRO81813

FIG. 2845: DNA325261,XM_{—}114669,gen.XM_{—}114669

FIG. 2846: DNA325262,XM_{—}113564,gen.XM_{—}113564

FIG. 2847A-B: DNA325263,XM_{—}088459,gen.XM_{—}088459

FIG. 2848: PRO81815

FIG. 2849: DNA325264,XM_{—}054752,gen.XM_{—}054752

FIG. 2850: PRO81816

FIG. 2851: DNA325265,XM_{—}084270,gen.XM_{—}084270

FIG. 2852: DNA325266,XM_{—}054763,gen.XM_{—}054763

FIG. 2853: PRO81817

FIG. 2854: DNA325267,XM_{—}114655,gen.XM_{—}114655

FIG. 2855: DNA325268,XM_{—}038030,gen.XM_{—}038030

FIG. 2856: PRO59351

FIG. 2857: DNA325269,XM_{—}072526,gen.XM_{—}072526

FIG. 2858: PRO81819

FIG. 2859: DNA325270,XM_{—}059961,gen.XM_{—}059961

FIG. 2860: DNA325271,NM_{—}032928,gen.NM_{—}032928

FIG. 2861: PRO81821

FIG. 2862: DNA325272,NM_{—}014172,gen.NM_{—}014172

FIG. 2863: PRO81822

FIG. 2864: DNA325273,XM_{—}038049,gen.XM_{—}038049

FIG. 2865: PRO62069

FIG. 2866: DNA325274,XM_{—}038063,gen.XM_{—}038063

FIG. 2867: PRO81823

FIG. 2868: DNA325275,NM_{—}000954,gen.NM_{—}000954

FIG. 2869: PRO81824

FIG. 2870: DNA325276,XM_{—}088461,gen.XM_{—}088461

FIG. 2871: DNA325277,XM_{—}059966,gen.XM_{—}059966

FIG. 2872: PRO81826

FIG. 2873: DNA325278,XM_{—}114649,gen.XM_{—}114649

FIG. 2874: DNA325279,XM_{—}117519,gen.XM_{—}117519

FIG. 2875: DNA325280,XM_{—}053206,gen.XM_{—}053206

FIG. 2876: DNA325281,XM_{—}040272,gen.XM_{—}040272

FIG. 2877: PRO58939

FIG. 2878: DNA325282,XM_{—}005724,gen.XM_{—}005724

FIG. 2879: DNA325283,XM_{—}040267,gen.XM_{—}040267

FIG. 2880: PRO81831

FIG. 2881: DNA325284,XM_{—}048859,gen.XM_{—}048859

FIG. 2882: PRO62617

FIG. 2883: DNA325285,NM_{—}003739,gen.NM_{—}003739

FIG. 2884: PRO81832

FIG. 2885: DNA325286,XM_{—}060976,gen.XM_{—}060976

FIG. 2886: PRO81833

FIG. 2887: DNA325287,XM_{—}167626,gen.XM_{—}167626

FIG. 2888: PRO81834

FIG. 2889: DNA325288,XM_{—}165555,gen.XM_{—}165555

FIG. 2890: PRO81835

FIG. 2891: DNA325289,NM_{—}001494,gen.NM_{—}001494

FIG. 2892: PRO81836

FIG. 2893: DNA325290,NM_{—}032905,gen.NM_{—}032905

FIG. 2894: PRO81837

FIG. 2895: DNA325291,NM_{—}005174,gen.NM_{—}005174

FIG. 2896: PRO81838

FIG. 2897: DNA325292,XM_{—}165557,gen.XM_{—}165557

FIG. 2898: DNA325293,XM_{—}167374,gen.XM_{—}167374

FIG. 2899: DNA273759,NM_{—}006023,gen.NM_{—}006023

FIG. 2900: PRO61721

FIG. 2901: DNA325294,XM_{—}167411,gen.XM_{—}167411

FIG. 2902: DNA325295,NM_{—}031453,gen.NM_{—}031453

FIG. 2903: PRO81841

FIG. 2904: DNA325296,XM_{—}167414,gen.XM_{—}167414

FIG. 2905: PRO12851

FIG. 2906: DNA325297,XM_{—}166717,gen.XM_{—}166717

FIG. 2907: PRO81842

FIG. 2908: DNA325298,XM_{—}005100,gen.XM_{—}005100

FIG. 2909: DNA325299,XM_{—}038536,gen.XM_{—}038536

FIG. 2910A-B: DNA325300,XM_{—}084420,gen.XM_{—}084420

FIG. 2911: DNA325301,XM_{—}084429,gen.XM_{—}084429

FIG. 2912: PRO81846

FIG. 2913A-C: DNA325302,XM_{—}165551,gen.XM_{—}165551

FIG. 2914: DNA325303,XM_{—}059720,gen.XM_{—}059720

FIG. 2915: PRO81848

FIG. 2916A-B: DNA325304,NM_{—}019619,gen.NM_{—}019619

FIG. 2917: PRO81849

FIG. 2918: DNA325305,XM_{—}166665,gen.XM_{—}166665

FIG. 2919A-B: DNA325306,NM_{—}002211,gen.NM_{—}002211

FIG. 2920: PRO81851

FIG. 2921A-B: DNA325307,XM_{—}165567,gen.XM_{—}165567

FIG. 2922: DNA325308,XM_{—}166157,gen.XM_{—}166157

FIG. 2923: DNA325309,NM_{—}032023,gen.NM_{—}032023

FIG. 2924: PRO52537

FIG. 2925: DNA325310,XM_{—}165560,gen.XM_{—}165560

FIG. 2926: DNA325311,XM_{—}165563,gen.XM_{—}165563

FIG. 2927: DNA325312,XM_{—}113615,gen.XM_{—}113615

FIG. 2928: PRO81855

FIG. 2929: DNA325313,XM_{—}165890,gen.XM_{—}165890

FIG. 2930: DNA325314,XM_{—}061126,gen.XM_{—}061126

FIG. 2931: DNA325315,XM_{—}061125,gen.XM_{—}061125

FIG. 2932: PRO81858

FIG. 2933: DNA325316,XM_{—}054474,gen.XM_{—}054474

FIG. 2934: DNA325317,XM_{—}165888,gen.XM_{—}165888

FIG. 2935: DNA325318,XM_{—}054475,gen.XM_{—}054475

FIG. 2936: PRO81861

FIG. 2937: DNA325319,XM_{—}015652,gen.XM_{—}015652

FIG. 2938: PRO81862

FIG. 2939: DNA325320,XM_{—}036593,gen.XM_{—}036593

FIG. 2940: PRO81863

FIG. 2941: DNA325321,XM_{—}165891,gen.XM_{—}165891

FIG. 2942: DNA325322,XM_{—}084450,gen.XM_{—}084450

FIG. 2943: PRO81865

FIG. 2944: DNA325323,XM_{—}084385,gen.XM_{—}084385

FIG. 2945: DNA325324,NM_{—}021226,gen.NM_{—}021226

FIG. 2946: PRO81867

FIG. 2947: DNA193957,NM_{—}003055,gen.NM_{—}003055

FIG. 2948: PRO23364

FIG. 2949: DNA325325,NM_{—}032997,gen.NM_{—}032997

FIG. 2950: PRO81868

FIG. 2951: DNA287642,NM_{—}018464,gen.NM_{—}018464

FIG. 2952: PRO9902

FIG. 2953: DNA325326,XM_{—}084451,gen.XM_{—}084451

FIG. 2954: PRO81869

FIG. 2955: DNA325327,NM_{—}012207,gen.NM_{—}012207

FIG. 2956: PRO81870

FIG. 2957: DNA325328,NM_{—}024045,gen.NM_{—}024045

FIG. 2958: PRO81871

FIG. 2959: DNA325329,NM_{—}004728,gen.NM_{—}004728

FIG. 2960: PRO81872

FIG. 2961: DNA88562,NM_{—}002727,gen.NM_{—}002727

FIG. 2962: PRO2842

FIG. 2963: DNA325330,XM_{—}167395,gen.XM_{—}167395

FIG. 2964: DNA227172,NM_{—}021129,gen.NM_{—}021129

FIG. 2965: PRO37635

FIG. 2966A-B: DNA325331,XM_{—}166125,gen.XM_{—}166125

FIG. 2967: PRO81874

FIG. 2968: DNA325332,XM_{—}044354,gen.XM_{—}044354

FIG. 2969: PRO81875

FIG. 2970: DNA325333,XM_{—}032520,gen.XM_{—}032520

FIG. 2971: DNA325334,NM_{—}019058,gen.NM_{—}019058

FIG. 2972: PRO81877

FIG. 2973: DNA325335,XM_{—}045140,gen.XM_{—}045140

FIG. 2974: PRO2875

FIG. 2975: DNA325336,XM_{—}116863,gen.XM_{—}116863

FIG. 2976: DNA325337,XM_{—}032476,gen.XM_{—}032476

FIG. 2977: DNA325338,XM_{—}114894,gen.XM_{—}114894

FIG. 2978: DNA325339,NM_{—}033022,gen.NM_{—}033022

FIG. 2979: PRO81881

FIG. 2980: DNA325340,NM_{—}001026,gen.NM_{—}001026

FIG. 2981: PRO11139

FIG. 2982: DNA103421,NM_{—}003375,gen.NM_{—}003375

FIG. 2983: PRO4749

FIG. 2984A-B: DNA325341,XM_{—}166093,gen.XM_{—}166093

FIG. 2985: PRO81882

FIG. 2986: DNA304459,NM_{—}005729,gen.NM_{—}005729

FIG. 2987: PRO37073

FIG. 2988: DNA325342,XM_{—}166629,gen.XM_{—}166629

FIG. 2989: PRO81883

FIG. 2990: DNA103506,NM_{—}001157,gen.NM_{—}001157

FIG. 2991: PRO4833

FIG. 2992: DNA325343,XM_{—}016093,gen.XM_{—}016093

FIG. 2993: PRO81884

FIG. 2994: DNA325344,XM_{—}084467,gen.XM_{—}084467

FIG. 2995: PRO81885

FIG. 2996: DNA304488,NM_{—}032333,gen.NM_{—}032333

FIG. 2997: PRO71057

FIG. 2998: DNA325345,XM_{—}043589,gen.XM_{—}043589

FIG. 2999: DNA325346,XM_{—}043605,gen.XM_{—}043605

FIG. 3000: DNA325347,XM_{—}087480,gen.XM_{—}087480

FIG. 3001: PRO81887

FIG. 3002: DNA325348,NM_{—}002921,gen.NM_{—}002921

FIG. 3003: PRO81888

FIG. 3004: DNA226217,NM_{—}005271,gen.NM_{—}005271

FIG. 3005: PRO36680

FIG. 3006: DNA325349,XM_{—}089551,gen.XM_{—}089551

FIG. 3007: PRO81889

FIG. 3008: DNA287237,NM_{—}001613,gen.NM_{—}001613

FIG. 3009: PRO39648

FIG. 3010: DNA325350,NM_{—}084477,gen.XM_{—}084477

FIG. 3011: PRO69523

FIG. 3012: DNA325351,XM_{—}084480,gen.XM_{—}084480

FIG. 3013A-B: DNA325352,NM_{—}013451,gen.NM_{—}013451

FIG. 3014: PRO12813

FIG. 3015: DNA325353,XM_{—}018167,gen.XM_{—}018167

FIG. 3016: DNA325354,XM_{—}084372,gen.XM_{—}084372

FIG. 3017: DNA325355,NM_{—}020992,gen.NM_{—}020992

FIG. 3018: PRO81893

FIG. 3019: DNA325356,XM_{—}089514,gen.XM_{—}089514

FIG. 3020A-B: DNA325357,XM_{—}058343,gen.XM_{—}058343

FIG. 3021: PRO81895

FIG. 3022: DNA325358,XM_{—}058602,gen.XM_{—}058602

FIG. 3023: PRO81896

FIG. 3024A-B: DNA325359,NM_{—}015179,gen.NM_{—}015179

FIG. 3025: PRO81897

FIG. 3026: DNA325360,XM_{—}083842,gen.XM_{—}083842

FIG. 3027: PRO69473

FIG. 3028: DNA325361,XM_{—}084413,gen.XM_{—}084413

FIG. 3029: DNA325362,NM_{—}022362,gen.NM_{—}022362

FIG. 3030: PRO81899

FIG. 3031: DNA325363,NM_{—}032112,gen.NM_{—}032112

FIG. 3032: PRO81900

FIG. 3033: DNA325364,NM_{—}021830,gen.NM_{—}021830

FIG. 3034: PRO81901

FIG. 3035A-B: DNA325365,XM_{—}046743,gen.XM_{—}046743

FIG. 3036: PRO81902

FIG. 3037: DNA325366,NM_{—}013274,gen.NM_{—}013274

FIG. 3038: PRO81903

FIG. 3039: DNA325367,NM_{—}022039,gen.NM_{—}022039

FIG. 3040: PRO81904

FIG. 3041A-B: DNA325368,XM_{—}031866,gen.XM_{—}031866

FIG. 3042A-B: DNA325369,NM_{—}015062,gen.NM_{—}015062

FIG. 3043: PRO81905

FIG. 3044A-B: DNA325370,XM_{—}031890,gen.XM_{—}031890

FIG. 3045A-B: DNA325371,NM_{—}004193,gen.NM_{—}004193

FIG. 3046: PRO81907

FIG. 3047: DNA325372,NM_{—}024040,gen.NM_{—}024040

FIG. 3048: PRO81908

FIG. 3049: DNA325373,XM_{—}031949,gen.XM_{—}031949

FIG. 3050: PRO4900

FIG. 3051A-B: DNA144601,NM_{—}016169,gen.NM_{—}016169

FIG. 3052: PRO34073

FIG. 3053: DNA325374,XM_{—}005698,gen.XM_{—}005698

FIG. 3054: PRO81909

FIG. 3055: DNA325375,NM_{—}006523,gen.NM_{—}006523

FIG. 3056: PRO59043

FIG. 3057: DNA325376,XM_{—}018279,gen.XM_{—}018279

FIG. 3058A-B: DNA325377,XM_{—}005938,gen.XM_{—}005938

FIG. 3059A-B: DNA325378,XM_{—}031992,gen.XM_{—}031992

FIG. 3060: PRO81912

FIG. 3061: DNA325379,NM_{—}032747,gen.NM_{—}032747

FIG. 3062: PRO81913

FIG. 3063: DNA325380,NM_{—}005004,gen.NM_{—}005004

FIG. 3064: PRO81914

FIG. 3065: DNA325381,XM_{—}030447,gen.XM_{—}030447

FIG. 3066: DNA273521,NM_{—}002079,gen.NM_{—}002079

FIG. 3067: PRO61502

FIG. 3068A-B: DNA325382,NM_{—}032211,gen.NM_{—}032211

FIG. 3069: PRO81916

FIG. 3070: DNA325383,NM_{—}031484,gen.NM_{—}031484

FIG. 3071: PRO81917

FIG. 3072: DNA325384,XM_{—}084632,gen.XM_{—}084632

FIG. 3073: DNA325385,XM_{—}084359,gen.XM_{—}084359

FIG. 3074A-D: DNA325386,XM_{—}045667,gen.XM_{—}045667

FIG. 3075: DNA325387,XM_{—}109162,gen.XM_{—}109162

FIG. 3076: DNA227509,NM_{—}000274,gen.NM_{—}000274

FIG. 3077: PRO37972

FIG. 3078: DNA325388,XM_{—}058361,gen.XM_{—}058361

FIG. 3079: PRO81922

FIG. 3080: DNA325389,XM_{—}084505,gen.XM_{—}084505

FIG. 3081: PRO81923

FIG. 3082A-B: DNA325390,XM_{—}049795,gen.XM_{—}049795

FIG. 3083: PRO81924

FIG. 3084: DNA325391,XM_{—}058406,gen.XM_{—}058406

FIG. 3085: PRO81925

FIG. 3086: DNA325392,XM_{—}055573,gen.XM_{—}055573

FIG. 3087: PRO60991

FIG. 3088: DNA325393,XM_{—}005969,gen.XM_{—}005969

FIG. 3089: DNA325394,NM_{—}007190,gen.NM_{—}007190

FIG. 3090: PRO81926

FIG. 3091: DNA325395,NM_{—}000982,gen.NM_{—}000982

FIG. 3092: PRO81927

FIG. 3093: DNA269952,NM_{—}004725,gen.NM_{—}004725

FIG. 3094: PRO58348

FIG. 3095: DNA325396,NM_{—}024942,gen.NM_{—}024942

FIG. 3096: PRO81928

FIG. 3097: DNA325397,NM_{—}016567,gen.NM_{—}016567

FIG. 3098: PRO81929

FIG. 3099: DNA325398,NM_{—}004092,gen.NM_{—}004092

FIG. 3100: PRO81930

FIG. 3101: DNA269431,NM_{—}006659,gen.NM_{—}006659

FIG. 3102: PRO57854

FIG. 3103: DNA325399,XM_{—}005675,gen.XM_{—}005675

FIG. 3104: DNA325400,XM_{—}114862,gen.XM_{—}114862

FIG. 3105: PRO81932

FIG. 3106: DNA325401,XM_{—}088009,gen.XM_{—}088009

FIG. 3107: DNA325402,NM_{—}016526,gen.NM_{—}016526

FIG. 3108: PRO81934

FIG. 3109: DNA255696,NM_{—}021932,gen.NM_{—}021932

FIG. 3110: PRO50756

FIG. 3111: DNA325403,XM_{—}043220,gen.XM_{—}043220

FIG. 3112: PRO81935

FIG. 3113: DNA255078,NM_{—}006435,gen.NM_{—}006435

FIG. 3114: PRO50165

FIG. 3115: DNA325404,NM_{—}002339,gen.NM_{—}002339

FIG. 3116: PRO81936

FIG. 3117: DNA325405,XM_{—}028192,gen.XM_{—}028192

FIG. 3118: PRO81937

FIG. 3119: DNA325406,XM_{—}096544,gen.XM_{—}096544

FIG. 3120: DNA325407,NM_{—}000612,gen.NM_{—}000612

FIG. 3121: PRO124

FIG. 3122: DNA325408,XM_{—}084742,gen.XM_{—}084742

FIG. 3123: PRO81939

FIG. 3124: DNA325409,XM_{—}084739,gen.XM_{—}084739

FIG. 3125: DNA325410,XM_{—}058505,gen.XM_{—}058505

FIG. 3126: PRO81941

FIG. 3127: DNA325411,XM_{—}006139,gen.XM_{—}006139

FIG. 3128: PRO81942

FIG. 3129: DNA325412,XM_{—}044932,gen.XM_{—}044932

FIG. 3130: PRO81943

FIG. 3131A-B: DNA325413,XM_{—}044957,gen.XM_{—}044957

FIG. 3132: PRO81944

FIG. 3133: DNA325414,NM_{—}001909,gen.NM_{—}001909

FIG. 3134: PRO292

FIG. 3135: DNA325415,XM_{—}006475,gen.XM_{—}006475

FIG. 3136: DNA325416,XM_{—}006483,gen.XM_{—}006483

FIG. 3137: DNA325417,NM_{—}001751,gen.NM_{—}001751

FIG. 3138: PRO69635

FIG. 3139: DNA325418,XM_{—}114981,gen.XM_{—}114981

FIG. 3140: PRO81945

FIG. 3141: DNA325419,XM_{—}083852,gen.XM_{—}083852

FIG. 3142: DNA325420,NM_{—}000559,gen.NM_{—}000559

FIG. 3143: PRO81946

FIG. 3144: DNA325421,NM_{—}000184,gen.NM_{—}000184

FIG. 3145: PRO81947

FIG. 3146: DNA325422,NM_{—}005330,gen.NM_{—}005330

FIG. 3147: PRO81948

FIG. 3148: DNA325423,XM_{—}015243,gen.XM_{—}015243

FIG. 3149: DNA325424,NM_{—}015324,gen.NM_{—}015324

FIG. 3150: PRO81950

FIG. 3151: DNA325425,XM_{—}006424,gen.XM_{—}006424

FIG. 3152: DNA325426,XM_{—}113238,gen.XM_{—}113238

FIG. 3153A-C: DNA325427,XM_{—}052786,gen.XM_{—}052786

FIG. 3154: PRO81953

FIG. 3155: DNA325428,NM_{—}000990,gen.NM_{—}000990

FIG. 3156: PRO25985

FIG. 3157A-B: DNA325429,XM_{—}045750,gen.XM_{—}045750

FIG. 3158: PRO81954

FIG. 3159: DNA325430,XM_{—}058414,gen.XM_{—}058414

FIG. 3160: PRO81955

FIG. 3161A-B: DNA325431,XM_{—}049197,gen.XM_{—}049197

FIG. 3162: PRO81956

FIG. 3163A-B: DNA325432,NM_{—}001418,gen.NM_{—}001418

FIG. 3164: PRO81957

FIG. 3165: DNA325433,XM_{—}096520,gen.XM_{—}096520

FIG. 3166: PRO81958

FIG. 3167: DNA325434,XM_{—}006212,gen.XM_{—}006212

FIG. 3168: PRO81959

FIG. 3169: DNA325435,XM_{—}084527,gen.XM_{—}084527

FIG. 3170: DNA325436,XM_{—}016139,gen.XM_{—}016139

FIG. 3171: DNA325437,NM_{—}001017,gen.NM_{—}001017

FIG. 3172: PRO11262

FIG. 3173: DNA325438,NM_{—}014267,gen.NM_{—}014267

FIG. 3174: PRO81962

FIG. 3175: DNA97285,NM_{—}005566,gen.NM_{—}005566

FIG. 3176: PRO3632

FIG. 3177: DNA325439,XM_{—}115081,gen.XM_{—}115081

FIG. 3178: DNA325440,XM_{—}036339,gen.XM_{—}036339

FIG. 3179: PRO81964

FIG. 3180: DNA325441,XM_{—}084514,gen.XM_{—}084514

FIG. 3181: PRO81965

FIG. 3182: DNA325442,XM_{—}084516,gen.XM_{—}084516

FIG. 3183: DNA325443,XM_{—}084515,gen.XM_{—}084515

FIG. 3184: DNA325444,XM_{—}084517,gen.XM_{—}084517

FIG. 3185: DNA325445,XM_{—}034431,gen.XM_{—}034431

FIG. 3186: PRO11691

FIG. 3187: DNA325446,XM_{—}030326,gen.XM_{—}030326

FIG. 3188: DNA325447,NM_{—}057174,gen.NM_{—}057174

FIG. 3189: PRO81970

FIG. 3190: DNA325448,NM_{—}004813,gen.NM_{—}004813

FIG. 3191: PRO81971

FIG. 3192: DNA325449,XM_{—}167437,gen.XM_{—}167437

FIG. 3193: DNA325450,XM_{—}054856,gen.XM_{—}054856

FIG. 3194: DNA325451,XM_{—}004330,gen.XM_{—}004330

FIG. 3195: DNA325452,XM_{—}084681,gen.XM_{—}084681

FIG. 3196: DNA325453,XM_{—}006297,gen.XM_{—}006297

FIG. 3197: DNA325454,NM_{—}003646,gen.NM_{—}003646

FIG. 3198: PRO81977

FIG. 3199: DNA325455,NM_{—}004551,gen.NM_{—}004551

FIG. 3200: PRO81978

FIG. 3201: DNA325456,XM_{—}006170,gen.XM_{—}006170

FIG. 3202: DNA325457,XM_{—}037173,gen.XM_{—}037173

FIG. 3203: PRO81980

FIG. 3204: DNA150974,NM_{—}005693,gen.NM_{—}005693

FIG. 3205: PRO12224

FIG. 3206: DNA226080,NM_{—}001610,gen.NM_{—}001610

FIG. 3207: PRO36543

FIG. 3208: DNA270134,NM_{—}000107,gen.NM_{—}000107

FIG. 3209: PRO58523

FIG. 3210: DNA325458,NM_{—}016223,gen.NM_{—}016223

FIG. 3211: PRO81981

FIG. 3212: DNA325459,XM_{—}037147,gen.XM_{—}037147

FIG. 3213: PRO81982

FIG. 3214: DNA325460,XM_{—}015705,gen.XM_{—}015705

FIG. 3215: DNA272728,NM_{—}003146,gen.NM_{—}003146

FIG. 3216: PRO60847

FIG. 3217: DNA325461,XM_{—}165611,gen.XM_{—}165611

FIG. 3218: DNA287417,NM_{—}024098,gen.NM_{—}024098

FIG. 3219: PRO69674

FIG. 3220: DNA227088,NM_{—}014502,gen.NM_{—}014502

FIG. 3221: PRO37551

FIG. 3222: DNA325462,XM_{—}165610,gen.XM_{—}165610

FIG. 3223A-B: DNA325463,XM_{—}165612,gen.XM_{—}165612

FIG. 3224: DNA325464,XM_{—}166234,gen.XM_{—}166234

FIG. 3225: DNA325465,NM_{—}015533,gen.NM_{—}015533

FIG. 3226: PRO81988

FIG. 3227: DNA325466,XM_{—}166232,gen.XM_{—}166232

FIG. 3228A-B: DNA325467,XM_{—}167748,gen.XM_{—}167748

FIG. 3229: PRO81990

FIG. 3230: DNA325468,NM_{—}004739,gen.NM_{—}004739

FIG. 3231: PRO81991

FIG. 3232: DNA325469,NM_{—}014610,gen.NM_{—}014610

FIG. 3233: PRO81992

FIG. 3234: DNA325470,XM_{—}167747,gen.XM_{—}167747

FIG. 3235: PRO81993

FIG. 3236: DNA287254,NM_{—}024099,gen.NM_{—}024099

FIG. 3237: PRO69528

FIG. 3238: DNA325471,NM_{—}015853,gen.NM_{—}015853

FIG. 3239: PRO81994

FIG. 3240: DNA325472,NM_{—}032667,gen.NM_{—}032667

FIG. 3241: PRO81995

FIG. 3242: DNA325473,NM_{—}006362,gen.NM_{—}006362

FIG. 3243: PRO81996

FIG. 3244: DNA325474,XM_{—}167716,gen.XM_{—}167716

FIG. 3245: DNA75863,NM_{—}002411,gen.NM_{—}002411

FIG. 3246: PRO2018

FIG. 3247: DNA325475,XM_{—}087710,gen.XM_{—}087710

FIG. 3248: DNA325476,XM_{—}167726,gen.XM_{—}167726

FIG. 3249: DNA325477,NM_{—}004265,gen.NM_{—}004265

FIG. 3250: PRO 12878

FIG. 3251A-B: DNA325478,NM_{—}013402,gen.NM_{—}013402

FIG. 3252: PRO81999

FIG. 3253: DNA325479,NM_{—}004111,gen.NM_{—}004111

FIG. 3254: PRO69568

FIG. 3255: DNA325480,XM_{—}048286,gen.XM_{—}048286

FIG. 3256: DNA325481,NM_{—}004322,gen.NM_{—}004322

FIG. 3257: PRO20117

FIG. 3258: DNA325482,NM_{—}032989,gen.NM_{—}032989

FIG. 3259: PRO20117

FIG. 3260: DNA325483,XM_{—}011988,gen.XM_{—}011988

FIG. 3261: DNA325484,NM_{—}031472,gen.NM_{—}031472

FIG. 3262: PRO82002

FIG. 3263: DNA325485,XM_{—}037808,gen.XM_{—}037808

FIG. 3264: DNA325486,NM_{—}004074,gen.NM_{—}004074

FIG. 3265: PRO82004

FIG. 3266: DNA325487,NM_{—}017670,gen.NM_{—}017670

FIG. 3267: PRO82005

FIG. 3268: DNA325488,XM_{—}113223,gen.XM_{—}113223

FIG. 3269: DNA325489,XM_{—}045642,gen.XM_{—}045642

FIG. 3270: DNA325490,XM_{—}006533,gen.XM_{—}006533

FIG. 3271: DNA325491,XM_{—}045613,gen.XM_{—}045613

FIG. 3272: PRO59721

FIG. 3273A-B: DNA325492,XM_{—}045612,gen.XM_{—}045612

FIG. 3274: PRO82009

FIG. 3275: DNA325493,XM_{—}113224,gen.XM_{—}113224

FIG. 3276: DNA325494,XM_{—}045499,gen.XM_{—}045499

FIG. 3277: PRO82011

FIG. 3278: DNA325495,XM_{—}045525,gen.XM_{—}045525

FIG. 3279: DNA325496,NM_{—}013265,gen.NM_{—}013265

FIG. 3280: PRO82013

FIG. 3281: DNA325497,XM_{—}006529,gen.XM_{—}006529

FIG. 3282: PRO60008

FIG. 3283: DNA325498,XM_{—}053787,gen.XM_{—}053787

FIG. 3284: DNA269803,NM_{—}001667,gen.NM_{—}001667

FIG. 3285: PRO58207

FIG. 3286: DNA325499,XM_{—}115031,gen.XM_{—}115031

FIG. 3287: DNA325500,XM_{—}084702,gen.XM_{—}084702

FIG. 3288: DNA325501,XM_{—}053796,gen.XM_{—}053796

FIG. 3289: DNA325502,NM_{—}002689,gen.NM_{—}002689

FIG. 3290: PRO82018

FIG. 3291A-D: DNA325503,XM_{—}167804,gen.XM_{—}167804

FIG. 3292: PRO82019

FIG. 3293: DNA325504,XM_{—}166235,gen.XM_{—}166235

FIG. 3294: DNA325505,XM_{—}166236,gen.XM_{—}166236

FIG. 3295: DNA270721,NM_{—}006842,gen.NM_{—}006842

FIG. 3296: PRO59084

FIG. 3297: DNA189687,NM_{—}000852,gen.NM_{—}000852

FIG. 3298: PRO25845

FIG. 3299: DNA325506,NM_{—}007103,gen.NM_{—}007103

FIG. 3300: PRO58606

FIG. 3301: DNA325507,NM_{—}005851,gen.NM_{—}005851

FIG. 3302: PRO69461

FIG. 3303A-B: DNA325508,XM_{—}165598,gen.XM_{—}165598

FIG. 3304: DNA325509,NM_{—}006019,gen.NM_{—}006019

FIG. 3305: PRO82023

FIG. 3306: DNA325510,NM_{—}006053,gen.NM_{—}106053

FIG. 3307: PRO24831

FIG. 3308: DNA325511,XM_{—}166196,gen.XM_{—}166196

FIG. 3309: PRO82024

FIG. 3310: DNA325512,XM_{—}165600,gen.XM_{≠}165600

FIG. 3311A-B: DNA325513,NM_{—}053056,gen.NM_{—}053056

FIG. 3312: PRO4870

FIG. 3313: DNA103474,NM_{—}003824,gen.NM_{—}003824

FIG. 3314: PRO4801

FIG. 3315: DNA325514,XM_{—}096486,gen.XM_{—}096486

FIG. 3316A-B: DNA325515,NM_{—}003626,gen.NM_{—}003626

FIG. 3317: PRO82027

FIG. 3318A-B: DNA325516,XM_{—}167853,gen.XM_{—}167853

FIG. 3319: PRO82028

FIG. 3320: DNA325517,NM_{—}014042,gen.NM_{—}014042

FIG. 3321: PRO82029

FIG. 3322A-B: DNA325518,NM_{—}001567,gen.NM_{—}001567

FIG. 3323: PRO61238

FIG. 3324: DNA325519,XM_{—}167433,gen.XM_{—}167433

FIG. 3325: DNA325520,XM_{—}165616,gen.XM_{—}165616

FIG. 3326: DNA325521,NM_{—}032871,gen.NM_{—}032871

FIG. 3327: PRO57307

FIG. 3328: DNA325522,XM_{—}165631,gen.XM_{—}165631

FIG. 3329: DNA254186,NM_{—}014752,gen.NM_{—}014752

FIG. 3330: PRO49298

FIG. 3331: DNA325523,NM_{—}001005,gen.NM_{—}001005

FIG. 3332: PRO82032

FIG. 3333: DNA88176,NM_{—}001235,gen.NM_{—}001235

FIG. 3334: PRO2685

FIG. 3335A-B: DNA325524,XM_{—}165627,gen.XM_{—}165627

FIG. 3336: DNA325525,XM_{—}166253,gen.XM_{—}166253

FIG. 3337: DNA325526,NM_{—}001293,gen.NM_{—}001293

FIG. 3338: PRO82034

FIG. 3339: DNA325527,XM_{—}042852,gen.XM_{—}042852

FIG. 3340: PRO82035

FIG. 3341: DNA325528,XM_{—}165628,gen.XM_{—}165628

FIG. 3342A-B: DNA325529,NM_{—}080491,gen.NM_{—}080491

FIG. 3343: PRO82037

FIG. 3344A-B: DNA325530,NM_{—}012296,gen.NM_{—}012296

FIG. 3345: PRO60311

FIG. 3346: DNA325531,NM_{—}032379,gen.NM_{—}032379

FIG. 3347: PRO82038

FIG. 3348: DNA325532,NM_{—}007173,gen.NM_{—}007173

FIG. 3349: DNA325533,XM_{—}166239,gen.XM_{—}166239

FIG. 3350: DNA325534,XM_{—}084610,gen.XM_{—}084610

FIG. 3351: PRO82040

FIG. 3352: DNA325535,XM_{—}058450,gen.XM_{—}058450

FIG. 3353: DNA325536,XM_{—}084601,gen.XM_{—}094601

FIG. 3354: PRO82042

FIG. 3355A-B: DNA325537,XM_{—}006464,gen.XM_{—}006464

FIG. 3356: PRO82043

FIG. 3357: DNA325538,0M 084570,gen.XM_{—}084570

FIG. 3358: DNA325539,XM_{—}051435,gen.XM_{—}051435

FIG. 3359: DNA325540,NM_{—}001467,gen.NM_{—}001467

FIG. 3360: PRO82045

FIG. 3361: DNA325541,NM_{—}001028,gen.NM_{—}001028

FIG. 3362: PRO82046

FIG. 3363: DNA325542,XM_{—}113230,gen.XM_{—}113230

FIG. 3364: DNA325543,XM_{—}115062,gen.XM_{—}115062

FIG. 3365: DNA325544,XM_{—}115063,gen.XM_{—}115063

FIG. 3366: DNA325545,XM_{—}113229,gen.XM_{—}113229

FIG. 3367A-B: DNA325546,XM_{—}051489,gen.XM_{—}051489

FIG. 3368: PRO82050

FIG. 3369: DNA325547,NM_{—}022003,gen.NM_{—}022003

FIG. 3370: PRO82051

FIG. 3371: DNA325548,XM_{—}006432,gen.XM_{—}006432

FIG. 3372: PRO82052

FIG. 3373: DNA325549,XM_{—}051716,gen.XM_{—}051716

FIG. 3374: DNA325550,NM_{—}025164,gen.NM_{—}025164

FIG. 3375: PRO82054

FIG. 3376: DNA225752,NM_{—}000039,gen.NM_{—}000039

FIG. 3377: PRO36215

FIG. 3378: DNA325551,XM_{—}052113,gen.XM_{—}052113

FIG. 3379: PRO82055

FIG. 3380: DNA271324,NM_{—}006169,gen.NM_{—}006169

FIG. 3381: PRO59629

FIG. 3382: DNA325552,XM_{—}084658,gen.XM_{—}084658

FIG. 3383: PRO82056

FIG. 3384: DNA325553,NM_{—}000795,gen.NM_{—}000795

FIG. 3385: PRO12448

FIG. 3386: DNA325554,NM_{—}017868,gen.NM_{—}017868

FIG. 3387: PRO82057

FIG. 3388: DNA325555,XM_{—}084654,gen.XM_{—}084654

FIG. 3389: PRO82058

FIG. 3390: DNA272413,NM_{—}003002,gen.NM_{—}003002

FIG. 3391: PRO60666

FIG. 3392: DNA271843,NM_{—}004398,gen.NM_{—}004398

FIG. 3393: PRO60123

FIG. 3394: DNA325556,XM_{—}017369,gen.XM_{—}017369

FIG. 3395: DNA325557,NM_{—}032299,gen.NM_{—}032299

FIG. 3396: PRO82060

FIG. 3397: DNA325558,XM_{—}055369,gen.XM_{—}055369

FIG. 3398: DNA325559,XM_{—}051430,gen.XM_{—}051430

FIG. 3399: DNA325560,XM_{—}006467,gen.XM_{—}006467

FIG. 3400: DNA325561,XM_{—}113226,gen.XM_{—}113226

FIG. 3401: DNA325562,XM_{—}165592,gen.XM_{—}165592

FIG. 3402: PRO82064

FIG. 3403: DNA325563,XM_{—}166181,gen.XM_{—}166181

FIG. 3404: DNA325564,XM_{—}052862,gen.XM_{—}052862

FIG. 3405: PRO82066

FIG. 3406: DNA325565,XM_{—}166177,gen.XM_{—}166177

FIG. 3407: DNA325566,XM_{—}165571,gen.XM_{—}165571

FIG. 3408: PRO82068

FIG. 3409: DNA325567,XM_{—}166174,gen.XM_{—}166174

FIG. 3410: PRO82069

FIG. 3411: DNA325568,NM_{—}001274,gen.NM_{—}001274

FIG. 3412: PRO12187

FIG. 3413: DNA325569,XM_{—}165586,gen.XM_{—}165586

FIG. 3414: DNA325570,XM_{—}165584,gen.XM_{—}165584

FIG. 3415: DNA257965,NM_{—}032873,gen.NM_{—}032873

FIG. 3416: PRO52492

FIG. 3417: DNA325571,XM_{—}167780,gen.XM_{—}167780

FIG. 3418: DNA325572,XM_{—}166743,gen.XM_{—}166743

FIG. 3419: PRO82072

FIG. 3420: DNA325573,NM_{—}012101,gen.NM_{—}012101

FIG. 3421: PRO82073

FIG. 3422: DNA325574,NM_{—}058193,gen.NM_{—}058193

FIG. 3423: PRO82074

FIG. 3424: DNA325575,XM_{—}084522,gen.XM_{—}084522

FIG. 3425: PRO82075

FIG. 3426: DNA325576,XM_{—}091786,gen.XM_{—}091786

FIG. 3427: DNA325577,XM_{—}165390,gen.XM_{—}165390

FIG. 3428: DNA325578,XM_{—}084525,gen.XM_{—}084525

FIG. 3429A-B: DNA325579,XM_{—}010494,gen.XM_{—}010494

FIG. 3430A-B: DNA325580,NM_{—}015064,gen.NM_{—}015064

FIG. 3431: PRO82078

FIG. 3432: DNA325581,NM_{—}030775,gen.NM_{—}030775

FIG. 3433: PRO71031

FIG. 3434: DNA297398,NM_{—}032642,gen.NM_{—}032642

FIG. 3435: PRO71031

FIG. 3436: DNA325582,XM_{—}017080,gen.XM_{—}017080

FIG. 3437: DNA325583,XM_{—}113739,gen.XM_{—}113739

FIG. 3438: PRO82080

FIG. 3439: DNA325584,NM_{—}002014,gen.NM_{—}002014

FIG. 3440: PRO59262

FIG. 3441: DNA325585,XM_{—}096661,gen.XM_{—}096661

FIG. 3442: DNA325586,NM_{—}018463,gen.NM_{—}018463

FIG. 3443: PRO82082

FIG. 3444: DNA325587,NM_{—}021953,gen.NM_{—}021953

FIG. 3445: PRO82083

FIG. 3446: DNA325588,NM_{—}031465,gen.NM_{—}031465

FIG. 3447: PRO82084

FIG. 3448: DNA325589,NM_{—}005002,gen.NM_{—}005002

FIG. 3449: PRO82085

FIG. 3450: DNA325590,XM_{—}033227,gen.XM_{—}033227

FIG. 3451: DNA325591,XM_{—}116926,gen.XM_{—}116926

FIG. 3452: DNA88114,NM_{—}001734,gen.NM_{—}001734

FIG. 3453: PRO2660

FIG. 3454: DNA325592,XM_{—}058574,gen.XM_{—}058574

FIG. 3455: DNA325593,NM_{—}007273,gen.NM_{—}007273

FIG. 3456: PRO36970

FIG. 3457A-B: DNA325594,XM_{—}032588,gen.XM_{—}032588

FIG. 3458: DNA325595,NM_{—}001975,gen.NM_{—}001975

FIG. 3459: PRO38010

FIG. 3460: DNA325596,NM_{—}000365,gen.NM_{—}000365

FIG. 3461: PRO69549

FIG. 3462: DNA325597,XM_{—}032614,gen.XM_{—}032614

FIG. 3463: DNA325598,NM_{—}002075,gen.NM_{—}002075

FIG. 3464: PRO82091

FIG. 3465: DNA325599,XM_{—}165910,gen.XM_{—}165910

FIG. 3466: DNA151827,NM_{—}005439,gen.NM_{—}005439

FIG. 3467: PRO12902

FIG. 3468A-B: DNA254624,NM_{—}001273,gen.NM_{—}001273

FIG. 3469: PRO49726

FIG. 3470: DNA325600,NM_{—}015438,gen.NM_{—}015438

FIG. 3471: PRO82093

FIG. 3472: DNA325601,XM_{—}033263,gen.XM_{—}033263

FIG. 3473: DNA225632,NM_{—}002046,gen.NM_{—}002046

FIG. 3474: PRO36095

FIG. 3475A-B: DNA325602,XM_{—}006958,gen.XM_{—}006958

FIG. 3476: DNA83180,NM_{—}002342,gen.NM_{—}002342

FIG. 3477: PRO2622

FIG. 3478: DNA103514,NM_{—}001038,gen.NM_{—}001038

FIG. 3479: PRO4841

FIG. 3480: DNA188396,NM_{—}001065,gen.NM_{—}001065

FIG. 3481: PRO21924

FIG. 3482A-C: DNA325603,XM_{—}006947,gen.XM_{—}006947

FIG. 3483A-B: DNA325604,XM_{—}006936,gen.XM_{—}006936

FIG. 3484: PRO82097

FIG. 3485A-B: DNA325605,XM_{—}006925,gen.XM_{—}006925

FIG. 3486: DNA325606,XM_{—}096630,gen.XM_{—}096630

FIG. 3487: PRO82099

FIG. 3488: DNA325607,XM_{—}084901,gen.XM_{—}084901

FIG. 3489: DNA226028,NM_{—}002355,gen.NM_{—}002355

FIG. 3490: PRO36491

FIG. 3491: DNA325608,XM_{—}031807,gen.XM_{—}031807

FIG. 3492: PRO82101

FIG. 3493A-B: DNA325609,XM_{—}049663,gen.XM_{—}049663

FIG. 3494: DNA325610,XM_{—}012159,gen.XM_{—}012159

FIG. 3495: DNA325611,XM_{—}084922,gen.XM_{—}084922

FIG. 3496: DNA325612,NM_{—}031289,gen.NM_{—}031289

FIG. 3497: PRO82104

FIG. 3498: DNA226771,NM_{—}003979,gen.NM_{—}003979

FIG. 3499: PRO37234

FIG. 3500: DNA325613,XM_{—}084918,gen.XM_{—}084918

FIG. 3501: DNA325614,NM_{—}007178,gen.NM_{—}007178

FIG. 3502: PRO82106

FIG. 3503: DNA325615,XM_{—}041100,gen.XM_{—}041100

FIG. 3504A-B: DNA325616,XM_{—}058567,gen.XM_{—}058567

FIG. 3505: PRO82107

FIG. 3506A-B: DNA325617,XM_{—}166605,gen.XM_{—}166605

FIG. 3507: DNA325618,XM_{—}029805,gen.XM_{—}029805

FIG. 3508: PRO82109

FIG. 3509: DNA325619,NM_{—}005889,gen.NM_{—}005889

FIG. 3510: PRO82110

FIG. 3511: DMA256072,NM_{—}001644,gen.NM_{—}001644

FIG. 3512: PRO51121

FIG. 3513: DNA325620,NM_{—}018686,gen.NM_{—}018686

FIG. 3514: PRO82111

FIG. 3515: DNA325621,XM_{—}084770,gen.XM_{—}084770

FIG. 3516: PRO82112

FIG. 3517: DNA325622,NM_{—}018048,gen.NM_{—}018048

FIG. 3518: PRO82113

FIG. 3519: DNA325623,XM_{—}113730,gen.XM_{—}113730

FIG. 3520: DNA150978,NM_{—}007244,gen.NM_{—}007244

FIG. 3521: PRO11601

FIG. 3522: DNA325624,NM_{—}006250,gen.NM_{—}006250

FIG. 3523: PRO82115

FIG. 3524: DNA79313,NM_{—}005042,gen.NM_{—}005042

FIG. 3525: PRO2555

FIG. 3526: DNA150997,NM_{—}004982,gen.NM_{—}004982

FIG. 3527: PRO12573

FIG. 3528: DNA325625,XM_{—}050074,gen.XM_{—}050074

FIG. 3529: DNA325626,NM_{—}024854,gen.NM_{—}024854

FIG. 3530: PRO82117

FIG. 3531: DNA325627,XM_{—}084807,gen.XM_{—}084807

FIG. 3532: DNA325628,XM_{—}165906,gen.XM_{—}165906

FIG. 3533A-B: DNA325629,XM_{—}038659,gen.XM_{—}038659

FIG. 3534: PRO82120

FIG. 3535: DNA325630,XM_{—}006694,gen.XM_{—}006694

FIG. 3536: DNA325631,XM_{—}006748,gen.XM_{—}006748

FIG. 3537: PRO82122

FIG. 3538: DNA325632,XM_{—}016640,gen.XM_{—}016640

FIG. 3539: DNA325633,XM_{—}096146,gen.XM_{—}096146

FIG. 3540A-B: DNA325634,XM_{—}084841,gen.XM_{—}084841

FIG. 3541: PRO82125

FIG. 3542: DNA325635,XM_{—}090218,gen.XM_{—}090218

FIG. 3543: DNA325636,XM_{—}012272,gen.XM_{—}012272

FIG. 3544: PRO82127

FIG. 3545A-B: DNA325637,XM_{—}056481,gen.XM_{—}056481

FIG. 3546: DNA325638,NM_{—}006262,gen.NM_{—}006262

FIG. 3547: PRO82129

FIG. 3548: DNA325639,NM_{—}018113,gen.NM_{—}018113

FIG. 3549: PRO82130

FIG. 3550: DNA271344,NM_{—}001659,gen.NM_{—}001659

FIG. 3551: PRO59647

FIG. 3552: DNA325640,NM_{—}017822,gen.NM_{—}017822

FIG. 3553: PRO82131

FIG. 3554A-E: DNA325641,XM_{—}028760,gen.XM_{—}028760

FIG. 3555: DNA272379,NM_{—}002733,gen.NM_{—}002733

FIG. 3556: PRO60634

FIG. 3557: DNA325642,XM_{—}084866,gen.XM_{—}084866

FIG. 3558: PRO82133

FIG. 3559: DNA325643,XM_{—}006826,gen.XM_{—}006826

FIG. 3560: DNA325644,XM_{—}113719,gen.XM_{—}113719

FIG. 3561: DNA325645,XM_{—}028662,gen.XM_{—}028662

FIG. 3562: DNA325646,XM_{—}035497,gen.XM_{—}035497

FIG. 3563: PRO82137

FIG. 3564: DNA325647,XM_{—}035490,gen.XM_{—}035490

FIG. 3565: PRO82138

FIG. 3566: DNA325648,NM_{—}013277,gen.NM_{—}013277

FIG. 3567: PRO82139

FIG. 3568: DNA325649,NM_{—}003076,gen.NM_{—}003076

FIG. 3569: PRO82140

FIG. 3570: DNA325650,XM_{—}115117,gen.XM_{—}115117

FIG. 3571: DNA325651,XM_{—}035485,gen.XM_{—}035485

FIG. 3572A-B: DNA325652,NM_{—}016357,gen.NM_{—}016357

FIG. 3573: PRO82143

FIG. 3574: DNA325653,NM_{—}005171,gen.NM_{—}005171

FIG. 3575: PRO60924

FIG. 3576: DNA325654,NM_{—}014033,gen.NM_{—}014033

FIG. 3577: PRO4348

FIG. 3578: DNA325655,XM_{—}096620,gen.XM_{—}096620

FIG. 3579: DNA325656,XM_{—}165905,gen.XM_{—}165905

FIG. 3580: DNA325657,XM_{—}015481,gen.XM_{—}015481

FIG. 3581: DNA325658,XM_{—}049148,gen.XM_{—}049148

FIG. 3582: DNA325659,XM_{—}084885,gen.XM_{—}084885

FIG. 3583: DNA325660,XM_{—}084884,gen.XM_{—}084884

FIG. 3584: DNA325661,XM_{—}113726,gen.XM_{—}113726

FIG. 3585: DNA325662,XM_{—}015476,gen.XM_{—}015476

FIG. 3586: DNA325663,XM_{—}049141,gen.XM_{—}049141

FIG. 3587: PRO82152

FIG. 3588: DNA227191,NM_{—}021934,gen.NM_{—}021934

FIG. 3589: PRO37654

FIG. 3590: DNA325664,XM_{—}083868,gen.XM_{—}083868

FIG. 3591: DNA270458,NM_{—}002273,gen.NM_{—}002273

FIG. 3592: PRO58837

FIG. 3593: DNA227092,NM_{—}000224,gen.NM_{—}000224

FIG. 3594: PRO37555

FIG. 3595: DNA325665,XM_{—}029728,gen.XM_{—}029728

FIG. 3596: DNA325666,XM_{—}015468,gen.XM_{—}015468

FIG. 3597: PRO82155

FIG. 3598: DNA325667,XM_{—}012162,gen.XM_{—}012162

FIG. 3599: DNA325668,XM_{—}084789,gen.XM_{—}084789

FIG. 3600: DNA196351,NM_{—}002178,gen.NM_{—}002178

FIG. 3601: PRO3449

FIG. 3602A-B: DNA325669,XM_{—}29631,gen.XM_{—}029631

FIG. 3603: PRO82158

FIG. 3604: DNA325670,NM_{—}015665,gen.NM_{—}015665

FIG. 3605: PRO82159

FIG. 3606: DNA325671,NM_{—}014311,gen.NM_{—}014311

FIG. 3607: PRO82160

FIG. 3608: DNA325672,XM_{—}096606,gen.XM_{—}096606

FIG. 3609: PRO82161

FIG. 3610: DNA325673,NM_{—}018457,gen.NM_{—}018457

FIG. 3611: PRO82162

FIG. 3612: DNA325674,NM_{—}031157,gen.NM_{—}031157

FIG. 3613: PRO82163

FIG. 3614: DNA325675,NM_{—}004178,gen.NM_{—}004178

FIG. 3615: PRO82164

FIG. 3616: DNA325676,NM_{—}134323,gen.NM_{—}134323

FIG. 3617: PRO82165

FIG. 3618: DNA325677,NM_{—}134324,gen.NM_{—}134324

FIG. 3619: PRO82166

FIG. 3620: DNA290294,NM_{—}005016,gen.NM_{—}005016

FIG. 3621: PRO70453

FIG. 3622: DNA325678,NM_{—}031989,gen.NM_{—}031989

FIG. 3623: PRO82167

FIG. 3624: DNA325679,XM_{—}028643,gen.XM_{—}028643

FIG. 3625: PRO82168

FIG. 3626: DNA325680,XM_{—}006710,gen.XM_{—}006710

FIG. 3627: PRO82169

FIG. 3628: DNA227094,NM_{—}005594,gen.NM_{—}005594

FIG. 3629: PRO37557

FIG. 3630: DNA325681,XM_{—}084824,gen.XM_{—}084824

FIG. 3631: DNA304783,NM_{—}014255,gen.NM_{—}014255

FIG. 3632: PRO4426

FIG. 3633: DNA325682,XM_{—}165903,gen.XM_{—}165903

FIG. 3634: DNA325683,XM_{—}115140,gen.XM_{—}115140

FIG. 3635: DNA325684,XM_{—}113712,gen.XM_{—}113712

FIG. 3636: DNA325685,NM_{—}006601,gen.NM_{—}006601

FIG. 3637: PRO82174

FIG. 3638: DNA325686,XM_{—}012182,gen.XM_{—}012182

FIG. 3639: PRO82175

FIG. 3640: DNA325687,XM_{—}048943,gen.XM_{—}048943

FIG. 3641: DNA325688,XM_{—}053164,gen.XM_{—}053164

FIG. 3642: DNA325689,XM_{—}048991,gen.XM_{—}048991

FIG. 3643: DNA325690,NM_{—}024068,gen.NM_{—}024068

FIG. 3644: PRO82179

FIG. 3645A-B: DNA325691,XM_{—}056346,gen.XM_{—}056346

FIG. 3646: DNA325692,NM_{—}021019,gen.NM_{—}021019

FIG. 3647: PRO82181

FIG. 3648: DNA325693,NM_{—}079423,gen.NM_{—}079423

FIG. 3649: PRO82182

FIG. 3650: DNA325694,NM_{—}079425,gen.NM_{—}079425

FIG. 3651: PRO82183

FIG. 3652: DNA325695,XM_{—}049048,gen.XM_{—}049048

FIG. 3653: PRO82184

FIG. 3654: DNA325696,NM_{—}021104,gen.NM_{—}021104

FIG. 3655: PRO11213

FIG. 3656: DNA325697,NM_{—}001029,gen.NM_{—}001029

FIG. 3657: PRO10838

FIG. 3658: DNA325698,XM_{—}001482,gen.XM_{—}001482

FIG. 3659: DNA325699,XM_{—}049150,gen.XM_{—}049150

FIG. 3660: DNA325700,NM_{—}006928,gen.NM_{—}006928

FIG. 3661: PRO2846

FIG. 3662: DNA325701,XM_{—}056353,gen.XM_{—}056353

FIG. 3663: DNA325702,NM_{—}001780,gen.NM_{—}001780

FIG. 3664: PRO283

FIG. 3665: DNA325703,NM_{—}031479,gen.NM_{—}031479

FIG. 3666: PRO21773

FIG. 3667A-: DNA137231,NM_{—}005269,gen.NM_{—}005269

FIG. 3668: PRO9112

FIG. 3669: DNA325704,NM_{—}004990,gen.NM_{—}004990

FIG. 3670: PRO82188

FIG. 3671: DNA325705,XM_{—}058528,gen.XM_{—}058528

FIG. 3672: DNA325706,XM_{—}084801,gen.XM_{—}084801

FIG. 3673: PRO82190

FIG. 3674: DNA325707,XM_{—}048603,gen.XM_{—}048603

FIG. 3675: PRO82191

FIG. 3676: DNA325708,NM_{—}133483,gen.NM_{—}133483

FIG. 3677: PRO82192

FIG. 3678: DNA79101,NM_{—}006812,gen.NM_{—}006812

FIG. 3679: PRO2549

FIG. 3680: DNA325709,XM_{—}096566,gen.XM_{—}096566

FIG. 3681: DNA325710,NM_{—}005981,gen.NM_{—}005981

FIG. 3682: PRO4666

FIG. 3683: DNA325711,NM_{—}000075,gen.NM_{—}000075

FIG. 3684: PRO4873

FIG. 3685: DNA325712,NM_{—}052984,gen.NM_{—}052984

FIG. 3686: PRO82194

FIG. 3687: DNA325713,NM_{—}000785,gen.NM_{—}000785

FIG. 3688: PRO58440

FIG. 3689: DNA325714,NM_{—}005371,gen.NM_{—}005371

FIG. 3690: PRO82195

FIG. 3691: DNA325715,NM_{—}023032,gen.NM_{—}023032

FIG. 3692: PRO82196

FIG. 3693: DNA325716,NM_{—}023033,gen.NM_{—}023033

FIG. 3694: PRO82197

FIG. 3695: DNA325717,NM_{—}005726,gen.NM_{—}005726

FIG. 3696: PRO82198

FIG. 3697: DNA325718,NM_{—}006576,gen.NM_{—}006576

FIG. 3698: PRO82199

FIG. 3699A-B: DNA325719,XM_{—}96038,gen.XM_{—}96038

FIG. 3700: DNA325720,XM_{—}056681,gen.XM_{—}056681

FIG. 3701: PRO82201

FIG. 3702: DNA325721,XM_{—}084909,gen.XM_{—}084909

FIG. 3703: PRO82202

FIG. 3704: DNA325722,XM_{—}004098,gen.XM_{—}004098

FIG. 3705: DNA325723,XM_{—}084912,gen.XM_{—}084912

FIG. 3706: PRO82204

FIG. 3707: DNA325724,XM_{—}040221,gen.XM_{—}040221

FIG. 3708: DNA325725,XM_{—}016605,gen.XM_{—}016605

FIG. 3709: PRO82206

FIG. 3710: DNA325726,XM_{—}017508,gen.XM_{—}017508

FIG. 3711: PRO82207

FIG. 3712: DNA325727,NM_{—}032338,gen.NM_{—}032338

FIG. 3713: PRO82208

FIG. 3714A-B: DNA325728,XM_{—}052460,gen.XM_{—}052460

FIG. 3715: DNA325729,XM_{—}083866,gen.XM_{—}083866

FIG. 3716: PRO82210

FIG. 3717: DNA304694,NM_{—}020401,gen.NM_{—}020401

FIG. 3718: PRO71120

FIG. 3719: DNA325730,XM_{—}052474,gen.XM_{—}052474

FIG. 3720: DNA227474,NM_{—}015646,gen.NM_{—}015646

FIG. 3721: PRO37937

FIG. 3722: DNA32573 1,XM_{—}053952,gen.XM_{—}053952

FIG. 3723: PRO82212

FIG. 3724: DNA227171,NM_{—}014515,gen.NM_{—}014515

FIG. 3725: PRO37634

FIG. 3726: DNA325732,XM_{—}046041,gen.XM_{—}046041

FIG. 3727: DNA271492,NM_{—}006530,gen.NM_{—}006530

FIG. 3728: PRO59785

FIG. 3729: DNA226014,NM_{—}000239,gen.NM_{—}000239

FIG. 3730: PRO36477

FIG. 3731: DNA325733,XM_{—}084645,gen.XM_{—}084645

FIG. 3732A-B: DNA325734,XM_{—}039395,gen.XM_{—}039395

FIG. 3733: PRO82213

FIG. 3734: DNA325736,XM_{—}040644,gen.XM_{—}040644

FIG. 3735: PRO82214

FIG. 3736A-B: DNA325737,XM_{—}006578,gen.XM_{—}006578

FIG. 3737: DNA325738,XM_{—}038308,gen.XM_{—}038308

FIG. 3738: PRO82215

FIG. 3739: DNA325739,XM_{—}096597,gen.XM_{—}096597

FIG. 3740: DNA325740,NM_{—}001920,gen.NM_{—}001920

FIG. 3741: PRO2841

FIG. 3742: DNA325741,NM_{—}133503,gen.NM_{—}133503

FIG. 3743: PRO2841

FIG. 3744: DNA325742,NM_{—}133504,gen.NM_{—}133504

FIG. 3745: PRO82218

FIG. 3746: DNA325743,NM_{—}133505,gen.NM_{—}133505

FIG. 3747: PRO82219

FIG. 3748: DNA325744,NM_{—}133507,gen.NM_{—}133507

FIG. 3749: PRO82220

FIG. 3750: DNA325745,NM_{—}133506,gen.NM_{—}133506

FIG. 3751: PRO82221

FIG. 3752: DNA325746,NM_{—}002345,gen.NM_{—}002345

FIG. 3753: PRO9987

FIG. 3754: DNA325747,XM_{—}167518,gen.XM_{—}167518

FIG. 3755: DNA325748,XM_{—}052542,gen.XM_{—}052542

FIG. 3756: PRO82223

FIG. 3757: DNA325749,NM_{—}003877,gen.NM_{—}003877

FIG. 3758: PRO12839

FIG. 3759: DNA325750,XM_{—}012219,gen.XM_{—}012219

FIG. 3760: PRO69473

FIG. 3761: DNA325751,XM_{—}012145,gen.XM_{—}012145

FIG. 3762: PRO82224

FIG. 3763: DNA274361,NM_{—}000895,gen.NM_{—}000895

FIG. 3764: PRO62273

FIG. 3765: DNA325752,XM_{—}006887,gen.XM_{—}006887

FIG. 3766: DNA325753,XM_{—}006589,gen.XM_{—}006589

FIG. 3767: DNA325754,XM_{—}090458,gen.XM_{—}090458

FIG. 3768: PRO82227

FIG. 3769: DNA325755,XM_{—}052641,gen.XM_{—}052641

FIG. 3770: PRO82228

FIG. 3771A-B: DNA325756,XM_{—}049211,gen.XM_{—}049211

FIG. 3772: DNA325757,XM_{—}049201,gen.XM_{—}049201

FIG. 3773: DNA325758,XM_{—}058556,gen.XM_{—}058556

FIG. 3774: DNA325759,XM_{—}083864,gen.XM_{—}083864

FIG. 3775: DNA325760,XM_{—}062437,gen.XM_{—}062437

FIG. 3776: PRO82232

FIG. 3777: DNA254777,NM_{—}014325,gen.NM_{—}014325

FIG. 3778: PRO49875

FIG. 3779: DNA325761,XM_{—}090413,gen.XM_{—}090413

FIG. 3780: PRO82233

FIG. 3781: DNA325762,NM_{—}000970,gen.NM_{—}000970

FIG. 3782: PRO82234

FIG. 3783: DNA325763,XM_{—}084800,gen.XM_{—}084800

FIG. 3784: PRO82235

FIG. 3785: DNA325764,NM_{—}006817,gen.NM_{—}006817

FIG. 3786: PRO70694

FIG. 3787A-C: DNA325765,XM_{—}083892,gen.XM_{—}083892

FIG. 3788A-B: DNA325766,XM_{—}084941,gen.NM_{—}084941

FIG. 3789: PRO82237

FIG. 3790A-B: DNA325767,NM_{—}057169,gen.NM_{—}057169

FIG. 3791: PRO82238

FIG. 3792A-B: DNA325768,NM_{—}014776,gen.NM_{—}014776

FIG. 3793: PRO82239

FIG. 3794: DNA325769,NM_{—}032904,gen.NM_{—}032904

FIG. 3795: PRO82240

FIG. 3796A-B: DNA325770,XM_{—}007003,gen.XM_{—}007003

FIG. 3797: DNA325771,XM_{—}007002,gen.XM_{—}007002

FIG. 3798: DNA325772,XM_{—}056996,gen.XM_{—}056996

FIG. 3799: PRO82243

FIG. 3800: DNA325773,XM_{—}084946,gen.XM_{—}084946

FIG. 3801: PRO82244

FIG. 3802: DNA325775,XM_{—}027102,gen.XM_{—}027102

FIG. 3803: PRO82245

FIG. 3804: DNA325776,XM_{—}084948,gen.XM_{—}084948

FIG. 3805: DNA325777,NM_{—}007062,gen.NM_{—}007062

FIG. 3806: PRO82247

FIG. 3807: DNA325778,NM_{—}006825,gen.NM_{—}006825

FIG. 3808: PRO82248

FIG. 3809: DNA325779,XM_{—}115197,gen.XM_{—}115197

FIG. 3810: DNA325780,NM_{—}017901,gen.NM_{—}017901

FIG. 3811: PRO82250

FIG. 3812: DNA325781,NM_{—}032814,gen.NM_{—}032814

FIG. 3813: PRO82252

FIG. 3814: DNA325782,XM_{—}084889,gen.XM_{—}084889

FIG. 3815: PRO82253

FIG. 3816: DNA325783,NM_{—}002567,gen.NM_{—}002567

FIG. 3817: PRO59001

FIG. 3818: DNA325784,XM_{—}084808,gen.XM_{—}084808

FIG. 3819: DNA325785,XM_{—}096572,gen.XM_{—}096572

FIG. 3820: PRO82255

FIG. 3821: DNA325786,XM_{—}045010,gen.XM_{—}045010

FIG. 3822: PRO82256

FIG. 3823: DNA270677,NM_{—}014868,gen.NM_{—}014868

FIG. 3824: PRO59042

FIG. 3825: DNA325787,XM_{—}052893,gen.XM_{—}052893

FIG. 3826A-B: DNA325788,XM_{—}045802,gen.XM_{—}045802

FIG. 3827: DNA302016,NM_{—}001002,gen.NM_{—}001002

FIG. 3828: PRO70989

FIG. 3829: DNA325789,NM_{—}053275,gen.NM_{—}053275

FIG. 3830: PRO70989

FIG. 3831: DNA325790,NM_{—}006253,gen.NM_{—}006253

FIG. 3832: PRO82259

FIG. 3833: DNA325791,XM_{—}045187,gen.XM_{—}045187

FIG. 3834: DNA325792,XM_{—}045963,gen.XM_{—}045963

FIG. 3835: DNA325793,XM_{—}006595,gen.XM_{—}006595

FIG. 3836: DNA325794,XM_{—}012124,gen.XM_{—}012124

FIG. 3837: DNA325795,NM_{—}002813,gen.NM_{—}002813

FIG. 3838: PRO82263

FIG. 3839: DNA325796,NM_{—}019887,gen.NM_{—}019887

FIG. 3840: PRO69471

FIG. 3841A-B: DNA325797,XM_{—}038791,gen.XM_{—}038791

FIG. 3842: PRO82264

FIG. 3843: DNA325798,NM_{—}016638,gen.NM_{—}016638

FIG. 3844: PRO82265

FIG. 3845: DNA325799,XM_{—}116913,gen.XM_{—}116913

FIG. 3846: PRO82266

FIG. 3847: DNA325800,NM_{—}006815,gen.NM_{—}006815

FIG. 3848: PRO4793

FIG. 3849: DNA325801,XM_{—}006566,gen.XM_{—}006566

FIG. 3850: PRO82267

FIG. 3851: DNA325802,NM_{—}032656,gen.NM_{—}032656

FIG. 3852: PRO82268

FIG. 3853: DNA325803,XM_{—}055013,gen.XM_{—}055013

FIG. 3854: PRO82269

FIG. 3855: DNA325804,XM_{—}113737,gen.XM_{—}113737

FIG. 3856A-C: DNA325805,XM_{—}045602,gen.XM_{—}045602

FIG. 3857: DNA325806,XM_{—}087955,gen.XM_{—}087955

FIG. 3858: PRO82272

FIG. 3859A-B: DNA325807,XM_{—}044334,gen.XM_{—}044334

FIG. 3860: PRO82273

FIG. 3861: DNA325808,XM_{—}012184,gen.XM_{—}012184

FIG. 3862: DNA325809,XM_{—}113702,gen.XM_{—}113702

FIG. 3863: PRO82275

FIG. 3864A-B: DNA270015,NM_{—}003453,gen.NM_{—}003453

FIG. 3865: PRO58410

FIG. 3866: DNA226853,NM_{—}004004,gen.NM_{—}004004

FIG. 3867: PRO37316

FIG. 3868: DNA325810,XM_{—}167911,gen.XM_{—}167911

FIG. 3869: DNA325811,XM_{—}167918,gen.XM_{—}167918

FIG. 3870: DNA325812,XM_{—}084982,gen.XM_{—}084982

FIG. 3871: PRO82278

FIG. 3872: DNA325813,NM_{—}024026,gen.NM_{—}024026

FIG. 3873: PRO82279

FIG. 3874: DNA325814,XM_{—}012638,gen.XM_{—}012638

FIG. 3875: PRO82280

FIG. 3876: DNA325815,XM_{—}167439,gen.XM_{—}167439

FIG. 3877: DNA325816,XM_{—}167906,gen.XM_{—}167906

FIG. 3878A-B: DNA325817,NM_{—}014778,gen.NM_{—}014778

FIG. 3879: PRO82283

FIG. 3880: DNA325818,XM_{—}169414,gen.XM_{—}169414

FIG. 3881A-B: DNA325819,NM_{—}006646,gen.NM_{—}006646

FIG. 3882: PRO82285

FIG. 3883: DNA325820,XM_{—}167892,gen.XM_{—}167892

FIG. 3884: DNA325821,NM_{—}015932,gen.NM_{—}015932

FIG. 3885: PRO82287

FIG. 3886: DNA325822,XM_{—}166273,gen.XM_{—}166273

FIG. 3887: DNA304669,NM_{—}002128,gen.NM_{—}002128

FIG. 3888: PRO71096

FIG. 3889: DNA325823,NM_{—}014887,gen.NM_{—}014887

FIG. 3890: PRO82289

FIG. 3891: DNA325824,NM_{—}002915,gen.NM_{—}002915

FIG. 3892: PRO82290

FIG. 3893: DNA325825,XM_{—}085017,gen.XM_{—}085017

FIG. 3894: PRO82291

FIG. 3895: DNA325826,XM_{—}017432,gen.XM_{—}017432

FIG. 3896A-B: DNA270254,NM_{—}002015,gen.NM_{—}002015

FIG. 3897: PRO58642

FIG. 3898: DNA325827,NM_{—}005830,gen.NM_{—}005830

FIG. 3899: PRO58092

FIG. 3900: DNA281436,NM_{—}003295,gen.NM_{—}003295

FIG. 3901: PRO66275

FIG. 3902: DNA325828,XM_{—}038371,gen.XM_{—}038371

FIG. 3903A-B: DNA325829,XM_{—}165636,gen.XM_{—}165636

FIG. 3904: DNA325830,XM_{—}166266,gen.XM_{—}166266

FIG. 3905: PRO82295

FIG. 3906: DNA325831,NM_{—}014166,gen.NM_{—}014166

FIG. 3907: PRO82296

FIG. 3908: DNA325832,NM_{—}021999,gen.NM_{—}021999

FIG. 3909: PRO1869

FIG. 3910: DNA325833,NM_{—}030925,gen.NM_{—}030925

FIG. 3911: PRO82297

FIG. 3912: DNA274058,NM_{—}016119,gen.NM_{—}016119

FIG. 3913: PRO61999

FIG. 3914: DNA325834,NM_{—}032565,gen.NM_{—}032565

FIG. 3915: PRO11982

FIG. 3916: DNA325835,XM_{—}085044,gen.XM_{—}085044

FIG. 3917: DNA325836,XM_{—}165639,gen.XM_{—}165639

FIG. 3918: DNA325837,XM_{—}018399,gen.XM_{—}018399

FIG. 3919: PRO82300

FIG. 3920: DNA325838,XM_{—}058977,gen.XM_{—}058977

FIG. 3921: DNA325839,XM_{—}015840,gen.XM_{—}015840

FIG. 3922: PRO82302

FIG. 3923: DNA325840,XM_{—}007199,gen.XM_{—}007199

FIG. 3924: DNA325841,XM_{—}016351,gen.XM_{—}016351

FIG. 3925: DNA325842,XM_{—}041209,gen.XM_{—}041209

FIG. 3926: DNA325843,XM_{—}058611,gen.XM_{—}058611

FIG. 3927: PRO82305

FIG. 3928: DNA325844,XM_{—}041473,gen.XM_{—}041473

FIG. 3929: PRO82306

FIG. 3930: DNA325845,XM_{—}032443,gen.XM_{—}032443

FIG. 3931: DNA325847,XM_{—}048957,gen.XM_{—}048957

FIG. 3932: DNA325848,XM_{—}015842,gen.XM_{—}015842

FIG. 3933: DNA325849,XM_{—}084997,gen.XM_{—}084997

FIG. 3934: PRO82311

FIG. 3935: DNA325850,NM_{—}024089,gen.NM_{—}024089

FIG. 3936: PRO82312

FIG. 3937A-B: DNA325851,XM_{—}049904,gen.XM_{—}049904

FIG. 3938: DNA325852,NM_{—}024537,gen.NM_{—}024537

FIG. 3939: PRO82314

FIG. 3940: DNA325853,NM_{—}023011,gen.NM_{—}023011

FIG. 3941: PRO82315

FIG. 3942: DNA325854,NM_{—}080687,gen.NM_{—}080687

FIG. 3943: PRO82316

FIG. 3944: DNA325855,XM_{—}041484,gen.XM_{—}041484

FIG. 3945: PRO82317

FIG. 3946A-B: DNA325856,XM_{—}113752,gen.XM_{—}113752

FIG. 3947: PRO82318

FIG. 3948: DNA325857,XM_{—}115215,gen.XM_{—}115215

FIG. 3949: DNA325858,XM_{—}046651,gen.XM_{—}046651

FIG. 3950: DNA325859,XM_{—}046648,gen.XM_{—}046648

FIG. 3951: DNA325860,XM_{—}046642,gen.XM_{—}046642

FIG. 3952: PRO10404

FIG. 3953: DNA325861,XM_{—}017914,gen.XM_{—}017914

FIG. 3954: PRO82321

FIG. 3955: DNA325862,XM_{—}085166,gen.XM_{—}085166

FIG. 3956: PRO82322

FIG. 3957: DNA325863,XM_{—}007316,gen.XM_{—}007316

FIG. 3958: DNA325864,XM_{—}007315,gen.XM_{—}007315

FIG. 3959: DNA325865,XM_{—}033251,gen.XM_{—}033251

FIG. 3960: DNA325866,NM_{—}024658,gen.NM_{—}024658

FIG. 3961: PRO82325

FIG. 3962: DNA210180,NM_{—}005132,gen.NM_{—}005132

FIG. 3963: PRO33717

FIG. 3964: DNA325867,XM_{—}033337,gen.XM_{—}033337

FIG. 3965: PRO82326

FIG. 3966: DNA325868,XM_{—}096772,gen.XM_{—}096772

FIG. 3967: DNA325869,XM_{—}007293,gen.XM_{—}007293

FIG. 3968: DNA325870,XM_{—}007288,gen.XM_{—}007288

FIG. 3969A-B: DNA325871,XM_{—}033391,gen.XM_{—}033391

FIG. 3970: PRO82329

FIG. 3971: DNA325872,NM_{—}017815,gen.NM_{—}017815

FIG. 3972: PRO82330

FIG. 3973: DNA325873,NM_{—}006109,gen.NM_{—}006109

FIG. 3974: PRO82331

FIG. 3975: DNA325874,XM_{—}033435,gen.XM_{—}033435

FIG. 3976: DNA225865,NM_{—}004995,gen.NM_{—}004995

FIG. 3977: PRO36328

FIG. 3978: DNA325875,XM_{—}058647,gen.XM_{—}058647

FIG. 3979: PRO82333

FIG. 3980: DNA325876,XM_{—}033445,gen.XM_{—}033445

FIG. 3981: DNA325877,NM_{—}005015,gen.NM_{—}005015

FIG. 3982: PRO82334

FIG. 3983: DNA325878,XM_{—}012377,gen.XM_{—}012377

FIG. 3984: DNA227321,NM_{—}001344,gen.NM_{—}001344

FIG. 3985: PRO37784

FIG. 3986: DNA325879,XM_{—}058646,gen.XM_{—}058646

FIG. 3987: DNA325880,XM_{—}085106,gen.XM_{—}085106

FIG. 3988: DNA325881,NM_{—}019852,gen.NM_{—}019852

FIG. 3989: PRO82338

FIG. 3990: DNA325882,XM_{—}012376,gen.XM_{—}012376

FIG. 3991: DNA325883,XM_{—}033553,gen.XM_{—}033553

FIG. 3992: DNA226105,NM_{—}002934,gen.NM_{—}002934

FIG. 3993: PRO36568

FIG. 3994: DNA325884,XM_{—}033595,gen.XM_{—}033595

FIG. 3995: PRO2871

FIG. 3996: DNA325885,XM_{—}007491,gen.XM_{—}007491

FIG. 3997: DNA325886,NM_{—}001641,gen.NM_{—}001641

FIG. 3998: PRO82342

FIG. 3999: DNA325887,NM_{—}080648,gen.NM_{—}080648

FIG. 4000: PRO82343

FIG. 4001: DNA325888,NM_{—}080649,gen.NM_{—}080649

FIG. 4002: PRO82344

FIG. 4003: DNA325889,NM_{—}017807,gen.NM_{—}017807

FIG. 4004: PRO82345

FIG. 4005A-C: DNA325890,XM_{—}4007488,gen.XM_{—}007488

FIG. 4006: DNA325891,NM_{—}021178,gen.NM_{—}021178

FIG. 4007: PRO82347

FIG. 4008: DNA325892,XM_{—}041235,gen.XM_{—}041235

FIG. 4009: PRO82348

FIG. 4010: DNA325893,NM_{—}002028,gen.NM_{—}002028

FIG. 4011: PRO82349

FIG. 4012: DNA325894,NM_{—}002083,gen.NM_{—}002083

FIG. 4013: PRO82350

FIG. 4014A-B: DNA325895,XM_{—}085127,gen.XM_{—}085127

FIG. 4015: PRO82351

FIG. 4016A-B: DNA325896,NM_{—}001530,gen.NM_{—}001530

FIG. 4017: PRO82352

FIG. 4018: DNA325897,XM_{—}058210,gen.XM_{—}058210

FIG. 4019: DNA325898,XM_{—}085141,gen.XM_{—}085141

FIG. 4020: DNA325899,NM_{—}021728,gen.NM_{—}021728

FIG. 4021: PRO82355

FIG. 4022: DNA325900,NM_{—}002306,gen.NM_{—}002306

FIG. 4023: PRO82356

FIG. 4024: DNA325901,XM_{—}007328,gen.XM_{—}007328

FIG. 4025A-B: DNA325902,XM_{—}051712,gen.XM_{—}051712

FIG. 4026: PRO82357

FIG. 4027: DNA325903,XM_{—}007324,gen.XM_{—}007324

FIG. 4028: PRO82358

FIG. 4029: DNA325904,NM_{—}002863,gen.NM_{—}002863

FIG. 4030: PRO82359

FIG. 4031: DNA325905,XM_{—}085125,gen.XM_{—}085125

FIG. 4032: DNA325906,XM_{—}031025,gen.XM_{—}031025

FIG. 4033: DNA325907,XM_{—}085066,gen.XM_{—}085066

FIG. 4034: DNA325908,XM_{—}096744,gen.XM_{—}096744

FIG. 4035: DNA325909,NM_{—}016445,gen.NM_{—}016445

FIG. 4036: PRO82364

FIG. 4037: DNA325910,NM_{—}016026,gen.NM_{—}016026

FIG. 4038: PRO82365

FIG. 4039: DNA32591 1,XM_{—}031074,gen.XM_{—}031074

FIG. 4040: DNA325912,NM_{—}001102,gen.NM_{—}001102

FIG. 4041: PRO82367

FIG. 4042: DNA225649,NM_{—}022137,gen.NM_{—}022137

FIG. 4043: PRO36112

FIG. 4044: DNA325913,XM_{—}085065,gen.XM_{—}085065

FIG. 4045: DNA325914,XM_{—}007441,gen.XM_{—}007441

FIG. 4046: DNA325915,NM_{—}006821,gen.NM_{—}006821

FIG. 4047: PRO82369

FIG. 4048: DNA325916,NM_{—}006432,gen.NM_{—}006432

FIG. 4049: PRO2066

FIG. 4050A-B: DNA325917,XM_{—}085151,gen.XM_{—}085151

FIG. 4051: PRO82370

FIG. 4052: DNA325918,NM_{—}002632,gen.NM_{—}002632

FIG. 4053: PRO82371

FIG. 4054: DNA325919,XM_{—}085162,gen.XM_{—}085162

FIG. 4055: DNA325920,NM_{—}012111,gen.NM_{—}012111

FIG. 4056: PRO82373

FIG. 4057: DNA325921,NM_{—}024824,gen.NM_{—}024824

FIG. 4058: PRO82374

FIG. 4059: DNA269498,NM_{—}002802,gen.NM_{—}002802

FIG. 4060: PRO57917

FIG. 4061: DNA325922,XM_{—}058677,gen.XM_{—}058677

FIG. 4062: PRO82375

FIG. 4063: DNA325923,NM_{—}006888,gen.NM_{—}006888

FIG. 4064: PRO4904

FIG. 4065: DNA325924,NM_{—}001275,gen.NM_{—}001275

FIG. 4066: PRO2054

FIG. 4067: DNA325925,XM_{—}029288,gen.XM_{—}029288

FIG. 4068A-B: DNA325926,XM_{—}016487,gen.XM_{—}016487

FIG. 4069: DNA325927,NM_{—}020414,gen.NM_{—}020414

FIG. 4070: PRO62099

FIG. 4071: DNA325928,XM_{—}016486,gen.XM_{—}016486

FIG. 4072: DNA325929,XM_{—}007483,gen.XM_{—}007483

FIG. 4073: DNA325930,XM_{—}028358,gen.XM_{—}028358

FIG. 4074: DNA325931,XM_{—}028347,gen.XM_{—}028347

FIG. 4075: DNA325932,XM_{—}028322,gen.XM_{—}028322

FIG. 4076: PRO82381

FIG. 4077: DNA325933,XM_{—}056317,gen.XM_{—}056317

FIG. 4078: PRO82382

FIG. 4079: DNA151893,NM_{—}021966,gen.NM_{—}021966

FIG. 4080: PRO12916

FIG. 4081: DNA325934,XM_{—}007272,gen.XM_{—}007272

FIG. 4082: DNA325935,XM_{—}090914,gen.XM_{—}090914

FIG. 4083: PRO82383

FIG. 4084: DNA325936,NM_{—}022747,gen.NM_{—}022747

FIG. 4085: PRO82384

FIG. 4086: DNA325937,XM_{—}041014,gen.XM_{—}041014

FIG. 4087: PRO60575

FIG. 4088: DNA325938,NM_{—}003836,gen.NM_{—}003836

FIG. 4089: PRO82385

FIG. 4090A-B: DNA325939,XM_{—}040952,gen.XM_{—}040952

FIG. 4091: DNA325940,XM_{—}058618,gen.XM_{—}058618

FIG. 4092: DNA325941,NM_{—}005348,gen.NM_{—}005348

FIG. 4093: PRO82388

FIG. 4094: DNA325942,XM_{—}040942,gen.XM_{—}040942

FIG. 4095: DNA226324,NM_{—}014226,gen.NM_{—}014226

FIG. 4096: PRO36787

FIG. 4097A-B: DNA325943,XM_{—}007254,gen.XM_{—}007254

FIG. 4098A-B: DNA325944,NM_{—}01969,gen.NM_{—}001969

FIG. 4099: PRO82391

FIG. 4100: DNA325945,XM_{—}040898,gen.XM_{—}040898

FIG. 4101: DNA325946,NM_{—}005432,gen.NM_{—}005432

FIG. 4102: PRO60070

FIG. 4103A-B: DNA325947,XM_{—}050278,gen.XM_{—}050278

FIG. 4104: PRO82393

FIG. 4105: DNA325948,XM_{—}113759,gen.XM_{—}113759

FIG. 4106: DNA325949,NM_{—}006427,gen.NM_{—}006427

FIG. 4107: PRO82395

FIG. 4108: DNA325950,NM_{—}021709,gen.NM_{—}021709

FIG. 4109: PRO82396

FIG. 4110: DNA103509,NM_{—}005163,gen.NM_{—}005163

FIG. 4111: PRO4836

FIG. 4112: DNA325951,NM_{—}017955,gen.NM_{—}017955

FIG. 4113: PRO82397

FIG. 4114: DNA325952,XM_{—}088588,gen.XM_{—}088588

FIG. 4115: DNA325953,XM_{—}060012,gen.XM_{—}060012

FIG. 4116: DNA325954,XM_{—}034953,gen.XM_{—}034953

FIG. 4117: PRO82400

FIG. 4118: DNA325955,XM_{—}058636,gen.XM_{—}058636

FIG. 4119: DNA325956,XM_{—}035014,gen.XM_{—}035014

FIG. 4120: DNA325957,XM_{—}088587,gen.XM_{—}088587

FIG. 4121: DNA325958,XM_{—}088589,gen.XM_{—}088589

FIG. 4122: DNA325959,XM_{—}071801,gen.XM_{—}071801

FIG. 4123: DNA325960,XM_{—}018054,gen.XM_{—}018054

FIG. 4124: DNA325961,XM_{—}091108,gen.XM_{—}091108

FIG. 4125A-B: DNA325962,XM_{—}039225,gen.XM_{—}039225

FIG. 4126: PRO82408

FIG. 4127: DNA325963,XM_{—}165921,gen.XM_{—}165921

FIG. 4128: PRO82409

FIG. 4129: DNA325964,XM_{—}007751,gen.XM_{—}007751

FIG. 4130: DNA325965,XM_{—}085203,gen.XM_{—}085203

FIG. 4131: PRO82411

FIG. 4132: DNA325966,XM_{—}085204,gen.XM_{—}085204

FIG. 4133: DNA325967,XM_{—}012398,gen.XM_{—}012398

FIG. 4134A-B: DNA325968,XM_{—}036727,gen.XM_{—}036727

FIG. 4135: DNA325969,XM_{—}017240,gen.XM_{—}017240

FIG. 4136: DNA325970,NM_{—}020149,gen.NM_{—}020149

FIG. 4137: PRO82415

FIG. 4138A-B: DNA325971,XM_{—}031617,gen.XM_{—}031617

FIG. 4139A-B: DNA325972,NM_{—}001211,gen.NM_{—}001211

FIG. 4140: PRO82417

FIG. 4141A-B: DNA151831,NM_{—}004573,gen.NM_{—}004573

FIG. 4142: PRO12198

FIG. 4143: DNA325973,NM_{—}130468,gen.NM_{—}130468

FIG. 4144: PRO82418

FIG. 4145: DNA325974,XM_{—}031554,gen.XM_{—}031554

FIG. 4146: PRO82419

FIG. 4147: DNA325975,XM_{—}031515,gen.XM_{—}031515

FIG. 4148: DNA325976,NM_{—}024111,gen.NM_{—}024111

FIG. 4149: PRO82421

FIG. 4150: DNA325977,NM_{—}032196,gen.NM_{—}032196

FIG. 4151: PRO82422

FIG. 4152: DNA325978,NM_{—}016359,gen.NM_{—}016359

FIG. 4153: PRO82423

FIG. 4154: DNA325979,NM_{—}018454,gen.NM_{—}018454

FIG. 4155: PRO82424

FIG. 4156A-B: DNA325980,XM_{—}007545,gen.XM_{—}007545

FIG. 4157: DNA325981,XM_{—}091159,gen.XM_{—}091159

FIG. 4158: PRO82425

FIG. 4159: DNA325982,XM_{—}031718,gen.XM_{—}031718

FIG. 4160: DNA325983,XM_{—}085307,gen.XM_{—}085307

FIG. 4161: DNA227559,NM_{—}000070,gen.NM_{—}000070

FIG. 4162: PRO38022

FIG. 4163A-B: DNA325984,XM_{—}113823,gen.XM_{—}113823

FIG. 4164: PRO82428

FIG. 4165: DNA325985,XM_{—}016713,gen.XM_{—}016713

FIG. 4166: PRO82429

FIG. 4167A-B: DNA325986,XM_{—}007531,gen.XM_{—}007531

FIG. 4168: DNA325987,NM_{—}014444,gen.NM_{—}014444

FIG. 4169: PRO82431

FIG. 4170A-B: DNA227206,NM_{—}005657,gen.NM_{—}005657

FIG. 4171: PRO37669

FIG. 4172: DNA325988,NM_{—}020990,gen.NM_{—}020990

FIG. 4173: PRO82432

FIG. 4174: DNA325989,NM_{—}005313,gen.NM_{—}005313

FIG. 4175: PRO2732

FIG. 4176: DNA325990,NM_{—}005770,gen.NM_{—}005770

FIG. 4177: PRO82433

FIG. 4178: DNA325991,NM_{—}004048,gen.NM_{—}004048

FIG. 4179: PRO4379

FIG. 4180: DNA325992,XM_{—}032403,gen.XM_{—}032403

FIG. 4181: PRO82434

FIG. 4182: DNA219233,NM_{—}014335,gen.NM_{—}014335

FIG. 4183: PRO34557

FIG. 4184A-C: DNA325993,XM_{—}034890,gen.XM_{—}034890

FIG. 4185: PRO82435

FIG. 4186: DNA325994,XM_{—}058684,gen.XM_{—}058684

FIG. 4187: DNA325995,NM_{—}003104,gen.NM_{—}003104

FIG. 4188: PRO82437

FIG. 4189: DNA325996,XM_{—}007651,gen.XM_{—}007651

FIG. 4190: PRO82438

FIG. 4191: DNA325997,XM_{—}090991,gen.XM_{—}090991

FIG. 4192: PRO82439

FIG. 4193: DNA325998,NM_{—}016304,gen.NM_{—}016304

FIG. 4194: PRO82440

FIG. 4195: DNA325999,NM_{—}017610,gen.NM_{—}017610

FIG. 4196: PRO82441

FIG. 4197: DNA326000,NM_{—}004701,gen.NM_{—}004701

FIG. 4198: PRO82442

FIG. 4199A-B: DNA326001,XM_{—}012418,gen.XM_{—}012418

FIG. 4200: DNA326002,XM_{—}039702,gen.XM_{—}039702

FIG. 4201: PRO82444

FIG. 4202: DNA326003,3XM_{—}1 13266,gen.XM_{—}113266

FIG. 4203: DNA326004,NM_{—}001218,gen.NM_{—}01218

FIG. 4204: PRO54594

FIG. 4205: DNA326005,NM_{—}015920,gen.NM_{—}015920

FIG. 4206: PRO82446

FIG. 4207: DNA326006,XM_{—}1 13268,gen.XM_{—}113268

FIG. 4208: DNA255340,NM_{—}017684,gen.NM_{—}017684

FIG. 4209: PRO50409

FIG. 4210: DNA326007,NM_{—}002537,gen.NM_{—}002537

FIG. 4211: DNA326008,XM_{—}085283,gen.XM_{—}085283

FIG. 4212: PRO82448

FIG. 4213: DNA326009,XM_{—}016985,gen.XM_{—}016985

FIG. 4214: DNA234442,NM_{—}014736,gen.NM_{—}014736

FIG. 4215: PRO38852

FIG. 4216: DNA326010,NM_{—}022048,gen.NM_{—}022048

FIG. 4217: PRO82450

FIG. 4218: DNA326011,NM_{—}000942,gen.NM_{—}000942

FIG. 4219: PRO2720

FIG. 4220: DNA326012,XM_{—}050964,gen.XM_{—}050964

FIG. 4221: DNA326013,XM_{—}007623,gen.XM_{—}007623

FIG. 4222A-B: DNA326014,NM_{—}133375,gen.NM_{—}133375

FIG. 4223: PRO82453

FIG. 4224: DNA226646,NM_{—}017882,gen.NM_{—}017882

FIG. 4225: PRO37109

FIG. 4226: DNA326015,NM_{—}015322,gen.NM_{—}015322

FIG. 4227: PRO82454

FIG. 4228: DNA326016,NM_{—}001003,gen.NM_{—}001003

FIG. 4229: PRO82455

FIG. 4230A-B: DNA326017,XM_{—}051463,gen.XM_{—}051463

FIG. 4231: PRO82456

FIG. 4232: DNA326018,NM_{—}018357,gen.NM_{—}018357

FIG. 4233: PRO82457

FIG. 4234: DNA326019,XM_{—}063639,gen.XM_{—}063639

FIG. 4235: PRO82458

FIG. 4236: DNA326020,XM_{—}085249,gen.XM_{—}085249

FIG. 4237: DNA326021,XM_{—}016076,gen.XM_{—}016076

FIG. 4238: PRO82460

FIG. 4239: DNA326022,XM_{—}015366,gen.XM_{—}015366

FIG. 4240: PRO82461

FIG. 4241: DNA326023,XM_{—}096060,gen.XM_{—}096060

FIG. 4242: DNA287331,NM_{—}002654,gen.NM_{—}002654

FIG. 4243: PRO69595

FIG. 4244: DNA326024,XM_{—}037778,gen.XM_{—}037778

FIG. 4245: DNA326025,XM_{—}096842,gen.XM_{—}096842

FIG. 4246: DNA326026,NM_{—}022369,gen.NM_{—}022369

FIG. 4247: PRO82465

FIG. 4248: DNA326027,NM_{—}032907,gen.NM_{—}032907

FIG. 4249: PRO82466

FIG. 4250: DNA326028,XM_{—}058699,gen.XM_{—}058699

FIG. 4251: DNA326029,XM_{—}118637,gen.XM_{—}118637

FIG. 4252: DNA326030,XM_{—}053585,gen.XM_{—}053585

FIG. 4253: PRO82469

FIG. 4254: DNA326031,XM_{—}085239,gen.XM_{—}085239

FIG. 4255: PRO82470

FIG. 4256: DNA326032,XM_{—}034897,gen.XM_{—}034897

FIG. 4257A-B: DNA326033,XM_{—}057020,gen.XM_{—}057020

FIG. 4258: PRO82472

FIG. 4259: DNA326034,NM_{—}000743,gen.NM_{—}000743

FIG. 4260: PRO61219

FIG. 4261: DNA326035,NM_{—}002789,gen.NM_{—}002789

FIG. 4262: PRO60499

FIG. 4263: DNA326036,XM_{—}091100,gen.XM_{—}091100

FIG. 4264: PRO82473

FIG. 4265: DNA255370,NM_{—}012170,gen.NM_{—}012170

FIG. 4266: PRO50438

FIG. 4267: DNA273014,NM_{—}000126,gen.NM_{—}000126

FIG. 4268: PRO61085

FIG. 4269: DNA326037,XM_{—}044565,gen.XM_{—}044565

FIG. 4270: DNA326038,NM_{—}025234,gen.NM_{—}025234

FIG. 4271: PRO82475

FIG. 4272: DNA326039,XM_{—}044569,gen.XM_{—}044569

FIG. 4273: DNA326040,NM_{—}005724,gen.NM_{—}005724

FIG. 4274: PRO730

FIG. 4275: DNA326041,XM_{—}049354,gen.XM_{—}049354

FIG. 4276: PRO82477

FIG. 4277: DNA326042,NM_{—}007364,gen.NM_{—}007364

FIG. 4278: DNA326043,XM_{—}044593,gen.XM_{—}044593

FIG. 4279: DNA326044,NM_{—}006791,gen.NM_{—}006791

FIG. 4280: PRO82479

FIG. 4281: DNA326045,XM_{—}060042,gen.XM_{—}060042

FIG. 4282: DNA326046,XM_{—}085215,gen.XM_{—}085215

FIG. 4283: DNA326047,NM_{—}001021,gen.NM_{—}001021

FIG. 4284: PRO82482

FIG. 4285: DNA326048,XM_{—}031404,gen.XM_{—}031404

FIG. 4286: DNA326049,XM_{—}096844,gen.XM_{—}096844

FIG. 4287: DNA326050,XM_{—}045681,gen.XM_{—}045681

FIG. 4288: PRO82485

FIG. 4289: DNA326051,XM_{—}085280,gen.XM_{—}085280

FIG. 4290: DNA326052,NM_{—}022839,gen.NM_{—}022839

FIG. 4291: PRO82487

FIG. 4292: DNA326053,XM_{—}031354,gen.XM_{—}031354

FIG. 4293: DNA326054,NM_{—}002168,gen.NM_{—}002168

FIG. 4294: PRO82489

FIG. 4295: DNA326055,XM_{—}031292,gen.XM_{—}031292

FIG. 4296: DNA326056,NM_{—}022566,gen.NM_{—}22566

FIG. 4297: PRO82491

FIG. 4298A-B: DNA326057,XM_{—}051860,gen.XM_{—}051860

FIG. 4299: PRO82492

FIG. 4300: DNA275144,NM_{—}000137,gen.NM_{—}000137

FIG. 4301: PRO62852

FIG. 4302: DNA326058,NM_{—}016645,gen.NM_{—}016645

FIG. 4303: PRO82493

FIG. 4304: DNA326059,XM_{—}044523,gen.XM_{—}044523

FIG. 4305: DNA150485,NM_{—}006384,gen.NM_{—}006384

FIG. 4306: PRO12774

FIG. 4307A-B: DNA326060,XM_{—}044533,gen.XM_{—}044533

FIG. 4308: PRO82495

FIG. 4309A-C: DNA326061,XM_{—}054900,gen.XM_{—}054900

FIG. 4310: DNA326062,NM_{—}032162,gen.NM_{—}032162

FIG. 4311A-B: DNA326063,XM_{—}015835,gen.XM_{—}015835

FIG. 4312: DNA326064,NM_{—}018668,gen.NM_{—}018668

FIG. 4313: PRO82499

FIG. 4314: DNA326065,XM_{—}085262,gen.XM_{—}085262

FIG. 4315: DNA326066,NM_{—}033544,gen.NM_{—}033544

FIG. 4316: PRO82501

FIG. 4317: DNA326067,XM_{—}049372,gen.XM_{—}049372

FIG. 4318: PRO82502

FIG. 4319: DNA326068,XM_{—}017971,gen.XM_{—}017971

FIG. 4320: DNA275181,NM_{—}003090,gen.NM_{—}003090

FIG. 4321: PRO62882

FIG. 4322: DNA326069,XM_{—}012462,gen.XM_{—}012462

FIG. 4323A-B: DNA326070,XM_{—}085525,gen.XM_{—}085525

FIG. 4324: PRO82505

FIG. 4325: DNA326071,XM_{—}165923,gen.XM_{—}165923

FIG. 4326: DNA326072,XM_{—}113836,gen.XM_{—}113836

FIG. 4327: DNA326073,NM_{—}017668,gen.NM_{—}017668

FIG. 4328: PRO82508

FIG. 4329: DNA326074,XM_{—}027309,gen.XM_{—}027309

FIG. 4330: PRO82509

FIG. 4331: DNA326075,XM_{—}018432,gen.XM_{—}018432

FIG. 4332: PRO82510

FIG. 4333: DNA326076,XM_{—}115352,gen.XM_{—}115352

FIG. 4334: DNA326077,XM_{—}027365,gen.XM_{—}027365

FIG. 4335: DNA326078,NM_{—}016641,gen.NM_{—}016641

FIG. 4336: PRO38464

FIG. 4337: DNA326079,XM_{—}058796,gen.XM_{—}058796

FIG. 4338: DNA326080,XM_{—}017984,gen.XM_{—}017984

FIG. 4339: PRO82513

FIG. 4340: DNA326081,NM_{—}020677,gen.NM_{—}020677

FIG. 4341: PRO82514

FIG. 4342: DNA326082,XM_{—}036680,gen.XM_{—}036680

FIG. 4343: PRO37961

FIG. 4344A-B: DNA326083,XM_{—}048119,gen.XM_{—}048119

FIG. 4345: PRO82515

FIG. 4346: DNA326084,NM_{—}024589,gen.NM_{—}024589

FIG. 4347: PRO82516

FIG. 4348: DNA326085,XM_{—}050534,gen.XM_{—}050534

FIG. 4349: PRO82517

FIG. 4350: DNA326086,NM_{—}024571,gen.NM_{—}024571

FIG. 4351: PRO82518

FIG. 4352: DNA326087,XM_{—}027558,gen.XM_{—}027558

FIG. 4353: DNA326088,XM_{—}008126,gen.XM_{—}008126

FIG. 4354: DNA326089,NM_{—}000517,gen.NM_{—}000517

FIG. 4355: PRO3629

FIG. 4356: DNA326090,NM_{—}000558,gen.NM_{—}000558

FIG. 4355: PRO3629

FIG. 4356: DNA326090,NM_{—}000558,gen.NM_{—}000558

FIG. 4357: PRO3629

FIG. 4358: DNA326091,NM_{—}018032,gen.NM_{—}018032

FIG. 4359: PRO38311

FIG. 4360: DNA273839,NM_{—}006428,gen.NM_{—}006428

FIG. 4361: PRO61799

FIG. 4362A-B: DNA256844,NM_{—}005632,gen.NM_{—}005632

FIG. 4363: PRO51775

FIG. 4364: DNA326092,XM_{—}083939,gen.XM_{—}083939

FIG. 4365: PRO82521

FIG. 4366: DNA326093,NM_{—}058192,gen.NM_{—}058192

FIG. 4367: PRO82522

FIG. 4368: DNA326094,XM_{—}027412,gen.XM_{—}027412

FIG. 4369: PRO82523

FIG. 4370: DNA256886,NM_{—}014587,gen.NM_{—}014587

FIG. 4371: PRO51815

FIG. 4372A-B: DNA326095,NM_{—}001287,gen.NM_{—}001287

FIG. 4373: PRO38480

FIG. 4374: DNA254781,NM_{—}016111,gen.NM_{—}016111

FIG. 4375: PRO49879

FIG. 4376: DNA326096,XM_{—}034586,gen.XM_{—}034586

FIG. 4377: PRO82524

FIG. 4378: DNA326097,NM_{—}023936,gen.NM_{—}023936

FIG. 4379: PRO82525

FIG. 4380: DNA326098,XM_{—}034590,gen.XM_{—}034590

FIG. 4381: PRO82526

FIG. 4382: DNA326099,NM_{—}002952,gen.NM_{—}002952

FIG. 4383: PRO82527

FIG. 4384: DNA326100,NM_{—}006453,gen.NM_{—}006453

FIG. 4385: PRO82528

FIG. 4386: DNA326101,NM_{—}014353,gen.NM_{—}014353

FIG. 4387: PRO82529

FIG. 4388: DNA326102,NM_{—}032271,gen.NM_{—}032271

FIG. 4389: PRO82530

FIG. 4390: DNA326103,XM_{—}028848,gen.XM_{—}028848

FIG. 4391: PRO82531

FIG. 4392: DNA326104,NM_{—}006711,gen.NM_{—}006711

FIG. 4393: PRO82532

FIG. 4394: DNA326105,NM_{—}080594,gen.NM_{—}080594

FIG. 4395: PRO82533

FIG. 4396: DNA326106,NM_{—}024339,gen.NM_{—}024339

FIG. 4397: PRO82534

FIG. 4398: DNA326107,NM_{—}016639,gen.NM_{—}016639

FIG. 4399: PRO12683

FIG. 4400: DNA326108,NM_{—}021195,gen.NM_{—}021195

FIG. 4401: PRO82535

FIG. 4402: DNA326109,NM_{—}004203,gen.NM_{—}004203

FIG. 4403: PRO82536

FIG. 4404: DNA3261 10,XM_{—}058784,gen.XM_{—}058784

FIG. 4405: PRO82537

FIG. 4406: DNA326111,NM_{—}024507,gen.NM_{—}024507

FIG. 4407: PRO82538

FIG. 4408: DNA326112,NM_{—}006799,gen.NM_{—}006799

FIG. 4409: PRO303

FIG. 4410A-C: DNA326113,XM_{—}036528,gen.XM_{—}036528

FIG. 4411: DNA326114,NM_{—}025108,gen.NM_{—}025108

FIG. 4412: PRO82540

FIG. 4413A-C: DNA326115,XM_{—}165411,gen.XM_{—}165411

FIG. 4414: DNA326116,NM_{—}016292,gen.NM_{—}016292

FIG. 4415: PRO82542

FIG. 4416: DNA326117,NM_{—}002484,gen.NM_{—}002484

FIG. 4417: PRO82543

FIG. 4418: DNA326118,XM_{—}113845,gen.XM_{—}113845

FIG. 4419: PRO82544

FIG. 4420: DNA326119,XM_{—}113843,gen.XM_{—}113843

FIG. 4421: DNA97293,NM_{—}003366,gen.NM_{—}003366

FIG. 4422: PRO3640

FIG. 4423: DNA326120,NM_{—}006110,gen.NM_{—}006110

FIG. 4424: PRO82546

FIG. 4425: DNA326121,XM_{—}085445,gen.XM_{—}085445

FIG. 4426: DNA326122,XM_{—}113876,gen.XM_{—}113876

FIG. 4427A-B: DNA326123,XM_{—}055195,gen.XM_{—}055195

FIG. 4428: PRO82548

FIG. 4429: DNA326124,XM_{—}113291,gen.XM_{—}113291

FIG. 4430A-B: DNA326125,XM_{—}007988,gen.XM_{—}007988

FIG. 4431: DNA326126,XM_{—}113874,gen.XM_{—}113874

FIG. 4432: DNA326127,XM_{—}102377,gen.XM_{—}102377

FIG. 4433: PRO82551

FIG. 4434: DNA326128,XM_{—}086278,gen.XM_{—}086278

FIG. 4435: DNA326129,XM_{—}085452,gen.XM_{—}085452

FIG. 4436: DNA326130,NM_{—}018054,gen.NM_{—}018054

FIG. 4437: PRO82554

FIG. 4438A-B: DNA326131,XM_{—}056260,gen.XM_{—}056260

FIG. 4439: PRO82555

FIG. 4440: DNA326132,NM_{—}032626,gen.NM_{—}032626

FIG. 4441: PRO82556

FIG. 4442: DNA326133,NM_{—}005030,gen.NM_{—}005030

FIG. 4443: PRO82557

FIG. 4444: DNA326134,NM_{—}032486,gen.NM_{—}032486

FIG. 4445: PRO82558

FIG. 4446: DNA289522,NM_{—}005003,gen.NM_{—}005003

FIG. 4447: PRO70276

FIG. 4448: DNA326135,XM_{—}085340,gen.XM_{—}085340

FIG. 4449: DNA326136,NM_{—}003752,gen.NM_{—}003752

FIG. 4450: PRO60325

FIG. 4451: DNA326137,NM_{—}012248,gen.NM_{—}012248

FIG. 4452: PRO82560

FIG. 4453A-B: DNA326138,XM_{—}046035,gen.XM_{—}046035

FIG. 4454: DNA326139,NM_{—}024671,gen.NM_{—}024671

FIG. 4455: PRO82562

FIG. 4456: DNA326140,NM_{—}033410,gen.NM_{—}033410

FIG. 4457: PRO82563

FIG. 4458: DNA326141,NM_{—}024031,gen.NM_{—}024031

FIG. 4459: PRO82564

FIG. 4460A-B: DNA326142,XM_{—}034375,gen.XM_{—}034375

FIG. 4461: DNA326143,XM_{—}012569,gen.XM_{—}012569

FIG. 4462: DNA326144,XM_{—}050194,gen.XM_{—}050194

FIG. 4463: DNA326145,XM_{—}008106,gen.XM_{—}008106

FIG. 4464: PRO82567

FIG. 4465: DNA326146,NM_{—}004960,gen.NM_{—}004960

FIG. 4466: PRO82568

FIG. 4467: DNA326147,XM_{—}113293,gen.XM_{—}113293

FIG. 4468: DNA326148,NM_{—}022744,gen.NM_{—}022744

FIG. 4469: PRO82570

FIG. 4470: DNA326149,NM_{—}024048,gen.NM_{—}024048

FIG. 4471: PRO82571

FIG. 4472: DNA326150,XM_{—}018088,gen.XM_{—}018088

FIG. 4473: PRO82572

FIG. 4474: DNA326151,XM_{—}007963,gen.XM_{—}007963

FIG. 4475: PRO82573

FIG. 4476: DNA274002,NM_{—}014321,gen.NM_{—}014321

FIG. 4477: PRO61948

FIG. 4478: DNA326152,XM_{—}015700,gen.XM_{—}015700

FIG. 4479: DNA326153,XM_{—}051219,gen.XM_{—}051219

FIG. 4480: DNA326154,XM_{—}085393,gen.XM_{—}085393

FIG. 4481: PRO82576

FIG. 4482: DNA326155,XM_{—}085395,gen.XM_{—}085395

FIG. 4483: DNA326156,XM_{—}091270,gen.XM_{—}091270

FIG. 4484: DNA326157,XM_{—}165656,gen.XM_{—}165656

FIG. 4485: DNA326158,NM_{—}032330,gen.NM_{—}032330

FIG. 4486: PRO82579

FIG. 4487: DNA254532,NM_{—}001043,gen.NM_{—}001043

FIG. 4488: PRO49639

FIG. 4489: DNA326159,XM_{—}165658,gen.XM_{—}085434

FIG. 4490: DNA326160,XM_{—l}66285,gen.XM_{—}166285

FIG. 4491: DNA326161,XM_{—}166282,gen.XM_{—}166282

FIG. 4492: PRO82582

FIG. 4493: DNA326162,XM_{—}165657,gen.XM_{—}165657

FIG. 4494: PRO82583

FIG. 4495: DNA326163,NM_{—}032038,gen.NM_{—}032038

FIG. 4496: PRO82584

FIG. 4497: DNA326164,XM_{—}008065,gen.XM_{—}008065

FIG. 4498: DNA326165,NM_{—}017458,gen.NM_{—}017458

FIG. 4499: PRO82585

FIG. 4500: DNA326166,NM_{—}005115,gen.NM_{—}005115

FIG. 4501: PRO82586

FIG. 4502: DNA326167,NM_{—}024516,gen.NM_{—}024516

FIG. 4503: PRO82587

FIG. 4504: DNA326168,XM_{—}113299,gen.XM_{—}113299

FIG. 4505: DNA326169,XM_{—}055771,gen.XM_{—}055771

FIG. 4506: PRO82589

FIG. 4507: DNA271171,NM_{—}007317,gen.NM_{—}007317

FIG. 4508: PRO59491

FIG. 4509: DNA326170,XM_{—}008064,gen.XM_{—}008064

FIG. 4510: PRO82590

FIG. 4511: DNA326171,NM_{—}003123,gen.NM_{—}003123

FIG. 4512: PRO2355

FIG. 4513: DNA326172,XM_{—}085442,gen.XM_{—}085442

FIG. 4514: DNA326173,XM_{—}055132,gen.XM_{—}055132

FIG. 4515: PRO82592

FIG. 4516: DNA274180,NM_{—}007074,gen.NM_{—}107074

FIG. 4517: PRO62110

FIG. 4518: DNA326174,NM_{—}002720,gen.NM_{—}002720

FIG. 4519: PRO42208

FIG. 4520: DNA287355,NM_{—}000034,gen.NM_{—}000034

FIG. 4521: PRO69617

FIG. 4523: PRO82593

FIG. 4524: DNA326176,XM_{—}085434,gen.XM_{—}085434

FIG. 4525: PRO82594

FIG. 4526: DNA326177,XM_{—}058116,gen.XM_{—}058116

FIG. 4527: DNA326178,XM_{—}165649,gen.XM_{—}165649

FIG. 4528: DNA326179,XM_{—}165647,gen.XM_{—}165647

FIG. 4529: PRO82597

FIG. 4530: DNA194805,NM_{—}014685,gen.NM_{—}014685

FIG. 4531: PRO24075

FIG. 4532: DNA326180,XM_{—}166277,gen.XM_{—}166277

FIG. 4533: PRO82598

FIG. 4534: DNA326181,XM_{—}165645,gen.XM_{—}165645

FIG. 4535: DNA326182,NM_{—}018110,gen.NM_{—}018110

FIG. 4536: PRO82599

FIG. 4537: DNA326183,XM_{—}165648,gen.XM_{—}165648

FIG. 4538: DNA326184,XM_{—}167453,gen.XM_{—}167453

FIG. 4539: DNA326185,NM_{—}022770,gen.NM_{—}022770

FIG. 4540: PRO82602

FIG. 4541: DNA326186,XM_{—}167456,gen.XM_{—}167456

FIG. 4542: PRO82603

FIG. 4543: DNA326187,XM_{—}058745,gen.XM_{—}058745

FIG. 4544: DNA326188,XM_{—}091420,gen.XM_{—}091420

FIG. 4545: DNA326189,NM_{—}004691,gen.NM_{—}004691

FIG. 4546: PRO82606

FIG. 4547: DNA326190,NM_{—}000196,gen.NM_{—}000196

FIG. 4548: PRO82607

FIG. 4549A-B: DNA326191,NM_{—}004360,gen.NM_{—}004360

FIG. 4550: PRO2672

FIG. 4551: DNA326192,XM_{—}039306,gen.XM_{—}039306

FIG. 4552: PRO82608

FIG. 4553: DNA326193,NM_{—}030579,gen.NM_{—}030579

FIG. 4554: PRO82609

FIG. 4555: DNA326194,XM_{—}012487,gen.XM_{—}012487

FIG. 4556: DNA326195,NM_{—}014062,gen.NM_{—}014062

FIG. 4557: PRO82611

FIG. 4558: DNA326196,XM_{—}085471,gen.XM_{—}085471

FIG. 4559: PRO82612

FIG. 4560: DNA326197,XM_{—}113855,gen.XM_{—}113855

FIG. 4561: DNA326198,XM_{—}085475,gen.XM_{—}085475

FIG. 4562: DNA326199,XM_{—}028151,gen.XM_{—}028151

FIG. 4563: PRO82615

FIG. 4564: DNA275408,NM_{—}001605,gen.NM_{—}001605

FIG. 4565: PRO63068

FIG. 4566: DNA326200,NM_{—}007242,gen.NM_{—}007242

FIG. 4567: PRO82616

FIG. 4568: DNA189703,NM_{—}005548,gen.NM_{—}005548

FIG. 4569: PRO22637

FIG. 4570: DNA326201,XM_{—}113853,gen.XM_{—}113853

FIG. 4571: DNA326202,NM_{—}032140,gen.NM_{—}032140

FIG. 4572: PRO82618

FIG. 4573: DNA326203,NM_{—}030819,gen.NM_{—}030819

FIG. 4574: PRO82619

FIG. 4575: DNA304704,NM_{—}005796,gen.NM_{—}005796

FIG. 4576: PRO71130

FIG. 4577: DNA326204,XM_{—}043047,gen.XM_{—}043047

FIG. 4578: PRO49967

FIG. 4579: DNA88261,NM_{—}001907,gen.NM_{—}001907

FIG. 4580: PRO2719

FIG. 4581A-B: DNA326205,NM_{—}005072,gen.NM_{—}005072

FIG. 4582: PRO4814

FIG. 4583: DNA326206,XM_{—}165410,gen.XM_{—}165410

FIG. 4584: DNA326207,NM_{—}017803,gen.NM_{—}017803

FIG. 4585: PRO82621

FIG. 4586A-B: DNA326208,NM_{—}004555,gen.NM_{—}004555

FIG. 4587: PRO82622

FIG. 4588A-B: DNA326209,NM_{—}018124,gen.NM_{—}018124

FIG. 4589: PRO82623

FIG. 4590: DNA326210,XM_{—}091399,gen.XM_{—}091399

FIG. 4591: PRO82624

FIG. 4592A-B: DNA326211,NM_{—}014003,gen.NM_{—}014003

FIG. 4593: PRO82625

FIG. 4594: DNA326212,NM_{—}017853,gen.NM_{—}017853

FIG. 4595: PRO82626

FIG. 4596: DNA326213,XM_{—}042621,gen.XM_{—}042621

FIG. 4597: DNA326214,XM_{—}064091,gen.XM_{—}064091

FIG. 4598: PRO82627

FIG. 4599: DNA326215,XM_{—}085981,gen.XM_{—}085981

FIG. 4600A-B: DNA326216,XM_{—}051778,gen.XM_{—}051778

FIG. 4601: PRO82629

FIG. 4602: DNA326217,NM_{—}004483,gen.NM_{—}004483

FIG. 4603: PRO82630

FIG. 4604: DNA326218,NM_{—}020188,gen.NM_{—}020188

FIG. 4605: PRO82631

FIG. 4606: DNA326219,XM_{—}033922,gen.XM_{—}033922

FIG. 4607: PRO82632

FIG. 4608: DNA326220,XM_{—}113840,gen.XM_{—}113840

FIG. 4609: PRO82633

FIG. 4610: DNA326221,NM_{—}016095,gen.NM_{—}016095

FIG. 4611: PRO82634

FIG. 4612: DNA326222,NM_{—}006067,gen.NM_{—}006067

FIG. 4613: PRO50658

FIG. 4614: DNA326223,NM_{—}001861,gen.NM_{—}001861

FIG. 4615: PRO82635

FIG. 4616A-B: DNA326224,XM_{—}085483,gen.XM_{—}085483

FIG. 4617: DNA326225,NM_{—}017566,gen.NM_{—}017566

FIG. 4618: PRO82637

FIG. 4619: DNA326226,XM_{—}057150,gen.XM_{—}057150

FIG. 4620: PRO82638

FIG. 4621: DNA326227,XM_{—}058739,gen.XM_{—}058739

FIG. 4622: DNA326228,XM_{—}085327,gen.XM_{—}085327

FIG. 4623: PRO82640

FIG. 4624: DNA326229,XM_{—}047436,gen.XM_{—}047436

FIG. 4625: PRO82641

FIG. 4626: DNA227234,NM_{—}002386,gen.NM_{—}002386

FIG. 4627: PRO37697

FIG. 4628: DNA326230,NM_{—}014972,gen.NM_{—}014972

FIG. 4629: PRO82642

FIG. 4630: DNA326231,XM_{—}071873,gen.XM_{—}071873

FIG. 4631: PRO82643

FIG. 4632: DNA326232,XM_{—}047525,gen.XM_{—}047525

FIG. 4633: DNA326233,NM_{—}000977,gen.NM_{—}000977

FIG. 4634: PRO82645

FIG. 4635: DNA326234,NM_{—}033251,gen.NM_{—}033251

FIG. 4636: PRO82646

FIG. 4637: DNA326235,XM_{—}085408,gen.XM_{—}085408

FIG. 4638: DNA326236,NM_{—}004933,gen.NM_{—}004933

FIG. 4639: PRO2198

FIG. 4640: DNA326237,XM_{—}113882,gen.XM_{—}113882

FIG. 4641: DNA326238,XM_{—}010938,gen.XM_{—}010938

FIG. 4642: DNA326239,NM_{—}006761,gen.NM_{—}006761

FIG. 4643: PRO39530

FIG. 4644A-B: DNA326240,XM_{—}017096,gen.XM_{—}017096

FIG. 4645: DNA326241,XM_{—}033714,gen.XM_{—}033714

FIG. 4646A-B: DNA326242,XM_{—}033689,gen.XM_{—}033689

FIG. 4647: DNA326243,NM_{—}002615,gen.NM_{—}002615

FIG. 4648: DNA326244,XM_{—}056082,gen.XM_{—}056082

FIG. 4649: PRO82654

FIG. 4650: DNA326245,XM_{—}008557,gen.XM_{—}008557

FIG. 4651: DNA326246,XM_{—}045183,gen.XM_{—}045183

FIG. 4652: PRO82656

FIG. 4653: DNA326247,XM_{—}113901,gen.XM_{—}113901

FIG. 4654: DNA326248,NM_{—}080822,gen.NM_{—}080822

FIG. 4655: PRO82658

FIG. 4656A-B: DNA326249,XM_{—}029438,gen.XM_{—}029438

FIG. 4657: PRO82659

FIG. 4658: DNA326250,XM_{—}008509,gen.XM_{—}008509

FIG. 4659: DNA326251,XM_{—}085687,gen.XM_{—}085687

FIG. 4660: PRO82661

FIG. 4661: DNA326252,XM_{—}027825,gen.XM_{—}027825

FIG. 4662: PRO82662

FIG. 4663: DNA326253,XM_{—}053717,gen.XM_{—}053717

FIG. 4664: PRO82663

FIG. 4665: DNA326254,NM_{—}005022,gen.NM_{—}005022

FIG. 4666: PRO62780

FIG. 4667A-B: DNA326255,XM_{—}028398,gen.XM_{—}028398

FIG. 4668: PRO82664

FIG. 4669: DNA326256,NM_{—}000018,gen.NM_{—}000018

FIG. 4670: PRO66265

FIG. 4671: DNA326257,XM_{—}008334,gen.XM_{—}008334

FIG. 4672: DNA326258,NM_{—}024297,gen.NM_{—}024297

FIG. 4673: PRO82665

FIG. 4674: DNA326259,XM_{—}113324,gen.XM_{—}113324

FIG. 4675: DNA326260,XM_{—}012676,gen.XM_{—}012676

FIG. 4676: PRO82667

FIG. 4677: DNA326261,XM_{—}085691,gen.XM_{—}085691

FIG. 4678: DNA326262,XM_{—}028417,gen.XM_{—}028417

FIG. 4679: PRO82669

FIG. 4680A-B: DNA326263,XM_{—}041964,gen.XM_{—}041964

FIG. 4681: PRO82670

FIG. 4682: DNA326264,NM_{—}019013,gen.NM_{—}019013

FIG. 4683: PRO82671

FIG. 4684: DNA326265,XM_{—}008538,gen.XM_{—}008538

FIG. 4685: PRO82672

FIG. 4686: DNA326266,XM_{—}008441,gen.XM_{—}008441

FIG. 4687: DNA97300,NM_{—}001416,gen.NM_{—}001416

FIG. 4688: PRO3647

FIG. 4689: DNA326267,NM_{—}004870,gen.NM_{—}004870

FIG. 4690: PRO82674

FIG. 4691: DNA326268,NM_{—}006942,gen.NM_{—}006942

FIG. 4692: PRO82675

FIG. 4693: DNA326269,XM_{—}008679,gen.XM_{—}008679

FIG. 4694: DNA326270,XM_{—}008231,gen.XM_{—}008231

FIG. 4695: DNA326271,XM_{—}113328,gen.XM_{—}113328

FIG. 4696: DNA326272,XM_{—}113929,gen.XM_{—}113929

FIG. 4697: DNA326273,NM_{—}001970,gen.NM_{—}001970

FIG. 4698: PRO82678

FIG. 4699: DNA297388,NM_{—}004217,gen.NM_{—}004217

FIG. 4700: PRO70812

FIG. 4701: DNA326274,XM_{—}165421,gen.XM_{—}165421

FIG. 4702: PRO82679

FIG. 4703: DNA326275,XM_{—}113325,gen.XM_{—}113325

FIG. 4704: DNA326276,XM_{—}165422,gen.XM_{—}165422

FIG. 4705: PRO49182

FIG. 4706: DNA326277,XM_{—}113931,gen.XM_{—}113931

FIG. 4707: DNA326278,XH_{—}036659,gen.XM_{—}036659

FIG. 4708: DNA103401,NM_{—}003876,gen.NM_{—}003876

FIG. 4709: PRO4729

FIG. 4710A-B: DNA326279,XM_{—}042698,gen.XM_{—}042698

FIG. 4711: PRO82683

FIG. 4712A-B: DNA326280,NM_{—}017234,gen.XM_{—}017234

FIG. 4713: DNA326281,XM_{—}165418,gen.XM_{—}165418

FIG. 4714: DNA304715,NM_{—}000987,gen.NM_{—}000987

FIG. 4715: PRO71141

FIG. 4716A-B: DNA326282,NM_{—}004618,gen.NM_{—}004618

FIG. 4717: PRO62981

FIG. 4718: DNA326283,XM_{—}085743,gen.XM_{—}085743

FIG. 4719A-B: DNA254198,NM_{—}002018,gen.NM_{—}002018

FIG. 4720: PRO49310

FIG. 4721A-B: DNA326284,XM_{—}039910,gen.XM_{—}039910

FIG. 4722: PRO82687

FIG. 4723A-C: DNA326285,XM_{—}113310,gen.XM_{—}113310

FIG. 4724: DNA326286,XM_{—}085613,gen.XM_{—}085613

FIG. 4725: DNA326287,NM_{—}006470,gen.NM_{—}006470

FIG. 4726: PRO82689

FIG. 4727: DNA326288,XM_{—}051763,gen.XM_{—}051763

FIG. 4728: DNA290292,NM_{—}018955,gen.NM_{—}018955

FIG. 4729: PRO70449

FIG. 4730: DNA326289,XM_{—}058900,gen.XM_{—}058900

FIG. 4731: PRO82691

FIG. 4732: DNA326290,XM_{—}039921,gen.XM_{—}039921

FIG. 4733: PRO82692

FIG. 4734: DNA326291,XM_{—}012549,gen.XM_{—}012549

FIG. 4735: DNA326292,XM_{—}085548,gen.XM_{—}085548

FIG. 4736: PRO82694

FIG. 4737: DNA326293,NM_{—}018019,gen.NM_{—}018019

FIG. 4738: PRO82695

FIG. 4739: DNA326294,NM_{—}138427,gen.NM_{—}138427

FIG. 4740: PRO82696

FIG. 4741: DNA326295,XM_{—}085545,gen.XM_{—}085545

FIG. 4742A-B: DNA227084,NM_{—}004176,gen.NM_{—}004176

FIG. 4743: PRO37547

FIG. 4744: DNA326296,XM_{—}012615,gen.XM_{—}012615

FIG. 4745: DNA326297,XM_{—}085722,gen.XM_{—}085722

FIG. 4746: PRO82699

FIG. 4747: DNA255414,NM_{—}018242,gen.NM_{—}018242

FIG. 4748: PRO50481

FIG. 4749: DNA326298,XM_{—}045044,gen.XM_{—}045044

FIG. 4750: DNA326299,XM_{—}008323,gen.XM_{—}008323

FIG. 4751: DNA326300,XM_{—}045535,gen.XM_{—}045535

FIG. 4752A-B: DNA326301,XM_{—}045551,gen.XM_{—}045551

FIG. 4753: PRO82702

FIG. 4754: DNA326302,XM_{—}097204,gen.XM_{—}097204

FIG. 4755: DNA326303,XM_{—}058867,gen.XM_{—}058867

FIG. 4756: PRO82704

FIG. 4757: DNA326304,XM_{—}085672,gen.XM_{—}085672

FIG. 4758: DNA326305,XM_{—}031536,gen.XM_{—}031536

FIG. 4759: PRO82706

FIG. 4760: DNA326306,XM_{—}008486,gen.XM_{—}008486

FIG. 4761: DNA326307,NM_{—}015584,gen.NM_{—}015584

FIG. 4762: PRO82707

FIG. 4763: DNA326308,NM_{—}000638,gen.NM_{—}000638

FIG. 4764: PRO82708

FIG. 4765A-B: DN6 26309,XM_{—}031466,gen.XM_{—}031466

FIG. 4766: PRO82709

FIG. 4767: DNA326310,XM_{—}031415,gen.XM_{—}031415

FIG. 4768: DNA326311,XM_{—}117066,gen.XM_{—}117066

FIG. 4769: DNA326312,XM_{—}031427,gen.XM_{—}031427

FIG. 4770: PRO82712

FIG. 4771: DNA326313,NM_{—}032322,gen.NM_{—}032322

FIG. 4772: PRO82713

FIG. 4773A-B: DNA326314,XM_{—}050101,gen.XM_{—}050101

FIG. 4774: PRO82714

FIG. 4775: DNA326315,XM_{—}056730,gen.XM_{—}056730

FIG. 4776: PRO82715

FIG. 4777: DNA326316,XM_{—}008462,gen.XM_{—}008462

FIG. 4778: DNA287427,NM_{—}002815,gen.NM_{—}002815

FIG. 4779: PRO69684

FIG. 4780: DNA326317,NM_{—}015544,gen.NM_{—}015544

FIG. 4781: PRO82717

FIG. 4782: DNA188351,NM_{—}005623,gen.NM_{—}005623

FIG. 4783: PRO21887

FIG. 4784: DNA326318,NM_{—}002878,gen.NM_{—}002878

FIG. 4785: PRO82718

FIG. 4786: DNA326319,NM_{—}133627,gen.NM_{—}133627

FIG. 4787: PRO82719

FIG. 4788: DNA326320,NM_{—}133630,gen.NM_{—}133630

FIG. 4789: PRO82720

FIG. 4790: DNA326321,NM_{—}133629,gen.NM_{—}133629

FIG. 4791: PRO82721

FIG. 4792: DNA326322,NM_{—}018096,gen.NM_{—}018096

FIG. 4793: PRO37791

FIG. 4794A-B: DNA326323,XM_{—}039474,gen.XM_{—}039474

FIG. 4795: PRO82722

FIG. 4796A-B: DNA66475,NM_{—}004448,gen.NM_{—}004448

FIG. 4797: PRO 1204

FIG. 4798: DNA326324,NM_{—}000981,gen.NM_{—}000981

FIG. 4799: PRO4738

FIG. 4800A-B: DNA326325,XM_{—}008150,gen.XM_{—}008150

FIG. 4801: DNA326326,NM_{—}000978,gen.NM_{—}000978

FIG. 4802: PRO82724

FIG. 4803: DNA326327,XM_{—}058830,gen.XM_{—}058830

FIG. 4804: PRO82725

FIG. 4805: DNA270979,NM_{—}002809,gen.NM_{—}002809

FIG. 4806: PRO59309

FIG. 4807: DNA326328,NM_{—}000422,gen.NM_{—}000422

FIG. 4808: PRO82726

FIG. 4809: DNA326329,XM_{—}008579,gen.XM_{—}008579

FIG. 4810: DNA326330,NM_{—}002276,gen.NM_{—}002276

FIG. 4811: PRO82728

FIG. 4812: DNA272889,NM_{—}002275,gen.NM_{—}002275

FIG. 4813: PRO60979

FIG. 4814: DNA326331,NM_{—}002274,gen.NM_{—}002274

FIG. 4815: PRO82729

FIG. 4816: DNA326332,NM_{—}000526,gen.NM_{—}000526

FIG. 4817: PRO82730

FIG. 4818: DNA326333,XM_{—}049937,gen.XM_{—}049937

FIG. 4819A-B: DNA326334,XM_{—}1 13334,gen.XM_{—}113334

FIG. 4820: DNA226389,NM_{—}000964,gen.NM_{—}000964

FIG. 4821: PRO36852

FIG. 4822: DNA326335,NM_{—}006455,gen.NM_{—}006455

FIG. 4823: PRO82732

FIG. 4824: DNA326336,XM_{—}113938,gen.XM_{—}113938

FIG. 4825: DNA326337,XM_{—}036465,gen.XM_{—}036465

FIG. 4826: DNA326338,XM_{—}055061,gen.XM_{—}055061

FIG. 4827A-B: DNA326339,XM_{—}036462,gen.XM_{—}036462

FIG. 4828: PRO82736

FIG. 4829: DNA326340,XM_{—}048654,gen.XM_{—}048654

FIG. 4830: DNA326341,NM_{—}025197,gen.NM_{—}025197

FIG. 4831: PRO82737

FIG. 4832: DNA326342,XM_{—}054038,gen.XM_{—}054038

FIG. 4833: PRO82738

FIG. 4834: DNA326343,NM_{—}002265,gen.NM_{—}002265

FIG. 4835: PRO82739

FIG. 4836: DNA326344,XM_{—}032201,gen.XM_{—}032201

FIG. 4837: PRO82740

FIG. 4838: DNA326345,NM_{—}012138,gen.NM_{—}012138

FIG. 4839: PRO82741

FIG. 4840: DNA326346,XM_{—}018534,gen.XM_{—}018534

FIG. 4841: DNA227873,NM_{—}001050,gen.NM_{—}001050

FIG. 4842: PRO38336

FIG. 4843: DNA270975,NM_{—}000386,gen.NM_{—}000386

FIG. 4844: PRO59305

FIG. 4845: DNA88378,NM_{—}002087,gen.NM_{—}002087

FIG. 4846: PRO2769

FIG. 4847: DNA326347,NM_{—}016016,gen.NM_{—}016016

FIG. 4848: PRO82743

FIG. 4849: DNA326348,XM_{—}012642,gen.XM_{—}012642

FIG. 4850A-B: DNA326349,NM_{—}005474,gen.NM_{—}005474

FIG. 4851: PRO82745

FIG. 4852: DNA326350,XM_{—}045901,gen.XM_{—}045901

FIG. 4853: PRO82746

FIG. 4854: DNA257428,NM_{—}032376,gen.NM_{—}032376

FIG. 4855: PRO52010

FIG. 4856: DNA326351,XM_{—}008351,gen.XM_{—}008351

FIG. 4857: DNA326352,XM_{—}032852,gen.XM_{—}032852

FIG. 4858: PRO82748

FIG. 4859: DNA326353,NM_{—}025233,gen.NM_{—}025233

FIG. 4860: PRO82749

FIG. 4861: DNA326354,XM_{—}032817,gen.XM_{—}032817

FIG. 4862: PRO82750

FIG. 4863: DNA326355,XM_{—}032813,gen.XM_{—}032813

FIG. 4864: DNA326356,XM_{—}032766,gen.XM_{—}032766

FIG. 4865: DNA326357,NM_{—}003766,gen.NM_{—}003766

FIG. 4866: PRO82753

FIG. 4867: DNA326358,XM_{—}008401,gen.XM_{—}008401

FIG. 4868: PRO82754

FIG. 4869: DNA326359,XM_{—}008402,gen.XM_{—}008402

FIG. 4870: PRO82755

FIG. 4871: DNA326360,NM_{—}017595,gen.NM_{—}017595

FIG. 4872: PRO82756

FIG. 4873: DNA326361,XM_{—}085636,gen.XM_{—}085636

FIG. 4874: PRO82757

FIG. 4875: DNA326362,NM_{—}006373,gen.NM_{—}006373

FIG. 4876: PRO82758

FIG. 4877: DNA196642,NM_{—}005440,gen.NM_{—}005440

FIG. 4878: PRO25115

FIG. 4879A-B: DNA270901,NM_{—}004247,gen.NM_{—}004247

FIG. 4880: DNA326363,XM_{—}050159,gen.XM_{—}050159

FIG. 4881: DNA326364,XM_{—}083983,gen.XM_{—}083983

FIG. 4882: PRO82760

FIG. 4883A-B: DNA326365,NM_{—}021079,gen.NM_{—}021079

FIG. 4884: PRO82761

FIG. 4885: DNA326366,NM_{—}133373,gen.NM_{—}133373

FIG. 4886: PRO82762

FIG. 4887: DNA97290,NM_{—}002512,gen.NM_{—}002512

FIG. 4888: PRO3637

FIG. 4889: DNA227071,NM_{—}000269,gen.NM_{—}000269

FIG. 4890: PRO37534

FIG. 4891: DNA227764,NM_{—}003971,gen.NM_{—}003971

FIG. 4892: PRO38227

FIG. 4893A-B: DNA326367,NM_{—}020038,gen.NM_{—}020038

FIG. 4894: PRO82763

FIG. 4895A-B: DNA326368,NM_{—}020037,gen.NM_{—}020037

FIG. 4896: PRO82764

FIG. 4897: DNA326369,XM_{—}037971,gen.XM_{—}037971

FIG. 4898: DNA254791,NM_{—}018346,gen.NM_{—}018346

FIG. 4899: PRO49888

FIG. 4900: DNA287425,NM_{—}018509,gen.NM_{—}018509

FIG. 4901: PRO69682

FIG. 4902A-B: DNA326370,XM_{—}008432,gen.XM_{—}008432

FIG. 4903: DNA88554,NM_{—}000250,gen.NM_{—}000250

FIG. 4904: PRO2839

FIG. 4905: DNA326371,XM_{—}113919,gen.XM_{—}113919

FIG. 4906: DNA326372,NM_{—}017777,gen.NM_{—}017777

FIG. 4907: PRO82768

FIG. 4908: DNA326373,NM_{—}006924,gen.NM_{—}006924

FIG. 4909: PRO82769

FIG. 4910: DNA326374,XM_{—}115480,gen.XM_{—}115480

FIG. 4911: DNA326375,NM_{—}005831,gen.NM_{—}005831

FIG. 4912: PRO59328

FIG. 4913: DNA326376,XM_{—}117061,gen.XM_{—}117061

FIG. 4914: PRO82771

FIG. 4915: DNA326377,XM_{—(}08459,gen.XM_{—}008459

FIG. 4916A-B: DNA326378,XM_{—}012651,gen.XM_{—}012651

FIG. 4917: DNA326379,NM_{—}021626,gen.NM_{—}021626

FIG. 4918: PRO302

FIG. 4919: DNA287291,NM_{—}021213,gen.NM_{—}021213

FIG. 4920: PRO69561

FIG. 4921A-B: DNA326380,NM_{—}004859,gen.NM_{—}004859

FIG. 4922: PRO82774

FIG. 4923: DNA326381,XM_{—}083966,gen.XM_{—}083966

FIG. 4924: DNA326382,XM_{—}044426,gen.XM_{—}044426

FIG. 4925: PRO82776

FIG. 4926: DNA326383,XM_{—}008253,gen.XM_{—}008253

FIG. 4927: DNA326384,XM_{—}044394,gen.XM_{—}044394

FIG. 4928: PRO10400

FIG. 4929: DNA326385,NM_{—}017647,gen.NM_{—}017647

FIG. 4930: PRO82778

FIG. 4931: DNA326386,NM_{—}007372,gen.NM_{—}007372

FIG. 4932: PRO82779

FIG. 4933: DNA326387,NM_{—}002401,gen.NM_{—}002401

FIG. 4934: PRO37764

FIG. 4935: DNA326388,XM_{—}044376,gen.XM_{—}044376

FIG. 4936A-B: DNA150457,NM_{—}006039,gen.XM_{—}044376

FIG. 4937: PRO12265

FIG. 4938: DNA326389,XM_{—}044367,gen.XM_{—}044367

FIG. 4939: DNA227055,NM_{—}002634,gen.NM_{—}002634

FIG. 4940: PRO37518

FIG. 4941: DNA326390,XM_{—}111118,gen.XM_{—}011118

FIG. 4942: DNA326391,XM_{—}055199,gen.XM_{—}055199

FIG. 4943A-B: DNA326392,XM_{—}44372,gen.XM_{—}044372

FIG. 4944: DNA326393,XM_{—}113315,gen.XM_{—}113315

FIG. 4945: DNA326394,XM_{—}012609,gen.XM_{—}012609

FIG. 4946: DNA326395,NM_{—}005220,gen.NM_{—}005220

FIG. 4947: PRO82787

FIG. 4948: DNA326396,XM_{—}085589,gen.XM_{—}085589

FIG. 4949: PRO82788

FIG. 4950: DNA326397,XM_{—}012634,gen.XM_{—}012634

FIG. 4951: DNA326398,XM_{—}085627,gen.XM_{—}085627

FIG. 4952: PRO82790

FIG. 4953: DNA150814,NM_{—}002086,gen.NM_{—}002086

FIG. 4954: PRO12806

FIG. 4955: DNA326399,NM_{—}024844,gen.NM_{—}024844

FIG. 4956: PRO82791

FIG. 4957: DNA326400,XM_{—}041583,gen.XM_{—}041583

FIG. 4958: DNA326401,XM_{—}046932,gen.XM_{—}046932

FIG. 4959: PRO82792

FIG. 4960: DNA326402,NM_{—}004524,gen.NM_{—}004524

FIG. 4961: PRO82793

FIG. 4962A-B: DNA326403,XM_{—}113951,gen.XM_{—}113951

FIG. 4963A-B: DNA88430,NM_{—}000213,gen.NM_{—}000213

FIG. 4964: PRO2788

FIG. 4965A-B: DNA326404,XM_{—}036104,gen.XM_{—}036104

FIG. 4966: PRO82794

FIG. 4967: DNA326405,NM_{—}000154,gen.NM_{—}000154

FIG. 4968: PRO82795

FIG. 4969: DNA326406,NM_{—}005324,gen.NM_{—}005324

FIG. 4970: PRO1 1403

FIG. 4971A-B: DNA326407,XM_{—}036115,gen.XM_{—}036115

FIG. 4972: PRO82796

FIG. 4973: DNA326408,XM_{—}054344,gen.XM_{—}054344

FIG. 4974: PRO82797

FIG. 4975: DNA274755,NM_{—}002766,gen.NM_{—}002766

FIG. 4976: PRO70703

FIG. 4977A-B: DNA326409,XM_{—}085531,gen.XM_{—}085531

FIG. 4978: DNA326410,XM_{—}113892,gen.XM_{—}113892

FIG. 4979: PRO82799

FIG. 4980: DNA326411,XM_{—}017578,gen.XM_{—}017578

FIG. 4981: PRO82800

FIG. 4982: DNA326412,XM_{—}036785,gen.XM_{—}036785

FIG. 4983: PRO39201

FIG. 4984: DNA326413,XM_{—}097043,gen.XM_{—}097043

FIG. 4985: DNA129504,NM_{—}001168,gen.NM_{—}001168

FIG. 4986: PRO7143

FIG. 4987: DNA326414,XM_{—}037196,gen.XM_{—}037196

FIG. 4988: DNA326415,XM_{—}037195,gen.XM_{—}037195

FIG. 4989: DNA326416,XM_{—}045104,gen.XM_{—}045104

FIG. 4990: PRO37540

FIG. 4991: DNA326417,XM_{—}085563,gen.XM_{—}085563

FIG. 4992A-B: DNA326418,XM_{—}085716,gen.XM_{—}085716

FIG. 4993: PRO82805

FIG. 4994A-B: DNA326419,XM_{—}049934,gen.XM_{—}049934

FIG. 4995: DNA326420,XM_{—}049931,gen.XM_{—}049931

FIG. 4996A-B: DNA326421,XM_{—}045581,gen.XM_{—}045581

FIG. 4997: PRO82807

FIG. 4998: DNA326422,XM_{—}113945,gen.XM_{—}113945

FIG. 4999: DNA326423,XM_{—}046481,gen.XM_{—}046481

FIG. 5000: DNA326424,XM_{—}097195,gen.XM_{—}097195

FIG. 5001: DNA326425,XM_{—}097193,gen.XM_{—}097193

FIG. 5002: DNA326426,NM_{—}004309,gen.NM_{—}004309

FIG. 5003: PRO61246

FIG. 5004: DNA326427,XM_{—}046472,gen.XM_{—}046472

FIG. 5005: PRO82812

FIG. 5006: DNA326428,NM_{—}016286,gen.NM_{—}016286

FIG. 5007: PRO82813

FIG. 5008: DNA326429,NM_{—}004127,gen.NM_{—}004127

FIG. 5009: PRO82814

FIG. 5010A-C: DNA326430,XM_{—}113943,gen.NM_{—}004127

FIG. 5011: DNA326431,XM_{—}113330,gen.XM_{—}113330

FIG. 5012: PRO82816

FIG. 5013: DNA326432,XM_{—}113303,gen.XM_{—}113303

FIG. 5014: DNA287234,NM_{—}031968,gen.NM_{—}031968

FIG. 5015: PRO69513

FIG. 5016: DNA326433,NM_{—}022158,gen.NM_{—}022158

FIG. 5017: PRO82818

FIG. 5018: DNA326434,XM_{—}038424,gen.XM_{—}038424

FIG. 5019: DNA326435,XM_{—}085735,gen.XM_{—}085735

FIG. 5020: DNA326436,XM_{—}046765,gen.XM_{—}046765

FIG. 5021: DNA326437,XM_{—}046769,gen.XM_{—}046769

FIG. 5022: DNA326438,XM_{—}046767,gen.XM_{—}046767

FIG. 5023: DNA273694,NM_{—}006101,gen.NM_{—}006101

FIG. 5024: PRO61661

FIG. 5025A-B: DNA326439,XM_{—}028744,gen.XM_{—}028744

FIG. 5026: DNA326440,XM_{—}165954,gen.XM_{—}165954

FIG. 5027: DNA326441,XM_{—}041678,gen.XM_{—}041678

FIG. 5028: DNA326442,XM_{—}1 13343,gen.XM_{—}113343

FIG. 5029: PRO82825

FIG. 5030: DNA326443,XM_{—}067325,gen.XM_{—}067325

FIG. 5031: DNA326444,XM_{—}012741,gen.XM_{—}012741

FIG. 5032: DNA326445,NM_{—}014214,gen.NM_{—}014214

FIG. 5033: PRO82828

FIG. 5034A-B: DNA326446,XM_{—}035640,gen.XM_{—}035640

FIG. 5035: PRO82829

FIG. 5036: DNA326447,XM_{—}016382,gen.XM_{—}016382

FIG. 5037: DNA326448,NM_{—}032933,gen.NM_{—}032933

FIG. 5038: PRO82831

FIG. 5039: DNA274690,NM_{—}006938,gen.NM_{—}006938

FIG. 5040A-B: DNA88457,NM_{—}000227,gen.NM_{—}000227

FIG. 5041: PRO2799

FIG. 5042: DNA326449,XM_{—}085791,gen.XM_{—}085791

FIG. 5043: DNA326450,XM_{—}085789,gen.XM_{—}085789

FIG. 5044: PRO82833

FIG. 5045: DNA326451,XM_{—}085790,gen.XM_{—}085790

FIG. 5046: DNA326452,XM_{—}015755,gen.XM_{—}015755

FIG. 5047: PRO82835

FIG. 5048: DNA326453,XM_{—}097232,gen.XM_{—}097232

FIG. 5049: DNA326454,XM_{—}085788,gen.XM_{—}085788

FIG. 5050: DNA88281,NM_{—}001944,gen.NM_{—}001944

FIG. 5051: PRO2267

FIG. 5052: DNA271841,NM_{—}003787,gen.NM_{—}003787

FIG. 5053: PRO60121

FIG. 5054: DNA326455,XM_{—}008723,gen.XM_{—}008723

FIG. 5055: DNA326456,XM_{—}084007,gen.XM_{—}084007

FIG. 5056: DNA256813,NM_{—}018255,gen.NM_{—}018255

FIG. 5057: PRO51744

FIG. 5058: DNA326457,XM_{—}085775,gen.XM_{—}085775

FIG. 5059: PRO82840

FIG. 5060: DNA326458,NM_{—}138443,gen.NM_{—}138443

FIG. 5061: PRO82841

FIG. 5062: DNA326459,XM_{—}038872,gen.XM_{—}038872

FIG. 5063: PRO82842

FIG. 5064: DNA326460,XM_{—}086779,gen.XM_{—}086779

FIG. 5065: DNA326461,XM_{—}167363,gen.XM_{—}167363

FIG. 5066: DNA326462,XM_{—}031944,gen.XM_{—}031944

FIG. 5067: DNA326463,NM_{—}000985,gen.NM_{—}000985

FIG. 5068: PRO82846

FIG. 5069: DNA326464,NM_{—}002396,gen.NM_{—}002396

FIG. 5070: PRO61113

FIG. 5071: DNA326465,XM_{—}166288,gen.XM_{—}166288

FIG. 5072: DNA326466,NM_{—}004539,gen.NM_{—}004539

FIG. 5073: PRO60800

FIG. 5074: DNA326467,XM_{—}006937,gen.XM_{—}006937

FIG. 5075: DNA326468,XM_{—}085779,gen.XM_{—}085779

FIG. 5076: DNA326469,XM_{—}011089,gen.XM_{—}011089

FIG. 5077: PRO82850

FIG. 5078: DNA326470,XM_{—}169540,gen.XM_{—}169540

FIG. 5079: PRO82851

FIG. 5080: DNA326471,XM_{—}167008,gen.XM_{—}167008

FIG. 5081: PRO82852

FIG. 5082: DNA326472,XM_{—}048471,gen.XM_{—}048471

FIG. 5083A-B: DNA326473,XM_{—}008812,gen.XM_{—}008812

FIG. 5084A-B: DNA326474,XM_{—}117096,gen.XM_{—}117096

FIG. 5085: PRO82855

FIG. 5086: DNA326475,NM_{—}002385,gen.NM_{—}002385

FIG. 5087: PRO82856

FIG. 5088: DNA326476,XM_{—}015241,gen.XM_{—}015241

FIG. 5089A-B: DNA326477,XM_{—}008695,gen.XM_{—}008695

FIG. 5090A-B: DNA326478,XM_{—}041872,gen.XM_{—}041872

FIG. 5091: PRO82859

FIG. 5092: DNA326479,XM_{—}051586,gen.XM_{—}051586

FIG. 5093: DNA326480,NM_{—}003712,gen.NM_{—}003712

FIG. 5094: PRO1077

FIG. 5095: DNA326481,XM_{—}042018,gen.XM_{—}042018

FIG. 5096: PRO2560

FIG. 5097: DNA326482,XM_{—}114018,gen.XM_{—}114018

FIG. 5098: DNA326483,NM_{—}017876,gen.NM_{—}017876

FIG. 5099: PRO82861

FIG. 5100: DNA326484,NM_{—}031990,gen.NM_{—}031990

FIG. 5101: PRO82862

FIG. 5102: DNA326485,NM_{—}002819,gen.NM_{—}002819

FIG. 5103: PRO62899

FIG. 5104: DNA326486,NM_{—}005224,gen.NM_{—}005224

FIG. 5105: PRO82863

FIG. 5106: DNA326487,XM_{—}037565,gen.XM_{—}037565

FIG. 5107: PRO82864

FIG. 5108: DNA326488,XM_{—}092042,gen.XM_{—}092042

FIG. 5109: DNA326489,XM_{—}037572,gen.XM_{—}037572

FIG. 5110: DNA326490,XM_{—}009279,gen.XM_{—}009279

FIG. 5111: PRO82867

FIG. 5112: DNA326491,NM_{—}002085,gen.NM_{—}002085

FIG. 5113A-B: DNA326492,XM_{—}009277,gen.XM_{—}009277

FIG. 5114: DNA326493,XM_{—}012913,gen.XM_{—}012913

FIG. 5115: DNA274101,NM_{—}001687,gen.NM_{—}001687

FIG. 5116: PRO62039

FIG. 5117: DNA326494,XM_{—}028067,gen.XM_{—}028067

FIG. 5118: PRO82871

FIG. 5119: DNA326495,XM_{—}028064,gen.XM_{—}028064

FIG. 5120: DNA326496,NM_{—}024407,gen.NM_{—}024407

FIG. 5121: PRO82872

FIG. 5122: DNA326497,NM_{—}000156,gen.NM_{—}000156

FIG. 5123: PRO58046

FIG. 5124: DNA326498,NM_{—}138924,gen.NM_{—}138924

FIG. 5125: PRO82873

FIG. 5126: DNA326499,NM_{—}001018,gen.NM_{—}001018

FIG. 5127: PRO10485

FIG. 5128: DNA326500,XM_{—}086101,gen.XM_{—}086101

FIG. 5129: PRO82874

FIG. 5130: DNA326501,XM_{—}086102,gen.XM_{—}086102

FIG. 5131: DNA326502,XM_{—}047584,gen.XM_{—}047584

FIG. 5132A-B: DNA326503,XM_{—}047600,gen.XM_{—}047600

FIG. 5133: PRO38496

FIG. 5134: DNA326504,XM_{—}097420,gen.XM_{—}097420

FIG. 5135A-B: DNA326505,XM_{—}030721,gen.XM_{—}030721

FIG. 5136: PRO82877

FIG. 5137: DNA326506,XM_{—}030720,gen.XM_{—}030720

FIG. 5138: DNA326507,NM_{—}031213,gen.NM_{—}031213

FIG. 5139: PRO82879

FIG. 5140: DNA326508,XM_{—}039723,gen.XM_{—}039723

FIG. 5141: DNA326509,NM_{—}001319,gen.NM_{—}001319

FIG. 5142: PRO82881

FIG. 5143: DNA326510,NM_{—}017797,gen.NM_{—}017797

FIG. 5144: PRO82882

FIG. 5145: DNA326511,XM_{—}030714,gen.XM_{—}030714

FIG. 5146: DNA256555,NM_{—}017572,gen.NM_{—}017572

FIG. 5147: PRO51586

FIG. 5148A-B: DNA326512,NM_{—}003938,gen.NM_{—}003938

FIG. 5149: PRO82884

FIG. 515OA-B: DNA326513,XM_{—}046822,gen.XM_{—}046822

FIG. 5151: PRO82885

FIG. 5152: DNA326514,NM_{—}007165,gen.NM_{—}007165

FIG. 5153: PRO82886

FIG. 5154: DNA287636,NM_{—}004152,gen.NM_{—}004152

FIG. 5155: DNA326515,NM_{—}012458,gen.NM_{—}012458

FIG. 5156: PRO82887

FIG. 5157: DNA326516,NM_{—}032737,gen.NM_{—}032737

FIG. 5158: PRO82888

FIG. 5159: DNA326517,XM_{—}030485,gen.XM_{—}030485

FIG. 5160: DNA326518,XM_{—}046934,gen.XM_{—}046934

FIG. 5161: DNA326519,NM_{—}003021,gen.NM_{—}003021

FIG. 5162: PRO62302

FIG. 5163: DNA326520,XM_{—}055686,gen.XM_{—}055686

FIG. 5164: PRO37951

FIG. 5165: DNA326521,XM_{—}009222,gen.XM_{—}009222

FIG. 5166: DNA326522,XM_{—}052635,gen.XM_{—}052635

FIG. 5167: PRO82892

FIG. 5168: DNA326523,XM_{—}052661,gen.XM_{—}052661

FIG. 5169: DNA326524,NM_{—}016263,gen.NM_{—}016263

FIG. 5170: PRO82893

FIG. 5171: DNA326525,NM_{—}006339,gen.NM_{—}006339

FIG. 5172: PRO82894

FIG. 5173: DNA326526,NM_{—}032753,gen.NM_{—}032753

FIG. 5174: PRO82895

FIG. 5175: DNA326527,XM_{—}056421,gen.XM_{—}056421

FIG. 5176A-B: DNA326528,XM_{—}031917,gen.XM_{—}031917

FIG. 5177: PRO82897

FIG. 5178: DNA326529,NM_{—}001961,gen.NM_{—}001961

FIG. 5179: PRO62225

FIG. 5180: DNA326530,XM_{—}016871,gen.XM_{—}016871

FIG. 5181: DNA326531,NM_{—}1016539,gen.NM_{—}16539

FIG. 5182: PRO82899

FIG. 5183: DNA326532,XM_{—}117122,gen.XM_{—}117122

FIG. 5184: DNA326533,XM_{—}031857,gen.XM_{—}031857

FIG. 5185: PRO82901

FIG. 5186: DNA326534,NM_{—}024333,gen.NM_{—}924333

FIG. 5187: PRO82902

FIG. 5188: DNA326535,NM_{—}003025,gen.NM_{—}003025

FIG. 5189: PRO82903

FIG. 5190: DNA326536,NM_{—}025241,gen.NM_{—}025241

FIG. 5191: PRO82904

FIG. 5192: DNA326537,XM_{—}035638,gen.XM_{—}035638

FIG. 5193: PRO82905

FIG. 5194A-B: DNA326538,XM_{—}035636,gen.XM_{—}035636

FIG. 5195: DNA326539,XM_{—}012862,gen.XM_{—}012862

FIG. 5196A-B: DNA326540,XM_{—}035627,gen.XM_{—}035627

FIG. 5197A-B: DNA326541,XM_{—}035625,gen.XM_{—}035625

FIG. 5198: PRO82909

FIG. 5199: DNA274761,NM_{—}014649,gen.NM_{—}014649

FIG. 5200: PRO62531

FIG. 5201: DNA272421,NM_{—}006012,gen.NM_{—}006012

FIG. 5202: PRO60674

FIG. 5203: DNA326542,NM_{—}003685,gen.NM_{—}003685

FIG. 5204: PRO82910

FIG. 5205A-B: DNA326543,XM_{—}009010,gen.XM_{—}009010

FIG. 5206: DNA270315,NM_{—}004240,gen.NM_{—}004240

FIG. 5207: PRO58702

FIG. 5208: DNA326544,NM_{—}005490,gen.NM_{—}005490

FIG. 5209: PRO201

FIG. 5210: DNA326546,XM_{—}044619,gen.XM_{—}044619

FIG. 5211: PRO82912

FIG. 5212: DNA326547,XM_{—}012798,gen.XM_{—}012798

FIG. 5213: DNA326548,XM_{—}044608,gen.XM_{—}044608

FIG. 5214: DNA326549,NM_{—}003624,gen.NM_{—}003624

FIG. 5215: PRO82915

FIG. 5216: DNA326550,NM_{—}016579,gen.NM_{—}016579

FIG. 5217: PRO224

FIG. 5218A-B: DNA326551,XM_{—}048351,gen.XM_{—}048351

FIG. 5219: DNA326552,XM_{—}048364,gen.XM_{—}048364

FIG. 5220: PRO82917

FIG. 5221: DNA326553,XM_{—}091938,gen.XM_{—}091938

FIG. 5222: DNA326554,XM_{—}097300,gen.XM_{—}097300

FIG. 5223: DNA326555,XM_{—}049282,gen.XM_{—}049282

FIG. 5224: PRO82920

FIG. 5225: DNA326556,XM_{—}058232,gen.XM_{—}058232

FIG. 5226: DNA326557,XM_{—}045151,gen.XM_{—}045151

FIG. 5227A-B: DNA326558,XM_{—}050435,gen.XM_{—}050435

FIG. 5228: PRO82923

FIG. 5229: DNA326559,XM_{—}113988,gen.XM_{—}113988

FIG. 5230: DNA326560,NM_{—}058164,gen.NM_{—}058164

FIG. 5231: PRO82925

FIG. 5232: DNA227280,NM_{—}020230,gen.NM_{—}020230

FIG. 5233: PRO37743

FIG. 5234: DNA270621,NM_{—}003755,gen.NM_{—}003755

FIG. 5235: PRO58991

FIG. 5236: DNA326561,XM_{—}049502,gen.XM_{—}049502

FIG. 5237: DNA326562,NM_{—}007065,gen.NM_{—}007065

FIG. 5238: PRO63226

FIG. 5239: DNA326563,XM_{—}049561,gen.XM_{—}049561

FIG. 5240: DNA326564,XM_{—}017204,gen.XM_{—}017204

FIG. 5241: DNA326565,NM_{—}005498,gen.NM_{—}005498

FIG. 5242: PRO62112

FIG. 5243: DNA326566,XM_{—}008887,gen.XM_{—}008887

FIG. 5244: DNA326567,XM_{—}085862,gen.XM_{—}085862

FIG. 5245: PRO82930

FIG. 5246: DNA326568,XM_{—}084014,gen.XM_{—}084014

FIG. 5247A-B: DNA326569,XM_{—}032710,gen.XM_{—}032710

FIG. 5248: DNA326570,XM_{—}032719,gen.XM_{—}032719

FIG. 5249: PRO82933

FIG. 5250: DNA326571,NM_{—}024029,gen.NM_{—}024029

FIG. 5251: PRO23794

FIG. 5252: DNA326572,XM_{—}032724,gen.XM_{—}032724

FIG. 5253: PRO82934

FIG. 5254A-B: DNA326573,NM_{—}003072,gen.NM_{—}003072

FIG. 5255: PRO82935

FIG. 5256A-B: DNA326574,XM_{—}009082,gen.XM_{—}009082

FIG. 5257: DNA326575,XM_{—}032774,gen.XM_{—}032774

FIG. 5258: DNA218271,NM_{—}000121,gen.NM_{—}000121

FIG. 5259: PRO34323

FIG. 5260: DNA326576,XM_{—}057074,gen.XM_{—}057074

FIG. 5261: DNA326577,XM_{—}032782,gen.XM_{—}032782

FIG. 5262: DNA326578,NM_{—}032377,gen.NM_{—}032377

FIG. 5263: PRO82939

FIG. 5264: DNA326579,XM_{—}015697,gen.XM_{—}015697

FIG. 5265: PRO82940

FIG. 5266: DNA326580,XM_{—}010156,gen.XM_{—}010156

FIG. 5267: DNA326581,NM_{—}001930,gen.NM_{—}001930

FIG. 5268: PRO58446

FIG. 5269: DNA326582,NM_{—}013406,gen.NM_{—}013406

FIG. 5270: DNA326583,NM_{—}013407,gen.NM_{—}013407

FIG. 5271: PRO82943

FIG. 5272: DNA103320,NM_{—}002229,gen.NM_{—}002229

FIG. 5273: PRO4650

FIG. 5274: DNA326584,XM_{—}009063,gen.XM_{—}009063

FIG. 5275: PRO82944

FIG. 5276: DNA326585,XM_{—}085917,gen.XM_{—}085917

FIG. 5277: DNA274034,NM_{—}006397,gen.NM_{—}006397

FIG. 5278: PRO61977

FIG. 5279: DNA287243,NM_{—}004461,gen.NM_{—}004461

FIG. 5280: PRO69518

FIG. 5281: DNA326586,XM_{—}032020,gen.XM_{—}032020

FIG. 5282: PRO2718

FIG. 5283: DNA326587,NM_{—}005053,gen.NM_{—}005053

FIG. 5284: PRO22613

FIG. 5285: DNA326588,XM_{—}085916,gen.XM_{—}085916

FIG. 5286: DNA326589,NM_{—}017722,gen.NM_{—}017722

FIG. 5287: PRO82947

FIG. 5288: DNA326590,NM_{—}003765,gen.NM_{—}003765

FIG. 5289: PRO82948

FIG. 5290: DNA326591,XM_{—}051364,gen.XM_{—}051364

FIG. 5291: PRO82949

FIG. 5292: DNA326592,XM_{—}031345,gen.XM_{—}031345

FIG. 5293: PRO82950

FIG. 5294: DNA326593,XM_{—}113352,gen.XM_{—}113352

FIG. 5295: DNA326594,XM_{—}058967,gen.XM_{—}058967

FIG. 5296: PRO82952

FIG. 5297: DNA326595,XM_{—}085909,gen.XM_{—}085909

FIG. 5298: DNA269894,NM_{—}002730,gen.NM_{—}002730

FIG. 5299: PRO58292

FIG. 5300: DNA326596,NM_{—}018154,gen.NM_{—}018154

FIG. 5301: PRO82954

FIG. 5302: DNA326597,XM_{—}031276,gen.XM_{—}031276

FIG. 5303: DNA326598,XM_{—}031273,gen.XM_{—}031273

FIG. 5304: PRO82956

FIG. 5305: DNA326599,XM_{—}031263,gen.XM_{—}031263

FIG. 5306: PRO82957

FIG. 5307: DNA326600,XM_{—}031251,gen.XM_{—}031251

FIG. 5308: DNA326601,NM_{—}006844,gen.NM_{—}006844

FIG. 5309: PRO82958

FIG. 5310A-C: DNA326602,XM_{—}009303,gen.XM_{—}009303

FIG. 5311: DNA326603,XM_{—}086074,gen.XM_{—}086074

FIG. 5312: DNA269630,NM_{—}003290,gen.NM_{—}003290

FIG. 5313: PRO58042

FIG. 5314: DNA326604,NM_{—}005370,gen.NM_{—}005370

FIG. 5315: PRO12130

FIG. 5316: DNA326605,XM_{—}113348,gen.XM_{—}113348

FIG. 5317: DNA326606,NM_{—}032207,gen.NM_{—}032207

FIG. 5318: PRO82962

FIG. 5319A-B: DNA326607,NM_{—}006387,gen.NM_{—}006387

FIG. 5320: PRO82963

FIG. 5321: DNA326608,NM_{—}024881,gen.NM_{—}024881

FIG. 5322: PRO82964

FIG. 5323: DNA326609,NM_{—}024104,gen.NM_{—}024104

FIG. 5324: PRO82965

FIG. 5325A-C: DNA326610,XM_{—}008854,gen.XM_{—}008854

FIG. 5326: DNA326611,NM_{—}014173,gen.NM_{—}014173

FIG. 5327: PRO82967

FIG. 5328: DNA287240,NM_{—}004335,gen.NM_{—}004335

FIG. 5329: PRO29371

FIG. 5330: DNA326612,XM_{—}050660,gen.XM_{—}050660

FIG. 5331: DNA326613,XM_{—}086116,gen.XM_{—}086116

FIG. 5332: DNA326614,NM_{—}018174,gen.NM_{—}018174

FIG. 5333: PRO82970

FIG. 5334: DNA326615,NM_{—}000980,gen.NM_{—}000980

FIG. 5335: PRO82971

FIG. 5336: DNA326616,XM_{—}055230,gen.XM_{—}055230

FIG. 5337: DNA326617,XM_{—}012179,gen.XM_{—}012179

FIG. 5338A-B: DNA326618,XM_{—}009293,gen.XM_{—}009293

FIG. 5339: DNA326619,XM_{—}038146,gen.XM_{—}038146

FIG. 5340: PRO82975

FIG. 5341: DNA326620,XM_{—}092046,gen.XM_{—}092046

FIG. 5342: PRO82976

FIG. 5343: DNA326621,XM_{—}038098,gen.XM_{—}038098

FIG. 5344: PRO82977

FIG. 5345: DNA326622,NM_{—}032627,gen.NM_{—}032627

FIG. 5346: PRO82978

FIG. 5347: DNA326623,XM_{—}165960,gen.XM_{—}165960

FIG. 5348: PRO82979

FIG. 5349: DNA326624,XM_{—}114004,gen.XM_{—}114004

FIG. 5350: DNA326625,NM_{—}012181,gen.NM_{—}012181

FIG. 5351: PRO82980

FIG. 5352: DNA227249,NM_{—}007263,gen.NM_{—}007263

FIG. 5353: PRO37712

FIG. 5354: DNA326626,XM_{—}018515,gen.XM_{—}018515

FIG. 5355: DNA326627,NM_{—}033415,gen.NM_{—}033415

FIG. 5356: PRO82982

FIG. 5357: DNA326628,XM_{—}009330,gen.XM_{—}009330

FIG. 5358: DNA326629,NM_{—}134440,gen.NM_{—}134440

FIG. 5359: PRO82983

FIG. 5360: DNA326630,NM_{—}003721,gen.NM_{—}003721

FIG. 5361: PRO59220

FIG. 5362: DNA326631,NM_{—}015965,gen.NM_{—}015965

FIG. 5363: PRO82984

FIG. 5364: DNA326632,XM_{—}016378,gen.XM_{—}016378

FIG. 5365: PRO82985

FIG. 5366: DNA326633,XM_{—}114027,gen.XM_{—}114027

FIG. 5367: DNA326634,XM_{—}165963,gen.XM_{—}165963

FIG. 5368: PRO82987

FIG. 5369: DNA326635,XM_{—}015769,gen.XM_{—}015769

FIG. 5370: DNA326636,XM_{—}012812,gen.XM_{—}012812

FIG. 5371: DNA326637,XM_{—}085971,gen.XM_{—}085971

FIG. 5372: DNA326638,XM_{—}037662,gen.XM_{—}037662

FIG. 5373: PRO82991

FIG. 5374: DNA326639,NM_{—}001238,gen.NM_{—}001238

FIG. 5375: PRO82992

FIG. 5376: DNA326640,NM_{—}057182,gen.NM_{—}057182

FIG. 5377: PRO4756

FIG. 5378: DNA326641,XM_{—}009180,gen.XM_{—}009180

FIG. 5379: DNA326642,XM_{—}117118,gen.XM_{—}117118

FIG. 5380: DNA326643,XM_{—}092049,gen.XM_{—}092049

FIG. 5381: PRO82995

FIG. 5382: DNA326644,XM_{—}028672,gen.XM_{—}028672

FIG. 5383: DNA326645,XM_{—}028666,gen.XM_{—}028666

FIG. 5384: DNA326646,XM_{—}009338,gen.XM_{—}009338

FIG. 5385: DNA326647,XM_{—}048258,gen.XM_{—}048258

FIG. 5386: PRO82998

FIG. 5387: DNA256836,NM_{—}018468,gen.NM_{—}018468

FIG. 5388: PRO51767

FIG. 5389: DNA326648,NM_{—}024321,gen.NM_{—}024321

FIG. 5390: PRO82999

FIG. 5391A-B: DNA326649,XM_{—}049237,gen.XM_{—}049237

FIG. 5392: PRO83000

FIG. 5393: DNA326650,NM_{—}032635,gen.NM_{—}032635

FIG. 5394: PRO23845

FIG. 5395: DNA326651,XM_{—}115615,gen.XM_{—}115615

FIG. 5396A-B: DNA326652,XM_{—}091984,gen.XM_{—}091984

FIG. 5397: PRO83002

FIG. 5398: DNA326653,XM_{—}085986,gen.XM_{—}085986

FIG. 5399: DNA326654,XM_{—}032285,gen.XM_{—}032285

FIG. 5400: PRO83004

FIG. 5401: DNA326655,NM_{—}002812,gen.NM_{—}002812

FIG. 5402: PRO83005

FIG. 5403A-E: DNA326656,XM_{—}029455,gen.XM_{—}029455

FIG. 5404: DNA326657,XM_{—}029450,gen.XM_{—}029450

FIG. 5405: PRO83007

FIG. 5406: DNA326658,XM_{—}009149,gen.XM_{—}009149

FIG. 5407: PRO62500

FIG. 5408: DNA326659,XM_{—}056602,gen.XM_{—}056602

FIG. 5409: DNA326660,NM_{—}012237,gen.NM_{—}012237

FIG. 5410: PRO83008

FIG. 5411: DNA326661,NM_{—}030593,gen.NM_{—}030593

FIG. 5412: PRO83009

FIG. 5413: DNA326662,NM_{—}017827,gen.NM_{—}017827

FIG. 5414: PRO83010

FIG. 5415: DNA326663,NM_{—}021107,gen.NM_{—}021107

FIG. 5416: PRO83011

FIG. 5417: DNA326664,NM_{—}033363,gen.NM_{—}033363

FIG. 5418: PRO83012

FIG. 5419: DNA326665,XM_{—}059045,gen.XM_{—}059045

FIG. 5420: PRO83013

FIG. 5421: DNA273474,NM_{—}005884,gen.NM_{—}005884

FIG. 5422: PRO61458

FIG. 5423: DNA326666,XM_{—}046090,gen.XM_{—}046090

FIG. 5424: PRO83014

FIG. 5425: DNA326667,XM_{—}086004,gen.XM_{—}086004

FIG. 5426: DNA272347,NM_{—}001020,gen.NM_{—}001020

FIG. 5427: PRO60603

FIG. 5428A-B: DNA326668,NM_{—}003169,gen.NM_{—}003169

FIG. 5429: PRO12822

FIG. 5430: DNA326669,XM_{—}053074,gen.XM_{—}053074

FIG. 5431: PRO83016

FIG. 5432: DNA326670,NM_{—}016941,gen.NM_{—}016941

FIG. 5433: PRO83017

FIG. 5434: DNA256840,NM_{—}004714,gen.NM_{—}004714

FIG. 5435: PROS 1771

FIG. 5436: DNA326671,NM_{—}001436,gen.NM_{—}001436

FIG. 5437: PRO83018

FIG. 5438: DNA326672,XM_{—}016410,gen.XM_{—}016410

FIG. 5439: DNA326673,XM_{—}012860,gen.XM_{—}012860

FIG. 5440: DNA326674,XM_{—}097365,gen.XM_{—}097365

FIG. 5441: DNA274139,NM_{—}006503,gen.NM_{—}006503

FIG. 5442: PRO62075

FIG. 5443: DNA326675,XM_{—}009203,gen.XM_{—}009203

FIG. 5444: DNA326676,XM_{—}047409,gen.XM_{—}047409

FIG. 5445: DNA326677,XM_{—}047376,gen.XM_{—}047376

FIG. 5446A-B: DNA326678,XM_{—}047374,gen.XM_{—}047374

FIG. 5447: DNA326679,XM_{—}059052,gen.XM_{—}059052

FIG. 5448: DNA273600,NM_{—}004596,gen.NM_{—}004596

FIG. 5449: PRO61575

FIG. 5450: DNA326680,XM_{—}030914,gen.XM_{—}030914

FIG. 5451: DNA326681,NM_{—}052848,gen.NM_{—}052848

FIG. 5452: PRO83027

FIG. 5453: DNA326682,XM_{—}008912,gen.XM_{—}008912

FIG. 5454: DNA326683,NM_{—}020158,gen.NM_{—}20158

FIG. 5455: PRO83029

FIG. 5456: DNA326684,XM_{—}030901,gen.XM_{—}030901

FIG. 5457: PRO83030

FIG. 5458: DNA326685,NM_{—}018035,gen.NM_{—}018035

FIG. 5459: PRO83031

FIG. 5460: DNA326686,XM_{—}085874,gen.XM_{—}085874

FIG. 5461: DNA326687,XM_{—}085875,gen.XM_{—}085875

FIG. 5462: DNA326688,XM_{—}085876,gen.XM_{—}085876

FIG. 5463: DNA326689,XM_{—}058949,gen.XM_{—}058949

FIG. 5464: PRO83035

FIG. 5465: DNA326690,XM_{—}030895,gen.XM_{—}030895

FIG. 5466: DNA326691,XM_{—}1 15603,gen.XM_{—}115603

FIG. 5467: PRO083037

FIG. 5468: DNA326692,NM_{—}001022,gen.NM_{—}001022

FIG. 5469: PRO83038

FIG. 5470: DNA326693,NM_{—}004706,gen.NM_{—}004706

FIG. 5471: PRO83039

FIG. 5472: DNA326694,XM_{—}008878,gen.XM_{—}008878

FIG. 5473: PRO83040

FIG. 5474: DNA326695,NM_{—}022752,gen.NM_{—}022752

FIG. 5475: PRO83041

FIG. 5476: DNA151808,NM_{—}006494,gen.NM_{—}006494

FIG. 5477: PRO12892

FIG. 5478: DNA326696,NM_{—}001816,gen.NM_{—}001816

FIG. 5479: PRO34151

FIG. 5480: DNA326697,NM_{—}000554,gen.NM_{—}000554

FIG. 5481: PRO83042

FIG. 5482: DNA326698,XM_{—}049920,gen.XM_{—}049920

FIG. 5483: DNA326699,XM_{—}055859,gen.XM_{—}055859

FIG. 5484A-B: DNA326700,XM_{—}009125,gen.XM_{—}009125

FIG. 5485: DNA326701,XM_{—}008860,gen.XM_{—}008860

FIG. 5486: DNA326702,XM_{—}009036,gen.XM_{—}009036

FIG. 5487: DNA326703,XM_{—}085950,gen.XM_{—}085950

FIG. 5488: DNA326704,XM_{—}028263,gen.XM_{—}028263

FIG. 5489: DNA326705,XM_{—}085928,gen.XM_{—}085928

FIG. 5490: PRO36963

FIG. 5491: DNA326706,XM_{—}028267,gen.XM_{—}028267

FIG. 5492: DNA326707,NM_{—}013403,gen.NM_{—}013403

FIG. 5493: PRO83050

FIG. 5494: DNA103580,NM_{—}001743,gen.NM_{—}001743

FIG. 5495: PRO4904

FIG. 5496: DNA326708,XM_{—}009126,gen.XM_{—}009126

FIG. 5497: DNA326709,NM_{—}006247,gen.NM_{—}006247

FIG. 5498: PRO25881

FIG. 5499: DNA326710,NM_{—}003370,gen.NM_{—}003370

FIG. 5500: PRO83052

FIG. 5501: DNA326711,XM_{—}085856,gen.XM_{—}085856

FIG. 5502: DNA150784,NM_{—}001983,gen.NM_{—}001983

FIG. 5503: PRO 12800

FIG. 5504: DNA270931,NM_{—}012099,gen.NM_{—}012099

FIG. 5505: PRO59264

FIG. 5506A-B: DNA257531,NM_{—}031417,gen.NM_{—}031417

FIG. 5507: PRO52101

FIG. 5508: DNA326712,NM_{—}001294,gen.NM_{—}001294

FIG. 5509: PRO83054

FIG. 5510: DNA326713,XM_{—}097274,gen.XM_{—}097274

FIG. 5511: DNA88084,NM_{—}000041,gen.NM_{—}000041

FIG. 5512: PRO2644

FIG. 5513: DNA256533,NM_{—}006114,gen.NM_{—}006114

FIG. 5514: PRO51565

FIG. 5515: DNA251057,NM_{—}002856,gen.NM_{—}002856

FIG. 5516: PRO47354

FIG. 5517: DNA226011,NM_{—}005581,gen.NM_{—}005581

FIG. 5518: PRO36474

FIG. 5519: DNA326714,NM_{—}012116,gen.NM_{—}012116

FIG. 5520: PRO83056

FIG. 5521: DNA326715,XM_{—}097275,gen.XM_{—}097275

FIG. 5522: DNA326716,XM_{—}008851,gen.XM_{—}008851

FIG. 5523: DNA274289,NM_{—}016440,gen.NM_{—}016440

FIG. 5524: PRO62212

FIG. 5525: DNA326717,NM_{—}012068,gen.NM_{—}012068

FIG. 5526: PRO83059

FIG. 5527: DNA326718,XM_{—}085927,gen.XM_{—}085927

FIG. 5528: DNA326719,XM_{—}084023,gen.XM_{—}084023

FIG. 5529: DNA326720,XM_{—}167530,gen.XM_{—}167530

FIG. 5530: DNA326721,XM_{—}114025,gen.XM_{—}114025

FIG. 5531: DNA326722,XM_{—}008985,gen.XM_{—}008985

FIG. 5532: DNA326723,NM_{—}030973,gen.NM_{—}030973

FIG. 5533: PRO83065

FIG. 5534: DNA326724,NM_{—}025129,gen.NM_{—}025129

FIG. 5535: PRO83066

FIG. 5536: DNA326725,NM_{—}014203,gen.NM_{—}014203

FIG. 5537: DNA326726,XM_{—}085934,gen.XM_{—}085934

FIG. 5538: PRO83068

FIG. 5539: DNA326727,NM_{—}001536,gen.NM_{—}001536

FIG. 5540: PRO83069

FIG. 5541: DNA326728,XM_{—}165432,gen.XM_{—}165432

FIG. 5542: DNA274823,NM_{—}001571,gen.NM_{—}001571

FIG. 5543: PRO62582

FIG. 5544A-B: DNA326729,XM_{—}046313,gen.XM_{—}046313

FIG. 5545: PRO83071

FIG. 5546: DNA326730,NM_{—}015953,gen.NM_{—}015953

FIG. 5547: PRO83072

FIG. 5548: DNA326731,XM_{—}027904,gen.XM_{—}027904

FIG. 5549: DNA326732,XM_{—}084026,gen.XM_{—}084026

FIG. 5550: DNA290260,NM_{—}012423,gen.NM_{—}012423

FIG. 5551: PRO70385

FIG. 5552: DNA326733,XM_{—}058991,gen.XM_{—}058991

FIG. 5553: PRO83073

FIG. 5554: DNA326734,NM_{—}017916,gen.NM_{—}017916

FIG. 5555: PRO83074

FIG. 5556: DNA326735,NM_{—}003598,gen.NM_{—}003598

FIG. 5557: PRO83075

FIG. 5558: DNA326736,NM_{—}006666,gen.NM_{—}006666

FIG. 5559: PRO83076

FIG. 5560: DNA326737,XM_{—}114024,gen.XM_{—}114024

FIG. 5561: PRO83077

FIG. 5562: DNA304658,NM_{—}000146,gen.NM_{—}000146

FIG. 5563: PRO71085

FIG. 5564: DNA326738,NM_{—}004324,gen.NM_{—}004324

FIG. 5565: PRO38101

FIG. 5566: DNA326739,NM_{—}006184,gen.NM_{—}006184

FIG. 5567: PRO83078

FIG. 5568: DNA273066,NM_{—}001190,gen.NM_{—}001190

FIG. 5569: PRO61129

FIG. 5570: DNA326740,XM_{—}058987,gen.XM_{—}058987

FIG. 5571: DNA326741,NM_{—}000979,gen.NM_{—}000979

FIG. 5572: PRO83080

FIG. 5573: DNA326742,XM_{—}085935,gen.XM_{—}085935

FIG. 5574: DNA326743,NM_{—}031485,gen.NM_{—}031485

FIG. 5575: PRO61308

FIG. 5576: DNA103239,NM_{—}006801,gen.NM_{—}006801

FIG. 5577: PRO4569

FIG. 5578: DNA326744,XM_{—}046419,gen.XM_{—}046419

FIG. 5579: PRO83082

FIG. 5580: DNA326745,NM_{—}002691,gen.NM_{—}002691

FIG. 5581: PRO83083

FIG. 5582: DNA326746,XM_{—}056286,gen.XM_{—}056286

FIG. 5583: PRO83084

FIG. 5584: DNA326747,XM_{—}058990,gen.XM_{—}058990

FIG. 5585: PRO83085

FIG. 5586: DNA326748,XM_{—}091981,gen.XM_{—}091981

FIG. 5587: PRO83086

FIG. 5588: DNA326749,NM_{—}032712,gen.NM_{—}032712

FIG. 5589: PRO23238

FIG. 5590: DNA83154,NM_{—}001648,gen.NM_{—}001648

FIG. 5591: PRO2109

FIG. 5592: DNA326750,XM_{—}055658,gen.XM_{—}055658

FIG. 5593: DNA269481,NM_{—}001985,gen.NM_{—}001985

FIG. 5594: PRO57901

FIG. 5595: DNA326751,XM_{—}091886,gen.XM_{—}091886

FIG. 5596: PRO83087

FIG. 5597: DNA326752,XM_{—}008830,gen.XM_{—}008830

FIG. 5598: DNA326753,XM_{—}039908,gen.XM_{—}039908

FIG. 5599: PRO83089

FIG. 5600: DNA326754,NM_{—}015629,gen.NM_{—}015629

FIG. 5601: PRO83090

FIG. 5602: DNA326755,XM_{—}050236,gen.XM_{—}050236

FIG. 5603: DNA326756,XM_{—}050589,gen.XM_{—}050589

FIG. 5604: PRO83092

FIG. 5605: DNA326757,XM_{—}117128,gen.XM_{—}117128

FIG. 5606: PRO83093

FIG. 5607: DNA326758,XM_{—}059321,gen.XM_{—}059321

FIG. 5608: DNA326759,NM_{—}003283,gen.NM_{—}003283

FIG. 5609: PRO83095

FIG. 5610A-B: DNA326760,NM_{—}014931,gen.NM_{—}014931

FIG. 5611: PRO83096

FIG. 5612: DNA326761,XM_{—}035919,gen.XM_{—}035919

FIG. 5613: DNA326762,NM_{—}000991,gen.NM_{—}000991

FIG. 5614: PRO83098

FIG. 5615: DNA273346,NM_{—}014501,gen.NM_{—}014501

FIG. 5616: PRO61349

FIG. 5617: DNA326763,NM_{—}013333,gen.NM_{—}013333

FIG. 5618: PRO83099

FIG. 5619: DNA326764,NM_{—}007279,gen.NM_{—}007279

FIG. 5620: PRO83100

FIG. 5621: DNA326765,NM_{—}016202,gen.NM_{—}016202

FIG. 5622: PRO83101

FIG. 5623: DNA326766,XM_{—}034377,gen.XM_{—}034377

FIG. 5624: PRO83102

FIG. 5625: DNA272062,NM_{—}014453,gen.NM_{—}014453

FIG. 5626: PRO60333

FIG. 5627: DNA254548,NM_{—}005762,gen.NM_{—}005762

FIG. 5628: PRO49653

FIG. 5629: DNA326767,XM_{—}085972,gen.XM_{—}085972

FIG. 5630: PRO83103

FIG. 5631: DNA326768,NM_{—}032792,gen.NM_{—}032792

FIG. 5632: PRO83104

FIG. 5633: DNA326769,NM_{—}001009,gen.NM_{—}001009

FIG. 5634: PRO83105

FIG. 5635: DNA326770,XM_{—}058125,gen.XM_{—}058125

FIG. 5636: DNA326771,NM_{—}024691,gen.NM_{—}024691

FIG. 5637: PRO83107

FIG. 5638: DNA297288,NM_{—}021158,gen.NM_{—}021158

FIG. 5639: PRO70810

FIG. 5640: DNA304662,NM_{—}031229,gen.NM_{—}031229

FIG. 5641: PRO71089

FIG. 5642: DNA326772,NM_{—}031228,gen.NM_{—}031228

FIG. 5643: PRO83108

FIG. 5644: DNA326773,XM_{—}097749,gen.XM_{—}097749

FIG. 5645: PRO83109

FIG. 5646: DNA326774,XM_{—}055993,gen.XM_{—}055993

FIG. 5647: DNA326775,XM_{—}009622,gen.XM_{—}009622

FIG. 5648: DNA326776,NM_{—}000801,gen.NM_{—}000801

FIG. 5649: PRO59142

FIG. 5650: DNA326777,NM_{—}054014,gen.NM_{—}054014

FIG. 5651: PRO59142

FIG. 5652: DNA326778,NM_{—}016143,gen.NM_{—}016143

FIG. 5653: PRO83112

FIG. 5654: DNA287270,NM_{—}003091,gen.NM_{—}003091

FIG. 5655: PRO69541

FIG. 5656: DNA326779,NM_{—}052881,gen.NM_{—}052881

FIG. 5657: PRO83113

FIG. 5658: DNA326780,XM_{—}044914,gen.XM_{—}044914

FIG. 5659: PRO83114

FIG. 5660: DNA326781,XM_{—}044915,gen.XM_{—}044915

FIG. 5661: DNA326782,NM_{—}006899,gen.NM_{—}006899

FIG. 5662: PRO83116

FIG. 5663: DNA326783,NM_{—}019609,gen.NM_{—}019609

FIG. 5664: PRO83117

FIG. 5665: DNA326784,NM_{—}021826,gen.NM_{—}021826

FIG. 5666: PRO83118

FIG. 5667: DNA326785,XM_{—}045418,gen.XM_{—}045418

FIG. 5668: DNA287261,NM_{—}017874,gen.NM_{—}017874

FIG. 5669: PRO69533

FIG. 5670: DNA326786,XM_{—}086710,gen.XM_{—}086710

FIG. 5671: DNA326787,XM_{—}045451,gen.XM_{—}045451

FIG. 5672: PRO83121

FIG. 5673: DNA326788,XM_{—}114174,gen.XM_{—}114174

FIG. 5674: DNA326789,XM_{—}045460,gen.XM_{—}045460

FIG. 5675: DNA326790,XM_{—}059268,gen.XM_{—}059268

FIG. 5676A-B: DNA271010,NM_{—}014737,gen.NM_{—}014737

FIG. 5677: PRO59339

FIG. 5678: DNA326791,XM_{—}056035,gen.XM_{—}056035

FIG. 5679: DNA83170,NM_{—}001819,gen.NM_{—}001819

FIG. 5680: PRO2615

FIG. 5681: DNA227348,NM_{—}019095,gen.NM_{—}019095

FIG. 5682: PRO37811

FIG. 5683: DNA326792,NM_{—}003092,gen.NM_{—}003092

FIG. 5684: PRO83125

FIG. 5685: DNA287290,NM_{—}014426,gen.NM_{—}014426

FIG. 5686: PRO69560

FIG. 5687: DNA326793,XM_{—}086701,gen.XM_{—}086701

FIG. 5688: DNA326794,XM_{—}117209,gen.XM_{—}117209

FIG. 5689A-B: DNA326795,XM_{—}046520,gen.XM_{—}046520

FIG. 5690: PRO83128

FIG. 5691: DNA326796,XM_{—}115846,gen.XM_{—}115846

FIG. 5692: PRO83129

FIG. 5693: DNA326797,NM_{—}080820,gen.NM_{—}080820

FIG. 5694: PRO83130

FIG. 5695: DNA326798,XM_{—}086715,gen.XM_{—}086715

FIG. 5696: DNA326799,XM_{—}092760,gen.XM_{—}092760

FIG. 5697: PRO83132

FIG. 5698: DNA326800,NM_{—}012255,gen.NM_{—}012255

FIG. 5699: PRO83133

FIG. 5700: DNA326801,XM_{—}012970,gen.XM_{—}012970

FIG. 5701: DNA326802,XM_{—}042765,gen.XM_{—}042765

FIG. 5702: PRO83135

FIG. 5703: DNA150548,NM_{—}001247,gen.NM_{—}001247

FIG. 5704: PRO12324

FIG. 5705A-B: DNA326803,XM_{—}009436,gen.XM_{—}009436

FIG. 5706: DNA326804,XM_{—}114178,gen.XM_{—}114178

FIG. 5707: PRO83137

FIG. 5708: DNA326805,XM_{—}046160,gen.XM_{—}046160

FIG. 5709: PRO83138

FIG. 5710: DNA326806,XM_{—}046179,gen.XM_{—}046179

FIG. 5711: PRO83139

FIG. 5712: DNA326807,XM_{—}086745,gen.XM_{—}086745

FIG. 5713: DNA326808,NM_{—}138578,gen.NM_{—}138578

FIG. 5714: PRO83141

FIG. 5715: DNA326809,NM_{—}012112,gen.NM_{—}012112

FIG. 5716: PRO83142

FIG. 5717: DNA326810,XM_{—}086736,gen.XM_{—}086736

FIG. 5718: PRO83143

FIG. 5719: DNA326811,NM_{—}030815,gen.NM_{—}030815

FIG. 5720: PRO83144

FIG. 5721A-B: DNA150767,NM_{—}014742,gen.NM_{—}014742

FIG. 5722: PRO12460

FIG. 5723A-B: DNA326812,XM_{—}047007,gen.XM_{—}047007

FIG. 5724: PRO83145

FIG. 5725A-B: DNA326813,XM_{—}047011,gen.XM_{—}047011

FIG. 5726: PRO83146

FIG. 5727A-B: DNA326814,XM_{—}047018,gen.XM_{—}047018

FIG. 5728: DNA326815,XM_{—}009450,gen.XM_{—}009450

FIG. 5729: DNA326816,NM_{—}033197,gen.NM_{—}033197

FIG. 5730: PRO83149

FIG. 5731: DNA326817,XM_{—}097772,gen.XM_{—}097772

FIG. 5732: PRO83150

FIG. 5733: DNA326818,NM_{—}016732,gen.NM_{—}016732

FIG. 5734: DNA97298,NM_{—}003908,gen.NM_{—}003908

FIG. 5735: PRO3645

FIG. 5736: DNA326819,NM_{—}000687,gen.NM_{—}000687

FIG. 5737: PRO83152

FIG. 5738: DNA273517,NM_{—}000178,gen.NM_{—}000178

FIG. 5739: PRO61498

FIG. 5740: DNA326820,NM_{—}018217,gen.NM_{—}018217

FIG. 5741: PRO83153

FIG. 5742: DNA326821,NM_{—}002212,gen.NM_{—}002212

FIG. 5743: PRO60945

FIG. 5744A-C: DNA326822,NM_{—}07186,gen.NM_{—}007186

FIG. 5745: DNA226758,NM_{—}015966,gen.NM_{—}015966

FIG. 5746: PRO37221

FIG. 5747: DNA194701,NM_{—}03915,gen.NM_{—}003915

FIG. 5748: PRO24002

FIG. 5749: DNA326823,XM_{—}113380,gen.XM_{—}113380

FIG. 5750: DNA326824,NM_{—}016558,gen.NM_{—}016558

FIG. 5751: PRO83155

FIG. 5752: DNA326825,NM_{—}015511,gen.NM_{—}015511

FIG. 5753: PRO83156

FIG. 5754: DNA326826,XM_{—}009501,gen.XM_{—}009501

FIG. 5755: PRO83157

FIG. 5756: DNA326827,XM_{—}057236,gen.XM_{—}057236

FIG. 5757: DNA326828,NM_{—}024918,gen.NM_{—}024918

FIG. 5758: PRO83159

FIG. 5759: DNA326829,XM_{—}009642,gen.XM_{—}009642

FIG. 5760: DNA194807,NM_{—}006698,gen.NM_{—}006698

FIG. 5761: PRO24077

FIG. 5762: DNA326830,XM_{—}009686,gen.XM_{—}009686

FIG. 5763: DNA326831,NM_{—}030877,gen.NM_{—}030877

FIG. 5764: PRO83161

FIG. 5765: DNA326832,XM_{—}028806,gen.XM_{—}028806

FIG. 5766A-B: DNA326833,XM_{—}028810,gen.XM_{—}028810

FIG. 5767: PRO83163

FIG. 5768: DNA326834,XM_{—}012931,gen.XM_{—}012931

FIG. 5769: DNA326835,NM_{—}024855,gen.NM_{—}024855

FIG. 5770: PRO83165

FIG. 5771A-B: DNA227472,NM_{—}002660,gen.NM_{—}002660

FIG. 5772: PRO37935

FIG. 5773: DNA326836,XM_{—}097727,gen.XM_{—}097727

FIG. 5774: DNA103525,NM_{—}002466,gen.NM_{—}002466

FIG. 5775: PRO4852

FIG. 5776: DNA326837,XM_{—}029810,gen.XM_{—}029810

FIG. 5777: PRO83167

FIG. 5778: DNA326838,XM_{—}29822,gen.XM_{—}029822

FIG. 5779: DNA326839,NM_{—}002638,gen.NM_{—}002638

FIG. 5780: PRO2065

FIG. 5781: DNA326840,NM_{—}03064,gen.NM_{—}003064

FIG. 5782: PRO1720

FIG. 5783: DNA326841,NM_{—}015937,gen.NM_{—}015937

FIG. 5784: PRO83169

FIG. 5785: DNA273320,NM_{—}007019,gen.NM_{—}007019

FIG. 5786: PRO61327

FIG. 5787: DNA326842,NM_{—}033421,gen.NM_{—}033421

FIG. 5788: PRO83170

FIG. 5789: DNA88569,NM_{—}006227,gen.NM_{—}006227

FIG. 5790: PRO2420

FIG. 5791: DNA88239,NM_{—}004994,gen.NM_{—}004994

FIG. 5792: PRO2711

FIG. 5793: DNA326843,XM_{—}057374,gen.XM_{—}057374

FIG. 5794: DNA326844,XM_{—}114163,gen.XM_{—}114163

FIG. 5795A-B: DNA326845,XM_{—}097731,gen.XM_{—}097731

FIG. 5796A-B: DNA326846,XM_{—}030044,gen.XM_{—}030044

FIG. 5797: PRO83174

FIG. 5798: DNA326847,NM_{—}017895,gen.NM_{—}017895

FIG. 5799: PRO83175

FIG. 5800: DNA326848,XM_{—}097713,gen.XM_{—}097713

FIG. 5801: PRO83176

FIG. 5802: DNA326849,NM_{—}005985,gen.NM_{—}005985

FIG. 5803: PRO83177

FIG. 5804: DNA326850,NM_{—}003349,gen.NM_{—}003349

FIG. 5805: PRO83178

FIG. 5806: DNA326851,NM_{—}022442,gen.NM_{—}022442

FIG. 5807: PRO83179

FIG. 5808: DNA326852,NM_{—}005194,gen.NM_{—}005194

FIG. 5809: DNA326853,NM_{—}002827,gen.NM_{—}002827

FIG. 5810: PRO38066

FIG. 5811: DNA326854,NM_{—}003859,gen.NM_{—}003859

FIG. 5812: PRO83180

FIG. 5813: DNA326855,XM_{—}114165,gen.XM_{—}114165

FIG. 5814: DNA269526,NM_{—}001324,gen.NM_{—}001324

FIG. 5815: PRO57942

FIG. 5816: DNA326856,XM_{—}009549,gen.XM_{—}009549

FIG. 5817: PRO83182

FIG. 5818: DNA326857,XM_{—}030621,gen.XM_{—}030621

FIG. 5819: DNA326858,XM_{—}086648,gen.XM_{—}086648

FIG. 5820: PRO83183

FIG. 5821: DNA326859,XM_{—}009672,gen.XM_{—}009672

FIG. 5822: PRO83184

FIG. 5823A-B: DNA326860,XM_{—}009671,gen.XM_{—}009671

FIG. 5824: DNA326861,NM_{—}004738,gen.NM_{—}004738

FIG. 5825: PRO983

FIG. 5826: DNA326862,NM_{—}016592,gen.NM_{—}016592

FIG. 5827: PRO83185

FIG. 5828: DNA326863,NM_{—}080425,gen.NM_{—}080425

FIG. 5829: PRO83186

FIG. 5830: DNA304670,NM_{—}000516,gen.NM_{—}000516

FIG. 5831: PRO71097

FIG. 5832: DNA326864,NM_{—}080426,gen.NM_{—}080426

FIG. 5833: PRO83187

FIG. 5834: DNA326865,XM_{—}030699,gen.XM_{—}030699

FIG. 5835: PRO83188

FIG. 5836: DNA188229,NM_{—}000114,gen.NM_{—}000114

FIG. 5837: PRO21728

FIG. 5838: DNA326866,NM_{—}002792,gen.NM_{—}002792

FIG. 5839: PRO83189

FIG. 5840A-B: DNA326867,XM_{—}037202,gen.XM_{—}037202

FIG. 5841: PRO83190

FIG. 5842: DNA326868,XM_{—}037206,gen.XM_{—}037206

FIG. 5843: PRO83191

FIG. 5844: DNA103486,NM_{—}007002,gen.NM_{—}007002

FIG. 5845: PRO4813

FIG. 5846A-D: DNA326869,XM_{—}037217,gen.XM_{—}037217

FIG. 5847: DNA326870,NM_{—}001024,gen.NM_{—}001024

FIG. 5848: PRO83193

FIG. 5849: DNA326871,NM_{—}018270,gen.NM_{—}018270

FIG. 5850: PRO83194

FIG. 5851: DNA326872,XM_{—}028783,gen.XM_{—}028783

FIG. 5852: PRO83195

FIG. 5853: DNA326873,NM_{—}001853,gen.NM_{—}001853

FIG. 5854: PRO83196

FIG. 5855: DNA326874,NM_{—}080796,gen.NM_{—}080796

FIG. 5856: PRO83197

FIG. 5857: DNA326875,NM_{—}022105,gen.NM_{—}022105

FIG. 5858: PRO83198

FIG. 5859: DNA326876,NM_{—}080797,gen.NM_{—}080797

FIG. 5860: PRO83199

FIG. 5861: DNA326877,NM_{—}018209,gen.NM_{—}018209

FIG. 5862: PRO83200

FIG. 5863A-C: DNA326878,XM_{—}028834,gen.XM_{—}028834

FIG. 5864: PRO83201

FIG. 5865: DNA326879,NM_{—}024299,gen.NM_{—}024299

FIG. 5866: PRO83202

FIG. 5867A-C: DNA326880,XM_{—}028918,gen.XM_{—}028918

FIG. 5868: PRO83203

FIG. 5869: DNA326881,NM_{—}032527,gen.NM_{—}032527

FIG. 5870: PRO83204

FIG. 5871A-B: DNA326882,XM_{—}028966,gen.XM_{—}028966

FIG. 5872: PRO83205

FIG. 5873: DNA269746,NM_{—}012469,gen.NM_{—}012469

FIG. 5874: PRO58155

FIG. 5875: DNA326883,XM_{—}114154,gen.XM_{—}114154

FIG. 5876: DNA326884,XM_{—}072173,gen.XM_{—}072173

FIG. 5877: DNA326885,XM_{—}086759,gen.XM_{—}086759

FIG. 5878: DNA326886,XM_{—}086760,gen.XM_{—}086760

FIG. 5879: DNA326887,NM_{—}021219,gen.NM_{—}021219

FIG. 5880: PRO28687

FIG. 5881: DNA188732,NM_{—}000484,gen.NM_{—}000484

FIG. 5882: PRO25302

FIG. 5883: DNA326888,NM_{—}016940,gen.NM_{—}016940

FIG. 5884: PRO83210

FIG. 5885: DNA254572,NM_{—}006585,gen.NM_{—}006585

FIG. 5886: PRO49675

FIG. 5887: DNA326889,NM_{—}105806,gen.NM_{—}005806

FIG. 5888: PRO83211

FIG. 5889: DNA326890,XM_{—}114185,gen.XM_{—}114185

FIG. 5890: DNA254994,NM_{—}017613,gen.NM_{—}017613

FIG. 5891: PRO50083

FIG. 5892: DNA274129,NM_{—}001697,gen.NM_{—}001697

FIG. 5893: PRO62065

FIG. 5894: DNA326891,NM_{—}001757,gen.NM_{—}001757

FIG. 5895: PRO83212

FIG. 5896A-C: DNA151898,NM_{—}003316,gen.NM_{—}003316

FIG. 5897: PRO12135

FIG. 5898: DNA326892,NM_{—}003720,gen.NM_{—}003720

FIG. 5899: PRO83213

FIG. 5900: DNA326893,NM_{—}002606,gen.NM_{—}002606

FIG. 5901: PRO83214

FIG. 5902: DNA326894,XM_{—}033015,gen.XM_{—}033015

FIG. 5903: DNA326895,XM_{—}033016,gen.XM_{—}033016

FIG. 5904: PRO59669

FIG. 5905: DNA326896,NM_{—}003681,gen.NM_{—}003681

FIG. 5906: PRO69486

FIG. 5907: DNA326897,XM_{—}035999,gen.XM_{—}035999

FIG. 5908: DNA326898,NM_{—}020132,gen.NM_{—}020132

FIG. 5909: PRO83217

FIG. 5910: DNA326899,XM_{—}036011,gen.XM_{—}036011

FIG. 5911: DNA326900,NM_{—}013369,gen.NM_{—}013369

FIG. 5912: PRO83219

FIG. 5913: DNA326901,XM_{—}036042,gen.XM_{—}036042

FIG. 5914: DNA326902,XM_{—}086770,gen.XM_{—}086770

FIG. 5915: DNA326903,NM_{—}004928,gen.NM_{—}004928

FIG. 5916: PRO83222

FIG. 5917: DNA326904,XM_{—}036087,gen.XM_{—}036087

FIG. 5918: PRO83223

FIG. 5919: DNA326905,XM_{—}009805,gen.XM_{—}009805

FIG. 5920: PRO83224

FIG. 5921: DNA226409,NM_{—}004339,gen.NM_{—}004339

FIG. 5922: PRO36872

FIG. 5923: DNA326906,XM_{—}036107,gen.XM_{—}036107

FIG. 5924A-B: DNA326907,XM_{—}036175,gen.XM_{—}036175

FIG. 5925: DNA326908,XM_{—}097817,gen.XM_{—}097817

FIG. 5926A-B: DNA326909,XM_{—}054566,gen.XM_{—}054566

FIG. 5927: DNA326910,XM_{—}036755,gen.XM_{—}036755

FIG. 5928: DNA326911,XM_{—}086773,gen.XM_{—}086773

FIG. 5929: DNA326912,XM_{—}097807,gen.XM_{—}097807

FIG. 5930: DNA326913,XM_{—}086777,gen.XM_{—}086777

FIG. 5931: DNA326914,NM_{—}002340,gen.NM_{—}002340

FIG. 5932: PRO83233

FIG. 5933A-B: DNA326915,NM_{—}003906,gen.NM_{—}003906

FIG. 5934: PRO83234

FIG. 5935: DNA226617,NM_{—}006272,gen.NM_{—}006272

FIG. 5936: PRO37080

FIG. 5937: DNA326916,NM_{—}033070,gen.NM_{—}033070

FIG. 5938: PRO83235

FIG. 5939: DNA255046,NM_{—}017829,gen.NM_{—}017829

FIG. 5940: PRO50134

FIG. 5941: DNA326917,NM_{—}001696,gen.NM_{—}001696

FIG. 5942: PRO83236

FIG. 5943A-B: DNA326918,XM_{—}032996,gen.XM_{—}032996

FIG. 5944: PRO83237

FIG. 5945: DNA326919,XM_{—}167538,gen.XM_{—}167538

FIG. 5946: DNA326920,XM_{—}033090,gen.XM_{—}033090

FIG. 5947: DNA225954,NM_{—}000407,gen.NM_{—}000407

FIG. 5948: PRO36417

FIG. 5949: DNA326921,XM_{—}058918,gen.XM_{—}058918

FIG. 5950: DNA326922,XM_{—}097833,gen.XM_{—}097833

FIG. 5951: DNA326923,NM_{—}024627,gen.NM_{—}024627

FIG. 5952: PRO83242

FIG. 5953: DNA326924,XM_{—}086809,gen.XM_{—}086809

FIG. 5954: DNA326925,NM_{—}006440,gen.NM_{—}006440

FIG. 5955: PRO83244

FIG. 5956: DNA226561,NM_{—}000754,gen.NM_{—}000754

FIG. 5957: PRO37024

FIG. 5958: DNA326926,NM_{—}007310,gen.NM_{—}007310

FIG. 5959: PRO83245

FIG. 5960A-B: DNA326927,XM_{—}033813,gen.XM_{—}033813

FIG. 5961: DNA326928,NM_{—}022727,gen.NM_{—}022727

FIG. 5962: PRO83247

FIG. 5963: DNA326929,XM_{—}086805,gen.XM_{—}086805

FIG. 5964: DNA326930,XM_{—}086873,gen.XM_{—}086873

FIG. 5965: DNA257549,NM_{—}030573,gen.NM_{—}030573

FIG. 5966: PRO52119

FIG. 5967: DNA326931,XM_{—}096155,gen.XM_{—}096155

FIG. 5968: DNA326932,XM_{—}096156,gen.XM_{—}096156

FIG. 5969A-B: DNA326933,XM_{—}036937,gen.XM_{—}036937

FIG. 5970: PRO83252

FIG. 5971: DNA326934,XM_{—}097886,gen.XM_{—}097886

FIG. 5972: PRO83253

FIG. 5973: DNA304835,NM_{—}022044,gen.NM_{—}022044

FIG. 5974: PRO71242

FIG. 5975: DNA326935,NM_{—}006115,gen.NM_{—}006115

FIG. 5976: PRO37012

FIG. 5977: DNA326936,XM_{—}037682,gen.XM_{—}037682

FIG. 5978: PRO83254

FIG. 5979: DNA326937,NM_{—}002415,gen.NM_{—}002415

FIG. 5980: PRO83255

FIG. 5981A-B: DNA326938,XM_{—}037797,gen.XM_{—}037797

FIG. 5982: PRO83256

FIG. 5983: DNA326939,NM_{—}004175,gen.NM_{—}004175

FIG. 5984: PRO83257

FIG. 5985: DNA326940,XM_{—}086821,gen.XM_{—}086821

FIG. 5986: DNA326941,XM_{—}092888,gen.XM_{—}092888

FIG. 5987: DNA326942,NM_{—}005080,gen.NM_{—}005080

FIG. 5988: PRO83260

FIG. 5989: DNA269830,NM_{—}005243,gen.NM_{—}005243

FIG. 5990: PRO58232

FIG. 5991: DNA326943,NM_{—}006478,gen.NM_{—}006478

FIG. 5992: PRO83261

FIG. 5993A-B: DNA326944,XM_{—}037945,gen.XM_{—}037945

FIG. 5994: DNA103462,NM_{—}000268,gen.NM_{—}000268

FIG. 5995: PRO4789

FIG. 5996: DNA326945,NM_{—}032204,gen.NM_{—}032204

FIG. 5997: PRO83263

FIG. 5998: DNA326946,XM_{—}066291,gen.XM_{—}066291

FIG. 5999: DNA326947,NM_{—}005877,gen.NM_{—}005877

FIG. 6000: PRO62328

FIG. 6001: DNA326948,NM_{—}016498,gen.NM_{—}016498

FIG. 6002: PRO83265

FIG. 6003: DNA254141,NM_{—}014303,gen.NM_{—}014303

FIG. 6004: PRO49256

FIG. 6005A-B: DNA151882,NM_{—}014941,gen.NM_{—}014941

FIG. 6006: PRO12134

FIG. 6007: DNA326949,NM_{—}006932,gen.NM_{—}006932

FIG. 6008: PRO83266

FIG. 6009: DNA326950,NM_{—}134269,gen.NM_{—}134269

FIG. 6010: PRO83267

FIG. 6011: DNA270697,NM_{—}004147,gen.NM_{—}004147

FIG. 6012: PRO59061

FIG. 6013: DNA326951,XM_{—}059335,gen.XM_{—}059335

FIG. 6014: DNA326952,XM_{—}018539,gen.XM_{—}018539

FIG. 6015: DNA326953,NM_{—}014306,gen.NM_{—}014306

FIG. 6016: PRO83270

FIG. 6017: DNA326954,NM_{—}012179,gen.NM_{—}012179

FIG. 6018: PRO83271

FIG. 6019A-B: DNA326955,XM_{—}038584,gen.XM_{—}038584

FIG. 6020: DNA151752,NM_{—}002133,gen.NM_{—}002133

FIG. 6021: PRO12886

FIG. 6022: DNA326956,XM_{—}009947,gen.XM_{—}009947

FIG. 6023: PRO12845

FIG. 6024: DNA326957,XM_{—}114209,gen.XM_{—}114209

FIG. 6025A-B: DNA326958,NM_{—}002473,gen.NM_{—}002473

FIG. 6026: PRO83273

FIG. 6027: DNA188740,NM_{—}003753,gen.NM_{—}003753

FIG. 6028: PRO22481

FIG. 6029: DNA326959,NM_{—}021126,gen.NM_{—}021126

FIG. 6030: PRO70331

FIG. 6031: DNA326960,XM_{—}009967,gen.XM_{—}009967

FIG. 6032: DNA326961,NM_{—}013365,gen.NM_{—}013365

FIG. 6033: PRO83274

FIG. 6034: DNA290259,NM_{—}018957,gen.NM_{—}018957

FIG. 6035: PRO70383

FIG. 6036: DNA326962,NM_{—}020315,gen.NM_{—}020315

FIG. 6037: PRO83275

FIG. 6038: DNA304719,NM_{—}002305,gen.NM_{—}002305

FIG. 6039: PRO71145

FIG. 6040: DNA326963,NM_{—}007032,gen.NM_{—}007032

FIG. 6041: PRO83276

FIG. 6042: DNA326964,XM_{—}009973,gen.XM_{—}009973

FIG. 6043: DNA326965,XM_{—}086830,gen.XM_{—}086830

FIG. 6044: PRO83278

FIG. 6045: DNA254240,NM_{—}016091,gen.NM_{—}016091

FIG. 6046: PRO49352

FIG. 6047A-B: DNA326966,XM_{—}039236,gen.XM_{—}039236

FIG. 6048: PRO83279

FIG. 6049: DNA326967,NM_{—}006941,gen.NM_{—}006941

FIG. 6050: PRO83280

FIG. 6051: DNA326968,XM_{—}039248,gen.XM_{—}039248

FIG. 6052: DNA326969,NM_{—}012323,gen.NM_{—}012323

FIG. 6053: PRO83282

FIG. 6054: DNA326970,NM_{—}012264,gen.NM_{—}012264

FIG. 6055: PRO 12490

FIG. 6056: DNA326971,NM_{—}015373,gen.NM_{—}015373

FIG. 6057: PRO83283

FIG. 6058: DNA326972,NM_{—}020243,gen.NM_{—}020243

FIG. 6059: PRO23231

FIG. 6060: DNA326973,XM_{—}039339,gen.XM_{—}039339

FIG. 6061: DNA326974,NM_{—}000967,gen.NM_{—}000967

FIG. 6062: PRO83285

FIG. 6063: DNA326975,XM_{—}010000,gen.XM_{—}010000

FIG. 6064: DNA326976,XM_{—}010002,gen.XM_{—}010002

FIG. 6065: DNA326977,XM_{—}039372,gen.XM_{—}039372

FIG. 6066: DNA326978,XM_{—}013010,gen.XM_{—}013010

FIG. 6067: PRO83288

FIG. 6068: DNA254165,NM_{—}000026,gen.NM_{—}000026

FIG. 6069: PRO49278

FIG. 6070: DNA326979,NM_{—}003932,gen.NM_{—}003932

FIG. 6071: PRO4586

FIG. 6072: DNA326980,NM_{—}014248,gen.NM_{—}014248

FIG. 6073: PRO83289

FIG. 6074: DNA326981,XM_{—}086844,gen.XM_{—}086844

FIG. 6075: DNA219225,NM_{—}002883,gen.NM_{—}002883

FIG. 6076: PRO34531

FIG. 6077: DNA326982,NM_{—}003216,gen.NM_{—}003216

FIG. 6078: PRO83291

FIG. 6079: DNA270954,NM_{—}001098,gen.NM_{—}001098

FIG. 6080: PRO59285

FIG. 6081: DNA326983,NM_{—}001469,gen.NM_{—}001469

FIG. 6082: PRO4872

FIG. 6083: DNA326984,NM_{—}005008,gen.NM_{—}005008

FIG. 6084: PRO83292

FIG. 6085A-B: DNA326985,NM_{—}004599,gen.NM_{—}004599

FIG. 6086: PRO83293

FIG. 6087A-B: DNA326986,XM_{—}010024,gen.XM_{—}010024

FIG. 6088: DNA326987,XM_{—}040066,gen.XM_{—}040066

FIG. 6089: DNA326988,XM_{—}013015,gen.XM_{—}013015

FIG. 6090A-B: DNA326989,XM_{—}084084,gen.XM_{—}084084

FIG. 6091: DNA326990,XM_{—}040095,gen.XM_{—}040095

FIG. 6092: PRO83297

FIG. 6093: DNA326991,XM_{—}086875,gen.XM_{—}086875

FIG. 6094: DNA326992,XM_{—}010029,gen.XM_{—}010029

FIG. 6095: DNA326993,NM_{—}007311,gen.NM_{—}007311

FIG. 6096: PRO83300

FIG. 6097: DNA326994,NM_{—}015140,gen.NM_{—}015140

FIG. 6098: PRO83301

FIG. 6099: DNA326995,XM_{—}043614,gen.XM_{—}043614

FIG. 6100: PRO83302

FIG. 6101: DNA256070,NM_{—}022141,gen.NM_{—}022141

FIG. 6102: PRO51119

FIG. 6103: DNA326996,XM_{—}010040,gen.XM_{—}010040

FIG. 6104: DNA237931,NM_{—}005036,gen.NM_{—}005036

FIG. 6105: PRO39030

FIG. 6106A-B: DNA326997,XM_{—}027143,gen.XM_{—}027143

FIG. 6107: PRO83304

FIG. 6108A-B: DNA326998,XM_{—}010055,gen.XM_{—}010055

FIG. 6109: DNA326999,NM_{—}025204,gen.NM_{—}025204

FIG. 6110: PRO83306

FIG. 6111: DNA327000,XM_{—}041248,gen.XM_{—}041248

FIG. 6112: PRO83307

FIG. 6113: DNA327001,XM_{—}092966,gen.XM_{—}092966

FIG. 6114: DNA327002,XM_{—}037468,gen.XM_{—}037468

FIG. 6115: PRO83309

FIG. 6116: DNA327003,XM_{—}037474,gen.XM_{—}037474

FIG. 6117: PRO83310

FIG. 6118: DNA327004,XM_{—}013029,gen.XM_{—}013029

FIG. 6119: DNA327005,XM_{—}114724,gen.XM_{—}114724

FIG. 6120: PRO83312

FIG. 6121: DNA327006,XM_{—}115924,gen.XM_{—}115924

FIG. 6122: DNA327007,XM_{—}113585,gen.XM_{—}113585

FIG. 6123A-C: DNA327008,XM_{—}035465,gen.XM_{—}035465

FIG. 6124: DNA327009,NM_{—}002414,gen.NM_{—}002414

FIG. 6125: PRO2373

FIG. 6126: DNA269793,NM_{—}005333,gen.NM_{—}005333

FIG. 6127: PRO58198

FIG. 6128: DNA327010,XM_{—}088747,gen.XM_{—}088747

FIG. 6129: PRO83316

FIG. 6130: DNA327011,XM_{—}114720,gen.XM_{—}114720

FIG. 6131: DNA327012,XM_{—}115886,gen.XM_{—}115886

FIG. 6132: DNA327013,XM_{—}010272,gen.XM_{—}010272

FIG. 6133: PRO83319

FIG. 6134: DNA327014,NM_{—}006746,gen.NM_{—}006746

FIG. 6135: PRO83320

FIG. 6136: DNA327015,XM_{—}115890,gen.XM_{—}115890

FIG. 6137: PRO83321

FIG. 6138: DNA327016,NM_{—}000284,gen.NM_{—}000284

FIG. 6139: PRO59441

FIG. 6140: DNA327017,NM_{—}004595,gen.NM_{—}004595

FIG. 6141: PRO61744

FIG. 6142: DNA327018,XM_{—}166078,gen.XM_{—}166078

FIG. 6143: DNA327019,NM_{—}001415,gen.NM_{—}001415

FIG. 6144: PRO83323

FIG. 6145: DNA327020,XM_{—}013086,gen.XM_{—}013086

FIG. 6146: DNA327021,XM_{—}060030,gen.XM_{—}060030

FIG. 6147: DNA227689,NM_{—}002364,gen.NM_{—}002364

FIG. 6148: PRO38152

FIG. 6149: DNA274829,NM_{—}003662,gen.NM_{—}003662

FIG. 6150: PRO62588

FIG. 6151: DNA327022,XM_{—}088619,gen.XM_{—}088619

FIG. 6152: DNA327023,XM_{—}088622,gen.XM_{—}088622

FIG. 6153A-B: DNA327024,XM_{—}084288,gen.XM_{—}084288

FIG. 6154: PRO59168

FIG. 6155: DNA327025,XM_{—}054221,gen.XM_{—}054221

FIG. 6156: PRO83328

FIG. 6157: DNA327026,XM_{—}018019,gen.XM_{—}018019

FIG. 6158: DNA327027,XM_{—}088665,gen.XM_{—}088665

FIG. 6159: DNA327028,NM_{—}005300,gen.NM_{—}005300

FIG. 6160: PRO37083

FIG. 6161: DNA327029,XM_{—}018241,gen.XM_{—}018241

FIG. 6162: PRO83331

FIG. 6163: DNA327030,NM_{—}014138,gen.NM_{—}014138

FIG. 6164: PRO83332

FIG. 6165: DNA32703 1,NM_{—}005676,gen.NM_{—}005676

FIG. 6166: PRO83333

FIG. 6167: DNA327032,NM_{—}003334,gen.NM_{—}003334

FIG. 6168: PRO83334

FIG. 6169: DNA327033,XM_{—}010378,gen.XM_{—}010378

FIG. 6170: DNA327034,XM_{—}033884,gen.XM_{—}033884

FIG. 6171: PRO83335

FIG. 6172: DNA327035,XM_{—}033878,gen.XM_{—}033878

FIG. 6173: DNA327036,XM_{—}033862,gen.XM_{—}033862

FIG. 6174: DNA327037,NM_{—}004182,gen.NM_{—}004182

FIG. 6175: PRO83337

FIG. 6176: DNA327038,XM_{—}047032,gen.XM_{—}047032

FIG. 6177: DNA327039,XM_{—}047024,gen.XM_{—}047024

FIG. 6178: PRO83339

FIG. 6179: DNA327040,NM_{—}017883,gen.NM_{—}017883

FIG. 6180: PRO83340

FIG. 6181: DNA238039,NM_{—}005710,gen.NM_{—}005710

FIG. 6182: PRO39127

FIG. 6183: DNA327041,XM_{—}054098,gen.XM_{—}054098

FIG. 6184: PRO83341

FIG. 6185: DNA327042,NM_{—}002668,gen.NM_{—}002668

FIG. 6186: PRO34584

FIG. 6187: DNA271580,NM_{—}014008,gen.NM_{—}014008

FIG. 6188: PRO59868

FIG. 6189A-B: DNA327043,XM_{—}032930,gen.XM_{—}032930

FIG. 6190: DNA273992,NM_{—}004493,gen.NM_{—}004493

FIG. 6191: PRO61938

FIG. 6192A-B: DNA327044,XM_{—}050403,gen.XM_{—}050403

FIG. 6193: PRO83343

FIG. 6194: DNA327045,XM_{—}029187,gen.XM_{—}029187

FIG. 6195: PRO83344

FIG. 6196: DNA327046,XM_{—}013060,gen.XM_{—}013060

FIG. 6197: DNA227943,NM_{—}006787,gen.NM_{—}006787

FIG. 6198: PRO38406

FIG. 6199: DNA327047,NM_{—}014481,gen.NM_{—}014481

FIG. 6200: PRO83345

FIG. 6201: DNA327048,XM_{—}034935,gen.XM_{—}034935

FIG. 6202: PRO83346

FIG. 6203: DNA327049,XM_{—}084287,gen.XM_{—}084287

FIG. 6204: DNA327050,NM_{—}007268,gen.NM_{—}007268

FIG. 6205: PRO34043

FIG. 6206: DNA327051,XM_{—}015516,gen.XM_{—}015516

FIG. 6207A-B: DNA027052,XM_{—}013042,gen.XM_{—}013042

FIG. 6208: PRO83349

FIG. 6209: DNA327053,XM_{—}088630,gen.XM_{—}088630

FIG. 6210: DNA327054,NM_{—}031206,gen.NM_{—}031206

FIG. 6211: PRO83351

FIG. 6212: DNA327055,XM_{—}093050,gen.XM_{—}093050

FIG. 6213: PRO83352

FIG. 6214A-B: DNA225721,NM_{—}018977,gen.NM_{—}018977

FIG. 6215: PRO36184

FIG. 6216: DNA327056,XM_{—}010141,gen.XM_{—}010141

FIG. 6217: PRO38021

FIG. 6218: DNA327057,XM_{—}088689,gen.XM_{—}088689

FIG. 6219: PRO83353

FIG. 6220: DNA327058,XM_{—}088688,gen.XM_{—}088688

FIG. 6221: PRO83354

FIG. 6222: DNA327059,NM_{—}018486,gen.NM_{—}018486

FIG. 6223: PRO83355

FIG. 6224: DNA327060,NM_{—}001007,gen.NM_{—}001007

FIG. 6225: PRO42022

FIG. 6226: DNA327061,XM_{—}093130,gen.XM_{—}093130

FIG. 6227: DNA327062,XM_{—}084296,gen.XM_{—}084296

FIG. 6228: DNA327063,XM_{—}093241,gen.XM_{—}093241

FIG. 6229: DNA327064,XM_{—}084283,gen.XM_{—}084283

FIG. 6230: DNA273254,NM_{—}000291,gen.NM_{—}000291

FIG. 6231: PRO61271

FIG. 6232: DNA327065,XM_{—}018142,gen.XM_{—}018142

FIG. 6233: DNA327066,XM_{—}030373,gen.XM_{—}030373

FIG. 6234: PRO83360

FIG. 6235: DNA327067,XM_{—}165533,gen.XM_{—}165533

FIG. 6236: PRO83361

FIG. 6237: DNA327068,XM_{—}051476,gen.XM_{—}051476

FIG. 6238: DNA327069,XM_{—}051471,gen.XM_{—}051471

FIG. 6239: DNA270496,NM_{—}001325,gen.NM_{—}001325

FIG. 6240: PRO58875

FIG. 6241: DNA327070,XM_{—}033147,gen.XM_{—}033147

FIG. 6242: DNA327071,NM_{—}004085,gen.NM_{—}004085

FIG. 6243: PRO59022

FIG. 6244: DNA327072,NM_{—}021029,gen.NM_{—}021029

FIG. 6245: PRO10723

FIG. 6246: DNA327073,NM_{—}012286,gen.NM_{—}012286

FIG. 6247: PRO83365

FIG. 6248: DNA327074,NM_{—}024863,gen.NM_{—}024863

FIG. 6249: PRO83366

FIG. 6250: DNA327075,XM_{—}043643,gen.XM_{—}043643

FIG. 6251: DNA327076,NM_{—}052936,gen.NM_{—}052936

FIG. 6252: PRO83368

FIG. 6253: DNA327077,XM_{—}088710,gen.XM_{—}088710

FIG. 6254: PRO83369

FIG. 6255: DNA327078,XM_{—}166081,gen.XM_{—}166081

FIG. 6256: DNA327079,XM_{—}096303,gen.XM_{—}096303

FIG. 6257: DNA254785,NM_{—}032227,gen.NM_{—}032227

FIG. 6258: PRO49883

FIG. 6259: DNA327080,XM_{—}115923,gen.XM_{—}115923

FIG. 6260: PRO83372

FIG. 6261: DNA327081,XM_{—}066900,gen.XM_{—}066900

FIG. 6262: PRO83373

FIG. 6263: DNA327082,XM_{—}104983,gen.XM_{—}104983

FIG. 6264: PRO83374

FIG. 6265: DNA327083,XM_{—}088736,gen.XM_{—}088736

FIG. 6266: PRO83375

FIG. 6267: DNA327084,XM_{—}088738,gen.XM_{—}088738

FIG. 6268: DNA327085,XM_{—}088739,gen.XM_{—}088739

FIG. 6269: DNA327086,XM_{—}010117,gen.XM_{—}010117

FIG. 6270A-B: DNA76504,NM_{—}001560,gen.NM_{—}001560

FIG. 6271: PRO2537

FIG. 6272: DNA227181,NM_{—}006667,gen.NM_{—}006667

FIG. 6273: PRO37644

FIG. 6274: DNA327087,XM_{—}010362,gen.XM_{—}010362

FIG. 6275: DNA327088,XM_{—}016125,gen.XM_{—}016125

FIG. 6276: DNA327089,NM_{—}015129,gen.NM_{—}015129

FIG. 6277: PRO83381

FIG. 6278: DNA327090,NM_{—}001000,gen.NM_{—}001000

FIG. 6279: PRO10935

FIG. 6280: DNA327091,XM_{—}010436,gen.XM_{—}010436

FIG. 6281: DNA327092,XM_{—}115874,gen.XM_{—}115874

FIG. 6282: DNA327093,XM_{—}029461,gen.XM_{—}029461

FIG. 6283: PRO83383

FIG. 6284: DNA327094,XM_{—}017930,gen.XM_{—}017930

FIG. 6285: DNA227656,NM_{—}004208,gen.NM_{—}004208

FIG. 6286: PRO38119

FIG. 6287: DNA273487,NM_{—}004794,gen.NM_{—}004794

FIG. 6288: PRO61470

FIG. 6289: DNA327095,XM_{—}088745,gen.XM_{—}088745

FIG. 6290: PRO83385

FIG. 6291: DNA327096,XM_{—}114708,gen.XM_{—}114708

FIG. 6292: PRO83386

FIG. 6293: DNA327097,NM_{—}016267,gen.NM_{—}016267

FIG. 6294: PRO83387

FIG. 6295A-B: DNA327098,XM_{—}042963,gen.XM_{—}042963

FIG. 6296: PRO83388

FIG. 6297: DNA327099,XM_{—}042968,gen.XM_{—}042968

FIG. 6298: PRO83389

FIG. 6299: DNA327100,XM_{—}093219,gen.XM_{—}093219

FIG. 6300: DNA327101,NM_{—}016249,gen.NM_{—}016249

FIG. 6301: PRO83391

FIG. 6302: DNA327102,XM_{—}098995,gen.XM_{—}098995

FIG. 6303: PRO83392

FIG. 6304: DNA327103,XM_{—}041921,gen.XM_{—}041921

FIG. 6305: PRO83393

FIG. 6306: DNA327104,XM_{—}048905,gen.XM_{—}048905

FIG. 6307: PRO83394

FIG. 6308: DNA327105,NM_{—}005364,gen.NM_{—}005364

FIG. 6309: PRO83395

FIG. 6310: DNA327106,XM_{—}010178,gen.XM_{—}010178

FIG. 6311: DNA327107,XM_{—}088592,gen.XM_{—}088592

FIG. 6312: PRO25245

FIG. 6313: DNA327108,XM_{—}018108,gen.XM_{—}018108

FIG. 6314: PRO83397

FIG. 6315: DNA327109,XM_{—}018109,gen.XM_{—}018109

FIG. 6316: DNA327110,NM_{—}005362,gen.NM_{—}005362

FIG. 6317: PRO24021

FIG. 6318: DNA254783,NM_{—}001363,gen.NM_{—}001363

FIG. 6319: PRO49881

FIG. 6320: DNA327111,XM_{—}049337,gen.XM_{—}049337

FIG. 6321: DNA227917,NM_{—}019848,gen.NM_{—}019848

FIG. 6322: PRO38380

FIG. 6323: DNA327112,NM_{—}004699,gen.NM_{—}004699

FIG. 6324: PRO83400

FIG. 6325: DNA327113,XM_{—}048420,gen.XM_{—}048420

FIG. 6326: DNA327114,NM_{—}006013,gen.NM_{—}006013

FIG. 6327: PRO62466

FIG. 6328: DNA327115,XM_{—}048410,gen.XM_{—}048410

FIG. 6329A-C: DNA327116,XM_{—}048404,gen.XM_{—}048404

FIG. 6330A-C: DNA327117,NM_{—}004992,gen.NM_{—}004992

FIG. 6331: PRO83403

FIG. 6332: DNA227013,NM_{—}001569,gen.NM_{—}001569

FIG. 6333: PRO37476

FIG. 6334A-B: DNA225800,NM_{—}000425,gen.NM_{—}000425

FIG. 6335: PRO36263

FIG. 6336A-B: DNA327118,NM_{—}024003,gen.NM_{—}024003

FIG. 6337: PRO83404

FIG. 6338: DNA225655,NM_{—}006280,gen.NM_{—}006280

FIG. 6339: PRO36118

FIG. 6340: DNA276159,NM_{—}004135,gen.NM_{—}004135

FIG. 6341: PRO63299

FIG. 6342A-B: DNA230792,NM_{—}000033,gen.NM_{—}000033

FIG. 6343: PRO38730

FIG. 6344: DNA103558,NM_{—}005745,gen.NM_{—}005745

FIG. 6345: PRO4885

FIG. 6346: DNA327119,XM_{—}042155,gen.XM_{—}042155

FIG. 6347: PRO83405

FIG. 6348: DNA327120,XM_{—}042153,gen.XM_{—}042153

FIG. 6349: DNA327121,XM_{—}117555,gen.XM_{—}117555

FIG. 6350: DNA327122,XM_{—}084311,gen.XM_{—}084311

FIG. 6351: DNA327123,XM_{—}033232,gen.XM_{—}033232

FIG. 6352: DNA327124,XM_{—}117539,gen.XM_{—}117539

FIG. 6353: DNA327125,XM_{—}027952,gen.XM_{—}027952

FIG. 6354: DNA327126,XM_{—}114692,gen.XM_{—}114692

FIG. 6355A-B: DNA327127,XM_{—}165530,gen.XM_{—}165530

DNA Index (to Figure number)

| | |
|---|---|
| DNA0, 1188 | DNA171408, 48 |
| DNA103214, 218 | DNA188229, 5836 |
| DNA103217, 649 | DNA188351, 4782 |
| DNA103239, 5576 | DNA188396, 3480 |
| DNA103253, 188 | DNA188732, 5882 |
| DNA103320, 5272 | DNA188740, 6027 |
| DNA103380, 1677 | DNA188748, 146 |
| DNA103401, 4708 | DNA189315, 167 |
| DNA103421, 2982 | DNA189687, 3297 |
| DNA103436, 457 | DNA189697, 998 |
| DNA103462, 5994 | DNA189703, 4568 |
| DNA103471, 2070 | DNA193882, 585 |
| DNA103474, 3313 | DNA193955, 2193 |
| DNA103486, 5844 | DNA193957, 2947 |
| DNA103505, 1149 | DNA194600, 428 |
| DNA103506, 2990 | DNA194701, 5747 |
| DNA103509, 4110 | DNA194740, 854 |
| DNA103514, 3478 | DNA194805, 4530 |
| DNA103525, 5774 | DNA194807, 5760 |
| DNA103558, 6344 | DNA194827, 977 |
| DNA103580, 5494 | DNA196344, 576 |
| DNA103588, 2274 | DNA196349, 124 |
| DNA103593, 711 | DNA196351, 3600 |
| DNA129504, 4985 | DNA196642, 4877 |
| DNA131588, 2593 | DNA210134, 367 |
| DNA137231, 3667 | DNA210180, 3962 |
| DNA139747, 1368 | DNA218271, 5258 |
| DNA144601, 3051 | DNA218841, 2782 |
| DNA150457, 4936 | DNA219225, 6075 |
| DNA150485, 4305 | DNA219233, 4182 |
| DNA150548, 5703 | DNA225584, 1489 |
| DNA150562, 1153 | DNA225592, 1330 |
| DNA150679, 1732 | DNA225630, 2767 |
| DNA150725, 806 | DNA225631, 2174 |
| DNA150767, 5721 | DNA225632, 3473 |
| DNA150772, 2034 | DNA225649, 4042 |
| DNA150784, 5502 | DNA225655, 6338 |
| DNA150814, 4953 | DNA225671, 2506 |
| DNA150884, 1024 | DNA225721, 6214 |
| DNA150974, 3204 | DNA225752, 3376 |
| DNA150976, 1145 | DNA225800, 6334 |
| DNA150978, 3520 | DNA225809, 356 |
| DNA150997, 3526 | DNA225865, 3976 |
| DNA151010, 2546 | DNA225909, 1828 |
| DNA151017, 1066 | DNA225910, 1128 |
| DNA151148, 44 | DNA225919, 1446 |
| DNA151752, 6020 | DNA225920, 1511 |
| DNA151808, 5476 | DNA225921, 1515 |
| DNA151827, 3466 | DNA225954, 5947 |
| DNA151831, 4141 | DNA226005, 553 |
| DNA151882, 6005 | DNA226011, 5517 |
| DNA151893, 4079 | DNA226014, 3729 |
| DNA151898, 5896 | DNA226028, 3489 |
| DNA226080, 3206 | DNA227491, 2691 |
| DNA226105, 3992 | DNA227504, 594 |
| DNA226125, 409 | DNA227509, 3076 |
| DNA226217, 3004 | DNA227528, 803 |
| DNA226260, 271 | DNA227529, 346 |
| DNA226262, 105 | DNA227545, 698 |
| DNA226324, 4095 | DNA227559, 4161 |
| DNA226337, 2458 | DNA227575, 1508 |
| DNA226345, 2670 | DNA227577, 374 |
| DNA226389, 4820 | DNA227607, 1961 |
| DNA226409, 5921 | DNA227656, 6285 |
| DNA226416, 2262 | DNA227689, 6147 |
| DNA226418, 1791 | DNA227764, 4891 |
| DNA226428, 741 | DNA227795, 792 |
| DNA226496, 2565 | DNA227821, 36 |
| DNA226547, 1108 | DNA227873, 4841 |
| DNA226560, 2393 | DNA227917, 6321 |
| DNA226561, 5956 | DNA227924, 2099 |
| DNA226617, 5935 | DNA227929, 2206 |
| DNA226619, 474 | DNA227943, 6197 |
| DNA226646, 4224 | DNA230792, 6342 |
| DNA226758, 5745 | DNA234442, 4214 |
| DNA226771, 3498 | DNA237931, 6104 |
| DNA226793, 436 | DNA238039, 6181 |
| DNA226853, 3866 | DNA247474, 578 |
| DNA226872, 1689 | DNA247595, 2182 |
| DNA227013, 6332 | DNA251057, 5515 |
| DNA227055, 4939 | DNA252367, 1081 |
| DNA227071, 4889 | DNA253804, 1370 |
| DNA227084, 4742 | DNA254141, 6003 |
| DNA227088, 3220 | DNA254147, 1627 |
| DNA227092, 3593 | DNA254165, 6068 |
| DNA227094, 3628 | DNA254186, 3329 |
| DNA227165, 684 | DNA254198, 4719 |
| DNA227171, 3724 | DNA254204, 994 |
| DNA227172, 2964 | DNA254240, 6045 |
| DNA227173, 1573 | DNA254298, 499 |
| DNA227181, 6272 | DNA254346, 603 |
| DNA227190, 814 | DNA254532, 4487 |
| DNA227191, 3588 | DNA254543, 2740 |
| DNA227204, 1886 | DNA254548, 5627 |
| DNA227206, 4170 | DNA254572, 5885 |
| DNA227213, 157 | DNA254582, 1155 |
| DNA227234, 4626 | DNA254620, 1316 |
| DNA227246, 550 | DNA254624, 3468 |
| DNA227249, 5352 | DNA254771, 2693 |
| DNA227267, 2512 | DNA254777, 3777 |
| DNA227268, 2242 | DNA254781, 4374 |
| DNA227280, 5232 | DNA254783, 6318 |
| DNA227307, 1165 | DNA254785, 6257 |
| DNA227320, 1812 | DNA254791, 4898 |
| DNA227321, 3984 | DNA254994, 5890 |
| DNA227348, 5681 | DNA255046, 5939 |
| DNA227442, 1942 | DNA255078, 3113 |
| DNA227472, 5771 | DNA255340, 4208 |
| DNA227474, 3720 | DNA255370, 4265 |
| DNA255414, 4747 | DNA270721, 3295 |
| DNA255531, 859 | DNA270901, 4879 |
| DNA255696, 3109 | DNA270931, 5504 |
| DNA256070, 6101 | DNA270954, 6079 |
| DNA256072, 3511 | DNA270975, 4843 |
| DNA256503, 199 | DNA270979, 4805 |
| DNA256533, 5513 | DNA270991, 2662 |
| DNA256555, 5146 | DNA271003, 288 |
| DNA256813, 5056 | DNA271010, 5676 |
| DNA256836, 5387 | DNA271040, 1997 |
| DNA256840, 5434 | DNA271060, 751 |
| DNA256844, 4362 | DNA271171, 4507 |
| DNA256886, 4370 | DNA271187, 1093 |
| DNA256905, 545 | DNA271243, 703 |
| DNA257253, 1642 | DNA271324, 3380 |
| DNA257309, 2746 | DNA271344, 3550 |
| DNA257428, 4854 | DNA271418,2104 |
| DNA257511, 1437 | DNA271492, 3727 |
| DNA257531, 5506 | DNA271580, 6187 |
| DNA257549, 5965 | DNA271608, 934 |
| DNA257916, 402 | DNA271626, 1721 |
| DNA257965, 3415 | DNA271722, 2751 |
| DNA269431, 3101 | DNA271841, 5052 |
| DNA269481, 5593 | DNA271843, 3392 |
| DNA269498, 4059 | DNA271847, 2660 |
| DNA269526, 5814 | DNA271931, 1697 |
| DNA269593, 1854 | DNA271986, 519 |
| DNA269630, 5312 | DNA272024, 202 |
| DNA269708, 267 | DNA272050, 2600 |
| DNA269730, 1195 | DNA272062, 5625 |
| DNA269746, 5873 | DNA272090, 2348 |
| DNA269793, 6126 | DNA272127, 881 |
| DNA269803, 3284 | DNA272171, 1866 |
| DNA269809, 1687 | DNA272213, 2734 |
| DNA269816, 1646 | DNA272263, 1967 |
| DNA269830, 5989 | DNA272347, 5426 |
| DNA269858, 1270 | DNA272379, 3555 |
| DNA269894, 5298 | DNA272413, 3390 |
| DNA269910, 1062 | DNA272421, 5201 |
| DNA269930, 1097 | DNA272605, 1335 |
| DNA269952, 3093 | DNA272655, 2714 |
| DNA270015, 3864 | DNA272728, 3215 |
| DNA270134, 3208 | DNA272748, 235 |
| DNA270154, 746 | DNA272889, 4812 |
| DNA270254, 3896 | DNA273014, 4267 |
| DNA270315, 5206 | DNA273060, 194 |
| DNA270401, 1099 | DNA273066, 5568 |
| DNA270458, 3591 | DNA273088, 396 |
| DNA270496, 6239 | DNA273254, 6230 |
| DNA270613, 1892 | DNA273320, 5785 |
| DNA270615, 1386 | DNA273346, 5615 |
| DNA270621, 5234 | DNA273474, 5421 |
| DNA270675, 1850 | DNA273487, 6287 |
| DNA270677, 3823 | DNA273517, 5738 |
| DNA270697, 6011 | DNA273521, 3066 |
| DNA270711, 2371 | DNA273600, 5448 |
| DNA273694, 5023 | DNA287290, 5685 |
| DNA273712, 42 | DNA287291, 4919 |
| DNA273759, 2899 | DNA287319, 1969 |
| DNA273800, 689 | DNA287331, 4242 |
| DNA273839, 4360 | DNA287355, 4520 |
| DNA273865, 2246 | DNA287417, 3218 |
| DNA273919, 1182 | DNA287425, 4900 |
| DNA273992, 6190 | DNA287427, 4778 |
| DNA274002, 4476 | DNA287636, 5154 |
| DNA274034, 5277 | DNA287642, 2951 |
| DNA274058, 3912 | DNA288247, 2703 |
| DNA274101, 5115 | DNA288259, 1598 |
| DNA274129, 5892 | DNA289522, 4446 |
| DNA274139, 5441 | DNA289530, 2761 |
| DNA274178, 2491 | DNA290231, 1638 |
| DNA274180, 4516 | DNA290234, 540 |
| DNA274206, 1830 | DNA290259, 6034 |
| DNA274289, 5523 | DNA290260, 5550 |
| DNA274326, 2176 | DNA290264, 2007 |
| DNA274361, 3763 | DNA290284, 350 |
| DNA274487, 180 | DNA290292, 4728 |
| DNA274690, 5039 | DNA290294, 3620 |
| DNA274745, 192 | DNA290319,2680 |
| DNA274755, 4975 | DNA290585, 1459 |
| DNA274759, 340 | DNA290785, 2032 |
| DNA274761, 5199 | DNA294794, 438 |
| DNA274823, 5542 | DNA297288, 5638 |
| DNA274829, 6149 | DNA297388, 4699 |
| DNA275049, 662 | DNA297398, 3434 |
| DNA275066, 744 | DNA299899, 930 |
| DNA275139, 292 | DNA302016, 3827 |
| DNA275144, 4300 | DNA302020, 1718 |
| DNA275181, 4320 | DNA304459, 2986 |
| DNA275195, 651 | DNA304460, 1908 |
| DNA275240, 864 | DNA304488, 2996 |
| DNA275322, 2723 | DNA304658, 5562 |
| DNA275334, 2232 | DNA304661, 1946 |
| DNA275408, 4564 | DNA304662, 5640 |
| DNA275630, 1904 | DNA304666, 369 |
| DNA276159, 6340 | DNA304668, 1963 |
| DNA281436, 3900 | DNA304669, 3887 |
| DNA287167, 794 | DNA304670, 5830 |
| DNA287173, 31 | DNA304680, 1874 |
| DNA287189, 2265 | DNA304685, 2435 |
| DNA287216, 2701 | DNA304686, 220 |
| DNA287227, 1952 | DNA304694, 3717 |
| DNA287234, 5014 | DNA304699, 1986 |
| DNA287237, 3008 | DNA304704, 4575 |
| DNA287240, 5328 | DNA304707, 2254 |
| DNA287243, 5279 | DNA304710, 2308 |
| DNA287246, 1900 | DNA304715, 4714 |
| DNA287254, 3236 | DNA304716, 1912 |
| DNA287261, 5668 | DNA304719, 6038 |
| DNA287270, 5654 | DNA304720, 371 |
| DNA287271, 2763 | DNA304783, 3631 |
| DNA287282, 1582 | DNA304801, 2342 |
| DNA304805, 905 | DNA323771, 98 |
| DNA304835, 5973 | DNA323772, 99 |
| DNA323717, 1 | DNA323773, 101 |
| DNA323718, 2 | DNA323774, 102 |
| DNA323719, 3 | DNA323775, 103 |
| DNA323720, 4 | DNA323776, 107 |
| DNA323721, 6 | DNA323777, 109 |
| DNA323722, 8 | DNA323778, 110 |
| DNA323723, 10 | DNA323779, 112 |
| DNA323724, 12 | DNA323780, 113 |
| DNA323725, 14 | DNA323781, 114 |
| DNA323726, 15 | DNA323782, 116 |
| DNA323727, 17 | DNA323783, 118 |
| DNA323728, 19 | DNA323784, 120 |
| DNA323729, 20 | DNA323785, 122 |
| DNA323730, 22 | DNA323788, 126 |
| DNA323731, 24 | DNA323789, 127 |
| DNA323732, 26 | DNA323790, 129 |
| DNA323733, 28 | DNA323791, 130 |
| DNA323734, 29 | DNA323792, 131 |
| DNA323735, 33 | DNA323793, 133 |
| DNA323736, 34 | DNA323794, 134 |
| DNA323737, 38 | DNA323795, 135 |
| DNA323738, 40 | DNA323796, 136 |
| DNA323739, 41 | DNA323797, 137 |
| DNA323740, 46 | DNA323798, 139 |
| DNA323741, 50 | DNA323799, 140 |
| DNA323742, 52 | DNA323800, 141 |
| DNA323743, 54 | DNA323801, 142 |
| DNA323744, 55 | DNA323802, 144 |
| DNA323745, 57 | DNA323803, 145 |
| DNA323746, 58 | DNA323804, 148 |
| DNA323747, 59 | DNA323805, 150 |
| DNA323748, 60 | DNA323806, 152 |
| DNA323749, 62 | DNA323807, 154 |
| DNA323750, 64 | DNA323 808, 155 |
| DNA323751, 66 | DNA323809, 159 |
| DNA323752, 67 | DNA323810, 161 |
| DNA323753, 68 | DNA323811, 163 |
| DNA323754, 69 | DNA323812, 165 |
| DNA323755, 71 | DNA323813, 169 |
| DNA323756, 73 | DNA323814, 171 |
| DRA323757, 75 | DNA323815, 175 |
| DNA323758, 76 | DNA323816, 176 |
| DNA323759, 77 | DNA323817, 178 |
| DNA323760, 78 | DNA323818, 182 |
| DNA323761, 79 | DNA323819, 183 |
| DNA323762, 81 | DNA323820, 185 |
| DNA323763, 83 | DNA323821, 187 |
| DNA323764, 85 | DNA323822, 190 |
| DNA323765, 87 | DNA323823, 196 |
| DNA323766, 89 | DNA323824, 198 |
| DNA323767, 91 | DNA323825, 201 |
| DNA323768, 93 | DNA323826, 204 |
| DNA323769, 95 | DNA323827, 206 |
| DNA323770, 97 | DNA323828, 208 |
| DNA323829, 210 | DNA323885, 317 |
| DNA323830, 212 | DNA323886, 318 |
| DNA323831, 213 | DNA323887, 319 |
| DNA323832, 214 | DNA323888, 321 |
| DNA323833, 216 | DNA323889, 323 |
| DNA323834, 222 | DNA323890, 324 |
| DNA323835, 224 | DNA323891, 326 |
| DNA323836, 226 | DNA323892, 327 |
| DNA323837, 228 | DNA323893, 328 |
| DNA323838, 229 | DNA323894, 330 |
| DNA323839, 231 | DNA323895, 331 |
| DNA323840, 233 | DNA323896, 332 |
| DNA323841, 237 | DNA323897, 334 |
| DNA323842, 239 | DNA323898, 336 |
| DNA323843, 241 | DNA323899, 338 |
| DNA323844, 243 | DNA323900, 342 |
| DNA323845, 244 | DNA323901, 344 |
| DNA323846, 245 | DNA323902, 348 |
| DNA323847, 247 | DNA323903, 352 |
| DNA323848, 249 | DNA323904, 353 |
| DNA323849, 250 | DNA323905, 354 |
| DNA323850, 251 | DNA323906, 358 |
| DNA323851, 253 | DNA323907, 360 |
| DNA323852, 254 | DNA323908, 361 |
| DNA323853, 256 | DNA323909, 363 |
| DNA323854, 257 | DNA323910, 364 |
| DNA323855, 259 | DNA323911, 366 |
| DNA323856, 260 | DNA323912, 373 |
| DNA323857, 262 | DNA323913, 376 |
| DNA323858, 264 | DNA323914, 377 |
| DNA323859, 265 | DNA323915, 379 |
| DNA323860, 269 | DNA323916, 381 |
| DNA323861, 273 | DNA323917, 383 |
| DNA323862, 275 | DNA323918, 384 |
| DNA323863, 276 | DNA323919, 386 |
| DNA323864, 277 | DNA323920, 388 |
| DNA323865, 279 | DNA323921, 389 |
| DNA323866, 280 | DNA323922, 391 |
| DNA323867, 281 | DNA323923, 392 |
| DNA323868, 282 | DNA323924, 394 |
| DNA323869, 284 | DNA323925, 398 |
| DNA323870, 286 | DNA323926, 400 |
| DNA323871, 290 | DNA323927, 404 |
| DNA323872, 294 | DNA323928, 406 |
| DNA323873, 295 | DNA323929, 408 |
| DNA323874, 296 | DNA323930, 411 |
| DNA323875, 298 | DNA323931, 412 |
| DNA323876, 300 | DNA323932, 414 |
| DNA323877, 302 | DNA323933, 416 |
| DNA323 878, 304 | DNA323934, 418 |
| DNA323879, 306 | DNA323935, 420 |
| DNA323880, 308 | DNA323936, 422 |
| DNA323881, 310 | DNA323937, 424 |
| DNA323882, 312 | DNA323938, 426 |
| DNA323883, 314 | DNA323939, 430 |
| DNA323884, 315 | DNA323940, 432 |
| DNA323941, 433 | DNA323997, 537 |
| DNA323942, 434 | DNA323998, 538 |
| DNA323943, 440 | DNA323999, 542 |
| DNA323944, 442 | DNA324000, 543 |
| DNA323945, 444 | DNA324001, 544 |
| DNA323946, 446 | DNA324002, 547 |
| DNA323947, 448 | DNA324003, 548 |
| DNA323948, 450 | DNA324004, 552 |
| DNA323949, 451 | DNA324005, 555 |
| DNA323950, 452 | DNA324006, 557 |
| DNA323951, 454 | DNA324007, 560 |
| DNA323952, 455 | DNA324008, 561 |
| DNA323953, 459 | DNA324009, 562 |
| DNA323954, 461 | DNA324010, 564 |
| DNA323955, 463 | DNA324011, 566 |
| DNA323956, 465 | DNA324012, 567 |
| DNA323957, 466 | DNA324013, 568 |
| DNA323958, 468 | DNA324014, 569 |
| DNA323959, 470 | DNA324015, 571 |
| DNA323960, 472 | DNA324016, 573 |
| DNA323961, 473 | DNA324017, 575 |
| DNA323962, 476 | DNA324018, 580 |
| DNA323963, 477 | DNA324019, 581 |
| DNA323964, 479 | DNA324020, 582 |
| DNA323965, 481 | DNA324021, 583 |
| DNA323966, 483 | DNA324022, 587 |
| DNA323967, 484 | DNA324023, 589 |
| DNA323968, 485 | DNA324024, 591 |
| DNA323969, 486 | DNA324025, 592 |
| DNA323970, 488 | DNA324026, 593 |
| DNA323971, 490 | DNA324027, 596 |
| DNA323972, 492 | DNA324028, 598 |
| DNA323973, 493 | DNA324029, 599 |
| DNA323974, 494 | DNA324030, 600 |
| DNA323975, 495 | DNA324031, 601 |
| DNA323976, 497 | DNA324032, 602 |
| DNA323977, 501 | DNA324033, 605 |
| DNA323978, 503 | DNA324034, 606 |
| DNA323979, 505 | DNA324035, 608 |
| DNA323980, 507 | DNA324036, 610 |
| DNA323981, 509 | DNA324037, 612 |
| DNA323982, 511 | DNA324038, 614 |
| DNA323983, 513 | DNA324039, 616 |
| DNA323984, 515 | DNA324040, 618 |
| DNA323985, 517 | DNA324041, 619 |
| DNA323986, 521 | DNA324042, 620 |
| DNA323987, 522 | DNA324043, 622 |
| DNA323988, 523 | DNA324044, 626 |
| DNA323989, 524 | DNA324045, 628 |
| DNA323990, 525 | DNA324046, 630 |
| DNA323991, 527 | DNA324047, 632 |
| DNA323992, 529 | DNA324048, 634 |
| DNA323993, 531 | DNA324049, 636 |
| DNA323994, 532 | DNA324050, 638 |
| DNA323995, 534 | DNA324051, 639 |
| DNA323996, 535 | DNA324052, 641 |
| DNA324053, 642 | DNA324109, 763 |
| DNA324054, 643 | DNA324110, 764 |
| DNA324055, 645 | DNA324111, 766 |
| DNA324056, 647 | DNA324112, 768 |
| DNA324057, 653 | DNA324113, 770 |
| DNA324058, 655 | DNA324114, 771 |
| DNA324059, 657 | DNA324115, 772 |
| DNA324060, 659 | DNA324116, 773 |
| DNA324061, 661 | DNA324117, 775 |
| DNA324062, 664 | DNA324118, 776 |
| DNA324063, 665 | DNA324119, 777 |
| DNA324064, 667 | DNA324120, 779 |
| DNA324065, 669 | DNA324121, 780 |
| DNA324066, 670 | DNA324122, 782 |
| DNA324067, 672 | DNA324123, 783 |
| DNA324068, 674 | DNA324124, 784 |
| DNA324069, 676 | DNA324125, 785 |
| DNA324070, 678 | DNA324126, 787 |
| DNA324071, 680 | DNA324127, 788 |
| DNA324072, 681 | DNA324128, 789 |
| DNA324073, 683 | DNA324129, 791 |
| DNA324074, 686 | DNA324130, 796 |
| DNA324075, 690 | DNA324131, 798 |
| DNA324076, 692 | DNA324132, 800 |
| DNA324077, 694 | DNA324133, 801 |
| DNA324078, 696 | DNA324134, 805 |
| DNA324079, 700 | DNA324135, 808 |
| DNA324080, 701 | DNA324136, 810 |
| DNA324081, 705 | DNA324137, 812 |
| DNA324082, 707 | DNA324138, 816 |
| DNA324083, 709 | DNA324139, 817 |
| DNA324084, 713 | DNA324140, 818 |
| DNA324085, 715 | DNA324141, 820 |
| DNA324086, 716 | DNA324142, 822 |
| DNA324087, 717 | DNA324143, 823 |
| DNA324088, 719 | DNA324144, 824 |
| DNA324089, 721 | DNA324145, 825 |
| DNA324090, 723 | DNA324146, 827 |
| DNA324091, 725 | DNA324147, 829 |
| DNA324092, 726 | DNA324148, 831 |
| DNA324093, 727 | DNA324149, 832 |
| DNA324094, 729 | DNA324150, 834 |
| DNA324095, 731 | DNA324151, 836 |
| DNA324096, 733 | DNA324152, 838 |
| DNA324097, 734 | DNA324153, 839 |
| DNA324098, 736 | DNA324154, 841 |
| DNA324099, 738 | DNA324155, 842 |
| DNA324100, 740 | DNA324156, 843 |
| DNA324101, 743 | DNA324157, 845 |
| DNA324102, 748 | DNA324158, 847 |
| DNA324103, 749 | DNA324159, 849 |
| DNA324104, 753 | DNA324160, 850 |
| DNA324105, 755 | DNA324161, 851 |
| DNA324106, 757 | DNA324162, 853 |
| DNA324107, 759 | DNA324163, 856 |
| DNA324108, 761 | DNA324164, 857 |
| DNA324165, 858 | DNA324221, 962 |
| DNA324166, 861 | DNA324222, 964 |
| DNA324167, 862 | DNA324223, 965 |
| DNA324168, 866 | DNA324224, 966 |
| DNA324169, 867 | DNA324225, 968 |
| DNA324170, 869 | DNA324226, 970 |
| DNA324171, 871 | DNA324227, 971 |
| DNA324172, 873 | DNA324228, 973 |
| DNA324173, 875 | DNA324229, 975 |
| DNA324174, 877 | DNA324230, 979 |
| DNA324175, 878 | DNA324231, 980 |
| DNA324176, 880 | DNA324232, 982 |
| DNA324177, 883 | DNA324233, 984 |
| DNA324178, 885 | DNA324234, 985 |
| DNA324179, 887 | DNA324235, 986 |
| DNA324180, 889 | DNA324236, 988 |
| DNA324181, 891 | DNA324237, 990 |
| DNA324182, 893 | DNA324238, 992 |
| DNA324183, 894 | DNA324239, 993 |
| DNA324184, 896 | DNA324240, 996 |
| DNA324185, 900 | DNA324241, 1000 |
| DNA324186, 901 | DNA324242, 1002 |
| DNA324187, 903 | DNA324243, 1004 |
| DNA324188, 907 | DNA324244, 1006 |
| DNA324189, 909 | DNA324245, 1007 |
| DNA324190, 910 | DNA324246, 1009 |
| DNA324191, 911 | DNA324247, 1011 |
| DNA324192, 912 | DNA324248, 1012 |
| DNA324193, 914 | DNA324249, 1014 |
| DNA324194, 916 | DNA324250, 1016 |
| DNA324195, 918 | DNA324251, 1018 |
| DNA324196, 920 | DNA324252, 1020 |
| DNA324197, 921 | DNA324253, 1022 |
| DNA324198, 923 | DNA324254, 1026 |
| DNA324199, 925 | DNA324255, 1028 |
| DNA324200, 926 | DNA324256, 1029 |
| DNA324201, 927 | DNA324257, 1030 |
| DNA324202, 928 | DNA324258, 1032 |
| DNA324203, 929 | DNA324259, 1034 |
| DNA324204, 932 | DNA324260, 1036 |
| DNA324205, 933 | DNA324261, 1037 |
| DNA324206, 936 | DNA324262, 1039 |
| DNA324207, 938 | DNA324263, 1040 |
| DNA324208, 940 | DNA324264, 1041 |
| DNA324209, 941 | DNA324265, 1042 |
| DNA324210, 942 | DNA324266, 1043 |
| DNA324211, 944 | DNA324267, 1045 |
| DNA324212, 946 | DNA324268, 1047 |
| DNA324213, 948 | DNA324269, 1049 |
| DNA324214, 950 | DNA324270, 1051 |
| DNA324215, 952 | DNA324271, 1053 |
| DNA324216, 954 | DNA324272, 1055 |
| DNA324217, 955 | DNA324273, 1057 |
| DNA324218, 957 | DNA324274, 1059 |
| DNA324219, 958 | DNA324275, 1060 |
| DNA324220, 960 | DNA324276, 1064 |
| DNA324277, 1068 | DNA324333, 1186 |
| DNA324278, 1070 | DNA324334, 1187 |
| DNA324279, 1072 | DNA324335, 1190 |
| DNA324280, 1074 | DNA324336, 1192 |
| DNA324281, 1075 | DNA324337, 1193 |
| DNA324282, 1076 | DNA324338, 1197 |
| DNA324283, 1078 | DNA324339, 1198 |
| DNA324284, 1079 | DNA324340, 1199 |
| DNA324285, 1083 | DNA324341, 1201 |
| DNA324286, 1085 | DNA324342, 1202 |
| DNA324287, 1086 | DNA324343, 1203 |
| DNA324288, 1088 | DNA324344, 1204 |
| DNA324289, 1091 | DNA324345, 1205 |
| DNA324290, 1095 | DNA324346, 1206 |
| DNA324291, 1101 | DNA324347, 1208 |
| DNA324292, 1103 | DNA324348, 1209 |
| DNA324293, 1105 | DNA324349, 1211 |
| DNA324294, 1106 | DNA324350, 1213 |
| DNA324295, 1110 | DNA324351, 1214 |
| DNA324296, 1112 | DNA324352, 1216 |
| DNA324297, 1113 | DNA324353, 1218 |
| DNA324298, 1115 | DNA324354, 1220 |
| DNA324299, 1117 | DNA324355, 1221 |
| DNA324300, 1119 | DNA324356, 1225 |
| DNA324301, 1120 | DNA324357, 1227 |
| DNA324302, 1121 | DNA324358, 1229 |
| DNA324303, 1122 | DNA324359, 1231 |
| DNA324304, 1123 | DNA324360, 1232 |
| DNA324305, 1125 | DNA324361, 1234 |
| DNA324306, 1127 | DNA324362, 1235 |
| DNA324307, 1130 | DNA324363, 1237 |
| DNA324308, 1131 | DNA324364, 1238 |
| DNA324309, 1132 | DNA324365, 1240 |
| DNA324310, 1134 | DNA324366, 1242 |
| DNA324311, 1136 | DNA324367, 1243 |
| DNA324312, 1137 | DNA324368, 1244 |
| DNA324313, 1139 | DNA324369, 1245 |
| DNA324314, 1140 | DNA324370, 1246 |
| DNA324315, 1141 | DNA324371, 1248 |
| DNA324316, 1143 | DNA324372, 1250 |
| DNA324317, 1147 | DNA324373, 1252 |
| DNA324318, 1151 | DNA324374, 1254 |
| DNA324319, 1157 | DNA324375, 1255 |
| DNA324320, 1159 | DNA324376, 1256 |
| DNA324321, 1161 | DNA324377, 1258 |
| DNA324322, 1162 | DNA324378, 1260 |
| DNA324323, 1163 | DNA324379, 1262 |
| DNA324324, 1167 | DNA324380, 1263 |
| DNA324325, 1169 | DNA324381, 1264 |
| DNA324326, 1170 | DNA324382, 1265 |
| DNA324327, 1172 | DNA324383, 1266 |
| DNA324328, 1174 | DNA324384, 1267 |
| DNA324329, 1176 | DNA324385, 1268 |
| DNA324330, 1178 | DNA324386, 1272 |
| DNA324331, 1180 | DNA324387, 1274 |
| DNA324332, 1184 | DNA324388, 1275 |
| DNA324389, 1276 | DNA324445, 1376 |
| DNA324390, 1278 | DNA324446, 1378 |
| DNA324391, 1280 | DNA324447, 1380 |
| DNA324392, 1282 | DNA324448, 1382 |
| DNA324393, 1284 | DNA324449, 1384 |
| DNA324394, 1286 | DNA324450, 1388 |
| DNA324395, 1288 | DNA324451, 1390 |
| DNA324396, 1289 | DNA324452, 1392 |
| DNA324397, 1290 | DNA324453, 1394 |
| DNA324398, 1291 | DNA324454, 1396 |
| DNA324399, 1292 | DNA324455, 1398 |
| DNA324400, 1294 | DNA324456, 1400 |
| DNA324401, 1295 | DNA324457, 1402 |
| DNA324402, 1296 | DNA324458, 1404 |
| DNA324403, 1297 | DNA324459, 1406 |
| DNA324404, 1299 | DNA324460, 1408 |
| DNA324405, 1300 | DNA324461, 1410 |
| DNA324406, 1302 | DNA324462, 1412 |
| DNA324407, 1304 | DNA324463, 1413 |
| DNA324408, 1306 | DNA324464, 1414 |
| DNA324409, 1308 | DNA324465, 1416 |
| DNA324410, 1310 | DNA324466, 1417 |
| DNA324411, 1312 | DNA324467, 1418 |
| DNA324412, 1313 | DNA324468, 1419 |
| DNA324413, 1314 | DNA324469, 1421 |
| DNA324414, 1315 | DNA324470, 1423 |
| DNA324415, 1318 | DNA324471, 1424 |
| DNA324416, 1320 | DNA324472, 1425 |
| DNA324417, 1322 | DNA324473, 1427 |
| DNA324418, 1323 | DNA324474, 1429 |
| DNA324419, 1325 | DNA324475, 1430 |
| DNA324420, 1329 | DNA324476, 1432 |
| DNA324421, 1332 | DNA324478, 1433 |
| DNA324422, 1333 | DNA324479, 1434 |
| DNA324423, 1337 | DNA324480, 1435 |
| DNA324424, 1338 | DNA324481, 1439 |
| DNA324425, 1340 | DNA324482, 1440 |
| DNA324426, 1341 | DNA324483, 1441 |
| DNA324427, 1343 | DNA324484, 1442 |
| DNA324428, 1344 | DNA324485, 1443 |
| DNA324429, 1345 | DNA324486, 1445 |
| DNA324430, 1347 | DNA324487, 1448 |
| DNA324431, 1348 | DNA324488, 1449 |
| DNA324432, 1350 | DNA324489, 1451 |
| DNA324433, 1354 | DNA324490, 1452 |
| DNA324434, 1356 | DNA324491, 1453 |
| DNA324435, 1358 | DNA324492, 1455 |
| DNA324436, 1359 | DNA324493, 1456 |
| DNA324437, 1360 | DNA324494, 1457 |
| DNA324438, 1361 | DNA324495, 1461 |
| DNA324439, 1363 | DNA324496, 1463 |
| DNA324440, 1364 | DNA324497, 1464 |
| DNA324441, 1365 | DNA324498, 1465 |
| DNA324442, 1366 | DNA324499, 1466 |
| DNA324443, 1372 | DNA324500, 1468 |
| DNA324444, 1374 | DNA324501, 1469 |
| DNA324502, 1470 | DNA324559, 1556 |
| DNA324503, 1471 | DNA324560, 1557 |
| DNA324504, 1472 | DNA324561, 1559 |
| DNA324505, 1473 | DNA324562, 1561 |
| DNA324506, 1474 | DNA324563, 1562 |
| DNA324507, 1476 | DNA324564, 1564 |
| DNA324508, 1477 | DNA324565, 1565 |
| DNA324509, 1478 | DNA324566, 1567 |
| DNA324510, 1480 | DNA324567, 1568 |
| DNA324511, 1482 | DNA324568, 1570 |
| DNA324512, 1483 | DNA324569, 1572 |
| DNA324513, 1484 | DNA324570, 1575 |
| DNA324514, 1485 | DNA324571, 1577 |
| DNA324515, 1487 | DNA324572, 1579 |
| DNA324516, 1491 | DNA324573, 1581 |
| DNA324517, 1493 | DNA324574, 1584 |
| DNA324518, 1494 | DNA324575, 1586 |
| DNA324519, 1496 | DNA324576, 1587 |
| DNA324520, 1497 | DNA324577, 1588 |
| DNA324521, 1499 | DNA324578, 1590 |
| DNA324522, 1500 | DNA324579, 1591 |
| DNA324523, 1502 | DNA324580, 1592 |
| DNA324524, 1504 | DNA324581, 1593 |
| DNA324525, 1506 | DNA324582, 1595 |
| DNA324526, 1510 | DNA324583, 1596 |
| DNA324527, 1513 | DNA324584, 1597 |
| DNA324528, 1517 | DNA324585, 1600 |
| DNA324529, 1519 | DNA324586, 1602 |
| DNA324530, 1520 | DNA324587, 1604 |
| DNA324531, 1522 | DNA324588, 1606 |
| DNA324532, 1524 | DNA324589, 1608 |
| DNA324533, 1525 | DNA324590, 1609 |
| DNA324534, 1526 | DNA324591, 1610 |
| DNA324535, 1528 | DNA324592, 1611 |
| DNA324536, 1530 | DNA324593, 1612 |
| DNA324537, 1531 | DNA324594, 1614 |
| DNA324538, 1532 | DNA324595, 1615 |
| DNA324539, 1533 | DNA324596, 1617 |
| DNA324540, 1534 | DNA324597, 1619 |
| DNA324541, 1535 | DNA324598, 1621 |
| DNA324542, 1537 | DNA324599, 1622 |
| DNA324543, 1539 | DNA324600, 1623 |
| DNA324544, 1540 | DNA324601, 1624 |
| DNA324545, 1541 | DNA324602, 1626 |
| DNA324546, 1543 | DNA324603, 1629 |
| DNA324547, 1544 | DNA324604, 1631 |
| DNA324548, 1545 | DNA324605, 1632 |
| DNA324549, 1547 | DNA324606, 1634 |
| DNA324550, 1548 | DNA324607, 1636 |
| DNA324551, 1549 | DNA324608, 1640 |
| DNA324552, 1550 | DNA324609, 1641 |
| DNA324554, 1551 | DNA324610, 1644 |
| DNA324555, 1552 | DNA324611, 1648 |
| DNA324556, 1553 | DNA324612, 1650 |
| DNA324557, 1554 | DNA324613, 1652 |
| DNA324558, 1555 | DNA324614, 1654 |
| DNA324615, 1655 | DNA324671, 1768 |
| DNA324616, 1656 | DNA324672, 1770 |
| DNA324617, 1658 | DNA324673, 1772 |
| DNA324618, 1660 | DNA324674, 1774 |
| DNA324619, 1662 | DNA324675, 1776 |
| DNA324620, 1663 | DNA324676, 1778 |
| DNA324621, 1664 | DNA324677, 1779 |
| DNA324622, 1666 | DNA324678, 1781 |
| DNA324623, 1668 | DNA324679, 1783 |
| DNA324624, 1669 | DNA324680, 1785 |
| DNA324625, 1670 | DNA324681, 1787 |
| DNA324626, 1673 | DNA324682, 1789 |
| DNA324627, 1675 | DNA324683, 1793 |
| DNA324628, 1679 | DNA324684, 1795 |
| DNA324629, 1681 | DNA324685, 1797 |
| DNA324630, 1683 | DNA324686, 1798 |
| DNA324631, 1685 | DNA324687, 1799 |
| DNA324632, 1691 | DNA324688, 1800 |
| DNA324633, 1693 | DNA324689, 1802 |
| DNA324634, 1695 | DNA324690, 1803 |
| DNA324635, 1699 | DNA324691, 1805 |
| DNA324636, 1700 | DNA324692, 1807 |
| DNA324637, 1701 | DNA324693, 1808 |
| DNA324638, 1702 | DNA324694, 1810 |
| DNA324639, 1704 | DNA324695, 1811 |
| DNA324640, 1706 | DNA324696, 1814 |
| DNA324641, 1708 | DNA324697, 1816 |
| DNA324642, 1710 | DNA324698, 1817 |
| DNA324643, 1711 | DNA324699, 1818 |
| DNA324644, 1712 | DNA324700, 1819 |
| DNA324645, 1713 | DNA324701, 1820 |
| DNA324646, 1714 | DNA324702, 1821 |
| DNA324647, 1716 | DNA324703, 1823 |
| DNA324648, 1720 | DNA324704, 1824 |
| DNA324649, 1723 | DNA324705, 1826 |
| DNA324650, 1724 | DNA324706, 1832 |
| DNA324651, 1726 | DNA324707, 1834 |
| DNA324652, 1728 | DNA324708, 1836 |
| DNA324653, 1730 | DNA324709, 1838 |
| DNA324654, 1734 | DNA324710, 1840 |
| DNA324655, 1736 | DNA324711, 1841 |
| DNA324656, 1738 | DNA324712, 1842 |
| DNA324657, 1740 | DNA324713, 1843 |
| DNA324658, 1742 | DNA324714, 1845 |
| DNA324659, 1744 | DNA324715, 1846 |
| DNA324660, 1746 | DNA324716, 1848 |
| DNA324661, 1748 | DNA324717, 1852 |
| DNA324662, 1750 | DNA324718, 1856 |
| DNA324663, 1752 | DNA324719, 1857 |
| DNA324664, 1754 | DNA324720, 1858 |
| DNA324665, 1756 | DNA324721, 1859 |
| DNA324666, 1758 | DNA324722, 1860 |
| DNA324667, 1760 | DNA324723, 1861 |
| DNA324668, 1762 | DNA324724, 1862 |
| DNA324669, 1764 | DNA324725, 1863 |
| DNA324670, 1766 | DNA324726, 1865 |
| DNA324727, 1868 | DNA324784, 1988 |
| DNA324728, 1870 | DNA324785, 1990 |
| DNA324729, 1872 | DNA324786, 1992 |
| DNA324730, 1876 | DNA324787, 1994 |
| DNA324731, 1877 | DNA324788, 1995 |
| DNA324732, 1878 | DNA324789, 1999 |
| DNA324733, 1879 | DNA324790, 2000 |
| DNA324734, 1880 | DNA324791, 2002 |
| DNA324735, 1882 | DNA324792, 2004 |
| DNA324736, 1883 | DNA324793, 2006 |
| DNA324737, 1884 | DNA324794, 2009 |
| DNA324738, 1888 | DNA324795, 2011 |
| DNA324739, 1890 | DNA324796, 2013 |
| DNA324740, 1894 | DNA324797, 2015 |
| DNA324741, 1896 | DNA324798, 2016 |
| DNA324742, 1898 | DNA324799, 2017 |
| DNA324743, 1902 | DNA324800, 2019 |
| DNA324744, 1906 | DNA324801, 2021 |
| DNA324745, 1910 | DNA324802, 2023 |
| DNA324746, 1914 | DNA324803, 2025 |
| DNA324747, 1916 | DNA324804, 2027 |
| DNA324748, 1918 | DNA324805, 2029 |
| DNA324749, 1920 | DNA324806, 2031 |
| DNA324750, 1921 | DNA324807, 2036 |
| DNA324751, 1922 | DNA324808, 2037 |
| DNA324752, 1924 | DNA324809, 2039 |
| DNA324753, 1926 | DNA324810, 2041 |
| DNA324754, 1928 | DNA324811, 2042 |
| DNA324755, 1929 | DNA324812, 2044 |
| DNA324756, 1931 | DNA324813, 2045 |
| DNA324757, 1932 | DNA324814, 2047 |
| DNA324758, 1934 | DNA324815, 2049 |
| DNA324759, 1936 | DNA324816, 2050 |
| DNA324760, 1937 | DNA324817, 2052 |
| DNA324761, 1938 | DNA324818, 2054 |
| DNA324763, 1939 | DNA324819, 2056 |
| DNA324764, 1940 | DNA324820, 2057 |
| DNA324765, 1941 | DNA324821, 2058 |
| DNA324766, 1944 | DNA324822, 2059 |
| DNA324767, 1948 | DNA324823, 2060 |
| DNA324768, 1949 | DNA324824, 2062 |
| DNA324769, 1951 | DNA324825, 2064 |
| DNA324770, 1954 | DNA324826, 2065 |
| DNA324771, 1955 | DNA324827, 2066 |
| DNA324772, 1956 | DNA324828, 2068 |
| DNA324773, 1957 | DNA324829, 2069 |
| DNA324774, 1959 | DNA324830, 2072 |
| DNA324775, 1965 | DNA324831, 2074 |
| DNA324776, 1971 | DNA324832, 2075 |
| DNA324777, 1973 | DNA324833, 2077 |
| DNA324778, 1975 | DNA324834, 2079 |
| DNA324779, 1977 | DNA324835, 2080 |
| DNA324780, 1979 | DNA324836, 2081 |
| DNA324781, 1981 | DNA324837, 2083 |
| DNA324782, 1983 | DNA324838, 2085 |
| DNA324783, 1984 | DNA324839, 2087 |
| DNA324840, 2089 | DNA324897, 2184 |
| DNA324841, 2090 | DNA324898, 2186 |
| DNA324842, 2091 | DNA324899, 2188 |
| DNA324843, 2092 | DNA324900, 2190 |
| DNA324844, 2094 | DNA324901, 2191 |
| DNA324845, 2096 | DNA324902, 2195 |
| DNA324846, 2098 | DNA324903, 2197 |
| DNA324847, 2101 | DNA324904, 2198 |
| DNA324848, 2103 | DNA324905, 2200 |
| DNA324849, 2106 | DNA324906, 2202 |
| DNA324850, 2107 | DNA324907, 2203 |
| DNA324851, 2108 | DNA324908, 2204 |
| DNA324852, 2110 | DNA324909, 2205 |
| DNA324853, 2111 | DNA324910, 2208 |
| DNA324854, 2113 | DNA324911, 2210 |
| DNA324855, 2114 | DNA324912, 2212 |
| DNA324856, 2116 | DNA324913, 2214 |
| DNA324857, 2118 | DNA324914, 2216 |
| DNA324858, 2119 | DNA324915, 2218 |
| DNA324859, 2121 | DNA324916, 2219 |
| DNA324860, 2122 | DNA324917, 2220 |
| DNA324861, 2123 | DNA324918, 2222 |
| DNA324862, 2124 | DNA324919, 2224 |
| DNA324863, 2126 | DNA324920, 2225 |
| DNA324864, 2128 | DNA324921, 2226 |
| DNA324865, 2130 | DNA324922, 2228 |
| DNA324866, 2131 | DNA324923, 2230 |
| DNA324867, 2132 | DNA324924, 2234 |
| DNA324868, 2134 | DNA324925, 2236 |
| DNA324870, 2135 | DNA324926, 2238 |
| DNA324871, 2137 | DNA324927, 2240 |
| DNA324872, 2139 | DNA324928, 2244 |
| DNA324873, 2140 | DNA324929, 2245 |
| DNA324874, 2141 | DNA324930, 2248 |
| DNA324875, 2142 | DNA324931, 2249 |
| DNA324876, 2144 | DNA324932, 2251 |
| DNA324877, 2145 | DNA324933, 2253 |
| DNA324878, 2146 | DNA324934, 2256 |
| DNA324879, 2147 | DNA324935, 2258 |
| DNA324880, 2148 | DNA324936, 2259 |
| DNA324881, 2150 | DNA324937, 2260 |
| DNA324882, 2152 | DNA324938, 2264 |
| DNA324883, 2154 | DNA324939, 2267 |
| DNA324884, 2155 | DNA324940, 2269 |
| DNA324885, 2157 | DNA324941, 2271 |
| DNA324886, 2159 | DNA324942, 2273 |
| DNA324887, 2160 | DNA324943, 2276 |
| DNA324888, 2161 | DNA324944, 2278 |
| DNA324889, 2163 | DNA324945, 2280 |
| DNA324890, 2165 | DNA324946, 2281 |
| DNA324891, 2167 | DNA324947, 2282 |
| DNA324892, 2168 | DNA324948, 2284 |
| DNA324893, 2170 | DNA324949, 2286 |
| DNA324894, 2172 | DNA324950, 2288 |
| DNA324895, 2178 | DNA324951, 2290 |
| DNA324896, 2180 | DNA324952, 2292 |
| DNA324953, 2293 | DNA325010, 2395 |
| DNA324954, 2295 | DNA325011, 2396 |
| DNA324955, 2297 | DNA325012, 2398 |
| DNA324956, 2299 | DNA325013, 2400 |
| DNA324957, 2300 | DNA325014, 2402 |
| DNA324958, 2301 | DNA325015, 2403 |
| DNA324959, 2302 | DNA325016, 2404 |
| DNA324960, 2304 | DNA325017, 2406 |
| DNA324961, 2306 | DNA325018, 2407 |
| DNA324962, 2310 | DNA325019, 2409 |
| DNA324963, 2311 | DNA325020, 2411 |
| DNA324964, 2312 | DNA325021, 2413 |
| DNA324965, 2313 | DNA325022, 2414 |
| DNA324966, 2315 | DNA325023, 2416 |
| DNA324967, 2316 | DNA325024, 2417 |
| DNA324968, 2317 | DNA325025, 2418 |
| DNA324969, 2318 | DNA325026, 2420 |
| DNA324971, 2319 | DNA325027, 2422 |
| DNA324972, 2321 | DNA325028, 2423 |
| DNA324973, 2322 | DNA325029, 2425 |
| DNA324974, 2323 | DNA325030, 2427 |
| DNA324975, 2325 | DNA325031, 2429 |
| DNA324976, 2326 | DNA325032, 2430 |
| DNA324977, 2328 | DNA325033, 2432 |
| DNA324978, 2329 | DNA325034, 2433 |
| DNA324979, 2331 | DNA325035, 2434 |
| DNA324980, 2333 | DNA325036, 2437 |
| DNA324981, 2335 | DNA325037, 2439 |
| DNA324982, 2337 | DNA325038, 2440 |
| DNA324983, 2338 | DNA325039, 2442 |
| DNA324984, 2340 | DNA325040, 2444 |
| DNA324985, 2344 | DNA325041, 2446 |
| DNA324986, 2346 | DNA325042, 2447 |
| DNA324987, 2350 | DNA325043, 2449 |
| DNA324988, 2351 | DNA325044, 2451 |
| DNA324989, 2352 | DNA325045, 2453 |
| DNA324990, 2353 | DNA325046, 2454 |
| DNA324991, 2355 | DNA325047, 2455 |
| DNA324992, 2357 | DNA325048, 2456 |
| DNA324993, 2360 | DNA325049, 2460 |
| DNA324994, 2363 | DNA325050, 2462 |
| DNA324995, 2365 | DNA325051, 2464 |
| DNA324996, 2367 | DNA325052, 2466 |
| DNA324997, 2369 | DNA325053, 2467 |
| DNA324998, 2373 | DNA325054, 2469 |
| DNA324999, 2375 | DNA325055, 2470 |
| DNA325000, 2376 | DNA325056, 2471 |
| DNA325001, 2378 | DNA325057, 2472 |
| DNA325002, 2380 | DNA325058, 2473 |
| DNA325003, 2381 | DNA325059, 2475 |
| DNA325004, 2383 | DNA325060, 2476 |
| DNA325005, 2385 | DNA325061, 2478 |
| DNA325006, 2386 | DNA325062, 2480 |
| DNA325007, 2387 | DNA325063, 2482 |
| DNA325008, 2389 | DNA325064, 2483 |
| DNA325009, 2391 | DNA325065, 2485 |
| DNA325066, 2487 | DNA325122, 2586 |
| DNA325067, 2488 | DNA325123, 2588 |
| DNA325068, 2490 | DNA325124, 2590 |
| DNA325069, 2493 | DNA325125, 2592 |
| DNA325070, 2497 | DNA325126, 2595 |
| DNA325071, 2499 | DNA325127, 2596 |
| DNA325072, 2501 | DNA325128, 2598 |
| DNA325073, 2503 | DNA325129, 2602 |
| DNA325074, 2505 | DNA325130, 2604 |
| DNA325075, 2508 | DNA325131, 2605 |
| DNA325076, 2510 | DNA325132, 2606 |
| DNA325077, 2514 | DNA325133, 2608 |
| DNA325078, 2515 | DNA325134, 2609 |
| DNA325079, 2517 | DNA325135, 2611 |
| DNA325080, 2519 | DNA325136, 2612 |
| DNA325081, 2521 | DNA325137, 2613 |
| DNA325082, 2523 | DNA325138, 2614 |
| DNA325083, 2525 | DNA325139, 2616 |
| DNA325084, 2526 | DNA325140, 2618 |
| DNA325085, 2527 | DNA325141, 2619 |
| DNA325086, 2529 | DNA325143, 2620 |
| DNA325087, 2530 | DNA325144, 2622 |
| DNA325088, 2531 | DNA325145, 2623 |
| DNA325089, 2533 | DNA325146, 2625 |
| DNA325090, 2534 | DNA325147, 2626 |
| DNA325091, 2536 | DNA325148, 2627 |
| DNA325092, 2538 | DNA325149, 2628 |
| DNA325093, 2540 | DNA325150, 2629 |
| DNA325094, 2541 | DNA325151, 2631 |
| DNA325095, 2543 | DNA325152,2633 |
| DNA325096, 2544 | DNA325153, 2635 |
| DNA325097, 2548 | DNA325154, 2637 |
| DNA325098, 2550 | DNA325155, 2638 |
| DNA325099, 2552 | DNA325156, 2640 |
| DNA325100, 2554 | DNA325157, 2641 |
| DNA325101, 2556 | DNA325158, 2642 |
| DNA325102, 2557 | DNA325159, 2644 |
| DNA325103, 2558 | DNA325160, 2645 |
| DNA325104, 2559 | DNA325161, 2646 |
| DNA325105, 2560 | DNA325162, 2647 |
| DNA325106, 2561 | DNA325163,2649 |
| DNA325107, 2562 | DNA325164, 2651 |
| DNA325108, 2563 | DNA325165, 2653 |
| DNA325109, 2564 | DNA325166, 2655 |
| DNA325110, 2567 | DNA325167, 2657 |
| DNA325111, 2569 | DNA325168, 2659 |
| DNA325112, 2571 | DNA325169, 2664 |
| DNA325113, 2572 | DNA325170, 2666 |
| DNA325114, 2574 | DNA325171, 2668 |
| DNA325115, 2575 | DNA325172, 2672 |
| DNA325116, 2577 | DNA325173, 2673 |
| DNA325117, 2579 | DNA325174, 2675 |
| DNA325118, 2581 | DNA325175, 2677 |
| DNA325119, 2582 | DNA325176, 2679 |
| DNA325120, 2583 | DNA325177, 2682 |
| DNA325121, 2584 | DNA325178, 2684 |
| DNA325179, 2686 | DNA325235, 2803 |
| DNA325180, 2688 | DNA325236, 2804 |
| DNA325181, 2689 | DNA325237, 2806 |
| DNA325182, 2697 | DNA325238, 2808 |
| DNA325183, 2699 | DNA325239, 2809 |
| DNA325184, 2700 | DNA325240, 2811 |
| DNA325185, 2705 | DNA325241, 2813 |
| DNA325186, 2707 | DNA325242, 2815 |
| DNA325187, 2708 | DNA325243, 2817 |
| DNA325188, 2710 | DNA325244, 2818 |
| DNA325189, 2711 | DNA325245, 2819 |
| DNA325190, 2712 | DNA325246, 2820 |
| DNA325191, 2716 | DNA325247, 2822 |
| DNA325192, 2718 | DNA325248, 2824 |
| DNA325193, 2720 | DNA325249, 2825 |
| DNA325194, 2722 | DNA325250, 2826 |
| DNA325195, 2725 | DNA325251, 2828 |
| DNA325196, 2726 | DNA325252, 2830 |
| DNA325197, 2727 | DNA325253, 2832 |
| DNA325198, 2728 | DNA325254, 2833 |
| DNA325199, 2730 | DNA325255, 2834 |
| DNA325200, 2732 | DNA325256, 2836 |
| DNA325201, 2736 | DNA325257, 2838 |
| DNA325202, 2738 | DNA325258, 2839 |
| DNA325203, 2742 | DNA325259, 2841 |
| DNA325204, 2744 | DNA325260, 2843 |
| DNA325205, 2748 | DNA325261, 2845 |
| DNA325206, 2750 | DNA325262, 2846 |
| DNA325207, 2753 | DNA325263, 2847 |
| DNA325208, 2755 | DNA325264, 2849 |
| DNA325209, 2756 | DNA325265, 2851 |
| DNA325210, 2757 | DNA325266, 2852 |
| DNA325211, 2759 | DNA325267, 2854 |
| DNA325212, 2760 | DNA325268, 2855 |
| DNA325213, 2765 | DNA325269, 2857 |
| DNA325214, 2766 | DNA325270, 2859 |
| DNA325215, 2769 | DNA325271, 2860 |
| DNA325216, 2771 | DNA325272, 2862 |
| DNA325217, 2772 | DNA325273, 2864 |
| DNA325218, 2774 | DNA325274, 2866 |
| DNA325219, 2775 | DNA325275, 2868 |
| DNA325220, 2777 | DNA325276, 2870 |
| DNA325221, 2778 | DNA325277, 2871 |
| DNA325222, 2780 | DNA325278, 2873 |
| DNA325223, 2784 | DNA325279, 2874 |
| DNA325224, 2786 | DNA325280, 2875 |
| DNA325225, 2787 | DNA325281, 2876 |
| DNA325226, 2789 | DNA325282, 2878 |
| DNA325227, 2790 | DNA325283, 2879 |
| DNA325228, 2792 | DNA325284, 2881 |
| DNA325229, 2794 | DNA325285, 2883 |
| DNA325230, 2798 | DNA325286, 2885 |
| DNA325231, 2799 | DNA325287, 2887 |
| DNA325232, 2800 | DNA325288, 2889 |
| DNA325233, 2801 | DNA325289, 2891 |
| DNA325234, 2802 | DNA325290, 2893 |
| DNA325291, 2895 | DNA325347, 3000 |
| DNA325292, 2897 | DNA325348, 3002 |
| DNA325293, 2898 | DNA325349, 3006 |
| DNA325294, 2901 | DNA325350, 3010 |
| DNA325295, 2902 | DNA325351, 3012 |
| DNA325296, 2904 | DNA325352, 3013 |
| DNA325297, 2906 | DNA325353, 3015 |
| DNA325298, 2908 | DNA325354, 3016 |
| DNA325299, 2909 | DNA325355, 3017 |
| DNA325300, 2910 | DNA325356, 3019 |
| DNA325301, 2911 | DNA325357, 3020 |
| DNA325302, 2913 | DNA325358, 3022 |
| DNA325303, 2914 | DNA325359, 3024 |
| DNA325304, 2916 | DNA325360, 3026 |
| DNA325305, 2918 | DNA325361, 3028 |
| DNA325306, 2919 | DNA325362, 3029 |
| DNA325307, 2921 | DNA325363, 3031 |
| DNA325308, 2922 | DNA325364, 3033 |
| DNA325309, 2923 | DNA325365, 3035 |
| DNA325310, 2925 | DNA325366, 3037 |
| DNA325311, 2926 | DNA325367, 3039 |
| DNA325312, 2927 | DNA325368, 3041 |
| DNA325313, 2929 | DNA325369, 3042 |
| DNA325314, 2930 | DNA325370, 3044 |
| DNA325315, 2931 | DNA325371, 3045 |
| DNA325316, 2933 | DNA325372, 3047 |
| DNA325317, 2934 | DNA325373, 3049 |
| DNA325318, 2935 | DNA325374, 3053 |
| DNA325319, 2937 | DNA325375, 3055 |
| DNA325320, 2939 | DNA325376, 3057 |
| DNA325321, 2941 | DNA325377, 3058 |
| DNA325322, 2942 | DNA325378, 3059 |
| DNA325323, 2944 | DNA325379, 3061 |
| DNA325324, 2945 | DNA325380, 3063 |
| DNA325325, 2949 | DNA325381, 3065 |
| DNA325326, 2953 | DNA325382, 3068 |
| DNA325327, 2955 | DNA325383, 3070 |
| DNA325328, 2957 | DNA325384, 3072 |
| DNA325329, 2959 | DNA325385, 3073 |
| DNA325330, 2963 | DNA325386, 3074 |
| DNA325331, 2966 | DNA325387, 3075 |
| DNA325332, 2968 | DNA325388, 3078 |
| DNA325333, 2970 | DNA325389, 3080 |
| DNA325334, 2971 | DNA325390, 3082 |
| DNA325335, 2973 | DNA325391, 3084 |
| DNA325336, 2975 | DNA325392, 3086 |
| DNA325337, 2976 | DNA325393, 3088 |
| DNA325338, 2977 | DNA325394, 3089 |
| DNA325339, 2978 | DNA325395, 3091 |
| DNA325340, 2980 | DNA325396, 3095 |
| DNA325341, 2984 | DNA325397, 3097 |
| DNA325342, 2988 | DNA325398, 3099 |
| DNA325343, 2992 | DNA325399, 3103 |
| DNA325344, 2994 | DNA325400, 3104 |
| DNA325345, 2998 | DNA325401, 3106 |
| DNA325346, 2999 | DNA325402, 3107 |
| DNA325403, 3111 | DNA325459, 3212 |
| DNA325404, 3115 | DNA325460, 3214 |
| DNA325405, 3117 | DNA325461, 3217 |
| DNA325406, 3119 | DNA325462, 3222 |
| DNA325407, 3120 | DNA325463, 3223 |
| DNA325408, 3122 | DNA325464, 3224 |
| DNA325409, 3124 | DNA325465, 3225 |
| DNA325410, 3125 | DNA325466, 3227 |
| DNA325411, 3127 | DNA325467, 3228 |
| DNA325412, 3129 | DNA325468, 3230 |
| DNA325413, 3131 | DNA325469, 3232 |
| DNA325414, 3133 | DNA325470, 3234 |
| DNA325415, 3135 | DNA325471, 3238 |
| DNA325416, 3136 | DNA325472, 3240 |
| DNA325417, 3137 | DNA325473, 3242 |
| DNA325418, 3139 | DNA325474, 3244 |
| DNA325419, 3141 | DNA325475, 3247 |
| DNA325420, 3142 | DNA325476, 3248 |
| DNA325421, 3144 | DNA325477, 3249 |
| DNA325422, 3146 | DNA325478, 3251 |
| DNA325423, 3148 | DNA325479, 3253 |
| DNA325424, 3149 | DNA325480, 3255 |
| DNA325425, 3151 | DNA325481, 3256 |
| DNA325426, 3152 | DNA325482, 3258 |
| DNA325427, 3153 | DNA325483, 3260 |
| DNA325428, 3155 | DNA325484, 3261 |
| DNA325429, 3157 | DNA325485, 3263 |
| DNA325430, 3159 | DNA325486, 3264 |
| DNA325431, 3161 | DNA325487, 3266 |
| DNA325432, 3163 | DNA325488, 3268 |
| DNA325433, 3165 | DNA325489, 3269 |
| DNA325434, 3167 | DNA325490, 3270 |
| DNA325435, 3169 | DNA325491, 3271 |
| DNA325436, 3170 | DNA325492, 3273 |
| DNA325437, 3171 | DNA325493, 3275 |
| DNA325438, 3173 | DNA325494, 3276 |
| DNA325439, 3177 | DNA325495, 3278 |
| DNA325440, 3178 | DNA325496, 3279 |
| DNA325441, 3180 | DNA325497, 3281 |
| DNA325442, 3182 | DNA325498, 3283 |
| DNA325443, 3183 | DNA325499, 3286 |
| DNA325444, 3184 | DNA325500, 3287 |
| DNA325445, 3185 | DNA325501, 3288 |
| DNA325446, 3187 | DNA325502, 3289 |
| DNA325447, 3188 | DNA325503, 3291 |
| DNA325448, 3190 | DNA325504, 3293 |
| DNA325449, 3192 | DNA325505, 3294 |
| DNA325450, 3193 | DNA325506, 3299 |
| DNA325451, 3194 | DNA325507, 3301 |
| DNA325452, 3195 | DNA325508, 3303 |
| DNA325453, 3196 | DNA325509, 3304 |
| DNA325454, 3197 | DNA325510, 3306 |
| DNA325455, 3199 | DNA325511, 3308 |
| DNA325456, 3201 | DNA325512, 3310 |
| DNA325457, 3202 | DNA325513, 3311 |
| DNA325458, 3210 | DNA325514, 3315 |
| DNA325515, 3316 | DNA325571, 3417 |
| DNA325516, 3318 | DNA325572, 3418 |
| DNA325517, 3320 | DNA325573, 3420 |
| DNA325518, 3322 | DNA325574, 3422 |
| DNA325519, 3324 | DNA325575, 3424 |
| DNA325520, 3325 | DNA325576, 3426 |
| DNA325521, 3326 | DNA325577, 3427 |
| DNA325522, 3328 | DNA325578, 3428 |
| DNA325523, 3331 | DNA325579, 3429 |
| DNA325524, 3335 | DNA325580, 3430 |
| DNA325525, 3336 | DNA325581, 3432 |
| DNA325526, 3337 | DNA325582, 3436 |
| DNA325527, 3339 | DNA325583, 3437 |
| DNA325528, 3341 | DNA325584, 3439 |
| DNA325529, 3342 | DNA325585, 3441 |
| DNA325530, 3344 | DNA325586, 3442 |
| DNA325531, 3346 | DNA325587, 3444 |
| DNA325532, 3348 | DNA325588, 3446 |
| DNA325533, 3349 | DNA325589, 3448 |
| DNA325534, 3350 | DNA325590, 3450 |
| DNA325535, 3352 | DNA325591, 3451 |
| DNA325536, 3353 | DNA325592, 3454 |
| DNA325537, 3355 | DNA325593, 3455 |
| DNA325538, 3357 | DNA325594, 3457 |
| DNA325539, 3358 | DNA325595, 3458 |
| DNA325540, 3359 | DNA325596, 3460 |
| DNA325541, 3361 | DNA325597, 3462 |
| DNA325542, 3363 | DNA325598, 3463 |
| DNA325543, 3364 | DNA325599, 3465 |
| DNA325544, 3365 | DNA325600, 3470 |
| DNA325545, 3366 | DNA325601, 3472 |
| DNA325546, 3367 | DNA325602, 3475 |
| DNA325547, 3369 | DNA325603, 3482 |
| DNA325548, 3371 | DNA325604, 3483 |
| DNA325549, 3373 | DNA325605, 3485 |
| DNA325550, 3374 | DNA325606, 3486 |
| DNA325551, 3378 | DNA325607, 3488 |
| DNA325552, 3382 | DNA325608, 3491 |
| DNA325553, 3384 | DNA325609, 3493 |
| DNA325554, 3386 | DNA325610, 3494 |
| DNA325555, 3388 | DNA325611, 3495 |
| DNA325556, 3394 | DNA325612, 3496 |
| DNA325557, 3395 | DNA325613, 3500 |
| DNA325558, 3397 | DNA325614, 3501 |
| DNA325559, 3398 | DNA325615, 3503 |
| DNA325560, 3399 | DNA325616, 3504 |
| DNA325561, 3400 | DNA325617, 3506 |
| DNA325562, 3401 | DNA325618, 3507 |
| DNA325563, 3403 | DNA325619, 3509 |
| DNA325564, 3404 | DNA325620, 3513 |
| DNA325565, 3406 | DNA325621, 3515 |
| DNA325566, 3407 | DNA325622, 3517 |
| DNA325567, 3409 | DNA325623, 3519 |
| DNA325568, 3411 | DNA325624, 3522 |
| DNA325569, 3413 | DNA325625, 3528 |
| DNA325570, 3414 | DNA325626, 3529 |
| DNA325627, 3531 | DNA325683, 3634 |
| DNA325628, 3532 | DNA325684, 3635 |
| DNA325629, 3533 | DNA325685, 3636 |
| DNA325630, 3535 | DNA325686, 3638 |
| DNA325631, 3536 | DNA325687, 3640 |
| DNA325632, 3538 | DNA325688, 3641 |
| DNA325633, 3539 | DNA325689, 3642 |
| DNA325634, 3540 | DNA325690, 3643 |
| DNA325635, 3542 | DNA325691, 3645 |
| DNA325636, 3543 | DNA325692, 3646 |
| DNA325637, 3545 | DNA325693, 3648 |
| DNA325638, 3546 | DNA325694, 3650 |
| DNA325639, 3548 | DNA325695, 3652 |
| DNA325640, 3552 | DNA325696, 3654 |
| DNA325641, 3554 | DNA325697, 3656 |
| DNA325642, 3557 | DNA325698, 3658 |
| DNA325643, 3559 | DNA325699, 3659 |
| DNA325644, 3560 | DNA325700, 3660 |
| DNA325645, 3561 | DNA325701, 3662 |
| DNA325646, 3562 | DNA325702, 3663 |
| DNA325647, 3564 | DNA325703, 3665 |
| DNA325648, 3566 | DNA325704, 3669 |
| DNA325649, 3568 | DNA325705, 3671 |
| DNA325650, 3570 | DNA325706, 3672 |
| DNA325651, 3571 | DNA325707, 3674 |
| DNA325652, 3572 | DNA325708, 3676 |
| DNA325653, 3574 | DNA325709, 3680 |
| DNA325654, 3576 | DNA325710, 3681 |
| DNA325655, 3578 | DNA325711, 3683 |
| DNA325656, 3579 | DNA325712, 3685 |
| DNA325657, 3580 | DNA325713, 3687 |
| DNA325658, 3581 | DNA325714, 3689 |
| DNA325659, 3582 | DNA325715, 3691 |
| DNA325660, 3583 | DNA325716, 3693 |
| DNA325661, 3584 | DNA325717, 3695 |
| DNA325662, 3585 | DNA325718, 3697 |
| DNA325663, 3586 | DNA325719, 3699 |
| DNA325664, 3590 | DNA325720, 3700 |
| DNA325665, 3595 | DNA325721, 3702 |
| DNA325666, 3596 | DNA325722, 3704 |
| DNA325667, 3598 | DNA325723, 3705 |
| DNA325668, 3599 | DNA325724, 3707 |
| DNA325669, 3602 | DNA325725, 3708 |
| DNA325670, 3604 | DNA325726, 3710 |
| DNA325671, 3606 | DNA325727, 3712 |
| DNA325672, 3608 | DNA325728, 3714 |
| DNA325673, 3610 | DNA325729, 3715 |
| DNA325674, 3612 | DNA325730, 3719 |
| DNA325675, 3614 | DNA325731, 3722 |
| DNA325676, 3616 | DNA325732, 3726 |
| DNA325677, 3618 | DNA325733, 3731 |
| DNA325678, 3622 | DNA325734, 3732 |
| DNA325679, 3624 | DNA325736, 3734 |
| DNA325680, 3626 | DNA3 25737, 3736 |
| DNA325681, 3630 | DNA325738, 3737 |
| DNA325682, 3633 | DNA325739, 3739 |
| DNA325740, 3740 | DNA325797, 3841 |
| DNA325741, 3742 | DNA325798, 3843 |
| DNA325742, 3744 | DNA325799, 3845 |
| DNA325743, 3746 | DNA325800, 3847 |
| DNA325744, 3748 | DNA325801, 3849 |
| DNA325745, 3750 | DNA325802, 3851 |
| DNA325746, 3752 | DNA325803, 3853 |
| DNA325747, 3754 | DNA325804, 3855 |
| DNA325748, 3755 | DNA325805, 3856 |
| DNA325749, 3757 | DNA325806, 3857 |
| DNA325750, 3759 | DNA325807, 3859 |
| DNA325751, 3761 | DNA325808, 3861 |
| DNA325752, 3765 | DNA325809, 3862 |
| DNA325753, 3766 | DNA325810, 3868 |
| DNA325754, 3767 | DNA325811, 3869 |
| DNA325755, 3769 | DNA325812, 3870 |
| DNA325756, 3771 | DNA325813, 3872 |
| DNA325757, 3772 | DNA325814, 3874 |
| DNA325758, 3773 | DNA325815, 3876 |
| DNA325759, 3774 | DNA325816, 3877 |
| DNA325760, 3775 | DNA325817, 3878 |
| DNA325761, 3779 | DNA325818, 3880 |
| DNA325762, 3781 | DNA325819, 3881 |
| DNA325763, 3783 | DNA325820, 3883 |
| DNA325764, 3785 | DNA325821, 3884 |
| DNA325765, 3787 | DNA325822, 3886 |
| DNA325766, 3788 | DNA325823, 3889 |
| DNA325767, 3790 | DNA325824, 3891 |
| DNA325768, 3792 | DNA325825, 3893 |
| DNA325769, 3794 | DNA325826, 3895 |
| DNA325770, 3796 | DNA325827, 3898 |
| DNA325771, 3797 | DNA325828, 3902 |
| DNA325772, 3798 | DNA325829, 3903 |
| DNA325773, 3800 | DNA325830, 3904 |
| DNA325775, 3802 | DNA325831, 3906 |
| DNA325776, 3804 | DNA325832, 3908 |
| DNA325777, 3805 | DNA325833, 3910 |
| DNA325778, 3807 | DNA325834, 3914 |
| DNA325779, 3809 | DNA325835, 3916 |
| DNA325780, 3810 | DNA325836, 3917 |
| DNA325781, 3812 | DNA325837, 3918 |
| DNA325782, 3814 | DNA325838, 3920 |
| DNA325783, 3816 | DNA325839, 3921 |
| DNA325784, 3818 | DNA325840, 3923 |
| DNA325785, 3819 | DNA325841, 3924 |
| DNA325786, 3821 | DNA325842, 3925 |
| DNA325787, 3825 | DNA325843, 3926 |
| DNA325788, 3826 | DNA325844, 3928 |
| DNA325789, 3829 | DNA325845, 3930 |
| DNA325790, 3831 | DNA325847, 3931 |
| DNA325791, 3833 | DNA325848, 3932 |
| DNA325792, 3834 | DNA325849, 3933 |
| DNA325793, 3835 | DNA325850, 3935 |
| DNA325794, 3836 | DNA325851, 3937 |
| DNA325795, 3837 | DNA325852, 3938 |
| DNA325796, 3839 | DNA325853, 3940 |
| DNA325854, 3942 | DNA325910, 4037 |
| DNA325855, 3944 | DNA325911, 4039 |
| DNA325856, 3946 | DNA325912, 4040 |
| DNA325857, 3948 | DNA325913, 4044 |
| DNA325858, 3949 | DNA325914, 4045 |
| DNA325859, 3950 | DNA325915, 4046 |
| DNA325860, 3951 | DNA325916, 4048 |
| DNA325861, 3953 | DNA325917, 4050 |
| DNA325862, 3955 | DNA325918, 4052 |
| DNA325863, 3957 | DNA325919, 4054 |
| DNA325864, 3958 | DNA325920, 4055 |
| DNA325865, 3959 | DNA325921, 4057 |
| DNA325866, 3960 | DNA325922, 4061 |
| DNA325867, 3964 | DNA325923, 4063 |
| DNA325868, 3966 | DNA325924, 4065 |
| DNA325869, 3967 | DNA325925, 4067 |
| DNA325870, 3968 | DNA325926, 4068 |
| DNA325871, 3969 | DNA325927, 4069 |
| DNA325872, 3971 | DNA325928, 4071 |
| DNA325873, 3973 | DNA325929, 4072 |
| DNA325874, 3975 | DNA325930, 4073 |
| DNA325875, 3978 | DNA325931, 4074 |
| DNA325876, 3980 | DNA325932, 4075 |
| DNA325877, 3981 | DNA325933, 4077 |
| DNA325878, 3983 | DNA325934, 4081 |
| DNA325879, 3986 | DNA325935, 4082 |
| DNA325880, 3987 | DNA325936, 4084 |
| DNA325881, 3988 | DNA325937, 4086 |
| DNA325882, 3990 | DNA325938, 4088 |
| DNA325883, 3991 | DNA325939, 4090 |
| DNA325884, 3994 | DNA325940, 4091 |
| DNA325885, 3996 | DNA325941, 4092 |
| DNA325886, 3997 | DNA325942, 4094 |
| DNA325887, 3999 | DNA325943, 4097 |
| DNA325888, 4001 | DNA325944, 4098 |
| DNA325889, 4003 | DNA325945, 4100 |
| DNA325890, 4005 | DNA325946, 4101 |
| DNA325891, 4006 | DNA325947, 4103 |
| DNA325892, 4008 | DNA325948, 4105 |
| DNA325893, 4010 | DNA325949, 4106 |
| DNA325894, 4012 | DNA325950, 4108 |
| DNA325895, 4014 | DNA325951, 4112 |
| DNA325896, 4016 | DNA325952, 4114 |
| DNA325897, 4018 | DNA325953, 4115 |
| DNA325898, 4019 | DNA325954, 4116 |
| DNA325899, 4020 | DNA325955, 4118 |
| DNA325900, 4022 | DNA325956, 4119 |
| DNA325901, 4024 | DNA325957, 4120 |
| DNA325902, 4025 | DNA325958, 4121 |
| DNA325903, 4027 | DNA325959, 4122 |
| DNA325904, 4029 | DNA325960, 4123 |
| DNA325905, 4031 | DNA325961, 4124 |
| DNA325906, 4032 | DNA325962, 4125 |
| DNA325907, 4033 | DNA325963, 4127 |
| DNA325908, 4034 | DNA325964, 4129 |
| DNA325909, 4035 | DNA325965, 4130 |
| DNA325966, 4132 | DNA326022, 4239 |
| DNA325967, 4133 | DNA326023, 4241 |
| DNA325968, 4134 | DNA326024, 4244 |
| DNA325969, 4135 | DNA326025, 4245 |
| DNA325970, 4136 | DNA326026, 4246 |
| DNA325971, 4138 | DNA326027, 4248 |
| DNA325972, 4139 | DNA326028, 4250 |
| DNA325973, 4143 | DNA326029, 4251 |
| DNA325974, 4145 | DNA326030, 4252 |
| DNA325975, 4147 | DNA326031, 4254 |
| DNA325976, 4148 | DNA326032, 4256 |
| DNA325977, 4150 | DNA326033, 4257 |
| DNA325978, 4152 | DNA326034, 4259 |
| DNA325979, 4154 | DNA326035, 4261 |
| DNA325980, 4156 | DNA326036, 4263 |
| DNA325981, 4157 | DNA326037, 4269 |
| DNA325982, 4159 | DNA326038, 4270 |
| DNA325983, 4160 | DNA326039, 4272 |
| DNA325984, 4163 | DNA326040, 4273 |
| DNA325985, 4165 | DNA326041, 4275 |
| DNA325986, 4167 | DNA326042, 4277 |
| DNA325987, 4168 | DNA326043, 4278 |
| DNA325988, 4172 | DNA326044, 4279 |
| DNA325989, 4174 | DNA326045, 4281 |
| DNA325990, 4176 | DNA326046, 4282 |
| DNA325991, 4178 | DNA326047, 4283 |
| DNA325992, 4180 | DNA326048, 4285 |
| DNA325993, 4184 | DNA326049, 4286 |
| DNA325994, 4186 | DNA326050, 4287 |
| DNA325995, 4187 | DNA326051, 4289 |
| DNA325996, 4189 | DNA326052, 4290 |
| DNA325997, 4191 | DNA326053, 4292 |
| DNA325998, 4193 | DNA326054, 4293 |
| DNA325999, 4195 | DNA326055, 4295 |
| DNA326000, 4197 | DNA326056, 4296 |
| DNA326001, 4199 | DNA326057, 4298 |
| DNA326002, 4200 | DNA326058, 4302 |
| DNA326003, 4202 | DNA326059, 4304 |
| DNA326004, 4203 | DNA326060, 4307 |
| DNA326005, 4205 | DNA326061, 4309 |
| DNA326006, 4207 | DNA326062, 4310 |
| DNA326007, 4210 | DNA326063, 4311 |
| DNA326008, 4211 | DNA326064, 4312 |
| DNA326009, 4213 | DNA326065, 4314 |
| DNA326010, 4216 | DNA326066, 4315 |
| DNA326011, 4218 | DNA326067, 4317 |
| DNA326012, 4220 | DNA326068, 4319 |
| DNA326013, 4221 | DNA326069, 4322 |
| DNA326014, 4222 | DNA326070, 4323 |
| DNA326015, 4226 | DNA326071, 4325 |
| DNA326016, 4228 | DNA326072, 4326 |
| DNA326017, 4230 | DNA326073, 4327 |
| DNA326018, 4232 | DNA326074, 4329 |
| DNA326019, 4234 | DNA326075, 4331 |
| DNA326020, 4236 | DNA326076, 4333 |
| DNA326021, 4237 | DNA326077, 4334 |
| DNA326078, 4335 | DNA326134, 4444 |
| DNA326079, 4337 | DNA326135, 4448 |
| DNA326080, 4338 | DNA326136, 4449 |
| DNA326081, 4340 | DNA326137, 4451 |
| DNA326082, 4342 | DNA326138, 4453 |
| DNA326083, 4344 | DNA326139, 4454 |
| DNA326084, 4346 | DNA326140, 4456 |
| DNA326085, 4348 | DNA326141, 4458 |
| DNA326086, 4350 | DNA326142, 4460 |
| DNA326087, 4352 | DNA326143, 4461 |
| DNA326088, 4353 | DNA326144, 4462 |
| DNA326089, 4354 | DNA326145, 4463 |
| DNA326090, 4356 | DNA326146,4465 |
| DNA326091, 4358 | DNA326147, 4467 |
| DNA326092, 4364 | DNA326148,4468 |
| DNA326093, 4366 | DNA326149, 4470 |
| DNA326094, 4368 | DNA326150, 4472 |
| DNA326095, 4372 | DNA326151, 4474 |
| DNA326096, 4376 | DNA326152, 4478 |
| DNA326097, 4378 | DNA326153, 4479 |
| DNA326098, 4380 | DNA326154, 4480 |
| DNA326099, 4382 | DNA326155, 4482 |
| DNA326100, 4384 | DNA326156, 4483 |
| DNA326101, 4386 | DNA326157, 4484 |
| DNA326102, 4388 | DNA326158, 4485 |
| DNA326103, 4390 | DNA326159, 4489 |
| DNA326104, 4392 | DNA326160, 4490 |
| DNA326105, 4394 | DNA326161, 4491 |
| DNA326106, 4396 | DNA326162, 4493 |
| DNA326107, 4398 | DNA326163, 4495 |
| DNA326108, 4400 | DNA326164, 4497 |
| DNA326109, 4402 | DNA326165, 4498 |
| DNA326110, 4404 | DNA326166, 4500 |
| DNA326111, 4406 | DNA326167, 4502 |
| DNA326112, 4408 | DNA326168, 4504 |
| DNA326113, 4410 | DNA326169, 4505 |
| DNA326114, 4411 | DNA326170, 4509 |
| DNA326115, 4413 | DNA326171, 4511 |
| DNA326116, 4414 | DNA326172, 4513 |
| DNA326117, 4416 | DNA326173, 4514 |
| DNA326118, 4418 | DNA326174, 4518 |
| DNA326119, 4420 | DNA326175, 4522 |
| DNA326120, 4423 | DNA326176, 4524 |
| DNA326121, 4425 | DNA326177, 4526 |
| DNA326122, 4426 | DNA326178, 4527 |
| DNA326123, 4427 | DNA326179, 4528 |
| DNA326124, 4429 | DNA326180, 4532 |
| DNA326125, 4430 | DNA326181, 4534 |
| DNA326126, 4431 | DNA326182, 4535 |
| DNA326127, 4432 | DNA326183, 4537 |
| DNA326128, 4434 | DNA326184, 4538 |
| DNA326129, 4435 | DNA326185, 4539 |
| DNA326130, 4436 | DNA326186, 4541 |
| DNA326131, 4438 | DNA326187, 4543 |
| DNA326132, 4440 | DNA326188, 4544 |
| DNA326133, 4442 | DNA326189, 4545 |
| DNA326190, 4547 | DNA326246, 4651 |
| DNA326191, 4549 | DNA326247, 4653 |
| DNA326192, 4551 | DNA326248, 4654 |
| DNA326193, 4553 | DNA326249, 4656 |
| DNA326194, 4555 | DNA326250, 4658 |
| DNA326195, 4556 | DNA326251, 4659 |
| DNA326196, 4558 | DNA326252, 4661 |
| DNA326197, 4560 | DNA326253, 4663 |
| DNA326198, 4561 | DNA326254, 4665 |
| DNA326199, 4562 | DNA326255, 4667 |
| DNA326200, 4566 | DNA326256, 4669 |
| DNA326201, 4570 | DNA326257, 4671 |
| DNA326202, 4571 | DNA326258, 4672 |
| DNA326203, 4573 | DNA326259, 4674 |
| DNA326204, 4577 | DNA326260, 4675 |
| DNA326205, 4581 | DNA326261, 4677 |
| DNA326206, 4583 | DNA326262, 4678 |
| DNA326207, 4584 | DNA326263, 4680 |
| DNA326208, 4586 | DNA326264, 4682 |
| DNA326209, 4588 | DNA326265, 4684 |
| DNA326210, 4590 | DNA326266, 4686 |
| DNA326211, 4592 | DNA326267, 4689 |
| DNA326212, 4594 | DNA326268, 4691 |
| DNA326213, 4596 | DNA326269, 4693 |
| DNA326214, 4597 | DNA326270, 4694 |
| DNA326215, 4599 | DNA326271, 4695 |
| DNA326216, 4600 | DNA326272, 4696 |
| DNA326217, 4602 | DNA326273, 4697 |
| DNA326218, 4604 | DNA326274, 4701 |
| DNA326219, 4606 | DNA326275, 4703 |
| DNA326220, 4608 | DNA326276, 4704 |
| DNA326221, 4610 | DNA326277, 4706 |
| DNA326222, 4612 | DNA326278, 4707 |
| DNA326223, 4614 | DNA326279, 4710 |
| DNA326224, 4616 | DNA326280, 4712 |
| DNA326225, 4617 | DNA326281, 4713 |
| DNA326226, 4619 | DNA326282, 4716 |
| DNA326227, 4621 | DNA326283, 4718 |
| DNA326228, 4622 | DNA326284, 4721 |
| DNA326229, 4624 | DNA326285, 4723 |
| DNA326230, 4628 | DNA326286, 4724 |
| DNA326231, 4630 | DNA326287, 4725 |
| DNA326232, 4632 | DNA326288, 4727 |
| DNA326233, 4633 | DNA326289, 4730 |
| DNA326234, 4635 | DNA326290, 4732 |
| DNA326235, 4637 | DNA326291, 4734 |
| DNA326236, 4638 | DNA326292, 4735 |
| DNA326237, 4640 | DNA326293, 4737 |
| DNA326238, 4641 | DNA326294, 4739 |
| DNA326239, 4642 | DNA326295, 4741 |
| DNA326240, 4644 | DNA326296, 4744 |
| DNA326241, 4645 | DNA326297, 4745 |
| DNA326242, 4646 | DNA326298, 4749 |
| DNA326243, 4647 | DNA326299, 4750 |
| DNA326244, 4648 | DNA326300, 4751 |
| DNA326245, 4650 | DNA326301, 4752 |
| DNA326302, 4754 | DNA326358, 4867 |
| DNA326303, 4755 | DNA326359, 4869 |
| DNA326304, 4757 | DNA326360, 4871 |
| DNA326305, 4758 | DNA326361, 4873 |
| DNA326306, 4760 | DNA326362, 4875 |
| DNA326307, 4761 | DNA326363, 4880 |
| DNA326308, 4763 | DNA326364, 4881 |
| DNA326309, 4765 | DNA326365, 4883 |
| DNA326310, 4767 | DNA326366, 4885 |
| DNA326311, 4768 | DNA326367, 4893 |
| DNA326312, 4769 | DNA326368, 4895 |
| DNA326313, 4771 | DNA326369, 4897 |
| DNA326314, 4773 | DNA326370, 4902 |
| DNA326315, 4775 | DNA326371, 4905 |
| DNA326316, 4777 | DNA326372, 4906 |
| DNA326317, 4780 | DNA326373, 4908 |
| DNA326318, 4784 | DNA326374, 4910 |
| DNA326319, 4786 | DNA326375, 4911 |
| DNA326320, 4788 | DNA326376, 4913 |
| DNA326321, 4790 | DNA326377, 4915 |
| DNA326322, 4792 | DNA326378, 4916 |
| DNA326323, 4794 | DNA326379, 4917 |
| DNA326324, 4798 | DNA326380, 4921 |
| DNA326325, 4800 | DNA326381, 4923 |
| DNA326326, 4801 | DNA326382, 4924 |
| DNA326327, 4803 | DNA326383, 4926 |
| DNA326328, 4807 | DNA326384, 4927 |
| DNA326329, 4809 | DNA326385, 4929 |
| DNA326330, 4810 | DNA326386, 4931 |
| DNA326331, 4814 | DNA326387, 4933 |
| DNA326332, 4816 | DNA326388, 4935 |
| DNA326333, 4818 | DNA326389, 4938 |
| DNA326334, 4819 | DNA326390, 4941 |
| DNA326335, 4822 | DNA326391, 4942 |
| DNA326336, 4824 | DNA326392, 4943 |
| DNA326337, 4825 | DNA326393, 4944 |
| DNA326338, 4826 | DNA326394, 4945 |
| DNA326339, 4827 | DNA326395, 4946 |
| DNA326340, 4829 | DNA326396, 4948 |
| DNA326341, 4830 | DNA326397, 4950 |
| DNA326342, 4832 | DNA326398, 4951 |
| DNA326343, 4834 | DNA326399, 4955 |
| DNA326344, 4836 | DNA326400, 4957 |
| DNA326345, 4838 | DNA326401, 4958 |
| DNA326346, 4840 | DNA326402, 4960 |
| DNA326347, 4847 | DNA326403, 4962 |
| DNA326348, 4849 | DNA326404, 4965 |
| DNA326349, 4850 | DNA326405, 4967 |
| DNA326350, 4852 | DNA326406, 4969 |
| DNA326351, 4856 | DNA326407, 4971 |
| DNA326352, 4857 | DNA326408, 4973 |
| DNA326353, 4859 | DNA326409, 4977 |
| DNA326354, 4861 | DNA326410, 4978 |
| DNA326355, 4863 | DNA326411, 4980 |
| DNA326356, 4864 | DNA326412, 4982 |
| DNA326357, 4865 | DNA326413, 4984 |
| DNA326414, 4987 | DNA326470, 5078 |
| DNA326415, 4988 | DNA326471, 5080 |
| DNA326416, 4989 | DNA326472, 5082 |
| DNA326417, 4991 | DNA326473, 5083 |
| DNA326418, 4992 | DNA326474, 5084 |
| DNA326419, 4994 | DNA326475, 5086 |
| DNA326420, 4995 | DNA326476, 5088 |
| DNA326421, 4996 | DNA326477, 5089 |
| DNA326422, 4998 | DNA326478, 5090 |
| DNA326423, 4999 | DNA326479, 5092 |
| DNA326424, 5000 | DNA326480, 5093 |
| DNA326425, 5001 | DNA326481, 5095 |
| DNA326426, 5002 | DNA326482, 5097 |
| DNA326427, 5004 | DNA326483, 5098 |
| DNA326428, 5006 | DNA326484, 5100 |
| DNA326429, 5008 | DNA326485, 5102 |
| DNA326430, 5010 | DNA326486, 5104 |
| DNA326431, 5011 | DNA326487, 5106 |
| DNA326432, 5013 | DNA326488, 5108 |
| DNA326433, 5016 | DNA326489, 5109 |
| DNA326434, 5018 | DNA326490, 5110 |
| DNA326435, 5019 | DNA326491, 5112 |
| DNA326436, 5020 | DNA326492, 5113 |
| DNA326437, 5021 | DNA326493, 5114 |
| DNA326438, 5022 | DNA326494, 5117 |
| DNA326439, 5025 | DNA326495, 5119 |
| DNA326440, 5026 | DNA326496, 5120 |
| DNA326441, 5027 | DNA326497, 5122 |
| DNA326442, 5028 | DNA326498, 5124 |
| DNA326443, 5030 | DNA326499, 5126 |
| DNA326444, 5031 | DNA326500, 5128 |
| DNA326445, 5032 | DNA326501, 5130 |
| DNA326446, 5034 | DNA326502, 5131 |
| DNA326447, 5036 | DNA326503, 5132 |
| DNA326448, 5037 | DNA326504, 5134 |
| DNA326449, 5042 | DNA326505, 5135 |
| DNA326450, 5043 | DNA326506, 5137 |
| DNA326451, 5045 | DNA326507, 5138 |
| DNA326452, 5046 | DNA326508, 5140 |
| DNA326453, 5048 | DNA326509, 5141 |
| DNA326454, 5049 | DNA326510, 5143 |
| DNA326455, 5054 | DNA326511, 5145 |
| DNA326456, 5055 | DNA326512, 5148 |
| DNA326457, 5058 | DNA326513, 5150 |
| DNA326458, 5060 | DNA326514, 5152 |
| DNA326459, 5062 | DNA326515, 5155 |
| DNA326460, 5064 | DNA326516, 5157 |
| DNA326461, 5065 | DNA326517, 5159 |
| DNA326462, 5066 | DNA326518, 5160 |
| DNA326463, 5067 | DNA326519, 5161 |
| DNA326464, 5069 | DNA326520, 5163 |
| DNA326465, 5071 | DNA326521, 5165 |
| DNA326466, 5072 | DNA326522, 5166 |
| DNA326467, 5074 | DNA326523, 5168 |
| DNA326468, 5075 | DNA326524, 5169 |
| DNA326469, 5076 | DNA326525, 5171 |
| DNA326526, 5173 | DNA326583, 5270 |
| DNA326527, 5175 | DNA326584, 5274 |
| DNA326528, 5176 | DNA326585, 5276 |
| DNA326529, 5178 | DNA326586, 5281 |
| DNA326530, 5180 | DNA326587, 5283 |
| DNA326531, 5181 | DNA326588, 5285 |
| DNA326532, 5183 | DNA326589, 5286 |
| DNA326533, 5184 | DNA326590, 5288 |
| DNA326534, 5186 | DNA326591, 5290 |
| DNA326535, 5188 | DNA326592, 5292 |
| DNA326536, 5190 | DNA326593, 5294 |
| DNA326537, 5192 | DNA326594, 5295 |
| DNA326538, 5194 | DNA326595, 5297 |
| DNA326539, 5195 | DNA326596, 5300 |
| DNA326540, 5196 | DNA326597, 5302 |
| DNA326541, 5197 | DNA326598, 5303 |
| DNA326542, 5203 | DNA326599, 5305 |
| DNA326543, 5205 | DNA326600, 5307 |
| DNA326544, 5208 | DNA326601, 5308 |
| DNA326546, 5210 | DNA326602, 5310 |
| DNA326547, 5212 | DNA326603, 5311 |
| DNA326548, 5213 | DNA326604, 5314 |
| DNA326549, 5214 | DNA326605, 5316 |
| DNA326550, 5216 | DNA326606, 5317 |
| DNA326551, 5218 | DNA326607, 5319 |
| DNA326552, 5219 | DNA326608, 5321 |
| DNA326553, 5221 | DNA326609, 5323 |
| DNA326554, 5222 | DNA326610, 5325 |
| DNA326555, 5223 | DNA326611, 5326 |
| DNA326556, 5225 | DNA326612, 5330 |
| DNA326557, 5226 | DNA326613, 5331 |
| DNA326558, 5227 | DNA326614, 5332 |
| DNA326559, 5229 | DNA326615, 5334 |
| DNA326560, 5230 | DNA326616, 5336 |
| DNA326561, 5236 | DNA326617, 5337 |
| DNA326562, 5237 | DNA326618, 5338 |
| DNA326563, 5239 | DNA326619, 5339 |
| DNA326564, 5240 | DNA326620, 5341 |
| DNA326565, 5241 | DNA326621, 5343 |
| DNA326566, 5243 | DNA326622, 5345 |
| DNA326567, 5244 | DNA326623, 5347 |
| DNA326568, 5246 | DNA326624, 5349 |
| DNA326569, 5247 | DNA326625, 5350 |
| DNA326570, 5248 | DNA326626, 5354 |
| DNA326571, 5250 | DNA326627, 5355 |
| DNA326572, 5252 | DNA326628, 5357 |
| DNA326573, 5254 | DNA326629, 5358 |
| DNA326574, 5256 | DNA326630, 5360 |
| DNA326575, 5257 | DNA326631, 5362 |
| DNA326576, 5260 | DNA326632, 5364 |
| DNA326577, 5261 | DNA326633, 5366 |
| DNA326578, 5262 | DNA326634, 5367 |
| DNA326579, 5264 | DNA326635, 5369 |
| DNA326580, 5266 | DNA326636, 5370 |
| DNA326581, 5267 | DNA326637, 5371 |
| DNA326582, 5269 | DNA326638, 5372 |
| DNA326639, 5374 | DNA326695, 5474 |
| DNA326640, 5376 | DNA326696, 5478 |
| DNA326641, 5378 | DNA326697, 5480 |
| DNA326642, 5379 | DNA326698, 5482 |
| DNA326643, 5380 | DNA326699, 5483 |
| DNA326644, 5382 | DNA326700, 5484 |
| DNA326645, 5383 | DNA326701, 5485 |
| DNA326646, 5384 | DNA326702, 5486 |
| DNA326647, 5385 | DNA326703, 5487 |
| DNA326648, 5389 | DNA326704, 5488 |
| DNA326649, 5391 | DNA326705, 5489 |
| DNA326650, 5393 | DNA326706, 5491 |
| DNA326651, 5395 | DNA326707, 5492 |
| DNA326652, 5396 | DNA326708, 5496 |
| DNA326653, 5398 | DNA326709, 5497 |
| DNA326654, 5399 | DNA326710, 5499 |
| DNA326655, 5401 | DNA326711, 5501 |
| DNA326656, 5403 | DNA326712, 5508 |
| DNA326657, 5404 | DNA326713, 5510 |
| DNA326658, 5406 | DNA326714, 5519 |
| DNA326659, 5408 | DNA326715, 5521 |
| DNA326660, 5409 | DNA326716, 5522 |
| DNA326661, 5411 | DNA326717, 5525 |
| DNA326662, 5413 | DNA326718, 5527 |
| DNA326663, 5415 | DNA326719, 5528 |
| DNA326664, 5417 | DNA326720, 5529 |
| DNA326665, 5419 | DNA326721, 5530 |
| DNA326666, 5423 | DNA326722, 5531 |
| DNA326667, 5425 | DNA326723, 5532 |
| DNA326668, 5428 | DNA326724, 5534 |
| DNA326669, 5430 | DNA326725, 5536 |
| DNA326670, 5432 | DNA326726, 5537 |
| DNA326671, 5436 | DNA326727, 5539 |
| DNA326672, 5438 | DNA326728, 5541 |
| DNA326673, 5439 | DNA326729, 5544 |
| DNA326674, 5440 | DNA326730, 5546 |
| DNA326675, 5443 | DNA326731, 5548 |
| DNA326676, 5444 | DNA326732, 5549 |
| DNA326677, 5445 | DNA326733, 5552 |
| DNA326678, 5446 | DNA326734, 5554 |
| DNA326679, 5447 | DNA326735, 5556 |
| DNA326680, 5450 | DNA326736, 5558 |
| DNA326681, 5451 | DNA326737, 5560 |
| DNA326682, 5453 | DNA326738, 5564 |
| DNA326683, 5454 | DNA326739, 5566 |
| DNA326684, 5456 | DNA326740, 5570 |
| DNA326685, 5458 | DNA326741, 5571 |
| DNA326686, 5460 | DNA326742, 5573 |
| DNA326687, 5461 | DNA326743, 5574 |
| DNA326688, 5462 | DNA326744, 5578 |
| DNA326689, 5463 | DNA326745, 5580 |
| DNA326690, 5465 | DNA326746, 5582 |
| DNA326691, 5466 | DNA326747, 5584 |
| DNA326692, 5468 | DNA326748, 5586 |
| DNA326693, 5470 | DNA326749, 5588 |
| DNA326694, 5472 | DNA326750, 5592 |
| DNA326751, 5595 | DNA326807, 5712 |
| DNA326752, 5597 | DNA326808, 5713 |
| DNA326753, 5598 | DNA326809, 5715 |
| DNA326754, 5600 | DNA326810, 5717 |
| DNA326755, 5602 | DNA326811, 5719 |
| DNA326756, 5603 | DNA326812, 5723 |
| DNA326757, 5605 | DNA326813, 5725 |
| DNA326758, 5607 | DNA326814, 5727 |
| DNA326759, 5608 | DNA326815, 5728 |
| DNA326760, 5610 | DNA326816, 5729 |
| DNA326761, 5612 | DNA326817, 5731 |
| DNA326762, 5613 | DNA326818, 5733 |
| DNA326763, 5617 | DNA326819, 5736 |
| DNA326764, 5619 | DNA326820, 5740 |
| DNA326765, 5621 | DNA326821, 5742 |
| DNA326766, 5623 | DNA326822, 5744 |
| DNA326767, 5629 | DNA326823, 5749 |
| DNA326768, 5631 | DNA326824, 5750 |
| DNA326769, 5633 | DNA326825, 5752 |
| DNA326770, 5635 | DNA326826, 5754 |
| DNA326771, 5636 | DNA326827, 5756 |
| DNA326772, 5642 | DNA326828, 5757 |
| DNA326773, 5644 | DNA326829, 5759 |
| DNA326774, 5646 | DNA326830, 5762 |
| DNA326775, 5647 | DNA326831, 5763 |
| DNA326776, 5648 | DNA326832, 5765 |
| DNA326777, 5650 | DNA326833, 5766 |
| DNA326778, 5652 | DNA326834, 5768 |
| DNA326779, 5656 | DNA326835, 5769 |
| DNA326780, 5658 | DNA326836, 5773 |
| DNA326781, 5660 | DNA326837, 5776 |
| DNA326782, 5661 | DNA326838, 5778 |
| DNA326783, 5663 | DNA326839, 5779 |
| DNA326784, 5665 | DNA326840, 5781 |
| DNA326785, 5667 | DNA326841, 5783 |
| DNA326786, 5670 | DNA326842, 5787 |
| DNA326787, 5671 | DNA326843, 5793 |
| DNA326788, 5673 | DNA326844, 5794 |
| DNA326789, 5674 | DNA326845, 5795 |
| DNA326790, 5675 | DNA326846, 5796 |
| DNA326791, 5678 | DNA326847, 5798 |
| DNA326792, 5683 | DNA326848, 5800 |
| DNA326793, 5687 | DNA326849, 5802 |
| DNA326794, 5688 | DNA326850, 5804 |
| DNA326795, 5689 | DNA326851, 5806 |
| DNA326796, 5691 | DNA326852, 5808 |
| DNA326797, 5693 | DNA326853, 5809 |
| DNA326798, 5695 | DNA326854, 5811 |
| DNA326799, 5696 | DNA326855, 5813 |
| DNA326800, 5698 | DNA326856, 5816 |
| DNA326801, 5700 | DNA326857, 5818 |
| DNA326802, 5701 | DNA326858, 5819 |
| DNA326803, 5705 | DNA326859, 5821 |
| DNA326804, 5706 | DNA326860, 5823 |
| DNA326805, 5708 | DNA326861, 5824 |
| DNA326806, 5710 | DNA326862, 5826 |
| DNA326863, 5828 | DNA326919, 5945 |
| DNA326864, 5832 | DNA326920, 5946 |
| DNA326865, 5834 | DNA326921, 5949 |
| DNA326866, 5838 | DNA326922, 5950 |
| DNA326867, 5840 | DNA326923, 5951 |
| DNA326868, 5842 | DNA326924, 5953 |
| DNA326869, 5846 | DNA326925, 5954 |
| DNA326870, 5847 | DNA326926, 5958 |
| DNA326871, 5849 | DNA326927, 5960 |
| DNA326872, 5851 | DNA326928, 5961 |
| DNA326873, 5853 | DNA326929, 5963 |
| DNA326874, 5855 | DNA326930, 5964 |
| DNA326875, 5857 | DNA326931, 5967 |
| DNA326876, 5859 | DNA326932, 5968 |
| DNA326877, 5861 | DNA326933, 5969 |
| DNA326878, 5863 | DNA326934, 5971 |
| DNA326879, 5865 | DNA326935, 5975 |
| DNA326880, 5867 | DNA326936, 5977 |
| DNA326881, 5869 | DNA326937, 5979 |
| DNA326882, 5871 | DNA326938, 5981 |
| DNA326883, 5875 | DNA326939, 5983 |
| DNA326884, 5876 | DNA326940, 5985 |
| DNA326885, 5877 | DNA326941, 5986 |
| DNA326886, 5878 | DNA326942, 5987 |
| DNA326887, 5879 | DNA326943, 5991 |
| DNA326888, 5883 | DNA326944, 5993 |
| DNA326889, 5887 | DNA326945, 5996 |
| DNA326890, 5889 | DNA326946, 5998 |
| DNA326891, 5894 | DNA326947, 5999 |
| DNA326892, 5898 | DNA326948, 6001 |
| DNA326893, 5900 | DNA326949, 6007 |
| DNA326894, 5902 | DNA326950, 6009 |
| DNA326895, 5903 | DNA326951, 6013 |
| DNA326896, 5905 | DNA326952, 6014 |
| DNA326897, 5907 | DNA326953, 6015 |
| DNA326898, 5908 | DNA326954, 6017 |
| DNA326899, 5910 | DNA326955, 6019 |
| DNA326900, 5911 | DNA326956, 6022 |
| DNA326901, 5913 | DNA326957, 6024 |
| DNA326902, 5914 | DNA326958, 6025 |
| DNA326903, 5915 | DNA326959, 6029 |
| DNA326904, 5917 | DNA326960, 6031 |
| DNA326905, 5919 | DNA326961, 6032 |
| DNA326906, 5923 | DNA326962, 6036 |
| DNA326907, 5924 | DNA326963, 6040 |
| DNA326908, 5925 | DNA326964, 6042 |
| DNA326909, 5926 | DNA326965, 6043 |
| DNA326910, 5927 | DNA326966, 6047 |
| DNA326911, 5928 | DNA326967, 6049 |
| DNA326912, 5929 | DNA326968, 6051 |
| DNA326913, 5930 | DNA326969, 6052 |
| DNA326914, 5931 | DNA326970, 6054 |
| DNA326915, 5933 | DNA326971, 6056 |
| DNA326916, 5937 | DNA326972, 6058 |
| DNA326917, 5941 | DNA326973, 6060 |
| DNA326918, 5943 | DNA326974, 6061 |
| DNA326975, 6063 | DNA327031, 6165 |
| DNA326976, 6064 | DNA327032, 6167 |
| DNA326977, 6065 | DNA327033, 6169 |
| DNA326978, 6066 | DNA327034, 6170 |
| DNA326979, 6070 | DNA327035, 6172 |
| DNA326980, 6072 | DNA327036, 6173 |
| DNA326981, 6074 | DNA327037, 6174 |
| DNA326982, 6077 | DNA327038, 6176 |
| DNA326983, 6081 | DNA327039, 6177 |
| DNA326984, 6083 | DNA327040, 6179 |
| DNA326985, 6085 | DNA327041, 6183 |
| DNA326986, 6087 | DNA327042, 6185 |
| DNA326987, 6088 | DNA327043, 6189 |
| DNA326988, 6089 | DNA327044, 6192 |
| DNA326989, 6090 | DNA327045, 6194 |
| DNA326990, 6091 | DNA327046, 6196 |
| DNA326991, 6093 | DNA327047, 6199 |
| DNA326992, 6094 | DNA327048, 6201 |
| DNA326993, 6095 | DNA327049, 6203 |
| DNA326994, 6097 | DNA327050, 6204 |
| DNA326995, 6099 | DNA327051, 6206 |
| DNA326996, 6103 | DNA327052, 6207 |
| DNA326997, 6106 | DNA327053, 6209 |
| DNA326998, 6108 | DNA327054, 6210 |
| DNA326999, 6109 | DNA327055, 6212 |
| DNA327000, 6111 | DNA327056, 6216 |
| DNA327001, 6113 | DNA327057, 6218 |
| DNA327002, 6114 | DNA327058, 6220 |
| DNA327003, 6116 | DNA327059, 6222 |
| DNA327004, 6118 | DNA327060, 6224 |
| DNA327005, 6119 | DNA327061, 6226 |
| DNA327006, 6121 | DNA327062, 6227 |
| DNA327007, 6122 | DNA327063, 6228 |
| DNA327008, 6123 | DNA327064, 6229 |
| DNA327009, 6124 | DNA327065, 6232 |
| DNA327010, 6128 | DNA327066, 6233 |
| DNA327011, 6130 | DNA327067, 6235 |
| DNA327012, 6131 | DNA327068, 6237 |
| DNA327013, 6132 | DNA327069, 6238 |
| DNA327014, 6134 | DNA327070, 6241 |
| DNA327015, 6136 | DNA327071, 6242 |
| DNA327016, 6138 | DNA327072, 6244 |
| DNA327017, 6140 | DNA327073, 6246 |
| DNA327018, 6142 | DNA327074, 6248 |
| DNA327019, 6143 | DNA327075, 6250 |
| DNA327020, 6145 | DNA327076, 6251 |
| DNA327021, 6146 | DNA327077, 6253 |
| DNA327022, 6151 | DNA327078, 6255 |
| DNA327023, 6152 | DNA327079, 6256 |
| DNA327024, 6153 | DNA327080, 6259 |
| DNA327025, 6155 | DNA327081, 6261 |
| DNA327026, 6157 | DNA327082, 6263 |
| DNA327027, 6158 | DNA327083, 6265 |
| DNA327028, 6159 | DNA327084, 6267 |
| DNA327029, 6161 | DNA327085, 6268 |
| DNA327030, 6163 | DNA327086, 6269 |
| DNA327087, 6274 | DNA88051, 898 |
| DNA327088, 6275 | DNA88084, 5511 |
| DNA327089, 6276 | DNA88100, 1089 |
| DNA327090, 6278 | DNA88114, 3452 |
| DNA327091, 6280 | DNA88176, 3333 |
| DNA327092, 6281 | DNA88239, 5791 |
| DNA327093, 6282 | DNA88261, 4579 |
| DNA327094, 6284 | DNA88281, 5050 |
| DNA327095, 6289 | DNA88350, 2796 |
| DNA327096, 6291 | DNA88378, 4845 |
| DNA327097, 6293 | DNA88430, 4963 |
| DNA327098, 6295 | DNA88457, 5040 |
| DNA327099, 6297 | DNA88547, 1223 |
| DNA327100, 6299 | DNA88554, 4903 |
| DNA327101, 6300 | DNA88562, 2961 |
| DNA327102, 6302 | DNA88569, 5789 |
| DNA327103, 6304 | DNA89239, 1327 |
| DNA327104, 6306 | DNA89242, 2695 |
| DNA327105, 6308 | DNA97285, 3175 |
| DNA327106, 6310 | DNA97290, 4887 |
| DNA327107, 6311 | DNA97293, 4421 |
| DNA327108, 6313 | DNA97298, 5734 |
| DNA327109, 6315 | DNA97300, 4687 |
| DNA327110, 6316 | |
| DNA327111, 6320 | |
| DNA327112, 6323 | |
| DNA327113, 6325 | |
| DNA327114, 6326 | |
| DNA327115, 6328 | |
| DNA327116, 6329 | |
| DNA327117, 6330 | |
| DNA327118, 6336 | |
| DNA327119, 6346 | |
| DNA327120, 6348 | |
| DNA327121, 6349 | |
| DNA327122, 6350 | |
| DNA327123, 6351 | |
| DNA327124, 6352 | |
| DNA327125, 6353 | |
| DNA327126, 6354 | |
| DNA327127, 6355 | |
| DNA66475, 4796 | |
| DNA75863, 3245 | |
| DNA76504, 6270 | |
| DNA79101, 3678 | |
| DNA79129, 1352 | |
| DNA79313, 3524 | |
| DNA82328, 624 | |
| DNA83020, 1671 | |
| DNA83022, 2495 | |
| DNA83046, 558 | |
| DNA83085, 173 | |
| DNA83141, 2361 | |
| DNA83154, 5590 | |
| DNA83170, 5679 | |
| DNA83180, 3476 | |

PRO Index (to Figure number)

PRO, 1189
PRO10002, 487
PRO10194, 2441
PRO10297, 1479
PRO10360, 1923
PRO10400, 4928
PRO10404, 3952
PRO10485, 5127
PRO10498, 967
PRO10602, 1207
PRO10685, 1633
PRO10692, 644
PRO10723, 6245
PRO10760, 211
PRO1077, 5094
PRO10824, 2652
PRO10838, 3657
PRO10849, 1709
PRO10935, 6279
PRO11048, 1285
PRO11077, 1571
PRO1108, 2532
PRO1112, 2003
PRO11139, 2981
PRO11197, 833
PRO11213, 3655
PRO11262, 3172
PRO11265, 2589
PRO11403, 902, 4970
PRO11582, 556
PRO11601, 3521
PRO11691, 3186
PRO1182, 646
PRO119, 2229
PRO11982, 3915
PRO1204, 4797
PRO12077, 1420
PRO12130, 5315
PRO12134, 6006
PRO12135, 5897
PRO12187, 3412
PRO12198, 4142
PRO12199, 682
PRO12224, 3205
PRO12265, 4937
PRO12324, 5704
PRO124, 3121
PRO12416, 1733
PRO12448, 3385
PRO12460, 5722
PRO12468, 2185
PRO1248, 565
PRO12490, 6055
PRO12520, 1025
PRO12565, 1146
PRO12573, 3527
PRO12618, 45
PRO12683, 4399
PRO12774, 4306
PRO12779, 1154
PRO12792, 807
PRO12797, 2035
PRO12800, 5503
PRO12806, 4954
PRO12813, 3014
PRO12822, 5429
PRO12838, 2547
PRO12839, 3758
PRO12841, 1067
PRO12845, 6023
PRO1285, 1665
PRO12851, 2905
PRO12878, 3250
PRO12886, 6021
PRO12892, 5477
PRO12902, 3467
PRO12916, 4080
PRO1314, 1239
PRO1555, 2457
PRO1707, 625
PRO1720, 5782
PRO1869, 3909
PRO188, 530
PRO1910, 2835
PRO1927, 1847
PRO19615, 1822
PRO19933, 2109
PRO201, 5209
PRO20117, 3257, 3259
PRO20136, 49
PRO2018, 3246
PRO2042, 2496
PRO2054, 4066
PRO2065, 5780
PRO2066, 4049
PRO2077, 1217
PRO2109, 5591
PRO2146, 899
PRO21481, 2669
PRO2172, 1090
PRO21728, 5837
PRO21773, 3666
PRO21887, 4783
PRO21924, 3481
PRO2198, 4639
PRO22196, 94
PRO22262, 168
PRO22304, 147
PRO224, 5217
PRO22481, 6028
PRO22613, 5284
PRO22637, 4569
PRO2267, 5051
PRO2269, 61
PRO22771, 1625
PRO22897, 2339
PRO22907, 2634, 2636
PRO231, 329
PRO23123, 999
PRO23124, 949, 951
PRO23201, 2615
PRO23231, 6059
PRO23238, 5589
PRO23248, 2568
PRO23300, 586
PRO23362, 2194
PRO23364, 2948
PRO2355, 4512
PRO2373, 6125
PRO23746, 13
PRO23794, 5251
PRO23797, 2024, 2151
PRO23845, 5394
PRO23942, 429
PRO24002, 5748
PRO24021, 6317
PRO24028, 855
PRO24075, 4531
PRO24077, 5761
PRO24091, 978
PRO2420, 5790
PRO24831, 3307
PRO24851, 577
PRO24856, 125
PRO25115, 4878
PRO25245, 6312
PRO25302, 5882
PRO2537, 6271
PRO2549, 3679
PRO2551, 1353
PRO2555, 3525
PRO2560, 5096
PRO2561, 1672
PRO2569, 559
PRO2570, 2477
PRO2583, 174
PRO25845, 3298
PRO25849, 1853
PRO25881, 5498
PRO25985, 3156
PRO2604, 2362
PRO2610, 981
PRO2615, 5680
PRO26194, 82
PRO2622, 3477
PRO26228, 983
PRO2644, 5512
PRO2660, 3453
PRO2665, 922
PRO2672, 4550
PRO2685, 3334
PRO2711, 5792
PRO2718, 5282
PRO2719, 4580
PRO2720, 4219
PRO2732, 4175
PRO2733, 2443
PRO2758, 2797
PRO2769, 4846
PRO2788, 4964
PRO2799, 5041
PRO283, 3664
PRO2837, 1224
PRO2839, 4904
PRO2841, 3741, 3743
PRO2842, 2962
PRO2846, 3661
PRO2851, 177
PRO28687, 5880
PRO287, 1277
PRO2871, 3995
PRO2875, 2974
PRO2906, 1328
PRO2907, 2696
PRO292, 3134
PRO29371, 5329
PRO302, 4918
PRO303, 4409
PRO329, 504
PRO3344, 2484
PRO33679, 368
PRO33717, 3963
PRO33818, 2773
PRO34043, 6205
PRO34073, 3052
PRO34151, 5479
PRO34323, 5259
PRO34473, 2783
PRO3449, 3601
PRO34531, 6076
PRO34544, 1676
PRO34557, 4183
PRO34584, 6186
PRO36020, 1741
PRO36047, 1490
PRO36055, 1331
PRO36058, 1735
PRO36093, 2768
PRO36094, 2175
PRO36095, 3474
PRO36112, 4043
PRO36118, 6339
PRO36134, 2507
PRO36184, 6215
PRO36215, 3377
PRO36263, 6335
PRO36272, 357
PRO3629, 4355, 4357
PRO36305, 1960
PRO36316, 1958
PRO3632, 3176
PRO36328, 3977
PRO3637, 4888
PRO36372, 1829
PRO36373, 1129
PRO36382, 1447
PRO36383, 1512
PRO36384, 1516
PRO3640, 4422
PRO36417, 5948
PRO3645, 5735
PRO36468, 554
PRO3647, 4688
PRO36474, 5518
PRO36477, 3730
PRO36491, 3490
PRO36543, 3207
PRO36568, 3993
PRO36588, 410
PRO36680, 3005
PRO36693, 2656
PRO36723, 272
PRO36725, 106
PRO36735, 1096
PRO36787, 4096
PRO36800, 2459
PRO36808, 2671
PRO36841, 1919
PRO36852, 4821
PRO36872, 5922
PRO36879, 2263
PRO36881, 1792
PRO36891, 742
PRO36959, 2566
PRO36963, 5490
PRO36970, 3456
PRO37010, 1109
PRO37012, 5976
PRO37023, 2394
PRO37024, 5957
PRO37073, 2987
PRO37080, 5936
PRO37082, 475
PRO37083, 6160
PRO37091, 1765
PRO37109, 4225
PRO37221, 5746
PRO37234, 3499
PRO37256, 437
PRO37316, 3867
PRO37335, 1690
PRO37476, 6333
PRO37518, 4940
PRO37534, 4890
PRO37535, 385
PRO37540, 4990
PRO37547, 4743
PRO37551, 3221
PRO37555, 3594
PRO37557, 3629
PRO37628, 685
PRO37634, 3725
PRO37635, 2965
PRO37636, 1574
PRO37644, 6273
PRO37653, 815
PRO37654, 3589
PRO37667, 1887
PRO37669, 4171
PRO37675, 924
PRO37676, 158
PRO37697, 4627
PRO37709, 551
PRO37712, 5353
PRO37730, 2513
PRO37731, 2243
PRO37743, 5233
PRO37764, 4934
PRO37770, 1166
PRO37783, 1813
PRO37784, 3985
PRO37791, 4793
PRO37806, 498
PRO37811, 5682
PRO37905, 1943
PRO37935, 5772
PRO37937, 3721
PRO37938, 2461
PRO37951, 5164
PRO37954, 2692
PRO37961, 4343
PRO37967, 595
PRO37972, 3077
PRO37991, 804
PRO37992, 347
PRO38008, 699
PRO38010, 3459
PRO38021, 6217
PRO38022, 4162
PRO38028, 2667
PRO38038, 1509
PRO38040, 375
PRO38066, 5810
PRO38070, 1962
PRO38101, 5565
PRO38119, 6286
PRO38152, 6148
PRO38227, 4892
PRO38258, 793
PRO38284, 37
PRO38311, 4359
PRO38336, 4842
PRO38380, 6322
PRO38387, 2100
PRO38392, 2207
PRO38406, 6198
PRO38464, 4336
PRO38480, 4373
PRO38496, 5133
PRO38730, 6343
PRO38852, 4215
PRO39030, 6105
PRO39127, 6182
PRO39201, 4983
PRO39530, 4643
PRO39648, 3009
PRO39773, 2399
PRO4, 2535
PRO41882, 2366
PRO42022, 6225
PRO42208, 4519
PRO4348, 3577
PRO4379, 4179
PRO4426, 3632
PRO44999, 579
PRO45014, 2183
PRO4544, 219
PRO4547, 650
PRO4569, 5577
PRO4583, 189
PRO4586, 6071
PRO4605, 1450
PRO4650, 5273
PRO4666, 3682
PRO4676, 1599
PRO4710, 1678
PRO4729, 4709
PRO47354, 5516
PRO4738, 4799
PRO4749, 2983
PRO4756, 5377
PRO4763, 458
PRO4789, 5995
PRO4793, 3848
PRO4798, 2071
PRO4801, 3314
PRO4813, 5845
PRO4814, 4582
PRO4832, 1150
PRO4833, 2991
PRO48357, 1082
PRO4836, 4111
PRO4841, 3479
PRO4852, 5775
PRO4870, 3312
PRO4872, 6082
PRO4873, 3684
PRO4884, 1950
PRO4885, 6345
PRO4900, 3050
PRO4904, 4064, 5495
PRO4908, 1780
PRO4912, 2275
PRO4914, 617
PRO4917, 712
PRO4918, 765
PRO49182, 4705
PRO49209, 1371
PRO49256, 6004
PRO49262, 1628
PRO49278, 6069
PRO49298, 3330
PRO49310, 4720
PRO49316, 995
PRO49352, 6046
PRO49409, 500
PRO49457, 604
PRO49639, 4488
PRO49642, 343
PRO49648, 2741
PRO49653, 5628
PRO49675, 5886
PRO49685, 1156
PRO49722, 1317
PRO49726, 3469
PRO4984, 1909
PRO49869, 2694
PRO49875, 3778
PRO49879, 4375
PRO49881, 6319
PRO49883, 6258
PRO49888, 4899
PRO49967, 4578
PRO50083, 5891
PRO50095, 1133
PRO50134, 5940
PRO50165, 3114
PRO50409, 4209
PRO50438, 4266
PRO50481, 4748
PRO50582, 1927
PRO50596, 860
PRO50658, 4613
PRO50756, 3110
PRO51109, 1273
PRO51119, 6102
PRO51121, 3512
PRO51389, 2055
PRO51539, 200
PRO51565, 5514
PRO51586, 5147
PRO51744, 5057
PRO51767, 5388
PRO51771, 5435
PRO51775, 4363
PRO51815, 4371
PRO51836, 546
PRO51851, 1643
PRO51901, 2747
PRO52010, 4855
PRO52083, 1438
PRO52101, 5507
PRO52119, 5966
PRO52449, 403
PRO52492, 3416
PRO52537, 2924
PRO54594, 4204
PRO57307, 3327
PRO57854, 3102
PRO57901, 5594
PRO57917, 4060
PRO57942, 5815
PRO58006, 1855
PRO58042, 5313
PRO58046, 5123
PRO58092, 3899
PRO58118, 268
PRO58140, 1196
PRO58155, 5874
PRO58177, 1257
PRO58198, 6127
PRO58207, 3285
PRO58213, 1688
PRO58219, 1647
PRO58232, 5990
PRO58259, 1271
PRO58263, 2553
PRO58292, 5299
PRO58308, 1063
PRO58328, 1098
PRO58348, 3094
PRO58410, 3865
PRO58437, 2370
PRO58440, 3688
PRO58446, 5268
PRO58523, 3209
PRO58543, 747
PRO58606, 3300
PRO58642, 3897
PRO58702, 5207
PRO58784, 1100
PRO58837, 3592
PRO58875, 6240
PRO58939, 2877
PRO58974, 471
PRO58984, 1893
PRO58986, 1387
PRO58991, 5235
PRO58993, 1804
PRO59001, 3817
PRO59022, 6243
PRO59040, 1851
PRO59042, 3824
PRO59043, 3056
PRO59061, 6012
PRO59074, 2372
PRO59084, 3296
PRO59099, 128
PRO59136, 795
PRO59142, 5649, 5651
PRO59168, 6154
PRO59220, 5361
PRO59230, 2551
PRO59262, 3440
PRO59264, 5505
PRO59285, 6080
PRO59305, 4844
PRO59309, 4806
PRO59313, 959
PRO59321, 2663
PRO59328, 4912
PRO59332, 289
PRO59339, 5677
PRO59351, 2856
PRO59365, 1998
PRO59380, 2397
PRO59384, 752
PRO59441, 6139
PRO59491, 4508
PRO59504, 1094
PRO59544, 762
PRO59546, 230
PRO59558, 704
PRO59579, 2674
PRO59629, 3381
PRO59647, 3551
PRO59669, 5904
PRO59717, 2105
PRO59721, 3272
PRO59725, 462
PRO59785, 3728
PRO59868, 6188
PRO59895, 935
PRO59913, 1722
PRO60006, 2752
PRO60008, 3282
PRO60070, 4102
PRO60115, 2807
PRO60121, 5053
PRO60123, 3393
PRO60127, 2661
PRO6018, 395
PRO60207, 1698
PRO60261, 520
PRO60298, 203
PRO60311, 3345
PRO60321, 2601
PRO60325, 4450
PRO60333, 5626
PRO60360, 2349
PRO60397, 882
PRO60438, 1867
PRO60475, 2735
PRO60499, 4262
PRO60542, 1397
PRO60575, 4087
PRO60579, 2181
PRO60603, 5427
PRO60634, 3556
PRO60666, 3391
PRO60674, 5202
PRO60741, 1336
PRO60753, 872
PRO60781, 2715
PRO60800, 5073
PRO60815, 84
PRO60847, 3216
PRO60860, 236
PRO60924, 3575
PRO60945, 5743
PRO60956, 1495
PRO60979, 4813
PRO60991, 3087
PRO61085, 4268
PRO61113, 5070
PRO61125, 195
PRO61129, 5569
PRO61146, 397
PRO61219, 4260
PRO61238, 3323
PRO61246, 5003
PRO61250, 90
PRO61271, 6231
PRO61308, 5575
PRO61325, 781
PRO61327, 5786
PRO61349, 5616
PRO61458, 5422
PRO61470, 6288
PRO61498, 5739
PRO61502, 3067
PRO61575, 5449
PRO61638, 349
PRO61661, 5024
PRO61679, 43
PRO61688, 2250
PRO61721, 2900
PRO61744, 6141
PRO61761, 689
PRO61799, 4361
PRO61812, 2237
PRO61824, 2247
PRO61870, 1183
PRO61897, 2795
PRO61938, 6191
PRO61948, 4477
PRO61977, 5278
PRO61999, 3913
PRO62039, 5116
PRO62065, 5893
PRO62069, 2865
PRO62075, 5442
PRO62077, 516
PRO62099, 4070
PRO62108, 2492
PRO62110, 4517
PRO62112, 5242
PRO62135, 1831
PRO62153, 1171
PRO62212, 5524
PRO62225, 5179
PRO62236, 2781
PRO62239, 750
PRO62244, 2177
PRO62273, 3764
PRO62302, 5162
PRO62328, 6000
PRO62389, 181
PRO62466, 6327
PRO62500, 5407
PRO62518, 193
PRO62529, 341
PRO62531, 5200
PRO62574, 453
PRO62582, 5543
PRO62588, 6150
PRO62607, 637
PRO62617, 2882
PRO62760, 931
PRO62770, 663
PRO62780, 4666
PRO62786, 745
PRO62849, 293
PRO62852, 4301
PRO62882, 4321
PRO62893, 652
PRO62899, 5103
PRO62927, 865
PRO62981, 4717
PRO63000, 2724
PRO63009, 2233
PRO63052, 1972
PRO63068, 4565
PRO63082, 1680
PRO63226, 5238
PRO63253, 1905
PRO63299, 6341
PRO6360, 1077
PRO6373, 2213
PRO65, 848
PRO66265, 4670
PRO66275, 3901
PRO66279, 2127
PRO66282, 2129
PRO69461, 3302
PRO69463, 32
PRO69471, 3840
PRO69473, 3027, 3760
PRO69475, 2266
PRO69486, 5906
PRO69496, 2702
PRO69506, 1953
PRO69513, 5015
PRO69518, 5280
PRO69521, 1901
PRO69523, 3011
PRO69528, 3237
PRO69531, 906
PRO69533, 5669
PRO69541, 5655
PRO69542, 2764
PRO69549, 3461
PRO69554, 1583
PRO69560, 5686
PRO69561, 4920
PRO69568, 3254
PRO69584, 1970
PRO69595, 4243
PRO69617, 4521
PRO69635, 3138
PRO69674, 3219
PRO69681, 1301
PRO69682, 4901
PRO69684, 4779
PRO70011, 2704
PRO70138, 1968
PRO70258, 656
PRO70276, 4447
PRO70290, 2762
PRO703, 380
PRO70327, 1637, 1639
PRO70331, 6030
PRO70333, 541
PRO70383, 6035
PRO70385, 5551
PRO70393, 2008
PRO70433, 351
PRO70449, 4729
PRO70453, 3621
PRO70536, 1460
PRO70544, 2033
PRO70595, 2681
PRO70675, 991
PRO70694, 3786
PRO70703, 4976
PRO70754, 439
PRO70810, 5639
PRO70812, 4700
PRO70989, 3828, 3830
PRO70993, 1719
PRO71031, 3433, 3435
PRO71057, 2997
PRO71085, 5563
PRO71088, 1947
PRO71089, 5641
PRO71091, 2591
PRO71093, 370
PRO71095, 1964
PRO71096, 3888
PRO71097, 5831
PRO71103, 1148
PRO71106, 1875
PRO71111, 2436
PRO71112, 221
PRO71120, 3718
PRO71125, 1985, 1987
PRO71130, 4576
PRO71133, 2255
PRO71136, 2309
PRO71141, 4715
PRO71142, 1913
PRO71145, 6039
PRO71146, 372
PRO71211, 2343
PRO71242, 5974
PRO7143, 4986
PRO730, 4274
PRO7427, 2239
PRO7445, 2594
PRO80480, 5
PRO80481, 7
PRO80482, 9
PRO80483, 11
PRO80484, 16
PRO80485, 18
PRO80487, 21
PRO80488, 23
PRO80489, 25
PRO80490, 27
PRO80492, 30
PRO80493, 35
PRO80494, 39
PRO80497, 47
PRO80498, 51
PRO80499, 53
PRO80501, 56
PRO80505, 63
PRO80506, 65
PRO80510, 70
PRO80511, 72
PRO80512, 74
PRO80517, 80
PRO80518, 86
PRO80519, 88
PRO80520, 92
PRO80521, 96
PRO80524, 100
PRO80527, 104
PRO80528, 108
PRO80530, 111
PRO80533, 115
PRO80534, 117
PRO80535, 119
PRO80536, 121
PRO80537, 123
PRO80542, 132
PRO80547, 138
PRO80550, 143
PRO80553, 149
PRO80554, 151
PRO80555, 153
PRO80557, 156
PRO80558, 160
PRO80559, 162
PRO80560, 164
PRO80561, 166
PRO80562, 170
PRO80563, 172
PRO80565, 179
PRO80567, 184
PRO80568, 186
PRO80570, 191
PRO80571, 197
PRO80574, 205
PRO80575, 207
PRO80576, 209
PRO80579, 215
PRO80580, 217
PRO80581, 223
PRO80582, 225
PRO80583, 227
PRO80584, 232
PRO80585, 234
PRO80586, 238
PRO80587, 240
PRO80588, 242
PRO80591, 246
PRO80592, 248
PRO80593, 252
PRO80595, 255
PRO80597, 258
PRO80599, 261
PRO80600, 263
PRO80602, 266
PRO80603, 270
PRO80604, 274
PRO80607, 278
PRO80611, 283
PRO80612, 285
PRO80613, 287
PRO80614, 291
PRO80617, 297
PRO80618, 299
PRO80619, 301
PRO80620, 303
PRO80621, 305
PRO80622, 307
PRO80623, 309
PRO80624, 311
PRO80625, 313
PRO80627, 316
PRO80630, 320
PRO80631, 322
PRO80633, 325
PRO80638, 333
PRO80639, 335
PRO80640, 337
PRO80641, 339
PRO80642, 345
PRO80644, 355
PRO80645, 359
PRO80646, 362
PRO80648, 365
PRO80651, 378
PRO80652, 382
PRO80654, 387
PRO80656, 390
PRO80657, 393
PRO80658, 399
PRO80659, 401
PRO80660, 405
PRO80661, 407
PRO80664, 413
PRO80665, 415
PRO80666, 417
PRO80667, 419
PRO80668, 421
PRO80669, 423
PRO80670, 425
PRO80671, 427
PRO80672, 431
PRO80675, 435
PRO80676, 441
PRO80677, 443
PRO80678, 445
PRO80679, 447
PRO80680, 449
PRO80684, 456
PRO80685, 460
PRO80686, 464
PRO80688, 467
PRO80689, 469
PRO80693, 478
PRO80694, 480
PRO80695, 482
PRO80699, 489
PRO80700, 491
PRO80704, 496
PRO80705, 502
PRO80706, 506
PRO80707, 508
PRO80708, 510
PRO80709, 512
PRO80710, 514
PRO80711, 518
PRO80714, 526
PRO80715, 528
PRO80717, 533
PRO80719, 536
PRO80720, 539
PRO80725, 549
PRO80730, 563
PRO80734, 570
PRO80735, 572
PRO80736, 574
PRO80740, 584
PRO80741, 588
PRO80742, 590
PRO80745, 597
PRO80752, 607
PRO80753, 609
PRO80754, 611
PRO80755, 613
PRO80756, 615
PRO80759, 621
PRO80760, 623
PRO80761, 627
PRO80762, 629
PRO80763, 631
PRO80764, 633
PRO80765, 635
PRO80767, 640
PRO80770, 648
PRO80771, 654
PRO80772, 658
PRO80773, 660
PRO80775, 666
PRO80776, 668
PRO80778, 671
PRO80779, 673
PRO80780, 675
PRO80781, 677
PRO80782, 679
PRO80785, 687
PRO80786, 691
PRO80787, 693
PRO80788, 695
PRO80789, 697
PRO80790, 702
PRO80791, 708
PRO80792, 710
PRO80793, 714
PRO80796, 718
PRO80797, 720
PRO80798, 722
PRO80799, 724
PRO80802, 728
PRO80803, 730
PRO80804, 732
PRO80806, 735
PRO80807, 737
PRO80808, 739
PRO80811, 754
PRO80812, 756
PRO80813, 758
PRO80814, 760
PRO80816, 767
PRO80817, 769
PRO80820, 774
PRO80823, 778
PRO80827, 786
PRO80830, 790
PRO80832, 797
PRO80833, 799
PRO80835, 802
PRO80837, 809
PRO80838, 811
PRO80839, 813
PRO80842, 819
PRO80843, 821
PRO80846, 826
PRO80847, 828
PRO80848, 830
PRO80850, 835
PRO80851, 837
PRO80853, 840
PRO80856, 844
PRO80857, 846
PRO80860, 852
PRO80866, 863
PRO80868, 868
PRO80869, 870
PRO80870, 874
PRO80871, 876
PRO80873, 879
PRO80875, 884
PRO80876, 886
PRO80877, 888
PRO80878, 890
PRO80879, 892
PRO80881, 895
PRO80882, 897
PRO80883, 904
PRO80884, 908
PRO80888, 913
PRO80889, 915
PRO80890, 917
PRO80891, 919
PRO80900, 937
PRO80901, 939
PRO80903, 943
PRO80904, 945
PRO80905, 947
PRO80906, 953
PRO80908, 956
PRO80910, 961
PRO80911, 963
PRO80915, 972
PRO80916, 974
PRO80917, 976
PRO80920, 987
PRO80921, 989
PRO80924, 997
PRO80925, 1001
PRO80926, 1003
PRO80927, 1005
PRO80929, 1008
PRO80930, 1010
PRO80932, 1013
PRO80933, 1015
PRO80934, 1017
PRO80935, 1019
PRO80936, 1021
PRO80937, 1023
PRO80938, 1027
PRO80941, 1031
PRO80942, 1033
PRO80943, 1035
PRO80945, 1038
PRO80949, 1044
PRO80950, 1046
PRO80951, 1048
PRO80952, 1050
PRO80953, 1052
PRO80954, 1054
PRO80955, 1056
PRO80956, 1058
PRO80958, 1061
PRO80959, 1065
PRO80960, 1069
PRO80961, 1071
PRO80962, 1073
PRO80966, 1080
PRO80967, 1084
PRO80969, 1087
PRO80970, 1092
PRO80971, 1102
PRO80972, 1104
PRO80974, 1107
PRO80975, 1111
PRO80977, 1114
PRO80978, 1116
PRO80979, 1118
PRO80983, 1124
PRO80984, 1126
PRO80988, 1135
PRO80990, 1138
PRO80993, 1142
PRO80994, 1144
PRO80995, 1152
PRO80996, 1158
PRO80997, 1160
PRO80999, 1164
PRO81000, 1168
PRO81002, 1173
PRO81003, 1175
PRO81004, 1177
PRO81005, 1179
PRO81006, 1181
PRO81007, 1185
PRO81010, 1191
PRO81012, 1194
PRO81015, 1200
PRO81022, 1210
PRO81023, 1212
PRO81025, 1215
PRO81026, 1219
PRO81028, 1222
PRO81029, 1226
PRO81030, 1228
PRO81031, 1230
PRO81033, 1233
PRO81034, 1236
PRO81036, 1241
PRO81040, 1247
PRO81041, 1249
PRO81042, 1251
PRO81043, 1253
PRO81046, 1259
PRO81047, 1261
PRO81053, 1269
PRO81056, 1279
PRO81057, 1281
PRO81058, 1283
PRO81059, 1287
PRO81064, 1293
PRO81068, 1298
PRO81070, 1303
PRO81071, 1305
PRO81072, 1307
PRO81073, 1309
PRO81074, 1311
PRO81079, 1319
PRO81080, 1321
PRO81082, 1324
PRO81083, 1326
PRO81086, 1334
PRO81088, 1339
PRO81090, 1342
PRO81093, 1346
PRO81095, 1349
PRO81096, 1351
PRO81097, 1355
PRO81098, 1357
PRO81102, 1362
PRO81106, 1367
PRO81107, 1373
PRO81108, 1375
PRO81109, 1377
PRO81110, 1379
PRO81111, 1381
PRO81112, 1383
PRO81113, 1385
PRO81114, 1389
PRO81115, 1391
PRO81116, 1393
PRO81117, 1395
PRO81118, 1399
PRO81119, 1401
PRO81120, 1403
PRO81121, 1405
PRO81122, 1407
PRO81123, 1409
PRO81124, 1411
PRO81127, 1415
PRO81131, 1422
PRO81134, 1426
PRO81135, 1428
PRO81137, 1431
PRO81141, 1436
PRO81146, 1444
PRO81148, 1454
PRO81150, 1458
PRO81151, 1462
PRO81156, 1467
PRO81163, 1475
PRO81166, 1481
PRO81169, 1486
PRO81170, 1488
PRO81171, 1492
PRO81174, 1498
PRO81176, 1501
PRO81177, 1503
PRO81178, 1505
PRO81179, 1507
PRO81181, 1514
PRO81182, 1518
PRO81184, 1521
PRO81185, 1523
PRO81188, 1527
PRO81189, 1529
PRO81195, 1536
PRO81196, 1538
PRO81199, 1542
PRO81202, 1546
PRO81213, 1558
PRO81214, 1560
PRO81216, 1563
PRO81219, 1566
PRO81221, 1569
PRO81223, 1576
PRO81224, 1578
PRO81225, 1580
PRO81227, 1585
PRO81230, 1589
PRO81234, 1594
PRO81238, 1601
PRO81239, 1603
PRO81240, 1605
PRO81241, 1607
PRO81246, 1613
PRO81248, 1616
PRO81249, 1618
PRO81250, 1620
PRO81254, 1630
PRO81256, 1635
PRO81259, 1645
PRO81260, 1649
PRO81261, 1651
PRO81262, 1653
PRO81264, 1657
PRO81265, 1659
PRO81266, 1661
PRO81269, 1667
PRO81272, 1674
PRO81273, 1682
PRO81274, 1684
PRO81275, 1686
PRO81276, 1692
PRO81277, 1694
PRO81278, 1696
PRO81280, 1703
PRO81281, 1705
PRO81282, 1707
PRO81286, 1715
PRO81287, 1717
PRO81289, 1725
PRO81290, 1727
PRO81291, 1729
PRO81292, 1731
PRO81293, 1737
PRO81294, 1739
PRO81295, 1743
PRO81296, 1745
PRO81297, 1747
PRO81298, 1749
PRO81299, 1751
PRO81300, 1753
PRO81301, 1755
PRO81302, 1757
PRO81303, 1759
PRO81304, 1761
PRO81305, 1763
PRO81306, 1767
PRO81307, 1769
PRO81308, 1771
PRO81309, 1773
PRO81310, 1775
PRO81311, 1777
PRO81313, 1782
PRO81314, 1784
PRO81315, 1786
PRO81316, 1788
PRO81317, 1790
PRO81318, 1794
PRO81319, 1796
PRO81323, 1801
PRO81325, 1806
PRO81327, 1809
PRO81330, 1815
PRO81336, 1825
PRO81337, 1827
PRO81338, 1833
PRO81339, 1835
PRO81340, 1837
PRO81341, 1839
PRO81345, 1844
PRO81347, 1849
PRO81354, 1864
PRO81355, 1869
PRO81356, 1873
PRO81359, 1881
PRO81362, 1885
PRO81363, 1889
PRO81364, 1891
PRO81365, 1895
PRO81366, 1897
PRO81367, 1899
PRO81368, 1903
PRO81369, 1907
PRO81370, 1911
PRO81371, 1915
PRO81372, 1917
PRO81375, 1925
PRO81377, 1930
PRO81379, 1933
PRO81380, 1935
PRO81387, 1945
PRO81394, 1966
PRO81395, 1974
PRO81396, 1976
PRO81397, 1978
PRO81398, 1980
PRO81399, 1982
PRO81400, 1989
PRO81401, 1991
PRO81402, 1993
PRO81404, 1996
PRO81406, 2001
PRO81407, 2005
PRO81409, 2010
PRO81410, 2012
PRO81411, 2014
PRO81414, 2018
PRO81415, 2020
PRO81416, 2022
PRO81417, 2026
PRO81418, 2028
PRO81419, 2030
PRO81421, 2038
PRO81422, 2040
PRO81424, 2043
PRO81426, 2046
PRO81427, 2048
PRO81429, 2051
PRO81430, 2053
PRO81435, 2061
PRO81436, 2063
PRO81439, 2067
PRO81441, 2073
PRO81443, 2076
PRO81444, 2078
PRO81446, 2082
PRO81447, 2084
PRO81448, 2086
PRO81449, 2088
PRO81453, 2093
PRO81454, 2095
PRO81455, 2097
PRO81457, 2102
PRO81462, 2112
PRO81464, 2115
PRO81465, 2117
PRO81467, 2120
PRO81471, 2125
PRO81474, 2133
PRO81476, 2136
PRO81477, 2138
PRO81481, 2143
PRO81486, 2149
PRO81487, 2153
PRO81490, 2156
PRO81491, 2158
PRO81494, 2162
PRO81495, 2164
PRO81496, 2166
PRO81498, 2169
PRO81499, 2171
PRO81500, 2173
PRO81501, 2179
PRO81502, 2187
PRO81503, 2189
PRO81505, 2192
PRO81506, 2196
PRO81508, 2199
PRO81509, 2201
PRO81514, 2209
PRO81515, 2211
PRO81516, 2215
PRO81517, 2217
PRO81520, 2221
PRO81521, 2223
PRO81523, 2227
PRO81524, 2231
PRO81525, 2235
PRO81526, 2241
PRO81529, 2252
PRO81531, 2257
PRO81534, 2261
PRO81536, 2268
PRO81537, 2270
PRO81538, 2272
PRO81540, 2277
PRO81541, 2279
PRO81544, 2283
PRO81545, 2285
PRO81546, 2287
PRO81547, 2289
PRO81548, 2291
PRO81550, 2294
PRO81551, 2296
PRO81552, 2298
PRO81556, 2303
PRO81557, 2305
PRO81558, 2307
PRO81561, 2314
PRO81566, 2320
PRO81569, 2324
PRO81571, 2327
PRO81572, 2330
PRO81573, 2332
PRO81574, 2334
PRO81575, 2336
PRO81578, 2341
PRO81579, 2345
PRO81580, 2347
PRO81584, 2354
PRO81585, 2356
PRO81586, 2358
PRO81587, 2360
PRO81588, 2364
PRO81589, 2368
PRO81590, 2374
PRO81591, 2377
PRO81592, 2379
PRO81594, 2382
PRO81595, 2384
PRO81598, 2388
PRO81599, 2390
PRO81600, 2392
PRO81602, 2401
PRO81605, 2405
PRO81607, 2408
PRO81608, 2410
PRO81609, 2412
PRO81611, 2415
PRO81614, 2419
PRO81615, 2421
PRO81617, 2424
PRO81618, 2426
PRO81619, 2428
PRO81621, 2431
PRO81625, 2438
PRO81627, 2445
PRO81629, 2448
PRO81630, 2450
PRO81631, 2452
PRO81634, 2463
PRO81635, 2465
PRO81637, 2468
PRO81641, 2474
PRO81643, 2481
PRO81645, 2486
PRO81647, 2489
PRO81649, 2494
PRO81650, 2498
PRO81651, 2500
PRO81652, 2502
PRO81653, 2504
PRO81654, 2509
PRO81655, 2511
PRO81657, 2516
PRO81658, 2518
PRO81659, 2520
PRO81660, 2522
PRO81661, 2524
PRO81664, 2528
PRO81668, 2537
PRO81669, 2539
PRO81671, 2542
PRO81673, 2545
PRO81674, 2549
PRO81675, 2555
PRO81685, 2570
PRO81687, 2573
PRO81689, 2576
PRO81690, 2578
PRO81691, 2580
PRO81695, 2585
PRO81696, 2587
PRO81699, 2597
PRO81700, 2599
PRO81701, 2603
PRO81704, 2607
PRO81705, 2610
PRO81708, 2617
PRO81711, 2621
PRO81714, 2624
PRO81719, 2630
PRO81720, 2632
PRO81722, 2639
PRO81725, 2643
PRO81729, 2648
PRO81730, 2650
PRO81731, 2654
PRO81732, 2658
PRO81734, 2665
PRO81736, 2678
PRO81738, 2683
PRO81739, 2685
PRO81740, 2687
PRO81742, 2690
PRO81743, 2698
PRO81746, 2706
PRO81748, 2709
PRO81751, 2713
PRO81752, 2717
PRO81753, 2719
PRO81754, 2721
PRO81759, 2729
PRO81760, 2731
PRO81761, 2733
PRO81762, 2737
PRO81763, 2739
PRO81764, 2743
PRO81765, 2745
PRO81766, 2749
PRO81768, 2754
PRO81771, 2758
PRO81775, 2770
PRO81778, 2776
PRO81780, 2779
PRO81781, 2785
PRO81783, 2788
PRO81785, 2791
PRO81786, 2793
PRO81792, 2805
PRO81794, 2810
PRO81795, 2812
PRO81796, 2814
PRO81797, 2816
PRO81800, 2821
PRO81801, 2823
PRO81804, 2827
PRO81805, 2829
PRO81806, 2831
PRO81809, 2837
PRO81811, 2840
PRO81812, 2842
PRO81813, 2844
PRO81815, 2848
PRO81816, 2850
PRO81817, 2853
PRO81819, 2858
PRO81821, 2861
PRO81822, 2863
PRO81823, 2867
PRO81824, 2869
PRO81826, 2872
PRO81831, 2880
PRO81832, 2884
PRO81833, 2886
PRO81834, 2888
PRO81835, 2890
PRO81836, 2892
PRO81837, 2894
PRO81838, 2896
PRO81841, 2903
PRO81842, 2907
PRO81846, 2912
PRO81848, 2915
PRO81849, 2917
PRO81851, 2920
PRO81855, 2928
PRO81858, 2932
PRO81861, 2936
PRO81862, 2938
PRO81863, 2940
PRO81865, 2943
PRO81867, 2946
PRO81868, 2950
PRO81869, 2954
PRO81870, 2956
PRO81871, 2958
PRO81872, 2960
PRO81874, 2967
PRO81875, 2969
PRO81877, 2972
PRO81881, 2979
PRO81882, 2985
PRO81883, 2989
PRO81884, 2993
PRO81885, 2995
PRO81887, 3001
PRO81888, 3003
PRO81889, 3007
PRO81893, 3018
PRO81895, 3021
PRO81896, 3023
PRO81897, 3025
PRO81899, 3030
PRO81900, 3032
PRO81901, 3034
PRO81902, 3036
PRO81903, 3038
PRO81904, 3040
PRO81905, 3043
PRO81907, 3046
PRO81908, 3048
PRO81909, 3054
PRO81912, 3060
PRO81913, 3062
PRO81914, 3064
PRO81916, 3069
PRO81917, 3071
PRO81922, 3079
PRO81923, 3081
PRO81924, 3083
PRO81925, 3085
PRO81926, 3090
PRO81927, 3092
PRO81928, 3096
PRO81929, 3098
PRO81930, 3100
PRO81932, 3105
PRO81934, 3108
PRO81935, 3112
PRO81936, 3116
PRO81937, 3118
PRO81939, 3123
PRO81941, 3126
PRO81942, 3128
PRO81943, 3130
PRO81944, 3132
PRO81945, 3140
PRO81946, 3143
PRO81947, 3145
PRO81948, 3147
PRO81950, 3150
PRO81953, 3154
PRO81954, 3158
PRO81955, 3160
PRO81956, 3162
PRO81957, 3164
PRO81958, 3166
PRO81959, 3168
PRO81962, 3174
PRO81964, 3179
PRO81965, 3181
PRO81970, 3189
PRO81971, 3191
PRO81977, 3198
PRO81978, 3200
PRO81980, 3203
PRO81981, 3211
PRO81982, 3213
PRO81988, 3226
PRO81990, 3229
PRO81991, 3231
PRO81992, 3233
PRO81993, 3235
PRO81994, 3239
PRO81995, 3241
PRO81996, 3243
PRO81999, 3252
PRO82002, 3262
PRO82004, 3265
PRO82005, 3267
PRO82009, 3274
PRO82011, 3277
PRO82013, 3280
PRO82018, 3290
PRO82019, 3292
PRO82023, 3305
PRO82024, 3309
PRO82027, 3317
PRO82028, 3319
PRO82029, 3321
PRO82032, 3332
PRO82034, 3338
PRO82035, 3340
PRO82037, 3343
PRO82038, 3347
PRO82040, 3351
PRO82042, 3354
PRO82043, 3356
PRO82045, 3360
PRO82046, 3362
PRO82050, 3368
PRO82051, 3370
PRO82052, 3372
PRO82054, 3375
PRO82055, 3379
PRO82056, 3383
PRO82057, 3387
PRO82058, 3389
PRO82060, 3396
PRO82064, 3402
PRO82066, 3405
PRO82068, 3408
PRO82069, 3410
PRO82072, 3419
PRO82073, 3421
PRO82074, 3423
PRO82075, 3425
PRO82078, 3431
PRO82080, 3438
PRO82082, 3443
PRO82083, 3445
PRO82084, 3447
PRO82085, 3449
PRO82091, 3464
PRO82093, 3471
PRO82097, 3484
PRO82099, 3487
PRO82101, 3492
PRO82104, 3497
PRO82106, 3502
PRO82107, 3505
PRO82109, 3508
PRO82110, 3510
PRO82111, 3514
PRO82112, 3516
PRO82113, 3518
PRO82115, 3523
PRO82117, 3530
PRO82120, 3534
PRO82122, 3537
PRO82125, 3541
PRO82127, 3544
PRO82129, 3547
PRO82130, 3549
PRO82131, 3553
PRO82133, 3558
PRO82137, 3563
PRO82138, 3565
PRO82139, 3567
PRO82140, 3569
PRO82143, 3573
PRO82152, 3587
PRO82155, 3597
PRO82158, 3603
PRO82159, 3605
PRO82160, 3607
PRO82161, 3609
PRO82162, 3611
PRO82163, 3613
PRO82164, 3615
PRO82165, 3617
PRO82166, 3619
PRO82167, 3623
PRO82168, 3625
PRO82169, 3627
PRO82174, 3637
PRO82175, 3639
PRO82179, 3644
PRO82181, 3647
PRO82182, 3649
PRO82183, 3651
PRO82184, 3653
PRO82188, 3670
PRO82190, 3673
PRO82191, 3675
PRO82192, 3677
PRO82194, 3686
PRO82195, 3690
PRO82196, 3692
PRO82197, 3694
PRO82198, 3696
PRO82199, 3698
PRO82201, 3701
PRO82202, 3703
PRO82204, 3706
PRO82206, 3709
PRO82207, 3711
PRO82208, 3713
PRO82210, 3716
PRO82212, 3723
PRO82213, 3733
PRO82214, 3735
PRO82215, 3738
PRO82218, 3745
PRO82219, 3747
PRO82220, 3749
PRO82221, 3751
PRO82223, 3756
PRO82224, 3762
PRO82227, 3768
PRO82228, 3770
PRO82232, 3776
PRO82233, 3780
PRO82234, 3782
PRO82235, 3784
PRO82237, 3789
PRO82238, 3791
PRO82239, 3793
PRO82240, 3795
PRO82243, 3799
PRO82244, 3801
PRO82245, 3803
PRO82247, 3806
PRO82248, 3808
PRO82250, 3811
PRO82252, 3813
PRO82253, 3815
PRO82255, 3820
PRO82256, 3822
PRO82259, 3832
PRO82263, 3838
PRO82264, 3842
PRO82265, 3844
PRO82266, 3846
PRO82267, 3850
PRO82268, 3852
PRO82269, 3854
PRO82272, 3858
PRO82273, 3860
PRO82275, 3863
PRO82278, 3871
PRO82279, 3873
PRO82280, 3875
PRO82283, 3879
PRO82285, 3882
PRO82287, 3885
PRO82289, 3890
PRO82290, 3892
PRO82291, 3894
PRO82295, 3905
PRO82296, 3907
PRO82297, 3911
PRO82300, 3919
PRO82302, 3922
PRO82305, 3927
PRO82306, 3929
PRO82311, 3934
PRO82312, 3936
PRO82314, 3939
PRO82315, 3941
PRO82316, 3943
PRO82317, 3945
PRO82318, 3947
PRO82321, 3954
PRO82322, 3956
PRO82325, 3961
PRO82326, 3965
PRO82329, 3970
PRO82330, 3972
PRO82331, 3974
PRO82333, 3979
PRO82334, 3982
PRO82338, 3989
PRO82342, 3998
PRO82343, 4000
PRO82344, 4002
PRO82345, 4004
PRO82347, 4007
PRO82348, 4009
PRO82349, 4011
PRO82350, 4013
PRO82351, 4015
PRO82352, 4017
PRO82355, 4021
PRO82356, 4023
PRO82357, 4026
PRO82358, 4028
PRO82359, 4030
PRO82364, 4036
PRO82365, 4038
PRO82367, 4041
PRO82369, 4047
PRO82370, 4051
PRO82371, 4053
PRO82373, 4056
PRO82374, 4058
PRO82375, 4062
PRO82381, 4076
PRO82382, 4078
PRO82383, 4083
PRO82384, 4085
PRO82385, 4089
PRO82388, 4093
PRO82391, 4099
PRO82393, 4104
PRO82395, 4107
PRO82396, 4109
PRO82397, 4113
PRO82400, 4117
PRO82408, 4126
PRO82409, 4128
PRO82411, 4131
PRO82415, 4137
PRO82417, 4140
PRO82418, 4144
PRO82419, 4146
PRO82421, 4149
PRO82422, 4151
PRO82423, 4153
PRO82424, 4155
PRO82425, 4158
PRO82428, 4164
PRO82429, 4166
PRO82431, 4169
PRO82432, 4173
PRO82433, 4177
PRO82434, 4181
PRO82435, 4185
PRO82437, 4188
PRO82438, 4190
PRO82439, 4192
PRO82440, 4194
PRO82441, 4196
PRO82442, 4198
PRO82444, 4201
PRO82446, 4206
PRO82448, 4212
PRO82450, 4217
PRO82453, 4223
PRO82454, 4227
PRO82455, 4229
PRO82456, 4231
PRO82457, 4233
PRO82458, 4235
PRO82460, 4238
PRO82461, 4240
PRO82465, 4247
PRO82466, 4249
PRO82469, 4253
PRO82470, 4255
PRO82472, 4258
PRO82473, 4264
PRO82475, 4271
PRO82477, 4276
PRO82479, 4280
PRO82482, 4284
PRO82485, 4288
PRO82487, 4291
PRO82489, 4294
PRO82491, 4297
PRO82492, 4299
PRO82493, 4303
PRO82495, 4308
PRO82499, 4313
PRO82501, 4316
PRO82502, 4318
PRO82505, 4324
PRO82508, 4328
PRO82509, 4330
PRO82510, 4332
PRO82513, 4339
PRO82514, 4341
PRO82515, 4345
PRO82516, 4347
PRO82517, 4349
PRO82518, 4351
PRO82521, 4365
PRO82522, 4367
PRO82523, 4369
PRO82524, 4377
PRO82525, 4379
PRO82526, 4381
PRO82527, 4383
PRO82528, 4385
PRO82529, 4387
PRO82530, 4389
PRO82531, 4391
PRO82532, 4393
PRO82533, 4395
PRO82534, 4397
PRO82535, 4401
PRO82536, 4403
PRO82537, 4405
PRO82538, 4407
PRO82540, 4412
PRO82542, 4415
PRO82543, 4417
PRO82544, 4419
PRO82546, 4424
PRO82548, 4428
PRO82551, 4433
PRO82554, 4437
PRO82555, 4439
PRO82556, 4441
PRO82557, 4443
PRO82558, 4445
PRO82560, 4452
PRO82562, 4455
PRO82563, 4457
PRO82564, 4459
PRO82567, 4464
PRO82568, 4466
PRO82570, 4469
PRO82571, 4471
PRO82572, 4473
PRO82573, 4475
PRO82576, 4481
PRO82579, 4486
PRO82582, 4492
PRO82583, 4494
PRO82584, 4496
PRO82585, 4499
PRO82586, 4501
PRO82587, 4503
PRO82589, 4506
PRO82590, 4510
PRO82592, 4515
PRO82593, 4523
PRO82594, 4525
PRO82597, 4529
PRO82598, 4533
PRO82599, 4536
PRO82602, 4540
PRO82603, 4542
PRO82606, 4546
PRO82607, 4548
PRO82608, 4552
PRO82609, 4554
PRO82611, 4557
PRO82612, 4559
PRO82615, 4563
PRO82616, 4567
PRO82618, 4572
PRO82619, 4574
PRO82621, 4585
PRO82622, 4587
PRO82623, 4589
PRO82624, 4591
PRO82625, 4593
PRO82626, 4595
PRO82627, 4598
PRO82629, 4601
PRO82630, 4603
PRO82631, 4605
PRO82632, 4607
PRO82633, 4609
PRO82634, 4611
PRO82635, 4615
PRO82637, 4618
PRO82638, 4620
PRO82640, 4623
PRO82641, 4625
PRO82642, 4629
PRO82643, 4631
PRO82645, 4634
PRO82646, 4636
PRO82654, 4649
PRO82656, 4652
PRO82658, 4655
PRO82659, 4657
PRO82661, 4660
PRO82662, 4662
PRO82663, 4664
PRO82664, 4668
PRO82665, 4673
PRO82667, 4676
PRO82669, 4679
PRO82670, 4681
PRO82671, 4683
PRO82672, 4685
PRO82674, 4690
PRO82675, 4692
PRO82678, 4698
PRO82679, 4702
PRO82683, 4711
PRO82687, 4722
PRO82689, 4726
PRO82691, 4731
PRO82692, 4733
PRO82694, 4736
PRO82695, 4738
PRO82696, 4740
PRO82699, 4746
PRO82702, 4753
PRO82704, 4756
PRO82706, 4759
PRO82707, 4762
PRO82708, 4764
PRO82709, 4766
PRO82712, 4770
PRO82713, 4772
PRO82714, 4774
PRO82715, 4776
PRO82717, 4781
PRO82718, 4785
PRO82719, 4787
PRO82720, 4789
PRO82721, 4791
PRO82722, 4795
PRO82724, 4802
PRO82725, 4804
PRO82726, 4808
PRO82728, 4811
PRO82729, 4815
PRO82730, 4817
PRO82732, 4823
PRO82736, 4828
PRO82737, 4831
PRO82738, 4833
PRO82739, 4835
PRO82740, 4837
PRO82741, 4839
PRO82743, 4848
PRO82745, 4851
PRO82746, 4853
PRO82748, 4858
PRO82749, 4860
PRO82750, 4862
PRO82753, 4866
PRO82754, 4868
PRO82755, 4870
PRO82756, 4872
PRO82757, 4874
PRO82758, 4876
PRO82760, 4882
PRO82761, 4884
PRO82762, 4886
PRO82763, 4894
PRO82764, 4896
PRO82768, 4907
PRO82769, 4909
PRO82771, 4914
PRO82774, 4922
PRO82776, 4925
PRO82778, 4930
PRO82779, 4932
PRO82787, 4947
PRO82788, 4949
PRO82790, 4952
PRO82791, 4956
PRO82792, 4959
PRO82793, 4961
PRO82794, 4966
PRO82795, 4968
PRO82796, 4972
PRO82797, 4974
PRO82799, 4979
PRO82800, 4981
PRO82805, 4993
PRO82807, 4997
PRO82812, 5005
PRO82813, 5007
PRO82814, 5009
PRO82816, 5012
PRO82818, 5017
PRO82825, 5029
PRO82828, 5033
PRO82829, 5035
PRO82831, 5038
PRO82833, 5044
PRO82835, 5047
PRO82840, 5059
PRO82841, 5061
PRO82842, 5063
PRO82846, 5068
PRO82850, 5077
PRO82851, 5079
PRO82852, 5081
PRO82855, 5085
PRO82856, 5087
PRO82859, 5091
PRO82861, 5099
PRO82862, 5101
PRO82863, 5105
PRO82864, 5107
PRO82867, 5111
PRO82871, 5118
PRO82872, 5121
PRO82873, 5125
PRO82874, 5129
PRO82877, 5136
PRO82879, 5139
PRO82881, 5142
PRO82882, 5144
PRO82884, 5149
PRO82885, 5151
PRO82886, 5153
PRO82887, 5156
PRO82888, 5158
PRO82892, 5167
PRO82893, 5170
PRO82894, 5172
PRO82895, 5174
PRO82897, 5177
PRO82899, 5182
PRO82901, 5185
PRO82902, 5187
PRO82903, 5189
PRO82904, 5191
PRO82905, 5193
PRO82909, 5198
PRO82910, 5204
PRO82912, 5211
PRO82915, 5215
PRO82917, 5220
PRO82920, 5224
PRO82923, 5228
PRO82925, 5231
PRO82930, 5245
PRO82933, 5249
PRO82934, 5253
PRO82935, 5255
PRO82939, 5263
PRO82940, 5265
PRO82943, 5271
PRO82944, 5275
PRO82947, 5287
PRO82948, 5289
PRO82949, 5291
PRO82950, 5293
PRO82952, 5296
PRO82954, 5301
PRO82956, 5304
PRO82957, 5306
PRO82958, 5309
PRO82962, 5318
PRO82963, 5320
PRO82964, 5322
PRO82965, 5324
PRO82967, 5327
PRO82970, 5333
PRO82971, 5335
PRO82975, 5340
PRO82976, 5342
PRO82977, 5344
PRO82978, 5346
PRO82979, 5348
PRO82980, 5351
PRO82982, 5356
PRO82983, 5359
PRO82984, 5363
PRO82985, 5365
PRO82987, 5368
PRO82991, 5373
PRO82992, 5375
PRO82995, 5381
PRO82998, 5386
PRO82999, 5390
PRO83000, 5392
PRO83002, 5397
PRO83004, 5400
PRO83005, 5402
PRO83007, 5405
PRO83008, 5410
PRO83009, 5412
PRO83010, 5414
PRO83011, 5416
PRO83012, 5418
PRO83013, 5420
PRO83014, 5424
PRO83016, 5431
PRO83017, 5433
PRO83018, 5437
PRO83027, 5452
PRO83029, 5455
PRO83030, 5457
PRO83031, 5459
PRO83035, 5464
PRO83037, 5467
PRO83038, 5469
PRO83039, 5471
PRO83040, 5473
PRO83041, 5475
PRO83042, 5481
PRO83050, 5493
PRO83052, 5500
PRO83054, 5509
PRO83056, 5520
PRO83059, 5526
PRO83065, 5533
PRO83066, 5535
PRO83068, 5538
PRO83069, 5540
PRO83071, 5545
PRO83072, 5547
PRO83073, 5553
PRO83074, 5555
PRO83075, 5557
PRO83076, 5559
PRO83077, 5561
PRO83078, 5567
PRO83080, 5572
PRO83082, 5579
PRO83083, 5581
PRO83084, 5583
PRO83085, 5585
PRO83086, 5587
PRO83087, 5596
PRO83089, 5599
PRO83090, 5601
PRO83092, 5604
PRO83093, 5606
PRO83095, 5609
PRO83096, 5611
PRO83098, 5614
PRO83099, 5618
PRO83100, 5620
PRO83101, 5622
PRO83102, 5624
PRO83103, 5630
PRO83104, 5632
PRO83105, 5634
PRO83107, 5637
PRO83108, 5643
PRO83109, 5645
PRO83112, 5653
PRO83113, 5657
PRO83114, 5659
PRO83116, 5662
PRO83117, 5664
PRO83118, 5666
PRO83121, 5672
PRO83125, 5684
PRO83128, 5690
PRO83129, 5692
PRO83130, 5694
PRO83132, 5697
PRO83133, 5699
PRO83135, 5702
PRO83137, 5707
PRO83138, 5709
PRO83139, 5711
PRO83141, 5714
PRO83142, 5716
PRO83143, 5718
PRO83144, 5720
PRO83145, 5724
PRO83146, 5726
PRO83149, 5730
PRO83150, 5732
PRO83152, 5737
PRO83153, 5741
PRO83155, 5751
PRO83156, 5753
PRO83157, 5755
PRO83159, 5758
PRO83161, 5764
PRO83163, 5767
PRO83165, 5770
PRO83167, 5777
PRO83169, 5784
PRO83170, 5788
PRO83174, 5797
PRO83175, 5799
PRO83176, 5801
PRO83177, 5803
PRO83178, 5805
PRO83179, 5807
PRO83180, 5812
PRO83182, 5817
PRO83183, 5820
PRO83184, 5822
PRO83185, 5827
PRO83186, 5829
PRO83187, 5833
PRO83188, 5835
PRO83189, 5839
PRO83190, 5841
PRO83191, 5843
PRO83193, 5848
PRO83194, 5850
PRO83195, 5852
PRO83196, 5854
PRO83197, 5856
PRO83198, 5858
PRO83199, 5860
PRO83200, 5862
PRO83201, 5864
PRO83202, 5866
PRO83203, 5868
PRO83204, 5870
PRO83205, 5872
PRO83210, 5884
PRO83211, 5888
PRO83212, 5895
PRO83213, 5899
PRO83214, 5901
PRO83217, 5909
PRO83219, 5912
PRO83222, 5916
PRO83223, 5918
PRO83224, 5920
PRO83233, 5932
PRO83234, 5934
PRO83235, 5938
PRO83236, 5942
PRO83237, 5944
PRO83242, 5952
PRO83244, 5955
PRO83245, 5959
PRO83247, 5962
PRO83252, 5970
PRO83253, 5972
PRO83254, 5978
PRO83255, 5980
PRO83256, 5982
PRO83257, 5984
PRO83260, 5988
PRO83261, 5992
PRO83263, 5997
PRO83265, 6002
PRO83266, 6008
PRO83267, 6010
PRO83270, 6016
PRO83271, 6018
PRO83273, 6026
PRO83274, 6033
PRO83275, 6037
PRO83276, 6041
PRO83278, 6044
PRO83279, 6048
PRO83280, 6050
PRO83282, 6053
PRO83283, 6057
PRO83285, 6062
PRO83288, 6067
PRO83289, 6073
PRO83291, 6078
PRO83292, 6084
PRO83293, 6086
PRO83297, 6092
PRO83300, 6096
PRO83301, 6098
PRO83302, 6100
PRO83304, 6107
PRO83306, 6110
PRO83307, 6112
PRO83309, 6115
PRO83310, 6117
PRO83312, 6120
PRO83316, 6129
PRO83319, 6133
PRO83320, 6135
PRO83321, 6137
PRO83323, 6144
PRO83328, 6156
PRO83331, 6162
PRO83332, 6164
PRO83333, 6166
PRO83334, 6168
PRO83335, 6171
PRO83337, 6175
PRO83339, 6178
PRO83340, 6180
PRO83341, 6184
PRO83343, 6193
PRO83344, 6195
PRO83345, 6200
PRO83346, 6202
PRO83349, 6208
PRO83351, 6211
PRO83352, 6213
PRO83353, 6219
PRO83354, 6221
PRO83355, 6223
PRO83360, 6234
PRO83361, 6236
PRO83365, 6247
PRO83366, 6249
PRO83368, 6252
PRO83369, 6254
PRO83372, 6260
PRO83373, 6262
PRO83374, 6264
PRO83375, 6266
PRO83381, 6277
PRO83383, 6283
PRO83385, 6290
PRO83386, 6292
PRO83387, 6294
PRO83388, 6296
PRO83389, 6298
PRO83391, 6301
PRO83392, 6303
PRO83393, 6305
PRO83394, 6307
PRO83395, 6309
PRO83397, 6314
PRO83400, 6324
PRO83403, 6331
PRO83404, 6337
PRO83405, 6347
PRO868, 1871
PRO9112, 3668
PRO9785, 1369
PRO9819, 2676
PRO983, 5825
PRO9886, 706
PRO9902, 2952
PRO9980, 2479
PRO9984, 969
PRO9987, 3753

Accession Index (to Figure number)

NM_000018, 4669
NM_000026, 6068
NM_000029, 624
NM_000033, 6342
NM_000034, 4520
NM_000039, 3376
NM_000041, 5511
NM_000070, 4161
NM_000075, 3683
NM_000077, 2655
NM_000079, 898
NM_000090, 921
NM_000107, 3208
NM_000114, 5836
NM_000121, 5258
NM_000126, 4267
NM_000137, 4300
NM_000143, 636
NM_000146, 5562
NM_000154, 4967
NM_000156, 5122
NM_000165, 2099
NM_000177, 2796
NM_000178, 5738
NM_000179, 744
NM_000182, 713
NM_000183, 711
NM_000184, 3144
NM_000196, 4547
NM_000213, 4963
NM_000221, 701
NM_000224, 3593
NM_000227, 5040
NM_000228, 553
NM_000239, 3729
NM_000250, 4903
NM_000251, 741
NM_000268, 5994
NM_000269, 4889
NM_000274, 3076
NM_000284, 6138
NM_000291, 6230
NM_000358, 1671
NM_000365, 3460
NM_000368, 2806
NM_000385, 2262
NM_000386, 4843
NM_000396, 356
NM_000404, 1089
NM_000407, 5947
NM_000422, 4807
NM_000425, 6334
NM_000447, 594
NM_000484, 5882
NM_000505, 1828
NM_000508, 1511
NM_000509, 1515
NM_000516, 5830
NM_000517, 4354
NM_000521, 1627
NM_000526, 4816
NM_000532, 1260
NM_000554, 5480
NM_000558, 4356
NM_000559, 3142
NM_000569, 505
NM_000574, 558
NM_000576, 847
NM_000582, 1459
NM_000592, 1957
NM_000598, 2228
NM_000602, 2361
NM_000612, 3120
NM_000638, 4763
NM_000661, 1425
NM_000666, 1172
NM_000687, 5736
NM_000688, 1167
NM_000700, 2695
NM_000701, 312
NM_000743, 4259
NM_000754, 5956
NM_000760, 173
NM_000785, 3687
NM_000787, 2830
NM_000795, 3384
NM_000801, 5648
NM_000852, 3297
NM_000858, 612
NM_000893, 1327
NM_000895, 3763
NM_000930, 2534
NM_000931, 2536
NM_000942, 4218
NM_000954, 2868
NM_000964, 4820
NM_000967, 6061
NM_000969, 284
NM_000970, 3781
NM_000971, 2569
NM_000972, 2826
NM_000973, 2633
NM_000975, 87
NM_000976, 2780
NM_000977, 4633
NM_000978, 4801
NM_000979, 5571
NM_000980, 5334
NM_000981, 4798
NM_000982, 3091
NM_000983, 34
NM_000985, 5067
NM_000986, 1206
NM_000987, 4714
NM_000989, 2588
NM_000990, 3155
NM_000991, 5613
NM_000992, 1170
NM_000993, 832
NM_000994, 1064
NM_000997, 1570
NM_000998, 966
NM_001000, 6278
NM_001002, 3827
NM_001003, 4228
NM_001005, 3331
NM_001006, 1506
NM_001007, 6224
NM_001009, 5633
NM_001010, 2651
NM_001011, 643
NM_001012, 210
NM_001016, 2111
NM_001017, 3171
NM_001018, 5126
NM_001020, 5426
NM_001021, 4283
NM_001022, 5468
NM_001023, 2552
NM_001024, 5847
NM_001025, 1632
NM_001026, 2980
NM_001028, 3361
NM_001029, 3656
NM_001030, 440
NM_001034, 651
NM_001038, 3478
NM_001043, 4487
NM_001050, 4841
NM_001064, 1159
NM_001065, 3480
NM_001068, 1079
NM_001069, 2050
NM_001084, 2369
NM_001087, 994
NM_001098, 6079
NM_001101, 2174
NM_001102, 4040
NM_001122, 2649
NM_001134, 1446
NM_001154, 1489
NM_001157, 2990
NM_001168, 4985
NM_001190, 5568
NM_001199, 2495
NM_001207, 1624
NM_001211, 4139
NM_001218, 4203
NM_001235, 3333
NM_001238, 5374
NM_001247, 5703
NM_001255, 194
NM_001262, 229
NM_001273, 3468
NM_001274, 3411
NM_001275, 4065
NM_001283, 2365
NM_001287, 4372
NM_001288, 1969
NM_001293, 3337
NM_001294, 5508
NM_001313, 1396
NM_001319, 5141
NM_001320, 1971
NM_001324, 5814
NM_001325, 6239
NM_001333, 2736
NM_001344, 3984
NM_001350, 1942
NM_001363, 6318
NM_001407, 1132
NM_001415, 6143
NM_001416, 4687
NM_001418, 3163
NM_001428, 31
NM_001436, 5436
NM_001444, 2575
NM_001450, 836
NM_001463, 916
NM_001465, 1573
NM_001467, 3359
NM_001469, 6081
NM_001494, 2891
NM_001500, 2052
NM_001517, 1997
NM_001521, 689
NM_001530, 4016
NM_001536, 5539
NM_001539, 2660
NM_001540, 2308
NM_001553, 1435
NM_001554, 269
NM_001560, 6270
NM_001567, 3322
NM_001568, 2596
NM_001569, 6332
NM_001571, 5542
NM_001605, 4564
NM_001607, 1097
NM_001610, 3206
NM_001613, 3008
NM_001622, 1330
NM_001628, 2423
NM_001641, 3997
NM_001644, 3511
NM_001647, 1352
NM_001648, 5590
NM_001659, 3550
NM_001662, 2398
NM_001667, 3284
NM_001673, 2355
NM_001687, 5115
NM_001688, 308
NM_001696, 5941
NM_001697, 5892
NM_001710, 1959
NM_001734, 3452
NM_001743, 5494
NM_001747, 806
NM_001751, 3137
NM_001753, 2391
NM_001757, 5894
NM_001760, 1898
NM_001762, 2274
NM_001780, 3663
NM_001791, 81
NM_001816, 5478
NM_001819, 5679
NM_001827, 2714
NM_001831, 2506
NM_001833, 2689
NM_001842, 2668
NM_001853, 5853
NM_001861, 4614
NM_001862, 827
NM_001878, 392
NM_001907, 4579
NM_001909, 3133
NM_001920, 3740
NM_001930, 5267
NM_001935, 894
NM_001944, 5050
NM_001959, 950
NM_001961, 5178
NM_001964, 1689
NM_001969, 4098
NM_001970, 4697
NM_001975, 3458
NM_001983, 5502
NM_001985, 5593
NM_002003, 2834
NM_002004, 422
NM_002011, 1836
NM_002014, 3439
NM_002015, 3896
NM_002018, 4719
NM_002028, 4010
NM_002046, 3473
NM_002047, 2265
NM_002075, 3463
NM_002079, 3066
NM_002083, 4012
NM_002084, 1704
NM_002085, 5112
NM_002086, 4953
NM_002087, 4845
NM_002106, 1478
NM_002109, 1779
NM_002128, 3887
NM_002129, 1522
NM_002130, 1582
NM_002133, 6020
NM_002137, 2210
NM_002157, 930
NM_002161, 2716
NM_002168, 4293
NM_002178, 3600
NM_002211, 2919
NM_002212, 5742
NM_002229, 5272
NM_002265, 4834
NM_002273, 3591
NM_002274, 4814
NM_002275, 4812
NM_002276, 4810
NM_002295, 1108
NM_002305, 6038
NM_002306, 4022
NM_002339, 3115
NM_002340, 5931
NM_002342, 3476
NM_002345, 3752
NM_002355, 3489
NM_002358, 1485
NM_002364, 6147
NM_002385, 5086
NM_002386, 4626
NM_002388, 1866
NM_002396, 5069
NM_002397, 1646
NM_002401, 4933
NM_002411, 3245
NM_002413, 1494
NM_002414, 6124
NM_002415, 5979
NM_002453, 751
NM_002466, 5774
NM_002468, 1095
NM_002473, 6025
NM_002477, 1368
NM_002484, 4416
NM_002486, 2734
NM_002489, 2193
NM_002492, 1297
NM_002512, 4887
NM_002520, 1803
NM_002537, 4210
NM_002539, 659
NM_002567, 3816
NM_002568, 2593
NM_002574, 220
NM_002588, 1728
NM_002606, 5900
NM_002615, 4647
NM_002617, 12
NM_002632, 4052
NM_002634, 4939
NM_002638, 5779
NM_002654, 4242
NM_002660, 5771
NM_002668, 6185
NM_002689, 3289
NM_002691, 5580
NM_002707, 681
NM_002712, 1030
NM_002720, 4518
NM_002727, 2961
NM_002730, 5298
NM_002733, 3555
NM_002766, 4975
NM_002787, 2254
NM_002789, 4261
NM_002792, 5838
NM_002793, 2137
NM_002796, 346
NM_002802, 4059
NM_002803, 2378
NM_002809, 4805
NM_002810, 348
NM_002812, 5401
NM_002813, 3837
NM_002815, 4778
NM_002819, 5102
NM_002827, 5809
NM_002846, 980
NM_002854, 1188
NM_002856, 5515
NM_002857, 481
NM_002863, 4029
NM_002870, 438
NM_002878, 4784
NM_002883, 6075
NM_002887, 1800
NM_002913, 1427
NM_002915, 3891
NM_002921, 3002
NM_002923, 540
NM_002934, 3992
NM_002938, 1386
NM_002946, 127
NM_002947, 2188
NM_002948, 1076
NM_002952, 4382
NM_002954, 749
NM_002961, 369
NM_002965, 364
NM_002979, 235
NM_003002, 3390
NM_003021, 5161
NM_003025, 5188
NM_003055, 2947
NM_003064, 5781
NM_003072, 5254
NM_003076, 3568
NM_003088, 2176
NM_003090, 4320
NM_003091, 5654
NM_003092, 5683
NM_003104, 4187
NM_003107, 2032
NM_003123, 4511
NM_003124, 789
NM_003128, 746
NM_003132, 50
NM_003137, 1916
NM_003143, 2435
NM_003145, 409
NM_003146, 3215
NM_003149, 1099
NM_003169, 5428
NM_003181, 2135
NM_003216, 6077
NM_003283, 5608
NM_003287, 2104
NM_003289, 2680
NM_003290, 5312
NM_003295, 3900
NM_003310, 649
NM_003316, 5896
NM_003334, 6167
NM_003349, 5804
NM_003350, 2546
NM_003365, 1134
NM_003366, 4421
NM_003370, 5499
NM_003374, 1677
NM_003375, 2982
NM_003378, 2367
NM_003389, 2728
NM_003400, 761
NM_003401, 1636
NM_003406, 2590
NM_003418, 1250
NM_003453, 3864
NM_003461, 2440
NM_003472, 2034
NM_003516, 459
NM_003564, 474
NM_003598, 5556
NM_003617, 497
NM_003624, 5214
NM_003626, 3316
NM_003646, 3197
NM_003662, 6149
NM_003680, 157
NM_003681, 5905
NM_003685, 5203
NM_003687, 1673
NM_003689, 71
NM_003712, 5093
NM_003714, 1812
NM_003720, 5898
NM_003721, 5360
NM_003722, 1335
NM_003729, 288
NM_003735, 1730
NM_003736, 1732
NM_003739, 2883
NM_003752, 4449
NM_003753, 6027
NM_003755, 5234
NM_003756, 2598
NM_003757, 148
NM_003765, 5288
NM_003766, 4865
NM_003779, 468
NM_003780, 199
NM_003787, 5052
NM_003815, 457
NM_003824, 3313
NM_003836, 4088
NM_003837, 2723
NM_003859, 5811
NM_003876, 4708
NM_003877, 3757
NM_003906, 5933
NM_003908, 5734
NM_003915, 5747
NM_003932, 6070
NM_003937, 881
NM_003938, 5148
NM_003971, 4891
NM_003973, 1110
NM_003979, 3498
NM_004000, 306
NM_004004, 3866
NM_004044, 955
NM_004048, 4178
NM_004053, 1900
NM_004060, 1791
NM_004074, 3264
NM_004084, 2476
NM_004085, 6242
NM_004092, 3099
NM_004111, 3253
NM_004117, 1918
NM_004127, 5008
NM_004134, 1693
NM_004135, 6340
NM_004147, 6011
NM_004152, 5154
NM_004159, 1952
NM_004175, 5983
NM_004176, 4742
NM_004178, 3614
NM_004181, 1430
NM_004182, 6174
NM_004193, 3045
NM_004203, 4402
NM_004208, 6285
NM_004217, 4699
NM_004219, 1795
NM_004240, 5206
NM_004247, 4879
NM_004261, 273
NM_004265, 3249
NM_004309, 5002
NM_004322, 3256
NM_004323, 2662
NM_004324, 5564
NM_004335, 5328
NM_004339, 5921
NM_004341, 692
NM_004345, 1128
NM_004360, 4549
NM_004398, 3392
NM_004401, 48
NM_004404, 1034
NM_004435, 2761
NM_004448, 4796
NM_004461, 5279
NM_004483, 4602
NM_004493, 6190
NM_004509, 1012
NM_004510, 1014
NM_004524, 4960
NM_004539, 5072
NM_004547, 1218
NM_004550, 470
NM_004551, 3199
NM_004555, 4586
NM_004573, 4141
NM_004595, 6140
NM_004596, 5448
NM_004599, 6085
NM_004618, 4716
NM_004632, 414
NM_004635, 1155
NM_004636, 1149
NM_004637, 1246
NM_004638, 1979
NM_004639, 1973
NM_004640, 1986
NM_004673, 529
NM_004691, 4545
NM_004697, 2751
NM_004699, 6323
NM_004701, 4197
NM_004704, 1182
NM_004706, 5470
NM_004714, 5434
NM_004725, 3093
NM_004728, 2959
NM_004735, 1026
NM_004738, 5824
NM_004739, 3230
NM_004766, 1270
NM_004767, 576
NM_004772, 1650
NM_004781, 44
NM_004794, 6287
NM_004813, 3190
NM_004821, 1787
NM_004844, 1066
NM_004846, 998
NM_004859, 4921
NM_004870, 4689
NM_004889, 2342
NM_004893, 1685
NM_004905, 511
NM_004911, 2442
NM_004928, 5915
NM_004930, 69
NM_004933, 4638
NM_004939, 662
NM_004957, 2775
NM_004960, 4465
NM_004964, 150
NM_004973, 2039
NM_004982, 3526
NM_004990, 3669
NM_004992, 6330
NM_004994, 5791
NM_004995, 3976
NM_005000, 2396
NM_005002, 3448
NM_005003, 4446
NM_005004, 3063
NM_005005, 2606
NM_005008, 6083
NM_005015, 3981
NM_005016, 3620
NM_005022, 4665
NM_005030, 4442
NM_005036, 6104
NM_005042, 3524
NM_005053, 5283
NM_005072, 4581
NM_005080, 5987
NM_005109, 1093
NM_005110, 1854
NM_005112, 1421
NM_005115, 4500
NM_005132, 3962
NM_005141, 1508
NM_005163, 4110
NM_005171, 3574
NM_005174, 2895
NM_005194, 5808
NM_005217, 2478
NM_005220, 4946
NM_005224, 5104
NM_005243, 5989
NM_005269, 3667
NM_005271, 3004
NM_005291, 854
NM_005300, 6159
NM_005313, 4174
NM_005324, 4969
NM_005330, 3146
NM_005333, 6126
NM_005345, 1963
NM_005346, 1961
NM_005347, 2790
NM_005348, 4092
NM_005362, 6316
NM_005364, 6308
NM_005370, 5314
NM_005371, 3689
NM_005378, 657
NM_005389, 2126
NM_005432, 4101
NM_005439, 3466
NM_005440, 4877
NM_005452, 1944
NM_005474, 4850
NM_005490, 5208
NM_005498, 5241
NM_005514, 2155
NM_005517, 110
NM_005520, 1850
NM_005548, 4568
NM_005563, 105
NM_005566, 3175
NM_005572, 404
NM_005573, 1718
NM_005581, 5517
NM_005594, 3628
NM_005614, 2460
NM_005617, 1708
NM_005620, 340
NM_005623, 4782
NM_005632, 4362
NM_005657, 4170
NM_005663, 1382
NM_005676, 6165
NM_005686, 550
NM_005692, 2458
NM_005693, 3204
NM_005698, 424
NM_005710, 6181
NM_005713, 1602
NM_005717, 517
NM_005718, 1055
NM_005720, 2348
NM_005724, 4273
NM_005726, 3695
NM_005729, 2986
NM_005731, 996
NM_005745, 6344
NM_005754, 1697
NM_005762, 5627
NM_005770, 4176
NM_005775, 2491
NM_005783, 829
NM_005787, 1316
NM_005796, 4575
NM_005806, 5887
NM_005826, 83
NM_005830, 3898
NM_005831, 4911
NM_005833, 2792
NM_005837, 2326
NM_005850, 461
NM_005851, 3301
NM_005855, 1024
NM_005866, 2670
NM_005877, 5999
NM_005884, 5421
NM_005889, 3509
NM_005911, 808
NM_005915, 864
NM_005917, 764
NM_005918, 2306
NM_005973, 389
NM_005981, 3681
NM_005983, 1579
NM_005985, 5802
NM_005997, 350
NM_006000, 982
NM_006012, 5201
NM_006013, 6326
NM_006019, 3304
NM_006023, 2899
NM_006039, 4936
NM_006053, 3306
NM_006058, 1702
NM_006066, 218
NM_006067, 4612
NM_006098, 1852
NM_006101, 5023
NM_006109, 3973
NM_006110, 4423
NM_006112, 159
NM_006114, 5513
NM_006115, 5975
NM_006128, 2497
NM_006131, 2499
NM_006132, 2501
NM_006136, 2393
NM_006169, 3380
NM_006184, 5566
NM_006227, 5789
NM_006230, 2246
NM_006245, 1892
NM_006247, 5497
NM_006250, 3522
NM_006253, 3831
NM_006262, 3546
NM_006265, 2600
NM_006271, 374
NM_006272, 5935
NM_006280, 6338
NM_006289, 2682
NM_006295, 1967
NM_006303, 2178
NM_006330, 2550
NM_006335, 571
NM_006339, 5171
NM_006342, 1374
NM_006349, 2371
NM_006354, 1049
NM_006362, 3242
NM_006365, 396
NM_006373, 4875
NM_006384, 4305
NM_006387, 5319
NM_006395, 1062
NM_006397, 5277
NM_006401, 2732
NM_006427, 4106
NM_006428, 4360
NM_006429, 792
NM_006430, 759
NM_006432, 4048
NM_006435, 3113
NM_006439, 1504
NM_006440, 5954
NM_006453, 4384
NM_006455, 4822
NM_006470, 4725
NM_006478, 5991
NM_006488, 703
NM_006494, 5476
NM_006503, 5441
NM_006513, 298
NM_006516, 188
NM_006523, 3055
NM_006530, 3727
NM_006556, 452
NM_006559, 146
NM_006576, 3697
NM_006585, 5885
NM_006586, 1894
NM_006589, 428
NM_006600, 118
NM_006601, 3636
NM_006621, 300
NM_006625, 93
NM_006636, 794
NM_006646, 3881
NM_006659, 3101
NM_006666, 5558
NM_006667, 6272
NM_006670, 2070
NM_006693, 2344
NM_006694, 436
NM_006698, 5760
NM_006708, 1904
NM_006711, 4392
NM_006746, 6134
NM_006761, 4642
NM_006763, 548
NM_006764, 1151
NM_006769, 271
NM_006787, 6197
NM_006791, 4279
NM_006799, 4408
NM_006801, 5576
NM_006805, 1687
NM_006808, 2740
NM_006810, 1223
NM_006812, 3678
NM_006815, 3847
NM_006816, 1830
NM_006817, 3785
NM_006821, 4046
NM_006824, 192
NM_006825, 3807
NM_006826, 655
NM_006833, 2338
NM_006835, 1449
NM_006837, 2565
NM_006839, 814
NM_006842, 3295
NM_006844, 5308
NM_006854, 2184
NM_006862, 344
NM_006888, 4063
NM_006899, 5661
NM_006908, 2182
NM_006924, 4908
NM_006928, 3660
NM_006932, 6007
NM_006938, 5039
NM_006941, 6049
NM_006942, 4691
NM_006990, 124
NM_007002, 5844
NM_007019, 5785
NM_007032, 6040
NM_007034, 267
NM_007046, 705
NM_007047, 2029
NM_007062, 3805
NM_007065, 5237
NM_007074, 4516
NM_007085, 1216
NM_007096, 2691
NM_007100, 1366
NM_007103, 3299
NM_007104, 1922
NM_007158, 302
NM_007165, 5152
NM_007173, 3348
NM_007178, 3501
NM_007184, 1165
NM_007186, 5744
NM_007190, 3089
NM_007209, 2794
NM_007242, 4566
NM_007244, 3520
NM_007260, 89
NM_007262, 42
NM_007263, 5352
NM_007268, 6204
NM_007273, 3455
NM_007275, 1153
NM_007276, 2214
NM_007279, 5619
NM_007310, 5958
NM_007311, 6095
NM_007317, 4507
NM_007355, 1874
NM_007364, 4277
NM_007372, 4931
NM_012068, 5525
NM_012098, 2782
NM_012099, 5504
NM_012100, 977
NM_012101, 3420
NM_012111, 4055
NM_012112, 5715
NM_012116, 5519
NM_012138, 4838
NM_012170, 4265
NM_012179, 6017
NM_012181, 5350
NM_012203, 2693
NM_012207, 2955
NM_012237, 5409
NM_012248, 4451
NM_012255, 5698
NM_012264, 6054
NM_012286, 6246
NM_012296, 3344
NM_012323, 6052
NM_012391, 1929
NM_012412, 2236
NM_012423, 5550
NM_012437, 381
NM_012458, 5155
NM_012469, 5873
NM_012486, 596
NM_013237, 1834
NM_013247, 801
NM_013265, 3279
NM_013274, 3037
NM_013277, 3566
NM_013296, 292
NM_013333, 5617
NM_013336, 1238
NM_013341, 903
NM_013363, 1276
NM_013365, 6032
NM_013369, 5911
NM_013375, 2027
NM_013393, 2165
NM_013402, 3251
NM_013403, 5492
NM_013406, 5269
NM_013407, 5270
NM_013417, 2718
NM_013442, 2675
NM_013451, 3013
NM_014003, 4592
NM_014008, 6187
NM_014033, 3576
NM_014035, 1664
NM_014042, 3320
NM_014062, 4556
NM_014063, 2251
NM_014107, 2077
NM_014138, 6163
NM_014166, 3906
NM_014172, 2862
NM_014173, 5326
NM_014176, 578
NM_014184, 585
NM_014188, 17
NM_014189, 1390
NM_014190, 1388
NM_014203, 5536
NM_014214, 5032
NM_014226, 4095
NM_014236, 626
NM_014248, 6072
NM_014255, 3631
NM_014267, 3173
NM_014275, 1846
NM_014285, 2820
NM_014294, 2567
NM_014303, 6003
NM_014306, 6015
NM_014311, 3606
NM_014320, 2116
NM_014321, 4476
NM_014325, 3777
NM_014335, 4182
NM_014341, 1906
NM_014353, 4386
NM_014408, 167
NM_014413, 2180
NM_014426, 5685
NM_014444, 4168
NM_014445, 1284
NM_014452, 1870
NM_014453, 5625
NM_014481, 6199
NM_014501, 5615
NM_014502, 3220
NM_014515, 3724
NM_014556, 1394
NM_014571, 142
NM_014585, 923
NM_014587, 4370
NM_014610, 3232
NM_014624, 367
NM_014649, 5199
NM_014663, 202
NM_014670, 934
NM_014685, 4530
NM_014713, 667
NM_014736, 4214
NM_014737, 5676
NM_014742, 5721
NM_014747, 180
NM_014748, 684
NM_014752, 3329
NM_014773, 1721
NM_014776, 3792
NM_014778, 3878
NM_014800, 2259
NM_014814, 1195
NM_014829, 1681
NM_014837, 519
NM_014847, 446
NM_014849, 463
NM_014851, 36
NM_014868, 3823
NM_014887, 3889
NM_014919, 1378
NM_014931, 5610
NM_014933, 1457
NM_014941, 6005
NM_014972, 4628
NM_015043, 1843
NM_015062, 3042
NM_015064, 3430
NM_015068, 2319
NM_015129, 6276
NM_015140, 6097
NM_015179, 3024
NM_015322, 4226
NM_015324, 3149
NM_015373, 6056
NM_015388, 1886
NM_015438, 3470
NM_015449, 444
NM_015453, 1043
NM_015472, 1282
NM_015484, 99
NM_015511, 5752
NM_015533, 3225
NM_015544, 4780
NM_015584, 4761
NM_015629, 5600
NM_015636, 686
NM_015640, 260
NM_015644, 1057
NM_015646, 3720
NM_015665, 3604
NM_015702, 885
NM_015714, 555
NM_015853, 3238
NM_015920, 4205
NM_015932, 3884
NM_015934, 941
NM_015937, 5783
NM_015953, 5546
NM_015965, 5362
NM_015966, 5745
NM_016003, 2172
NM_016016, 4847
NM_016022, 334
NM_016026, 4037
NM_016030, 647
NM_016059, 1908
NM_016085, 694
NM_016091, 6045
NM_016095, 4610
NM_016111, 4374
NM_016119, 3912
NM_016143, 5652
NM_016169, 3051
NM_016174, 2767
NM_016176, 26
NM_016183, 73
NM_016202, 5621
NM_016223, 3210
NM_016249, 6300
NM_016263, 5169
NM_016267, 6293
NM_016286, 5006
NM_016292, 4414
NM_016304, 4193
NM_016328, 2293
NM_016357, 3572
NM_016359, 4152
NM_016361, 328
NM_016410, 2664
NM_016440, 5523
NM_016445, 4035
NM_016456, 564
NM_016498, 6001
NM_016526, 3107
NM_016539, 5181
NM_016558, 5750
NM_016567, 3097
NM_016579, 5216
NM_016587, 2216
NM_016592, 5826
NM_016638, 3843
NM_016639, 4398
NM_016641, 4335
NM_016645, 4302
NM_016647, 2614
NM_016732, 5733
NM_016838, 887
NM_016839, 889
NM_016930, 1400
NM_016940, 5883
NM_016941, 5432
NM_017443, 2753
NM_017458, 4498
NM_017491, 1419
NM_017546, 834
NM_017566, 4617
NM_017572, 5146
NM_017595, 4871
NM_017601, 1902
NM_017610, 4195
NM_017613, 5890
NM_017647, 4929
NM_017668, 4327
NM_017670, 3266
NM_017684, 4208
NM_017722, 5286
NM_017751, 859
NM_017760, 2467
NM_017761, 91
NM_017768, 262
NM_017777, 4906
NM_017789, 825
NM_017797, 5143
NM_017801, 1081
NM_017803, 4584
NM_017807, 4003
NM_017815, 3971
NM_017822, 3552
NM_017825, 165
NM_017827, 5413
NM_017829, 5939
NM_017847, 513
NM_017853, 4594
NM_017868, 3386
NM_017874, 5668
NM_017876, 5098
NM_017882, 4224
NM_017883, 6179
NM_017891, 8
NM_017895, 5798
NM_017900, 22
NM_017901, 3810
NM_017910, 674
NM_017916, 5554
NM_017952, 812
NM_017955, 4112
NM_017974, 1020
NM_018019, 4737
NM_018023, 1306
NM_018032, 4358
NM_018034, 1575
NM_018035, 5458
NM_018047, 1706
NM_018048, 3517
NM_018054, 4436
NM_018066, 116
NM_018070, 239
NM_018085, 569
NM_018096, 4792
NM_018110, 4535
NM_018113, 3548
NM_018116, 420
NM_018122, 535
NM_018124, 4588
NM_018135, 1880
NM_018154, 5300
NM_018174, 5332
NM_018188, 10
NM_018209, 5861
NM_018212, 587
NM_018217, 5740
NM_018238, 2437
NM_018242, 4747
NM_018250, 2510
NM_018253, 418
NM_018255, 5056
NM_018270, 5849
NM_018310, 2527
NM_018346, 4898
NM_018357, 4232
NM_018410, 1018
NM_018454, 4154
NM_018457, 3610
NM_018463, 3442
NM_018464, 2951
NM_018468, 5387
NM_018486, 6222
NM_018509, 4900
NM_018607, 721
NM_018660, 2512
NM_018668, 4312
NM_018674, 973
NM_018686, 3513
NM_018912, 1734
NM_018913, 1736
NM_018914, 1738
NM_018915, 1740
NM_018916, 1742
NM_018917, 1744
NM_018918, 1746
NM_018919, 1748
NM_018920, 1750
NM_018921, 1752
NM_018922, 1754
NM_018923, 1756
NM_018924, 1758
NM_018925, 1760
NM_018926, 1762
NM_018927, 1764
NM_018928, 1766
NM_018929, 1768
NM_018947, 2208
NM_018948, 41
NM_018950, 2017
NM_018955, 4728
NM_018957, 6034
NM_018977, 6214
NM_019013, 4682
NM_019058, 2971
NM_019059, 2206
NM_019082, 2242
NM_019095, 5681
NM_019099, 310
NM_019554, 371
NM_019606, 2333
NM_019609, 5663
NM_019619, 2916
NM_019848, 6321
NM_019852, 3988
NM_019887, 3839
NM_020037, 4895
NM_020038, 4893
NM_020132, 5908
NM_020134, 709
NM_020149, 4136
NM_020158, 5454
NM_020188, 4604
NM_020230, 5232
NM_020243, 6058
NM_020299, 2425
NM_020315, 6036
NM_020320, 2075
NM_020347, 1113
NM_020401, 3717
NM_020414, 4069
NM_020418, 1180
NM_020548, 871
NM_020675, 896
NM_020677, 4340
NM_020701, 1248
NM_020990, 4172
NM_020992, 3017
NM_021019, 3646
NM_021029, 6244
NM_021079, 4883
NM_021095, 698
NM_021103, 803
NM_021104, 3654
NM_021107, 5415
NM_021121, 948
NM_021126, 6029
NM_021129, 2964
NM_021130, 2238
NM_021141, 958
NM_021154, 2701
NM_021158, 5638
NM_021177, 1965
NM_021178, 4006
NM_021195, 4400
NM_021213, 4919
NM_021219, 5879
NM_021226, 2945
NM_021626, 4917
NM_021709, 4108
NM_021728, 4020
NM_021826, 5665
NM_021830, 3033
NM_021831, 707
NM_021870, 1517
NM_021871, 1513
NM_021932, 3109
NM_021934, 3588
NM_021948, 394
NM_021953, 3444
NM_021966, 4079
NM_021999, 3908
NM_022003, 3369
NM_022039, 3039
NM_022044, 5973
NM_022048, 4216
NM_022105, 5857
NM_022137, 4042
NM_022141, 6101
NM_022158, 5016
NM_022170, 2288
NM_022171, 1145
NM_022362, 3029
NM_022369, 4246
NM_022371, 527
NM_022442, 5806
NM_022453, 988
NM_022458, 2464
NM_022461, 1086
NM_022485, 1045
NM_022550, 1638
NM_022551, 1946
NM_022552, 717
NM_022566, 4296
NM_022727, 5961
NM_022744, 4468
NM_022747, 4084
NM_022748, 2226
NM_022752, 5474
NM_022758, 1926
NM_022770, 4539
NM_022778, 107
NM_022839, 4290
NM_022963, 1838
NM_023009, 152
NM_023011, 3940
NM_023032, 3691
NM_023033, 3693
NM_023078, 2620
NM_023936, 4378
NM_023942, 2449
NM_024003, 6336
NM_024026, 3872
NM_024027, 645
NM_024029, 5250
NM_024031, 4458
NM_024033, 2427
NM_024040, 3047
NM_024045, 2957
NM_024048, 4470
NM_024067, 2186
NM_024068, 3643
NM_024070, 2335
NM_024089, 3935
NM_024098, 3218
NM_024099, 3236
NM_024104, 5323
NM_024111, 4148
NM_024294, 1924
NM_024297, 4672
NM_024299, 5865
NM_024319, 614
NM_024321, 5389
NM_024329, 62
NM_024330, 379
NM_024333, 5186
NM_024339, 4396
NM_024407, 5120
NM_024507, 4406
NM_024516, 4502
NM_024537, 3938
NM_024567, 2508
NM_024571, 4350
NM_024572, 719
NM_024586, 247
NM_024589, 4346
NM_024602, 206
NM_024603, 241
NM_024613, 2584
NM_024627, 5951
NM_024640, 137
NM_024653, 2373
NM_024658, 3960
NM_024664, 183
NM_024668, 1724
NM_024671, 4454
NM_024691, 5636
NM_024709, 603
NM_024748, 1526
NM_024824, 4057
NM_024844, 4955
NM_024854, 3529
NM_024855, 5769
NM_024863, 6248
NM_024881, 5321
NM_024900, 1491
NM_024918, 5757
NM_024942, 3095
NM_025070, 2541
NM_025072, 2772
NM_025108, 4411
NM_025129, 5534
NM_025150, 358
NM_025164, 3374
NM_025168, 1863
NM_025197, 4830
NM_025202, 1000
NM_025203, 678
NM_025204, 6109
NM_025205, 1414
NM_025207, 455
NM_025226, 499
NM_025232, 2503
NM_025233, 4859
NM_025234, 4270
NM_025241, 5190
NM_025263, 2007
NM_030567, 1826
NM_030573, 5965
NM_030579, 4553
NM_030587, 196
NM_030593, 5411
NM_030775, 3432
NM_030782, 1545
NM_030815, 5719
NM_030819, 4573
NM_030877, 5763
NM_030900, 2232
NM_030920, 332
NM_030921, 1272
NM_030925, 3910
NM_030926, 1009
NM_030935, 2331
NM_030973, 5532
NM_031157, 3612
NM_031206, 6210
NM_031213, 5138
NM_031228, 5642
NM_031229, 5640
NM_031243, 2212
NM_031263, 2708
NM_031289, 3496
NM_031300, 1832
NM_031417, 5506
NM_031434, 2456
NM_031443, 2234
NM_031453, 2902
NM_031459, 131
NM_031465, 3446
NM_031472, 3261
NM_031478, 4522
NM_031479, 3665
NM_031482, 1629
NM_031484, 3070
NM_031485, 5574
NM_031901, 336
NM_031925, 2304
NM_031942, 905
NM_031966, 1598
NM_031968, 5014
NM_031989, 3622
NM_031990, 5100
NM_031992, 2290
NM_032023, 2923
NM_032038, 4495
NM_032088, 1770
NM_032092, 1772
NM_032112, 3031
NM_032140, 4571
NM_032162, 4310
NM_032164, 2340
NM_032196, 4150
NM_032204, 5996
NM_032207, 5317
NM_032211, 3068
NM_032212, 843
NM_032219, 1370
NM_032227, 6257
NM_032271, 4388
NM_032280, 1642
NM_032288, 1354
NM_032292, 412
NM_032299, 3395
NM_032313, 1437
NM_032322, 4771
NM_032323, 402
NM_032324, 630
NM_032330, 4485
NM_032331, 1318
NM_032333, 2996
NM_032338, 3712
NM_032342, 2746
NM_032343, 1235
NM_032350, 2163
NM_032361, 1814
NM_032376, 4854
NM_032377, 5262
NM_032379, 3346
NM_032383, 1280
NM_032390, 875
NM_032402, 1776
NM_032403, 1774
NM_032486, 4444
NM_032527, 5869
NM_032565, 3914
NM_032626, 4440
NM_032627, 5345
NM_032635, 5393
NM_032636, 296
NM_032637, 1577
NM_032642, 3434
NM_032656, 3851
NM_032667, 3240
NM_032712, 5588
NM_032726, 990
NM_032737, 5157
NM_032738, 503
NM_032747, 3061
NM_032750, 1174
NM_032753, 5173
NM_032756, 222
NM_032792, 5631
NM_032799, 2763
NM_032814, 3812
NM_032822, 785
NM_032827, 810
NM_032864, 245
NM_032871, 3326
NM_032872, 122
NM_032873, 3415
NM_032890, 606
NM_032904, 3794
NM_032905, 2893
NM_032907, 4248
NM_032928, 2860
NM_032929, 2081
NM_032933, 5037
NM_032951, 2284
NM_032953, 2286
NM_032958, 2376
NM_032989, 3258
NM_032997, 2949
NM_032999, 2295
NM_033008, 1176
NM_033010, 1178
NM_033011, 2538
NM_033022, 2978
NM_033046, 796
NM_033070, 5937
NM_033161, 2828
NM_033197, 5729
NM_033219, 2730
NM_033251, 4635
NM_033296, 1404
NM_033301, 2635
NM_033316, 1348
NM_033363, 5417
NM_033410, 4456
NM_033415, 5355
NM_033416, 878
NM_033421, 5787
NM_033440, 60
NM_033534, 15
NM_033544, 4315
NM_033551, 1785
NM_052837, 426
NM_052848, 5451
NM_052859, 1157
NM_052862, 488
NM_052881, 5656
NM_052886, 2602
NM_052936, 6251
NM_052963, 2616
NM_052984, 3685
NM_053043, 2462
NM_053056, 3311
NM_053275, 3829
NM_054012, 2822
NM_054013, 1848
NM_054014, 5650
NM_054016, 95
NM_057089, 2363
NM_057161, 1890
NM_057169, 3790
NM_057174, 3188
NM_057182, 5376
NM_058164, 5230
NM_058179, 2703
NM_058192, 4366
NM_058193, 3422
NM_058195, 2653
NM_058196, 2657
NM_058199, 2836
NM_078467, 1912
NM_079423, 3648
NM_079425, 3650
NM_080424, 1016
NM_080425, 5828
NM_080426, 5832
NM_080491, 3342
NM_080592, 696
NM_080594, 4394
NM_080598, 1984
NM_080648, 3999
NM_080649, 4001
NM_080670, 1726
NM_080686, 1981
NM_080687, 3942
NM_080702, 1977
NM_080703, 1975
NM_080796, 5855
NM_080797, 5859
NM_080820, 5693
NM_080822, 4654
NM_106552, 670
NM_130398, 639
NM_130442, 2260
NM_130468, 4143
NM_130898, 434
NM_133330, 1376
NM_133332, 1380
NM_133373, 4885
NM_133375, 4222
NM_133436, 2357
NM_133480, 1051
NM_133481, 1053
NM_133483, 3676
NM_133503, 3742
NM_133504, 3744
NM_133505, 3746
NM_133506, 3750
NM_133507, 3748
NM_133627, 4786
NM_133629, 4790
NM_133630, 4788
NM_133637, 798
NM_133645, 2066
NM_134269, 6009
NM_134323, 3616
NM_134324, 3618
NM_134440, 5358
NM_138385, 1372
NM_138391, 545
NM_138427, 4739
NM_138434, 2451
NM_138443, 5060
NM_238483, 1037
NM_138578, 5713
NM_138614, 1125
NM_138699, 1406
NM_138801, 727
NM_138924, 5124
XM_001289, 524
XM_001299, 33
XM_001389, 1453
XM_001468, 342
XM_001472, 250
XM_001482, 3658
XM_001589, 24
XM_001616, 101
XM_001640, 126
XM_001807, 135
XM_001812, 134
XM_001826, 78
XM_001897, 486
XM_001914, 567
XM_001916, 568
XM_001958, 599
XM_002068, 523
XM_002105, 141
XM_002114, 113
XM_002217, 845
XM_002255, 1361
XM_002435, 700
XM_002447, 877
XM_002480, 680
XM_002540, 1006
XM_002611, 823
XM_002636, 964
XM_002647, 770
XM_002669, 946
XM_002674, 776
XM_002704, 853
XM_002727, 788
XM_002739, 779
XM_002742, 1036
XM_002828, 1143
XM_002854, 1187
XM_002855, 1186
XM_002859, 1274
XM_002899, 1127
XM_003213, 1162
XM_003222, 1119
XM_003245, 1136
XM_003305, 1451
XM_003435, 1432
XM_003477, 1530
XM_003511, 1448
XM_003555, 1500
XM_003611, 2083
XM_003716, 1811
XM_003771, 1644
XM_003789, 1712
XM_003825, 1540
XM_003830, 1666
XM_003841, 1699
XM_003869, 1572
XM_003896, 1581
XM_003937, 1710
XM_004009, 1565
XM_004098, 3704
XM_004151, 2065
XM_004256, 2114
XM_004297, 2113
XM_004330, 3194
XM_004379, 2122
XM_004383, 2130
XM_004526, 2110
XM_004627, 2402
XM_004901, 2292
XM_005060, 2605
XM_005086, 1042
XM_005100, 2908
XM_005180, 1332
XM_005305, 2485
XM_005348, 2755
XM_005365, 2760
XM_005490, 2707
XM_005525, 2727
XM_005543, 2666
XM_005675, 3103
XM_005698, 3053
XM_005724, 2878
XM_005938, 3058
XM_005969, 3088
XM_006139, 3127
XM_006170, 3201
XM_006212, 3167
XM_006290, 98
XM_006297, 3196
XM_006424, 3151
XM_006432, 3371
XM_006464, 3355
XM_006467, 3399
XM_006475, 3135
XM_006483, 3136
XM_006529, 3281
XM_006533, 3270
XM_006566, 3849
XM_006578, 3736
XM_006589, 3766
XM_006595, 3835
XM_006694, 3535
XM_006710, 3626
XM_006748, 3536
XM_006826, 3559
XM_006887, 3765
XM_006925, 3485
XM_006936, 3483
XM_006937, 5074
XM_006947, 3482
XM_006958, 3475
XM_007002, 3797
XM_007003, 3796
XM_007199, 3923
XM_007254, 4097
XM_007272, 4081
XM_007288, 3968
XM_007293, 3967
XM_007315, 3958
XM_007316, 3957
XM_007324, 4027
XM_007328, 4024
XM_007441, 4045
XM_007483, 4072
XM_007488, 4005
XM_007491, 3996
XM_007531, 4167
XM_007545, 4156
XM_007623, 4221
XM_007651, 4189
XM_007751, 4129
XM_007963, 4474
XM_007988, 4430
XM_008064, 4509
XM_008065, 4497
XM_008106, 4463
XM_008126, 4353
XM_008150, 4800
XM_008231, 4694
XM_008253, 4926
XM_008323, 4750
XM_008334, 4671
XM_008351, 4856
XM_008401, 4867
XM_008402, 4869
XM_008432, 4902
XM_008441, 4686
XM_008459, 4915
XM_008462, 4777
XM_008486, 4760
XM_008509, 4658
XM_008538, 4684
XM_008557, 4650
XM_008579, 4809
XM_008679, 4693
XM_008695, 5089
XM_008723, 5054
XM_008812, 5083
XM_008830, 5597
XM_008851, 5522
XM_008854, 5325
XM_008860, 5485
XM_008878, 5472
XM_008887, 5243
XM_008912, 5453
XM_008985, 5531
XM_009010, 5205
XM_009036, 5486
XM_009063, 5274
XM_009082, 5256
XM_009125, 5484
XM_009126, 5496
XM_009149, 5406
XM_009180, 5378
XM_009203, 5443
XM_009222, 5165
XM_009277, 5113
XM_009279, 5110
XM_009293, 5338
XM_009303, 5310
XM_009330, 5357
XM_009338, 5384
XM_009436, 5705
XM_009450, 5728
XM_009501, 5754
XM_009549, 5816
XM_009622, 5647
XM_009642, 5759
XM_009671, 5823
XM_009672, 5821
XM_009686, 5762
XM_009805, 5919
XM_009947, 6022
XM_009967, 6031
XM_009973, 6042
XM_010000, 6063
XM_010002, 6064
XM_010024, 6087
XM_010029, 6094
XM_010040, 6103
XM_010055, 6108
XM_010117, 6269
XM_010141, 6216
XM_010156, 5266
XM_010178, 6310
XM_010272, 6132
XM_010362, 6274
XM_010378, 6169
XM_010436, 6280
XM_010494, 3429
XM_010615, 253
XM_010636, 451
XM_010664, 133
XM_010682, 581
XM_010712, 182
XM_010732, 593
XM_010778, 925
XM_010852, 938
XM_010858, 1004
XM_010866, 992
XM_010881, 771
XM_010886, 755
XM_010938, 4641
XM_010941, 1433
XM_010953, 1130
XM_010978, 1290
XM_011074, 1320
XM_011089, 5076
XM_011117, 2059
XM_011118, 4941
XM_011129, 1423
XM_011160, 1365
XM_011548, 2411
XM_011618, 2400
XM_011629, 2533
XM_011642, 2586
XM_011650, 66
XM_011657, 2592
XM_011749, 2798
XM_011752, 2786
XM_011769, 2562
XM_011778, 2832
XM_011988, 3260
XM_012124, 3836
XM_012145, 3761
XM_012159, 3494
XM_012162, 3598
XM_012179, 5337
XM_012182, 3638
XM_012184, 3861
XM_012219, 3759
XM_012272, 3543
XM_012284, 2395
XM_012376, 3990
XM_012377, 3983
XM_012398, 4133
XM_012418, 4199
XM_012462, 4322
XM_012487, 4555
XM_012549, 4734
XM_012569, 4461
XM_012609, 4945
XM_012615, 4744
XM_012634, 4950
XM_012638, 3874
XM_012642, 4849
XM_012651, 4916
XM_012676, 4675
XM_012741, 5031
XM_012798, 5212
XM_012812, 5370
XM_012860, 5439
XM_012862, 5195
XM_012913, 5114
XM_012931, 5768
XM_012970, 5700
XM_013010, 6066
XM_013015, 6089
XM_013029, 6118
XM_013042, 6207
XM_013060, 6196
XM_013086, 6145
XM_013112, 2530
XM_013127, 2577
XM_015234, 75
XM_015241, 5088
XM_015243, 3148
XM_015258, 2244
XM_015366, 4239
XM_015434, 547
XM_015462, 1208
XM_015468, 3596
XM_015476, 3585
XM_015481, 3580
XM_015516, 6206
XM_015563, 1525
XM_015652, 2937
XM_015697, 5264
XM_015700, 4478
XM_015705, 3214
XM_015717, 257
XM_015755, 5046
XM_015769, 5369
XM_015835, 4311
XM_015840, 3921
XM_015842, 3932
XM_015920, 909
XM_015922, 911
XM_016047, 2604
XM_016076, 4237
XM_016093, 2992
XM_016113, 2712
XM_016125, 6275
XM_016139, 3170
XM_016164, 276
XM_016170, 1554
XM_016199, 600
XM_016288, 880
XM_016308, 2726
XM_016334, 1294
XM_016345, 1799
XM_016351, 3924
XM_016378, 5364
XM_016382, 5036
XM_016410, 5438
XM_016480, 326
XM_016486, 4071
XM_016487, 4068
XM_016605, 3708
XM_016625, 773
XM_016640, 3538
XM_016674, 1652
XM_016700, 2433
XM_016713, 4165
XM_016733, 2256
XM_016843, 766
XM_016857, 1941
XM_016871, 5180
XM_016985, 4213
XM_017080, 3436
XM_017096, 4644
XM_017204, 5240
XM_017234, 4712
XM_017240, 4135
XM_017315, 67
XM_017356, 1291
XM_017364, 1105
XM_017369, 3394
XM_017432, 3895
XM_017442, 2313
XM_017474, 1679
XM_017483, 2280
XM_017508, 3710
XM_017517, 2080
XM_017578, 4980
XM_017591, 1701
XM_017641, 1544
XM_017698, 861
XM_017816, 2581
XM_017831, 2119
XM_017846, 109
XM_017857, 1640
XM_017914, 3953
XM_017925, 1476
XM_017930, 6284
XM_017931, 2659
XM_017971, 4319
XM_017984, 4338
XM_017996, 2711
XM_018006, 2710
XM_018019, 6157
XM_018039, 784
XM_018041, 642
XM_018054, 4123
XM_018088, 4472
XM_018108, 6313
XM_018109, 6315
XM_018136, 161
XM_018142, 6232
XM_018149, 1264
XM_018167, 3015
XM_018182, 2098
XM_018205, 64
XM_018241, 6161
XM_018279, 3057
XM_018287, 2595
XM_018301, 763
XM_018332, 314
XM_018359, 2281
XM_018399, 3918
XM_018432, 4331
XM_018473, 1658
XM_018515, 5354
XM_018523, 1359
XM_018534, 4840
XM_018539, 6014
XM_018540, 841
XM_026944, 2787
XM_026951, 2771
XM_026968, 2769
XM_026985, 2766
XM_026987, 2765
XM_027102, 3802
XM_027143, 6106
XM_027161, 1220
XM_027214, 2385
XM_027309, 4329
XM_027313, 226
XM_027365, 4334
XM_027412, 4368
XM_027440, 2505
XM_027558, 4352
XM_027651, 2490
XM_027679, 2488
XM_027825, 4661
XM_027904, 5548
XM_027916, 76
XM_027952, 6353
XM_027963, 936
XM_027964, 1619
XM_027983, 213
XM_028034, 940
XM_028064, 5119
XM_028067, 5117
XM_028151, 4562
XM_028192, 3117
XM_028263, 5488
XM_028267, 5491
XM_028322, 4075
XM_028347, 4074
XM_028358, 4073
XM_028398, 4667
XM_028417, 4678
XM_028643, 3624
XM_028662, 3561
XM_028666, 5383
XM_028672, 5382
XM_028744, 5025
XM_028760, 3554
XM_028783, 5851
XM_028806, 5765
XM_028810, 5766
XM_028834, 5863
XM_028848, 4390
XM_028918, 5867
XM_028966, 5871
XM_029031, 169
XM_029096, 1539
XM_029104, 1314
XM_029132, 1313
XM_029136, 1310
XM_029168, 2841
XM_029187, 6194
XM_029228, 2069
XM_029288, 4067
XM_029369, 1198
XM_029438, 4656
XM_029450, 5404
XM_029455, 5403
XM_029461, 6282
XM_029567, 2609
XM_029631, 3602
XM_029728, 3595
XM_029746, 2128
XM_029805, 3507
XM_029810, 5776
XM_029822, 5778
XM_029842, 176
XM_029844, 145
XM_030044, 5796
XM_030203, 1028
XM_030268, 2543
XM_030274, 2544
XM_030326, 3187
XM_030373, 6233
XM_030417, 1112
XM_030423, 154
XM_030447, 3065
XM_030470, 68
XM_030485, 5159
XM_030529, 862
XM_030582, 883
XM_030621, 5818
XM_030699, 5834
XM_030714, 5145
XM_030720, 5137
XM_030721, 5135
XM_030771, 1821
XM_030777, 1823
XM_030782, 1824
XM_030812, 1256
XM_030834, 952
XM_030895, 5465
XM_030901, 5456
XM_030914, 5450
XM_030920, 40
XM_031025, 4032
XM_031074, 4039
XM_031251, 5307
XM_031263, 5305
XM_031273, 5303
XM_031276, 5302
XM_031292, 4295
XM_031320, 1445
XM_031345, 5292
XM_031354, 4292
XM_031404, 4285
XM_031415, 4767
XM_031427, 4769
XM_031466, 4765
XM_031515, 4147
XM_031519, 731
XM_031527, 733
XM_031536, 4758
XM_031554, 4145
XM_031585, 782
XM_031586, 783
XM_031596, 780
XM_031617, 4138
XM_031626, 738
XM_031718, 4159
XM_031807, 3491
XM_031857, 5184
XM_031866, 3041
XM_031890, 3044
XM_031917, 5176
XM_031944, 5066
XM_031949, 3049
XM_031992, 3059
XM_032020, 5281
XM_032121, 2455
XM_032201, 4836
XM_032216, 2454
XM_032269, 1221
XM_032285, 5399
XM_032391, 216
XM_032403, 4180
XM_032443, 3930
XM_032476, 2976
XM_032520, 2970
XM_032553, 1626
XM_032588, 3457
XM_032614, 3462
XM_032710, 5247
XM_032719, 5248
XM_032724, 5252
XM_032759, 1700
XM_032766, 4864
XM_032774, 5257
XM_032782, 5261
XM_032813, 4863
XM_032817, 4861
XM_032852, 4857
XM_032895, 1590
XM_032902, 1588
XM_032930, 6189
XM_032944, 2470
XM_032996, 5943
XM_033015, 5902
XM_033016, 5903
XM_033090, 5946
XM_033147, 6241
XM_033227, 3450
XM_033232, 6351
XM_033251, 3959
XM_033263, 3472
XM_033294, 1123
XM_033337, 3964
XM_033355, 2819
XM_033359, 2818
XM_033360, 2817
XM_033361, 2815
XM_033362, 2811
XM_033380, 2809
XM_033385, 2808
XM_033391, 3969
XM_033424, 2774
XM_033435, 3975
XM_033445, 3980
XM_033457, 2777
XM_033460, 2778
XM_033553, 3991
XM_033595, 3994
XM_033654, 79
XM_033683, 77
XM_033689, 4646
XM_033714, 4645
XM_033813, 5960
XM_033862, 6173
XM_033876, 2383
XM_033878, 6172
XM_033884, 6170
XM_033910, 2134
XM_033912, 2132
XM_033922, 4606
XM_034000, 501
XM_034082, 454
XM_034321, 1502
XM_034375, 4460
XM_034377, 5623
XM_034431, 3185
XM_034586, 4376
XM_034590, 4380
XM_034640, 2638
XM_034662, 319
XM_034671, 318
XM_034710, 1466
XM_034713, 1468
XM_034744, 1655
XM_034862, 1675
XM_034890, 4184
XM_034897, 4256
XM_034935, 6201
XM_034952, 857
XM_034953, 4116
XM_035014, 4119
XM_035103, 2824
XM_035107, 2439
XM_035109, 2825
XM_035220, 800
XM_035368, 2626
XM_035370, 2631
XM_035373, 2629
XM_035465, 6123
XM_035485, 3571
XM_035490, 3564
XM_035497, 3562
XM_035572, 1392
XM_035625, 5197
XM_035627, 5196
XM_035636, 5194
XM_035638, 5192
XM_035640, 5034
XM_035662, 2483
XM_035680, 2482
XM_035824, 1402
XM_035919, 5612
XM_035986, 1456
XM_035999, 5907
XM_036002, 1440
XM_036011, 5910
XM_036042, 5913
XM_036087, 5917
XM_036104, 4965
XM_036107, 5923
XM_036115, 4971
XM_036118, 1262
XM_036175, 5924
XM_036299, 155
XM_036339, 3178
XM_036413, 2469
XM_036450, 664
XM_036462, 4827
XM_036465, 4825
XM_036500, 573
XM_036507, 575
XM_036528, 4410
XM_036556, 566
XM_036593, 2939
XM_036659, 4707
XM_036680, 4342
XM_036727, 4134
XM_036744, 433
XM_036755, 5927
XM_036785, 4982
XM_036829, 442
XM_036845, 450
XM_036934, 448
XM_036937, 5969
XM_036938, 1197
XM_037002, 1668
XM_037056, 2107
XM_037101, 873
XM_037108, 831
XM_037147, 3212
XM_037173, 3202
XM_037195, 4988
XM_037196, 4987
XM_037202, 5840
XM_037206, 5842
XM_037217, 5846
XM_037260, 1608
XM_037329, 591
XM_037377, 1300
XM_037381, 1299
XM_037423, 1163
XM_037468, 6114
XM_037474, 6116
XM_037565, 5106
XM_037572, 5109
XM_037600, 1304
XM_037657, 2608
XM_037662, 5372
XM_037682, 5977
XM_037741, 2276
XM_037778, 4244
XM_037797, 5981
XM_037808, 3263
XM_037875, 2045
XM_037945, 5993
XM_037971, 4897
XM_038030, 2855
XM_038049, 2864
XM_038063, 2866
XM_038098, 5343
XM_038146, 5339
XM_038221, 1695
XM_038243, 1341
XM_038308, 3737
XM_038371, 3902
XM_038391, 2757
XM_038424, 5018
XM_038536, 2909
XM_038576, 734
XM_038584, 6019
XM_038659, 3533
XM_038791, 3841
XM_038852, 244
XM_038872, 5062
XM_038911, 237
XM_038946, 1840
XM_039165, 1413
XM_039173, 1416
XM_039176, 1417
XM_039225, 4125
XM_039236, 6047
XM_039248, 6051
XM_039306, 4551
XM_039339, 6060
XM_039372, 6065
XM_039395, 3732
XM_039474, 4794
XM_039654, 2646
XM_039702, 4200
XM_039712, 716
XM_039721, 321
XM_039723, 5140
XM_039796, 1292
XM_039805, 1258
XM_039908, 5598
XM_039910, 4721
XM_039921, 4732
XM_039952, 1213
XM_039975, 1783
XM_040009, 377
XM_040066, 6088
XM_040095, 6091
XM_040221, 3707
XM_040267, 2879
XM_040272, 2876
XM_040321, 1524
XM_040498, 2417
XM_040623, 2074
XM_040644, 3734
XM_040709, 315
XM_040752, 1493
XM_040853, 2218
XM_040898, 4100
XM_040942, 4094
XM_040952, 4090
XM_041014, 4086
XM_041020, 2697
XM_041059, 1670
XM_041100, 3503
XM_041209, 3925
XM_041211, 1161
XM_041221, 1410
XM_041235, 4008
XM_041248, 6111
XM_041473, 3928
XM_041484, 3944
XM_041507, 1147
XM_041583, 4957
XM_041678, 5027
XM_041694, 1614
XM_041712, 1592
XM_041872, 5090
XM_041879, 353
XM_041884, 354
XM_041921, 6304
XM_041964, 4680
XM_042018, 5095
XM_042025, 1600
XM_042153, 6348
XM_042155, 6346
XM_042168, 1286
XM_042301, 1474
XM_042326, 1032
XM_042422, 2145
XM_042473, 2148
XM_042618, 1229
XM_042621, 4596
XM_042658, 2561
XM_042695, 1364
XM_042698, 4710
XM_042765, 5701
XM_042781, 2434
XM_042788, 2744
XM_042841, 1072
XM_042852, 3339
XM_042860, 1070
XM_042963, 6295
XM_042967, 537
XM_042968, 6297
XM_043047, 4577
XM_043173, 866
XM_043220, 3111
XM_043340, 1805
XM_043388, 1808
XM_043589, 2998
XM_043605, 2999
XM_043614, 6099
XM_043643, 6250
XM_043771, 1568
XM_044075, 416
XM_044077, 391
XM_044127, 398
XM_044128, 408
XM_044166, 406
XM_044172, 411
XM_044334, 3859
XM_044354, 2968
XM_044367, 4938
XM_044372, 4943
XM_044376, 4935
XM_044394, 4927
XM_044426, 4924
XM_044523, 4304
XM_044533, 4307
XM_044565, 4269
XM_044569, 4272
XM_044593, 4278
XM_044608, 5213
XM_044619, 5210
XM_044627, 2563
XM_044866, 2139
XM_044914, 5658
XM_044915, 5660
XM_044932, 3129
XM_044957, 3131
XM_045010, 3821
XM_045044, 4749
XM_045104, 4989
XM_045140, 2973
XM_045151, 5226
XM_045170, 928
XM_045183, 4651
XM_045187, 3833
XM_045283, 757
XM_045290, 1214
XM_045296, 2759
XM_045401, 2403
XM_045418, 5667
XM_045451, 5671
XM_045460, 5674
XM_045499, 3276
XM_045525, 3278
XM_045535, 4751
XM_045551, 4752
XM_045581, 4996
XM_045602, 3856
XM_045612, 3273
XM_045613, 3271
XM_045642, 3269
XM_045667, 3074
XM_045681, 4287
XM_045750, 3157
XM_045802, 3826
XM_045856, 2407
XM_045901, 4852
XM_045952, 2413
XM_045963, 3834
XM_046001, 2414
XM_046035, 4453
XM_046041, 3726
XM_046057, 1443
XM_046090, 5423
XM_046160, 5708
XM_046179, 5710
XM_046313, 5544
XM_046349, 187
XM_046401, 1085
XM_046419, 5578
XM_046450, 201
XM_046464, 522
XM_046472, 5004
XM_046481, 4999
XM_046520, 5689
XM_046551, 212
XM_046557, 208
XM_046565, 204
XM_046642, 3951
XM_046648, 3950
XM_046651, 3949
XM_046743, 3035
XM_046765, 5020
XM_046767, 5022
XM_046769, 5021
XM_046822, 5150
XM_046836, 2722
XM_046863, 2720
XM_046918, 112
XM_046932, 4958
XM_046934, 5160
XM_047007, 5723
XM_047011, 5725
XM_047018, 5727
XM_047024, 6177
XM_047032, 6176
XM_047083, 2521
XM_047175, 690
XM_047374, 5446
XM_047376, 5445
XM_047409, 5444
XM_047436, 4624
XM_047477, 1429
XM_047479, 495
XM_047499, 610
XM_047525, 4632
XM_047545, 616
XM_047561, 1137
XM_047584, 5131
XM_047600, 5132
XM_047964, 1798
XM_048088, 753
XM_048119, 4344
XM_048258, 5385
XM_048286, 3255
XM_048351, 5218
XM_048364, 5219
XM_048404, 6329
XM_048410, 6328
XM_048420, 6325
XM_048471, 5082
XM_048479, 2679
XM_048518, 2684
XM_048539, 2686
XM_048603, 3674
XM_048654, 4829
XM_048690, 1007
XM_048780, 57
XM_048859, 2881
XM_048905, 6306
XM_048943, 3640
XM_048957, 3931
XM_048991, 3642
XM_049048, 3652
XM_049108, 820
XM_049113, 822
XM_049116, 818
XM_049141, 3586
XM_049148, 3581
XM_049150, 3659
XM_049197, 3161
XM_049201, 3772
XM_049211, 3771
XM_049226, 2623
XM_049237, 5391
XM_049247, 2618
XM_049282, 5223
XM_049310, 139
XM_049337, 6320
XM_049354, 4275
XM_049372, 4317
XM_049421, 2637
XM_049502, 5236
XM_049561, 5239
XM_049663, 3493
XM_049680, 476
XM_049690, 483
XM_049742, 14
XM_049795, 3082
XM_049899, 2121
XM_049904, 3937
XM_049920, 5482
XM_049931, 4995
XM_049934, 4994
XM_049937, 4818
XM_050074, 3528
XM_050101, 4773
XM_050159, 4880
XM_050194, 4462
XM_050200, 1487
XM_050215, 2525
XM_050236, 5602
XM_050265, 2278
XM_050278, 4103
XM_050293, 2487
XM_050403, 6192
XM_050430, 2389
XM_050435, 5227
XM_050506, 2583
XM_050534, 4348
XM_050552, 1234
XM_050589, 5603
XM_050638, 979
XM_050660, 5330
XM_050731, 2571
XM_050891, 984
XM_050962, 975
XM_050964, 4220
XM_051219, 4479
XM_051264, 1237
XM_051298, 2612
XM_051364, 5290
XM_051430, 3398
XM_051435, 3358
XM_051463, 4230
XM_051471, 6238
XM_051476, 6237
XM_051489, 3367
XM_051518, 1131
XM_051556, 6
XM_051586, 5092
XM_051712, 4025
XM_051716, 3373
XM_051763, 4727
XM_051778, 4600
XM_051860, 4298
XM_051877, 515
XM_052113, 3378
XM_052310, 1060
XM_052313, 1535
XM_052336, 1477
XM_052460, 3714
XM_052474, 3719
XM_052530, 1424
XM_052542, 3755
XM_052626, 1398
XM_052635, 5166
XM_052641, 3769
XM_052661, 5168
XM_052721, 2056
XM_052725, 2784
XM_052786, 3153
XM_052862, 3404
XM_052893, 3825
XM_052974, 608
XM_052989, 817
XM_053074, 5430
XM_053122, 1363
XM_053164, 3641
XM_053183, 58
XM_053206, 2875
XM_053245, 400
XM_053323, 1078
XM_053585, 4252
XM_053633, 544
XM_053712, 1074
XM_053717, 4663
XM_053787, 3283
XM_053796, 3288
XM_053952, 3722
XM_053955, 1859
XM_054038, 4832
XM_054098, 6183
XM_054221, 6155
XM_054344, 4973
XM_054474, 2933
XM_054475, 2935
XM_054520, 1047
XM_054566, 5926
XM_054706, 2146
XM_054752, 2849
XM_054763, 2852
XM_054856, 3193
XM_054868, 228
XM_054900, 4309
XM_054978, 295
XM_055013, 3853
XM_055061, 4826
XM_055132, 4514
XM_055195, 4427
XM_055199, 4942
XM_055230, 5336
XM_055254, 954
XM_055369, 3397
XM_055481, 251
XM_055551, 1461
XM_055573, 3086
XM_055641, 2064
XM_055658, 5592
XM_055686, 5163
XM_055771, 4505
XM_055859, 5483
XM_055880, 583
XM_055993, 5646
XM_056035, 5678
XM_056082, 4648
XM_056260, 4438
XM_056286, 5582
XM_056315, 1723
XM_056317, 4077
XM_056346, 3645
XM_056353, 3662
XM_056421, 5175
XM_056481, 3545
XM_056602, 5408
XM_056681, 3700
XM_056730, 4775
XM_056884, 618
XM_056923, 521
XM_056957, 1471
XM_056963, 1793
XM_056970, 628
XM_056996, 3798
XM_057020, 4257
XM_057074, 5260
XM_057150, 4619
XM_057236, 5756
XM_057374, 5793
XM_057492, 1548
XM_057664, 740
XM_057780, 2557
XM_057994, 1541
XM_058039, 1934
XM_058098, 986
XM_058116, 4526
XM_058125, 5635
XM_058210, 4018
XM_058232, 5225
XM_058240, 102
XM_058247, 466
XM_058266, 2144
XM_058267, 1278
XM_058343, 3020
XM_058361, 3078
XM_058405, 552
XM_058406, 3084
XM_058414, 3159
XM_058450, 3352
XM_058505, 3125
XM_058528, 3671
XM_058556, 3773
XM_058567, 3504
XM_058574, 3454
XM_058602, 3022
XM_058611, 3926
XM_058618, 4091
XM_058636, 4118
XM_058646, 3986
XM_058647, 3978
XM_058677, 4061
XM_058684, 4186
XM_058699, 4250
XM_058702, 294
XM_058739, 4621
XM_058745, 4543
XM_058784, 4404
XM_058796, 4337
XM_058830, 4803
XM_058867, 4755
XM_058900, 4730
XM_058918, 5949
XM_058927, 1441
XM_058949, 5463
XM_058967, 5295
XM_058968, 2619
XM_058977, 3920
XM_058987, 5570
XM_058990, 5584
XM_058991, 5552
XM_059045, 5419
XM_059052, 5447
XM_059066, 114
XM_059067, 120
XM_059088, 130
XM_059094, 465
XM_059117, 103
XM_059120, 562
XM_059133, 224
XM_059171, 171
XM_059180, 256
XM_059191, 492
XM_059201, 1
XM_059210, 330
XM_059214, 185
XM_059230, 55
XM_059268, 5675
XM_059321, 5607
XM_059335, 6013
XM_059351, 920
XM_059368, 653
XM_059372, 1029
XM_059422, 968
XM_059461, 971
XM_059465, 907
XM_059516, 1266
XM_059557, 1068
XM_059561, 1059
XM_059583, 1252
XM_059593, 1434
XM_059623, 1519
XM_059628, 1442
XM_059633, 1469
XM_059637, 2804
XM_059653, 1596
XM_059669, 1617
XM_059709, 1604
XM_059720, 2914
XM_059741, 2118
XM_059745, 2131
XM_059773, 2141
XM_059776, 2062
XM_059801, 1939
XM_059839, 2430
XM_059876, 2282
XM_059933, 2531
XM_059945, 2838
XM_059961, 2859
XM_059966, 2871
XM_059979, 2644
XM_059986, 2813
XM_059998, 2673
XM_060006, 2647
XM_060012, 4115
XM_060030, 6146
XM_060042, 4281
XM_060067, 1499
XM_060331, 509
XM_060517, 531
XM_060976, 2885
XM_061125, 2931
XM_061126, 2930
XM_062437, 3775
XM_063639, 4234
XM_064091, 4597
XM_065884, 777
XM_066291, 5998
XM_066900, 6261
XM_067264, 1240
XM_067325, 5030
XM_067715, 1169
XM_068164, 1497
XM_068395, 1789
XM_068853, 1714
XM_068919, 2085
XM_068963, 2072
XM_070188, 2480
XM_070203, 2473
XM_070873, 2742
XM_071178, 2705
XM_071580, 1557
XM_071605, 2381
XM_071623, 1439
XM_071801, 4122
XM_071873, 4630
XM_071937, 2152
XM_072173, 5876
XM_072430, 2387
XM_072526, 2857
XM_076414, 1199
XM_083842, 3026
XM_083852, 3141
XM_083864, 3774
XM_083866, 3715
XM_083868, 3590
XM_083892, 3787
XM_083939, 4364
XM_083966, 4923
XM_083983, 4881
XM_084007, 5055
XM_084014, 5246
XM_084023, 5528
XM_084026, 5549
XM_084055, 580
XM_084084, 6090
XM_084110, 1340
XM_084111, 1243
XM_084120, 1315
XM_084123, 1263
XM_084129, 1231
XM_084141, 1041
XM_084158, 1465
XM_084168, 1547
XM_084179, 1591
XM_084180, 1781
XM_084204, 2079
XM_084238, 2453
XM_084241, 2337
XM_084270, 2851
XM_084283, 6229
XM_084287, 6203
XM_084288, 6153
XM_084296, 6227
XM_084311, 6350
XM_084359, 3073
XM_084372, 3016
XM_084385, 2944
XM_084413, 3028
XM_084420, 2910
XM_084429, 2911
XM_084450, 2942
XM_084451, 2953
XM_084467, 2994
XM_084477, 3010
XM_084480, 3012
XM_084505, 3080
XM_084514, 3180
XM_084515, 3183
XM_084516, 3182
XM_084517, 3184
XM_084522, 3424
XM_084525, 3428
XM_084527, 3169
XM_084570, 3357
XM_084601, 3353
XM_084610, 3350
XM_084632, 3072
XM_084645, 3731
XM_084654, 3388
XM_084658, 3382
XM_084681, 3195
XM_084702, 3287
XM_084739, 3124
XM_084742, 3122
XM_084770, 3515
XM_084789, 3599
XM_084800, 3783
XM_084801, 3672
XM_084807, 3531
XM_084808, 3818
XM_084824, 3630
XM_084841, 3540
XM_084866, 3557
XM_084884, 3583
XM_084885, 3582
XM_084889, 3814
XM_084901, 3488
XM_084909, 3702
XM_084912, 3705
XM_084918, 3500
XM_084922, 3495
XM_084941, 3788
XM_084946, 3800
XM_084948, 3804
XM_084982, 3870
XM_084997, 3933
XM_084998, 2142
XM_085017, 3893
XM_085044, 3916
XM_085065, 4044
XM_085066, 4033
XM_085068, 1480
XM_085106, 3987
XM_085125, 4031
XM_085127, 4014
XM_085141, 4019
XM_085151, 4050
XM_085162, 4054
XM_085166, 3955
XM_085203, 4130
XM_085204, 4132
XM_085215, 4282
XM_085239, 4254
XM_085249, 4236
XM_085262, 4314
XM_085280, 4289
XM_085283, 4211
XM_085307, 4160
XM_085327, 4622
XM_085340, 4448
XM_085393, 4480
XM_085395, 4482
XM_085408, 4637
XM_085434, 4524
XM_085442, 4513
XM_085445, 4425
XM_085452, 4435
XM_085471, 4558
XM_085475, 4561
XM_085483, 4616
XM_085525, 4323
XM_085531, 4977
XM_085545, 4741
XM_085548, 4735
XM_085563, 4991
XM_085581, 472
XM_085589, 4948
XM_085613, 4724
XM_085627, 4951
XM_085636, 4873
XM_085672, 4757
XM_085687, 4659
XM_085691, 4677
XM_085716, 4992
XM_085722, 4745
XM_085735, 5019
XM_085743, 4718
XM_085775, 5058
XM_085779, 5075
XM_085788, 5049
XM_085789, 5043
XM_085790, 5045
XM_085791, 5042
XM_085856, 5501
XM_085862, 5244
XM_085874, 5460
XM_085875, 5461
XM_085876, 5462
XM_085909, 5297
XM_085916, 5285
XM_085917, 5276
XM_085927, 5527
XM_085928, 5489
XM_085934, 5537
XM_085935, 5573
XM_085950, 5487
XM_085971, 5371
XM_085972, 5629
XM_085981, 4599
XM_085986, 5398
XM_086004, 5425
XM_086074, 5311
XM_086101, 5128
XM_086102, 5130
XM_086116, 5331
XM_086132, 304
XM_086138, 282
XM_086142, 557
XM_086151, 46
XM_086164, 277
XM_086165, 279
XM_086166, 281
XM_086167, 280
XM_086178, 4
XM_086180, 19
XM_086204, 38
XM_086228, 1356
XM_086244, 601
XM_086245, 602
XM_086257, 632
XM_086271, 383
XM_086278, 4434
XM_086282, 543
XM_086296, 331
XM_086324, 214
XM_086328, 542
XM_086343, 265
XM_086357, 85
XM_086360, 29
XM_086375, 97
XM_086378, 485
XM_086381, 479
XM_086384, 178
XM_086389, 243
XM_086391, 231
XM_086397, 323
XM_086400, 366
XM_086428, 2161
XM_086431, 589
XM_086432, 592
XM_086444, 136
XM_086481, 490
XM_086484, 494
XM_086485, 493
XM_086494, 538
XM_086515, 324
XM_086518, 317
XM_086543, 190
XM_086552, 432
XM_086564, 388
XM_086567, 430
XM_086586, 52
XM_086587, 54
XM_086648, 5819
XM_086701, 5687
XM_086710, 5670
XM_086715, 5695
XM_086736, 5717
XM_086745, 5712
XM_086759, 5877
XM_086760, 5878
XM_086770, 5914
XM_086773, 5928
XM_086777, 5930
XM_086779, 5064
XM_086805, 5963
XM_086809, 5953
XM_086821, 5985
XM_086830, 6043
XM_086844, 6074
XM_086873, 5964
XM_086875, 6093
XM_086920, 805
XM_086923, 849
XM_086925, 850
XM_086944, 933
XM_086950, 858
XM_086961, 926
XM_086980, 791
XM_087028, 942
XM_087038, 2803
XM_087040, 842
XM_087041, 2800
XM_087045, 932
XM_087051, 748
XM_087061, 912
XM_087062, 914
XM_087068, 775
XM_087069, 772
XM_087118, 891
XM_087122, 839
XM_087151, 683
XM_087162, 985
XM_087166, 993
XM_087181, 965
XM_087193, 726
XM_087195, 725
XM_087206, 669
XM_087211, 743
XM_087218, 1011
XM_087240, 901
XM_087254, 1302
XM_087268, 1203
XM_087278, 1358
XM_087284, 1075
XM_087289, 1323
XM_087295, 1322
XM_087297, 1360
XM_087322, 1312
XM_087331, 1211
XM_087341, 1267
XM_087342, 1265
XM_087346, 1115
XM_087349, 1106
XM_087359, 1343
XM_087370, 1101
XM_087392, 1333
XM_087410, 1347
XM_087448, 1184
XM_087480, 3000
XM_087498, 1463
XM_087514, 1483
XM_087527, 1455
XM_087583, 1418
XM_087588, 1120
XM_087597, 1549
XM_087599, 1551
XM_087600, 1553
XM_087601, 1550
XM_087610, 1597
XM_087611, 1595
XM_087614, 1564
XM_087621, 1711
XM_087635, 1660
XM_087637, 1662
XM_087652, 1713
XM_087659, 1537
XM_087686, 1543
XM_087710, 3247
XM_087713, 1559
XM_087745, 1656
XM_087773, 1816
XM_087790, 1631
XM_087823, 1858
XM_087834, 2123
XM_087836, 2124
XM_087853, 2090
XM_087855, 2089
XM_087939, 2000
XM_087945, 1990
XM_087955, 3857
XM_087960, 1883
XM_087990, 1936
XM_087991, 2154
XM_088009, 3106
XM_088020, 1621
XM_088073, 2386
XM_088099, 2416
XM_088103, 2418
XM_088105, 2409
XM_088107, 605
XM_088119, 2422
XM_088122, 2420
XM_088135, 2446
XM_088180, 2352
XM_088239, 2297
XM_088264, 2195
XM_088294, 2529
XM_088316, 2611
XM_088321, 2628
XM_088323, 2574
XM_088325, 2572
XM_088336, 2519
XM_088338, 2515
XM_088370, 2613
XM_088399, 2559
XM_088401, 2560
XM_088422, 2839
XM_088426, 2833
XM_088459, 2847
XM_088461, 2870
XM_088472, 1472
XM_088550, 2640
XM_088552, 2641
XM_088553, 2642
XM_088563, 2672
XM_088569, 2748
XM_088571, 2750
XM_088587, 4120
XM_088588, 4114
XM_088589, 4121
XM_088592, 6311
XM_088619, 6151
XM_088622, 6152
XM_088630, 6209
XM_088637, 2700
XM_088638, 768
XM_088665, 6158
XM_088688, 6220
XM_088689, 6218
XM_088710, 6253
XM_088736, 6265
XM_088738, 6267
XM_088739, 6268
XM_088745, 6289
XM_088747, 6128
XM_088788, 338
XM_088863, 286
XM_088945, 507
XM_089030, 622
XM_089138, 254
XM_089514, 3019
XM_089551, 3006
XM_090218, 3542
XM_090413, 3779
XM_090458, 3767
XM_090833, 638
XM_090914, 4082
XM_090991, 4191
XM_091076, 1091
XM_091100, 4263
XM_091108, 4124
XM_091159, 4157
XM_091270, 4483
XM_091399, 4590
XM_091420, 4544
XM_091786, 3426
XM_091886, 5595
XM_091938, 5221
XM_091981, 5586
XM_091984, 5396
XM_092042, 5108
XM_092046, 5341
XM_092049, 5380
XM_092135, 672
XM_092158, 918
XM_092346, 944
XM_092489, 867
XM_092517, 676
XM_092545, 970
XM_092760, 5696
XM_092888, 5986
XM_092966, 6113
XM_093050, 6212
XM_093130, 6226
XM_093219, 6299
XM_093241, 6228
XM_093423, 1308
XM_093487, 1255
XM_093546, 1201
XM_093624, 1083
XM_094243, 1797
XM_094440, 1561
XM_094741, 1862
XM_094855, 2060
XM_095146, 2432
XM_095371, 2475
XM_095545, 2514
XM_095667, 2554
XM_096038, 3699
XM_096060, 4241
XM_096146, 3539
XM_096149, 661
XM_096155, 5967
XM_096156, 5968
XM_096169, 1022
XM_096172, 787
XM_096195, 1190
XM_096198, 1117
XM_096203, 1464
XM_096303, 6256
XM_096486, 3315
XM_096520, 3165
XM_096544, 3119
XM_096566, 3680
XM_096572, 3819
XM_096597, 3739
XM_096606, 3608
XM_096620, 3578
XM_096630, 3486
XM_096661, 3441
XM_096744, 4034
XM_096772, 3966
XM_096842, 4245
XM_096844, 4286
XM_097043, 4984
XM_097193, 5001
XM_097195, 5000
XM_097204, 4754
XM_097232, 5048
XM_097274, 5510
XM_097275, 5521
XM_097300, 5222
XM_097365, 5440
XM_097420, 5134
XM_097453, 2068
XM_097519, 561
XM_097565, 249
XM_097639, 352
XM_097649, 198
XM_097713, 5800
XM_097727, 5773
XM_097731, 5795
XM_097749, 5644
XM_097772, 5731
XM_097801, 5929
XM_097817, 5925
XM_097833, 5950
XM_097886, 5971
XM_097976, 715
XM_098004, 729
XM_098047, 962
XM_098048, 960
XM_098109, 1345
XM_098111, 1245
XM_098154, 1232
XM_098158, 1103
XM_098173, 1227
XM_098248, 1384
XM_098351, 1609
XM_098352, 1611
XM_098354, 1610
XM_098362, 1634
XM_098387, 1778
XM_098405, 1534
XM_098468, 2108
XM_098599, 619
XM_098654, 2447
XM_098669, 2466
XM_098747, 2582
XM_098761, 2564
XM_098913, 2843
XM_098943, 2725
XM_098995, 6302
XM_099467, 363
XM_102377, 4432
XM_103946, 665
XM_104983, 6263
XM_105236, 1289
XM_105658, 1325
XM_106246, 1520
XM_106739, 1562
XM_107825, 2225
XM_109162, 3075
XM_113223, 3268
XM_113224, 3275
XM_113226, 3400
XM_113229, 3366
XM_113230, 3363
XM_113238, 3152
XM_113266, 4202
XM_113268, 4207
XM_113291, 4429
XM_113293, 4467
XM_113299, 4504
XM_113303, 5013
XM_113310, 4723
XM_113315, 4944
XM_113324, 4674
XM_113325, 4703
XM_113328, 4695
XM_113330, 5011
XM_113334, 4819
XM_113343, 5028
XM_113348, 5316
XM_113352, 5294
XM_113360, 386
XM_113361, 598
XM_113369, 361
XM_113374, 140
XM_113379, 473
XM_113380, 5749
XM_113390, 929
XM_113395, 1193
XM_113397, 1244
XM_113405, 1140
XM_113408, 1296
XM_113409, 1202
XM_113410, 1088
XM_113417, 1254
XM_113422, 1329
XM_113425, 1452
XM_113452, 1556
XM_113454, 1841
XM_113463, 1654
XM_113467, 1720
XM_113468, 1845
XM_113476, 1860
XM_113531, 2526
XM_113532, 2627
XM_113540, 2548
XM_113557, 2493
XM_113564, 2846
XM_113585, 6122
XM_113615, 2927
XM_113702, 3862
XM_113712, 3635
XM_113719, 3560
XM_113726, 3584
XM_113730, 3519
XM_113737, 3855
XM_113739, 3437
XM_113752, 3946
XM_113759, 4105
XM_113823, 4163
XM_113836, 4326
XM_113840, 4608
XM_113843, 4420
XM_113845, 4418
XM_113853, 4570
XM_113855, 4560
XM_113874, 4431
XM_113876, 4426
XM_113882, 4640
XM_113892, 4978
XM_113901, 4653
XM_113919, 4905
XM_113929, 4696
XM_113931, 4706
XM_113938, 4824
XM_113943, 5010
XM_113945, 4998
XM_113951, 4962
XM_113988, 5229
XM_114004, 5349
XM_114018, 5097
XM_114024, 5560
XM_114025, 5530
XM_114027, 5366
XM_114030, 560
XM_114044, 129
XM_114055, 384
XM_114062, 3
XM_114097, 376
XM_114098, 360
XM_114109, 525
XM_114125, 259
XM_114137, 634
XM_114153, 484
XM_114154, 5875
XM_114163, 5794
XM_114165, 5813
XM_114174, 5673
XM_114178, 5706
XM_114185, 5889
XM_114209, 6024
XM_114215, 816
XM_114229, 838
XM_114247, 824
XM_114266, 851
XM_114267, 856
XM_114298, 957
XM_114301, 1225
XM_114309, 1242
XM_114323, 1141
XM_114328, 1344
XM_114356, 1288
XM_114364, 1122
XM_114368, 1510
XM_114401, 1496
XM_114424, 1473
XM_114426, 1470
XM_114434, 1555
XM_114435, 1552
XM_114437, 1567
XM_114439, 1586
XM_114440, 1587
XM_114442, 1584
XM_114453, 1819
XM_114457, 1817
XM_114469, 1623
XM_114482, 1683
XM_114492, 2106
XM_114497, 2058
XM_114555, 2429
XM_114578, 2444
XM_114602, 2404
XM_114613, 2625
XM_114617, 2517
XM_114618, 2523
XM_114640, 2556
XM_114646, 2756
XM_114649, 2873
XM_114655, 2854
XM_114661, 2677
XM_114662, 2688
XM_114669, 2845
XM_114677, 2802
XM_114678, 2801
XM_114679, 2799
XM_114686, 2699
XM_114692, 6354
XM_114708, 6291
XM_114720, 6130
XM_114724, 6119
XM_114798, 233
XM_114862, 3104
XM_114894, 2977
XM_114981, 3139
XM_115031, 3286
XM_115062, 3364
XM_115063, 3365
XM_115081, 3177
XM_115117, 3570
XM_115140, 3634
XM_115197, 3809
XM_115215, 3948
XM_115352, 4333
XM_115480, 4910
XM_115603, 5466
XM_115615, 5395
XM_115672, 869
XM_115706, 1039
XM_115722, 1040
XM_115825, 1002
XM_115846, 5691
XM_115874, 6281
XM_115886, 6131
XM_115890, 6136
XM_115923, 6259
XM_115924, 6121
XM_116034, 1338
XM_116058, 1295
XM_116071, 1204
XM_116072, 1205
XM_116204, 1532
XM_116205, 1533
XM_116247, 1484
XM_116285, 1408
XM_116307, 1691
XM_116340, 1807
XM_116365, 1856
XM_116427, 1648
XM_116439, 1593
XM_116447, 1606
XM_116465, 1716
XM_116511, 1857
XM_116514, 1861
XM_116524, 2140
XM_116806, 2789
XM_116818, 2738
XM_116853, 1139
XM_116856, 1810
XM_116863, 2975
XM_116913, 3845
XM_116926, 3451
XM_117061, 4913
XM_117066, 4768
XM_117096, 5084
XM_117118, 5379
XM_117122, 5183
XM_117128, 5605
XM_117159, 2
XM_117181, 534
XM_117184, 163
XM_117185, 582
XM_117196, 641
XM_117209, 5688
XM_117264, 736
XM_117311, 1337
XM_117351, 1412
XM_117387, 1622
XM_117398, 1641
XM_117444, 2471
XM_117449, 2160
XM_117452, 2472
XM_117481, 2406
XM_117487, 2622
XM_117519, 2874
XM_117539, 6352
XM_117555, 6349
XM_117692, 28
XM_118637, 4251
XM_165390, 3427
XM_165410, 4583
XM_165411, 4413
XM_165418, 4713
XM_165421, 4701
XM_165422, 4704
XM_165432, 5541
XM_165438, 144
XM_165439, 620
XM_165442, 59
XM_165443, 477
XM_165448, 723
XM_165451, 1268
XM_165465, 1531
XM_165470, 1528
XM_165473, 1482
XM_165483, 1818
XM_165484, 1820
XM_165488, 1615
XM_165499, 2057
XM_165514, 2579
XM_165530, 6355
XM_165533, 6235
XM_165551, 2913
XM_165555, 2889
XM_165557, 2897
XM_165560, 2925
XM_165563, 2926
XM_165567, 2921
XM_165571, 3407
XM_165584, 3414
XM_165586, 3413
XM_165592, 3401
XM_165598, 3303
XM_165600, 3310
XM_165610, 3222
XM_165611, 3217
XM_165612, 3223
XM_165616, 3325
XM_165627, 3335
XM_165628, 3341
XM_165631, 3328
XM_165636, 3903
XM_165639, 3917
XM_165645, 4534
XM_165647, 4528
XM_165648, 4537
XM_165649, 4527
XM_165656, 4484
XM_165657, 4493
XM_165658, 4489
XM_165669, 2091
XM_165692, 2159
XM_165698, 1949
XM_165717, 1954
XM_165728, 2036
XM_165738, 1999
XM_165740, 1865
XM_165743, 1937
XM_165747, 1948
XM_165749, 2037
XM_165758, 2013
XM_165764, 2011
XM_165765, 1988
XM_165770, 1951
XM_165771, 1983
XM_165772, 1876
XM_165777, 2044
XM_165794, 1921
XM_165799, 2006
XM_165801, 1956
XM_165809, 2016
XM_165836, 2350
XM_165839, 2346
XM_165841, 2197
XM_165860, 2167
XM_165867, 2249
XM_165870, 2245
XM_165872, 2253
XM_165876, 2258
XM_165877, 2240
XM_165882, 2248
XM_165888, 2934
XM_165890, 2929
XM_165891, 2941
XM_165903, 3633
XM_165905, 3579
XM_165906, 3532
XM_165910, 3465
XM_165921, 4127
XM_165923, 4325
XM_165954, 5026
XM_165960, 5347
XM_165963, 5367
XM_165975, 327
XM_165976, 373
XM_165977, 264
XM_165978, 532
XM_165981, 290
XM_165983, 275
XM_165984, 175
XM_165994, 927
XM_165998, 893
XM_166007, 910
XM_166008, 900
XM_166011, 1121
XM_166014, 1275
XM_166015, 1192
XM_166017, 1350
XM_166026, 1669
XM_166027, 1663
XM_166028, 1842
XM_166029, 1802
XM_166037, 1612
XM_166042, 2054
XM_166049, 2147
XM_166063, 2540
XM_166064, 2558
XM_166078, 6142
XM_166081, 6255
XM_166093, 2984
XM_166125, 2966
XM_166157, 2922
XM_166174, 3409
XM_166177, 3406
XM_166181, 3403
XM_166196, 3308
XM_166232, 3227
XM_166234, 3224
XM_166235, 3293
XM_166236, 3294
XM_166239, 3349
XM_166253, 3336
XM_166266, 3904
XM_166273, 3886
XM_166277, 4532
XM_166282, 4491
XM_166285, 4490
XM_166288, 5071
XM_166303, 2092
XM_166310, 2101
XM_166327, 2157
XM_166333, 1932
XM_166336, 2021
XM_166340, 1882
XM_166349, 1872
XM_166353, 2002
XM_166357, 2049
XM_166360, 1938
XM_166361, 2009
XM_166362, 1884
XM_166363, 1940
XM_166376, 2004
XM_166381, 1992
XM_166392, 2019
XM_166401, 1995
XM_166402, 1896
XM_166406, 2015
XM_166412, 1910
XM_166417, 1914
XM_166419, 1920
XM_166425, 1888
XM_166446, 2042
XM_166457, 1878
XM_166459, 1931
XM_166469, 1879
XM_166480, 1955
XM_166482, 2351
XM_166485, 2353
XM_166494, 2224
XM_166504, 2222
XM_166505, 2202
XM_166506, 2200
XM_166509, 2219
XM_166512, 2205
XM_166513, 2220
XM_166514, 2203
XM_166515, 2204
XM_166521, 2198
XM_166523, 2170
XM_166531, 2190
XM_166540, 2191
XM_166541, 2168
XM_166594, 2230
XM_166599, 20
XM_166605, 3506
XM_166629, 2988
XM_166665, 2918
XM_166717, 2906
XM_166743, 3418
XM_167008, 5080
XM_167016, 2087
XM_167027, 2094
XM_167037, 2096
XM_167046, 2150
XM_167128, 2023
XM_167161, 2025
XM_167169, 1868
XM_167179, 2031
XM_167196, 2041
XM_167225, 2047
XM_167339, 2264
XM_167363, 5065
XM_167366, 1209
XM_167374, 2898
XM_167395, 2963
XM_167411, 2901
XM_167414, 2904
XM_167433, 3324
XM_167437, 3192
XM_167439, 3876
XM_167453, 4538
XM_167456, 4541
XM_167476, 2321
XM_167477, 2325
XM_167483, 2328
XM_167484, 2329
XM_167494, 2273
XM_167498, 2301
XM_167500, 2299
XM_167502, 2312
XM_167504, 2300
XM_167518, 3754
XM_167530, 5529
XM_167538, 5945
XM_167558, 2645
XM_167626, 2887
XM_167716, 3244
XM_167726, 3248
XM_167747, 3234
XM_167748, 3228
XM_167780, 3417
XM_167804, 3291
XM_167853, 3318
XM_167892, 3883
XM_167906, 3877
XM_167911, 3868
XM_167918, 3869
XM_168054, 2103
XM_168070, 1928
XM_168104, 1994
XM_168123, 1877
XM_168181, 2322
XM_168251, 2323
XM_168354, 2271
XM_168378, 2269
XM_168435, 2316
XM_168450, 2315
XM_168454, 2302
XM_168461, 2311
XM_168464, 2317
XM_168470, 2310
XM_168548, 2375
XM_168572, 2380
XM_168586, 2360
XM_169414, 3880
XM_169540, 5078
XM_170195, 2267
XM_170427, 2318

Source Index (to Figure number)

gen.NM_000018,4669
gen.NM_000026,6068
gen.NM_000029,624
gen.NM_000033,6342
gen.NM_000034,4520
gen.NM_000039,3376
gen.NM_000041,5511
gen.NM_000070,4161
gen.NM_000075,3683
gen.NM_000077,2655
gen.NM_000079,898
gen.NM_000090,921
gen.NM_000107,3208
gen.NM_000114,5836
gen.NM_000121,5258
gen.NM_000126,4267
gen.NM_000137,4300
gen.NM_000143,636
gen.NM_000146,5562
gen.NM_000154,4967
gen.NM_000156,5122
gen.NM_000165,2099
gen.NM_000177,2796
gen.NM_000178,5738
gen.NM_000179,744
gen.NM_000182,713
gen.NM_000183,711
gen.NM_000184,3144
gen.NM_000196,4547
gen.NM_000213,4963
gen.NM_000221,701
gen.NM_000224,3593
gen.NM_000227,5040
gen.NM_000228,553
gen.NM_000239,3729
gen.NM_000250,4903
gen.NM_000251,741
gen.NM_000268,5994
gen.NM_000269,4889
gen.NM_000274,3076
gen.NM_000284,6138
gen.NM_000291,6230
gen.NM_000358,1671
gen.NM_000365,3460
gen.NM_000368,2806
gen.NM_000385,2262
gen.NM_000386,4843
gen.NM_000396,356
gen.NM_000404,1089
gen.NM_000407,5947
gen.NM_000422,4807
gen.NM_000425,6334
gen.NM_000447,594
gen.NM_000484,5882
gen.NM_000505,1828
gen.NM_000508,1511
gen.NM_000509,1515
gen.NM_000516,5830
gen.NM_000517,4354
gen.NM_000521,1627
gen.NM_000526,4816
gen.NM_000532,1260
gen.NM_000554,5480
gen.NM_000558,4356
gen.NM_000559,3142
gen.NM_000569,505
gen.NM_000574,558
gen.NM_000576,847
gen.NM_000582,1459
gen.NM_000592,1957
gen.NM_000598,2228
gen.NM_000602,2361
gen.NM_000612,3120
gen.NM_000638,4763
gen.NM_000661,1425
gen.NM_000666,1172
gen.NM_000687,5736
gen.NM_000688,1167
gen.NM_000700,2695
gen.NM_000701,312
gen.NM_000743,4259
gen.NM_000754,5956
gen.NM_000760,173
gen.NM_000785,3687
gen.NM_000787,2830
gen.NM_000795,3384
gen.NM_000801,5648
gen.NM_000852,3297
gen.NM_000858,612
gen.NM_000893,1327
gen.NM_000895,3763
gen.NM_000930,2534
gen.NM_000931,2536
gen.NM_000942,4218
gen.NM_000954,2868
gen.NM_000964,4820
gen.NM_000967,6061
gen.NM_000969,284
gen.NM_000970,3781
gen.NM_000971,2569
gen.NM_000972,2826
gen.NM_000973,2633
gen.NM_000975,87
gen.NM_000976,2780
gen.NM_000977,4633
gen.NM_000978,4801
gen.NM_000979,5571
gen.NM_000980,5334
gen.NM_000981,4798
gen.NM_000982,3091
gen.NM_000983,34
gen.NM_000985,5067
gen.NM_000986,1206
gen.NM_000987,4714
gen.NM_000989,2588
gen.NM_000990,3155
gen.NM_000991,5613
gen.NM_000992,1170
gen.NM_000993,832
gen.NM_000994,1064
gen.NM_000997,1570
gen.NM_000998,966
gen.NM_001000,6278
gen.NM_001002,3827
gen.NM_001003,4228
gen.NM_001005,3331
gen.NM_001006,1506
gen.NM_001007,6224
gen.NM_001009,5633
gen.NM_001010,2651
gen.NM_001011,643
gen.NM_001012,210
gen.NM_001016,2111
gen.NM_001017,3171
gen.NM_001018,5126
gen.NM_001020,5426
gen.NM_001021,4283
gen.NM_001022,5468
gen.NM_001023,2552
gen.NM_001024,5847
gen.NM_001025,1632
gen.NM_001026,2980
gen.NM_001028,3361
gen.NM_001029,3656
gen.NM_001030,440
gen.NM_001034,651
gen.NM_001038,3478
gen.NM_001043,4487
gen.NM_001050,4841
gen.NM_001064,1159
gen.NM_001065,3480
gen.NM_001068,1079
gen.NM_001069,2050
gen.NM_001084,2369
gen.NM_001087,994
gen.NM_001098,6079
gen.NM_001101,2174
gen.NM_001102,4040
gen.NM_001122,2649
gen.NM_001134,1446
gen.NM_001154,1489
gen.NM_001157,2990
gen.NM_001168,4985
gen.NM_001190,5568
gen.NM_001199,2495
gen.NM_001207,1624
gen.NM_001211,4139
gen.NM_001218,4203
gen.NM_001235,3333
gen.NM_001238,5374
gen.NM_001247,5703
gen.NM_001255,194
gen.NM_001262,229
gen.NM_001273,3468
gen.NM_001274,3411
gen.NM_001275,4065
gen.NM_001283,2365
gen.NM_001287,4372
gen.NM_001288,1969
gen.NM_001293,3337
gen.NM_001294,5508
gen.NM_001313,1396
gen.NM_001319,5141
gen.NM_001320,1971
gen.NM_001324,5814
gen.NM_001325,6239
gen.NM_001333,2736
gen.NM_001344,3984
gen.NM_001350,1942
gen.NM_001363,6318
gen.NM_001407,1132
gen.NM_001415,6143
gen.NM_001416,4687
gen.NM_001418,3163
gen.NM_001428,31
gen.NM_001436,5436
gen.NM_001444,2575
gen.NM_001450,836
gen.NM_001463,916
gen.NM_001465,1573
gen.NM_001467,3359
gen.NM_001469,6081
gen.NM_001494,2891
gen.NM_001500,2052
gen.NM_001517,1997
gen.NM_001521,689
gen.NM_001530,4016
gen.NM_001536,5539
gen.NM_001539,2660
gen.NM_001540,2308
gen.NM_001553,1435
gen.NM_001554,269
gen.NM_001560,6270
gen.NM_001567,3322
gen.NM_001568,2596
gen.NM_001569,6332
gen.NM_001571,5542
gen.NM_001605,4564
gen.NM_001607,1097
gen.NM_001610,3206
gen.NM_001613,3008
gen.NM_001622,1330
gen.NM_001628,2423
gen.NM_001641,3997
gen.NM_001644,3511
gen.NM_001647,1352
gen.NM_001648,5590
gen.NM_001659,3550
gen.NM_001662,2398
gen.NM_001667,3284
gen.NM_001673,2355
gen.NM_001687,5115
gen.NM_001688,308
gen.NM_001696,5941
gen.NM_001697,5892
gen.NM_001710,1959
gen.NM_001734,3452
gen.NM_001743,5494
gen.NM_001747,806
gen.NM_001751,3137
gen.NM_001753,2391
gen.NM_001757,5894
gen.NM_001760,1898
gen.NM_001762,2274
gen.NM_001780,3663
gen.NM_001791,81
gen.NM_001816,5478
gen.NM_001819,5679
gen.NM_001827,2714
gen.NM_001831,2506
gen.NM_001833,2689
gen.NM_001842,2668
gen.NM_001853,5853
gen.NM_001861,4614
gen.NM_001862,827
gen.NM_001878,392
gen.NM_001907,4579
gen.NM_001909,3133
gen.NM_001920,3740
gen.NM_001930,5267
gen.NM_001935,894
gen.NM_001944,5050
gen.NM_001959,950
gen.NM_001961,5178
gen.NM_001964,1689
gen.NM_001969,4098
gen.NM_001970,4697
gen.NM_001975,3458
gen.NM_001983,5502
gen.NM_001985,5593
gen.NM_002003,2834
gen.NM_002004,422
gen.NM_002011,1836
gen.NM_002014,3439
gen.NM_002015,3896
gen.NM_002018,4719
gen.NM_002028,4010
gen.NM_002046,3473
gen.NM_002047,2265
gen.NM_002075,3463
gen.NM_002079,3066
gen.NM_002083,4012
gen.NM_002084,1704
gen.NM_002085,5112
gen.NM_002086,4953
gen.NM_002087,4845
gen.NM_002106,1478
gen.NM_002109,1779
gen.NM_002128,3887
gen.NM_002129,1522
gen.NM_002130,1582
gen.NM_002133,6020
gen.NM_002137,2210
gen.NM_002157,930
gen.NM_002161,2716
gen.NM_002168,4293
gen.NM_002178,3600
gen.NM_002211,2919
gen.NM_002212,5742
gen.NM_002229,5272
gen.NM_002265,4834
gen.NM_002273,3591
gen.NM_002274,4814
gen.NM_002275,4812
gen.NM_002276,4810
gen.NM_002295,1108
gen.NM_002305,6038
gen.NM_002306,4022
gen.NM_002339,3115
gen.NM_002340,5931
gen.NM_002342,3476
gen.NM_002345,3752
gen.NM_002355,3489
gen.NM_002358,1485
gen.NM_002364,6147
gen.NM_002385,5086
gen.NM_002386,4626
gen.NM_002388,1866
gen.NM_002396,5069
gen.NM_002397,1646
gen.NM_002401,4933
gen.NM_002411,3245
gen.NM_002413,1494
gen.NM_002414,6124
gen.NM_002415,5979
gen.NM_002453,751
gen.NM_002466,5774
gen.NM_002468,1095
gen.NM_002473,6025
gen.NM_002477,1368
gen.NM_002484,4416
gen.NM_002486,2734
gen.NM_002489,2193
gen.NM_002492,1297
gen.NM_002512,4887
gen.NM_002520,1803
gen.NM_002537,4210
gen.NM_002539,659
gen.NM_002567,3816
gen.NM_002568,2593
gen.NM_002574,220
gen.NM_002588,1728
gen.NM_002606,5900
gen.NM_002615,4647
gen.NM_002617,12
gen.NM_002632,4052
gen.NM_002634,4939
gen.NM_002638,5779
gen.NM_002654,4242
gen.NM_002660,5771
gen.NM_002668,6185
gen.NM_002689,3289
gen.NM_002691,5580
gen.NM_002707,681
gen.NM_002712,1030
gen.NM_002720,4518
gen.NM_002727,2961
gen.NM_002730,5298
gen.NM_002733,3555
gen.NM_002766,4975
gen.NM_002787,2254
gen.NM_002789,4261
gen.NM_002792,5838
gen.NM_002793,2137
gen.NM_002796,346
gen.NM_002802,4059
gen.NM_002803,2378
gen.NM_002809,4805
gen.NM_002810,348
gen.NM_002812,5401
gen.NM_002813,3837
gen.NM_002815,4778
gen.NM_002819,5102
gen.NM_002827,5809
gen.NM_002846,980
gen.NM_002854,1188
gen.NM_002856,5515
gen.NM_002857,481
gen.NM_002863,4029
gen.NM_002870,438
gen.NM_002878,4784
gen.NM_002883,6075
gen.NM_002887,1800
gen.NM_002913,1427
gen.NM_002915,3891
gen.NM_002921,3002
gen.NM_002923,540
gen.NM_002934,3992
gen.NM_002938,1386
gen.NM_002946,127
gen.NM_002947,2188
gen.NM_002948,1076
gen.NM_002952,4382
gen.NM_002954,749
gen.NM_002961,369
gen.NM_002965,364
gen.NM_002979,235
gen.NM_003002,3390
gen.NM_003021,5161
gen.NM_003025,5188
gen.NM_003055,2947
gen.NM_003064,5781
gen.NM_003072,5254
gen.NM_003076,3568
gen.NM_003088,2176
gen.NM_003090,4320
gen.NM_003091,5654
gen.NM_003092,5683
gen.NM_003104,4187
gen.NM_003107,2032
gen.NM_003123,4511
gen.NM_003124,789
gen.NM_003128,746
gen.NM_003132,50
gen.NM_003137,1916
gen.NM_003143,2435
gen.NM_003145,409
gen.NM_003146,3215
gen.NM_003149,1099
gen.NM_003169,5428
gen.NM_003181,2135
gen.NM_003216,6077
gen.NM_003283,5608
gen.NM_003287,2104
gen.NM_003289,2680
gen.NM_003290,5312
gen.NM_003295,3900
gen.NM_003310,649
gen.NM_003316,5896
gen.NM_003334,6167
gen.NM_003349,5804
gen.NM_003350,2546
gen.NM_003365,1134
gen.NM_003366,4421
gen.NM_003370,5499
gen.NM_003374,1677
gen.NM_003375,2982
gen.NM_003378,2367
gen.NM_003389,2728
gen.NM_003400,761
gen.NM_003401,1636
gen.NM_003406,2590
gen.NM_003418,1250
gen.NM_003453,3864
gen.NM_003461,2440
gen.NM_003472,2034
gen.NM_003516,459
gen.NM_003564,474
gen.NM_003598,5556
gen.NM_003617,497
gen.NM_003624,5214
gen.NM_003626,3316
gen.NM_003646,3197
gen.NM_003662,6149
gen.NM_003680,157
gen.NM_003681,5905
gen.NM_003685,5203
gen.NM_003687,1673
gen.NM_003689,71
gen.NM_003712,5093
gen.NM_003714,1812
gen.NM_003720,5898
gen.NM_003721,5360
gen.NM_003722,1335
gen.NM_003729,288
gen.NM_003735,1730
gen.NM_003736,1732
gen.NM_003739,2883
gen.NM_003752,4449
gen.NM_003753,6027
gen.NM_003755,5234
gen.NM_003756,2598
gen.NM_003757,148
gen.NM_003765,5288
gen.NM_003766,4865
gen.NM_003779,468
gen.NM_003780,199
gen.NM_003787,5052
gen.NM_003815,457
gen.NM_003824,3313
gen.NM_003836,4088
gen.NM_003837,2723
gen.NM_003859,5811
gen.NM_003876,4708
gen.NM_003877,3757
gen.NM_003906,5933
gen.NM_003908,5734
gen.NM_003915,5747
gen.NM_003932,6070
gen.NM_003937,881
gen.NM_003938,5148
gen.NM_003971,4891
gen.NM_003973,1110
gen.NM_003979,3498
gen.NM_004000,306
gen.NM_004004,3866
gen.NM_004044,955
gen.NM_004048,4178
gen.NM_004053,1900
gen.NM_004060,1791
gen.NM_004074,3264
gen.NM_004084,2476
gen.NM_004085,6242
gen.NM_004092,3099
gen.NM_004111,3253
gen.NM_004117,1918
gen.NM_004127,5008
gen.NM_004134,1693
gen.NM_004135,6340
gen.NM_004147,6011
gen.NM_004152,5154
gen.NM_004159,1952
gen.NM_004175,5983
gen.NM_004176,4742
gen.NM_004178,3614
gen.NM_004181,1430
gen.NM_004182,6174
gen.NM_004193,3045
gen.NM_004203,4402
gen.NM_004208,6285
gen.NM_004217,4699
gen.NM_004219,1795
gen.NM_004240,5206
gen.NM_004247,4879
gen.NM_004261,273
gen.NM_004265,3249
gen.NM_004309,5002
gen.NM_004322,3256
gen.NM_004323,2662
gen.NM_004324,5564
gen.NM_004335,5328
gen.NM_004339,5921
gen.NM_004341,692
gen.NM_004345,1128
gen.NM_004360,4549
gen.NM_004398,3392
gen.NM_004401,48
gen.NM_004404,1034
gen.NM_004435,2761
gen.NM_004448,4796
gen.NM_004461,5279
gen.NM_004483,4602
gen.NM_004493,6190
gen.NM_004509,1012
gen.NM_004510,1014
gen.NM_004524,4960
gen.NM_004539,5072
gen.NM_004547,1218
gen.NM_004550,470
gen.NM_004551,3199
gen.NM_004555,4586
gen.NM_004573,4141
gen.NM_004595,6140
gen.NM_004596,5448
gen.NM_004599,6085
gen.NM_004618,4716
gen.NM_004632,414
gen.NM_004635,1155
gen.NM_004636,1149
gen.NM_004637,1246
gen.NM_004638,1979
gen.NM_004639,1973
gen.NM_004640,1986
gen.NM_004673,529
gen.NM_004691,4545
gen.NM_004697,2751
gen.NM_004699,6323
gen.NM_004701,4197
gen.NM_004704,1182
gen.NM_004706,5470
gen.NM_004714,5434
gen.NM_004725,3093
gen.NM_004728,2959
gen.NM_004735,1026
gen.NM_004738,5824
gen.NM_004739,3230
gen.NM_004766,1270
gen.NM_004767,576
gen.NM_004772,1650
gen.NM_004781,44
gen.NM_004794,6287
gen.NM_004813,3190
gen.NM_004821,1787
gen.NM_004844,1066
gen.NM_004846,998
gen.NM_004859,4921
gen.NM_004870,4689
gen.NM_004889,2342
gen.NM_004893,1685
gen.NM_004905,511
gen.NM_004911,2442
gen.NM_004928,5915
gen.NM_004930,69
gen.NM_004933,4638
gen.NM_004939,662
gen.NM_004957,2775
gen.NM_004960,4465
gen.NM_004964,150
gen.NM_004973,2039
gen.NM_004982,3526
gen.NM_004990,3669
gen.NM_004992,6330
gen.NM_004994,5791
gen.NM_004995,3976
gen.NM_005000,2396
gen.NM_005002,3448
gen.NM_005003,4446
gen.NM_005004,3063
gen.NM_005005,2606
gen.NM_005008,6083
gen.NM_005015,3981
gen.NM_005016,3620
gen.NM_005022,4665
gen.NM_005030,4442
gen.NM_005036,6104
gen.NM_005042,3524
gen.NM_005053,5283
gen.NM_005072,4581
gen.NM_005080,5987
gen.NM_005109,1093
gen.NM_005110,1854
gen.NM_005112,1421
gen.NM_005115,4500
gen.NM_005132,3962
gen.NM_005141,1508
gen.NM_005163,4110
gen.NM_005171,3574
gen.NM_005174,2895
gen.NM_005194,5808
gen.NM_005217,2478
gen.NM_005220,4946
gen.NM_005224,5104
gen.NM_005243,5989
gen.NM_005269,3667
gen.NM_005271,3004
gen.NM_005291,854
gen.NM_005300,6159
gen.NM_005313,4174
gen.NM_005324,4969
gen.NM_005330,3146
gen.NM_005333,6126
gen.NM_005345,1963
gen.NM_005346,1961
gen.NM_005347,2790
gen.NM_005348,4092
gen.NM_005362,6316
gen.NM_005364,6308
gen.NM_005370,5314
gen.NM_005371,3689
gen.NM_005378,657
gen.NM_005389,2126
gen.NM_005432,4101
gen.NM_005439,3466
gen.NM_005440,4877
gen.NM_005452,1944
gen.NM_005474,4850
gen.NM_005490,5208
gen.NM_005498,5241
gen.NM_005514,2155
gen.NM_005517,110
gen.NM_005520,1850
gen.NM_005548,4568
gen.NM_005563,105
gen.NM_005566,3175
gen.NM_005572,404
gen.NM_005573,1718
gen.NM_005581,5517
gen.NM_005594,3628
gen.NM_005614,2460
gen.NM_005617,1708
gen.NM_005620,340
gen.NM_005623,4782
gen.NM_005632,4362
gen.NM_005657,4170
gen.NM_005663,1382
gen.NM_005676,6165
gen.NM_005686,550
gen.NM_005692,2458
gen.NM_005693,3204
gen.NM_005698,424
gen.NM_005710,6181
gen.NM_005713,1602
gen.NM_005717,517
gen.NM_005718,1055
gen.NM_005720,2348
gen.NM_005724,4273
gen.NM_005726,3695
gen.NM_005729,2986
gen.NM_005731,996
gen.NM_005745,6344
gen.NM_005754,1697
gen.NM_005762,5627
gen.NM_005770,4176
gen.NM_005775,2491
gen.NM_005783,829
gen.NM_005787,1316
gen.NM_005796,4575
gen.NM_005806,5887
gen.NM_005826,83
gen.NM_005830,3898
gen.NM_005831,4911
gen.NM_005833,2792
gen.NM_005837,2326
gen.NM_005850,461
gen.NM_005851,3301
gen.NM_005855,1024
gen.NM_005866,2670
gen.NM_005877,5999
gen.NM_005884,5421
gen.NM_005889,3509
gen.NM_005911,808
gen.NM_005915,864
gen.NM_005917,764
gen.NM_005918,2306
gen.NM_005973,389
gen.NM_005981,3681
gen.NM_005983,1579
gen.NM_005985,5802
gen.NM_005997,350
gen.NM_006000,982
gen.NM_006012,5201
gen.NM_006013,6326
gen.NM_006019,3304
gen.NM_006023,2899
gen.NM_006039,4936
gen.NM_006053,3306
gen.NM_006058,1702
gen.NM_006066,218
gen.NM_006067,4612
gen.NM_006098,1852
gen.NM_006101,5023
gen.NM_006109,3973
gen.NM_006110,4423
gen.NM_006112,159
gen.NM_006114,5513
gen.NM_006115,5975
gen.NM_006128,2497
gen.NM_006131,2499
gen.NM_006132,2501
gen.NM_006136,2393
gen.NM_006169,3380
gen.NM_006184,5566
gen.NM_006227,5789
gen.NM_006230,2246
gen.NM_006245,1892
gen.NM_006247,5497
gen.NM_006250,3522
gen.NM_006253,3831
gen.NM_006262,3546
gen.NM_006265,2600
gen.NM_006271,374
gen.NM_006272,5935
gen.NM_006280,6338
gen.NM_006289,2682
gen.NM_006295,1967
gen.NM_006303,2178
gen.NM_006330,2550
gen.NM_006335,571
gen.NM_006339,5171
gen.NM_006342,1374
gen.NM_006349,2371
gen.NM_006354,1049
gen.NM_006362,3242
gen.NM_006365,396
gen.NM_006373,4875
gen.NM_006384,4305
gen.NM_006387,5319
gen.NM_006395,1062
gen.NM_006397,5277
gen.NM_006401,2732
gen.NM_006427,4106
gen.NM_006428,4360
gen.NM_006429,792
gen.NM_006430,759
gen.NM_006432,4048
gen.NM_006435,3113
gen.NM_006439,1504
gen.NM_006440,5954
gen.NM_006453,4384
gen.NM_006455,4822
gen.NM_006470,4725
gen.NM_006478,5991
gen.NM_006488,703
gen.NM_006494,5476
gen.NM_006503,5441
gen.NM_006513,298
gen.NM_006516,188
gen.NM_006523,3055
gen.NM_006530,3727
gen.NM_006556,452
gen.NM_006559,146
gen.NM_006576,3697
gen.NM_006585,5885
gen.NM_006586,1894
gen.NM_006589,428
gen.NM_006600,118
gen.NM_006601,3636
gen.NM_006621,300
gen.NM_006625,93
gen.NM_006636,794
gen.NM_006646,3881
gen.NM_006659,3101
gen.NM_006666,5558
gen.NM_006667,6272
gen.NM_006670,2070
gen.NM_006693,2344
gen.NM_006694,436
gen.NM_006698,5760
gen.NM_006708,1904
gen.NM_006711,4392
gen.NM_006746,6134
gen.NM_006761,4642
gen.NM_006763,548
gen.NM_006764,1151
gen.NM_006769,271
gen.NM_006787,6197
gen.NM_006791,4279
gen.NM_006799,4408
gen.NM_006801,5576
gen.NM_006805,1687
gen.NM_006808,2740
gen.NM_006810,1223
gen.NM_006812,3678
gen.NM_006815,3847
gen.NM_006816,1830
gen.NM_006817,3785
gen.NM_006821,4046
gen.NM_006824,192
gen.NM_006825,3807
gen.NM_006826,655
gen.NM_006833,2338
gen.NM_006835,1449
gen.NM_006837,2565
gen.NM_006839,814
gen.NM_006842,3295
gen.NM_006844,5308
gen.NM_006854,2184
gen.NM_006862,344
gen.NM_006888,4063
gen.NM_006899,5661
gen.NM_006908,2182
gen.NM_006924,4908
gen.NM_006928,3660
gen.NM_006932,6007
gen.NM_006938,5039
gen.NM_006941,6049
gen.NM_006942,4691
gen.NM_006990,124
gen.NM_007002,5844
gen.NM_007019,5785
gen.NM_007032,6040
gen.NM_007034,267
gen.NM_007046,705
gen.NM_007047,2029
gen.NM_007062,3805
gen.NM_007065,5237
gen.NM_007074,4516
gen.NM_007085,1216
gen.NM_007096,2691
gen.NM_007100,1366
gen.NM_007103,3299
gen.NM_007104,1922
gen.NM_007158,302
gen.NM_007165,5152
gen.NM_007173,3348
gen.NM_007178,3501
gen.NM_007184,1165
gen.NM_007186,5744
gen.NM_007190,3089
gen.NM_007209,2794
gen.NM_007242,4566
gen.NM_007244,3520
gen.NM_007260,89
gen.NM_007262,42
gen.NM_007263,5352
gen.NM_007268,6204
gen.NM_007273,3455
gen.NM_007275,1153
gen.NM_007276,2214
gen.NM_007279,5619
gen.NM_007310,5958
gen.NM_007311,6095
gen.NM_007317,4507
gen.NM_007355,1874
gen.NM_007364,4277
gen.NM_007372,4931
gen.NM_012068,5525
gen.NM_012098,2782
gen.NM_012099,5504
gen.NM_012100,977
gen.NM_012101,3420
gen.NM_012111,4055
gen.NM_012112,5715
gen.NM_012116,5519
gen.NM_012138,4838
gen.NM_012170,4265
gen.NM_012179,6017
gen.NM_012181,5350
gen.NM_012203,2693
gen.NM_012207,2955
gen.NM_012237,5409
gen.NM_012248,4451
gen.NM_012255,5698
gen.NM_012264,6054
gen.NM_012286,6246
gen.NM_012296,3344
gen.NM_012323,6052
gen.NM_012391,1929
gen.NM_012412,2236
gen.NM_012423,5550
gen.NM_012437,381
gen.NM_012458,5155
gen.NM_012469,5873
gen.NM_012486,596
gen.NM_013237,1834
gen.NM_013247,801
gen.NM_013265,3279
gen.NM_013274,3037
gen.NM_013277,3566
gen.NM_013296,292
gen.NM_013333,5617
gen.NM_013336,1238
gen.NM_013341,903
gen.NM_013363,1276
gen.NM_013365,6032
gen.NM_013369,5911
gen.NM_013375,2027
gen.NM_013393,2165
gen.NM_013402,3251
gen.NM_013403,5492
gen.NM_013406,5269
gen.NM_013407,5270
gen.NM_013417,2718
gen.NM_013442,2675
gen.NM_013451,3013
gen.NM_014003,4592
gen.NM_014008,6187
gen.NM_014033,3576
gen.NM_014035,1664
gen.NM_014042,3320
gen.NM_014062,4556
gen.NM_014063,2251
gen.NM_014107,2077
gen.NM_014138,6163
gen.NM_014166,3906
gen.NM_014172,2862
gen.NM_014173,5326
gen.NM_014176,578
gen.NM_014184,585
gen.NM_014188,17
gen.NM_014189,1390
gen.NM_014190,1388
gen.NM_014203,5536
gen.NM_014214,5032
gen.NM_014226,4095
gen.NM_014236,626
gen.NM_014248,6072
gen.NM_014255,3631
gen.NM_014267,3173
gen.NM_014275,1846
gen.NM_014285,2820
gen.NM_014294,2567
gen.NM_014303,6003
gen.NM_014306,6015
gen.NM_014311,3606
gen.NM_014320,2116
gen.NM_014321,4476
gen.NM_014325,3777
gen.NM_014335,4182
gen.NM_014341,1906
gen.NM_014353,4386
gen.NM_014408,167
gen.NM_014413,2180
gen.NM_014426,5685
gen.NM_014444,4168
gen.NM_014445,1284
gen.NM_014452,1870
gen.NM_014453,5625
gen.NM_014481,6199
gen.NM_014501,5615
gen.NM_014502,3220
gen.NM_014515,3724
gen.NM_014556,1394
gen.NM_014571,142
gen.NM_014585,923
gen.NM_014587,4370
gen.NM_014610,3232
gen.NM_014624,367
gen.NM_014649,5199
gen.NM_014663,202
gen.NM_014670,934
gen.NM_014685,4530
gen.NM_014713,667
gen.NM_014736,4214
gen.NM_014737,5676
gen.NM_014742,5721
gen.NM_014747,180
gen.NM_014748,684
gen.NM_014752,3329
gen.NM_014773,1721
gen.NM_014776,3792
gen.NM_014778,3878
gen.NM_014800,2259
gen.NM_014814,1195
gen.NM_014829,1681
gen.NM_014837,519
gen.NM_014847,446
gen.NM_014849,463
gen.NM_014851,36
gen.NM_014868,3823
gen.NM_014887,3889
gen.NM_014919,1378
gen.NM_014931,5610
gen.NM_014933,1457
gen.NM_014941,6005
gen.NM_014972,4628
gen.NM_015043,1843
gen.NM_015062,3042
gen.NM_015064,3430
gen.NM_015068,2319
gen.NM_015129,6276
gen.NM_015140,6097
gen.NM_015179,3024
gen.NM_015322,4226
gen.NM_015324,3149
gen.NM_015373,6056
gen.NM_015388,1886
gen.NM_015438,3470
gen.NM_015449,444
gen.NM_015453,1043
gen.NM_015472,1282
gen.NM_015484,99
gen.NM_015511,5752
gen.NM_015533,3225
gen.NM_015544,4780
gen.NM_015584,4761
gen.NM_015629,5600
gen.NM_015636,686
gen.NM_015640,260
gen.NM_015644,1057
gen.NM_015646,3720
gen.NM_015665,3604
gen.NM_015702,885
gen.NM_015714,555
gen.NM_015853,3238
gen.NM_015920,4205
gen.NM_015932,3884
gen.NM_015934,941
gen.NM_015937,5783
gen.NM_015953,5546
gen.NM_015965,5362
gen.NM_015966,5745
gen.NM_016003,2172
gen.NM_016016,4847
gen.NM_016022,334
gen.NM_016026,4037
gen.NM_016030,647
gen.NM_016059,1908
gen.NM_016085,694
gen.NM_016091,6045
gen.NM_016095,4610
gen.NM_016111,4374
gen.NM_016119,3912
gen.NM_016143,5652
gen.NM_016169,3051
gen.NM_016174,2767
gen.NM_016176,26
gen.NM_016183,73
gen.NM_016202,5621
gen.NM_016223,3210
gen.NM_016249,6300
gen.NM_016263,5169
gen.NM_016267,6293
gen.NM_016286,5006
gen.NM_016292,4414
gen.NM_016304,4193
gen.NM_016328,2293
gen.NM_016357,3572
gen.NM_016359,4152
gen.NM_016361,328
gen.NM_016410,2664
gen.NM_016440,5523
gen.NM_016445,4035
gen.NM_016456,564
gen.NM_016498,6001
gen.NM_016526,3107
gen.NM_016539,5181
gen.NM_016558,5750
gen.NM_016567,3097
gen.NM_016579,5216
gen.NM_016587,2216
gen.NM_016592,5826
gen.NM_016638,3843
gen.NM_016639,4398
gen.NM_016641,4335
gen.NM_016645,4302
gen.NM_016647,2614
gen.NM_016732,5733
gen.NM_016838,887
gen.NM_016839,889
gen.NM_016930,1400
gen.NM_016940,5883
gen.NM_016941,5432
gen.NM_017443,2753
gen.NM_017458,4498
gen.NM_017491,1419
gen.NM_017546,834
gen.NM_017566,4617
gen.NM_017572,5146
gen.NM_017595,4871
gen.NM_017601,1902
gen.NM_017610,4195
gen.NM_017613,5890
gen.NM_017647,4929
gen.NM_017668,4327
gen.NM_017670,3266
gen.NM_017684,4208
gen.NM_017722,5286
gen.NM_017751,859
gen.NM_017760,2467
gen.NM_017761,91
gen.NM_017768,262
gen.NM_017777,4906
gen.NM_017789,825
gen.NM_017797,5143
gen.NM_017801,1081
gen.NM_017803,4584
gen.NM_017807,4003
gen.NM_017815,3971
gen.NM_017822,3552
gen.NM_017825,165
gen.NM_017827,5413
gen.NM_017829,5939
gen.NM_017847,513
gen.NM_017853,4594
gen.NM_017868,3386
gen.NM_017874,5668
gen.NM_017876,5098
gen.NM_017882,4224
gen.NM_017883,6179
gen.NM_017891,8
gen.NM_017895,5798
gen.NM_017900,22
gen.NM_017901,3810
gen.NM_017910,674
gen.NM_017916,5554
gen.NM_017952,812
gen.NM_017955,4112
gen.NM_017974,1020
gen.NM_018019,4737
gen.NM_018023,1306
gen.NM_018032,4358
gen.NM_018034,1575
gen.NM_018035,5458
gen.NM_018047,1706
gen.NM_018048,3517
gen.NM_018054,4436
gen.NM_018066,116
gen.NM_018070,239
gen.NM_018085,569
gen.NM_018096,4792
gen.NM_018110,4535
gen.NM_018113,3548
gen.NM_018116,420
gen.NM_018122,535
gen.NM_018124,4588
gen.NM_018135,1880
gen.NM_018154,5300
gen.NM_018174,5332
gen.NM_018188,10
gen.NM_018209,5861
gen.NM_018212,587
gen.NM_018217,5740
gen.NM_018238,2437
gen.NM_018242,4747
gen.NM_018250,2510
gen.NM_018253,418
gen.NM_018255,5056
gen.NM_018270,5849
gen.NM_018310,2527
gen.NM_018346,4898
gen.NM_018357,4232
gen.NM_018410,1018
gen.NM_018454,4154
gen.NM_018457,3610
gen.NM_018463,3442
gen.NM_018464,2951
gen.NM_018468,5387
gen.NM_018486,6222
gen.NM_018509,4900
gen.NM_018607,721
gen.NM_018660,2512
gen.NM_018668,4312
gen.NM_018674,973
gen.NM_018686,3513
gen.NM_018912,1734
gen.NM_018913,1736
gen.NM_018914,1738
gen.NM_018915,1740
gen.NM_018916,1742
gen.NM_018917,1744
gen.NM_018918,1746
gen.NM_018919,1748
gen.NM_018920,1750
gen.NM_018921,1752
gen.NM_018922,1754
gen.NM_018923,1756
gen.NM_018924,1758
gen.NM_018925,1760
gen.NM_018926,1762
gen.NM_018927,1764
gen.NM_018928,1766
gen.NM_018929,1768
gen.NM_018947,2208
gen.NM_018948,41
gen.NM_018950,2017
gen.NM_018955,4728
gen.NM_018957,6034
gen.NM_018977,6214
gen.NM_019013,4682
gen.NM_019058,2971
gen.NM_019059,2206
gen.NM_019082,2242
gen.NM_019095,5681
gen.NM_019099,310
gen.NM_019554,371
gen.NM_019606,2333
gen.NM_019609,5663
gen.NM_019619,2916
gen.NM_019848,6321
gen.NM_019852,3988
gen.NM_019887,3839
gen.NM_020037,4895
gen.NM_020038,4893
gen.NM_020132,5908
gen.NM_020134,709
gen.NM_020149,4136
gen.NM_020158,5454
gen.NM_020188,4604
gen.NM_020230,5232
gen.NM_020243,6058
gen.NM_020299,2425
gen.NM_020315,6036
gen.NM_020320,2075
gen.NM_020347,1113
gen.NM_020401,3717
gen.NM_020414,4069
gen.NM_020418,1180
gen.NM_020548,871
gen.NM_020675,896
gen.NM_020677,4340
gen.NM_020701,1248
gen.NM_020990,4172
gen.NM_020992,3017
gen.NM_021019,3646
gen.NM_021029,6244
gen.NM_021079,4883
gen.NM_021095,698
gen.NM_021103,803
gen.NM_021104,3654
gen.NM_021107,5415
gen.NM_021121,948
gen.NM_021126,6029
gen.NM_021129,2964
gen.NM_021130,2238
gen.NM_021141,958
gen.NM_021154,2701
gen.NM_021158,5638
gen.NM_021177,1965
gen.NM_021178,4006
gen.NM_021195,4400
gen.NM_021213,4919
gen.NM_021219,5879
gen.NM_021226,2945
gen.NM_021626,4917
gen.NM_021709,4108
gen.NM_021728,4020
gen.NM_021826,5665
gen.NM_021830,3033
gen.NM_021831,707
gen.NM_021870,1517
gen.NM_021871,1513
gen.NM_021932,3109
gen.NM_021934,3588
gen.NM_021948,394
gen.NM_021953,3444
gen.NM_021966,4079
gen.NM_021999,3908
gen.NM_022003,3369
gen.NM_022039,3039
gen.NM_022044,5973
gen.NM_022048,4216
gen.NM_022105,5857
gen.NM_022137,4042
gen.NM_022141,6101
gen.NM_022158,5016
gen.NM_022170,2288
gen.NM_022171,1145
gen.NM_022362,3029
gen.NM_022369,4246
gen.NM_022371,527
gen.NM_022442,5806
gen.NM_022453,988
gen.NM_022458,2464
gen.NM_022461,1086
gen.NM_022485,1045
gen.NM_022550,1638
gen.NM_022551,1946
gen.NM_022552,717
gen.NM_022566,4296
gen.NM_022727,5961
gen.NM_022744,4468
gen.NM_022747,4084
gen.NM_022748,2226
gen.NM_022752,5474
gen.NM_022758,1926
gen.NM_022770,4539
gen.NM_022778,107
gen.NM_022839,4290
gen.NM_022963,1838
gen.NM_023009,152
gen.NM_023011,3940
gen.NM_023032,3691
gen.NM_023033,3693
gen.NM_023078,2620
gen.NM_023936,4378
gen.NM_023942,2449
gen.NM_024003,6336
gen.NM_024026,3872
gen.NM_024027,645
gen.NM_024029,5250
gen.NM_024031,4458
gen.NM_024033,2427
gen.NM_024040,3047
gen.NM_024045,2957
gen.NM_024048,4470
gen.NM_024067,2186
gen.NM_024068,3643
gen.NM_024070,2335
gen.NM_024089,3935
gen.NM_024098,3218
gen.NM_024099,3236
gen.NM_024104,5323
gen.NM_024111,4148
gen.NM_024294,1924
gen.NM_024297,4672
gen.NM_024299,5865
gen.NM_024319,614
gen.NM_024321,5389
gen.NM_024329,62
gen.NM_024330,379
gen.NM_024333,5186
gen.NM_024339,4396
gen.NM_024407,5120
gen.NM_024507,4406
gen.NM_024516,4502
gen.NM_024537,3938
gen.NM_024567,2508
gen.NM_024571,4350
gen.NM_024572,719
gen.NM_024586,247
gen.NM_024589,4346
gen.NM_024602,206
gen.NM_024603,241
gen.NM_024613,2584
gen.NM_024627,5951
gen.NM_024640,137
gen.NM_024653,2373
gen.NM_024658,3960
gen.NM_024664,183
gen.NM_024668,1724
gen.NM_024671,4454
gen.NM_024691,5636
gen.NM_024709,603
gen.NM_024748,1526
gen.NM_024824,4057
gen.NM_024844,4955
gen.NM_024854,3529
gen.NM_024855,5769
gen.NM_024863,6248
gen.NM_024881,5321
gen.NM_024900,1491
gen.NM_024918,5757
gen.NM_024942,3095
gen.NM_025070,2541
gen.NM_025072,2772
gen.NM_025108,4411
gen.NM_025129,5534
gen.NM_025150,358
gen.NM_025164,3374
gen.NM_025168,1863
gen.NM_025197,4830
gen.NM_025202,1000
gen.NM_025203,678
gen.NM_025204,6109
gen.NM_025205,1414
gen.NM_025207,455
gen.NM_025226,499
gen.NM_025232,2503
gen.NM_025233,4859
gen.NM_025234,4270
gen.NM_025241,5190
gen.NM_025263,2007
gen.NM_030567,1826
gen.NM_030573,5965
gen.NM_030579,4553
gen.NM_030587,196
gen.NM_030593,5411
gen.NM_030775,3432
gen.NM_030782,1545
gen.NM_030815,5719
gen.NM_030819,4573
gen.NM_030877,5763
gen.NM_030900,2232
gen.NM_030920,332
gen.NM_030921,1272
gen.NM_030925,3910
gen.NM_030926,1009
gen.NM_030935,2331
gen.NM_030973,5532
gen.NM_031157,3612
gen.NM_031206,6210
gen.NM_031213,5138
gen.NM_031228,5642
gen.NM_031229,5640
gen.NM_031243,2212
gen.NM_031263,2708
gen.NM_031289,3496
gen.NM_031300,1832
gen.NM_031417,5506
gen.NM_031434,2456
gen.NM_031443,2234
gen.NM_031453,2902
gen.NM_031459,131
gen.NM_031465,3446
gen.NM_031472,3261
gen.NM_031478,4522
gen.NM_031479,3665
gen.NM_031482,1629
gen.NM_031484,3070
gen.NM_031485,5574
gen.NM_031901,336
gen.NM_031925,2304
gen.NM_031942,905
gen.NM_031966,1598
gen.NM_031968,5014
gen.NM_031989,3622
gen.NM_031990,5100
gen.NM_031992,2290
gen.NM_032023,2923
gen.NM_032038,4495
gen.NM_032088,1770
gen.NM_032092,1772
gen.NM_032112,3031
gen.NM_032140,4571
gen.NM_032162,4310
gen.NM_032164,2340
gen.NM_032196,4150
gen.NM_032204,5996
gen.NM_032207,5317
gen.NM_032211,3068
gen.NM_032212,843
gen.NM_032219,1370
gen.NM_032227,6257
gen.NM_032271,4388
gen.NM_032280,1642
gen.NM_032288,1354
gen.NM_032292,412
gen.NM_032299,3395
gen.NM_032313,1437
gen.NM_032322,4771
gen.NM_032323,402
gen.NM_032324,630
gen.NM_032330,4485
gen.NM_032331,1318
gen.NM_032333,2996
gen.NM_032338,3712
gen.NM_032342,2746
gen.NM_032343,1235
gen.NM_032350,2163
gen.NM_032361,1814
gen.NM_032376,4854
gen.NM_032377,5262
gen.NM_032379,3346
gen.NM_032383,1280
gen.NM_032390,875
gen.NM_032402,1776
gen.NM_032403,1774
gen.NM_032486,4444
gen.NM_032527,5869
gen.NM_032565,3914
gen.NM_032626,4440
gen.NM_032627,5345
gen.NM_032635,5393
gen.NM_032636,296
gen.NM_032637,1577
gen.NM_032642,3434
gen.NM_032656,3851
gen.NM_032667,3240
gen.NM_032712,5588
gen.NM_032726,990
gen.NM_032737,5157
gen.NM_032738,503
gen.NM_032747,3061
gen.NM_032750,1174
gen.NM_032753,5173
gen.NM_032756,222
gen.NM_032792,5631
gen.NM_032799,2763
gen.NM_032814,3812
gen.NM_032822,785
gen.NM_032827,810
gen.NM_032864,245
gen.NM_032871,3326
gen.NM_032872,122
gen.NM_032873,3415
gen.NM_032890,606
gen.NM_032904,3794
gen.NM_032905,2893
gen.NM_032907,4248
gen.NM_032928,2860
gen.NM_032929,2081
gen.NM_032933,5037
gen.NM_032951,2284
gen.NM_032953,2286
gen.NM_032958,2376
gen.NM_032989,3258
gen.NM_032997,2949
gen.NM_032999,2295
gen.NM_033008,1176
gen.NM_033010,1178
gen.NM_033011,2538
gen.NM_033022,2978
gen.NM_033046,796
gen.NM_033070,5937
gen.NM_033161,2828
gen.NM_033197,5729
gen.NM_033219,2730
gen.NM_033251,4635
gen.NM_033296,1404
gen.NM_033301,2635
gen.NM_033316,1348
gen.NM_033363,5417
gen.NM_033410,4456
gen.NM_033415,5355
gen.NM_033416,878
gen.NM_033421,5787
gen.NM_033440,60
gen.NM_033534,15
gen.NM_033544,4315
gen.NM_033551,1785
gen.NM_052837,426
gen.NM_052848,5451
gen.NM_052859,1157
gen.NM_052862,488
gen.NM_052881,5656
gen.NM_052886,2602
gen.NM_052936,6251
gen.NM_052963,2616
gen.NM_052984,3685
gen.NM_053043,2462
gen.NM_053056,3311
gen.NM_053275,3829
gen.NM_054012,2822
gen.NM_054013,1848
gen.NM_054014,5650
gen.NM_054016,95
gen.NM_057089,2363
gen.NM_057161,1890
gen.NM_057169,3790
gen.NM_057174,3188
gen.NM_057182,5376
gen.NM_058164,5230
gen.NM_058179,2703
gen.NM_058192,4366
gen.NM_058193,3422
gen.NM_058195,2653
gen.NM_058196,2657
gen.NM_058199,2836
gen.NM_078467,1912
gen.NM_079423,3648
gen.NM_079425,3650
gen.NM_080424,1016
gen.NM_080425,5828
gen.NM_080426,5832
gen.NM_080491,3342
gen.NM_080592,696
gen.NM_080594,4394
gen.NM_080598,1984
gen.NM_080648,3999
gen.NM_080649,4001
gen.NM_080670,1726
gen.NM_080686,1981
gen.NM_080687,3942
gen.NM_080702,1977
gen.NM_080703,1975
gen.NM_080796,5855
gen.NM_080797,5859
gen.NM_080820,5693
gen.NM_080822,4654
gen.NM_106552,670
gen.NM_130398,639
gen.NM_130442,2260
gen.NM_130468,4143
gen.NM_130898,434
gen.NM_133330,1376
gen.NM_133332,1380
gen.NM_133373,4885
gen.NM_133375,4222
gen.NM_133436,2357
gen.NM_133480,1051
gen.NM_133481,1053
gen.NM_133483,3676
gen.NM_133503,3742
gen.NM_133504,3744
gen.NM_133505,3746
gen.NM_133506,3750
gen.NM_133507,3748
gen.NM_133627,4786
gen.NM_133629,4790
gen.NM_133630,4788
gen.NM_133637,798
gen.NM_133645,2066
gen.NM_134269,6009
gen.NM_134323,3616
gen.NM_134324,3618
gen.NM_134440,5358
gen.NM_138385,1372
gen.NM_138391,545
gen.NM_138427,4739
gen.NM_138434,2451
gen.NM_138443,5060
gen.NM_138483,1037
gen.NM_138578,5713
gen.NM_138614,1125
gen.NM_138699,1406
gen.NM_138801,727
gen.NM_138924,5124
gen.XM_001289,524
gen.XM_001299,33
gen.XM_001389,1453
gen.XM_001468,342
gen.XM_001472,250
gen.XM_001482,3658
gen.XM_001589,24
gen.XM_001616,101
gen.XM_001640,126
gen.XM_001807,135
gen.XM_001812,134
gen.XM_001826,78
gen.XM_001897,486
gen.XM_001914,567
gen.XM_001916,568
gen.XM_001958,599
gen.XM_002068,523
gen.XM_002105,141
gen.XM_002114,113
gen.XM_002217,845
gen.XM_002255,1361
gen.XM_002435,700
gen.XM_002447,877
gen.XM_002480,680
gen.XM_002540,1006
gen.XM_002611,823
gen.XM_002636,964
gen.XM_002647,770
gen.XM_002669,946
gen.XM_002674,776
gen.XM_002704,853
gen.XM_002727,788
gen.XM_002739,779
gen.XM_002742,1036
gen.XM_002828,1143
gen.XM_002854,1187
gen.XM_002855,1186
gen.XM_002859,1274
gen.XM_002899,1127
gen.XM_003213,1162
gen.XM_003222,1119
gen.XM_003245,1136
gen.XM_003305,1451
gen.XM_003435,1432
gen.XM_003477,1530
gen.XM_003511,1448
gen.XM_003555,1500
gen.XM_003611,2083
gen.XM_003716,1811
gen.XM_003771,1644
gen.XM_003789,1712
gen.XM_003825,1540
gen.XM_003830,1666
gen.XM_003841,1699
gen.XM_003869,1572
gen.XM_003896,1581
gen.XM_003937,1710
gen.XM_004009,1565
gen.XM_004098,3704
gen.XM_004151,2065
gen.XM_004256,2114
gen.XM_004297,2113
gen.XM_004330,3194
gen.XM_004379,2122
gen.XM_004383,2130
gen.XM_004526,2110
gen.XM_004627,2402
gen.XM_004901,2292
gen.XM_005060,2605
gen.XM_005086,1042
gen.XM_005100,2908
gen.XM_005180,1332
gen.XM_005305,2485
gen.XM_005348,2755
gen.XM_005365,2760
gen.XM_005490,2707
gen.XM_005525,2727
gen.XM_005543,2666
gen.XM_005675,3103
gen.XM_005698,3053
gen.XM_005724,2878
gen.XM_005938,3058
gen.XM_005969,3088
gen.XM_006139,3127
gen.XM_006170,3201
gen.XM_006212,3167
gen.XM_006290,98
gen.XM_006297,3196
gen.XM_006424,3151
gen.XM_006432,3371
gen.XM_006464,3355
gen.XM_006467,3399
gen.XM_006475,3135
gen.XM_006483,3136
gen.XM_006529,3281
gen.XM_006533,3270
gen.XM_006566,3849
gen.XM_006578,3736
gen.XM_006589,3766
gen.XM_006595,3835
gen.XM_006694,3535
gen.XM_006710,3626
gen.XM_006748,3536
gen.XM_006826,3559
gen.XM_006887,3765
gen.XM_006925,3485
gen.XM_006936,3483
gen.XM_006937,5074
gen.XM_006947,3482
gen.XM_006958,3475
gen.XM_007002,3797
gen.XM_007003,3796
gen.XM_007199,3923
gen.XM_007254,4097
gen.XM_007272,4081
gen.XM_007288,3968
gen.XM_007293,3967
gen.XM_007315,3958
gen.XM_007316,3957
gen.XM_007324,4027
gen.XM_007328,4024
gen.XM_007441,4045
gen.XM_007483,4072
gen.XM_007488,4005
gen.XM_007491,3996
gen.XM_007531,4167
gen.XM_007545,4156
gen.XM_007623,4221
gen.XM_007651,4189
gen.XM_007751,4129
gen.XM_007963,4474
gen.XM_007988,4430
gen.XM_008064,4509
gen.XM_008065,4497
gen.XM_008106,4463
gen.XM_008126,4353
gen.XM_008150,4800
gen.XM_008231,4694
gen.XM_008253,4926
gen.XM_008323,4750
gen.XM_008334,4671
gen.XM_008351,4856
gen.XM_008401,4867
gen.XM_008402,4869
gen.XM_008432,4902
gen.XM_008441,4686
gen.XM_008459,4915
gen.XM_008462,4777
gen.XM_008486,4760
gen.XM_008509,4658
gen.XM_008538,4684
gen.XM_008557,4650
gen.XM_008579,4809
gen.XM_008679,4693
gen.XM_008695,5089
gen.XM_008723,5054
gen.XM_008812,5083
gen.XM_008830,5597
gen.XM_008851,5522
gen.XM_008854,5325
gen.XM_008860,5485
gen.XM_008878,5472
gen.XM_008887,5243
gen.XM_008912,5453
gen.XM_008985,5531
gen.XM_009010,5205
gen.XM_009036,5486
gen.XM_009063,5274
gen.XM_009082,5256
gen.XM_009125,5484
gen.XM_009126,5496
gen.XM_009149,5406
gen.XM_009180,5378
gen.XM_009203,5443
gen.XM_009222,5165
gen.XM_009277,5113
gen.XM_009279,5110
gen.XM_009293,5338
gen.XM_009303,5310
gen.XM_009330,5357
gen.XM_009338,5384
gen.XM_009436,5705
gen.XM_009450,5728
gen.XM_009501,5754
gen.XM_009549,5816
gen.XM_009622,5647
gen.XM_009642,5759
gen.XM_009671,5823
gen.XM_009672,5821
gen.XM_009686,5762
gen.XM_009805,5919
gen.XM_009947,6022
gen.XM_009967,6031
gen.XM_009973,6042
gen.XM_010000,6063
gen.XM_010002,6064
gen.XM_010024,6087
gen.XM_010029,6094
gen.XM_010040,6103
gen.XM_010055,6108
gen.XM_010117,6269
gen.XM_010141,6216
gen.XM_010156,5266
gen.XM_010178,6310
gen.XM_010272,6132
gen.XM_010362,6274
gen.XM_010378,6169
gen.XM_010436,6280
gen.XM_010494,3429
gen.XM_010615,253
gen.XM_010636,451
gen.XM_010664,133
gen.XM_010682,581
gen.XM_010712,182
gen.XM_010732,593
gen.XM_010778,925
gen.XM_010852,938
gen.XM_010858,1004
gen.XM_010866,992
gen.XM_010881,771
gen.XM_010886,755
gen.XM_010938,4641
gen.XM_010941,1433
gen.XM_010953,1130
gen.XM_010978,1290
gen.XM_011074,1320
gen.XM_011089,5076
gen.XM_011117,2059
gen.XM_011118,4941
gen.XM_011129,1423
gen.XM_011160,1365
gen.XM_011548,2411
gen.XM_011618,2400
gen.XM_011629,2533
gen.XM_011642,2586
gen.XM_011650,66
gen.XM_011657,2592
gen.XM_011749,2798
gen.XM_011752,2786
gen.XM_011769,2562
gen.XM_011778,2832
gen.XM_011988,3260
gen.XM_012124,3836
gen.XM_012145,3761
gen.XM_012159,3494
gen.XM_012162,3598
gen.XM_012179,5337
gen.XM_012182,3638
gen.XM_012184,3861
gen.XM_012219,3759
gen.XM_012272,3543
gen.XM_012284,2395
gen.XM_012376,3990
gen.XM_012377,3983
gen.XM_012398,4133
gen.XM_012418,4199
gen.XM_012462,4322
gen.XM_012487,4555
gen.XM_012549,4734
gen.XM_012569,4461
gen.XM_012609,4945
gen.XM_012615,4744
gen.XM_012634,4950
gen.XM_012638,3874
gen.XM_012642,4849
gen.XM_012651,4916
gen.XM_012676,4675
gen.XM_012741,5031
gen.XM_012798,5212
gen.XM_012812,5370
gen.XM_012860,5439
gen.XM_012862,5195
gen.XM_012913,5114
gen.XM_012931,5768
gen.XM_012970,5700
gen.XM_013010,6066
gen.XM_013015,6089
gen.XM_013029,6118
gen.XM_013042,6207
gen.XM_013060,6196
gen.XM_013086,6145
gen.XM_013112,2530
gen.XM_013127,2577
gen.XM_015234,75
gen.XM_015241,5088
gen.XM_015243,3148
gen.XM_015258,2244
gen.XM_015366,4239
gen.XM_015434,547
gen.XM_015462,1208
gen.XM_015468,3596
gen.XM_015476,3585
gen.XM_015481,3580
gen.XM_015516,6206
gen.XM_015563,1525
gen.XM_015652,2937
gen.XM_015697,5264
gen.XM_015700,4478
gen.XM_015705,3214
gen.XM_015717,257
gen.XM_015755,5046
gen.XM_015769,5369
gen.XM_015835,4311
gen.XM_015840,3921
gen.XM_015842,3932
gen.XM_015920,909
gen.XM_015922,911
gen.XM_016047,2604
gen.XM_016076,4237
gen.XM_016093,2992
gen.XM_016113,2712
gen.XM_016125,6275
gen.XM_016139,3170
gen.XM_016164,276
gen.XM_016170,1554
gen.XM_016199,600
gen.XM_016288,880
gen.XM_016308,2726
gen.XM_016334,1294
gen.XM_016345,1799
gen.XM_016351,3924
gen.XM_016378,5364
gen.XM_016382,5036
gen.XM_016410,5438
gen.XM_016480,326
gen.XM_016486,4071
gen.XM_016487,4068
gen.XM_016605,3708
gen.XM_016625,773
gen.XM_016640,3538
gen.XM_016674,1652
gen.XM_016700,2433
gen.XM_016713,4165
gen.XM_016733,2256
gen.XM_016843,766
gen.XM_016857,1941
gen.XM_016871,5180
gen.XM_016985,4213
gen.XM_017080,3436
gen.XM_017096,4644
gen.XM_017204,5240
gen.XM_017234,4712
gen.XM_017240,4135
gen.XM_017315,67
gen.XM_017356,1291
gen.XM_017364,1105
gen.XM_017369,3394
gen.XM_017432,3895
gen.XM_017442,2313
gen.XM_017474,1679
gen.XM_017483,2280
gen.XM_017508,3710
gen.XM_017517,2080
gen.XM_017578,4980
gen.XM_017591,1701
gen.XM_017641,1544
gen.XM_017698,861
gen.XM_017816,2581
gen.XM_017831,2119
gen.XM_017846,109
gen.XM_017857,1640
gen.XM_017914,3953
gen.XM_017925,1476
gen.XM_017930,6284
gen.XM_017931,2659
gen.XM_017971,4319
gen.XM_017984,4338
gen.XM_017996,2711
gen.XM_018006,2710
gen.XM_018019,6157
gen.XM_018039,784
gen.XM_018041,642
gen.XM_018054,4123
gen.XM_018088,4472
gen.XM_018108,6313
gen.XM_018109,6315
gen.XM_018136,161
gen.XM_018142,6232
gen.XM_018149,1264
gen.XM_018167,3015
gen.XM_018182,2098
gen.XM_018205,64
gen.XM_018241,6161
gen.XM_018279,3057
gen.XM_018287,2595
gen.XM_018301,763
gen.XM_018332,314
gen.XM_018359,2281
gen.XM_018399,3918
gen.XM_018432,4331
gen.XM_018473,1658
gen.XM_018515,5354
gen.XM_018523,1359
gen.XM_018534,4840
gen.XM_018539,6014
gen.XM_018540,841
gen.XM_026944,2787
gen.XM_026951,2771
gen.XM_026968,2769
gen.XM_026985,2766
gen.XM_026987,2765
gen.XM_027102,3802
gen.XM_027143,6106
gen.XM_027161,1220
gen.XM_027214,2385
gen.XM_027309,4329
gen.XM_027313,226
gen.XM_027365,4334
gen.XM_027412,4368
gen.XM_027440,2505
gen.XM_027558,4352
gen.XM_027651,2490
gen.XM_027679,2488
gen.XM_027825,4661
gen.XM_027904,5548
gen.XM_027916,76
gen.XM_027952,6353
gen.XM_027963,936
gen.XM_027964,1619
gen.XM_027983,213
gen.XM_028034,940
gen.XM_028064,5119
gen.XM_028067,5117
gen.XM_028151,4562
gen.XM_028192,3117
gen.XM_028263,5488
gen.XM_028267,5491
gen.XM_028322,4075
gen.XM_028347,4074
gen.XM_028358,4073
gen.XM_028398,4667
gen.XM_028417,4678
gen.XM_028643,3624
gen.XM_028662,3561
gen.XM_028666,5383
gen.XM_028672,5382
gen.XM_028744,5025
gen.XM_028760,3554
gen.XM_028783,5851
gen.XM_028806,5765
gen.XM_028810,5766
gen.XM_028834,5863
gen.XM_028848,4390
gen.XM_028918,5867
gen.XM_028966,5871
gen.XM_029031,169
gen.XM_029096,1539
gen.XM_029104,1314
gen.XM_029132,1313
gen.XM_029136,1310
gen.XM_029168,2841
gen.XM_029187,6194
gen.XM_029228,2069
gen.XM_029288,4067
gen.XM_029369,1198
gen.XM_029438,4656
gen.XM_029450,5404
gen.XM_029455,5403
gen.XM_029461,6282
gen.XM_029567,2609
gen.XM_029631,3602
gen.XM_029728,3595
gen.XM_029746,2128
gen.XM_029805,3507
gen.XM_029810,5776
gen.XM_029822,5778
gen.XM_029842,176
gen.XM_029844,145
gen.XM_030044,5796
gen.XM_030203,1028
gen.XM_030268,2543
gen.XM_030274,2544
gen.XM_030326,3187
gen.XM_030373,6233
gen.XM_030417,1112
gen.XM_030423,154
gen.XM_030447,3065
gen.XM_030470,68
gen.XM_030485,5159
gen.XM_030529,862
gen.XM_030582,883
gen.XM_030621,5818
gen.XM_030699,5834
gen.XM_030714,5145
gen.XM_030720,5137
gen.XM_030721,5135
gen.XM_030771,1821
gen.XM_030777,1823
gen.XM_030782,1824
gen.XM_030812,1256
gen.XM_030834,952
gen.XM_030895,5465
gen.XM_030901,5456
gen.XM_030914,5450
gen.XM_030920,40
gen.XM_031025,4032
gen.XM_031074,4039
gen.XM_031251,5307
gen.XM_031263,5305
gen.XM_031273,5303
gen.XM_031276,5302
gen.XM_031292,4295
gen.XM_031320,1445
gen.XM_031345,5292
gen.XM_031354,4292
gen.XM_031404,4285
gen.XM_031415,4767
gen.XM_031427,4769
gen.XM_031466,4765
gen.XM_031515,4147
gen.XM_031519,731
gen.XM_031527,733
gen.XM_031536,4758
gen.XM_031554,4145
gen.XM_031586,782
gen.XM_031586,783
gen.XM_031596,780
gen.XM_031617,4138
gen.XM_031626,738
gen.XM_031718,4159
gen.XM_031807,3491
gen.XM_031857,5184
gen.XM_031866,3041
gen.XM_031890,3044
gen.XM_031917,5176
gen.XM_031944,5066
gen.XM_031949,3049
gen.XM_031992,3059
gen.XM_032020,5281
gen.XM_032121,2455
gen.XM_032201,4836
gen.XM_032216,2454
gen.XM_032269,1221
gen.XM_032285,5399
gen.XM_032391,216
gen.XM_032403,4180
gen.XM_032443,3930
gen.XM_032476,2976
gen.XM_032520,2970
gen.XM_032553,1626
gen.XM_032588,3457
gen.XM_032614,3462
gen.XM_032710,5247
gen.XM_032719,5248
gen.XM_032724,5252
gen.XM_032759,1700
gen.XM_032766,4864
gen.XM_032774,5257
gen.XM_032782,5261
gen.XM_032813,4863
gen.XM_032817,4861
gen.XM_032852,4857
gen.XM_032895,1590
gen.XM_032902,1588
gen.XM_032930,6189
gen.XM_032944,2470
gen.XM_032996,5943
gen.XM_033015,5902
gen.XM_033016,5903
gen.XM_033090,5946
gen.XM_033147,6241
gen.XM_033227,3450
gen.XM_033232,6351
gen.XM_033251,3959
gen.XM_033263,3472
gen.XM_033294,1123
gen.XM_033337,3964
gen.XM_033355,2819
gen.XM_033359,2818
gen.XM_033360,2817
gen.XM_033361,2815
gen.XM_033362,2811
gen.XM_033380,2809
gen.XM_033385,2808
gen.XM_033391,3969
gen.XM_033424,2774
gen.XM_033435,3975
gen.XM_033445,3980
gen.XM_033457,2777
gen.XM_033460,2778
gen.XM_033553,3991
gen.XM_033595,3994
gen.XM_033654,79
gen.XM_033683,77
gen.XM_033689,4646
gen.XM_033714,4645
gen.XM_033813,5960
gen.XM_033862,6173
gen.XM_033876,2383
gen.XM_033878,6172
gen.XM_033884,6170
gen.XM_033910,2134
gen.XM_033912,2132
gen.XM_033922,4606
gen.XM_034000,501
gen.XM_034082,454
gen.XM_034321,1502
gen.XM_034375,4460
gen.XM_034377,5623
gen.XM_034431,3185
gen.XM_034586,4376
gen.XM_034590,4380
gen.XM_034640,2638
gen.XM_034662,319
gen.XM_034671,318
gen.XM_034710,1466
gen.XM_034713,1468
gen.XM_034744,1655
gen.XM_034862,1675
gen.XM_034890,4184
gen.XM_034897,4256
gen.XM_034935,6201
gen.XM_034952,857
gen.XM_034953,4116
gen.XM_035014,4119
gen.XM_035103,2824
gen.XM_035107,2439
gen.XM_035109,2825
gen.XM_035220,800
gen.XM_035368,2626
gen.XM_035370,2631
gen.XM_035373,2629
gen.XM_035465,6123
gen.XM_035485,3571
gen.XM_035490,3564
gen.XM_035497,3562
gen.XM_035572,1392
gen.XM_035625,5197
gen.XM_035627,5196
gen.XM_035636,5194
gen.XM_035638,5192
gen.XM_035640,5034
gen.XM_035662,2483
gen.XM_035680,2482
gen.XM_035824,1402
gen.XM_035919,5612
gen.XM_035986,1456
gen.XM_035999,5907
gen.XM_036002,1440
gen.XM_036011,5910
gen.XM_036042,5913
gen.XM_036087,5917
gen.XM_036104,4965
gen.XM_036107,5923
gen.XM_036115,4971
gen.XM_036118,1262
gen.XM_036175,5924
gen.XM_036299,155
gen.XM_036339,3178
gen.XM_036413,2469
gen.XM_036450,664
gen.XM_036462,4827
gen.XM_036465,4825
gen.XM_036500,573
gen.XM_036507,575
gen.XM_036528,4410
gen.XM_036556,566
gen.XM_036593,2939
gen.XM_036659,4707
gen.XM_036680,4342
gen.XM_036727,4134
gen.XM_036744,433
gen.XM_036755,5927
gen.XM_036785,4982
gen.XM_036829,442
gen.XM_036845,450
gen.XM_036934,448
gen.XM_036937,5969
gen.XM_036938,1197
gen.XM_037002,1668
gen.XM_037056,2107
gen.XM_037101,873
gen.XM_037108,831
gen.XM_037147,3212
gen.XM_037173,3202
gen.XM_037195,4988
gen.XM_037196,4987
gen.XM_037202,5840
gen.XM_037206,5842
gen.XM_037217,5846
gen.XM_037260,1608
gen.XM_037329,591
gen.XM_037377,1300
gen.XM_037381,1299
gen.XM_037423,1163
gen.XM_037468,6114
gen.XM_037474,6116
gen.XM_037565,5106
gen.XM_037572,5109
gen.XM_037600,1304
gen.XM_037657,2608
gen.XM_037662,5372
gen.XM_037682,5977
gen.XM_037741,2276
gen.XM_037778,4244
gen.XM_037797,5981
gen.XM_037808,3263
gen.XM_037875,2045
gen.XM_037945,5993
gen.XM_037971,4897
gen.XM_038030,2855
gen.XM_038049,2864
gen.XM_038063,2866
gen.XM_038098,5343
gen.XM_038146,5339
gen.XM_038221,1695
gen.XM_038243,1341
gen.XM_038308,3737
gen.XM_038371,3902
gen.XM_038391,2757
gen.XM_038424,5018
gen.XM_038536,2909
gen.XM_038576,734
gen.XM_038584,6019
gen.XM_038659,3533
gen.XM_038791,3841
gen.XM_038852,244
gen.XM_038872,5062
gen.XM_038911,237
gen.XM_038946,1840
gen.XM_039165,1413
gen.XM_039173,1416
gen.XM_039176,1417
gen.XM_039225,4125
gen.XM_039236,6047
gen.XM_039248,6051
gen.XM_039306,4551
gen.XM_039339,6060
gen.XM_039372,6065
gen.XM_039395,3732
gen.XM_039474,4794
gen.XM_039654,2646
gen.XM_039702,4200
gen.XM_039712,716
gen.XM_039721,321
gen.XM_039723,5140
gen.XM_039796,1292
gen.XM_039805,1258
gen.XM_039908,5598
gen.XM_039910,4721
gen.XM_039921,4732
gen.XM_039952,1213
gen.XM_039975,1783
gen.XM_040009,377
gen.XM_040066,6088
gen.XM_040095,6091
gen.XM_040221,3707
gen.XM_040267,2879
gen.XM_040272,2876
gen.XM_040321,1524
gen.XM_040498,2417
gen.XM_040623,2074
gen.XM_040644,3734
gen.XM_040709,315
gen.XM_040752,1493
gen.XM_040853,2218
gen.XM_040898,4100
gen.XM_040942,4094
gen.XM_040952,4090
gen.XM_041014,4086
gen.XM_041020,2697
gen.XM_041059,1670
gen.XM_041100,3503
gen.XM_041209,3925
gen.XM_041211,1161
gen.XM_041221,1410
gen.XM_041235,4008
gen.XM_041248,6111
gen.XM_041473,3928
gen.XM_041484,3944
gen.XM_041507,1147
gen.XM_041583,4957
gen.XM_041678,5027
gen.XM_041694,1614
gen.XM_041712,1592
gen.XM_041872,5090
gen.XM_041879,353
gen.XM_041884,354
gen.XM_041921,6304
gen.XM_041964,4680
gen.XM_042018,5095
gen.XM_042025,1600
gen.XM_042153,6348
gen.XM_042155,6346
gen.XM_042168,1286
gen.XM_042301,1474
gen.XM_042326,1032
gen.XM_042422,2145
gen.XM_042473,2148
gen.XM_042618,1229
gen.XM_042621,4596
gen.XM_042658,2561
gen.XM_042695,1364
gen.XM_042698,4710
gen.XM_042765,5701
gen.XM_042781,2434
gen.XM_042788,2744
gen.XM_042841,1072
gen.XM_042852,3339
gen.XM_042860,1070
gen.XM_042963,6295
gen.XM_042967,537
gen.XM_042968,6297
gen.XM_043047,4577
gen.XM_043173,866
gen.XM_043220,3111
gen.XM_043340,1805
gen.XM_043388,1808
gen.XM_043589,2998
gen.XM_043605,2999
gen.XM_043614,6099
gen.XM_043643,6250
gen.XM_043771,1568
gen.XM_044075,416
gen.XM_044077,391
gen.XM_044127,398
gen.XM_044128,408
gen.XM_044166,406
gen.XM_044172,411
gen.XM_044334,3859
gen.XM_044354,2968
gen.XM_044367,4938
gen.XM_044372,4943
gen.XM_044376,4935
gen.XM_044394,4927
gen.XM_044426,4924
gen.XM_044523,4304
gen.XM_044533,4307
gen.XM_044565,4269
gen.XM_044569,4272
gen.XM_044593,4278
gen.XM_044608,5213
gen.XM_044619,5210
gen.XM_044627,2563
gen.XM_044866,2139
gen.XM_044914,5658
gen.XM_044915,5660
gen.XM_044932,3129
gen.XM_044957,3131
gen.XM_045010,3821
gen.XM_045044,4749
gen.XM_045104,4989
gen.XM_045140,2973
gen.XM_045151,5226
gen.XM_045170,928
gen.XM_045183,4651
gen.XM_045187,3833
gen.XM_045283,757
gen.XM_045290,1214
gen.XM_045296,2759
gen.XM_045401,2403
gen.XM_045418,5667
gen.XM_045451,5671
gen.XM_045460,5674
gen.XM_045499,3276
gen.XM_045525,3278
gen.XM_045535,4751
gen.XM_045551,4752
gen.XM_045581,4996
gen.XM_045602,3856
gen.XM_045612,3273
gen.XM_045613,3271
gen.XM_045642,3269
gen.XM_045667,3074
gen.XM_045681,4287
gen.XM_045750,3157
gen.XM_045802,3826
gen.XM_045856,2407
gen.XM_045901,4852
gen.XM_045952,2413
gen.XM_045963,3834
gen.XM_046001,2414
gen.XM_046035,4453
gen.XM_046041,3726
gen.XM_046057,1443
gen.XM_046090,5423
gen.XM_046160,5708
gen.XM_046179,5710
gen.XM_046313,5544
gen.XM_046349,187
gen.XM_046401,1085
gen.XM_046419,5578
gen.XM_046450,201
gen.XM_046464,522
gen.XM_046472,5004
gen.XM_046481,4999
gen.XM_046520,5689
gen.XM_046551,212
gen.XM_046557,208
gen.XM_046565,204
gen.XM_046642,3951
gen.XM_046648,3950
gen.XM_046651,3949
gen.XM_046743,3035
gen.XM_046765,5020
gen.XM_046767,5022
gen.XM_046769,5021
gen.XM_046822,5150
gen.XM_046836,2722
gen.XM_046863,2720
gen.XM_046918,112
gen.XM_046932,4958
gen.XM_046934,5160
gen.XM_047007,5723
gen.XM_047011,5725
gen.XM_047018,5727
gen.XM_047024,6177
gen.XM_047032,6176
gen.XM_047083,2521
gen.XM_047175,690
gen.XM_047374,5446
gen.XM_047376,5445
gen.XM_047409,5444
gen.XM_047436,4624
gen.XM_047477,1429
gen.XM_047479,495
gen.XM_047499,610
gen.XM_047525,4632
gen.XM_047545,616
gen.XM_047561,1137
gen.XM_047584,5131
gen.XM_047600,5132
gen.XM_047964,1798
gen.XM_048088,753
gen.XM_048119,4344
gen.XM_048258,5385
gen.XM_048286,3255
gen.XM_048351,5218
gen.XM_048364,5219
gen.XM_048404,6329
gen.XM_048410,6328
gen.XM_048420,6325
gen.XM_048471,5082
gen.XM_048479,2679
gen.XM_048518,2684
gen.XM_048539,2684
gen.XM_048603,3674
gen.XM_048654,4829
gen.XM_048690,1007
gen.XM_048780,57
gen.XM_048859,2881
gen.XM_048905,6306
gen.XM_048943,3640
gen.XM_048957,3931
gen.XM_048991,3642
gen.XM_049048,3652
gen.XM_049108,820
gen.XM_049113,822
gen.XM_049116,818
gen.XM_049141,3586
gen.XM_049148,3581
gen.XM_049150,3659
gen.XM_049197,3161
gen.XM_049201,3772
gen.XM_049211,3771
gen.XM_049226,2623
gen.XM_049237,5391
gen.XM_049247,2618
gen.XM_049282,5223
gen.XM_049310,139
gen.XM_049337,6320
gen.XM_049354,4275
gen.XM_049372,4317
gen.XM_049421,2637
gen.XM_049502,5236
gen.XM_049561,5239
gen.XM_049663,3493
gen.XM_049680,476
gen.XM_049690,483
gen.XM_049742,14
gen.XM_049795,3082
gen.XM_049899,2121
gen.XM_049904,3937
gen.XM_049920,5482
gen.XM_049931,4995
gen.XM_049934,4994
gen.XM_049937,4818
gen.XM_050074,3528
gen.XM_050101,4773
gen.XM_050159,4880
gen.XM_050194,4462
gen.XM_050200,1487
gen.XM_050215,2525
gen.XM_050236,5602
gen.XM_050265,2278
gen.XM_050278,4103
gen.XM_050293,2487
gen.XM_050403,6192
gen.XM_050430,2389
gen.XM_050435,5227
gen.XM_050506,2583
gen.XM_050534,4348
gen.XM_050552,1234
gen.XM_050589,5603
gen.XM_050638,979
gen.XM_050660,5330
gen.XM_050731,2571
gen.XM_050891,984
gen.XM_050962,975
gen.XM_050964,4220
gen.XM_051219,4479
gen.XM_051264,1237
gen.XM_051298,2612
gen.XM_051364,5290
gen.XM_051430,3398
gen.XM_051435,3358
gen.XM_051463,4230
gen.XM_051471,6238
gen.XM_051476,6237
gen.XM_051489,3367
gen.XM_051518,1131
gen.XM_051556,6
gen.XM_051586,5092
gen.XM_051712,4025
gen.XM_051716,3373
gen.XM_051763,4727
gen.XM_051778,4600
gen.XM_051860,4298
gen.XM_051877,515
gen.XM_052113,3378
gen.XM_052310,1060
gen.XM_052313,1535
gen.XM_052336,1477
gen.XM_052460,3714
gen.XM_052474,3719
gen.XM_052530,1424
gen.XM_052542,3755
gen.XM_052626,1398
gen.XM_052635,5166
gen.XM_052641,3769
gen.XM_052661,5168
gen.XM_052721,2056
gen.XM_052725,2784
gen.XM_052786,3153
gen.XM_052862,3404
gen.XM_052893,3825
gen.XM_052974,608
gen.XM_052989,817
gen.XM_053074,5430
gen.XM_053122,1363
gen.XM_053164,3641
gen.XM_053183,58
gen.XM_053206,2875
gen.XM_053245,400
gen.XM_053323,1078
gen.XM_053585,4252
gen.XM_053633,544
gen.XM_053712,1074
gen.XM_053717,4663
gen.XM_053787,3283
gen.XM_053796,3288
gen.XM_053952,3722
gen.XM_053955,1859
gen.XM_054038,4832
gen.XM_054098,6183
gen.XM_054221,6155
gen.XM_054344,4973
gen.XM_054474,2933
gen.XM_054475,2935
gen.XM_054520,1047
gen.XM_054566,5926
gen.XM_054706,2146
gen.XM_054752,2849
gen.XM_054763,2852
gen.XM_054856,3193
gen.XM_054868,228
gen.XM_054900,4309
gen.XM_054978,295
gen.XM_055013,3853
gen.XM_055061,4826
gen.XM_055132,4514
gen.XM_055195,4427
gen.XM_055199,4942
gen.XM_055230,5336
gen.XM_055254,954
gen.XM_055369,3397
gen.XM_055481,251
gen.XM_055551,1461
gen.XM_055573,3086
gen.XM_055641,2064
gen.XM_055658,5592
gen.XM_055686,5163
gen.XM_055771,4505
gen.XM_055859,5483
gen.XM_055880,583
gen.XM_055993,5646
gen.XM_056035,5678
gen.XM_056082,4648
gen.XM_056260,4438
gen.XM_056286,5582
gen.XM_056315,1723
gen.XM_056317,4077
gen.XM_056346,3645
gen.XM_056353,3662
gen.XM_056421,5175
gen.XM_056481,3545
gen.XM_056602,5408
gen.XM_056681,3700
gen.XM_056730,4775
gen.XM_056884,618
gen.XM_056923,521
gen.XM_056957,1471
gen.XM_056963,1793
gen.XM_056970,628
gen.XM_056996,3798
gen.XM_057020,4257
gen.XM_057074,5260
gen.XM_057150,4619
gen.XM_057236,5756
gen.XM_057374,5793
gen.XM_057492,1548
gen.XM_057664,740
gen.XM_057780,2557
gen.XM_057994,1541
gen.XM_058039,1934
gen.XM_058098,986
gen.XM_058116,4526
gen.XM_058125,5635
gen.XM_058210,4018
gen.XM_058232,5225
gen.XM_058240,102
gen.XM_058247,466
gen.XM_058266,2144
gen.XM_058267,1278
gen.XM_058343,3020
gen.XM_058361,3078
gen.XM_058405,552
gen.XM_058406,3084
gen.XM_058414,3159
gen.XM_058450,3352
gen.XM_058505,3125
gen.XM_058528,3671
gen.XM_058556,3773
gen.XM_058567,3504
gen.XM_058574,3454
gen.XM_058602,3022
gen.XM_058611,3926
gen.XM_058618,4091
gen.XM_058636,4118
gen.XM_058646,3986
gen.XM_058647,3978
gen.XM_058677,4061
gen.XM_058684,4186
gen.XM_058699,4250
gen.XM_058702,294
gen.XM_058739,4621
gen.XM_058745,4543
gen.XM_058784,4404
gen.XM_058796,4337
gen.XM_058830,4803
gen.XM_058867,4755
gen.XM_058900,4730
gen.XM_058918,5949
gen.XM_058927,1441
gen.XM_058949,5463
gen.XM_058967,5295
gen.XM_058968,2619
gen.XM_058977,3920
gen.XM_058987,5570
gen.XM_058990,5584
gen.XM_058991,5552
gen.XM_059045,5419
gen.XM_059052,5447
gen.XM_059066,114
gen.XM_059067,120
gen.XM_059088,130
gen.XM_059094,465
gen.XM_059117,103
gen.XM_059120,562
gen.XM_059133,224
gen.XM_059171,171
gen.XM_059180,256
gen.XM_059191,492
gen.XM_059201,1
gen.XM_059210,330
gen.XM_059214,185
gen.XM_059230,55
gen.XM_059268,5675
gen.XM_059321,5607
gen.XM_059335,6013
gen.XM_059351,920
gen.XM_059368,653
gen.XM_059372,1029
gen.XM_059422,968
gen.XM_059461,971
gen.XM_059465,907
gen.XM_059516,1266
gen.XM_059557,1068
gen.XM_059561,1059
gen.XM_059583,1252
gen.XM_059593,1434
gen.XM_059623,1519
gen.XM_059628,1442
gen.XM_059633,1469
gen.XM_059637,2804
gen.XM_059653,1596
gen.XM_059669,1617
gen.XM_059709,1604
gen.XM_059720,2914
gen.XM_059741,2118
gen.XM_059745,2131
gen.XM_059773,2141
gen.XM_059776,2062
gen.XM_059801,1939
gen.XM_059839,2430
gen.XM_059876,2282
gen.XM_059933,2531
gen.XM_059945,2838
gen.XM_059961,2859
gen.XM_059966,2871
gen.XM_059979,2644
gen.XM_059986,2813
gen.XM_059998,2673
gen.XM_060006,2647
gen.XM_060012,4115
gen.XM_060030,6146
gen.XM_060042,4281
gen.XM_060067,1499
gen.XM_060331,509
gen.XM_060517,531
gen.XM_060976,2885
gen.XM_061125,2931
gen.XM_061126,2930
gen.XM_062437,3775
gen.XM_063639,4234
gen.XM_064091,4597
gen.XM_065884,777
gen.XM_066291,5998
gen.XM_066900,6261
gen.XM_067264,1240
gen.XM_067325,5030
gen.XM_067715,1169
gen.XM_068164,1497
gen.XM_068395,1789
gen.XM_068853,1714
gen.XM_068919,2085
gen.XM_068963,2072
gen.XM_070188,2480
gen.XM_070203,2473
gen.XM_070873,2742
gen.XM_071178,2705
gen.XM_071580,1557
gen.XM_071605,2381
gen.XM_071623,1439
gen.XM_071801,4122
gen.XM_071873,4630
gen.XM_071937,2152
gen.XM_072173,5876
gen.XM_072430,2387
gen.XM_072526,2857
gen.XM_076414,1199
gen.XM_083842,3026
gen.XM_083852,3141
gen.XM_083864,3774
gen.XM_083866,3715
gen.XM_083868,3590
gen.XM_083892,3787
gen.XM_083939,4364
gen.XM_083966,4923
gen.XM_083983,4881
gen.XM_084007,5055
gen.XM_084014,5246
gen.XM_084023,5528
gen.XM_084026,5549
gen.XM_084055,580
gen.XM_084084,6090
gen.XM_084110,1340
gen.XM_084111,1243
gen.XM_084120,1315
gen.XM_084123,1263
gen.XM_084129,1231
gen.XM_084141,1041
gen.XM_084158,1465
gen.XM_084168,1547
gen.XM_084179,1591
gen.XM_084180,1781
gen.XM_084204,2079
gen.XM_084238,2453
gen.XM_084241,2337
gen.XM_084270,2851
gen.XM_084283,6229
gen.XM_084287,6203
gen.XM_084288,6153
gen.XM_084296,6227
gen.XM_084311,6350
gen.XM_084359,3073
gen.XM_084372,3016
gen.XM_084385,2944
gen.XM_084413,3028
gen.XM_084420,2910
gen.XM_084429,2911
gen.XM_084450,2942
gen.XM_084451,2953
gen.XM_084467,2994
gen.XM_084477,3010
gen.XM_084480,3012
gen.XM_084505,3080
gen.XM_084514,3180
gen.XM_084515,3183
gen.XM_084516,3182
gen.XM_084517,3184
gen.XM_084522,3424
gen.XM_084525,3428
gen.XM_084527,3169
gen.XM_084570,3357
gen.XM_084601,3353
gen.XM_084610,3350
gen.XM_084632,3072
gen.XM_084645,3731
gen.XM_084654,3388
gen.XM_084658,3382
gen.XM_084681,3195
gen.XM_084702,3287
gen.XM_084739,3124
gen.XM_084742,3122
gen.XM_084770,3515
gen.XM_084789,3599
gen.XM_084800,3783
gen.XM_084801,3672
gen.XM_084807,3531
gen.XM_084808,3818
gen.XM_084824,3630
gen.XM_084841,3540
gen.XM_084866,3557
gen.XM_084884,3583
gen.XM_084885,3582
gen.XM_084889,3814
gen.XM_084901,3488
gen.XM_084909,3702
gen.XM_084912,3705
gen.XM_084918,3500
gen.XM_084922,3495
gen.XM_084941,3788
gen.XM_084946,3800
gen.XM_084948,3804
gen.XM_084982,3870
gen.XM_084997,3933
gen.XM_084998,2142
gen.XM_085017,3893
gen.XM_085044,3916
gen.XM_085065,4044
gen.XM_085066,4033
gen.XM_085068,1480
gen.XM_085106,3987
gen.XM_085125,4031
gen.XM_085127,4014
gen.XM_085141,4019
gen.XM_085151,4050
gen.XM_085162,4054
gen.XM_085166,3955
gen.XM_085203,4130
gen.XM_085204,4132
gen.XM_085215,4282
gen.XM_085239,4254
gen.XM_085249,4236
gen.XM_085262,4314
gen.XM_085280,4289
gen.XM_085283,4211
gen.XM_085307,4160
gen.XM_085327,4622
gen.XM_085340,4448
gen.XM_085393,4480
gen.XM_085395,4482
gen.XM_085408,4637
gen.XM_085434,4524
gen.XM_085442,4513
gen.XM_085445,4425
gen.XM_085452,4435
gen.XM_085471,4558
gen.XM_085475,4561
gen.XM_085483,4616
gen.XM_085525,4323
gen.XM_085531,4977
gen.XM_085545,4741
gen.XM_085548,4735
gen.XM_085563,4991
gen.XM_085581,472
gen.XM_085589,4948
gen.XM_085613,4724
gen.XM_085627,4951
gen.XM_085636,4873
gen.XM_085672,4757
gen.XM_085687,4659
gen.XM_085691,4677
gen.XM_085716,4992
gen.XM_085722,4745
gen.XM_085735,5019
gen.XM_085743,4718
gen.XM_085775,5058
gen.XM_085779,5075
gen.XM_085788,5049
gen.XM_085789,5043
gen.XM_085790,5045
gen.XM_085791,5042
gen.XM_085856,5501
gen.XM_085862,5244
gen.XM_085874,5460
gen.XM_085875,5461
gen.XM_085876,5462
gen.XM_085909,5297
gen.XM_085916,5285
gen.XM_085917,5276
gen.XM_085927,5527
gen.XM_085928,5489
gen.XM_085934,5537
gen.XM_085935,5573
gen.XM_085950,5487
gen.XM_085971,5371
gen.XM_085972,5629
gen.XM_085981,4599
gen.XM_085986,5398
gen.XM_086004,5425
gen.XM_086074,5311
gen.XM_086101,5128
gen.XM_086102,5130
gen.XM_086116,5331
gen.XM_086132,304
gen.XM_086138,282
gen.XM_086142,557
gen.XM_086151,46
gen.XM_086164,277
gen.XM_086165,279
gen.XM_086166,281
gen.XM_086167,280
gen.XM_086178,4
gen.XM_086180,19
gen.XM_086204,38
gen.XM_086228,1356
gen.XM_086244,601
gen.XM_086245,602
gen.XM_086257,632
gen.XM_086271,383
gen.XM_086278,4434
gen.XM_086282,543
gen.XM_086296,331
gen.XM_086324,214
gen.XM_086328,542
gen.XM_086343,265
gen.XM_086357,85
gen.XM_086360,29
gen.XM_086375,97
gen.XM_086378,485
gen.XM_086381,479
gen.XM_086384,178
gen.XM_086389,243
gen.XM_086391,231
gen.XM_086397,323
gen.XM_086400,366
gen.XM_086428,2161
gen.XM_086431,589
gen.XM_086432,592
gen.XM_086444,136
gen.XM_086481,490
gen.XM_086484,494
gen.XM_086485,493
gen.XM_086494,538
gen.XM_086515,324
gen.XM_086518,317
gen.XM_086543,190
gen.XM_086552,432
gen.XM_086564,388
gen.XM_086567,430
gen.XM_086586,52
gen.XM_086587,54
gen.XM_086648,5819
gen.XM_086701,5687
gen.XM_086710,5670
gen.XM_086715,5695
gen.XM_086736,5717
gen.XM_086745,5712
gen.XM_086759,5877
gen.XM_086760,5878
gen.XM_086770,5914
gen.XM_086773,5928
gen.XM_086777,5930
gen.XM_086779,5064
gen.XM_086805,5963
gen.XM_086809,5953
gen.XM_086821,5985
gen.XM_086830,6043
gen.XM_086844,6074
gen.XM_086873,5964
gen.XM_086875,6093
gen.XM_086920,805
gen.XM_086923,849
gen.XM_086925,850
gen.XM_086944,933
gen.XM_086950,858
gen.XM_086961,926
gen.XM_086980,791
gen.XM_087028,942
gen.XM_087038,2803
gen.XM_087040,842
gen.XM_087041,2800
gen.XM_087045,932
gen.XM_087051,748
gen.XM_087061,912
gen.XM_087062,914
gen.XM_087068,775
gen.XM_087069,772
gen.XM_087118,891
gen.XM_087122,839
gen.XM_087151,683
gen.XM_087162,985
gen.XM_087166,993
gen.XM_087181,965
gen.XM_087193,726
gen.XM_087195,725
gen.XM_087206,669
gen.XM_087211,743
gen.XM_087218,1011
gen.XM_087240,901
gen.XM_087254,1302
gen.XM_087268,1203
gen.XM_087278,1358
gen.XM_087284,1075
gen.XM_087289,1323
gen.XM_087295,1322
gen.XM_087297,1360
gen.XM_087322,1312
gen.XM_087331,1211
gen.XM_087341,1267
gen.XM_087342,1265
gen.XM_087346,1115
gen.XM_087349,1106
gen.XM_087359,1343
gen.XM_087370,1101
gen.XM_087392,1333
gen.XM_087410,1347
gen.XM_087448,1184
gen.XM_087480,3000
gen.XM_087498,1463
gen.XM_087514,1483
gen.XM_087527,1455
gen.XM_087583,1418
gen.XM_087588,1120
gen.XM_087597,1549
gen.XM_087599,1551
gen.XM_087600,1553
gen.XM_087601,1550
gen.XM_087610,1597
gen.XM_087611,1595
gen.XM_087614,1564
gen.XM_087621,1711
gen.XM_087635,1660
gen.XM_087637,1662
gen.XM_087652,1713
gen.XM_087659,1537
gen.XM_087686,1543
gen.XM_087710,3247
gen.XM_087713,1559
gen.XM_087745,1656
gen.XM_087773,1816
gen.XM_087790,1631
gen.XM_087823,1858
gen.XM_087834,2123
gen.XM_087836,2124
gen.XM_087853,2090
gen.XM_087855,2089
gen.XM_087939,2000
gen.XM_087945,1990
gen.XM_087955,3857
gen.XM_087960,1883
gen.XM_087990,1936
gen.XM_087991,2154
gen.XM_088009,3106
gen.XM_088020,1621
gen.XM_088073,2386
gen.XM_088099,2416
gen.XM_088103,2418
gen.XM_088105,2409
gen.XM_088107,605
gen.XM_088119,2422
gen.XM_088122,2420
gen.XM_088135,2446
gen.XM_088180,2352
gen.XM_088239,2297
gen.XM_088264,2195
gen.XM_088294,2529
gen.XM_088316,2611
gen.XM_088321,2628
gen.XM_088323,2574
gen.XM_088325,2572
gen.XM_088336,2519
gen.XM_088338,2515
gen.XM_088370,2613
gen.XM_088399,2559
gen.XM_088401,2560
gen.XM_088422,2839
gen.XM_088426,2833
gen.XM_088459,2847
gen.XM_088461,2870
gen.XM_088472,1472
gen.XM_088550,2640
gen.XM_088552,2641
gen.XM_088553,2642
gen.XM_088563,2672
gen.XM_088569,2748
gen.XM_088571,2750
gen.XM_088587,4120
gen.XM_088588,4114
gen.XM_088589,4121
gen.XM_088592,6311
gen.XM_088619,6151
gen.XM_088622,6152
gen.XM_088630,6209
gen.XM_088637,2700
gen.XM_088638,768
gen.XM_088665,6158
gen.XM_088688,6220
gen.XM_088689,6218
gen.XM_088710,6253
gen.XM_088736,6265
gen.XM_088738,6267
gen.XM_088739,6268
gen.XM_088745,6289
gen.XM_088747,6128
gen.XM_088788,338
gen.XM_088863,286
gen.XM_088945,507
gen.XM_089030,622
gen.XM_089138,254
gen.XM_089514,3019
gen.XM_089551,3006
gen.XM_090218,3542
gen.XM_090413,3779
gen.XM_090458,3767
gen.XM_090833,638
gen.XM_090914,4082
gen.XM_090991,4191
gen.XM_091076,1091
gen.XM_091100,4263
gen.XM_091108,4124
gen.XM_091159,4157
gen.XM_091270,4483
gen.XM_091399,4590
gen.XM_091420,4544
gen.XM_091786,3426
gen.XM_091886,5595
gen.XM_091938,5221
gen.XM_091981,5586
gen.XM_091984,5396
gen.XM_092042,5108
gen.XM_092046,5341
gen.XM_092049,5380
gen.XM_092135,672
gen.XM_092158,918
gen.XM_092346,944
gen.XM_092489,867
gen.XM_092517,676
gen.XM_092545,970
gen.XM_092760,5696
gen.XM_092888,5986
gen.XM_092966,6113
gen.XM_093050,6212
gen.XM_093130,6226
gen.XM_093219,6299
gen.XM_093241,6228
gen.XM_093423,1308
gen.XM_093487,1255
gen.XM_093546,1201
gen.XM_093624,1083
gen.XM_094243,1797
gen.XM_094440,1561
gen.XM_094741,1862
gen.XM_094855,2060
gen.XM_095146,2432
gen.XM_095371,2475
gen.XM_095545,2514
gen.XM_095667,2554
gen.XM_096038,3699
gen.XM_096060,4241
gen.XM_096146,3539
gen.XM_096149,661
gen.XM_096155,5967
gen.XM_096156,5968
gen.XM_096169,1022
gen.XM_096172,787
gen.XM_096195,1190
gen.XM_096198,1117
gen.XM_096203,1464
gen.XM_096303,6256
gen.XM_096486,3315
gen.XM_096520,3165
gen.XM_096544,3119
gen.XM_096566,3680
gen.XM_096572,3819
gen.XM_096597,3739
gen.XM_096606,3608
gen.XM_096620,3578
gen.XM_096630,3486
gen.XM_096661,3441
gen.XM_096744,4034
gen.XM_096772,3966
gen.XM_096842,4245
gen.XM_096844,4286
gen.XM_097043,4984
gen.XM_097193,5001
gen.XM_097195,5000
gen.XM_097204,4754
gen.XM_097232,5048
gen.XM_097274,5510
gen.XM_097275,5521
gen.XM_097300,5222
gen.XM_097365,5440
gen.XM_097420,5134
gen.XM_097453,2068
gen.XM_097519,561
gen.XM_097565,249
gen.XM_097639,352
gen.XM_097649,198
gen.XM_097713,5800
gen.XM_097727,5773
gen.XM_097731,5795
gen.XM_097749,5644
gen.XM_097772,5731
gen.XM_097807,5929
gen.XM_097817,5925
gen.XM_097833,5950
gen.XM_097886,5971
gen.XM_097976,715
gen.XM_098004,729
gen.XM_098047,962
gen.XM_098048,960
gen.XM_098109,1345
gen.XM_098111,1245
gen.XM_098154,1232
gen.XM_098158,1103
gen.XM_098173,1227
gen.XM_098248,1384
gen.XM_098351,1609
gen.XM_098352,1611
gen.XM_098354,1610
gen.XM_098362,1634
gen.XM_098387,1778
gen.XM_098405,1534
gen.XM_098468,2108
gen.XM_098599,619
gen.XM_098654,2447
gen.XM_098669,2466
gen.XM_098747,2582
gen.XM_098761,2564
gen.XM_098913,2843
gen.XM_098943,2725
gen.XM_098995,6302
gen.XM_099467,363
gen.XM_102377,4432
gen.XM_103946,665
gen.XM_104983,6263
gen.XM_105236,1289
gen.XM_105658,1325
gen.XM_106246,1520
gen.XM_106739,1562
gen.XM_107825,2225
gen.XM_109162,3075
gen.XM_113223,3268
gen.XM_113224,3275
gen.XM_113226,3400
gen.XM_113229,3366
gen.XM_113230,3363
gen.XM_113238,3152
gen.XM_113266,4202
gen.XM_113268,4207
gen.XM_113291,4429
gen.XM_113293,4467
gen.XM_113299,4504
gen.XM_113303,5013
gen.XM_113310,4723
gen.XM_113315,4944
gen.XM_113324,4674
gen.XM_113325,4703
gen.XM_113328,4695
gen.XM_113330,5011
gen.XM_113334,4819
gen.XM_113343,5028
gen.XM_113348,5316
gen.XM_113352,5294
gen.XM_113360,386
gen.XM_113361,598
gen.XM_113369,361
gen.XM_113374,140
gen.XM_113379,473
gen.XM_113380,5749
gen.XM_113390,929
gen.XM_113395,1193
gen.XM_113397,1244
gen.XM_113405,1140
gen.XM_113408,1296
gen.XM_113409,1202
gen.XM_113410,1088
gen.XM_113417,1254
gen.XM_113422,1329
gen.XM_113425,1452
gen.XM_113452,1556
gen.XM_113454,1841
gen.XM_113463,1654
gen.XM_113467,1720
gen.XM_113468,1845
gen.XM_113476,1860
gen.XM_113531,2526
gen.XM_113532,2627
gen.XM_113540,2548
gen.XM_113557,2493
gen.XM_113564,2846
gen.XM_113585,6122
gen.XM_113615,2927
gen.XM_113702,3862
gen.XM_113712,3635
gen.XM_113719,3560
gen.XM_113726,3584
gen.XM_113730,3519
gen.XM_113737,3855
gen.XM_113739,3437
gen.XM_113752,3946
gen.XM_113759,4105
gen.XM_113823,4163
gen.XM_113836,4326
gen.XM_113840,4608
gen.XM_113843,4420
gen.XM_113845,4418
gen.XM_113853,4570
gen.XM_113855,4560
gen.XM_113874,4431
gen.XM_113876,4426
gen.XM_113882,4640
gen.XM_113892,4978
gen.XM_113901,4653
gen.XM_113919,4905
gen.XM_113929,4696
gen.XM_113931,4706
gen.XM_113938,4824
gen.XM_113943,5010
gen.XM_113945,4998
gen.XM_113951,4962
gen.XM_113988,5229
gen.XM_114004,5349
gen.XM_114018,5097
gen.XM_114024,5560
gen.XM_114025,5530
gen.XM_114027,5366
gen.XM_114030,560
gen.XM_114044,129
gen.XM_114055,384
gen.XM_114062,3
gen.XM_114097,376
gen.XM_114098,360
gen.XM_114109,525
gen.XM_114125,259
gen.XM_114137,634
gen.XM_114153,484
gen.XM_114154,5875
gen.XM_114163,5794
gen.XM_114165,5813
gen.XM_114174,5673
gen.XM_114178,5706
gen.XM_114185,5889
gen.XM_114209,6024
gen.XM_114215,816
gen.XM_114229,838
gen.XM_114247,824
gen.XM_114266,851
gen.XM_114267,856
gen.XM_114298,957
gen.XM_114301,1225
gen.XM_114309,1242
gen.XM_114323,1141
gen.XM_114328,1344
gen.XM_114356,1288
gen.XM_114364,1122
gen.XM_114368,1510
gen.XM_114401,1496
gen.XM_114424,1473
gen.XM_114426,1470
gen.XM_114434,1555
gen.XM_114435,1552
gen.XM_114437,1567
gen.XM_114439,1586
gen.XM_114440,1587
gen.XM_114442,1584
gen.XM_114453,1819
gen.XM_114457,1817
gen.XM_114469,1623
gen.XM_114482,1683
gen.XM_114492,2106
gen.XM_114497,2058
gen.XM_114555,2429
gen.XM_114578,2444
gen.XM_114602,2404
gen.XM_114613,2625
gen.XM_114617,2517
gen.XM_114618,2523
gen.XM_114640,2556
gen.XM_114646,2756
gen.XM_114649,2873
gen.XM_114655,2854
gen.XM_114661,2677
gen.XM_114662,2688
gen.XM_114669,2845
gen.XM_114677,2802
gen.XM_114678,2801
gen.XM_114679,2799
gen.XM_114686,2699
gen.XM_114692,6354
gen.XM_114708,6291
gen.XM_114720,6130
gen.XM_114724,6119
gen.XM_114798,233
gen.XM_114862,3104
gen.XM_114894,2977
gen.XM_114981,3139
gen.XM_115031,3286
gen.XM_115062,3364
gen.XM_115063,3365
gen.XM_115081,3177
gen.XM_115117,3570
gen.XM_115140,3634
gen.XM_115197,3809
gen.XM_115215,3948
gen.XM_115352,4333
gen.XM_115480,4910
gen.XM_115603,5466
gen.XM_115615,5395
gen.XM_115672,869
gen.XM_115706,1039
gen.XM_115722,1040
gen.XM_115825,1002
gen.XM_115846,5691
gen.XM_115874,6281
gen.XM_115886,6131
gen.XM_115890,6136
gen.XM_115923,6259
gen.XM_115924,6121
gen.XM_116034,1338
gen.XM_116058,1295
gen.XM_116071,1204
gen.XM_116072,1205
gen.XM_116204,1532
gen.XM_116205,1533
gen.XM_116247,1484
gen.XM_116285,1408
gen.XM_116307,1691
gen.XM_116340,1807
gen.XM_116365,1856
gen.XM_116427,1648
gen.XM_116439,1593
gen.XM_116447,1606
gen.XM_116465,1716
gen.XM_116511,1857
gen.XM_116514,1861
gen.XM_116524,2140
gen.XM_116806,2789
gen.XM_116818,2738
gen.XM_116853,1139
gen.XM_116856,1810
gen.XM_116863,2975
gen.XM_116913,3845
gen.XM_116926,3451
gen.XM_117061,4913
gen.XM_117066,4768
gen.XM_117096,5084
gen.XM_117118,5379
gen.XM_117122,5183
gen.XM_117128,5605
gen.XM_117159,2
gen.XM_117181,534
gen.XM_117184,163
gen.XM_117185,582
gen.XM_117196,641
gen.XM_117209,5688
gen.XM_117264,736
gen.XM_117311,1337
gen.XM_117351,1412
gen.XM_117387,1622
gen.XM_117398,1641
gen.XM_117444,2471
gen.XM_117449,2160
gen.XM_117452,2472
gen.XM_117481,2406
gen.XM_117487,2622
gen.XM_117519,2874
gen.XM_117539,6352
gen.XM_117555,6349
gen.XM_117692,28
gen.XM_118637,4251
gen.XM_165390,3427
gen.XM_165410,4583
gen.XM_165411,4413
gen.XM_165418,4713
gen.XM_165421,4701
gen.XM_165422,4704
gen.XM_165432,5541
gen.XM_165438,144
gen.XM_165439,620
gen.XM_165442,59
gen.XM_165443,477
gen.XM_165448,723
gen.XM_165451,1268
gen.XM_165465,1531
gen.XM_165470,1528
gen.XM_165473,1482
gen.XM_165483,1818
gen.XM_165484,1820
gen.XM_165488,1615
gen.XM_165499,2057
gen.XM_165514,2579
gen.XM_165530,6355
gen.XM_165533,6235
gen.XM_165551,2913
gen.XM_165555,2889
gen.XM_165557,2897
gen.XM_165560,2925
gen.XM_165563,2926
gen.XM_165567,2921
gen.XM_165571,3407
gen.XM_165584,3414
gen.XM_165586,3413
gen.XM_165592,3401
gen.XM_165598,3303
gen.XM_165600,3310
gen.XM_165610,3222
gen.XM_165611,3217
gen.XM_165612,3223
gen.XM_165616,3325
gen.XM_165627,3335
gen.XM_165628,3341
gen.XM_165631,3328
gen.XM_165636,3903
gen.XM_165639,3917
gen.XM_165645,4534
gen.XM_165647,4528
gen.XM_165648,4537
gen.XM_165649,4527
gen.XM_165656,4484
gen.XM_165657,4493
gen.XM_165658,4489
gen.XM_165669,2091
gen.XM_165692,2159
gen.XM_165698,1949
gen.XM_165717,1954
gen.XM_165728,2036
gen.XM_165738,1999
gen.XM_165740,1865
gen.XM_165743,1937
gen.XM_165747,1948
gen.XM_165749,2037
gen.XM_165758,2013
gen.XM_165764,2011
gen.XM_165765,1988
gen.XM_165770,1951
gen.XM_165771,1983
gen.XM_165772,1876
gen.XM_165777,2044
gen.XM_165794,1921
gen.XM_165799,2006
gen.XM_165801,1956
gen.XM_165809,2016
gen.XM_165836,2350
gen.XM_165839,2346
gen.XM_165841,2197
gen.XM_165860,2167
gen.XM_165867,2249
gen.XM_165870,2245
gen.XM_165872,2253
gen.XM_165876,2258
gen.XM_165877,2240
gen.XM_165882,2248
gen.XM_165888,2934
gen.XM_165890,2929
gen.XM_165891,2941
gen.XM_165903,3633
gen.XM_165905,3579
gen.XM_165906,3532
gen.XM_165910,3465
gen.XM_165921,4127
gen.XM_165923,4325
gen.XM_165954,5026
gen.XM_165960,5347
gen.XM_165963,5367
gen.XM_165975,327
gen.XM_165976,373
gen.XM_165977,264
gen.XM_165978,532
gen.XM_165981,290
gen.XM_165983,275
gen.XM_165984,175
gen.XM_165994,927
gen.XM_165998,893
gen.XM_166007,910
gen.XM_166008,900
gen.XM_166011,1121
gen.XM_166014,1275
gen.XM_166015,1192
gen.XM_166017,1350
gen.XM_166026,1669
gen.XM_166027,1663
gen.XM_166028,1842
gen.XM_166029,1802
gen.XM_166037,1612
gen.XM_166042,2054
gen.XM_166049,2147
gen.XM_166063,2540
gen.XM_166064,2558
gen.XM_166078,6142
gen.XM_166081,6255
gen.XM_166093,2984
gen.XM_166125,2966
gen.XM_166157,2922
gen.XM_166174,3409
gen.XM_166177,3406
gen.XM_166181,3403
gen.XM_166196,3308
gen.XM_166232,3227
gen.XM_166234,3224
gen.XM_166235,3293
gen.XM_166236,3294
gen.XM_166239,3349
gen.XM_166253,3336
gen.XM_166266,3904
gen.XM_166273,3886
gen.XM_166277,4532
gen.XM_166282,4491
gen.XM_166285,4490
gen.XM_166288,5071
gen.XM_166303,2092
gen.XM_166310,2101
gen.XM_166327,2157
gen.XM_166333,1932
gen.XM_166336,2021
gen.XM_166340,1882
gen.XM_166349,1872
gen.XM_166353,2002
gen.XM_166357,2049
gen.XM_166360,1938
gen.XM_166361,2009
gen.XM_166362,1884
gen.XM_166363,1940
gen.XM_166376,2004
gen.XM_166381,1992
gen.XM_166392,2019
gen.XM_166401,1995
gen.XM_166402,1896
gen.XM_166406,2015
gen.XM_166412,1910
gen.XM_166417,1914
gen.XM_166419,1920
gen.XM_166425,1888
gen.XM_166446,2042
gen.XM_166457,1878
gen.XM_166459,1931
gen.XM_166469,1879
gen.XM_166480,1955
gen.XM_166482,2351
gen.XM_166485,2353
gen.XM_166494,2224
gen.XM_166504,2222
gen.XM_166505,2202
gen.XM_166506,2200
gen.XM_166509,2219
gen.XM_166512,2205
gen.XM_166513,2220
gen.XM_166514,2203
gen.XM_166515,2204
gen.XM_166521,2198
gen.XM_166523,2170
gen.XM_166531,2190
gen.XM_166540,2191
gen.XM_166541,2168
gen.XM_166594,2230
gen.XM_166599,20
gen.XM_166605,3506
gen.XM_166629,2988
gen.XM_166665,2918
gen.XM_166717,2906
gen.XM_166743,3418
gen.XM_167008,5080
gen.XM_167016,2087
gen.XM_167027,2094
gen.XM_167037,2096
gen.XM_167046,2150
gen.XM_167128,2023
gen.XM_167161,2025
gen.XM_167169,1868
gen.XM_167179,2031
gen.XM_167196,2041
gen.XM_167225,2047
gen.XM_167339,2264
gen.XM_167363,5065
gen.XM_167366,1209
gen.XM_167374,2898
gen.XM_167395,2963
gen.XM_167411,2901
gen.XM_167414,2904
gen.XM_167433,3324
gen.XM_167437,3192
gen.XM_167439,3876
gen.XM_167453,4538
gen.XM_167456,4541
gen.XM_167476,2321
gen.XM_167477,2325
gen.XM_167483,2328
gen.XM_167484,2329
gen.XM_167494,2273
gen.XM_167498,2301
gen.XM_167500,2299
gen.XM_167502,2312
gen.XM_167504,2300
gen.XM_167518,3754
gen.XM_167530,5529
gen.XM_167538,5945
gen.XM_167558,2645
gen.XM_167626,2887
gen.XM_167716,3244
gen.XM_167726,3248
gen.XM_167747,3234
gen.XM_167748,3228
gen.XM_167780,3417
gen.XM_167804,3291
gen.XM_167853,3318
gen.XM_167892,3883
gen.XM_167906,3877
gen.XM_167911,3868
gen.XM_167918,3869
gen.XM_168054,2103
gen.XM_168070,1928
gen.XM_168104,1994
gen.XM_168123,1877
gen.XM_168181,2322
gen.XM_168251,2323
gen.XM_168354,2271
gen.XM_168378,2269
gen.XM_168435,2316
gen.XM_168450,2315
gen.XM_168454,2302
gen.XM_168461,2311
gen.XM_168464,2317
gen.XM_168470,2310
gen.XM_168548,2375
gen.XM_168572,2380
gen.XM_168586,2360
gen.XM_169414,3880
gen.XM_169540,5078
gen.XM_170195,2267
gen.XM_170427,2318

### DETAILED DESCRIPTION OF THE PREFERRED EMBODIMENTS

I. Definitions

The terms “TAT polypeptide” and “TAT” as used herein and when immediately followed by a numerical designation,refer to various polypeptides, wherein the complete designation (i.e.,TAT/number) refers to specific polypeptide sequences as described herein. The terms “TAT/number polypeptide” and “TAT/number” wherein the term “number” is provided as an actual numerical designation as used herein encompass native sequence polypeptides, polypeptide variants and fragments of native sequence polypeptides and polypeptide variants (which are further defined herein). The TAT polypeptides described herein may be isolated from a variety of sources, such as from human tissue types or from another source, or prepared by recombinant or synthetic methods. The term “TAT polypeptide” refers to each individual TAT/number polypeptide disclosed herein. All disclosures in this specification which refer to the “TAT polypeptide” refer to each of the polypeptides individually as well as jointly. For example, descriptions of the preparation of, purification of, derivation of, formation of antibodies to or against, formation of TAT binding oligopeptides to or against, formation of TAT binding organic molecules to or against, administration of, compositions containing, treatment of a disease with, etc., pertain to each polypeptide of the invention individually. The term “TAT polypeptide” also includes variants of the TAT/number polypeptides disclosed herein.

A “native sequence TAT polypeptide” comprises a polypeptide having the same amino acid sequence as the corresponding TAT polypeptide derived from nature. Such native sequence TAT polypeptides can be isolated from nature or can be produced by recombinant or synthetic means. The term “native sequence TAT polypeptide” specifically encompasses naturally-occurring truncated or secreted forms of the specific TAT polypeptide (e.g., an extracellular domain sequence), naturally-occurring variant forms (e.g., alternatively spliced forms) and naturally-occurring allelic variants of the polypeptide. In certain embodiments of the invention, the native sequence TAT polypeptides disclosed herein are mature or full-length native sequence polypeptides comprising the full-length amino acids sequences shown in the accompanying figures. Start and stop codons (if indicated) are shown in bold font and underlined in the figures. Nucleic acid residues indicated as “N” in the accompanying figures are any nucleic acid residue. However, while the TAT polypeptides disclosed in the accompanying figures are shown to begin with methionine residues designated herein as amino acid position 1 in the figures, it is conceivable and possible that other methionine residues located either upstream or downstream from the amino acid position 1 in the figures may be employed as the starting amino acid residue for the TAT polypeptides.

The TAT polypeptide “extracellular domain” or “ECD” refers to a form of the TAT polypeptide which is essentially free of the transmembrane and cytoplasmic domains. Ordinarily, a TAT polypeptide ECD will have less than 1% of such transmembrane and/or cytoplasmic domains and preferably, will have less than 0.5% of such domains. It will be understood that any transmembrane domains identified for the TAT polypeptides of the present invention are identified pursuant to criteria routinely employed in the art for identifying that type of hydrophobic domain. The exact boundaries of a transmembrane domain may vary but most likely by no more than about 5 amino acids at either end of the domain as initially identified herein. Optionally, therefore, an extracellular domain of a TAT polypeptide may contain from about 5 or fewer amino acids on either side of the transmembrane domain/extracellular domain boundary as identified in the Examples or specification and such polypeptides, with or without the associated signal peptide, and nucleic acid encoding them, are contemplated by the present invention.

The approximate location of the “signal peptides” of the various TAT polypeptides disclosed herein may be shown in the present specification and/or the accompanying figures. It is noted, however, that the C-terminal boundary of a signal peptide may vary, but most likely by no more than about 5 amino acids on either side of the signal peptide C-terminal boundary as initially identified herein, wherein the C-terminal boundary of the signal peptide may be identified pursuant to criteria routinely employed in the art for identifying that type of amino acid sequence element (e.g., Nielsen et al., Prot. Eng. 10:1-6 (1997) and von Heinje et al., Nucl. Acids. Res. 14:4683-4690 (1986)). Moreover, it is also recognized that, in some cases, cleavage of a signal sequence from a secreted polypeptide is not entirely uniform, resulting in more than one secreted species. These mature polypeptides, where the signal peptide is cleaved within no more than about 5 amino acids on either side of the C-terminal boundary of the signal peptide as identified herein, and the polynucleotides encoding them, are contemplated by the present invention.

“TAT polypeptide variant” means a TAT polypeptide, preferably an active TAT polypeptide, as defined herein having at least about 80% amino acid sequence identity with a full-length native sequence TAT polypeptide sequence as disclosed herein, a TAT polypeptide sequence lacking the signal peptide as disclosed herein, an extracellular domain of a TAT polypeptide, with or without the signal peptide, as disclosed herein or any other fragment of a full-length TAT polypeptide sequence as disclosed herein (such as those encoded by a nucleic acid that represents only a portion of the complete coding sequence for a full-length TAT polypeptide). Such TAT polypeptide variants include, for instance, TAT polypeptides wherein one or more amino acid residues are added, or deleted, at the N- or C-terminus of the full-length native amino acid sequence. Ordinarily, a TAT polypeptide variant will have at least about 80% amino acid sequence identity, alternatively at least about 81%, 82%, 83%, 84%, 85%, 86%, 87%, 88%, 89%, 90%, 91%, 92%, 93%, 94%, 95%, 96%, 97%, 98%, or 99% amino acid sequence identity, to a full-length native sequence TAT polypeptide sequence as disclosed herein, a TAT polypeptide sequence lacking the signal peptide as disclosed herein, an extracellular domain of a TAT polypeptide, with or without the signal peptide, as disclosed herein or any other specifically defined fragment of a full-length TAT polypeptide sequence as disclosed herein. Ordinarily, TAT variant polypeptides are at least about 10 amino acids in length, alternatively at least about 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600 amino acids in length, or more. Optionally, TAT variant polypeptides will have no more than one conservative amino acid substitution as compared to the native TAT polypeptide sequence, alternatively no more than 2, 3, 4, 5, 6, 7, 8, 9, or 10 conservative amino acid substitution as compared to the native TAT polypeptide sequence.

“Percent (%) amino acid sequence identity” with respect to the TAT polypeptide sequences identified herein is defined as the percentage of amino acid residues in a candidate sequence that are identical with the amino acid residues in the specific TAT polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for measuring alignment, including any algorithms needed to achieve maximal alignment over the full length of the sequences being compared. For purposes herein, however, % amino acid sequence identity values are generated using the sequence comparison computer program ALIGN-2, wherein the complete source code for the ALIGN-2 program is provided in Table 1 below. The ALIGN-2 sequence comparison computer program was authored by Genentech, Inc. and the source code shown in Table 1 below has been filed with user documentation in the U.S. Copyright Office, Washington D.C., 20559, where it is registered under U.S. Copyright Registration No. TXU510087. The ALIGN-2 program is publicly available through Genentech, Inc., South San Francisco, Calif. or may be compiled from the source code provided in Table 1 below. The ALIGN-2 program should be compiled for use on a UNIX operating system, preferably digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:
100 times the fraction X/Y
where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. As examples of % amino acid sequence identity calculations using this method, Tables 2 and 3 demonstrate how to calculate the % amino acid sequence identity of the amino acid sequence designated “Comparison Protein” to the amino acid sequence designated “TAT”, wherein “TAT” represents the amino acid sequence of a hypothetical TAT polypeptide of interest, “Comparison Protein” represents the amino acid sequence of a polypeptide against which the “TAT” polypeptide of interest is being compared, and “X, “Y” and “Z” each represent different hypothetical amino acid residues. Unless specifically stated otherwise, all % amino acid sequence identity values used herein are obtained as described in the immediately preceding paragraph using the ALIGN-2 computer program.

“TAT variant polynucleotide” or “TAT variant nucleic acid sequence” means a nucleic acid molecule which encodes a TAT polypeptide, preferably an active TAT polypeptide, as defined herein and which has at least about 80% nucleic acid sequence identity with a nucleotide acid sequence encoding a full-length native sequence TAT polypeptide sequence as disclosed herein, a full-length native sequence TAT polypeptide sequence lacking the signal peptide as disclosed herein, an extracellular domain of a TAT polypeptide, with or without the signal peptide, as disclosed herein or any other fragment, of a full-length TAT polypeptide sequence as disclosed herein (such as those encoded by a nucleic acid that represents only a portion of the complete coding sequence for a full-length TAT polypeptide). Ordinarily, a TAT variant polynucleotide will have at least about 80% nucleic acid sequence identity, alternatively at least about 81%, 82%, 83%, 84%, 85%, 86%, 87%, 88%, 89%, 90%, 91%, 92%, 93%, 94%, 95%, 96%, 97%, 98%, or 99% nucleic acid sequence identity with a nucleic acid sequence encoding a full-length native sequence TAT polypeptide sequence as disclosed herein, a full-length native sequence TAT polypeptide sequence lacking the signal peptide as disclosed herein, an extracellular domain of a TAT polypeptide, with or without the signal sequence, as disclosed herein or any other fragment of a full-length TAT polypeptide sequence as disclosed herein. Variants do not encompass the native nucleotide sequence.

Ordinarily, TAT variant polynucleotides are at least about 5 nucleotides in length, alternatively at least about 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 nucleotides in length, wherein in this context the term “about” means the referenced nucleotide sequence length plus or minus 10% of that referenced length.

“Percent (%) nucleic acid sequence identity” with respect to TAT-encoding nucleic acid sequences identified herein is defined as the percentage of nucleotides in a candidate sequence that are identical with the nucleotides in the TAT nucleic acid sequence of interest, after aligning the sequences and introducing gaps, if necessary, to achieve the maximum percent sequence identity. Alignment for purposes of determining percent nucleic acid sequence identity can be achieved in various ways that are within the skill in the art, for instance, using publicly available computer software such as BLAST, BLAST-2, ALIGN or Megalign (DNASTAR) software. For purposes herein, however, % nucleic acid sequence identity values are generated using the sequence comparison computer program ALIGN-2, wherein the complete source code for the ALIGN-2 program is provided in Table 1 below. The ALIGN-2 sequence comparison computer program was authored by Genentech, Inc. and the source code shown in Table 1 below has been filed with user documentation in the U.S. Copyright Office, Washington D.C., 20559, where it is registered under U.S. Copyright Registration No. TXU510087. The ALIGN-2 program is publicly available through Genentech, Inc., South San Francisco, Calif. or may be compiled from the source code provided in Table 1 below. The ALIGN-2 program should be compiled for use on a UNIX operating system, preferably digital UNIX V4.0D. All sequence comparison parameters are set by the ALIGN-2 program and do not vary.

In situations where ALIGN-2 is employed for nucleic acid sequence comparisons, the % nucleic acid sequence identity of a given nucleic acid sequence C to, with, or against a given nucleic acid sequence D (which can alternatively be phrased as a given nucleic acid sequence C that has or comprises a certain % nucleic acid sequence identity to, with, or against a given nucleic acid sequence D) is calculated as follows:
100 times the fraction W/Z
where W is the number of nucleotides scored as identical matches by the sequence alignment program ALIGN-2 in that program's alignment of C and D, and where Z is the total number of nucleotides in D. It will be appreciated that where the length of nucleic acid sequence C is not equal to the length of nucleic acid sequence D, the % nucleic acid sequence identity of C to D will not equal the % nucleic acid sequence identity of D to C. As examples of % nucleic acid sequence identity calculations, Tables 4 and 5, demonstrate how to calculate the % nucleic acid sequence identity of the nucleic acid sequence designated “Comparison DNA” to the nucleic acid sequence designated “TAT-DNA”, wherein “TAT-DNA” represents a hypothetical TAT-encoding nucleic acid sequence of interest, “Comparison DNA” represents the nucleotide sequence of a nucleic acid molecule against which the “TAT-DNA” nucleic acid molecule of interest is being compared, and “N”, “L” and “V” each represent different hypothetical nucleotides. Unless specifically stated otherwise, all % nucleic acid sequence identity values used herein are obtained as described in the immediately preceding paragraph using the ALIGN-2 computer program.

In other embodiments, TAT variant polynucleotides are nucleic acid molecules that encode a TAT polypeptide and which are capable of hybridizing, preferably under stringent hybridization and wash conditions, to nucleotide sequences encoding a full-length TAT polypeptide as disclosed herein. TAT variant polypeptides may be those that are encoded by a TAT variant polynucleotide.

The term “full-length coding region” when used in reference to a nucleic acid encoding a TAT polypeptide refers to the sequence of nucleotides which encode the full-length TAT polypeptide of the invention (which is often shown between start and stop codons, inclusive thereof, in the accompanying figures). The term “full-length coding region” when used in reference to an ATCC deposited nucleic acid refers to the TAT polypeptide-encoding portion of the cDNA that is inserted into the vector deposited with the ATCC (which is often shown between start and stop codons, inclusive thereof, in the accompanying figures).

“Isolated,” when used to describe the various TAT polypeptides disclosed herein, means polypeptide that has been identified and separated and/or recovered from a component of its natural environment. Contaminant components of its natural environment are materials that would typically interfere with diagnostic or therapeutic uses for the polypeptide, and may include enzymes, hormones, and other proteinaceous or non-proteinaceous solutes. In preferred embodiments, the polypeptide will be purified (1) to a degree sufficient to obtain at least 15 residues of N-terminal or internal amino acid sequence by use of a spinning cup sequenator, or (2) to homogeneity by SDS-PAGE under non-reducing or reducing conditions using Coomassie blue or, preferably, silver stain. Isolated polypeptide includes polypeptide in situ within recombinant cells, since at least one component of the TAT polypeptide natural environment will not be present. Ordinarily, however, isolated polypeptide will be prepared by at least one purification step.

An “isolated” TAT polypeptide-encoding nucleic acid or other polypeptide-encoding nucleic acid is a nucleic acid molecule that is identified and separated from at least one contaminant nucleic acid molecule with which it is ordinarily associated in the natural source of the polypeptide-encoding nucleic acid. An isolated polypeptide-encoding nucleic acid molecule is other than in the form or setting in which it is found in nature. Isolated polypeptide-encoding nucleic acid molecules therefore are distinguished from the specific polypeptide-encoding nucleic acid molecule as it exists in natural cells. However, an isolated polypeptide-encoding nucleic acid molecule includes polypeptide-encoding nucleic acid molecules contained in cells that ordinarily express the polypeptide where, for example, the nucleic acid molecule is in a chromosomal location different from that of natural cells.

The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

“Stringency” of hybridization reactions is readily determinable by one of ordinary skill in the art, and generally is an empirical calculation dependent upon probe length, washing temperature, and salt concentration. In general, longer probes require higher temperatures for proper annealing, while shorter probes need lower temperatures. Hybridization generally depends on the ability of denatured DNA to reanneal when complementary strands are present in an environment below their melting temperature. The higher the degree of desired homology between the probe and hybridizable sequence, the higher the relative temperature which can be used. As a result, it follows that higher relative temperatures would tend to make the reaction conditions more stringent, while lower temperatures less so. For additional details and explanation of stringency of hybridization reactions, see Ausubel et al., Current Protocols in Molecular Biology, Wiley Interscience Publishers, (1995).

“Stringent conditions” or “high stringency conditions”, as defined herein, may be identified by those that: (1) employ low ionic strength and high temperature for washing, for example 0.015 M sodium chloride/0.0015 M sodium citrate/0.1% sodium dodecyl sulfate at 50° C.; (2) employ during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or (3) overnight hybridization in a solution that employs 50% formamide, 5×SSC (0.75 M NaCl, 0.075 M sodium citrate), 50 mM sodium phosphate (pH 6.8), 0.1% sodium pyrophosphate, 5× Denhardt's solution, sonicated salmon sperm DNA (50 μg/ml), 0.1% SDS, and 10% dextran sulfate at 42° C., with a 10 minute wash at 42° C. in 0.2×SSC (sodium chloride/sodium citrate) followed by a 10 minute high-stringency wash consisting of 0.1×SSC containing EDTA at 55° C.

“Moderately stringent conditions” may be identified as described by Sambrook et al., Molecular Cloning: A Laboratory Manual, New York: Cold Spring Harbor Press, 1989, and include the use of washing solution and hybridization conditions (e.g., temperature, ionic strength and % SDS) less stringent that those described above. An example of moderately stringent conditions is overnight incubation at 37° C. in a solution comprising: 20% formamide, 5×SSC (150 mM NaCl, 15 mM trisodium citrate), 50 mM sodium phosphate (pH 7.6), 5× Denhardt's solution, 10% dextran sulfate, and 20 mg/ml denatured sheared salmon sperm DNA, followed by washing the filters in 1×SSC at about 37-50° C. The skilled artisan will recognize how to adjust the temperature, ionic strength, etc. as necessary to accommodate factors such as probe length and the like.

The term “epitope tagged” when used herein refers to a chimeric polypeptide comprising a TAT polypeptide or anti-TAT antibody fused to a “tag polypeptide”. The tag polypeptide has enough residues to provide an epitope against which an antibody can be made, yet is short enough such that it does not interfere with activity of the polypeptide to which it is fused. The tag polypeptide preferably also is fairly unique so that the antibody does not substantially cross-react with other epitopes. Suitable tag polypeptides generally have at least six amino acid residues and usually between about 8 and 50 amino acid residues (preferably, between about 10 and 20 amino acid residues).

“Active” or “activity” for the purposes herein refers to form(s) of a TAT polypeptide which retain a biological and/or an immunological activity of native or naturally-occurring TAT, wherein “biological” activity refers to a biological function (either inhibitory or stimulatory) caused by a native or naturally-occurring TAT other than the ability to induce the production of an antibody against an antigenic epitope possessed by a native or naturally-occurring TAT and an “immunological” activity refers to the ability to induce the production of an antibody against an antigenic epitope possessed by a native or naturally-occurring TAT.

The term “antagonist” is used in the broadest sense, and includes any molecule that partially or fully blocks, inhibits, or neutralizes a biological activity of a native TAT polypeptide disclosed herein. In a similar manner, the term “agonist” is used in the broadest sense and includes any molecule that mimics a biological activity of a native TAT polypeptide disclosed herein. Suitable agonist or antagonist molecules specifically include agonist or antagonist antibodies or antibody fragments, fragments or amino acid sequence variants of native TAT polypeptides, peptides, antisense oligonucleotides, small organic molecules, etc. Methods for identifying agonists or antagonists of a TAT polypeptide may comprise contacting a TAT polypeptide with a candidate agonist or antagonist molecule and measuring a detectable change in one or more biological activities normally associated with the TAT polypeptide.

“Treating” or “treatment” or “alleviation” refers to both therapeutic treatment and prophylactic or preventative measures, wherein the object is to prevent or slow down (lessen) the targeted pathologic condition or disorder. Those in need of treatment include those already with the disorder as well as those prone to have the disorder or those in whom the disorder is to be prevented. A subject or mammal is successfully “treated” for a TAT polypeptide-expressing cancer if, after receiving a therapeutic amount of an anti-TAT antibody, TAT binding oligopeptide or TAT binding organic molecule according to the methods of the present invention, the patient shows observable and/or measurable reduction in or absence of one or more of the following: reduction in the number of cancer cells or absence of the cancer cells; reduction in the tumor size; inhibition (i.e., slow to some extent and preferably stop) of cancer cell infiltration into peripheral organs including the spread of cancer into soft tissue and bone; inhibition (i.e., slow to some extent and preferably stop) of tumor metastasis; inhibition, to some extent, of tumor growth; and/or relief to some extent, one or more of the symptoms associated with the specific cancer; reduced morbidity and mortality, and improvement in quality of life issues. To the extent the anti-TAT antibody or TAT binding oligopeptide may prevent growth and/or kill existing cancer cells, it may be cytostatic and/or cytotoxic. Reduction of these signs or symptoms may also be felt by the patient.

The above parameters for assessing successful treatment and improvement in the disease are readily measurable by routine procedures familiar to a physician. For cancer therapy, efficacy can be measured, for example, by assessing the time to disease progression (TTP) and/or determining the response rate (RR). Metastasis can be determined by staging tests and by bone scan and tests for calcium level and other enzymes to determine spread to the bone. CT scans can also be done to look for spread to the pelvis and lymph nodes in the area. Chest X-rays and measurement of liver enzyme levels by known methods are used to look for metastasis to the lungs and liver, respectively. Other routine methods for monitoring the disease include transrectal ultrasonography (TRUS) and transrectal needle biopsy (TRNB).

For bladder cancer, which is a more localized cancer, methods to determine progress of disease include urinary cytologic evaluation by cystoscopy, monitoring for presence of blood in the urine, visualization of the urothelial tract by sonography or an intravenous pyelogram, computed tomography (CT) and magnetic resonance imaging (MRI). The presence of distant metastases can be assessed by CT of the abdomen, chest x-rays, or radionuclide imaging of the skeleton.

“Chronic” administration refers to administration of the agent(s) in a continuous mode as opposed to an acute mode, so as to maintain the initial therapeutic effect (activity) for an extended period of time. “Intermittent” administration is treatment that is not consecutively done without interruption, but rather is cyclic in nature.

“Mammal” for purposes of the treatment of, alleviating the symptoms of or diagnosis of a cancer refers to any animal classified as a mammal, including humans, domestic and farm animals, and zoo, sports, or pet animals, such as dogs, cats, cattle, horses, sheep, pigs, goats, rabbits, etc. Preferably, the mammal is human.

Administration “in combination with” one or more further therapeutic agents includes simultaneous (concurrent) and consecutive administration in any order.

“Carriers” as used herein include pharmaceutically acceptable carriers, excipients, or stabilizers which are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN®, polyethylene glycol (PEG), and PLURONICS®.

By “solid phase” or “solid support” is meant a non-aqueous matrix to which an antibody, TAT binding oligopeptide or TAT binding organic molecule of the present invention can adhere or attach. Examples of solid phases encompassed herein include those formed partially or entirely of glass (e.g., controlled pore glass), polysaccharides (e.g., agarose), polyacrylamides, polystyrene, polyvinyl alcohol and silicones. In certain embodiments, depending on the context, the solid phase can comprise the well of an assay plate; in others it is a purification column (e.g., an affinity chromatography column). This term also includes a discontinuous solid phase of discrete particles, such as those described in U.S. Pat. No. 4,275,149.

A “liposome” is a small vesicle composed of various types of lipids, phospholipids and/or surfactant which is useful for delivery of a drug (such as a TAT polypeptide, an antibody thereto or a TAT binding oligopeptide) to a mammal. The components of the liposome are commonly arranged in a bilayer formation, similar to the lipid arrangement of biological membranes.

A “small” molecule or “small” organic molecule is defined herein to have a molecular weight below about 500 Daltons.

An “effective amount” of a polypeptide, antibody, TAT binding oligopeptide, TAT binding organic molecule or an agonist or antagonist thereof as disclosed herein is an amount sufficient to carry out a specifically stated purpose. An “effective amount” may be determined empirically and in a routine manner, in relation to the stated purpose.

The term “therapeutically effective amount” refers to an amount of an antibody, polypeptide, TAT binding oligopeptide, TAT binding organic molecule or other drug effective to “treat” a disease or disorder in a subject or mammal. In the case of cancer, the therapeutically effective amount of the drug may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. See the definition herein of “treating”. To the extent the drug may prevent growth and/or kill existing cancer cells, it may be cytostatic and/or cytotoxic.

A “growth inhibitory amount” of an anti-TAT antibody, TAT polypeptide, TAT binding oligopeptide or TAT binding organic molecule is an amount capable of inhibiting the growth of a cell, especially tumor, e.g., cancer cell, either in vitro or in vivo. A “growth inhibitory amount” of an anti-TAT antibody, TAT polypeptide, TAT binding oligopeptide or TAT binding organic molecule for purposes of inhibiting neoplastic cell growth may be determined empirically and in a routine manner.

A “cytotoxic amount” of an anti-TAT antibody, TAT polypeptide, TAT binding oligopeptide or TAT binding organic molecule is an amount capable of causing the destruction of a cell, especially tumor, e.g., cancer cell, either in vitro or in vivo. A “cytotoxic amount” of an anti-TAT antibody, TAT polypeptide, TAT binding oligopeptide or TAT binding organic molecule for purposes of inhibiting neoplastic cell growth may be determined empirically and in a routine manner.

The term “antibody” is used in the broadest sense and specifically covers, for example, single anti-TAT monoclonal antibodies (including agonist, antagonist, and neutralizing antibodies), anti-TAT antibody compositions with polyepitopic specificity, polyclonal antibodies, single chain anti-TAT antibodies, and fragments of anti-TAT antibodies (see below) as long as they exhibit the desired biological or immunological activity. The term “immunoglobulin” (Ig) is used interchangeable with antibody herein.

An “isolated antibody” is one which has been identified and separated and/or recovered from a component of its natural environment. Contaminant components of its natural environment are materials which would interfere with diagnostic or therapeutic uses for the antibody, and may include enzymes, hormones, and other proteinaceous or nonproteinaceous solutes. In preferred embodiments, the antibody will be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

The basic 4-chain antibody unit is a heterotetrameric glycoprotein composed of two identical light (L) chains and two identical heavy (H) chains (an IgM antibody consists of 5 of the basic heterotetramer unit along with an additional polypeptide called J chain, and therefore contain 10 antigen binding sites, while secreted IgA antibodies can polymerize to form polyvalent assemblages comprising 2-5 of the basic 4-chain units along with J chain). In the case of IgGs, the 4-chain unit is generally about 150,000 daltons. Each L chain is linked to a H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable domain (V_{H}) followed by three constant domains (C_{H}) for each of the α and γ chains and four C_{H} domains for μ and ε isotypes. Each L chain has at the N-terminus, a variable domain (V_{L}) followed by a constant domain (C_{L}) at its other end. The V_{L} is aligned with the V_{H} and the C_{L} is aligned with the first constant domain of the heavy chain (C_{H}1). Particular amino acid residues are believed to form an interface between the light chain and heavy chain variable domains. The pairing of a V_{H} and V_{L} together forms a single antigen-binding site. For the structure and properties of the different classes of antibodies, see, e.g., Basic and Clinical Immunology, 8th edition, Daniel P. Stites, Abba I. Terr and Tristram G. Parslow (eds.), Appleton & Lange, Norwalk, Conn., 1994, page 71 and Chapter 6.

The L chain from any vertebrate species can be assigned to one of two clearly distinct types, called kappa and lambda, based on the amino acid sequences of their constant domains. Depending on the amino acid sequence of the constant domain of their heavy chains (C_{H}), immunoglobulins can be assigned to different classes or isotypes. There are five classes of immunoglobulins: IgA, IgD, IgE, IgG, and IgM, having heavy chains designated α, δ, ε, γ, and μ, respectively. They and a classes are further divided into subclasses on the basis of relatively minor differences in C_{H} sequence and function, e.g., humans express the following subclasses: IgG1, IgG2, IgG3, IgG4, IgA1, and IgA2.

The term “variable” refers to the fact that certain segments of the variable domains differ extensively in sequence among antibodies. The V domain mediates antigen binding and define specificity of a particular antibody for its particular antigen. However, the variability is not evenly distributed across the 110-amino acid span of the variable domains. Instead, the V regions consist of relatively invariant stretches called framework regions (FRs) of 15-30 amino acids separated by shorter regions of extreme variability called “hypervariable regions” that are each 9-12 amino acids long. The variable domains of native heavy and light chains each comprise four FRs, largely adopting a β-sheet configuration, connected by three hypervariable regions, which form loops connecting, and in some cases forming part of, the β-sheet structure. The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, Md. (1991)). The constant domains are not involved directly in binding an antibody to an antigen, but exhibit various effector functions, such as participation of the antibody in antibody dependent cellular cytotoxicity (ADCC).

The term “hypervariable region” when used herein refers to the amino acid residues of an antibody which are responsible for antigen-binding. The hypervariable region generally comprises amino acid residues from a “complementarity determining region” or “CDR” (e.g. around about residues 24-34 (L1), 50-56 (L2) and 89-97 (L3) in the V_{L}, and around about 1-35 (H1), 50-65 (H2) and 95-102 (H3) in the V_{H}; Kabat et al., Sequences of Proteins of Immunological Interest, 5th Ed. Public Health Service, National Institutes of Health, Bethesda, Md. (1991)) and/or those residues from a “hypervariable loop” (e.g. residues 26-32 (L1), 50-52 (L2) and 91-96 (L3) in the V_{L}, and 26-32 (H1), 53-55 (H2) and 96-101 (H3) in the V_{H}; Chothia and Lesk J. Mol. Biol. 196:901-917 (1987)).

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to polyclonal antibody preparations which include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” is not to be construed as requiring production of the antibody by any particular method. For example, the monoclonal antibodies useful in the present invention may be prepared by the hybridoma methodology first described by Kohler et al., Nature, 256:495 (1975), or may be made using recombinant DNA methods in bacterial, eukaryotic animal or plant cells (see, e.g., U.S. Pat. No. 4,816,567). The “monoclonal antibodies” may also be isolated from phage antibody libraries using the techniques described in Clackson et al., Nature, 352:624-628 (1991) and Marks et al., J. Mol. Biol., 222:581-597 (1991), for example.

The monoclonal antibodies herein include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see U.S. Pat. No. 4,816,567; and Morrison et al., Proc. Natl. Acad. Sci. USA, 81:6851-6855 (1984)). Chimeric antibodies of interest herein include “primatized” antibodies comprising variable domain antigen-binding sequences derived from a non-human primate (e.g. Old World Monkey, Ape etc), and human constant region sequences.

An “intact” antibody is one which comprises an antigen-binding site as well as a C_{L} and at least heavy chain constant domains, C_{H}1, C_{H}2 and C_{H}3. The constant domains may be native sequence constant domains (e.g. human native sequence constant domains) or amino acid sequence variant thereof. Preferably, the intact antibody has one or more effector functions.

“Antibody fragments” comprise a portion of an intact antibody, preferably the antigen binding or variable region of the intact antibody. Examples of antibody fragments include Fab, Fab′, F(ab′)₂, and Fv fragments; diabodies; linear antibodies (see U.S. Pat. No. 5,641,870, Example 2; Zapata et al., Protein Eng. 8(10): 1057-1062 [1995]); single-chain antibody molecules; and multispecific antibodies formed from antibody fragments.

Papain digestion of antibodies produces two identical antigen-binding fragments, called “Fab” fragments, and a residual “Fc” fragment, a designation reflecting the ability to crystallize readily. The Fab fragment consists of an entire L chain along with the variable region domain of the H chain (V_{H}), and the first constant domain of one heavy chain (C_{H}1). Each Fab fragment is monovalent with respect to antigen binding, i.e., it has a single antigen-binding site. Pepsin treatment of an antibody yields a single large F(ab′)₂ fragment which roughly corresponds to two disulfide linked Fab fragments having divalent antigen-binding activity and is still capable of cross-linking antigen. Fab′ fragments differ from Fab fragments by having additional few residues at the carboxy terminus of the C_{H}1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

The Fc fragment comprises the carboxy-terminal portions of both H chains held together by disulfides. The effector functions of antibodies are determined by sequences in the Pc region, which region is also the part recognized by Pc receptors (FcR) found on certain types of cells.

“Fv” is the minimum antibody fragment which contains a complete antigen-recognition and -binding site. This fragment consists of a dimer of one heavy- and one light-chain variable region domain in tight, non-covalent association. From the folding of these two domains emanate six hypervariable loops (3 loops each from the H and L chain) that contribute the amino acid residues for antigen binding and confer antigen binding specificity to the antibody. However, even a single variable domain (or half of an Fv comprising only three CDRs specific for an antigen) has the ability to recognize and bind antigen, although at a lower affinity than the entire binding site.

“Single-chain Fv” also abbreviated as “sFv” or “scFv” are antibody fragments that comprise the V_{H} and V_{L} antibody domains connected into a single polypeptide chain. Preferably, the sFv polypeptide further comprises a polypeptide linker between the V_{H} and V_{L} domains which enables the sFv to form the desired structure for antigen binding. For a review of sFv, see Pluckthun in The Pharmacology of Monoclonal Antibodies, vol. 113, Rosenburg and Moore eds., Springer-Verlag, New York, pp. 269-315 (1994); Borrebaeck 1995, infra.

The term “diabodies” refers to small antibody fragments prepared by constructing sFv fragments (see preceding paragraph) with short linkers (about 5-10 residues) between the V_{H} and V_{L} domains such that inter-chain but not intra-chain pairing of the V domains is achieved, resulting in a bivalent fragment, i.e., fragment having two antigen-binding sites. Bispecific diabodies are heterodimers of two “crossover” sFv fragments in which the V_{H} and V_{L} domains of the two antibodies are present on different polypeptide chains. Diabodies are described more fully in, for example, EP 404,097; WO 93/11161; and Hollinger et al., Proc. Natl. Acad. Sci. USA, 90:6444-6448 (1993).

“Humanized” forms of non-human (e.g., rodent) antibodies are chimeric antibodies that contain minimal sequence derived from the non-human antibody. For the most part, humanized antibodies are human immunoglobulins (recipient antibody) in which residues from a hypervariable region of the recipient are replaced by residues from a hypervariable region of a non-human species (donor antibody) such as mouse, rat, rabbit or non-human primate having the desired antibody specificity, affinity, and capability. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992).

A “species-dependent antibody,” e.g., a mammalian anti-human IgE antibody, is an antibody which has a stronger binding affinity for an antigen from a first mammalian species than it has for a homologue of that antigen from a second mammalian species. Normally, the species-dependent antibody “bind specifically” to a human antigen (i.e., has a binding affinity (Kd) value of no more than about 1×10⁻⁷ M, preferably no more than about 1×10⁻⁸ and most preferably no more than about 1×10⁻⁹ M) but has a binding affinity for a homologue of the antigen from a second non-human mammalian species which is at least about 50 fold, or at least about 500 fold, or at least about 1000 fold, weaker than its binding affinity for the human antigen. The species-dependent antibody can be of any of the various types of antibodies as defined above, but preferably is a humanized or human antibody.

A “TAT binding oligopeptide” is an oligopeptide that binds, preferably specifically, to a TAT polypeptide as described herein. TAT binding oligopeptides may be chemically synthesized using known oligopeptide synthesis methodology or may be prepared and purified using recombinant technology. TAT binding oligopeptides are usually at least about 5 amino acids in length, alternatively at least about 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 amino acids in length or more, wherein such oligopeptides that are capable of binding, preferably specifically, to a TAT polypeptide as described herein. TAT binding oligopeptides may be identified without undue experimentation using well known techniques. In this regard, it is noted that techniques for screening oligopeptide libraries for oligopeptides that are capable of specifically binding to a polypeptide target are well known in the art (see, e.g., U.S. Pat. Nos. 5,556,762, 5,750,373, 4,708,871, 4,833,092, 5,223,409, 5,403,484, 5,571,689, 5,663,143; PCT Publication Nos. WO 84/03506 and WO84/03564; Geysen et al., Proc. Natl. Acad. Sci. U.S.A., 81:3998-4002 (1984); Geysen et al., Proc. Natl. Acad. Sci. U.S.A., 82:178-182 (1985); Geysen et al., in Synthetic Peptides as Antigens, 130-149 (1986); Geysen et al., J. Immunol. Meth., 102:259-274 (1987); Schoofs et al., J. Immunol., 140:611-616 (1988), Cwirla, S. E. et al. (1990) Proc. Natl. Acad. Sci. USA, 87:6378; Lowman, H. B. et al. (1991) Biochemistry, 30:10832; Clackson, T. et al. (1991) Nature, 352: 624; Marks, J. D. et al. (1991), J. Mol. Biol., 222:581; Kang, A. S. et al. (1991) Proc. Natl. Acad. Sci. USA, 88:8363, and Smith, G. P. (1991) Current Opin. Biotechnol., 2:668).

A “TAT binding organic molecule” is an organic molecule other than an oligopeptide or antibody as defined herein that binds, preferably specifically, to a TAT polypeptide as described herein. TAT binding organic molecules may be identified and chemically synthesized using known methodology (see, e.g., PCT Publication Nos. WO00/00823 and WO00/39585). TAT binding organic molecules are usually less than about 2000 daltons in size, alternatively less than about 1500, 750, 500, 250 or 200 daltons in size, wherein such organic molecules that are capable of binding, preferably specifically, to a TAT polypeptide as described herein may be identified without undue experimentation using well known techniques. In this regard, it is noted that techniques for screening organic molecule libraries for molecules that are capable of binding to a polypeptide target are well known in the art (see, e.g., PCT Publication Nos. WO00/00823 and WO00/39585).

An antibody, oligopeptide or other organic molecule “which binds” an antigen of interest, e.g. a tumor-associated polypeptide antigen target, is one that binds the antigen with sufficient affinity such that the antibody, oligopeptide or other organic molecule is useful as a diagnostic and/or therapeutic agent in targeting a cell or tissue expressing the antigen, and does not significantly cross-react with other proteins. In such embodiments, the extent of binding of the antibody, oligopeptide or other organic molecule to a “non-target” protein will be less than about 10% of the binding of the antibody, oligopeptide or other organic molecule to its particular target protein as determined by fluorescence activated cell sorting (FACS) analysis or radioimmunoprecipitation (RIA). With regard to the binding of an antibody, oligopeptide or other organic molecule to a target molecule, the term “specific binding” or “specifically binds to” or is “specific for” a particular polypeptide or an epitope on a particular polypeptide target means binding that is measurably different from a non-specific interaction. Specific binding can be measured, for example, by determining binding of a molecule compared to binding of a control molecule, which generally is a molecule of similar structure that does not have binding activity. For example, specific binding can be determined by competition with a control molecule that is similar to the target, for example, an excess of non-labeled target. In this case, specific binding is indicated if the binding of the labeled target to a probe is competitively inhibited by excess unlabeled target. The term “specific binding” or “specifically binds to” or is “specific for” a particular polypeptide or an epitope on a particular polypeptide target as used herein can be exhibited, for example, by a molecule having a Kd for the target of at least about 10⁻⁴ M, alternatively at least about 10⁻⁵ M, alternatively at least about 10⁻⁶ M, alternatively at least about 10⁻⁷ M, alternatively at least about 10⁻⁸ M, alternatively at least about 10⁻⁹ M, alternatively at least about 10⁻¹⁰ M, alternatively at least about 10⁻¹¹ M, alternatively at least about 10⁻¹² M, or greater. In one embodiment, the term “specific binding” refers to binding where a molecule binds to a particular polypeptide or epitope on a particular polypeptide without substantially binding to any other polypeptide or polypeptide epitope.

An antibody, oligopeptide or other organic molecule that “inhibits the growth of tumor cells expressing a TAT polypeptide” or a “growth inhibitory” antibody, oligopeptide or other organic molecule is one which results in measurable growth inhibition of cancer cells expressing or overexpressing the appropriate TAT polypeptide. The TAT polypeptide may be a transmembrane polypeptide expressed on the surface of a cancer cell or may be a polypeptide that is produced and secreted by a cancer cell. Preferred growth inhibitory anti-TAT antibodies, oligopeptides or organic molecules inhibit growth of TAT-expressing tumor cells by greater than 20%, preferably from about 20% to about 50%, and even more preferably, by greater than 50% (e.g., from about 50% to about 100%) as compared to the appropriate control, the control typically being tumor cells not treated with the antibody, oligopeptide or other organic molecule being tested. In one embodiment, growth inhibition can be measured at an antibody concentration of about 0.1 to 30 μg/ml or about 0.5 nM to 200 nM in cell culture, where the growth inhibition is determined 1-10 days after exposure of the tumor cells to the antibody. Growth inhibition of tumor cells in vivo can be determined in various ways such as is described in the Experimental Examples section below. The antibody is growth inhibitory in vivo if administration of the anti-TAT antibody at about 1 μg/kg to about 100 mg/kg body weight results in reduction in tumor size or tumor cell proliferation within about 5 days to 3 months from the first administration of the antibody, preferably within about 5 to 30 days.

An antibody, oligopeptide or other organic molecule which “induces apoptosis” is one which induces programmed cell death as determined by binding of annexin V, fragmentation of DNA, cell shrinkage, dilation of endoplasmic reticulum, cell fragmentation, and/or formation of membrane vesicles (called apoptotic bodies). The cell is usually one which overexpresses a TAT polypeptide. Preferably the cell is a tumor cell, e.g., a prostate, breast, ovarian, stomach, endometrial, lung, kidney, colon, bladder cell. Various methods are available for evaluating the cellular events associated with apoptosis. For example, phosphatidyl serine (PS) translocation can be measured by annexin binding; DNA fragmentation can be evaluated through DNA laddering; and nuclear/chromatin condensation along with DNA fragmentation can be evaluated by any increase in hypodiploid cells. Preferably, the antibody, oligopeptide or other organic molecule which induces apoptosis is one which results in about 2 to 50 fold, preferably about 5 to 50 fold, and most preferably about 10 to 50 fold, induction of annexin binding relative to untreated cell in an annexin binding assay.

Antibody “effector functions” refer to those biological activities attributable to the Fc region (a native sequence Fc region or amino acid sequence variant Fc region) of an antibody, and vary with the antibody isotype. Examples of antibody effector functions include: C1q binding and complement dependent cytotoxicity; Fc receptor binding; antibody-dependent cell-mediated cytotoxicity (ADCC); phagocytosis; down regulation of cell surface receptors (e.g., B cell receptor); and B cell activation.

“Antibody-dependent cell-mediated cytotoxicity” or “ADCC” refers to a form of cytotoxicity in which secreted Ig bound onto Fc receptors (FcRs) present on certain cytotoxic cells (e.g., Natural Killer (NK) cells, neutrophils, and macrophages) enable these cytotoxic effector cells to bind specifically to an antigen-bearing target cell and subsequently kill the target cell with cytotoxins. The antibodies “arm” the cytotoxic cells and are absolutely required for such killing. The primary cells for mediating ADCC, NK cells, express FcγRIII only, whereas monocytes express PcγRI, FcγRII and FcγRIII. FcR expression on hematopoietic cells is summarized in Table 3 on page 464 of Ravetch and Kinet, Annu. Rev. Immunol. 9:457-92 (1991). To assess ADCC activity of a molecule of interest, an in vitro ADCC assay, such as that described in U.S. Pat. No. 5,500,362 or 5,821,337 may be performed. Useful effector cells for such assays include peripheral blood mononuclear cells (PBMC) and Natural Killer (NK) cells. Alternatively, or additionally, ADCC activity of the molecule of interest may be assessed in vivo, e.g., in a animal model such as that disclosed in Clynes et al. (USA) 95:652-656 (1998).

“Fc receptor” or “FcR” describes a receptor that binds to the Fc region of an antibody. The preferred FcR is a native sequence human FcR. Moreover, a preferred FcR is one which binds an IgG antibody (a gamma receptor) and includes receptors of the FcγRI, FcγRII and FcγRIII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain. (see review M. in Daëron, Annu. Rev. Immunol. 15:203-234 (1997)). FcRs are reviewed in Ravetch and Kinet, Annu. Rev. Immunol. 9:457-492 (1991); Capel et al., Immunomethods 4:25-34 (1994); and de Haas et al., J. Lab. Clin. Med. 126:330-41 (1995). Other FcRs, including those to be identified in the future, are encompassed by the term “FcR” herein. The term also includes the neonatal receptor, FcRn, which is responsible for the transfer of maternal IgGs to the fetus (Guyer et al., J. Immunol. 117:587 (1976) and Kim et al., J. Immunol. 24:249 (1994)).

“Human effector cells” are leukocytes which express one or more FcRs and perform effector functions. Preferably, the cells express at least FcγRIII and perform ADCC effector function. Examples of human leukocytes which mediate ADCC include peripheral blood mononuclear cells (PBMC), natural killer (NK) cells, monocytes, cytotoxic T cells and neutrophils; with PBMCs and NK cells being preferred. The effector cells may be isolated from a native source, e.g., from blood.

“Complement dependent cytotoxicity” or “CDC” refers to the lysis of a target cell in the presence of complement. Activation of the classical complement pathway is initiated by the binding of the first component of the complement system (C1q) to antibodies (of the appropriate subclass) which are bound to their cognate antigen. To assess complement activation, a CDC assay, e.g., as described in Gazzano-Santoro et al., J. Immunol. Methods 202:163 (1996), may be performed.

The terms “cancer” and “cancerous” refer to or describe the physiological condition in mammals that is typically characterized by unregulated cell growth. Examples of cancer include, but are not limited to, carcinoma, lymphoma, blastoma, sarcoma, and leukemia or lymphoid malignancies. More particular examples of such cancers include squamous cell cancer (e.g., epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, cancer of the urinary tract, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, melanoma, multiple myeloma and B-cell lymphoma, brain, as well as head and neck cancer, and associated metastases.

The terms “cell proliferative disorder” and “proliferative disorder” refer to disorders that are associated with some degree of abnormal cell proliferation. In one embodiment, the cell proliferative disorder is cancer.

“Tumor”, as used herein, refers to all neoplastic cell growth and proliferation, whether malignant or benign, and all pre-cancerous and cancerous cells and tissues.

An antibody, oligopeptide or other organic molecule which “induces cell death” is one which causes a viable cell to become nonviable. The cell is one which expresses a TAT polypeptide, preferably a cell that overexpresses a TAT polypeptide as compared to a normal cell of the same tissue type. The TAT polypeptide may be a transmembrane polypeptide expressed on the surface of a cancer cell or may be a polypeptide that is produced and secreted by a cancer cell. Preferably, the cell is a cancer cell, e.g., a breast, ovarian, stomach, endometrial, salivary gland, lung, kidney, colon, thyroid, pancreatic or bladder cell. Cell death in vitro may be determined in the absence of complement and immune effector cells to distinguish cell death induced by antibody-dependent cell-mediated cytotoxicity (ADCC) or complement dependent cytotoxicity (CDC). Thus, the assay for cell death may be performed using heat inactivated serum (i.e., in the absence of complement) and in the absence of immune effector cells. To determine whether the antibody, oligopeptide or other organic molecule is able to induce cell death, loss of membrane integrity as evaluated by uptake of propidium iodide (PI), trypan blue (see Moore et al. Cytotechnology 17:1-11 (1995)) or 7AAD can be assessed relative to untreated cells. Preferred cell death-inducing antibodies, oligopeptides or other organic molecules are those which induce PI uptake in the PI uptake assay in BT474 cells.

A “TAT-expressing cell” is a cell which expresses an endogenous or transfected TAT polypeptide either on the cell surface or in a secreted form. A “TAT-expressing cancer” is a cancer comprising cells that have a TAT polypeptide present on the cell surface or that produce and secrete a TAT polypeptide. A “TAT-expressing cancer” optionally produces sufficient levels of TAT polypeptide on the surface of cells thereof, such that an anti-TAT antibody, oligopeptide ot other organic molecule can bind thereto and have a therapeutic effect with respect to the cancer. In another embodiment, a “TAT-expressing cancer” optionally produces and secretes sufficient levels of TAT polypeptide, such that an anti-TAT antibody, oligopeptide ot other organic molecule antagonist can bind thereto and have a therapeutic effect with respect to the cancer. With regard to the latter, the antagonist may be an antisense oligonucleotide which reduces, inhibits or prevents production and secretion of the secreted TAT polypeptide by tumor cells. A cancer which “overexpresses” a TAT polypeptide is one which has significantly higher levels of TAT polypeptide at the cell surface thereof, or produces and secretes, compared to a noncancerous cell of the same tissue type. Such overexpression may be caused by gene amplification or by increased transcription or translation. TAT polypeptide overexpression may be determined in a diagnostic or prognostic assay by evaluating increased levels of the TAT protein present on the surface of a cell, or secreted by the cell (e.g., via an immunohistochemistry assay using anti-TAT antibodies prepared against an isolated TAT polypeptide which may be prepared using recombinant DNA technology from an isolated nucleic acid encoding the TAT polypeptide; FACS analysis, etc.). Alternatively, or additionally, one may measure levels of TAT polypeptide-encoding nucleic acid or mRNA in the cell, e.g., via fluorescent in situ hybridization using a nucleic acid based probe corresponding to a TAT-encoding nucleic acid or the complement thereof; (FISH; see WO98/45479 published October, 1998), Southern blotting, Northern blotting, or polymerase chain reaction (PCR) techniques, such as real time quantitative PCR (RT-PCR). One may also study TAT polypeptide overexpression by measuring shed antigen in a biological fluid such as serum, e.g, using antibody-based assays (see also, e.g., U.S. Pat. No. 4,933,294 issued Jun. 12, 1990; WO91/05264 published Apr. 18, 1991; U.S. Pat. No. 5,401,638 issued Mar. 28, 1995; and Sias et al., J. Immunol. Methods 132:73-80 (1990)). Aside from the above assays, various in vivo assays are available to the skilled practitioner. For example, one may expose cells within the body of the patient to an antibody which is optionally labeled with a detectable label, e.g., a radioactive isotope, and binding of the antibody to cells in the patient can be evaluated, e.g., by external scanning for radioactivity or by analyzing a biopsy taken from a patient previously exposed to the antibody.

As used herein, the term “immunoadhesin” designates antibody-like molecules which combine the binding specificity of a heterologous protein (an “adhesin”) with the effector functions of immunoglobulin constant domains. Structurally, the immunoadhesins comprise a fusion of an amino acid sequence with the desired binding specificity which is other than the antigen recognition and binding site of an antibody (i.e., is “heterologous”), and an immunoglobulin constant domain sequence. The adhesin part of an immunoadhesin molecule typically is a contiguous amino acid sequence comprising at least the binding site of a receptor or a ligand. The immunoglobulin constant domain sequence in the immunoadhesin may be obtained from any immunoglobulin, such as IgG-1, IgG-2, IgG-3, or IgG-4 subtypes, IgA (including IgA-1 and IgA-2), IgE, IgD or IgM.

The word “label” when used herein refers to a detectable compound or composition which is conjugated directly or indirectly to the antibody, oligopeptide or other organic molecule so as to generate a “labeled” antibody, oligopeptide or other organic molecule. The label may be detectable by itself (e.g. radioisotope labels or fluorescent labels) or, in the case of an enzymatic label, may catalyze chemical alteration of a substrate compound or composition which is detectable.

The term “cytotoxic agent” as used herein refers to a substance that inhibits or prevents the function of cells and/or causes destruction of cells. The term is intended to include radioactive isotopes (e.g., At²¹¹, I¹³¹, I¹²⁵, Y⁹⁰, Re¹⁸⁶, Re¹⁸⁸, Sm¹⁵³, Bi²¹², P³² and radioactive isotopes of Lu), chemotherapeutic agents e.g. methotrexate, adriamicin, vinca alkaloids (vincristine, vinblastine, etoposide), doxorubicin, melphalan, mitomycin C, chlorambucil, daunorubicin or other intercalating agents, enzymes and fragments thereof such as nucleolytic enzymes, antibiotics, and toxins such as small molecule toxins or enzymatically active toxins of bacterial, fungal, plant or animal origin, including fragments and/or variants thereof, and the various antitumor or anticancer agents disclosed below. Other cytotoxic agents are described below. A tumoricidal agent causes destruction of tumor cells.

A “growth inhibitory agent” when used herein refers to a compound or composition which inhibits growth of a cell, especially a TAT-expressing cancer cell, either in vitro or in vivo. Thus, the growth inhibitory agent may be one which significantly reduces the percentage of TAT-expressing cells in S phase. Examples of growth inhibitory agents include agents that block cell cycle progression (at a place other than S phase), such as agents that induce G1 arrest and M-phase arrest. Classical M-phase blockers include the vincas (vincristine and vinblastine), taxanes, and topoisomerase II inhibitors such as doxorubicin, epirubicin, daunorubicin, etoposide, and bleomycin. Those agents that arrest G1 also spill over into S-phase arrest, for example, DNA alkylating agents such as tamoxifen, prednisone, dacarbazine, mechlorethamine, cisplatin, methotrexate, 5-fluorouracil, and ara-C. Further information can be found in The Molecular Basis of Cancer, Mendelsohn and Israel, eds., Chapter 1, entitled “Cell cycle regulation, oncogenes, and antineoplastic drugs” by Murakami et al. (WB Saunders: Philadelphia, 1995), especially p. 13. The taxanes (paclitaxel and docetaxel) are anticancer drugs both derived from the yew tree. Docetaxel (TAXOTERE®, Rhone-Poulenc Rorer), derived from the European yew, is a semisynthetic analogue of paclitaxel (TAXOL®, Bristol-Myers Squibb). Paclitaxel and docetaxel promote the assembly of microtubules from tubulin dimers and stabilize microtubules by preventing depolymerization, which results in the inhibition of mitosis in cells.

“Doxorubicin” is an anthracycline antibiotic. The full chemical name of doxorubicin is (8S-cis)-10-[(3-amino-2,3,6-trideoxya-α-L-lyxo-hexapyranosyl)oxy]-7,8,9,10-tetrahydro-6,8,11-trihydroxy-8-(hydroxyacetyl)-1-methoxy-5,12-naphthacenedione.

The term “cytokine” is a generic term for proteins released by one cell population which act on another cell as intercellular mediators. Examples of such cytokines are lymphokines, monokines, and traditional polypeptide hormones. Included among the cytokines are growth hormone such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinzing hormone (LH); hepatic growth factor; fibroblast growth factor; prolactin; placental lactogen; tumor necrosis factor-α and -β; mullerian-inhibiting substance; mouse gonadotropin-associated peptide; inhibin; activin; vascular endothelial growth factor; integrin; thrombopoietin (TPO); nerve growth factors such as NGF-β; platelet-growth factor; transforming growth factors (TGFs) such as TGF-α and TGF-β; insulin-like growth factor-I and -II; erythropoietin (EPO); osteoinductive factors; interferons such as interferon -α, -β, and -γ; colony stimulating factors (CSFs) such as macrophage-CSF (M-CSF); granulocyte-macrophage-CSF (GM-CSF); and granulocyte-CSF (G-CSF); interleukins (ILs) such as IL-1, IL-1a, IL-2, IL-3, IL-4, IL-5, IL-6, IL-7, IL-8, IL-9, IL-11, IL-12; a tumor necrosis factor such as TNF-α or TNF-β; and other polypeptide factors including LIP and kit ligand (KL). As used herein, the term cytokine includes proteins from natural sources or from recombinant cell culture and biologically active equivalents of the native sequence cytokines.

The term “package insert” is used to refer to instructions customarily included in commercial packages of therapeutic products, that contain information about the indications, usage, dosage, administration, contraindications and/or warnings concerning the use of such therapeutic products.

TABLE 2

| | | |
|---|---|---|
| TAT | XXXXXXXXXXXXXXX | (Length = 15 amino acids) |
| Comparison | XXXXXYYYYYYY | (Length = 12 amino acids) |
| Protein | | |

% amino acid sequence identity = (the number of identically matching amino acid residues between the two polypeptide sequences as determined by ALIGN-2) divided by (the total number of amino acid residues of the TAT polypeptide) = 5 divided by 15 = 33.3%

TABLE 3

| | | |
|---|---|---|
| TAT | XXXXXXXXXX | (Length = 10 amino acids) |
| Comparison | XXXXXYYYYYYZZYZ | (Length = 15 amino acids) |
| Protein | | |

% amino acid sequence identity = (the number of identically matching amino acid residues between the two polypeptide sequences as determined by ALIGN-2) divided by (the total number of amino acid residues of the TAT polypeptide) = 5 divided by 10 = 50%

TABLE 4

| | | |
|---|---|---|
| TAT-DNA | NNNNNNNNNNNNNN | (Length = 14 nucleotides) |
| Comparison | NNNNNNLLLLLLLLLL | (Length = 16 nucleotides) |
| DNA | | |

% nucleic acid sequence identity = (the number of identically matching nucleotides between the two nucleic acid sequences as determined by ALIGN-2) divided by (the total number of nucleotides of the TAT-DNA nucleic acid sequence) = 6 divided by 14 = 42.9%

TABLE 5

| | | |
|---|---|---|
| TAT-DNA | NNNNNNNNNNNN | (Length = 12 nucleotides) |
| Comparison DNA | NNNNLLLVV | (Length = 9 nucleotides) |

% nucleic acid sequence identity = (the number of identically matching nucleotides between the two nucleic acid sequences as determined by ALIGN-2) divided by (the total number of nucleotides of the TAT-DNA nucleic acid sequence) = 4 divided by 12 = 33.3%

II. Compositions and Methods of the Invention

A. Anti-TAT Antibodies

In one embodiment, the present invention provides anti-TAT antibodies which may find use herein as therapeutic and/or diagnostic agents. Exemplary antibodies include polyclonal, monoclonal, humanized, bispecific, and heteroconjugate antibodies.

1. Polyclonal Antibodies

Polyclonal antibodies are preferably raised in animals by multiple subcutaneous (sc) or intraperitoneal (ip) injections of the relevant antigen and an adjuvant. It may be useful to conjugate the relevant antigen (especially when synthetic peptides are used) to a protein that is immunogenic in the species to be immunized. For example, the antigen can be conjugated to keyhole limpet hemocyanin (KLH), serum albumin, bovine thyroglobulin, or soybean trypsin inhibitor, using a bifunctional or derivatizing agent, e.g., maleimidobenzoyl sulfosuccinimide ester (conjugation through cysteine residues), N-hydroxysuccinimide (through lysine residues), glutaraldehyde, succinic anhydride, SOCl₂, or R¹N═C═NR, where R and R¹ are different alkyl groups.

Animals are immunized against the antigen, immunogenic conjugates, or derivatives by combining, e.g., 100 μg or 5 μg of the protein or conjugate (for rabbits or mice, respectively) with 3 volumes of Freund's complete adjuvant and injecting the solution intradermally at multiple sites. One month later, the animals are boosted with ⅕ to 1/10 the original amount of peptide or conjugate in Freund's complete adjuvant by subcutaneous injection at multiple sites. Seven to 14 days later, the animals are bled and the serum is assayed for antibody titer. Animals are boosted until the titer plateaus. Conjugates also can be made in recombinant cell culture as protein fusions. Also, aggregating agents such as alum are suitably used to enhance the immune response.

2. Monoclonal Antibodies

Monoclonal antibodies may be made using the hybridoma method first described by Kohler et al., Nature, 256:495 (1975), or may be made by recombinant DNA methods (U.S. Pat. No. 4,816,567).

In the hybridoma method, a mouse or other appropriate host animal, such as a hamster, is immunized as described above to elicit lymphocytes that produce or are capable of producing antibodies that will specifically bind to the protein used for immunization. Alternatively, lymphocytes may be immunized in vitro. After immunization, lymphocytes are isolated and then fused with a myeloma cell line using a suitable fusing agent, such as polyethylene glycol, to form a hybridoma cell (Goding, Monoclonal Antibodies: Principles and Practice, pp. 59-103 (Academic Press, 1986)).

The hybridoma cells thus prepared are seeded and grown in a suitable culture medium which medium preferably contains one or more substances that inhibit the growth or survival of the unfused, parental myeloma cells (also referred to as fusion partner). For example, if the parental myeloma cells lack the enzyme hypoxanthine guanine phosphoribosyl transferase (HGPRT or HPRT), the selective culture medium for the hybridomas typically will include hypoxanthine, aminopterin, and thymidine (HAT medium), which substances prevent the growth of HGPRT-deficient cells.

Preferred fusion partner myeloma cells are those that fuse efficiently, support stable high-level production of antibody by the selected antibody-producing cells, and are sensitive to a selective medium that selects against the unfused parental cells. Preferred myeloma cell lines are murine myeloma lines, such as those derived from MOPC-21 and MPC-11 mouse tumors available from the Salk Institute Cell Distribution Center, San Diego, Calif. USA, and SP-2 and derivatives e.g., X63-Ag8653 cells available from the American Type Culture Collection, Manassas, Va., USA. Human myeloma and mouse-human heteromyeloma cell lines also have been described for the production of human monoclonal antibodies (Kozbor, J. Immunol., 133:3001 (1984); and Brodeur et al., Monoclonal Antibody Production Techniques and Applications, pp. 51-63 (Marcel Dekker, Inc., New York, 1987)).

Culture medium in which hybridoma cells are growing is assayed for production of monoclonal antibodies directed against the antigen. Preferably, the binding specificity of monoclonal antibodies produced by hybridoma cells is determined by immunoprecipitation or by an in vitro binding assay, such as radioimmunoassay (RIA) or enzyme-linked immunosorbent assay (ELISA).

The binding affinity of the monoclonal antibody can, for example, be determined by the Scatchard analysis described in Munson et al., Anal. Biochem., 107:220 (1980).

Once hybridoma cells that produce antibodies of the desired specificity, affinity, and/or activity are identified, the clones may be subcloned by limiting dilution procedures and grown by standard methods (Goding, Monoclonal Antibodies: Principles and Practice, pp. 59-103 (Academic Press, 1986)). Suitable culture media for this purpose include, for example, D-MEM or RPMI-1640 medium. In addition, the hybridoma cells may be grown in vivo as ascites tumors in an animal e.g, by i.p. injection of the cells into mice.

The monoclonal antibodies secreted by the subclones are suitably separated from the culture medium, ascites fluid, or serum by conventional antibody purification procedures such as, for example, affinity chromatography (e.g., using protein A or protein G-Sepharose) or ion-exchange chromatography, hydroxylapatite chromatography, gel electrophoresis, dialysis, etc.

DNA encoding the monoclonal antibodies is readily isolated and sequenced using conventional procedures (e.g., by using oligonucleotide probes that are capable of binding specifically to genes encoding the heavy and light chains of murine antibodies). The hybridoma cells serve as a preferred source of such DNA. Once isolated, the DNA may be placed into expression vectors, which are then transfected into host cells such as E. coli cells, simian COS cells, Chinese Hamster Ovary (CHO) cells, or myeloma cells that do not otherwise produce antibody protein, to obtain the synthesis of monoclonal antibodies in the recombinant host cells. Review articles on recombinant expression in bacteria of DNA encoding the antibody include Skerra et al., Curr. Opinion in Immunol., 5:256-262 (1993) and Plückthun, Immunol. Revs. 130:151-188 (1992).

In a further embodiment, monoclonal antibodies or antibody fragments can be isolated from antibody phage libraries generated using the techniques described in McCafferty et al., Nature, 348:552-554 (1990). Clackson et al., Nature, 352:624-628 (1991) and Marks et al., J. Mol. Biol., 222:581-597 (1991) describe the isolation of murine and human antibodies, respectively, using phage libraries. Subsequent publications describe the production of high affinity (nM range) human antibodies by chain shuffling (Marks et al., Bio/Technology, 10:779-783 (1992)), as well as combinatorial infection and in vivo recombination as a strategy for constructing very large phage libraries (Waterhouse et al., Nuc. Acids. Res. 21:2265-2266 (1993)). Thus, these techniques are viable alternatives to traditional monoclonal antibody hybridoma techniques for isolation of monoclonal antibodies.

The DNA that encodes the antibody may be modified to produce chimeric or fusion antibody polypeptides, for example, by substituting human heavy chain and light chain constant domain (C_{H} and C_{L}) sequences for the homologous murine sequences (U.S. Pat. No. 4,816,567; and Morrison, et al., Proc. Natl. Acad. Sci. USA, 81:6851 (1984)), or by fusing the immunoglobulin coding sequence with all or part of the coding sequence for a non-immunoglobulin polypeptide (heterologous polypeptide). The non-immunoglobulin polypeptide sequences can substitute for the constant domains of an antibody, or they are substituted for the variable domains of one antigen-combining site of an antibody to create a chimeric bivalent antibody comprising one antigen-combining site having specificity for an antigen and another antigen-combining site having specificity for a different antigen.

3. Human and Humanized Antibodies

The anti-TAT antibodies of the invention may further comprise humanized antibodies or human antibodies. Humanized forms of non-human (e.g., murine) antibodies are chimeric immunoglobulins, immunoglobulin chains or fragments thereof (such as Pv, Fab, Fab′, F(ab′)₂ or other antigen-binding subsequences of antibodies) which contain minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin [Jones et al., Nature, 321:522-525 (1986); Riechmann et al., Nature, 332:323-329 (1988); and Presta, Curr. Op. Struct. Biol. 2:593-596 (1992)].

Methods for humanizing non-human antibodies are well known in the art. Generally, a humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers [Jones et al., Nature, 321:522-525 (1986); Riechmann et al., Nature, 332:323-327 (1988); Verhoeyen et al., Science, 239:1534-1536 (1988)], by substituting rodent CDRs or CDR sequences for the corresponding sequences of a human antibody. Accordingly, such “humanized” antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

The choice of human variable domains, both light and heavy, to be used in making the humanized antibodies is very important to reduce antigenicity and HAMA response (human anti-mouse antibody) when the antibody is intended for human therapeutic use. According to the so-called “best-fit” method, the sequence of the variable domain of a rodent antibody is screened against the entire library of known human variable domain sequences. The human V domain sequence which is closest to that of the rodent is identified and the human framework region (FR) within it accepted for the humanized antibody (Sims et al., J. Immunol. 151:2296 (1993); Chothia et al., J. Mol. Biol., 196:901 (1987)). Another method uses a particular framework region derived from the consensus sequence of all human antibodies of a particular subgroup of light or heavy chains. The same framework may be used for several different humanized antibodies (Carter et al., Proc. Natl. Acad. Sci. USA, 89:4285 (1992); Presta et al., J. Immunol. 151:2623 (1993)).

It is further important that antibodies be humanized with retention of high binding affinity for the antigen and other favorable biological properties. To achieve this goal, according to a preferred method, humanized antibodies are prepared by a process of analysis of the parental sequences and various conceptual humanized products using three dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the hypervariable region residues are directly and most substantially involved in influencing antigen binding.

Various forms of a humanized anti-TAT antibody are contemplated. For example, the humanized antibody may be an antibody fragment, such as a Fab, which is optionally conjugated with one or more cytotoxic agent(s) in order to generate an immunoconjugate. Alternatively, the humanized antibody may be an intact antibody, such as an intact IgG1 antibody.

As an alternative to humanization, human antibodies can be generated. For example, it is now possible to produce transgenic animals (e.g., mice) that are capable, upon immunization, of producing a full repertoire of human antibodies in the absence of endogenous immunoglobulin production. For example, it has been described that the homozygous deletion of the antibody heavy-chain joining region (J_{H}) gene in chimeric and germ-line mutant mice results in complete inhibition of endogenous antibody production. Transfer of the human germ-line immunoglobulin gene array into such germ-line mutant mice will result in the production of human antibodies upon antigen challenge. See, e.g., Jakobovits et al., Proc. Natl. Acad. Sci. USA, 90:2551 (1993); Jakobovits et al., Nature, 362:255-258 (1993); Bruggemann et al., Year in Immuno. 7:33 (1993); U.S. Pat. Nos. 5,545,806, 5,569,825, 5,591,669 (all of GenPharm); U.S. Pat. No. 5,545,807; and WO 97/17852.

Alternatively, phage display technology (McCafferty et al., Nature 348:552-553 [1990]) can be used to produce human antibodies and antibody fragments in vitro, from immunoglobulin variable (V) domain gene repertoires from unimmunized donors. According to this technique, antibody V domain genes are cloned in-frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B-cell. Phage display can be performed in a variety of formats, reviewed in, e.g., Johnson, Kevin S. and Chiswell, David J., Current Opinion in Structural Biology 3:564-571 (1993). Several sources of V-gene segments can be used for phage display. Clackson et al., Nature, 352:624-628(1991) isolated a diverse array of anti-oxazolone antibodies from a small random combinatorial library of V genes derived from the spleens of immunized mice. A repertoire of V genes from unimmunized human donors can be constructed and antibodies to a diverse array of antigens (including self-antigens) can be isolated essentially following the techniques described by Marks et al., J. Mol. Biol. 222:581-597 (1991), or Griffith et al., EMBO J. 12:725-734 (1993). See, also, U.S. Pat. Nos. 5,565,332 and 5,573,905.

As discussed above, human antibodies may also be generated by in vitro activated B cells (see U.S. Pat. Nos. 5,567,610 and 5,229,275).

4. Antibody Fragments

In certain circumstances there are advantages of using antibody fragments, rather than whole antibodies. The smaller size of the fragments allows for rapid clearance, and may lead to improved access to solid tumors.

Various techniques have been developed for the production of antibody fragments. Traditionally, these fragments were derived via proteolytic digestion of intact antibodies (see, e.g., Morimoto et al., Journal of Biochemical and Biophysical Methods 24:107-117 (1992); and Brennan et al., Science, 229:81 (1985)). However, these fragments can now be produced directly by recombinant host cells. Fab, Fv and ScFv antibody fragments can all be expressed in and secreted from E. coli, thus allowing the facile production of large amounts of these fragments. Antibody fragments can be isolated from the antibody phage libraries discussed above. Alternatively, Fab′-SH fragments can be directly recovered from E. coli and chemically coupled to form F(ab′)₂ fragments (Carter et al., Bio/Technology 10:163-167 (1992)). According to another approach, F(ab′)₂ fragments can be isolated directly from recombinant host cell culture. Fab and F(ab′)₂ fragment with increased in vivo half-life comprising a salvage receptor binding epitope residues are described in U.S. Pat. No. 5,869,046. Other techniques for the production of antibody fragments will be apparent to the skilled practitioner. In other embodiments, the antibody of choice is a single chain Fv fragment (scFv). See WO 93/16185; U.S. Pat. No. 5,571,894; and U.S. Pat. No. 5,587,458. Fv and sFv are the only species with intact combining sites that are devoid of constant regions; thus, they are suitable for reduced nonspecific binding during in vivo use. sFv fusion proteins may be constructed to yield fusion of an effector protein at either the amino or the carboxy terminus of an sFv. See Antibody Engineering, ed. Borrebaeck, supra. The antibody fragment may also be a “linear antibody”, e.g., as described in U.S. Pat. No. 5,641,870 for example. Such linear antibody fragments may be monospecific or bispecific.

5. Bispecific Antibodies

Bispecific antibodies are antibodies that have binding specificities for at least two different epitopes. Exemplary bispecific antibodies may bind to two different epitopes of a TAT protein as described herein. Other such antibodies may combine a TAT binding site with a binding site for another protein. Alternatively, an anti-TAT arm may be combined with an arm which binds to a triggering molecule on a leukocyte such as a T-cell receptor molecule (e.g. CD3), or Fc receptors for IgG (FcγR), such as FcγRI (CD64), FcγRfII (CD32) and FcγRIII (CD16), so as to focus and localize cellular defense mechanisms to the TAT-expressing cell. Bispecific antibodies may also be used to localize cytotoxic agents to cells which express TAT. These antibodies possess a TAT-binding arm and an arm which binds the cytotoxic agent (e.g., saporin, anti-interferon-α, vinca alkaloid, ricin A chain, methotrexate or radioactive isotope hapten). Bispecific antibodies can be prepared as full length antibodies or antibody fragments (e.g., F(ab′)₂ bispecific antibodies).

WO 96/16673 describes a bispecific anti-ErbB2/anti-FcγRIII antibody and U.S. Pat. No. 5,837,234 discloses a bispecific anti-ErbB2/anti-FcγRI antibody. A bispecific anti-ErbB2/Fc α antibody is shown in WO98/02463. U.S. Pat. No. 5,821,337 teaches a bispecific anti-ErbB2/anti-CD3 antibody.

Methods for making bispecific antibodies are known in the art. Traditional production of full length bispecific antibodies is based on the co-expression of two immunoglobulin heavy chain-light chain pairs, where the two chains have different specificities (Millstein et al., Nature 305:537-539 (1983)). Because of the random assortment of immunoglobulin heavy and light chains, these hybridomas (quadromas) produce a potential mixture of 10 different antibody molecules, of which only one has the correct bispecific structure. Purification of the correct molecule, which is usually done by affinity chromatography steps, is rather cumbersome, and the product yields are low. Similar procedures are disclosed in WO 93/08829, and in Traunecker et al., EMBO J. 10:3655-3659 (1991).

According to a different approach, antibody variable domains with the desired binding specificities (antibody-antigen combining sites) are fused to immunoglobulin constant domain sequences. Preferably, the fusion is with an Ig heavy chain constant domain, comprising at least part of the hinge, C_{H}2, and C_{H}3 regions. It is preferred to have the first heavy-chain constant region (C_{H}1) containing the site necessary for light chain bonding, present in at least one of the fusions. DNAs encoding the immunoglobulin heavy chain fusions and, if desired, the immunoglobulin light chain, are inserted into separate expression vectors, and are co-transfected into a suitable host cell. This provides for greater flexibility in adjusting the mutual proportions of the three polypeptide fragments in embodiments when unequal ratios of the three polypeptide chains used in the construction provide the optimum yield of the desired bispecific antibody. It is, however, possible to insert the coding sequences for two or all three polypeptide chains into a single expression vector when the expression of at least two polypeptide chains in equal ratios results in high yields or when the ratios have no significant affect on the yield of the desired chain combination.

In a preferred embodiment of this approach, the bispecific antibodies are composed of a hybrid immunoglobulin heavy chain with a first binding specificity in one arm, and a hybrid immunoglobulin heavy chain-light chain pair (providing a second binding specificity) in the other arm. It was found that this asymmetric structure facilitates the separation of the desired bispecific compound from unwanted immunoglobulin chain combinations, as the presence of an immunoglobulin light chain in only one half of the bispecific molecule provides for a facile way of separation. This approach is disclosed in WO 94/04690. For further details of generating bispecific antibodies see, for example, Suresh et al., Methods in Enzymology 121:210 (1986).

According to another approach described in U.S. Pat. No. 5,731,168, the interface between a pair of antibody molecules can be engineered to maximize the percentage of heterodimers which are recovered from recombinant cell culture. The preferred interface comprises at least a part of the C_{H}3 domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers.

Bispecific antibodies include cross-linked or “heteroconjugate” antibodies. For example, one of the antibodies in the heteroconjugate can be coupled to avidin, the other to biotin. Such antibodies have, for example, been proposed to target immune system cells to unwanted cells (U.S. Pat. No. 4,676,980), and for treatment of HIV infection (WO 91/00360, WO 92/200373, and EP 03089). Heteroconjugate antibodies may be made using any convenient cross-linking methods. Suitable cross-liting agents are well known in the art, and are disclosed in U.S. Pat. No. 4,676,980, along with a number of cross-linking techniques.

Techniques for generating bispecific antibodies from antibody fragments have also been described in the literature. For example, bispecific antibodies can be prepared using chemical linkage. Brennan et al., Science 229:81 (1985) describe a procedure wherein intact antibodies are proteolytically cleaved to generate F(ab′)₂ fragments. These fragments are reduced in the presence of the dithiol complexing agent, sodium arsenite, to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Recent progress has facilitated the direct recovery of Fab′-SH fragments from E. coli, which can be chemically coupled to form bispecific antibodies. Shalaby et al., J. Exp. Med. 175: 217-225 (1992) describe the production of a fully humanized bispecific antibody F(ab′)₂ molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various techniques for making and isolating bispecific antibody fragments directly from recombinant cell culture have also been described. For example, bispecific antibodies have been produced using leucine zippers. Kostelny et al., J. Immunol. 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA 90:6444-6448 (1993) has provided an alternative mechanism for making bispecific antibody fragments. The fragments comprise a V_{H} connected to a V_{L} by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See Gruber et al., J. Immunol., 152:5368 (1994).

Antibodies with more than two valencies are contemplated. For example, trispecific antibodies can be prepared. Tutt et al., J. Immunol. 147:60 (1991).

6. Heteroconjugate Antibodies

Heteroconjugate antibodies are also within the scope of the present invention. Heteroconjugate antibodies are composed of two covalently joined antibodies. Such antibodies have, for example, been proposed to target immune system cells to unwanted cells [U.S. Pat. No. 4,676,980], and for treatment of HIV infection [WO 91/00360; WO 92/200373; EP 03089]. It is contemplated that the antibodies may be prepared in vitro using known methods in synthetic protein chemistry, including those involving crosslinking agents. For example, immunotoxins may be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980.

7. Multivalent Antibodies

A multivalent antibody may be internalized (and/or catabolized) faster than a bivalent antibody by a cell expressing an antigen to which the antibodies bind. The antibodies of the present invention can be multivalent antibodies (which are other than of the IgM class) with three or more antigen binding sites (e.g. tetravalent antibodies), which can be readily produced by recombinant expression of nucleic acid encoding the polypeptide chains of the antibody. The multivalent antibody can comprise a dimerization domain and three or more antigen binding sites. The preferred dimerization domain comprises (or consists of) an Fc region or a hinge region. In this scenario, the antibody will comprise an Fc region and three or more antigen binding sites amino-terminal to the Fc region. The preferred multivalent antibody herein comprises (or consists of) three to about eight, but preferably four, antigen binding sites. The multivalent antibody comprises at least one polypeptide chain (and preferably two polypeptide chains), wherein the polypeptide chain(s) comprise two or more variable domains. For instance, the polypeptide chain(s) may comprise VD1-(X1)ₙ-VD2-(X2)ₙ-Fc, wherein VD1 is a first variable domain, VD2 is a second variable domain, Fc is one polypeptide chain of an Fc region, X1 and X2 represent an amino acid or polypeptide, and n is 0 or 1. For instance, the polypeptide chain(s) may comprise: VH-CH1-flexible linker-VH-CH1-Fc region chain; or VH-CH1-VH-CH1-Fc region chain. The multivalent antibody herein preferably further comprises at least two (and preferably four) light chain variable domain polypeptides. The multivalent antibody herein may, for instance, comprise from about two to about eight light chain variable domain polypeptides. The light chain variable domain polypeptides contemplated here comprise a light chain variable domain and, optionally, further comprise a CL domain.

8. Effector Function Engineering

It may be desirable to modify the antibody of the invention with respect to effector function, e.g., so as to enhance antigen-dependent cell-mediated cyotoxicity (ADCC) and/or complement dependent cytotoxicity (CDC) of the antibody. This may be achieved by introducing one or more amino acid substitutions in an Fc region of the antibody. Alternatively or additionally, cysteine residue(s) may be introduced in the Fc region, thereby allowing interchain disulfide bond formation in this region. The homodimeric antibody thus generated may have improved internalization capability and/or increased complement-mediated cell killing and antibody-dependent cellular cytotoxicity (ADCC). See Caron et al., J. Exp Med. 176:1191-1195 (1992) and Shopes, B. J. Immunol. 148:2918-2922 (1992). Homodimeric antibodies with enhanced anti-tumor activity may also be prepared using heterobifunctional cross-linkers as described in Wolff et al., Cancer Research 53:2560-2565 (1993). Alternatively, an antibody can be engineered which has dual Fc regions and may thereby have enhanced complement lysis and ADCC capabilities. See Stevenson et al., Anti-Cancer Drug Design 3:219-230 (1989).

To increase the serum half life of the antibody, one may incorporate a salvage receptor binding epitope into the antibody (especially an antibody fragment) as described in U.S. Pat. No. 5,739,277, for example. As used herein, the term “salvage receptor binding epitope” refers to an epitope of the Pc region of an IgG molecule (e.g., IgG₁, IgG₂, IgG₃, or IgG₄) that is responsible for increasing the in vivo serum half-life of the IgG molecule.

9. Immunoconjugates

The invention also pertains to immunoconjugates comprising an antibody conjugated to a cytotoxic agent such as a chemotherapeutic agent, a growth inhibitory agent, a toxin (e.g., an enzymatically active toxin of bacterial, fungal, plant, or animal origin, or fragments thereof), or a radioactive isotope (i.e., a radioconjugate).

Chemotherapeutic agents useful in the generation of such immunoconjugates have been described above. Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes. A variety of radionuclides are available for the production of radioconjugated antibodies. Examples include ²¹²Bi, ¹³¹I, ¹³¹In, ⁹⁰Y, and ¹⁸⁶Re. Conjugates of the antibody and cytotoxic agent are made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al., Science, 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026.

Conjugates of an antibody and one or more small molecule toxins, such as a calicheamicin, maytansinoids, a trichothene, and CC1065, and the derivatives of these toxins that have toxin activity, are also contemplated herein.

Maytansine and Maytansinoids

In one preferred embodiment, an anti-TAT antibody (full length or fragments) of the invention is conjugated to one or more maytansinoid molecules.

Maytansinoids are mitototic inhibitors which act by inhibiting tubulin polymerization. Maytansine was first isolated from the east African shrub Maytenus serrata (U.S. Pat. No. 3,896,111). Subsequently, it was discovered that certain microbes also produce maytansinoids, such as maytansinol and C-3 maytansinol esters (U.S. Pat. No. 4,151,042). Synthetic maytansinol and derivatives and analogues thereof are disclosed, for example, in U.S. Pat. Nos. 4,137,230; 4,248,870; 4,256,746; 4,260,608; 4,265,814; 4,294,757; 4,307,016; 4,308,268; 4,308,269; 4,309,428; 4,313,946; 4,315,929; 4,317,821; 4,322,348; 4,331,598; 4,361,650; 4,364,866; 4,424,219; 4,450,254; 4,362,663; and 4,371,533, the disclosures of which are hereby expressly incorporated by reference.

Maytansinoid-Antibody Conjugates

In an attempt to improve their therapeutic index, maytansine and maytansinoids have been conjugated to antibodies specifically binding to tumor cell antigens. Immunoconjugates containing maytansinoids and their therapeutic use are disclosed, for example, in U.S. Pat. Nos. 5,208,020, 5,416,064 and European Patent EP 0 425 235 B1, the disclosures of which are hereby expressly incorporated by reference. Liu et al., Proc. Natl. Acad. Sci. USA 93:8618-8623 (1996) described immunoconjugates comprising a maytansinoid designated DM1 linked to the monoclonal antibody C242 directed against human colorectal cancer. The conjugate was found to be highly cytotoxic towards cultured colon cancer cells, and showed antitumor activity in an in vivo tumor growth assay. Chari et al., Cancer Research 52:127-131 (1992) describe immunoconjugates in which a maytansinoid was conjugated via a disulfide linker to the murine antibody A7 binding to an antigen on human colon cancer cell lines, or to another murine monoclonal antibody TA.1 that binds the HER-2/neu oncogene. The cytotoxicity of the TA.1-maytansonoid conjugate was tested in vitro on the human breast cancer cell line SK-BR-3, which expresses 3×10⁵ HER-2 surface antigens per cell. The drug conjugate achieved a degree of cytotoxicity similar to the free maytansonid drug, which could be increased by increasing the number of maytansinoid molecules per antibody molecule. The A7-maytansinoid conjugate showed low systemic cytotoxicity in mice.

Anti-TAT Polypeptide Antibody-Maytansinoid Conjugates (Immunoconjugates)

Anti-TAT antibody-maytansinoid conjugates are prepared by chemically linking an anti-TAT antibody to a maytansinoid molecule without significantly diminishing the biological activity of either the antibody or the maytansinoid molecule. An average of 3-4 maytansinoid molecules conjugated per antibody molecule has shown efficacy in enhancing cytotoxicity of target cells without negatively affecting the function or solubility of the antibody, although even one molecule of toxin/antibody would be expected to enhance cytotoxicity over the use of naked antibody. Maytansinoids are well known in the art and can be synthesized by known techniques or isolated from natural sources. Suitable maytansinoids are disclosed, for example, in U.S. Pat. No. 5,208,020 and in the other patents and nonpatent publications referred to hereinabove. Preferred maytansinoids are maytansinol and maytansinol analogues modified in the aromatic ring or at other positions of the maytansinol molecule, such as various maytansinol esters.

There are many linking groups known in the art for making antibody-maytansinoid conjugates, including, for example, those disclosed in U.S. Pat. No. 5,208,020 or EP Patent 0 425 235 B1, and Chari et al., Cancer Research 52:127-131 (1992). The linking groups include disufide groups, thioether groups, acid labile groups, photolabile groups, peptidase labile groups, or esterase labile groups, as disclosed in the above-identified patents, disulfide and thioether groups being preferred.

Conjugates of the antibody and maytansinoid may be made using a variety of bifunctional protein coupling agents such as N-succinimidyl-3-(2-pyridyldithio) propionate (SPDP), succinimidyl-4-(N-maleimidomethyl) cyclohexane-1-carboxylate, iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(pdiazoniumbenzoyl)-ethylenedianine), diisocyanates (such as toluene 2,6-diisocyanate), and bis-active fluorine compounds (such as 1,5-difluoro-2,4-dinitrobenzene). Particularly preferred coupling agents include N-succinimidyl-3-(2-pyridyldithio) propionate (SPDP) (Carlsson et al., Biochem. J. 173:723-737 [1978]) and N-succinimidyl-4-(2-pyridylthio)pentanoate (SPP) to provide for a disulfide linkage.

The linker may be attached to the maytansinoid molecule at various positions, depending on the type of the link. For example, an ester linkage may be formed by reaction with a hydroxyl group using conventional coupling techniques. The reaction may occur at the C-3 position having a hydroxyl group, the C-14 position modified with hyrdoxymethyl, the C-15 position modified with a hydroxyl group, and the C-20 position having a hydroxyl group. In a preferred embodiment, the linkage is formed at the C-3 position of maytansinol or a maytansinol analogue.

Calicheamicin

Another immunoconjugate of interest comprises an anti-TAT antibody conjugated to one or more calicheamicin molecules. The calicheamicin family of antibiotics are capable of producing double-stranded DNA breaks at sub-picomolar concentrations. For the preparation of conjugates of the calicheamicin family, see U.S. Pat. Nos. 5,712,374, 5,714,586, 5,739,116, 5,767,285, 5,770,701, 5,770,710, 5,773,001, 5,877,296 (all to American Cyanamid Company). Structural analogues of calicheamicin which may be used include, but are not limited to, γ₁^{I}, α₂^{I}, α₃^{I}, N-acetyl-γ₁^{I}, PSAG and θ₁^{I} (Hinman et al., Cancer Research 53:3336-3342 (1993), Lode et al., Cancer Research 58:2925-2928 (1998) and the aforementioned U.S. patents to American Cyanamid). Another anti-tumor drug that the antibody can be conjugated is QFA which is an antifolate. Both calicheamicin and QFA have intracellular sites of action and do not readily cross the plasma membrane. Therefore, cellular uptake of these agents through antibody mediated internalization greatly enhances their cytotoxic effects.

Other Cytotoxic Agents

Other antitumor agents that can be conjugated to the anti-TAT antibodies of the invention include BCNU, streptozoicin, vincristine and 5-fluorouracil, the family of agents known collectively LL-E33288 complex described in U.S. Pat. Nos. 5,053,394, 5,770,710, as well as esperamicins (U.S. Pat. No. 5,877,296).

Enzymatically active toxins and fragments thereof which can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin and the tricothecenes. See, for example, WO 93/21232 published Oct. 28, 1993.

The present invention further contemplates an immunoconjugate formed between an antibody and a compound with nucleolytic activity (e.g., a ribonuclease or a DNA endonuclease such as a deoxyribonuclease; DNase).

For selective destruction of the tumor, the antibody may comprise a highly radioactive atom. A variety of radioactive isotopes are available for the production of radioconjugated anti-TAT antibodies. Examples include At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu. When the conjugate is used for diagnosis, it may comprise a radioactive atom for scintigraphic studies, for example tc^{99m} or I¹²³, or a spin label for nuclear magnetic resonance (NMR) imaging (also known as magnetic resonance imaging, mri), such as iodine-123 again, iodine-131, indium-111, fluorine-19, carbon-13, nitrogen-15, oxygen-17, gadolinium, manganese or iron.

The radio- or other labels may be incorporated in the conjugate in known ways. For example, the peptide may be biosynthesized or may be synthesized by chemical amino acid synthesis using suitable amino acid precursors involving, for example, fluorine-19 in place of hydrogen. Labels such as tc^{99m} or I¹²³, .Re¹⁸⁶, Re¹⁸⁸ and In¹¹¹ can be attached via a cysteine residue in the peptide. Yttrium-90 can be attached via a lysine residue. The IODOGEN method (Fraker et al (1978) Biochem. Biophys. Res. Commun. 80: 49-57 can be used to incorporate iodine-123. “Monoclonal Antibodies Immunoscintigraphy” (Chatal, CRC Press 1989) describes other methods in detail.

Conjugates of the antibody and cytotoxic agent may be made using a variety of bifunctional protein coupling agents such as N-succinimidyl-3-(2-pyridyldithio) propionate (SPDP), succinimidyl-4-(N-maleimidomethyl) cyclohexane-1-carboxylate, iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al., Science 238:1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026. The linker may be a “cleavable linker” facilitating release of the cytotoxic drug in the cell. For example, an acid-labile linker, peptidase-sensitive linker, photolabile linker, dimethyl linker or disulfide-containing linker (Chari et al., Cancer Research 52:127-131 (1992); U.S. Pat. No. 5,208,020) may be used.

Alternatively, a fusion protein comprising the anti-TAT antibody and cytotoxic agent may be made, e.g., by recombinant techniques or peptide synthesis. The length of DNA may comprise respective regions encoding the two portions of the conjugate either adjacent one another or separated by a region encoding a linker peptide which does not destroy the desired properties of the conjugate.

In yet another embodiment, the antibody may be conjugated to a “receptor” (such streptavidin) for utilization in tumor pre-targeting wherein the antibody-receptor conjugate is administered to the patient, followed by removal of unbound conjugate from the circulation using a clearing agent and then administration of a “ligand” (e.g., avidin) which is conjugated to a cytotoxic agent (e.g., a radionucleotide).

10. Immunoliposomes

The anti-TAT antibodies disclosed herein may also be formulated as immunoliposomes. A “liposome” is a small vesicle composed of various types of lipids, phospholipids and/or surfactant which is useful for delivery of a drug to a mammal. The components of the liposome are commonly arranged in a bilayer formation, similar to the lipid arrangement of biological membranes. Liposomes containing the antibody are prepared by methods known in the art, such as described in Epstein et al., Proc. Natl. Acad. Sci. USA 82:3688 (1985); Hwang et al., Proc. Natl. Acad. Sci. USA 77:4030 (1980); U.S. Pat. Nos. 4,485,045 and 4,544,545; and WO97/38731 published Oct. 23, 1997. Liposomes with enhanced circulation time are disclosed in U.S. Pat. No. 5,013,556.

Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter. Fab′ fragments of the antibody of the present invention can be conjugated to the liposomes as described in Martin et al., J. Biol. Chem. 257:286-288 (1982) via a disulfide interchange reaction. A chemotherapeutic agent is optionally contained within the liposome. See Gabizon et al., J. National Cancer Inst. 81(19):1484 (1989).

B. TAT Binding Oligopeptides

TAT binding oligopeptides of the present invention are oligopeptides that bind, preferably specifically, to a TAT polypeptide as described herein. TAT binding oligopeptides may be chemically synthesized using known oligopeptide synthesis methodology or may be prepared and purified using recombinant technology. TAT binding oligopeptides are usually at least about 5 amino acids in length, alternatively at least about 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 143, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 amino acids in length or more, wherein such oligopeptides that are capable of binding, preferably specifically, to a TAT polypeptide as described herein. TAT binding oligopeptides may be identified without undue experimentation using well known techniques. In this regard, it is noted that techniques for screening oligopeptide libraries for oligopeptides that are capable of specifically binding to a polypeptide target are well known in the art (see, e.g., U.S. Pat. Nos. 5,556,762, 5,750,373, 4,708,871, 4,833,092, 5,223,409, 5,403,484, 5,571,689, 5,663,143; PCT Publication Nos. WO 84/03506 and WO84/03564; Geysen et al., Proc. Natl. Acad. Sci. U.S.A., 81:3998-4002 (1984); Geysen et al., Proc. Natl. Acad. Sci. U.S.A., 82:178-182 (1985); Geysen et al., in Synthetic Peptides as Antigens, 130-149 (1986); Geysen et al., J. Immunol. Meth., 102:259-274 (1987); Schoofs et al., J. Immunol., 140:611-616 (1988), Cwirla, S. E. et al. (1990) Proc. Natl. Acad. Sci. USA, 87:6378; Lowman, H. B. et al. (1991) Biochemistry, 30:10832; Clackson, T. et al. (1991) Nature, 352: 624; Marks, J. D. et al. (1991), J. Mol. Biol., 222:581; Kang, A. S. et al. (1991) Proc. Natl. Acad. Sci. USA, 88:8363, and Smith, G. P. (1991) Current Opin. Biotechnol., 2:668).

In this regard, bacteriophage (phage) display is one well known technique which allows one to screen large oligopeptide libraries to identify member(s) of those libraries which are capable of specifically binding to a polypeptide target. Phage display is a technique by which variant polypeptides are displayed as fusion proteins to the coat protein on the surface of bacteriophage particles (Scott, J. K. and Smith, G. P. (1990) Science 249: 386). The utility of phage display lies in the fact that large libraries of selectively randomized protein variants (or randomly cloned cDNAs) can be rapidly and efficiently sorted for those sequences that bind to a target molecule with high affinity. Display of peptide (Cwirla, S. E. et al. (1990) Proc. Natl. Acad. Sci. USA, 87:6378) or protein (Lowman, H. B. et al. (1991) Biochemistry, 30:10832; Clackson, T. et al. (1991) Nature, 352: 624; Marks, J. D. et al. (1991), J. Mol. Biol., 222:581; Kang, A. S. et al. (1991) Proc. Natl. Acad. Sci. USA, 88:8363) libraries on phage have been used for screening millions of polypeptides or oligopeptides for ones with specific binding properties (Smith, G. P. (1991) Current Opin. Biotechnol., 2:668). Sorting phage libraries of random mutants requires a strategy for constructing and propagating a large number of variants, a procedure for affinity purification using the target receptor, and a means of evaluating the results of binding enrichments. U.S. Pat. Nos. 5,223,409, 5,403,484, 5,571,689, and 5,663,143.

Although most phage display methods have used filamentous phage, lambdoid phage display systems (WO 95/34683; U.S. Pat. No. 5,627,024), T4 phage display systems (Ren, Z-J. et al. (1998) Gene 215:439; Zhu, Z. (1997) CAN 33:534; Jiang, J. et al. (1997) can 128:44380; Ren, Z-J. et al. (1997) CAN 127:215644; Ren, Z-J. (1996) Protein Sci. 5:1833; Efimov, V. P. et al. (1995) Virus Genes 10:173) and T7 phage display systems (Smith, G. P. and Scott, J. K. (1993) Methods in Enzymology, 217, 228-257; U.S. Pat. No. 5,766,905) are also known.

Many other improvements and variations of the basic phage display concept have now been developed. These improvements enhance the ability of display systems to screen peptide libraries for binding to selected target molecules and to display functional proteins with the potential of screening these proteins for desired properties. Combinatorial reaction devices for phage display reactions have been developed (WO 98/14277) and phage display libraries have been used to analyze and control bimolecular interactions (WO 98/20169; WO 98/20159) and properties of constrained helical peptides (WO 98/20036). WO 97/35196 describes a method of isolating an affinity ligand in which a phage display library is contacted with one solution in which the ligand will bind to a target molecule and a second solution in which the affinity ligand will not bind to the target molecule, to selectively isolate binding ligands. WO 97/46251 describes a method of biopanning a random phage display library with an affinity purified antibody and then isolating binding phage, followed by a micropanning process using microplate wells to isolate high affinity binding phage. The use of Staphlylococcus aureus protein A as an affinity tag has also been reported (Li et al. (1998) Mol Biotech., 9:187). WO 97/47314 describes the use of substrate subtraction libraries to distinguish enzyme specificities using a combinatorial library which may be a phage display library. A method for selecting enzymes suitable for use in detergents using phage display is described in WO 97/09446. Additional methods of selecting specific binding proteins are described in U.S. Pat. Nos. 5,498,538, 5,432,018, and WO 98/15833.

Methods of generating peptide libraries and screening these libraries are also disclosed in U.S. Pat. Nos. 5,723,286, 5,432,018, 5,580,717, 5,427,908, 5,498,530, 5,770,434, 5,734,018, 5,698,426, 5,763,192, and 5,723,323.

C. TAT Binding Organic Molecules

TAT binding organic molecules are organic molecules other than oligopeptides or antibodies as defined herein that bind, preferably specifically, to a TAT polypeptide as described herein. TAT binding organic molecules may be identified and chemically synthesized using known methodology (see, e.g., PCT Publication Nos. WO00/00823 and WO00/39585). TAT binding organic molecules are usually less than about 2000 daltons in size, alternatively less than about 1500, 750, 500, 250 or 200 daltons in size, wherein such organic molecules that are capable of binding, preferably specifically, to a TAT polypeptide as described herein may be identified without undue experimentation using well known techniques. In this regard, it is noted that techniques for screening organic molecule libraries for molecules that are capable of binding to a polypeptide target are well known in the art (see, e.g., PCT Publication Nos. WO00/00823 and WO00/39585). TAT binding organic molecules may be, for example, aldehydes, ketones, oximes, hydrazones, semicarbazones, carbazides, primary amines, secondary amines, tertiary amines, N-substituted hydrazines, hydrazides, alcohols, ethers, thiols, thioethers, disulfides, carboxylic acids, esters, amides, ureas, carbamates, carbonates, ketals, thioketals, acetals, thioacetals, aryl halides, aryl sulfonates, alkyl halides, alkyl sulfonates, aromatic compounds, heterocyclic compounds, anilines, alkenes, alkynes, diols, amino alcohols, oxazolidines, oxazolines, thiazolidines, thiazolines, enamines, sulfonamides, epoxides, aziridines, isocyanates, sulfonyl chlorides, diazo compounds, acid chlorides, or the like.

D. Screening for Anti-TAT Antibodies, TAT Binding Oligopeptides and TAT Binding Organic Molecules with the Desired Properties

Techniques for generating antibodies, oligopeptides and organic molecules that bind to TAT polypeptides have been described above. One may further select antibodies, oligopeptides or other organic molecules with certain biological characteristics, as desired.

The growth inhibitory effects of an anti-TAT antibody, oligopeptide or other organic molecule of the invention may be assessed by methods known in the art, e.g., using cells which express a TAT polypeptide either endogenously or following transfection with the TAT gene. For example, appropriate tumor cell lines and TAT-transfected cells may treated with an anti-TAT monoclonal antibody, oligopeptide or other organic molecule of the invention at various concentrations for a few days (e.g., 2-7) days and stained with crystal violet or MTT or analyzed by some other colorimetric assay. Another method of measuring proliferation would be by comparing ³H-thymidine uptake by the cells treated in the presence or absence an anti-TAT antibody, TAT binding oligopeptide or TAT binding organic molecule of the invention. After treatment, the cells are harvested and the amount of radioactivity incorporated into the DNA quantitated in a scintillation counter. Appropriate positive controls include treatment of a selected cell line with a growth inhibitory antibody known to inhibit growth of that cell line. Growth inhibition of tumor cells in vivo can be determined in various ways known in the art. Preferably, the tumor cell is one that overexpresses a TAT polypeptide. Preferably, the anti-TAT antibody, TAT binding oligopeptide or TAT binding organic molecule will inhibit cell proliferation of a TAT-expressing tumor cell in vitro or in vivo by about 25-100% compared to the untreated tumor cell, more preferably, by about 30-100%, and even more preferably by about 50-100% or 70-100%, in one embodiment, at an antibody concentration of about 0.5 to 30 μg/ml. Growth inhibition can be measured at an antibody concentration of about 0.5 to 30 μg/ml or about 0.5 nM to 200 nM in cell culture, where the growth inhibition is determined 1-10 days after exposure of the tumor cells to the antibody. The antibody is growth inhibitory in vivo if administration of the anti-TAT antibody at about 1 μg/kg to about 100 mg/kg body weight results in reduction in tumor size or reduction of tumor cell proliferation within about 5 days to 3 months from the first administration of the antibody, preferably within about 5 to 30 days.

To select for an anti-TAT antibody, TAT binding oligopeptide or TAT binding organic molecule which induces cell death, loss of membrane integrity as indicated by, e.g., propidium iodide (PI), trypan blue or 7AAD uptake may be assessed relative to control. A PI uptake assay can be performed in the absence of complement and immune effector cells. TAT polypeptide-expressing tumor cells are incubated with medium alone or medium containing the appropriate anti-TAT antibody (e.g, at about 10 μg/ml), TAT binding oligopeptide or TAT binding organic molecule. The cells are incubated for a 3 day time period. Following each treatment, cells are washed and aliquoted into 35 mm strainer-capped 12×75 tubes (1 ml per tube, 3 tubes per treatment group) for removal of cell clumps. Tubes then receive PI (10 μg/ml). Samples may be analyzed using a FACSCAN® flow cytometer and FACSCONVERT® CellQuest software (Becton Dickinson). Those anti-TAT antibodies, TAT binding oligopeptides or TAT binding organic molecules that induce statistically significant levels of cell death as determined by PI uptake may be selected as cell death-inducing anti-TAT antibodies, TAT binding oligopeptides or TAT binding organic molecules.

To screen for antibodies, oligopeptides or other organic molecules which bind to an epitope on a TAT polypeptide bound by an antibody of interest, a routine cross-blocking assay such as that described in Antibodies, A Laboratory Manual, Cold Spring Harbor Laboratory, Ed Harlow and David Lane (1988), can be performed. This assay can be used to determine if a test antibody, oligopeptide or other organic molecule binds the same site or epitope as a known anti-TAT antibody. Alternatively, or additionally, epitope mapping can be performed by methods known in the art. For example, the antibody sequence can be mutagenized such as by alanine scanning, to identify contact residues. The mutant antibody is initailly tested for binding with polyclonal antibody to ensure proper folding. In a different method, peptides corresponding to different regions of a TAT polypeptide can be used in competition assays with the test antibodies or with a test antibody and an antibody with a characterized or known epitope.

E. Antibody Dependent Enzyme Mediated Prodrug Therapy (ADEPT)

The antibodies of the present invention may also be used in ADEPT by conjugating the antibody to a prodrug-activating enzyme which converts a prodrug (e.g., a peptidyl chemotherapeutic agent, see WO81/01145) to an active anti-cancer drug. See, for example, WO 88/07378 and U.S. Pat. No. 4,975,278.

The enzyme component of the immunoconjugate useful for ADEPT includes any enzyme capable of acting on a prodrug in such a way so as to covert it into its more active, cytotoxic form.

Enzymes that are useful in the method of this invention include, but are not limited to, alkaline phosphatase useful for converting phosphate-containing prodrugs into free drugs; arylsulfatase useful for converting sulfate-containing prodrugs into free drugs; cytosine deaminase useful for converting non-toxic 5-fluorocytosine into the anti-cancer drug, 5-fluorouracil; proteases, such as serratia protease, thermolysin, subtilisin, carboxypeptidases and cathepsins (such as cathepsins B and L), that are useful for converting peptide-containing prodrugs into free drugs; D-alanylcarboxypeptidases, useful for converting prodrugs that contain D-amino acid substituents; carbohydrate-cleaving enzymes such as β-galactosidase and neuraminidase useful for converting glycosylated prodrugs into free drugs; β-lactamase useful for converting drugs derivatized with β-lactams into free drugs; and penicillin amidases, such as penicillin V amidase or penicillin G amidase, useful for converting drugs derivatized at their amine nitrogens with phenoxyacetyl or phenylacetyl groups, respectively, into free drugs. Alternatively, antibodies with enzymatic activity, also known in the art as “abzymes”, can be used to convert the prodrugs of the invention into free active drugs (see, e.g., Massey, Nature 328:457-458 (1987)). Antibody-abzyme conjugates can be prepared as described herein for delivery of the abzyme to a tumor cell population.

The enzymes of this invention can be covalently bound to the anti-TAT antibodies by techniques well known in the art such as the use of the heterobifunctional crosslinking reagents discussed above. Alternatively, fusion proteins comprising at least the antigen binding region of an antibody of the invention linked to at least a functionally active portion of an enzyme of the invention can be constructed using recombinant DNA techniques well known in the art (see, e.g., Neuberger et al., Nature 312:604-608 (1984).

F. Full-Length TAT Polypeptides

The present invention also provides newly identified and isolated nucleotide sequences encoding polypeptides referred to in the present application as TAT polypeptides. In particular, cDNAs (partial and full-length) encoding various TAT polypeptides have been identified and isolated, as disclosed in further detail in the Examples below.

As disclosed in the Examples below, various cDNA clones have been deposited with the ATCC. The actual nucleotide sequences of those clones can readily be determined by the skilled artisan by sequencing of the deposited clone using routine methods in the art. The predicted amino acid sequence can be determined from the nucleotide sequence using routine skill. For the TAT polypeptides and encoding nucleic acids described herein, in some cases, Applicants have identified what is believed to be the reading frame best identifiable with the sequence information available at the time.

G. Anti-TAT Antibody and TAT Polypeptide Variants

In addition to the anti-TAT antibodies and full-length native sequence TAT polypeptides described herein, it is contemplated that anti-TAT antibody and TAT polypeptide variants can be prepared. Anti-TAT antibody and TAT polypeptide variants can be prepared by introducing appropriate nucleotide changes into the encoding DNA, and/or by synthesis of the desired antibody or polypeptide. Those skilled in the art will appreciate that amino acid changes may alter post-translational processes of the anti-TAT antibody or TAT polypeptide, such as changing the number or position of glycosylation sites or altering the membrane anchoring characteristics.

Variations in the anti-TAT antibodies and TAT polypeptides described herein, can be made, for example, using any of the techniques and guidelines for conservative and non-conservative mutations set forth, for instance, in U.S. Pat. No. 5,364,934. Variations may be a substitution, deletion or insertion of one or more codons encoding the antibody or polypeptide that results in a change in the amino acid sequence as compared with the native sequence antibody or polypeptide. Optionally the variation is by substitution of at least one amino acid with any other amino acid in one or more of the domains of the anti-TAT antibody or TAT polypeptide. Guidance in determining which amino acid residue may be inserted, substituted or deleted without adversely affecting the desired activity may be found by comparing the sequence of the anti-TAT antibody or TAT polypeptide with that of homologous known protein molecules and minimizing the number of amino acid sequence changes made in regions of high homology. Amino acid substitutions can be the result of replacing one amino acid with another amino acid having similar structural and/or chemical properties, such as the replacement of a leucine with a serine, i.e., conservative amino acid replacements. Insertions or deletions may optionally be in the range of about 1 to 5 amino acids. The variation allowed may be determined by systematically making insertions, deletions or substitutions of amino acids in the sequence and testing the resulting variants for activity exhibited by the full-length or mature native sequence.

Anti-TAT antibody and TAT polypeptide fragments are provided herein. Such fragments may be truncated at the N-terminus or C-terminus, or may lack internal residues, for example, when compared with a full length native antibody or protein. Certain fragments lack amino acid residues that are not essential for a desired biological activity of the anti-TAT antibody or TAT polypeptide.

Anti-TAT antibody and TAT polypeptide fragments may be prepared by any of a number of conventional techniques. Desired peptide fragments may be chemically synthesized. An alternative approach involves generating antibody or polypeptide fragments by enzymatic digestion, e.g., by treating the protein with an enzyme known to cleave proteins at sites defined by particular amino acid residues, or by digesting the DNA with suitable restriction enzymes and isolating the desired fragment. Yet another suitable technique involves isolating and amplifying a DNA fragment encoding a desired antibody or polypeptide fragment, by polymerase chain reaction (PCR). Oligonucleotides that define the desired termini of the DNA fragment are employed at the 5′ and 3′ primers in the PCR. Preferably, anti-TAT antibody and TAT polypeptide fragments share at least one biological and/or immunological activity with the native anti-TAT antibody or TAT polypeptide disclosed herein.

In particular embodiments, conservative substitutions of interest are shown in Table 6 under the heading of preferred substitutions. If such substitutions result in a change in biological activity, then more substantial changes, denominated exemplary substitutions in Table 6, or as further described below in reference to amino acid classes, are introduced and the products screened.

TABLE 6

| Original | Exemplary | Preferred |
|---|---|---|
| Residue | Substitutions | Substitutions |
| Ala (A) | val; leu; ile | val |
| Arg (R) | lys; gln; asn | lys |
| Asn (N) | gln; his; lys; arg | gln |
| Asp (D) | glu | glu |
| Cys (C) | ser | ser |
| Gln (Q) | asn | asn |
| Glu (E) | asp | asp |
| Gly (G) | pro; ala | ala |
| His (H) | asn; gln; lys; arg | arg |
| Ile (I) | leu; val; met; ala; phe; | leu |
| | norleucine | |
| Leu (L) | norleucine; ile; val; | ile |
| | met; ala; phe | |
| Lys (K) | arg; gln; asn | arg |
| Met (M) | leu; phe; ile | leu |
| Phe (F) | leu; val; ile; ala; tyr | leu |
| Pro (P) | ala | ala |
| Ser (S) | thr | thr |
| Thr (T) | ser | ser |
| Trp (W) | tyr; phe | tyr |
| Tyr (Y) | trp; phe; thr; ser | phe |
| Val (V) | ile; leu; met; phe; | leu |
| | ala; norleucine | |

Substantial modifications in function or immunological identity of the anti-TAT antibody or TAT polypeptide are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring residues are divided into groups based on common side-chain properties:

(1) hydrophobic: norleucine, met, ala, val, leu, ile;

(2) neutral hydrophilic: cys, ser, thr;

(3) acidic: asp, glu;

(4) basic: asn, gln, his, lys, arg;

(5) residues that influence chain orientation: gly, pro; and

(6) aromatic: trp, tyr, phe.

Non-conservative substitutions will entail exchanging a member of one of these classes for another class. Such substituted residues also may be introduced into the conservative substitution sites or, more preferably, into the remaining (non-conserved) sites.

The variations can be made using methods known in the art such as oligonucleotide-mediated (site-directed) mutagenesis, alanine scanning, and PCR mutagenesis. Site-directed mutagenesis [Carter et al. Nucl. Acids Res., 13:4331 (1986); Zoller et al., Nucl. Acids Res., 10:6487 (1987)], cassette mutagenesis [Wells et al., Gene, 34:315 (1985)], restriction selection mutagenesis [Wells et al., Philos. Trans. R. Soc. London SerA, 317:415 (1986)] or other known techniques can be performed on the cloned DNA to produce the anti-TAT antibody or TAT polypeptide variant DNA.

Scanning amino acid analysis can also be employed to identify one or more amino acids along a contiguous sequence. Among the preferred scanning amino acids are relatively small, neutral amino acids. Such amino acids include alanine, glycine, serine, and cysteine. Alanine is typically a preferred scanning amino acid among this group because it eliminates the side-chain beyond the beta-carbon and is less likely to alter the main-chain conformation of the variant [Cunningham and Wells, Science, 244:1081-1085 (1989)]. Alanine is also typically preferred because it is the most common amino acid. Further, it is frequently found in both buried and exposed positions [Creighton, The Proteins, (W.H. Freeman & Co., N.Y.); Chothia, J. Mol. Biol., 150:1 (1976)]. If alanine substitution does not yield adequate amounts of variant, an isoteric amino acid can be used.

Any cysteine residue not involved in maintaining the proper conformation of the anti-TAT antibody or TAT polypeptide also may be substituted, generally with serine, to improve the oxidative stability of the molecule and prevent aberrant crosslinking. Conversely, cysteine bond(s) may be added to the anti-TAT antibody or TAT polypeptide to improve its stability (particularly where the antibody is an antibody fragment such as an Fv fragment).

A particularly preferred type of substitutional variant involves substituting one or more hypervariable region residues of a parent antibody (e.g., a humanized or human antibody). Generally, the resulting variant(s) selected for further development will have improved biological properties relative to the parent antibody from which they are generated. A convenient way for generating such substitutional variants involves affinity maturation using phage display. Briefly, several hypervariable region sites (e.g., 6-7 sites) are mutated to generate all possible amino substitutions at each site. The antibody variants thus generated are displayed in a monovalent fashion from filamentous phage particles as fusions to the gene III product of M13 packaged within each particle. The phage-displayed variants are then screened for their biological activity (e.g., binding affinity) as herein disclosed. In order to identify candidate hypervariable region sites for modification, alanine scanning mutagenesis can be performed to identify hypervariable region residues contributing significantly to antigen binding. Alternatively, or additionally, it may be beneficial to analyze a crystal structure of the antigen-antibody complex to identify contact points between the antibody and human TAT polypeptide. Such contact residues and neighboring residues are candidates for substitution according to the techniques elaborated herein. Once such variants are generated, the panel of variants is subjected to screening as described herein and antibodies with superior properties in one or more relevant assays may be selected for further development.

Nucleic acid molecules encoding amino acid sequence variants of the anti-TAT antibody are prepared by a variety of methods known in the art. These methods include, but are not limited to, isolation from a natural source (in the case of naturally occurring amino acid sequence variants) or preparation by oligonucleotide-mediated (or site-directed) mutagenesis, PCR mutagenesis, and cassette mutagenesis of an earlier prepared variant or a non-variant version of the anti-TAT antibody.

H. Modifications of Anti-TAT Antibodies and TAT Polypeptides

Covalent modifications of anti-TAT antibodies and TAT polypeptides are included within the scope of this invention. One type of covalent modification includes reacting targeted amino acid residues of an anti-TAT antibody or TAT polypeptide with an organic derivatizing agent that is capable of reacting with selected side chains or the N- or C-terminal residues of the anti-TAT antibody or TAT polypeptide. Derivatization with bifunctional agents is useful, for instance, for crosslinking anti-TAT antibody or TAT polypeptide to a water-insoluble support matrix or surface for use in the method for purifying anti-TAT antibodies, and vice-versa. Commonly used crosslinking agents include, e.g., 1,1-bis(diazoacetyl)-2-phenylethane, glutaraldehyde, N-hydroxysuccinimide esters, for example, esters with 4-azidosalicylic acid, homobifunctional imidoesters, including disuccinimidyl esters such as 3,3′-dithiobis(succinidylpropionate), bifunctional maleimides such as bis-N-maleimido-1,8-octane and agents such as methyl-3-[(p-azidophenyl)dithio]propioimidate.

Other modifications include deamidation of glutaminyl and asparaginyl residues to the corresponding glutamyl and aspartyl residues, respectively, hydroxylation of proline and lysine, phosphorylation of hydroxyl groups of seryl or threonyl residues, methylation of the α-amino groups of lysine, arginine, and histidine side chains [T. E. Creighton, Proteins: Structure and Molecular Properties, W.H. Freeman & Co., San Francisco, pp. 79-86 (1983)], acetylation of the N-terminal amine, and amidation of any C-terminal carboxyl group.

Another type of covalent modification of the anti-TAT antibody or TAT polypeptide included within the scope of this invention comprises altering the native glycosylation pattern of the antibody or polypeptide. “Altering the native glycosylation pattern” is intended for purposes herein to mean deleting one or more carbohydrate moieties found in native sequence anti-TAT antibody or TAT polypeptide (either by removing the underlying glycosylation site or by deleting the glycosylation by chemical and/or enzymatic means), and/or adding one or more glycosylation sites that are not present in the native sequence anti-TAT antibody or TAT polypeptide. In addition, the phrase includes qualitative changes in the glycosylation of the native proteins, involving a change in the nature and proportions of the various carbohydrate moieties present.

Glycosylation of antibodies and other polypeptides is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-aceylgalactosamine, galactose, or xylose to a hydroxyamino acid, most commonly serine or threonine, although 5-hydroxyproline or 5-hydroxylysine may also be used.

Addition of glycosylation sites to the anti-TAT antibody or TAT polypeptide is conveniently accomplished by altering the amino acid sequence such that it contains one or more of the above-described tripeptide sequences (for N-linked glycosylation sites). The alteration may also be made by the addition of, or substitution by, one or more serine or threonine residues to the sequence of the original anti-TAT antibody or TAT polypeptide (for O-linked glycosylation sites). The anti-TAT antibody or TAT polypeptide amino acid sequence may optionally be altered through changes at the DNA level, particularly by mutating the DNA encoding the anti-TAT antibody or TAT polypeptide at preselected bases such that codons are generated that will translate into the desired amino acids.

Another means of increasing the number of carbohydrate moieties on the anti-TAT antibody or TAT polypeptide is by chemical or enzymatic coupling of glycosides to the polypeptide. Such methods are described in the art, e.g., in WO 87/05330 published 11 Sep. 1987, and in Aplin and Wriston, CRC Crit. Rev. Biochem., pp. 259-306 (1981).

Removal of carbohydrate moieties present on the anti-TAT antibody or TAT polypeptide may be accomplished chemically or enzymatically or by mutational substitution of codons encoding for amino acid residues that serve as targets for glycosylation. Chemical deglycosylation techniques are known in the art and described, for instance, by Hakimuddin, et al., Arch. Biochem. Biophys., 259:52 (1987) and by Edge et al., Anal. Biochem., 118:131 (1981). Enzymatic cleavage of carbohydrate moieties on polypeptides can be achieved by the use of a variety of endo- and exo-glycosidases as described by Thotakura et al., Meth. Enzymol., 138:350 (1987).

Another type of covalent modification of anti-TAT antibody or TAT polypeptide comprises linking the antibody or polypeptide to one of a variety of nonproteinaceous polymers, e.g., polyethylene glycol (PEG), polypropylene glycol, or polyoxyalkylenes, in the manner set forth in U.S. Pat. Nos. 4,640,835; 4,496,689; 4,301,144; 4,670,417; 4,791,192 or 4,179,337. The antibody or polypeptide also may be entrapped in microcapsules prepared, for example, by coacervation techniques or by interfacial polymerization (for example, hydroxymethylcellulose or gelatin-microcapsules and poly-(methylmethacylate) microcapsules, respectively), in colloidal drug delivery systems (for example, liposomes, albumin microspheres, microemulsions, nano-particles and nanocapsules), or in macroemulsions. Such techniques are disclosed in Remington's Pharmaceutical Sciences, 16th edition, Oslo, A., Ed., (1980).

The anti-TAT antibody or TAT polypeptide of the present invention may also be modified in a way to form chimeric molecules comprising an anti-TAT antibody or TAT polypeptide fused to another, heterologous polypeptide or amino acid sequence.

In one embodiment, such a chimeric molecule comprises a fusion of the anti-TAT antibody or TAT polypeptide with a tag polypeptide which provides an epitope to which an anti-tag antibody can selectively bind. The epitope tag is generally placed at the amino- or carboxyl-terminus of the anti-TAT antibody or TAT polypeptide. The presence of such epitope-tagged forms of the anti-TAT antibody or TAT polypeptide can be detected using an antibody against the tag polypeptide. Also, provision of the epitope tag enables the anti-TAT antibody or TAT polypeptide to be readily purified by affinity purification using an anti-tag antibody or another type of affinity matrix that binds to the epitope tag. Various tag polypeptides and their respective antibodies are well known in the art. Examples include poly-histidine (poly-his) or poly-histidine-glycine (poly-his-gly) tags; the flu HA tag polypeptide and its antibody 12CA5 [Field et al., Mol. Cell. Biol., 8:2159-2165 (1988)]; the c-myc tag and the 8F9, 3C7, 6E10, G4, B7 and 9E10 antibodies thereto [Evan et al., Molecular and Cellular Biology, 5:3610-3616 (1985)]; and the Herpes Simplex virus glycoprotein D (gD) tag and its antibody [Paborsky et al., Protein Engineering, 3(6):547-553 (1990)]. Other tag polypeptides include the Flag-peptide [Hopp et al., BioTechnology 6: 1204-1210 (1988)]; the KT3 epitope peptide [Martin et al., Science, 255:192-194 (1992)]; an α-tubulin epitope peptide [Skinner et al., J. Biol. Chem., 266:15163-15166 (1991)]; and the T7 gene 10 protein peptide tag [Lutz-Freyermuth et al., Proc. Natl. Acad. Sci. USA, 87:6393-6397 (1990)].

In an alternative embodiment, the chimeric molecule may comprise a fusion of the anti-TAT antibody or TAT polypeptide with an immunoglobulin or a particular region of an immunoglobulin. For a bivalent form of the chimeric molecule (also referred to as an “immunoadhesin”), such a fusion could be to the Fc region of an IgG molecule. The Ig fusions preferably include the substitution of a soluble (transmembrane domain deleted or inactivated) form of an anti-TAT antibody or TAT polypeptide in place of at least one variable region within an Ig molecule. In a particularly preferred embodiment, the immunoglobulin fusion includes the hinge, CH₂ and CH₃, or the hinge, CH₁, CH₂ and CH₃ regions of an IgG1 molecule. For the production of immunoglobulin fusions see also U.S. Pat. No. 5,428,130 issued Jun. 27, 1995.

I. Preparation of Anti-TAT Antibodies and TAT Polypeptides

The description below relates primarily to production of anti-TAT antibodies and TAT polypeptides by culturing cells transformed or transfected with a vector containing anti-TAT antibody- and TAT polypeptide-encoding nucleic acid. It is, of course, contemplated that alternative methods, which are well known in the art, may be employed to prepare anti-TAT antibodies and TAT polypeptides. For instance, the appropriate amino acid sequence, or portions thereof, may be produced by direct peptide synthesis using solid-phase techniques [see, e.g., Stewart et al., Solid-Phase Peptide Synthesis, W.H. Freeman Co., San Francisco, Calif. (1969); Merrifield, J. Am. Chem. Soc., 85:2149-2154 (1963)]. In vitro protein synthesis may be performed using manual techniques or by automation. Automated synthesis may be accomplished, for instance, using an Applied Biosystems Peptide Synthesizer (Foster City, Calif.) using manufacturer's instructions. Various portions of the anti-TAT antibody or TAT polypeptide may be chemically synthesized separately and combined using chemical or enzymatic methods to produce the desired anti-TAT antibody or TAT polypeptide.

1. Isolation of DNA Encoding Anti-TAT Antibody or TAT Polypeptide

DNA encoding anti-TAT antibody or TAT polypeptide may be obtained from a cDNA library prepared from tissue believed to possess the anti-TAT antibody or TAT polypeptide mRNA and to express it at a detectable level. Accordingly, human anti-TAT antibody or TAT polypeptide DNA can be conveniently obtained from a cDNA library prepared from human tissue. The anti-TAT antibody- or TAT polypeptide-encoding gene may also be obtained from a genomic library or by known synthetic procedures (e.g., automated nucleic acid synthesis).

Libraries can be screened with probes (such as oligonucleotides of at least about 20-80 bases) designed to identify the gene of interest or the protein encoded by it. Screening the cDNA or genomic library with the selected probe may be conducted using standard procedures, such as described in Sambrook et al., Molecular Cloning: A Laboratory Manual (New York: Cold Spring Harbor Laboratory Press, 1989). An alternative means to isolate the gene encoding anti-TAT antibody or TAT polypeptide is to use PCR methodology [Sambrook et al., supra: Dieffenbach et al., PCR Primer: A Laboratory Manual (Cold Spring Harbor Laboratory Press, 1995)].

Techniques for screening a cDNA library are well known in the art. The oligonucleotide sequences selected as probes should be of sufficient length and sufficiently unambiguous that false positives are minimized. The oligonucleotide is preferably labeled such that it can be detected upon hybridization to DNA in the library being screened. Methods of labeling are well known in the art, and include the use of radiolabels like ³²P-labeled ATP, biotinylation or enzyme labeling. Hybridization conditions, including moderate stringency and high stringency, are provided in Sambrook et al., supra.

Sequences identified in such library screening methods can be compared and aligned to other known sequences deposited and available in public databases such as GenBank or other private sequence databases. Sequence identity (at either the amino acid or nucleotide level) within defined regions of the molecule or across the full-length sequence can be determined using methods known in the art and as described herein.

Nucleic acid having protein coding sequence may be obtained by screening selected cDNA or genomic libraries using the deduced amino acid sequence disclosed herein for the first time, and, if necessary, using conventional primer extension procedures as described in Sambrook et al., supra, to detect precursors and processing intermediates of mRNA that may not have been reverse-transcribed into cDNA.

2. Selection and Transformation of Host Cells

Host cells are transfected or transformed with expression or cloning vectors described herein for anti-TAT antibody or TAT polypeptide production and cultured in conventional nutrient media modified as appropriate for inducing promoters, selecting transformants, or amplifying the genes encoding the desired sequences. The culture conditions, such as media, temperature, pH and the like, can be selected by the skilled artisan without undue experimentation. In general, principles, protocols, and practical techniques for maximizing the productivity of cell cultures can be found in Mammalian Cell Biotechnology: a Practical Approach, M. Butler, ed. (IRL Press, 1991) and Sambrook et al., supra.

Methods of eukaryotic cell transfection and prokaryotic cell transformation are known to the ordinarily skilled artisan, for example, CaCl₂, CaPO₄, liposome-mediated and electroporation. Depending on the host cell used, transformation is performed using standard techniques appropriate to such cells. The calcium treatment employing calcium chloride, as described in Sambrook et al., supra, or electroporation is generally used for prokaryotes. Infection with Agrobacterium tumefaciens is used for transformation of certain plant cells, as described by Shaw et al., Gene, 23:315 (1983) and WO 89/05859 published 29 Jun. 1989. For mammalian cells without such cell walls, the calcium phosphate precipitation method of Graham and van der Eb, Virology, 52:456-457 (1978) can be employed. General aspects of mammalian cell host system transfections have been described in U.S. Pat. No. 4,399,216. Transformations into yeast are typically carried out according to the method of Van Solingen et al., J. Bact., 130:946 (1977) and Hsiao et al., Proc. Natl. Acad. Sci. (USA), 76:3829 (1979). However, other methods for introducing DNA into cells, such as by nuclear microinjection, electroporation, bacterial protoplast fusion with intact cells, or polycations, e.g., polybrene, polyornithine, may also be used. For various techniques for transforming mammalian cells, see Keown et al., Methods in Enzymology, 185:527-537 (1990) and Mansour et al., Nature, 336:348-352 (1988).

Suitable host cells for cloning or expressing the DNA in the vectors herein include prokaryote, yeast, or higher eukaryote cells. Suitable prokaryotes include but are not limited to eubacteria, such as Gram-negative or Gram-positive organisms, for example, Enterobacteriaceae such as E. coli. Various E. coli strains are publicly available, such as E. coli K12 strain MM294 (ATCC 31,446); E. coli X1776 (ATCC 31,537); E. coli strain W3110 (ATCC 27,325) and K5 772 (ATCC 53,635). Other suitable prokaryotic host cells include Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis (e.g., B. licheniformis 41P disclosed in DD 266,710 published 12 Apr. 1989), Pseudomonas such as P. aeruginosa, and Streptomyces. These examples are illustrative rather than limiting. Strain W3110 is one particularly preferred host or parent host because it is a common host strain for recombinant DNA product fermentations. Preferably, the host cell secretes minimal amounts of proteolytic enzymes. For example, strain W3110 may be modified to effect a genetic mutation in the genes encoding proteins endogenous to the host, with examples of such hosts including E. coli W3110 strain 1A2, which has the complete genotype tonA; E. coli W3110 strain 9E4, which has the complete genotype tonA ptr3; E. coli W3110 strain 27C7 (ATCC 55,244), which has the complete genotype tonA ptr3 phoA E15 (argF-lac)169 degP ompT kan^{r}; E. coli W3110 strain 37D6, which has the complete genotype tonA ptr3 phoA E15 (argF-lac)169 degP ompT rbs7 ilvG kan^{r}; E. coli W3110 strain 40B4, which is strain 37D6 with a non-kanamycin resistant degP deletion mutation; and an E. coli strain having mutant periplasmic protease disclosed in U.S. Pat. No. 4,946,783 issued 7 Aug. 1990. Alternatively, in vitro methods of cloning, e.g., PCR or other nucleic acid polymerase reactions, are suitable.

Full length antibody, antibody fragments, and antibody fusion proteins can be produced in bacteria, in particular when glycosylation and Fc effector function are not needed, such as when the therapeutic antibody is conjugated to a cytotoxic agent (e.g., a toxin) and the immunoconjugate by itself shows effectiveness in tumor cell destruction. Full length antibodies have greater half life in circulation. Production in E. coli is faster and more cost efficient. For expression of antibody fragments and polypeptides in bacteria, see, e.g., U.S. Pat. No. 5,648,237 (Carter et. al.), U.S. Pat. No. 5,789,199 (Joly et al.), and U.S. Pat. No. 5,840,523 (Simmons et al.) which describes translation initiation regio (TIR) and signal sequences for optimizing expression and secretion, these patents incorporated herein by reference. After expression, the antibody is isolated from the E. coli cell paste in a soluble fraction and can be purified through, e.g., a protein A or G column depending on the isotype. Final purification can be carried out similar to the process for purifying antibody expressed e.g, in CHO cells.

In addition to prokaryotes, eukaryotic microbes such as filamentous fungi or yeast are suitable cloning or expression hosts for anti-TAT antibody- or TAT polypeptide-encoding vectors. Saccharomyces cerevisiae is a commonly used lower eukaryotic host microorganism. Others include Schizosaccharomyces pombe (Beach and Nurse, Nature, 290: 140 [1981]; EP 139,383 published 2 May 1985); Kluyveromyces hosts (U.S. Pat. No. 4,943,529; Fleer et al., Bio/Technology, 9:968-975 (1991)) such as, e.g., K. lactis (MW98-8C, CBS683, CBS4574; Louvencourt et al., J. Bacteriol., 154(2):737-742 [1983]), K. fragilis (ATCC 12,424), K. bulgaricus (ATCC 16,045), K. wickeramii (ATCC 24,178), K. waltii (ATCC 56,500), K. drosophilarum (ATCC 36,906; Van den Berg et al., Bio/Technology, 8:135 (1990)), K. thermotolerans, and K. marxianus; yarrowia (EP 402,226); Pichia pastoris (EP 183,070; Sreekrishna et al., J. Basic Microbiol., 28:265-278 [1988]); Candida; Trichoderma reesia (EP 244,234); Neurospora crassa (Case et al., Proc. Natl. Acad. Sci. USA, 76:5259-5263 [1979]); Schwanniomyces such as Schwanniomyces occidentalis (EP 394,538 published 31 Oct. 1990); and filamentous fungi such as, e.g., Neurospora, Penicillium, Tolypocladium (WO 91/00357 published 10 Jan. 1991), and Aspergillus hosts such as A. nidulans (Ballance et al., Biochem. Biophys. Res. Commun., 112:284-289 [1983]; Tilburn et al., Gene, 26:205-221 [1983]; Yelton et al., Proc. Natl. Acad. Sci. USA, 81: 1470-1474 [1984]) and A. niger (Kelly and Hynes, EMBO J., 4:475-479 [1985]). Methylotropic yeasts are suitable herein and include, but are not limited to, yeast capable of growth on methanol selected from the genera consisting of Hansenula, Candida, Kloeckera, Pichia, Saccharomyces, Torulopsis, and Rhodotorula. A list of specific species that are exemplary of this class of yeasts may be found in C. Anthony, The Biochemistry of Methylotrophs, 269 (1982).

Suitable host cells for the expression of glycosylated anti-TAT antibody or TAT polypeptide are derived from multicellular organisms. Examples of invertebrate cells include insect cells such as Drosophila S2 and Spodoptera Sf9, as well as plant cells, such as cell cultures of cotton, corn, potato, soybean, petunia, tomato, and tobacco. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruitfly), and Bombyx mori have been identified. A variety of viral strains for transfection are publicly available, e.g., the L-1 variant of Autographa californica NPV and the Bm-5 strain of Bombyx mori NPV, and such viruses may be used as the virus herein according to the present invention, particularly for transfection of Spodoptera frugiperda cells.

However, interest has been greatest in vertebrate cells, and propagation of vertebrate cells in culture (tissue culture) has become a routine procedure. Examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); Chinese hamster ovary cells/-DHFR (CHO, Urlaub et al., Proc. Natl. Acad. Sci. USA77:4216 (1980)); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci. 383:44-68 (1982)); MRC 5 cells; FS4 cells; and a human hepatoma line (Hep G2).

Host cells are transformed with the above-described expression or cloning vectors for anti-TAT antibody or TAT polypeptide production and cultured in conventional nutrient media modified as appropriate for inducing promoters, selecting transformants, or amplifying the genes encoding the desired sequences.

3. Selection and Use of a Replicable Vector

The nucleic acid (e.g., cDNA or genomic DNA) encoding anti-TAT antibody or TAT polypeptide may be inserted into a replicable vector for cloning (amplification of the DNA) or for expression. Various vectors are publicly available. The vector may, for example, be in the form of a plasmid, cosmid, viral particle, or phage. The appropriate nucleic acid sequence may be inserted into the vector by a variety of procedures. In general, DNA is inserted into an appropriate restriction endonuclease site(s) using techniques known in the art. Vector components generally include, but are not limited to, one or more of a signal sequence, an origin of replication, one or more marker genes, an enhancer element, a promoter, and a transcription termination sequence. Construction of suitable vectors containing one or more of these components employs standard ligation techniques which are known to the skilled artisan.

The TAT may be produced recombinantly not only directly, but also as a fusion polypeptide with a heterologous polypeptide, which may be a signal sequence or other polypeptide having a specific cleavage site at the N-terminus of the mature protein or polypeptide. In general, the signal sequence may be a component of the vector, or it may be a part of the anti-TAT antibody- or TAT polypeptide-encoding DNA that is inserted into the vector. The signal sequence may be a prokaryotic signal sequence selected, for example, from the group of the alkaline phosphatase, penicillinase, 1pp, or heat-stable enterotoxin II leaders. For yeast secretion the signal sequence may be, e.g., the yeast invertase leader, alpha factor leader (including Saccharomyces and Kluyveromyces α-factor leaders, the latter described in U.S. Pat. No. 5,010,182), or acid phosphatase leader, the C. albicans glucoamylase leader (EP 362,179 published 4 Apr. 1990), or the signal described in WO 90/13646 published 15 Nov. 1990. In mammalian cell expression, mammalian signal sequences may be used to direct secretion of the protein, such as signal sequences from secreted polypeptides of the same or related species, as well as viral secretory leaders.

Both expression and cloning vectors contain a nucleic acid sequence that enables the vector to replicate in one or more selected host cells. Such sequences are well known for a variety of bacteria, yeast, and viruses. The origin of replication from the plasmid pBR322 is suitable for most Gram-negative bacteria, the 21 plasmid origin is suitable for yeast, and various viral origins (SV40, polyoma, adenovirus, VSV or BPV) are useful for cloning vectors in mammalian cells.

Expression and cloning vectors will typically contain a selection gene, also termed a selectable marker. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media, e.g., the gene encoding D-alanine racemase for Bacilli.

An example of suitable selectable markers for mammalian cells are those that enable the identification of cells competent to take up the anti-TAT antibody- or TAT polypeptide-encoding nucleic acid, such as DHFR or thymidine kinase. An appropriate host cell when wild-type DHFR is employed is the CHO cell line deficient in DHFR activity, prepared and propagated as described by Urlaub et al., Proc. Natl. Acad. Sci. USA, 77:4216 (1980). A suitable selection gene for use in yeast is the trp1 gene present in the yeast plasmid YRp7 [Stinchcomb et al., Nature, 282:39 (1979); Kingsman et al., Gene, 7:141 (1979); Tschemper et al., Gene, 10:157 (1980)]. The trp1 gene provides a selection marker for a mutant strain of yeast lacking the ability to grow in tryptophan, for example, ATCC No. 44076 or PEP4-1 [Jones, Genetics, 85:12 (1977)].

Expression and cloning vectors usually contain a promoter operably linked to the anti-TAT antibody- or TAT polypeptide-encoding nucleic acid sequence to direct mRNA synthesis. Promoters recognized by a variety of potential host cells are well known. Promoters suitable for use with prokaryotic hosts include the β-lactamase and lactose promoter systems [Chang et al., Nature, 275:615 (1978); Goeddel et al., Nature, 281:544 (1979)], alkaline phosphatase, a tryptophan (trp) promoter system [Goeddel, Nucleic Acids Res. 8:4057 (1980); EP 36,776], and hybrid promoters such as the tac promoter [deBoer et al., Proc. Natl. Acad. Sci. USA, 80:21-25 (1983)]. Promoters for use in bacterial systems also will contain a Shine-Dalgarno (S.D.) sequence operably linked to the DNA encoding anti-TAT antibody or TAT polypeptide.

Examples of suitable promoting sequences for use with yeast hosts include the promoters for 3-phosphoglycerate kinase [Hitzeman et al., J. Biol. Chem., 255:2073 (1980)] or other glycolytic enzymes [Hess et al., J. Adv. Enzyme Reg., 7:149 (1968); Holland, Biochemistry, 17:4900 (1978)], such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase.

Other yeast promoters, which are inducible promoters having the additional advantage of transcription controlled by growth conditions, are the promoter regions for alcohol dehydrogenase 2, isocytochrome C, acid phosphatase, degradative enzymes associated with nitrogen metabolism, metallothionein, glyceraldehyde-3-phosphate dehydrogenase, and enzymes responsible for maltose and galactose utilization. Suitable vectors and promoters for use in yeast expression are further described in EP 73,657.

Anti-TAT antibody or TAT polypeptide transcription from vectors in mammalian host cells is controlled, for example, by promoters obtained from the genomes of viruses such as polyoma virus, fowlpox virus (UK 2,211,504 published 5 Jul. 1989), adenovirus (such as Adenovirus 2), bovine papilloma virus, avian sarcoma virus, cytomegalovirus, a retrovirus, hepatitis-B virus and Simian Virus 40 (SV40), from heterologous mammalian promoters, e.g., the actin promoter or an immunoglobulin promoter, and from heat-shock promoters, provided such promoters are compatible with the host cell systems.

Transcription of a DNA encoding the anti-TAT antibody or TAT polypeptide by higher eukaryotes may be increased by inserting an enhancer sequence into the vector. Enhancers are cis-acting elements of DNA, usually about from 10 to 300 bp, that act on a promoter to increase its transcription. Many enhancer sequences are now known from mammalian genes (globin, elastase, albumin, α-fetoprotein, and insulin). Typically, however, one will use an enhancer from a eukaryotic cell virus. Examples include the SV40 enhancer on the late side of the replication origin (bp 100-270), the cytomegalovirus early promoter enhancer, the polyoma enhancer on the late side of the replication origin, and adenovirus enhancers. The enhancer may be spliced into the vector at a position 5′ or 3′ to the anti-TAT antibody or TAT polypeptide coding sequence, but is preferably located at a site 5′ from the promoter.

Expression vectors used in eukaryotic host cells (yeast, fungi, insect, plant, animal, human, or nucleated cells from other multicellular organisms) will also contain sequences necessary for the termination of transcription and for stabilizing the mRNA. Such sequences are commonly available from the 5′ and, occasionally 3′, untranslated regions of eukaryotic or viral DNAs or cDNAs. These regions contain nucleotide segments transcribed as polyadenylated fragments in the untranslated portion of the mRNA encoding anti-TAT antibody or TAT polypeptide.

Still other methods, vectors, and host cells suitable for adaptation to the synthesis of anti-TAT antibody or TAT polypeptide in recombinant vertebrate cell culture are described in Gething et al., Nature, 293:620-625 (1981); Mantei et al., Nature, 281:4046 (1979); EP 117,060; and EP 117,058.

4. Culturing the Host Cells

The host cells used to produce the anti-TAT antibody or TAT polypeptide of this invention may be cultured in a variety of media. Commercially available media such as Ham's F10 (Sigma), Minimal Essential Medium ((MEM), (Sigma), RPMI-1640 (Sigma), and Dulbecco's Modified Eagle's Medium ((DMEM), Sigma) are suitable for culturing the host cells. In addition, any of the media described in Ham et al Meth. Enz. 58:44 (1979), Barnes et al., Anal. Biochem. 102:255 (1980), U.S. Pat. Nos. 4,767,704; 4,657,866; 4,927,762; 4,560,655; or 5,122,469; WO 90/03430; WO 87/00195; or U.S. Pat. Re. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN™ drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

5. Detecting Gene Amplification/Expression

Gene amplification and/or expression may be measured in a sample directly, for example, by conventional Southern blotting, Northern blotting to quantitate the transcription of mRNA [Thomas, Proc. Natl. Acad. Sci. USA, 77:5201-5205 (1980)], dot blotting (DNA analysis), or in situ hybridization, using an appropriately labeled probe, based on the sequences provided herein. Alternatively, antibodies may be employed that can recognize specific duplexes, including DNA duplexes, RNA duplexes, and DNA-RNA hybrid duplexes or DNA-protein duplexes. The antibodies in turn may be labeled and the assay may be carried out where the duplex is bound to a surface, so that upon the formation of duplex on the surface, the presence of antibody bound to the duplex can be detected.

Gene expression, alternatively, may be measured by immunological methods, such as immunohistochemical staining of cells or tissue sections and assay of cell culture or body fluids, to quantitate directly the expression of gene product. Antibodies useful for immunohistochemical staining and/or assay of sample fluids may be either monoclonal or polyclonal, and may be prepared in any mammal. Conveniently, the antibodies may be prepared against a native sequence TAT polypeptide or against a synthetic peptide based on the DNA sequences provided herein or against exogenous sequence fused to TAT DNA and encoding a specific antibody epitope.

6. Purification of Anti-TAT Antibody and TAT Polypeptide

Forms of anti-TAT antibody and TAT polypeptide may be recovered from culture medium or from host cell lysates. If membrane-bound, it can be released from the membrane using a suitable detergent solution (e.g. Triton-X 100) or by enzymatic cleavage. Cells employed in expression of anti-TAT antibody and TAT polypeptide can be disrupted by various physical or chemical means, such as freeze-thaw cycling, sonication, mechanical disruption, or cell lysing agents.

It may be desired to purify anti-TAT antibody and TAT polypeptide from recombinant cell proteins or polypeptides. The following procedures are exemplary of suitable purification procedures: by fractionation on an ion-exchange column; ethanol precipitation; reverse phase HPLC; chromatography on silica or on a cation-exchange resin such as DEAE; chromatofocusing; SDS-PAGE; ammonium sulfate precipitation; gel filtration using, for example, Sephadex G-75; protein A Sepharose columns to remove contaminants such as IgG; and metal chelating columns to bind epitope-tagged forms of the anti-TAT antibody and TAT polypeptide. Various methods of protein purification may be employed and such methods are known in the art and described for example in Deutscher, Methods in Enzymology, 182 (1990); Scopes, Protein Purification: Principles and Practice, Springer-Verlag, New York (1982). The purification step(s) selected will depend, for example, on the nature of the production process used and the particular anti-TAT antibody or TAT polypeptide produced.

When using recombinant techniques, the antibody can be produced intracellularly, in the periplasmic space, or directly secreted into the medium. If the antibody is produced intracellularly, as a first step, the particulate debris, either host cells or lysed fragments, are removed, for example, by centrifugation or ultrafiltration. Carter et al., Bio/Technology 10:163-167 (1992) describe a procedure for isolating antibodies which are secreted to the periplasmic space of E. coli. Briefly, cell paste is thawed in the presence of sodium acetate (pH 3.5), EDTA, and phenylmethylsulfonylfluoride (PMSF) over about 30 min. Cell debris can be removed by centrifugation. Where the antibody is secreted into the medium, supernatants from such expression systems are generally first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants.

The antibody composition prepared from the cells can be purified using, for example, hydroxylapatite chromatography, gel electrophoresis, dialysis, and affinity chromatography, with affinity chromatography being the preferred purification technique. The suitability of protein A as an affinity ligand depends on the species and isotype of any immunoglobulin Fc domain that is present in the antibody. Protein A can be used to purify antibodies that are based on human γ1, γ2 or γ4 heavy chains (Lindmark et al., J. Immunol. Meth. 62:1-13 (1983)). Protein G is recommended for all mouse isotypes and for human γ3 (Guss et al., EMBO J. 5:15671575 (1986)). The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the antibody comprises a C_{H}3 domain, the Bakerbond ABX™ resin (J. T. Baker, Phillipsburg, N.J.) is useful for purification. Other techniques for protein purification such as fractionation on an ion-exchange column, ethanol precipitation, Reverse Phase HPLC, chromatography on silica, chromatography on heparin SEPHAROSE™ chromatography on an anion or cation exchange resin (such as a polyaspartic acid column), chromatofocusing, SDS-PAGE, and ammonium sulfate precipitation are also available depending on the antibody to be recovered.

Following any preliminary purification step(s), the mixture comprising the antibody of interest and contaminants may be subjected to low pH hydrophobic interaction chromatography using an elution buffer at a pH between about 2.5-4.5, preferably performed at low salt concentrations (e.g., from about 0-0.25M salt).

J. Pharmaceutical Formulations

Therapeutic formulations of the anti-TAT antibodies, TAT binding oligopeptides, TAT binding organic molecules and/or TAT polypeptides used in accordance with the present invention are prepared for storage by mixing the antibody, polypeptide, oligopeptide or organic molecule having the desired degree of purity with optional pharmaceutically acceptable carriers, excipients or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers such as acetate, Tris, phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; tonicifiers such as trehalose and sodium chloride; sugars such as sucrose, mannitol, trehalose or sorbitol; surfactant such as polysorbate; salt-forming counter-ions such as sodium; metal complexes (e.g., Zn-protein complexes); and/or non-ionic surfactants such as TWEEN®, PLURONICS® or polyethylene glycol (PEG). The antibody preferably comprises the antibody at a concentration of between 5-200 mg/ml, preferably between 10-100 mg/ml.

The formulations herein may also contain more than one active compound as necessary for the particular indication being treated, preferably those with complementary activities that do not adversely affect each other. For example, in addition to an anti-TAT antibody, TAT binding oligopeptide, or TAT binding organic molecule, it may be desirable to include in the one formulation, an additional antibody, e.g., a second anti-TAT antibody which binds a different epitope on the TAT polypeptide, or an antibody to some other target such as a growth factor that affects the growth of the particular cancer. Alternatively, or additionally, the composition may further comprise a chemotherapeutic agent, cytotoxic agent, cytokine, growth inhibitory agent, anti-hormonal agent, and/or cardioprotectant. Such molecules are suitably present in combination in amounts that are effective for the purpose intended.

The active ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences, 16th edition, Osol, A. Ed. (1980).

Sustained-release preparations may be prepared. Suitable examples of sustained-release preparations include semi-permeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT® (injectable microspheres composed of lactic acid-glycolic acid copolymer and leuprolide acetate), and poly-D-(−)-3-hydroxybutyric acid.

The formulations to be used for in vivo administration must be sterile. This is readily accomplished by filtration through sterile filtration membranes.

K. Diagnosis and Treatment with Anti-TAT Antibodies, TAT Binding Oligopeptides and TAT Binding Organic Molecules

To determine TAT expression in the cancer, various diagnostic assays are available. In one embodiment, TAT polypeptide overexpression may be analyzed by immunohistochemistry (IHC). Parrafin embedded tissue sections from a tumor biopsy may be subjected to the IHC assay and accorded a TAT protein staining intensity criteria as follows:

Score 0—no staining is observed or membrane staining is observed in less than 10% of tumor cells.

Score 1+—a faint/barely perceptible membrane staining is detected in more than 10% of the tumor cells. The cells are only stained in part of their membrane.

Score 2+—a weak to moderate complete membrane staining is observed in more than 10% of the tumor cells.

Score 3+—a moderate to strong complete membrane staining is observed in more than 10% of the tumor cells.

Those tumors with 0 or 1+ scores for TAT polypeptide expression may be characterized as not overexpressing TAT, whereas those tumors with 2+ or 3+ scores may be characterized as overexpressing TAT.

Alternatively, or additionally, FISH assays such as the INFORM® (sold by Ventana, Ariz.) or PATHVISION® (Vysis, Illinois) may be carried out on formalin-fixed, paraffin-embedded tumor tissue to determine the extent (if any) of TAT overexpression in the tumor.

TAT overexpression or amplification may be evaluated using an in vivo diagnostic assay, e.g., by administering a molecule (such as an antibody, oligopeptide or organic molecule) which binds the molecule to be detected and is tagged with a detectable label (e.g., a radioactive isotope or a fluorescent label) and externally scanning the patient for localization of the label.

As described above, the anti-TAT antibodies, oligopeptides and organic molecules of the invention have various non-therapeutic applications. The anti-TAT antibodies, oligopeptides and organic molecules of the present invention can be useful for diagnosis and staging of TAT polypeptide-expressing cancers (e.g., in radioimaging). The antibodies, oligopeptides and organic molecules are also useful for purification or immunoprecipitation of TAT polypeptide from cells, for detection and quantitation of TAT polypeptide in vitro, e.g., in an ELISA or a Western blot, to kill and eliminate TAT-expressing cells from a population of mixed cells as a step in the purification of other cells.

Currently, depending on the stage of the cancer, cancer treatment involves one or a combination of the following therapies: surgery to remove the cancerous tissue, radiation therapy, and chemotherapy. Anti-TAT antibody, oligopeptide or organic molecule therapy may be especially desirable in elderly patients who do not tolerate the toxicity and side effects of chemotherapy well and in metastatic disease where radiation therapy has limited usefulness. The tumor targeting anti-TAT antibodies, oligopeptides and organic molecules of the invention are useful to alleviate TAT-expressing cancers upon initial diagnosis of the disease or during relapse. For therapeutic applications, the anti-TAT antibody, oligopeptide or organic molecule can be used alone, or in combination therapy with, e.g., hormones, antiangiogens, or radiolabelled compounds, or with surgery, cryotherapy, and/or radiotherapy. Anti-TAT antibody, oligopeptide or organic molecule treatment can be administered in conjunction with other forms of conventional therapy, either consecutively with, pre- or post-conventional therapy. Chemotherapeutic drugs such as TAXOTERE® (docetaxel), TAXOL® (palictaxel), estramustine and mitoxantrone are used in treating cancer, in particular, in good risk patients. In the present method of the invention for treating or alleviating cancer, the cancer patient can be administered anti-TAT antibody, oligopeptide or organic molecule in conduction with treatment with the one or more of the preceding chemotherapeutic agents. In particular, combination therapy with palictaxel and modified derivatives (see, e.g., EP0600517) is contemplated. The anti-TAT antibody, oligopeptide or organic molecule will be administered with a therapeutically effective dose of the chemotherapeutic agent. In another embodiment, the anti-TAT antibody, oligopeptide or organic molecule is administered in conjunction with chemotherapy to enhance the activity and efficacy of the chemotherapeutic agent, e.g., paclitaxel. The Physicians' Desk Reference (PDR) discloses dosages of these agents that have been used in treatment of various cancers. The dosing regimen and dosages of these aforementioned chemotherapeutic drugs that are therapeutically effective will depend on the particular cancer being treated, the extent of the disease and other factors familiar to the physician of skill in the art and can be determined by the physician.

In one particular embodiment, a conjugate comprising an anti-TAT antibody, oligopeptide or organic molecule conjugated with a cytotoxic agent is administered to the patient. Preferably, the immunoconjugate bound to the TAT protein is internalized by the cell, resulting in increased therapeutic efficacy of the immunoconjugate in killing the cancer cell to which it binds. In a preferred embodiment, the cytotoxic agent targets or interferes with the nucleic acid in the cancer cell. Examples of such cytotoxic agents are described above and include maytansinoids, calicheamicins, ribonucleases and DNA endonucleases.

The anti-TAT antibodies, oligopeptides, organic molecules or toxin conjugates thereof are administered to a human patient, in accord with known methods, such as intravenous administration, e.g., as a bolus or by continuous infusion over a period of time, by intramuscular, intraperitoneal, intracerobrospinal, subcutaneous, intra-articular, intrasynovial, intrathecal, oral, topical, or inhalation routes. Intravenous or subcutaneous administration of the antibody, oligopeptide or organic molecule is preferred.

Other therapeutic regimens may be combined with the administration of the anti-TAT antibody, oligopeptide or organic molecule. The combined administration includes co-administration, using separate formulations or a single pharmaceutical formulation, and consecutive administration in either order, wherein preferably there is a time period while both (or all) active agents simultaneously exert their biological activities. Preferably such combined therapy results in a synergistic therapeutic effect.

It may also be desirable to combine administration of the anti-TAT antibody or antibodies, oligopeptides or organic molecules, with administration of an antibody directed against another tumor antigen associated with the particular cancer.

In another embodiment, the therapeutic treatment methods of the present invention involves the combined administration of an anti-TAT antibody (or antibodies), oligopeptides or organic molecules and one or more chemotherapeutic agents or growth inhibitory agents, including co-administration of cocktails of different chemotherapeutic agents. Chemotherapeutic agents include estramustine phosphate, prednimustine, cisplatin, 5-fluorouracil, melphalan, cyclophosphamide, hydroxyurea and hydroxyureataxanes (such as paclitaxel and doxetaxel) and/or anthracycline antibiotics. Preparation and dosing schedules for such chemotherapeutic agents may be used according to manufacturers' instructions or as determined empirically by the skilled practitioner. Preparation and dosing schedules for such chemotherapy are also described in Chemotherapy Service Ed., M. C. Perry, Williams & Wilkins, Baltimore, Md. (1992).

The antibody, oligopeptide or organic molecule may be combined with an anti-hormonal compound; e.g., an anti-estrogen compound such as tamoxifen; an anti-progesterone such as onapristone (see, EP 616 812); or an anti-androgen such as flutamide, in dosages known for such molecules. Where the cancer to be treated is androgen independent cancer, the patient may previously have been subjected to anti-androgen therapy and, after the cancer becomes androgen independent, the anti-TAT antibody, oligopeptide or organic molecule (and optionally other agents as described herein) may be administered to the patient.

Sometimes, it may be beneficial to also co-administer a cardioprotectant (to prevent or reduce myocardial dysfunction associated with the therapy) or one or more cytokines to the patient. In addition to the above therapeutic regimes, the patient may be subjected to surgical removal of cancer cells and/or radiation therapy, before, simultaneously with, or post antibody, oligopeptide or organic molecule therapy. Suitable dosages for any of the above co-administered agents are those presently used and may be lowered due to the combined action (synergy) of the agent and anti-TAT antibody, oligopeptide or organic molecule.

For the prevention or treatment of disease, the dosage and mode of administration will be chosen by the physician according to known criteria. The appropriate dosage of antibody, oligopeptide or organic molecule will depend on the type of disease to be treated, as defined above, the severity and course of the disease, whether the antibody, oligopeptide or organic molecule is administered for preventive or therapeutic purposes, previous therapy, the patient's clinical history and response to the antibody, oligopeptide or organic molecule, and the discretion of the attending physician. The antibody, oligopeptide or organic molecule is suitably administered to the patient at one time or over a series of treatments. Preferably, the antibody, oligopeptide or organic molecule is administered by intravenous infusion or by subcutaneous injections. Depending on the type and severity of the disease, about 1 μg/kg to about 50 mg/kg body weight (e.g., about 0.1-15 mg/kg/dose) of antibody can be an initial candidate dosage for administration to the patient, whether, for example, by one or more separate administrations, or by continuous infusion. A dosing regimen can comprise administering an initial loading dose of about 4 mg/kg, followed by a weekly maintenance dose of about 2 mg/kg of the anti-TAT antibody. However, other dosage regimens may be useful. A typical daily dosage might range from about 1 μg/kg to 100 mg/kg or more, depending on the factors mentioned above. For repeated administrations over several days or longer, depending on the condition, the treatment is sustained until a desired suppression of disease symptoms occurs. The progress of this therapy can be readily monitored by conventional methods and assays and based on criteria known to the physician or other persons of skill in the art.

Aside from administration of the antibody protein to the patient, the present application contemplates administration of the antibody by gene therapy. Such administration of nucleic acid encoding the antibody is encompassed by the expression “administering a therapeutically effective amount of an antibody”. See, for example, WO96/07321 published Mar. 14, 1996 concerning the use of gene therapy to generate intracellular antibodies.

There are two major approaches to getting the nucleic acid (optionally contained in a vector) into the patient's cells; in vivo and ex vivo. For in vivo delivery the nucleic acid is injected directly into the patient, usually at the site where the antibody is required. For ex vivo treatment, the patient's cells are removed, the nucleic acid is introduced into these isolated cells and the modified cells are administered to the patient either directly or, for example, encapsulated within porous membranes which are implanted into the patient (see, e.g., U.S. Pat. Nos. 4,892,538 and 5,283,187). There are a variety of techniques available for introducing nucleic acids into viable cells. The techniques vary depending upon whether the nucleic acid is transferred into cultured cells in vitro, or in vivo in the cells of the intended host. Techniques suitable for the transfer of nucleic acid into mammalian cells in vitro include the use of liposomes, electroporation, microinjection, cell fusion, DEAE-dextran, the calcium phosphate precipitation method, etc. A commonly used vector for ex vivo delivery of the gene is a retroviral vector.

The currently preferred in vivo nucleic acid transfer techniques include transfection with viral vectors (such as adenovirus, Herpes simplex I virus, or adeno-associated virus) and lipid-based systems (useful lipids for lipid-mediated transfer of the gene are DOTMA, DOPE and DC-Chol, for example). For review of the currently known gene marking and gene therapy protocols see Anderson et al., Science 256:808-813 (1992). See also WO 93/25673 and the references cited therein.

The anti-TAT antibodies of the invention can be in the different forms encompassed by the definition of “antibody” herein. Thus, the antibodies include full length or intact antibody, antibody fragments, native sequence antibody or amino acid variants, humanized, chimeric or fusion antibodies, immunoconjugates, and functional fragments thereof. In fusion antibodies an antibody sequence is fused to a heterologous polypeptide sequence. The antibodies can be modified in the Fc region to provide desired effector functions. As discussed in more detail in the sections herein, with the appropriate Fc regions, the naked antibody bound on the cell surface can induce cytotoxicity, e.g., via antibody-dependent cellular cytotoxicity (ADCC) or by recruiting complement in complement dependent cytotoxicity, or some other mechanism. Alternatively, where it is desirable to eliminate or reduce effector function, so as to minimize side effects or therapeutic complications, certain other Fc regions may be used.

In one embodiment, the antibody competes for binding or bind substantially to, the same epitope as the antibodies of the invention. Antibodies having the biological characteristics of the present anti-TAT antibodies of the invention are also contemplated, specifically including the in vivo tumor targeting and any cell proliferation inhibition or cytotoxic characteristics.

Methods of producing the above antibodies are described in detail herein.

The present anti-TAT antibodies, oligopeptides and organic molecules are useful for treating a TAT-expressing cancer or alleviating one or more symptoms of the cancer in a mammal. Such a cancer includes prostate cancer, cancer of the urinary tract, lung cancer, breast cancer, colon cancer and ovarian cancer, more specifically, prostate adenocarcinoma, renal cell carcinomas, colorectal adenocarcinomas, lung adenocarcinomas, lung squamous cell carcinomas, and pleural mesothelioma. The cancers encompass metastatic cancers of any of the preceding. The antibody, oligopeptide or organic molecule is able to bind to at least a portion of the cancer cells that express TAT polypeptide in the mammal. In a preferred embodiment, the antibody, oligopeptide or organic molecule is effective to destroy or kill TAT-expressing tumor cells or inhibit the growth of such tumor cells, in vitro or in vivo, upon binding to TAT polypeptide on the cell. Such an antibody includes a naked anti-TAT antibody (not conjugated to any agent). Naked antibodies that have cytotoxic or cell growth inhibition properties can be further harnessed with a cytotoxic agent to render them even more potent in tumor cell destruction. Cytotoxic properties can be conferred to an anti-TAT antibody by, e.g., conjugating the antibody with a cytotoxic agent, to form an immunoconjugate as described herein. The cytotoxic agent or a growth inhibitory agent is preferably a small molecule. Toxins such as calicheamicin or a maytansinoid and analogs or derivatives thereof, are preferable.

The invention provides a composition comprising an anti-TAT antibody, oligopeptide or organic molecule of the invention, and a carrier. For the purposes of treating cancer, compositions can be administered to the patient in need of such treatment, wherein the composition can comprise one or more anti-TAT antibodies present as an immunoconjugate or as the naked antibody. In a further embodiment, the compositions can comprise these antibodies, oligopeptides or organic molecules in combination with other therapeutic agents such as cytotoxic or growth inhibitory agents, including chemotherapeutic agents. The invention also provides formulations comprising an anti-TAT antibody, oligopeptide or organic molecule of the invention, and a carrier. In one embodiment, the formulation is a therapeutic formulation comprising a pharmaceutically acceptable carrier.

Another aspect of the invention is isolated nucleic acids encoding the anti-TAT antibodies. Nucleic acids encoding both the H and L chains and especially the hypervariable region residues, chains which encode the native sequence antibody as well as variants, modifications and humanized versions of the antibody, are encompassed.

The invention also provides methods useful for treating a TAT polypeptide-expressing cancer or alleviating one or more symptoms of the cancer in a mammal, comprising administering a therapeutically effective amount of an anti-TAT antibody, oligopeptide or organic molecule to the mammal. The antibody, oligopeptide or organic molecule therapeutic compositions can be administered short term (acute) or chronic, or intermittent as directed by physician. Also provided are methods of inhibiting the growth of, and killing a TAT polypeptide-expressing cell.

The invention also provides kits and articles of manufacture comprising at least one anti-TAT antibody, oligopeptide or organic molecule. Kits containing anti-TAT antibodies, oligopeptides or organic molecules find use, e.g., for TAT cell killing assays, for purification or immunoprecipitation of TAT polypeptide from cells. For example, for isolation and purification of TAT, the kit can contain an anti-TAT antibody, oligopeptide or organic molecule coupled to beads (e.g., sepharose beads). Kits can be provided which contain the antibodies, oligopeptides or organic molecules for detection and quantitation of TAT in vitro, e.g., in an ELISA or a Western blot. Such antibody, oligopeptide or organic molecule useful for detection may be provided with a label such as a fluorescent or radiolabel.

L. Articles of Manufacture and Kits

Another embodiment of the invention is an article of manufacture containing materials useful for the treatment of anti-TAT expressing cancer. The article of manufacture comprises a container and a label or package insert on or associated with the container. Suitable containers include, for example, bottles, vials, syringes, etc. The containers may be formed from a variety of materials such as glass or plastic. The container holds a composition which is effective for treating the cancer condition and may have a sterile access port (for example the container may be an intravenous solution bag or a vial having a stopper pierceable by a hypodermic injection needle). At least one active agent in the composition is an anti-TAT antibody, oligopeptide or organic molecule of the invention. The label or package insert indicates that the composition is used for treating cancer. The label or package insert will further comprise instructions for administering the antibody, oligopeptide or organic molecule composition to the cancer patient. Additionally, the article of manufacture may further comprise a second container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

Kits are also provided that are useful for various purposes, e.g., for TAT-expressing cell killing assays, for purification or immunoprecipitation of TAT polypeptide from cells. For isolation and purification of TAT polypeptide, the kit can contain an anti-TAT antibody, oligopeptide or organic molecule coupled to beads (e.g., sepharose beads). Kits can be provided which contain the antibodies, oligopeptides or organic molecules for detection and quantitation of TAT polypeptide in vitro, e.g., in an ELISA or a Western blot. As with the article of manufacture, the kit comprises a container and a label or package insert on or associated with the container. The container holds a composition comprising at least one anti-TAT antibody, oligopeptide or organic molecule of the invention. Additional containers may be included that contain, e.g., diluents and buffers, control antibodies. The label or package insert may provide a description of the composition as well as instructions for the intended in vitro or diagnostic use.

M. Uses for TAT Polypeptides and TAT-Polypeptide Encoding Nucleic Acids

Nucleotide sequences (or their complement) encoding TAT polypeptides have various applications in the art of molecular biology, including uses as hybridization probes, in chromosome and gene mapping and in the generation of anti-sense RNA and DNA probes. TAT-encoding nucleic acid will also be useful for the preparation of TAT polypeptides by the recombinant techniques described herein, wherein those TAT polypeptides may find use, for example, in the preparation of anti-TAT antibodies as described herein.

The full-length native sequence TAT gene, or portions thereof, may be used as hybridization probes for a cDNA library to isolate the full-length TAT cDNA or to isolate still other cDNAs (for instance, those encoding naturally-occurring variants of TAT or TAT from other species) which have a desired sequence identity to the native TAT sequence disclosed herein. Optionally, the length of the probes will be about 20 to about 50 bases. The hybridization probes may be derived from at least partially novel regions of the full length native nucleotide sequence wherein those regions may be determined without undue experimentation or from genomic sequences including promoters, enhancer elements and introns of native sequence TAT. By way of example, a screening method will comprise isolating the coding region of the TAT gene using the known DNA sequence to synthesize a selected probe of about 40 bases. Hybridization probes may be labeled by a variety of labels, including radionucleotides such as ³²P or ³⁵S, or enzymatic labels such as alkaline phosphatase coupled to the probe via avidin/biotin coupling systems. Labeled probes having a sequence complementary to that of the TAT gene of the present invention can be used to screen libraries of human cDNA, genomic DNA or mRNA to determine which members of such libraries the probe hybridizes to. Hybridization techniques are described in further detail in the Examples below. Any EST sequences disclosed in the present application may similarly be employed as probes, using the methods disclosed herein.

Other useful fragments of the TAT-encoding nucleic acids include antisense or sense oligonucleotides comprising a singe-stranded nucleic acid sequence (either RNA or DNA) capable of binding to target TAT mRNA (sense) or TAT DNA (antisense) sequences. Antisense or sense oligonucleotides, according to the present invention, comprise a fragment of the coding region of TAT DNA. Such a fragment generally comprises at least about 14 nucleotides, preferably from about 14 to 30 nucleotides. The ability to derive an antisense or a sense oligonucleotide, based upon a cDNA sequence encoding a given protein is described in, for example, Stein and Cohen (Cancer Res. 48:2659, 1988) and van der Krol et al. (BioTechniques 6:958, 1988).

Binding of antisense or sense oligonucleotides to target nucleic acid sequences results in the formation of duplexes that block transcription or translation of the target sequence by one of several means, including enhanced degradation of the duplexes, premature termination of transcription or translation, or by other means. Such methods are encompassed by the present invention. The antisense oligonucleotides thus may be used to block expression of TAT proteins, wherein those TAT proteins may play a role in the induction of cancer in mammals. Antisense or sense oligonucleotides further comprise oligonucleotides having modified sugar-phosphodiester backbones (or other sugar linkages, such as those described in WO 91/06629) and wherein such sugar linkages are resistant to endogenous nucleases. Such oligonucleotides with resistant sugar linkages are stable in vivo (i.e., capable of resisting enzymatic degradation) but retain sequence specificity to be able to bind to target nucleotide sequences.

Preferred intragenic sites for antisense binding include the region incorporating the translation initiation/start codon (5′-AUG/5′-ATG) or termination/stop codon (5′-UAA, 5′-UAG and 5-UGA/5′-TAA, 5′-TAG and 5′-TGA) of the open reading frame (ORF) of the gene. These regions refer to a portion of the mRNA or gene that encompasses from about 25 to about 50 contiguous nucleotides in either direction (i.e., 5′ or 3′) from a translation initiation or termination codon. Other preferred regions for antisense binding include: introns; exons; intron-exon junctions; the open reading frame (ORF) or “coding region,” which is the region between the translation initiation codon and the translation termination codon; the 5′ cap of an mRNA which comprises an N7-methylated guanosine residue joined to the 5′-most residue of the mRNA via a 5′-5′ triphosphate linkage and includes 5′ cap structure itself as well as the first 50 nucleotides adjacent to the cap; the 5′ untranslated region (5′UTR), the portion of an mRNA in the 5′ direction from the translation initiation codon, and thus including nucleotides between the 5′ cap site and the translation initiation codon of an mRNA or corresponding nucleotides on the gene; and the 3′ untranslated region (3′UTR), the portion of an mRNA in the 3′ direction from the translation termination codon, and thus including nucleotides between the translation termination codon and 3′ end of an mRNA or corresponding nucleotides on the gene.

Specific examples of preferred antisense compounds useful for inhibiting expression of TAT proteins include oligonucleotides containing modified backbones or non-natural internucleoside linkages. Oligonucleotides having modified backbones include those that retain a phosphorus atom in the backbone and those that do not have a phosphorus atom in the backbone. For the purposes of this specification, and as sometimes referenced in the art, modified oligonucleotides that do not have a phosphorus atom in their internucleoside backbone can also be considered to be oligonucleosides. Preferred modified oligonucleotide backbones include, for example, phosphorothioates, chiral phosphorothioates, phosphorodithioates, phosphotriesters, aminoalkylphosphotri-esters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates and borano-phosphates having normal 3′-5′ linkages, 2′-5′ linked analogs of these, and those having inverted polarity wherein one or more internucleotide linkages is a 3′ to 3′, 5′ to 5′ or 2′ to 2′ linkage. Preferred oligonucleotides having inverted polarity comprise a single 3′ to 3′ linkage at the 3′-most internucleotide linkage i.e. a single inverted nucleoside residue which may be abasic (the nucleobase is missing or has a hydroxyl group in place thereof). Various salts, mixed salts and free acid forms are also included. Representative United States patents that teach the preparation of phosphorus-containing linkages include, but are not limited to, U.S. Pat. Nos.: 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; 5,194,599; 5,565,555; 5,527,899; 5,721,218; 5,672,697 and 5,625,050, each of which is herein incorporated by reference.

Preferred modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH.sub.2 component parts. Representative United States patents that teach the preparation of such oligonucleosides include, but are not limited to, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; 5,792,608; 5,646,269 and 5,677,439, each of which is herein incorporated by reference.

In other preferred antisense oligonucleotides, both the sugar and the internucleoside linkage, i.e., the backbone, of the nucleotide units are replaced with novel groups. The base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, in particular an aminoethylglycine backbone. The nucleobases are retained and are bound directly or indirectly to aza nitrogen atoms of the amide portion of the backbone. Representative United States patents that teach the preparation of PNA compounds include, but are not limited to, U.S. Pat. Nos.: 5,539,082; 5,714,331; and 5,719,262, each of which is herein incorporated by reference. Further teaching of PNA compounds can be found in Nielsen et al., Science, 1991, 254, 1497-1500.

Preferred antisense oligonucleotides incorporate phosphorothioate backbones and/or heteroatom backbones, and in particular —CH₂—NH—O—CH₂—, —CH₂—N(CH₃)—O—CH₂— [known as a methylene (methylimino) or MMI backbone], —CH₂—O—N(CH₃)—CH₂—, —CH₂—N(CH₃)—N(CH₃)—CH₂— and —O—N(CH₃)—CH₂—CH₂— [wherein the native phosphodiester backbone is represented as —O—P—O—CH₂—] described in the above referenced U.S. Pat. No. 5,489,677, and the amide backbones of the above referenced U.S. Pat. No. 5,602,240. Also preferred are antisense oligonucleotides having morpholino backbone structures of the above-referenced U.S. Pat. No. 5,034,506.

Modified oligonucleotides may also contain one or more substituted sugar moieties. Preferred oligonucleotides comprise one of the following at the 2′ position: OH; P; O-alkyl, S-alkyl, or N-alkyl; O-alkenyl, S-alkenyl, or N-alkenyl; O-alkynyl, S-alkynl or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Particularly preferred are O[(CH₂)ₙO]ₘCH₃, O(CH₂)ₙOCH₃, O(CH₂)ₙNH₂, O(CH₂)ₙCH₃, O(CH₂)ₙONH₂, and O(CH₂)ₙON[(CH₂)ₙCH₃)]₂, where n and m are from 1 to about 10. Other preferred antisense oligonucleotides comprise one of the following at the 2′ position: C₁ to C₁₀ lower alkyl, substituted lower alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂ CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. A preferred modification includes 2′-methoxyethoxy (2′-O—CH₂CH₂OCH₃, also known as 2′-O-(2-methoxyethyl) or 2′-MOE) (Martin et al., Helv. Chim. Acta, 1995, 78, 486-504) i.e., an alkoxyalkoxy group. A further preferred modification includes 2′-dimethylaminooxyethoxy, i.e., a O(CH₂)₂ON(CH₃)₂ group, also known as 2′-DMAOE, as described in examples hereinbelow, and 2′-dimethylaminoethoxyethoxy (also known in the art as 2′-O-dimethylaminoethoxyethyl or 2′-DMAEOE), i.e., 2′-O—CH₂—O—CH₂—N(CH₂).

A further prefered modification includes Locked Nucleic Acids (LNAs) in which the 2′-hydroxyl group is linked to the 3′ or 4′ carbon atom of the sugar ring thereby forming a bicyclic sugar moiety. The linkage is preferably a methelyne (—CH₂—)ₙ group bridging the 2′ oxygen atom and the 4′ carbon atom wherein n is 1 or 2. LNAs and preparation thereof are described in WO 98/39352 and WO 99/14226.

Other preferred modifications include 2′-methoxy (2′-O—CH₃), 2′-aminopropoxy (2′-OCH₂CH₂CH₂NH₂), 2′-allyl (2′-CH₂—CH═CH₂), 2′-O-allyl (2′-O—CH₂—CH═CH₂) and 2′-fluoro (2′-F). The 2′-modification may be in the arabino (up) position or ribo (down) position. A preferred 2′-arabino modification is 2′-F. Similar modifications may also be made at other positions on the oligonucleotide, particularly the 3′ position of the sugar on the 3′ terminal nucleotide or in 2′-5′ linked oligonucleotides and the 5′ position of 5′ terminal nucleotide. Oligonucleotides may also have sugar mimetics such as cyclobutyl moieties in place of the pentofuranosyl sugar. Representative United States patents that teach the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; and 5,700,920, each of which is herein incorporated by reference in its entirety.

Oligonucleotides may also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C═C—CH₃ or —CH₂—C═CH) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified nucleobases include tricyclic pyrimidines such as phenoxazine cytidine(1H-pyrimido[5,4-b][1,4]benzoxazin-2(3H)-one), phenothiazine cytidine (1H-pyrimido[5,4-b][1,4]benzothiazin-2(3H)-one), G-clamps such as a substituted phenoxazine cytidine (e.g. 9-(2-aminoethoxy)-H-pyrimido[5,4-b][1,4]benzoxazin-2(3H)-one), carbazole cytidine (2H-pyrimido[4,5-b]indol-2-one), pyridoindole cytidine (H-pyrido[3′,2′:4,5]pyrrolo[2,3-d]pyrimidin-2-one). Modified nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, and those disclosed by Englisch et al., Angewandte Chemie, International Edition, 1991, 30, 613. Certain of these nucleobases are particularly useful for increasing the binding affinity of the oligomeric compounds of the invention. These include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2.degree. C. (Sanghvi et al, Antisense Research and Applications, CRC Press, Boca Raton, 1993, pp. 276-278) and are preferred base substitutions, even more particularly when combined with 2′-O-methoxyethyl sugar modifications. Representative United States patents that teach the preparation of modified nucleobases include, but are not limited to: U.S. Pat. No. 3,687,808, as well as U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,645,985; 5,830,653; 5,763,588; 6,005,096; 5,681,941 and 5,750,692, each of which is herein incorporated by reference.

Another modification of antisense oligonucleotides chemically linking to the oligonucleotide one or more moieties or conjugates which enhance the activity, cellular distribution or cellular uptake of the oligonucleotide. The compounds of the invention can include conjugate groups covalently bound to functional groups such as primary or secondary hydroxyl groups. Conjugate groups of the invention include intercalators, reporter molecules, polyamines, polyamides, polyethylene glycols, polyethers, groups that enhance the pharmacodynamic properties of oligomers, and groups that enhance the pharmacokinetic properties of oligomers. Typical conjugates groups include cholesterols, lipids, cation lipids, phospholipids, cationic phospholipids, biotin, phenazine, folate, phenanthridine, anthraquinone, acridine, fluoresceins, rhodamines, coumarins, and dyes. Groups that enhance the pharmacodynamic properties, in the context of this invention, include groups that improve oligomer uptake, enhance oligomer resistance to degradation, and/or strengthen sequence-specific hybridization with RNA. Groups that enhance the pharmacokinetic properties, in the context of this invention, include groups that improve oligomer uptake, distribution, metabolism or excretion. Conjugate moieties include but are not limited to lipid moieties such as a cholesterol moiety (Letsinger et al., Proc. Natl. Acad. Sci. USA, 1989, 86, 6553-6556), cholic acid (Manoharan et al., Bioorg. Med. Chem. Let., 1994, 4, 1053-1060), a thioether, e.g., hexyl-S-tritylthiol (Manoharan et al., Ann. N.Y. Acad. Sci., 1992, 660, 306-309; Manoharan et al., Bioorg. Med. Chem. Let., 1993, 3, 2765-2770), a thiocholesterol (Oberhauser et al., Nucl. Acids Res., 1992, 20, 533-538), an aliphatic chain, e.g., dodecandiol or undecyl residues (Saison-Behmoaras et al., EMBO J., 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethyl-ammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid (Manoharan et al., Tetrahedron Lett., 1995, 36, 3651-3654), a palmityl moiety (Mishra et al., Biochim. Biophys. Acta, 1995, 1264, 229-237), or an octadecylamine or hexylamino-carbonyl-oxycholesterol moiety. Oligonucleotides of the invention may also be conjugated to active drug substances, for example, aspirin, warfarin, phenylbutazone, ibuprofen, suprofen, fenbufen, ketoprofen, (S)-(+)-pranoprofen, carprofen, dansylsarcosine, 2,3,5-triiodobenzoic acid, flufenamic acid, folinic acid, a benzothiadiazide, chlorothiazide, a diazepine, indomethicin, a barbiturate, a cephalosporin, a sulfa drug, an antidiabetic, an antibacterial or an antibiotic. Oligonucleotide-drug conjugates and their preparation are described in U.S. patent application Ser. No. 09/334,130 (filed Jun. 15, 1999) and U.S. Pat. Nos.: 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941, each of which is herein incorporated by reference.

It is not necessary for all positions in a given compound to be uniformly modified, and in fact more than one of the aforementioned modifications may be incorporated in a single compound or even at a single nucleoside within an oligonucleotide. The present invention also includes antisense compounds which are chimeric compounds. “Chimeric” antisense compounds or “chimeras,” in the context of this invention, are antisense compounds, particularly oligonucleotides, which contain two or more chemically distinct regions, each made up of at least one monomer unit, i.e., a nucleotide in the case of an oligonucleotide compound. These oligonucleotides typically contain at least one region wherein the oligonucleotide is modified so as to confer upon the oligonucleotide increased resistance to nuclease degradation, increased cellular uptake, and/or increased binding affinity for the target nucleic acid. An additional region of the oligonucleotide may serve as a substrate for enzymes capable of cleaving RNA:DNA or RNA:RNA hybrids. By way of example, RNase H is a cellular endonuclease which cleaves the RNA strand of an RNA:DNA duplex. Activation of RNase H, therefore, results in cleavage of the RNA target, thereby greatly enhancing the efficiency of oligonucleotide inhibition of gene expression. Consequently, comparable results can oftenbe obtained with shorter oligonucleotides when chimeric oligonucleotides are used, compared to phosphorothioate deoxyoligonucleotides hybridizing to the same target region. Chimeric antisense compounds of the invention may be formed as composite structures of two or more oligonucleotides, modified oligonucleotides, oligonucleosides and/or oligonucleotide mimetics as described above. Preferred chimeric antisense oligonucleotides incorporate at least one 2′ modified sugar (preferably 2′-O—(CH₂)₂—O—CH₃) at the 3′ terminal to confer nuclease resistance and a region with at least 4 contiguous 2′-H sugars to confer RNase H activity. Such compounds have also been referred to in the art as hybrids or gapmers. Preferred gapmers have a region of 2′ modified sugars (preferably 2′-O—(CH₂)₂—O—CH₃) at the 3′-terminal and at the 5′ terminal separated by at least one region having at least 4 contiguous 2′-H sugars and preferably incorporate phosphorothioate backbone linkages. Representative United States patents that teach the preparation of such hybrid structures include, but are not limited to, U.S. Pat. Nos. 5,013,830; 5,149,797; 5,220,007; 5,256,775; 5,366,878; 5,403,711; 5,491,133; 5,565,350; 5,623,065; 5,652,355; 5,652,356; and 5,700,922, each of which is herein incorporated by reference in its entirety.

The antisense compounds used in accordance with this invention may be conveniently and routinely made through the well-known technique of solid phase synthesis. Equipment for such synthesis is sold by several vendors including, for example, Applied Biosystems (Foster City, Calif.). Any other means for such synthesis known in the art may additionally or alternatively be employed. It is well known to use similar techniques to prepare oligonucleotides such as the phosphorothioates and alkylated derivatives. The compounds of the invention may also be admixed, encapsulated, conjugated or otherwise associated with other molecules, molecule structures or mixtures of compounds, as for example, liposomes, receptor targeted molecules, oral, rectal, topical or other formulations, for assisting in uptake, distribution and/or absorption. Representative United States patents that teach the preparation of such uptake, distribution and/or absorption assisting formulations include, but are not limited to, U.S. Pat. Nos. 5,108,921; 5,354,844; 5,416,016; 5,459,127; 5,521,291; 5,543,158; 5,547,932; 5,583,020; 5,591,721; 4,426,330; 4,534,899; 5,013,556; 5,108,921; 5,213,804; 5,227,170; 5,264,221; 5,356,633; 5,395,619; 5,416,016; 5,417,978; 5,462,854; 5,469,854; 5,512,295; 5,527,528; 5,534,259; 5,543,152; 5,556,948; 5,580,575; and 5,595,756, each of which is herein incorporated by reference.

Other examples of sense or antisense oligonucleotides include those oligonucleotides which are covalently linked to organic moieties, such as those described in WO 90/10048, and other moieties that increases affinity of the oligonucleotide for a target nucleic acid sequence, such as poly-(L-lysine). Further still, intercalating agents, such as ellipticine, and alkylating agents or metal complexes may be attached to sense or antisense oligonucleotides to modify binding specificities of the antisense or sense oligonucleotide for the target nucleotide sequence.

Antisense or sense oligonucleotides may be introduced into a cell containing the target nucleic acid sequence by any gene transfer method, including, for example, CaPO₄-mediated DNA transfection, electroporation, or by using gene transfer vectors such as Epstein-Barr virus. In a preferred procedure, an antisense or sense oligonucleotide is inserted into a suitable retroviral vector. A cell containing the target nucleic acid sequence is contacted with the recombinant retroviral vector, either in vivo or ex vivo. Suitable retroviral vectors include, but are not limited to, those derived from the murine retrovirus M-MuLV, N2 (a retrovirus derived from M-MuLV), or the double copy vectors designated DCT5A, DCT5B and DCT5C (see WO 90/13641).

Sense or antisense oligonucleotides also may be introduced into a cell containing the target nucleotide sequence by formation of a conjugate with a ligand binding molecule, as described in WO 91/04753. Suitable ligand binding molecules include, but are not limited to, cell surface receptors, growth factors, other cytokines, or other ligands that bind to cell surface receptors. Preferably, conjugation of the ligand binding molecule does not substantially interfere with the ability of the ligand binding molecule to bind to its corresponding molecule or receptor, or block entry of the sense or antisense oligonucleotide or its conjugated version into the cell.

Alternatively, a sense or an antisense oligonucleotide may be introduced into a cell containing the target nucleic acid sequence by formation of an oligonucleotide-lipid complex, as described in WO 90/10448. The sense or antisense oligonucleotide-lipid complex is preferably dissociated within the cell by an endogenous lipase.

Antisense or sense RNA or DNA molecules are generally at least about 5 nucleotides in length, alternatively at least about 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 nucleotides in length, wherein in this context the term “about” means the referenced nucleotide sequence length plus or minus 10% of that referenced length.

The probes may also be employed in PCR techniques to generate a pool of sequences for identification of closely related TAT coding sequences.

Nucleotide sequences encoding a TAT can also be used to construct hybridization probes for mapping the gene which encodes that TAT and for the genetic analysis of individuals with genetic disorders. The nucleotide sequences provided herein may be mapped to a chromosome and specific regions of a chromosome using known techniques, such as in situ hybridization, linkage analysis against known chromosomal markers, and hybridization screening with libraries.

When the coding sequences for TAT encode a protein which binds to another protein (example, where the TAT is a receptor), the TAT can be used in assays to identify the other proteins or molecules involved in the binding interaction. By such methods, inhibitors of the receptor/ligand binding interaction can be identified. Proteins involved in such binding interactions can also be used to screen for peptide or small molecule inhibitors or agonists of the binding interaction. Also, the receptor TAT can be used to isolate correlative ligand(s). Screening assays can be designed to find lead compounds that mimic the biological activity of a native TAT or a receptor for TAT. Such screening assays will include assays amenable to high-throughput screening of chemical libraries, making them particularly suitable for identifying small molecule drug candidates. Small molecules contemplated include synthetic organic or inorganic compounds. The assays can be performed in a variety of formats, including protein-protein binding assays, biochemical screening assays, immunoassays and cell based assays, which are well characterized in the art.

Nucleic acids which encode TAT or its modified forms can also be used to generate either transgenic animals or “knock out” animals which, in turn, are useful in the development and screening of therapeutically useful reagents. A transgenic animal (e.g., a mouse or rat) is an animal having cells that contain a transgene, which transgene was introduced into the animal or an ancestor of the animal at a prenatal, e.g., an embryonic stage. A transgene is a DNA which is integrated into the genome of a cell from which a transgenic animal develops. In one embodiment, cDNA encoding TAT can be used to clone genomic DNA encoding TAT in accordance with established techniques and the genomic sequences used to generate transgenic animals that contain cells which express DNA encoding TAT. Methods for generating transgenic animals, particularly animals such as mice or rats, have become conventional in the art and are described, for example, in U.S. Pat. Nos. 4,736,866 and 4,870,009. Typically, particular cells would be targeted for TAT transgene incorporation with tissue-specific enhancers. Transgenic animals that include a copy of a transgene encoding TAT introduced into the germ line of the animal at an embryonic stage can be used to examine the effect of increased expression of DNA encoding TAT. Such animals can be used as tester animals for reagents thought to confer protection from, for example, pathological conditions associated with its overexpression. In accordance with this facet of the invention, an animal is treated with the reagent and a reduced incidence of the pathological condition, compared to untreated animals bearing the transgene, would indicate a potential therapeutic intervention for the pathological condition.

Alternatively, non-human homologues of TAT can be used to construct a TAT “knock out” animal which has a defective or altered gene encoding TAT as a result of homologous recombination between the endogenous gene encoding TAT and altered genomic DNA encoding TAT introduced into an embryonic stem cell of the animal. For example, cDNA encoding TAT can be used to clone genomic DNA encoding TAT in accordance with established techniques. A portion of the genomic DNA encoding TAT can be deleted or replaced with another gene, such as a gene encoding a selectable marker which can be used to monitor integration. Typically, several kilobases of unaltered flanking DNA (both at the 5′ and 3′ ends) are included in the vector [see e.g., Thomas and Capecchi, Cell, 51:503 (1987) for a description of homologous recombination vectors]. The vector is introduced into an embryonic stem cell line (e.g., by electroporation) and cells in which the introduced DNA has homologously recombined with the endogenous DNA are selected [see e.g., Li et al., Cell. 69:915 (1992)]. The selected cells are then injected into a blastocyst of an animal (e.g., a mouse or rat) to form aggregation chimeras [see e.g., Bradley, in Teratocarcinomas and Embryonic Stem Cells: A Practical Approach, E. J. Robertson, ed. (IRL, Oxford, 1987), pp. 113-152]. A chimeric embryo can then be implanted into a suitable pseudopregnant female foster animal and the embryo brought to term to create a “knock out” animal. Progeny harboring the homologously recombined DNA in their germ cells can be identified by standard techniques and used to breed animals in which all cells of the animal contain the homologously recombined DNA. Knockout animals can be characterized for instance, for their ability to defend against certain pathological conditions and for their development of pathological conditions due to absence of the TAT polypeptide.

Nucleic acid encoding the TAT polypeptides may also be used in gene therapy. In gene therapy applications, genes are introduced into cells in order to achieve in vivo synthesis of a therapeutically effective genetic product, for example for replacement of a defective gene. “Gene therapy” includes both conventional gene therapy where a lasting effect is achieved by a single treatment, and the administration of gene therapeutic agents, which involves the one time or repeated administration of a therapeutically effective DNA or mRNA. Antisense RNAs and DNAs can be used as therapeutic agents for blocking the expression of certain genes in vivo. It has already been shown that short antisense oligonucleotides can be imported into cells where they act as inhibitors, despite their low intracellular concentrations caused by their restricted uptake by the cell membrane. (Zamecnik et al., Proc. Natl. Acad. Sci. USA 83:4143-4146 [1986]). The oligonucleotides can be modified to enhance their uptake, e.g. by substituting their negatively charged phosphodiester groups by uncharged groups.

There are a variety of techniques available for introducing nucleic acids into viable cells. The techniques vary depending upon whether the nucleic acid is transferred into cultured cells in vitro, or in vivo in the cells of the intended host. Techniques suitable for the transfer of nucleic acid into mammalian cells in vitro include the use of liposomes, electroporation, microinjection, cell fusion, DEAE-dextran, the calcium phosphate precipitation method, etc. The currently preferred in vivo gene transfer techniques include transfection with viral (typically retroviral) vectors and viral coat protein-liposome mediated transfection (Dzau et al., Trends in Biotechnology 11, 205-210 [1993]). In some situations it is desirable to provide the nucleic acid source with an agent that targets the target cells, such as an antibody specific for a cell surface membrane protein or the target cell, a ligand for a receptor on the target cell, etc. Where liposomes are employed, proteins which bind to a cell surface membrane protein associated with endocytosis may be used for targeting and/or to facilitate uptake, e.g. capsid proteins or fragments thereof tropic for a particular cell type, antibodies for proteins which undergo internalization in cycling, proteins that target intracellular localization and enhance intracellular half-life. The technique of receptor-mediated endocytosis is described, for example, by Wu et al., J. Biol. Chem. 262, 4429-4432 (1987); and Wagner et al., Proc. Natl. Acad. Sci. USA 87, 3410-3414 (1990). For review of gene marking and gene therapy protocols see Anderson et al., Science 256, 808-813 (1992).

The nucleic acid molecules encoding the TAT polypeptides or fragments thereof described herein are useful for chromosome identification In this regard, there exists an ongoing need to identify new chromosome markers, since relatively few chromosome marking reagents, based upon actual sequence data are presently available. Each TAT nucleic acid molecule of the present invention can be used as a chromosome marker.

The TAT polypeptides and nucleic acid molecules of the present invention may also be used diagnostically for tissue typing, wherein the TAT polypeptides of the present invention may be differentially expressed in one tissue as compared to another, preferably in a diseased tissue as compared to a normal tissue of the same tissue type. TAT nucleic acid molecules will find use for generating probes for PCR, Northern analysis, Southern analysis and Western analysis.

This invention encompasses methods of screening compounds to identify those that mimic the TAT polypeptide (agonists) or prevent the effect of the TAT polypeptide (antagonists). Screening assays for antagonist drug candidates are designed to identify compounds that bind or complex with the TAT polypeptides encoded by the genes identified herein, or otherwise interfere with the interaction of the encoded polypeptides with other cellular proteins, including e.g., inhibiting the expression of TAT polypeptide from cells. Such screening assays will include assays amenable to high-throughput screening of chemical libraries, making them particularly suitable for identifying small molecule drug candidates.

The assays can be performed in a variety of formats, including protein-protein binding assays, biochemical screening assays, immunoassays, and cell-based assays, which are well characterized in the art.

All assays for antagonists are common in that they call for contacting the drug candidate with a TAT polypeptide encoded by a nucleic acid identified herein under conditions and for a time sufficient to allow these two components to interact.

In binding assays, the interaction is binding and the complex formed can be isolated or detected in the reaction mixture. In a particular embodiment, the TAT polypeptide encoded by the gene identified herein or the drug candidate is immobilized on a solid phase, e.g., on a microtiter plate, by covalent or non-covalent attachments. Non-covalent attachment generally is accomplished by coating the solid surface with a solution of the TAT polypeptide and drying. Alternatively, an immobilized antibody, e.g., a monoclonal antibody, specific for the TAT polypeptide to be immobilized can be used to anchor it to a solid surface. The assay is performed by adding the non-immobilized component, which may be labeled by a detectable label, to the immobilized component, e.g., the coated surface containing the anchored component. When the reaction is complete, the non-reacted components are removed, e.g., by washing, and complexes anchored on the solid surface are detected. When the originally non-immobilized component carries a detectable label, the detection of label immobilized on the surface indicates that complexing occurred. Where the originally non-immobilized component does not carry a label, complexing can be detected, for example, by using a labeled antibody specifically binding the immobilized complex.

If the candidate compound interacts with but does not bind to a particular TAT polypeptide encoded by a gene identified herein, its interaction with that polypeptide can be assayed by methods well known for detecting protein-protein interactions. Such assays include traditional approaches, such as, e.g., cross-linking, co-immunoprecipitation, and co-purification through gradients or chromatographic columns. In addition, protein-protein interactions can be monitored by using a yeast-based genetic system described by Fields and co-workers (Fields and Song, Nature (London), 340:245-246 (1989); Chien et al., Proc. Natl. Acad. Sci. USA, 88:9578-9582 (1991)) as disclosed by Chevray and Nathans, Proc. Natl. Acad. Sci. USA, 89: 5789-5793 (1991). Many transcriptional activators, such as yeast GAL4, consist of two physically discrete modular domains, one acting as the DNA-binding domain, the other one functioning as the transcription-activation domain. The yeast expression system described in the foregoing publications (generally referred to as the “two-hybrid system”) takes advantage of this property, and employs two hybrid proteins, one in which the target protein is fused to the DNA-binding domain of GAL4, and another, in which candidate activating proteins are fused to the activation domain. The expression of a GAL1-lacZ reporter gene under control of a GAL4-activated promoter depends on reconstitution of GAL4 activity via protein-protein interaction. Colonies containing interacting polypeptides are detected with a chromogenic substrate for β-galactosidase. A complete kit (MATCHMAKER™) for identifying protein-protein interactions between two specific proteins using the two-hybrid technique is commercially available from Clontech. This system can also be extended to map protein domains involved in specific protein interactions as well as to pinpoint amino acid residues that are crucial for these interactions.

Compounds that interfere with the interaction of a gene encoding a TAT polypeptide identified herein and other intra- or extracellular components can be tested as follows: usually a reaction mixture is prepared containing the product of the gene and the intra- or extracellular component under conditions and for a time allowing for the interaction and binding of the two products. To test the ability of a candidate compound to inhibit binding, the reaction is run in the absence and in the presence of the test compound. In addition, a placebo may be added to a third reaction mixture, to serve as positive control. The binding (complex formation) between the test compound and the intra- or extracellular component present in the mixture is monitored as described hereinabove. The formation of a complex in the control reaction(s) but not in the reaction mixture containing the test compound indicates that the test compound interferes with the interaction of the test compound and its reaction partner.

To assay for antagonists, the TAT polypeptide may be added to a cell along with the compound to be screened for a particular activity and the ability of the compound to inhibit the activity of interest in the presence of the TAT polypeptide indicates that the compound is an antagonist to the TAT polypeptide. Alternatively, antagonists may be detected by combining the TAT polypeptide and a potential antagonist with membrane-bound TAT polypeptide receptors or recombinant receptors under appropriate conditions for a competitive inhibition assay. The TAT polypeptide can be labeled, such as by radioactivity, such that the number of TAT polypeptide molecules bound to the receptor can be used to determine the effectiveness of the potential antagonist. The gene encoding the receptor can be identified by numerous methods known to those of skill in the art, for example, ligand panning and FACS sorting. Coligan et al., Current Protocols in Immun., 1(2): Chapter 5 (1991). Preferably, expression cloning is employed wherein polyadenylated RNA is prepared from a cell responsive to the TAT polypeptide and a cDNA library created from this RNA is divided into pools and used to transfect COS cells or other cells that are not responsive to the TAT polypeptide. Transfected cells that are grown on glass slides are exposed to labeled TAT polypeptide. The TAT polypeptide can be labeled by a variety of means including iodination or inclusion of a recognition site for a site-specific protein kinase. Following fixation and incubation, the slides are subjected to autoradiographic analysis. Positive pools are identified and sub-pools are prepared and re-transfected using an interactive sub-pooling and re-screening process, eventually yielding a single clone that encodes the putative receptor.

As an alternative approach for receptor identification, labeled TAT polypeptide can be photoaffinity-linked with cell membrane or extract preparations that express the receptor molecule. Cross-linked material is resolved by PAGE and exposed to X-ray film. The labeled complex containing the receptor can be excised, resolved into peptide fragments, and subjected to protein micro-sequencing. The amino acid sequence obtained from micro-sequencing would be used to design a set of degenerate oligonucleotide probes to screen a cDNA library to identify the gene encoding the putative receptor.

In another assay for antagonists, mammalian cells or a membrane preparation expressing the receptor would be incubated with labeled TAT polypeptide in the presence of the candidate compound. The ability of the compound to enhance or block this interaction could then be measured.

More specific examples of potential antagonists include an oligonucleotide that binds to the fusions of immunoglobulin with TAT polypeptide, and, in particular, antibodies including, without limitation, poly- and monoclonal antibodies and antibody fragments, single-chain antibodies, anti-idiotypic antibodies, and chimeric or humanized versions of such antibodies or fragments, as well as human antibodies and antibody fragments. Alternatively, a potential antagonist may be a closely related protein, for example, a mutated form of the TAT polypeptide that recognizes the receptor but imparts no effect, thereby competitively inhibiting the action of the TAT polypeptide.

Another potential TAT polypeptide antagonist is an antisense RNA or DNA construct prepared using antisense technology, where, e.g., an antisense RNA or DNA molecule acts to block directly the translation of mRNA by hybridizing to targeted mRNA and preventing protein translation. Antisense technology can be used to control gene expression through triple-helix formation or antisense DNA or RNA, both of which methods are based on binding of a polynucleotide to DNA or RNA. For example, the 5′ coding portion of the polynucleotide sequence, which encodes the mature TAT polypeptides herein, is used to design an antisense RNA oligonucleotide of from about 10 to 40 base pairs in length. A DNA oligonucleotide is designed to be complementary to a region of the gene involved in transcription (triple helix—see Lee et al., Nucl. Acids Res., 6:3073 (1979); Cooney et al., Science, 241: 456 (1988); Dervan et al., Science, 251:1360 (1991)), thereby preventing transcription and the production of the TAT polypeptide. The antisense RNA oligonucleotide hybridizes to the mRNA in vivo and blocks translation of the mRNA molecule into the TAT polypeptide (antisense—Okano, Neurochem., 56:560 (1991); Oligodeoxynucleotides as Antisense Inhibitors of Gene Expression (CRC Press: Boca Raton, Fla., 1988). The oligonucleotides described above can also be delivered to cells such that the antisense RNA or DNA may be expressed in vivo to inhibit production of the TAT polypeptide. When antisense DNA is used, oligodeoxyribonucleotides derived from the translation-initiation site, e.g., between about −10 and +10 positions of the target gene nucleotide sequence, are preferred.

Potential antagonists include small molecules that bind to the active site, the receptor binding site, or growth factor or other relevant binding site of the TAT polypeptide, thereby blocking the normal biological activity of the TAT polypeptide. Examples of small molecules include, but are not limited to, small peptides or peptide-like molecules, preferably soluble peptides, and synthetic non-peptidyl organic or inorganic compounds.

Ribozymes are enzymatic RNA molecules capable of catalyzing the specific cleavage of RNA. Ribozymes act by sequence-specific hybridization to the complementary target RNA, followed by endonucleolytic cleavage. Specific ribozyme cleavage sites within a potential RNA target can be identified by known techniques. For further details see, e.g., Ross Current Biology, 4:469-471 (1994), and PCT publication No. WO 97/33551 (published Sep. 18, 1997).

Nucleic acid molecules in triple-helix formation used to inhibit transcription should be single-stranded and composed of deoxynucleotides. The base composition of these oligonucleotides is designed such that it promotes triple-helix formation via Hoogsteen base-pairing rules, which generally require sizeable stretches of purines or pyrimidines on one strand of a duplex. For further details see, e.g., PCT publication No. WO 97/33551, supra.

These small molecules can be identified by any one or more of the screening assays discussed hereinabove and/or by any other screening techniques well known for those skilled in the art.

Isolated TAT polypeptide-encoding nucleic acid can be used herein for recombinantly producing TAT polypeptide using techniques well known in the art and as described herein. In turn, the produced TAT polypeptides can be employed for generating anti-TAT antibodies using techniques well known in the art and as described herein.

Antibodies specifically binding a TAT polypeptide identified herein, as well as other molecules identified by the screening assays disclosed hereinbefore, can be administered for the treatment of various disorders, including cancer, in the form of pharmaceutical compositions.

If the TAT polypeptide is intracellular and whole antibodies are used as inhibitors, internalizing antibodies are preferred. However, lipofections or liposomes can also be used to deliver the antibody, or an antibody fragment, into cells. Where antibody fragments are used, the smallest inhibitory fragment that specifically binds to the binding domain of the target protein is preferred. For example, based upon the variable-region sequences of an antibody, peptide molecules can be designed that retain the ability to bind the target protein sequence. Such peptides can be synthesized chemically and/or produced by recombinant DNA technology. See, e.g., Marasco et al., Proc. Natl. Acad. Sci. USA, 90: 7889-7893 (1993).

The formulation herein may also contain more than one active compound as necessary for the particular indication being treated, preferably those with complementary activities that do not adversely affect each other. Alternatively, or in addition, the composition may comprise an agent that enhances its function, such as, for example, a cytotoxic agent, cytokine, chemotherapeutic agent, or growth-inhibitory agent. Such molecules are suitably present in combination in amounts that are effective for the purpose intended.

The following examples are offered for illustrative purposes only, and are not intended to limit the scope of the present invention in any way.

All patent and literature references cited in the present specification are hereby incorporated by reference in their entirety.

### EXAMPLES

Commercially available reagents referred to in the examples were used according to manufacturer's instructions unless otherwise indicated. The source of those cells identified in the following examples, and throughout the specification, by ATCC accession numbers is the American Type Culture Collection, Manassas, Va.

### Example 1

### Analysis of Differential TAT Polypeptide Expression by GEPIS

An expressed sequence tag (EST) DNA database (LIFESEQ®, Incyte Pharmaceuticals, Palo Alto, Calif.) was searched and interesting EST sequences were identified by GEPIS. Gene expression profiling in silico (GEPIS) is a bioinformatics tool developed at Genentech, Inc. that characterizes genes of interest for new cancer therapeutic targets. GEPIS takes advantage of large amounts of EST sequence and library information to determine gene expression profiles. GEPIS is capable of determining the expression profile of a gene based upon its proportional correlation with the number of its occurrences in EST databases, and it works by integrating the LIFESEQ® EST relational database and Genentech proprietary information in a stringent and statistically meaningful way. In this example, GEPIS is used to identify and cross-validate novel tumor antigens, although GEPIS can be configured to perform either very specific analyses or broad screening tasks. For the initial screen, GEPIS is used to identify EST sequences from the LIFESEQ® database that correlate to expression in a particular tissue or tissues of interest (often a tumor tissue of interest). Then, GEPIS was employed to generate a complete tissue expression profile for the various sequences of interest. Using this type of screening bioinformatics, various TAT polypeptides (and their encoding nucleic acid molecules) were identified as being significantly overexpressed in a particular type of cancer or certain cancers as compared to other cancers and/or normal non-cancerous tissues. The rating of GEPIS hits is based upon several criteria including, for example, tissue specificity, tumor specificity and expression level in normal essential and/or normal proliferating tissues. The following is a list of molecules whose tissue expression profile as determined by GEPIS evidences significant upregulation of expression in a specific tumor or tumors as compared to other tumor(s) and/or normal tissues and optionally relatively low expression in normal essential and/or normal proliferating tissues.

Under each tissue heading shown below is a list of the cDNA sequences that are detectably overexpressed in tumor tissue of the indicated tissue type as compared to normal non-tumor tissue of the same tissue type. As such, the molecules listed below (and the polypeptides they encode) are excellent nucleic acid (and polypeptide) targets for the diagnosis and therapy of cancer in mammals.

PERIPHERAL NERVOUS SYSTEM

| | | | | | |
|---|---|---|---|---|---|
| DNA324303 | DNA324573 | DNA324681 | DNA325296 | DNA325405 | DNA325407 |
| DNA325408 | DNA325409 | DNA325410 | DNA325449 | DNA325503 | DNA326083 |
| DNA326231 | DNA188229 | DNA327080 | DNA327081 | DNA327082 | |

BRAIN

| | | | | | |
|---|---|---|---|---|---|
| DNA323721 | DNA323722 | DNA323723 | DNA323724 | DNA323726 | DNA323727 |
| DNA323728 | DNA323729 | DNA323731 | DNA323732 | DNA287173 | DNA151148 |
| DNA323740 | DNA323742 | DNA323743 | DNA323744 | DNA323751 | DNA323753 |
| DNA323755 | DNA323757 | DNA323759 | DNA323764 | DNA323765 | DNA323778 |
| DNA323781 | DNA323783 | DNA323785 | DNA323795 | DNA323796 | DNA323797 |
| DNA323805 | DNA323810 | DNA323811 | DNA323812 | DNA323814 | DNA83085 |
| DNA323817 | DNA323821 | DNA273060 | DNA323823 | DNA323824 | DNA256503 |
| DNA323825 | DNA323826 | DNA323828 | DNA323829 | DNA323830 | DNA323833 |
| DNA103214 | DNA323834 | DNA323837 | DNA323838 | DNA323839 | DNA323846 |
| DNA323856 | DNA323859 | DNA323863 | DNA323869 | DNA323871 | DNA323874 |
| DNA323882 | DNA323887 | DNA323888 | DNA323892 | DNA323893 | DNA323897 |
| DNA323898 | DNA323900 | DNA323901 | DNA323902 | DNA323908 | DNA210134 |
| DNA323912 | DNA323918 | DNA323921 | DNA323922 | DNA323923 | DNA323924 |
| DNA323925 | DNA323926 | DNA257916 | DNA323927 | DNA323931 | DNA323936 |
| DNA323937 | DNA323938 | DNA323939 | DNA323940 | DNA323942 | DNA226793 |
| DNA294794 | DNA323943 | DNA323944 | DNA323946 | DNA323947 | DNA323950 |
| DNA323951 | DNA103436 | DNA323953 | DNA323958 | DNA323959 | DNA323961 |
| DNA226619 | DNA323962 | DNA323964 | DNA323969 | DNA323970 | DNA323973 |
| DNA323974 | DNA323975 | DNA323976 | DNA323977 | DNA323979 | DNA323980 |
| DNA323991 | DNA323992 | DNA323994 | DNA323995 | DNA324000 | DNA324001 |
| DNA324002 | DNA324003 | DNA227246 | DNA324004 | DNA324008 | DNA324009 |
| DNA324010 | DNA324011 | DNA324012 | DNA196344 | DNA193882 | DNA324024 |
| DNA324034 | DNA324037 | DNA324042 | DNA324046 | DNA324047 | DNA324048 |
| DNA324050 | DNA324051 | DNA324055 | DNA275195 | DNA324059 | DNA324060 |
| DNA275049 | DNA324063 | DNA324065 | DNA324066 | DNA324067 | DNA324071 |
| DNA324072 | DNA324073 | DNA227165 | DNA324074 | DNA324076 | DNA324077 |
| DNA324078 | DNA324079 | DNA324080 | DNA271243 | DNA324081 | DNA324082 |
| DNA324084 | DNA324088 | DNA324090 | DNA324091 | DNA324092 | DNA324099 |
| DNA324101 | DNA324106 | DNA324109 | DNA324111 | DNA324112 | DNA324121 |
| DNA324122 | DNA324123 | DNA324128 | DNA324129 | DNA227795 | DNA324130 |
| DNA324131 | DNA324132 | DNA324133 | DNA227528 | DNA324134 | DNA150725 |
| DNA324136 | DNA324138 | DNA324139 | DNA324141 | DNA324146 | DNA324152 |
| DNA324153 | DNA324155 | DNA324159 | DNA324160 | DNA324161 | DNA324162 |
| DNA194740 | DNA324166 | DNA324175 | DNA324176 | DNA272127 | DNA324177 |
| DNA324182 | DNA324184 | DNA324186 | DNA324188 | DNA324194 | DNA324197 |
| DNA324198 | DNA324203 | DNA324204 | DNA324207 | DNA324209 | DNA324210 |
| DNA324216 | DNA324218 | DNA324220 | DNA324221 | DNA324222 | DNA324223 |
| DNA324224 | DNA324227 | DNA324228 | DNA194827 | DNA324230 | DNA324231 |
| DNA324233 | DNA324234 | DNA324235 | DNA324237 | DNA324239 | DNA254204 |
| DNA324240 | DNA189697 | DNA324243 | DNA324246 | DNA324251 | DNA324253 |
| DNA150884 | DNA324256 | DNA324258 | DNA324260 | DNA324262 | DNA324264 |
| DNA324269 | DNA324270 | DNA324271 | DNA324274 | DNA324275 | DNA269910 |
| DNA324279 | DNA324285 | DNA324286 | DNA324288 | DNA324290 | DNA270401 |
| DNA226547 | DNA324295 | DNA324296 | DNA324299 | DNA324300 | DNA324304 |
| DNA324305 | DNA324308 | DNA324309 | DNA324310 | DNA324313 | DNA324314 |
| DNA324315 | DNA324316 | DNA324317 | DNA103505 | DNA324318 | DNA324319 |
| DNA324320 | DNA324323 | DNA324327 | DNA324328 | DNA324329 | DNA324330 |
| DNA324331 | DNA324333 | DNA324336 | DNA324338 | DNA324342 | DNA324343 |
| DNA324353 | DNA88547 | DNA324356 | DNA324358 | DNA324359 | DNA324361 |
| DNA324363 | DNA324364 | DNA324365 | DNA324366 | DNA324367 | DNA324368 |
| DNA324369 | DNA324371 | DNA324377 | DNA324387 | DNA324388 | DNA324389 |
| DNA324390 | DNA324397 | DNA324398 | DNA324410 | DNA324411 | DNA324412 |
| DNA324413 | DNA254620 | DNA324415 | DNA324417 | DNA324418 | DNA89239 |
| DNA324420 | DNA225592 | DNA324422 | DNA324428 | DNA324429 | DNA324434 |
| DNA324435 | DNA324437 | DNA324441 | DNA324442 | DNA324443 | DNA324448 |
| DNA324449 | DNA324457 | DNA324465 | DNA324466 | DNA324467 | DNA324472 |
| DNA257511 | DNA324483 | DNA324485 | DNA324486 | DNA225919 | DNA324487 |
| DNA324491 | DNA324495 | DNA324496 | DNA324497 | DNA324498 | DNA324510 |
| DNA324512 | DNA324513 | DNA324516 | DNA324518 | DNA324519 | DNA324521 |
| DNA324524 | DNA324525 | DNA227575 | DNA324526 | DNA225920 | DNA324527 |
| DNA225921 | DNA324528 | DNA324531 | DNA324532 | DNA324533 | DNA324534 |
| DNA324538 | DNA324540 | DNA324541 | DNA324542 | DNA324545 | DNA324546 |
| DNA324548 | DNA324558 | DNA324559 | DNA324564 | DNA324577 | DNA324578 |
| DNA288259 | DNA324590 | DNA324591 | DNA324595 | DNA324596 | DNA324597 |
| DNA324600 | DNA324604 | DNA324605 | DNA324613 | DNA324614 | DNA324615 |
| DNA324616 | DNA324618 | DNA324619 | DNA324620 | DNA324624 | DNA324625 |
| DNA83020 | DNA324626 | DNA103380 | DNA226872 | DNA324632 | DNA324640 |
| DNA324642 | DNA324643 | DNA324645 | DNA324646 | DNA324647 | DNA324649 |
| DNA324651 | DNA324652 | DNA324653 | DNA150679 | DNA324654 | DNA324655 |
| DNA324656 | DNA324657 | DNA324658 | DNA324659 | DNA324660 | DNA324661 |
| DNA324662 | DNA324663 | DNA324664 | DNA324665 | DNA324666 | DNA324667 |
| DNA324668 | DNA324669 | DNA324670 | DNA324671 | DNA324672 | DNA324673 |
| DNA324674 | DNA324675 | DNA324676 | DNA324678 | DNA324681 | DNA324682 |
| DNA324685 | DNA324686 | DNA324691 | DNA324694 | DNA324696 | DNA324697 |
| DNA324698 | DNA324700 | DNA324701 | DNA324702 | DNA324704 | DNA324705 |
| DNA225909 | DNA274206 | DNA324706 | DNA324707 | DNA324710 | DNA324711 |
| DNA324714 | DNA324715 | DNA324716 | DNA270675 | DNA324717 | DNA269593 |
| DNA324718 | DNA324719 | DNA324720 | DNA324721 | DNA272171 | DNA324728 |
| DNA324729 | DNA304680 | DNA324730 | DNA324734 | DNA324736 | DNA324737 |
| DNA227204 | DNA324738 | DNA324740 | DNA287246 | DNA324743 | DNA324745 |
| DNA304716 | DNA324748 | DNA324749 | DNA324750 | DNA324751 | DNA324755 |
| DNA324756 | DNA324757 | DNA324758 | DNA227442 | DNA324766 | DNA324767 |
| DNA324768 | DNA324769 | DNA287227 | DNA324771 | DNA324772 | DNA324773 |
| DNA324774 | DNA272263 | DNA287319 | DNA324777 | DNA324778 | DNA324779 |
| DNA324782 | DNA324784 | DNA324785 | DNA324786 | DNA324787 | DNA271040 |
| DNA324789 | DNA324791 | DNA324792 | DNA324794 | DNA324796 | DNA324797 |
| DNA324798 | DNA324799 | DNA324803 | DNA324804 | DNA324805 | DNA324809 |
| DNA324810 | DNA324812 | DNA324817 | DNA324819 | DNA324820 | DNA324821 |
| DNA324826 | DNA324830 | DNA324836 | DNA324837 | DNA324838 | DNA324840 |
| DNA324841 | DNA324842 | DNA324844 | DNA324853 | DNA324866 | DNA324873 |
| DNA324876 | DNA324877 | DNA324878 | DNA324879 | DNA324884 | DNA324885 |
| DNA324886 | DNA324889 | DNA324890 | DNA324891 | DNA324892 | DNA324894 |
| DNA225631 | DNA274326 | DNA324895 | DNA324896 | DNA324899 | DNA324902 |
| DNA324903 | DNA324906 | DNA324907 | DNA324908 | DNA324916 | DNA324917 |
| DNA324918 | DNA324920 | DNA324922 | DNA275334 | DNA324924 | DNA324925 |
| DNA324929 | DNA273865 | DNA324931 | DNA324932 | DNA304707 | DNA324938 |
| DNA324944 | DNA324945 | DNA324947 | DNA324952 | DNA324953 | DNA324955 |
| DNA324960 | DNA304710 | DNA324962 | DNA324963 | DNA324965 | DNA324966 |
| DNA324968 | DNA324969 | DNA324972 | DNA324973 | DNA324974 | DNA324977 |
| DNA324978 | DNA324979 | DNA324980 | DNA324982 | DNA324984 | DNA272090 |
| DNA324988 | DNA324989 | DNA324990 | DNA324996 | DNA324997 | DNA324998 |
| DNA324999 | DNA325002 | DNA325005 | DNA325006 | DNA325012 | DNA325013 |
| DNA325014 | DNA325015 | DNA325019 | DNA325020 | DNA325024 | DNA325026 |
| DNA325027 | DNA325032 | DNA325033 | DNA325034 | DNA325035 | DNA325037 |
| DNA325040 | DNA325041 | DNA325043 | DNA325044 | DNA325045 | DNA325046 |
| DNA325047 | DNA325050 | DNA325052 | DNA325054 | DNA325062 | DNA325064 |
| DNA325065 | DNA274178 | DNA325069 | DNA83022 | DNA325070 | DNA325071 |
| DNA325072 | DNA325073 | DNA225671 | DNA325075 | DNA325076 | DNA227267 |
| DNA325082 | DNA325083 | DNA325084 | DNA325085 | DNA325088 | DNA325102 |
| DNA325103 | DNA325105 | DNA325106 | DNA325111 | DNA325112 | DNA325116 |
| DNA325117 | DNA325118 | DNA325119 | DNA325126 | DNA325128 | DNA325132 |
| DNA325136 | DNA325137 | DNA325138 | DNA325139 | DNA325140 | DNA325141 |
| DNA325143 | DNA325144 | DNA325145 | DNA325146 | DNA325147 | DNA325148 |
| DNA325150 | DNA325151 | DNA325152 | DNA325153 | DNA325155 | DNA325156 |
| DNA325157 | DNA325160 | DNA325161 | DNA325163 | DNA325164 | DNA325165 |
| DNA325166 | DNA325167 | DNA325168 | DNA325170 | DNA325171 | DNA226345 |
| DNA325173 | DNA325174 | DNA325181 | DNA227491 | DNA254771 | DNA89242 |
| DNA325182 | DNA325184 | DNA325187 | DNA325190 | DNA272655 | DNA275322 |
| DNA325197 | DNA325199 | DNA325200 | DNA272213 | DNA325202 | DNA325203 |
| DNA325204 | DNA257309 | DNA325206 | DNA325209 | DNA325211 | DNA325212 |
| DNA289530 | DNA287271 | DNA325214 | DNA325216 | DNA325217 | DNA325218 |
| DNA325219 | DNA325220 | DNA325221 | DNA325222 | DNA218841 | DNA325223 |
| DNA325226 | DNA325229 | DNA88350 | DNA325235 | DNA325236 | DNA325237 |
| DNA325240 | DNA325243 | DNA325246 | DNA325247 | DNA325249 | DNA325250 |
| DNA325252 | DNA325253 | DNA325257 | DNA325258 | DNA325261 | DNA325262 |
| DNA325264 | DNA325265 | DNA325266 | DNA325267 | DNA325268 | DNA325269 |
| DNA325270 | DNA325271 | DNA325273 | DNA325274 | DNA325275 | DNA325276 |
| DNA325278 | DNA325279 | DNA325283 | DNA325288 | DNA325290 | DNA325292 |
| DNA325293 | DNA325296 | DNA325301 | DNA325302 | DNA325303 | DNA325304 |
| DNA325307 | DNA325309 | DNA325310 | DNA325312 | DNA325314 | DNA325315 |
| DNA325316 | DNA325318 | DNA325319 | DNA325320 | DNA325322 | DNA325324 |
| DNA193957 | DNA325325 | DNA325326 | DNA325328 | DNA325329 | DNA325331 |
| DNA325333 | DNA325334 | DNA325335 | DNA325336 | DNA325337 | DNA325338 |
| DNA325341 | DNA304459 | DNA325342 | DNA325343 | DNA325344 | DNA325346 |
| DNA325347 | DNA325348 | DNA325349 | DNA325355 | DNA325360 | DNA325361 |
| DNA325362 | DNA325363 | DNA325364 | DNA325365 | DNA325369 | DNA325372 |
| DNA325375 | DNA325381 | DNA325384 | DNA325385 | DNA325393 | DNA325395 |
| DNA269952 | DNA325396 | DNA325397 | DNA325400 | DNA325402 | DNA325403 |
| DNA325404 | DNA325405 | DNA325407 | DNA325408 | DNA325409 | DNA325410 |
| DNA325413 | DNA325414 | DNA325415 | DNA325417 | DNA325418 | DNA325423 |
| DNA325425 | DNA325426 | DNA325430 | DNA325434 | DNA97285 | DNA325446 |
| DNA325451 | DNA325452 | DNA325453 | DNA325456 | DNA325457 | DNA150974 |
| DNA325458 | DNA287417 | DNA227088 | DNA325462 | DNA325464 | DNA325465 |
| DNA325466 | DNA325469 | DNA287254 | DNA325471 | DNA325474 | DNA325476 |
| DNA325477 | DNA325479 | DNA325480 | DNA325481 | DNA325482 | DNA325483 |
| DNA325484 | DNA325489 | DNA325491 | DNA325492 | DNA325493 | DNA325495 |
| DNA325496 | DNA325497 | DNA325498 | DNA269803 | DNA325500 | DNA325501 |
| DNA325503 | DNA325505 | DNA270721 | DNA189687 | DNA325506 | DNA325511 |
| DNA325512 | DNA325513 | DNA103474 | DNA325514 | DNA325516 | DNA325517 |
| DNA325518 | DNA325519 | DNA325520 | DNA325521 | DNA325522 | DNA325523 |
| DNA88176 | DNA325529 | DNA325530 | DNA325534 | DNA325535 | DNA325539 |
| DNA325540 | DNA325541 | DNA325544 | DNA325545 | DNA325546 | DNA325547 |
| DNA325549 | DNA225752 | DNA325551 | DNA325553 | DNA325554 | DNA325557 |
| DNA325561 | DNA325563 | DNA325566 | DNA325568 | DNA325571 | DNA325572 |
| DNA325573 | DNA325574 | DNA325575 | DNA325579 | DNA325580 | DNA325583 |
| DNA325585 | DNA325586 | DNA325587 | DNA88114 | DNA325592 | DNA325593 |
| DNA325596 | DNA325597 | DNA325600 | DNA325601 | DNA225632 | DNA83180 |
| DNA325603 | DNA325608 | DNA325618 | DNA150997 | DNA325625 | DNA325631 |
| DNA325636 | DNA325638 | DNA325639 | DNA325642 | DNA325643 | DNA325649 |
| DNA325650 | DNA325651 | DNA325652 | DNA325653 | DNA325654 | DNA325655 |
| DNA325656 | DNA325657 | DNA325658 | DNA325659 | DNA325660 | DNA325661 |
| DNA325664 | DNA270458 | DNA227092 | DNA325665 | DNA325669 | DNA325670 |
| DNA325673 | DNA325674 | DNA325675 | DNA325676 | DNA325677 | DNA325679 |
| DNA325680 | DNA325681 | DNA325683 | DNA325684 | DNA325687 | DNA325688 |
| DNA325689 | DNA325690 | DNA325691 | DNA325695 | DNA325698 | DNA325702 |
| DNA325706 | DNA79101 | DNA325709 | DNA325711 | DNA325712 | DNA325717 |
| DNA325720 | DNA325721 | DNA325723 | DNA325724 | DNA325731 | DNA226014 |
| DNA325733 | DNA325736 | DNA325739 | DNA325747 | DNA325750 | DNA325752 |
| DNA325755 | DNA325758 | DNA325761 | DNA325762 | DNA325763 | DNA325766 |
| DNA325768 | DNA325773 | DNA325775 | DNA325776 | DNA325782 | DNA325786 |
| DNA325787 | DNA302016 | DNA325789 | DNA325793 | DNA325794 | DNA325796 |
| DNA325797 | DNA325802 | DNA325806 | DNA325807 | DNA325808 | DNA325809 |
| DNA226853 | DNA325811 | DNA325812 | DNA325814 | DNA325818 | DNA325819 |
| DNA270254 | DNA281436 | DNA325837 | DNA325838 | DNA325840 | DNA325843 |
| DNA325844 | DNA325850 | DNA325851 | DNA325852 | DNA325855 | DNA325856 |
| DNA325858 | DNA325859 | DNA325870 | DNA325875 | DNA325878 | DNA325885 |
| DNA325895 | DNA325902 | DNA225649 | DNA325913 | DNA325915 | DNA325918 |
| DNA325919 | DNA325922 | DNA325924 | DNA325928 | DNA325932 | DNA325935 |
| DNA325938 | DNA325942 | DNA325943 | DNA325946 | DNA325947 | DNA325949 |
| DNA325950 | DNA325951 | DNA325956 | DNA325960 | DNA325974 | DNA325975 |
| DNA325976 | DNA325977 | DNA325980 | DNA325981 | DNA325985 | DNA325986 |
| DNA325991 | DNA325992 | DNA325994 | DNA325995 | DNA325996 | DNA326002 |
| DNA326003 | DNA326005 | DNA326006 | DNA326007 | DNA326010 | DNA326011 |
| DNA226646 | DNA326022 | DNA287331 | DNA326024 | DNA326025 | DNA326026 |
| DNA326028 | DNA326029 | DNA326030 | DNA326032 | DNA326034 | DNA326038 |
| DNA326039 | DNA326040 | DNA326041 | DNA326042 | DNA326046 | DNA326047 |
| DNA326049 | DNA326052 | DNA326053 | DNA326057 | DNA326061 | DNA326062 |
| DNA326064 | DNA326066 | DNA326068 | DNA275181 | DNA326069 | DNA326071 |
| DNA326075 | DNA326076 | DNA326078 | DNA326079 | DNA326080 | DNA326085 |
| DNA326086 | DNA326087 | DNA326091 | DNA273839 | DNA256844 | DNA326092 |
| DNA326093 | DNA256886 | DNA326095 | DNA254781 | DNA326096 | DNA326097 |
| DNA326098 | DNA326099 | DNA326100 | DNA326102 | DNA326103 | DNA326109 |
| DNA326110 | DNA326111 | DNA326112 | DNA326113 | DNA326114 | DNA326115 |
| DNA326116 | DNA326117 | DNA326120 | DNA326121 | DNA326122 | DNA326123 |
| DNA326124 | DNA326125 | DNA326128 | DNA326129 | DNA326130 | DNA326132 |
| DNA326133 | DNA326136 | DNA326139 | DNA326140 | DNA326141 | DNA326144 |
| DNA326145 | DNA326146 | DNA326147 | DNA326149 | DNA326154 | DNA326156 |
| DNA326157 | DNA326158 | DNA254532 | DNA326161 | DNA326162 | DNA326163 |
| DNA326168 | DNA271171 | DNA326170 | DNA326171 | DNA326174 | DNA287355 |
| DNA326177 | DNA326178 | DNA326182 | DNA326185 | DNA326186 | DNA326188 |
| DNA326189 | DNA326190 | DNA326195 | DNA326196 | DNA326197 | DNA326198 |
| DNA326200 | DNA326201 | DNA326202 | DNA326204 | DNA88261 | DNA326205 |
| DNA326206 | DNA326207 | DNA326208 | DNA326209 | DNA326211 | DNA326213 |
| DNA326214 | DNA326218 | DNA326219 | DNA326221 | DNA326222 | DNA326226 |
| DNA326228 | DNA326232 | DNA326233 | DNA326234 | DNA326238 | DNA326241 |
| DNA326242 | DNA326248 | DNA326250 | DNA326251 | DNA326252 | DNA326253 |
| DNA326254 | DNA326257 | DNA326258 | DNA326260 | DNA326264 | DNA326266 |
| DNA97300 | DNA326267 | DNA326268 | DNA326269 | DNA326270 | DNA326271 |
| DNA326273 | DNA297388 | DNA326274 | DNA326276 | DNA326277 | DNA326278 |
| DNA326283 | DNA254198 | DNA326288 | DNA326289 | DNA326290 | DNA326291 |
| DNA326292 | DNA326294 | DNA326295 | DNA326296 | DNA255414 | DNA326298 |
| DNA326299 | DNA326300 | DNA326303 | DNA326307 | DNA326308 | DNA326311 |
| DNA326312 | DN326318 | DNA326319 | DNA326320 | DNA326321 | DNA326322 |
| DNA326323 | DNA66475 | DNA270979 | DNA326328 | DNA326329 | DNA326330 |
| DNA272889 | DNA326331 | DNA326332 | DNA326333 | DNA226389 | DNA326335 |
| DNA326336 | DNA326337 | DNA326340 | DNA326342 | DNA326343 | DNA326345 |
| DNA326346 | DNA88378 | DNA326347 | DNA326350 | DNA257428 | DNA326353 |
| DNA326354 | DNA326356 | DNA326359 | DNA326362 | DNA196642 | DNA270901 |
| DNA326363 | DNA326366 | DNA326367 | DNA326368 | DNA254791 | DNA287425 |
| DNA326372 | DNA326375 | DNA326376 | DNA326378 | DNA326379 | DNA287291 |
| DNA326381 | DNA326382 | DNA326383 | DNA326384 | DNA326386 | DNA326387 |
| DNA150457 | DNA326389 | DNA227055 | DNA326392 | DNA326394 | DNA326396 |
| DNA326397 | DNA326399 | DNA326401 | DNA326403 | DNA88430 | DNA326406 |
| DNA326411 | DNA326412 | DNA326413 | DNA129504 | DNA326415 | DNA326416 |
| DNA326417 | DNA326418 | DNA326419 | DNA326425 | DNA326426 | DNA326427 |
| DNA326428 | DNA326429 | DNA326430 | DNA326431 | DNA326434 | DNA326438 |
| DNA273694 | DNA326439 | DNA326449 | DNA326450 | DNA326451 | DNA326452 |
| DNA326453 | DNA326454 | DNA326457 | DNA326461 | DNA326462 | DNA326465 |
| DNA326470 | DNA326471 | DNA326478 | DNA326481 | DNA326482 | DNA326483 |
| DNA326484 | DNA326485 | DNA326487 | DNA326489 | DNA326490 | DNA326491 |
| DNA326492 | DNA326493 | DNA274101 | DNA326494 | DNA326495 | DNA326496 |
| DNA326499 | DNA326502 | DNA326505 | DNA326506 | DNA326509 | DNA326510 |
| DNA326511 | DNA326514 | DNA287636 | DNA326515 | DNA326516 | DNA326518 |
| DNA326519 | DNA326520 | DNA326521 | DNA326522 | DNA326523 | DNA326528 |
| DNA326529 | DNA326530 | DNA326531 | DNA326532 | DNA326533 | DNA326534 |
| DNA326535 | DNA326536 | DNA326537 | DNA326538 | DNA326540 | DNA274761 |
| DNA272421 | DNA326542 | DNA326546 | DNA326547 | DNA326548 | DNA326550 |
| DNA326552 | DNA326555 | DNA326557 | DNA326559 | DNA227280 | DNA326561 |
| DNA326563 | DNA326569 | DNA326570 | DNA326571 | DNA326572 | DNA326575 |
| DNA218271 | DNA326577 | DNA326578 | DNA326579 | DNA103320 | DNA326584 |
| DNA326585 | DNA274034 | DNA326586 | DNA326587 | DNA326588 | DNA326589 |
| DNA326590 | DNA326591 | DNA326592 | DNA326595 | DNA326596 | DNA326597 |
| DNA326598 | DNA326599 | DNA326600 | DNA326601 | DNA326602 | DNA326603 |
| DNA269630 | DNA326604 | DNA326605 | DNA326609 | DNA326610 | DNA287240 |
| DNA326618 | DNA326622 | DNA326623 | DNA326624 | DNA326625 | DNA227249 |
| DNA326626 | DNA326628 | DNA326633 | DNA326634 | DNA326638 | DNA326641 |
| DNA326642 | DNA326644 | DNA326645 | DNA326646 | DNA326647 | DNA256836 |
| DNA326648 | DNA326650 | DNA326653 | DNA326654 | DNA326656 | DNA326657 |
| DNA326658 | DNA326659 | DNA326662 | DNA326663 | DNA326664 | DNA272347 |
| DNA326669 | DNA326670 | DNA256840 | DNA326671 | DNA326672 | DNA326673 |
| DNA326674 | DNA326677 | DNA326679 | DNA273600 | DNA326680 | DNA326682 |
| DNA326684 | DNA326685 | DNA326686 | DNA326687 | DNA326688 | DNA326689 |
| DNA326691 | DNA326692 | DNA151808 | DNA326696 | DNA326698 | DNA326699 |
| DNA326700 | DNA326702 | DNA326705 | DNA326706 | DNA326710 | DNA326711 |
| DNA326713 | DNA88084 | DNA256533 | DNA251057 | DNA326715 | DNA326716 |
| DNA326717 | DNA326718 | DNA326721 | DNA326722 | DNA326723 | DNA326726 |
| DNA326727 | DNA326729 | DNA326730 | DNA326731 | DNA326734 | DNA326735 |
| DNA326736 | DNA326737 | DNA326739 | DNA273066 | DNA326742 | DNA326743 |
| DNA103239 | DNA326744 | DNA326745 | DNA326746 | DNA326747 | DNA326748 |
| DNA326749 | DNA269481 | DNA326751 | DNA326752 | DNA326754 | DNA326756 |
| DNA326758 | DNA326760 | DNA326761 | DNA273346 | DNA326763 | DNA326765 |
| DNA326766 | DNA272062 | DNA326768 | DNA326769 | DNA326770 | DNA326771 |
| DNA297288 | DNA304662 | DNA326772 | DNA326774 | DNA287270 | DNA326780 |
| DNA326781 | DNA326783 | DNA326785 | DNA287261 | DNA326789 | DNA83170 |
| DNA326796 | DNA326798 | DNA326805 | DNA326806 | DNA150767 | DNA326812 |
| DNA326813 | DNA326817 | DNA326818 | DNA326819 | DNA326820 | DNA326821 |
| DNA226758 | DNA194701 | DNA326823 | DNA326824 | DNA326828 | DNA326829 |
| DNA326831 | DNA326833 | DNA326835 | DNA227472 | DNA326836 | DNA103525 |
| DNA326840 | DNA326841 | DNA273320 | DNA326842 | DNA88569 | DNA326843 |
| DNA326848 | DNA326849 | DNA326852 | DNA326853 | DNA326856 | DNA326857 |
| DNA326861 | DNA326862 | DNA326863 | DNA304670 | DNA326864 | DNA326866 |
| DNA103486 | DNA326869 | DNA326878 | DNA326879 | DNA326884 | DNA326886 |
| DNA326887 | DNA326888 | DNA254572 | DNA326889 | DNA254994 | DNA326891 |
| DNA326894 | DNA326896 | DNA36897 | DNA326901 | DNA226409 | DNA326908 |
| DNA326911 | DNA326912 | DNA326913 | DNA326914 | DNA326916 | DNA255046 |
| DNA225954 | DNA326921 | DNA326922 | DNA326928 | DNA326929 | DNA326930 |
| DNA257549 | DNA304835 | DNA326935 | DNA326940 | DNA269830 | DNA326945 |
| DNA326946 | DNA326948 | DNA254141 | DNA151882 | DNA326949 | DNA326950 |
| DNA326951 | DNA326952 | DNA326953 | DNA326956 | DNA326958 | DNA188740 |
| DNA326959 | DNA290259 | DNA304719 | DNA326963 | DNA326964 | DNA326965 |
| DNA254240 | DNA326970 | DNA326972 | DNA326973 | DNA326974 | DNA326976 |
| DNA326977 | DNA326981 | DNA219225 | DNA270954 | DNA326983 | DNA326985 |
| DNA326988 | DNA326989 | DNA326990 | DNA326991 | DNA326992 | DNA326993 |
| DNA256070 | DNA327000 | DNA327002 | DNA327003 | DNA327004 | DNA327005 |
| DNA269793 | DNA327011 | DNA227689 | DNA274829 | DNA327022 | DNA327023 |
| DNA327024 | DNA327025 | DNA327028 | DNA327030 | DNA327034 | DNA327035 |
| DNA327036 | DNA327042 | DNA271580 | DNA327043 | DNA273992 | DNA327045 |
| DNA327046 | DNA327047 | DNA327051 | DNA327054 | DNA225721 | DNA327058 |
| DNA327059 | DNA327060 | DNA327061 | DNA327062 | DNA327067 | DNA327068 |
| DNA327075 | DNA327076 | DNA327077 | DNA327078 | DNA327085 | DNA76504 |
| DNA327093 | DNA273487 | DNA327098 | DNA327099 | DNA254783 | DNA227917 |
| DNA327112 | DNA327113 | DNA327115 | DNA327116 | DNA227013 | DNA225800 |
| DNA327118 | DNA225655 | DNA327119 | DNA327120 | DNA327126 | DNA327127 |

HEAD AND NECK

| | | | | | |
|---|---|---|---|---|---|
| DNA323805 | DNA323843 | DNA323861 | DNA323883 | DNA323899 | DNA323907 |
| DNA323908 | DNA323909 | DNA323986 | DNA324001 | DNA324039 | DNA270154 |
| DNA324139 | DNA324202 | DNA324258 | DNA324263 | DNA324325 | DNA324338 |
| DNA324393 | DNA272605 | DNA324425 | DNA324480 | DNA324588 | DNA324651 |
| DNA324721 | DNA324751 | DNA324784 | DNA324812 | DNA324830 | DNA227924 |
| DNA324874 | DNA324884 | DNA131588 | DNA89242 | DNA325196 | DNA325303 |
| DNA325352 | DNA325377 | DNA325503 | DNA189687 | DNA325526 | DNA325573 |
| DNA150978 | DNA325624 | DNA79313 | DNA325655 | DNA325656 | DNA325657 |
| DNA325658 | DNA325661 | DNA227094 | DNA254777 | DNA325799 | DNA325801 |
| DNA226853 | DNA325832 | DNA274058 | DNA325857 | DNA325917 | DNA325941 |
| DNA325953 | DNA325968 | DNA325989 | DNA325991 | DNA326015 | DNA326048 |
| DNA326076 | DNA326119 | DNA326135 | DNA326159 | DNA326172 | DNA287355 |
| DNA326316 | DNA326324 | DNA326329 | DNA326331 | DNA326332 | DNA88457 |
| DNA88281 | DNA226011 | DNA326738 | DNA273517 | DNA326839 | DNA326873 |
| DNA326884 | DNA326958 | DNA327038 | DNA327078 | | |

PLACENTA

| | | | | | |
|---|---|---|---|---|---|
| DNA323721 | DNA323723 | DNA323728 | DNA323729 | DNA323734 | DNA287173 |
| DNA323736 | DNA227821 | DNA323738 | DNA323739 | DNA273712 | DNA323741 |
| DNA323747 | DNA323750 | DNA323753 | DNA323756 | DNA323763 | DNA323765 |
| DNA323766 | DNA323773 | DNA323776 | DNA323777 | DNA323778 | DNA323781 |
| DNA323782 | DNA323783 | DNA323784 | DNA196349 | DNA323789 | DNA323791 |
| DNA323792 | DNA323793 | DNA323794 | DNA323800 | DNA323804 | DNA227213 |
| DNA323809 | DNA323811 | DNA189315 | DNA323817 | DNA323819 | DNA323820 |
| DNA323822 | DNA274745 | DNA273060 | DNA272024 | DNA323829 | DNA323831 |
| DNA323832 | DNA323833 | DNA304686 | DNA323834 | DNA323835 | DNA323839 |
| DNA323840 | DNA323841 | DNA323842 | DNA323847 | DNA323856 | DNA323857 |
| DNA323858 | DNA323859 | DNA226260 | DNA323862 | DNA323863 | DNA323867 |
| DNA323868 | DNA323869 | DNA323870 | DNA271003 | DNA323871 | DNA323872 |
| DNA323874 | DNA323875 | DNA323876 | DNA323880 | DNA323882 | DNA323887 |
| DNA323888 | DNA323891 | DNA323892 | DNA323896 | DNA323900 | DNA227529 |
| DNA323902 | DNA323905 | DNA323906 | DNA227577 | DNA323914 | DNA323915 |
| DNA323916 | DNA323920 | DNA323925 | DNA323927 | DNA226125 | DNA323936 |
| DNA323940 | DNA323941 | DNA323944 | DNA323947 | DNA323952 | DNA323954 |
| DNA323959 | DNA323963 | DNA323964 | DNA323966 | DNA323971 | DNA323972 |
| DNA323973 | DNA323974 | DNA323980 | DNA323981 | DNA323996 | DNA323999 |
| DNA324004 | DNA324009 | DNA324014 | DNA324018 | DNA324026 | DNA324030 |
| DNA324031 | DNA324032 | DNA324035 | DNA324037 | DNA324038 | DNA324042 |
| DNA324043 | DNA324044 | DNA324047 | DNA324048 | DNA324049 | DNA324054 |
| DNA275195 | DNA324060 | DNA324063 | DNA324067 | DNA324068 | DNA324070 |
| DNA324072 | DNA324073 | DNA324089 | DNA324090 | DNA324091 | DNA324092 |
| DNA324093 | DNA324096 | DNA324101 | DNA275066 | DNA324106 | DNA324109 |
| DNA324110 | DNA324111 | DNA324112 | DNA324115 | DNA324119 | DNA227795 |
| DNA287167 | DNA324130 | DNA324133 | DNA324134 | DNA150725 | DNA324140 |
| DNA324141 | DNA324142 | DNA324143 | DNA324144 | DNA324150 | DNA324151 |
| DNA324152 | DNA324153 | DNA324154 | DNA324156 | DNA275240 | DNA324169 |
| DNA324170 | DNA324171 | DNA324172 | DNA324175 | DNA324182 | DNA324186 |
| DNA304805 | DNA324189 | DNA324190 | DNA324191 | DNA324193 | DNA324195 |
| DNA324199 | DNA324200 | DNA324201 | DNA324203 | DNA324204 | DNA271608 |
| DNA324206 | DNA324207 | DNA324209 | DNA324210 | DNA324212 | DNA324213 |
| DNA324214 | DNA324215 | DNA324218 | DNA324219 | DNA324224 | DNA324226 |
| DNA324230 | DNA189697 | DNA324244 | DNA324247 | DNA324254 | DNA324258 |
| DNA324260 | DNA324266 | DNA324268 | DNA324269 | DNA324270 | DNA324271 |
| DNA324272 | DNA324274 | DNA324276 | DNA151017 | DNA324277 | DNA324281 |
| DNA324282 | DNA324289 | DNA271187 | DNA269930 | DNA324292 | DNA324293 |
| DNA324294 | DNA226547 | DNA324295 | DNA324298 | DNA324302 | DNA324308 |
| DNA324310 | DNA324311 | DNA324313 | DNA324316 | DNA150562 | DNA254582 |
| DNA324320 | DNA324322 | DNA324326 | DNA324337 | DNA269730 | DNA324338 |
| DNA324339 | DNA324340 | DNA324341 | DNA324343 | DNA324344 | DNA324347 |
| DNA324348 | DNA324350 | DNA324351 | DNA324358 | DNA324360 | DNA324365 |
| DNA324368 | DNA324373 | DNA324375 | DNA324376 | DNA324379 | DNA324380 |
| DNA269858 | DNA324387 | DNA324390 | DNA324396 | DNA324398 | DNA324399 |
| DNA324400 | DNA324402 | DNA324405 | DNA324408 | DNA324409 | DNA324411 |
| DNA324412 | DNA324416 | DNA324417 | DNA324418 | DNA324419 | DNA324423 |
| DNA324430 | DNA324431 | DNA324432 | DNA324434 | DNA324436 | DNA324437 |
| DNA324444 | DNA324445 | DNA324446 | DNA324447 | DNA324448 | DNA270615 |
| DNA324450 | DNA324451 | DNA324452 | DNA324459 | DNA324460 | DNA324461 |
| DNA324463 | DNA324464 | DNA324468 | DNA324469 | DNA324472 | DNA324473 |
| DNA324478 | DNA324479 | DNA257511 | DNA324481 | DNA324483 | DNA324491 |
| DNA324495 | DNA324496 | DNA324501 | DNA324502 | DNA324508 | DNA324510 |
| DNA324512 | DNA324519 | DNA324520 | DNA324521 | DNA324525 | DNA324529 |
| DNA324530 | DNA324531 | DNA324537 | DNA324538 | DNA324539 | DNA324541 |
| DNA324542 | DNA324543 | DNA324544 | DNA324545 | DNA324547 | DNA324549 |
| DNA324550 | DNA324561 | DNA324563 | DNA324564 | DNA324565 | DNA227173 |
| DNA324570 | DNA324571 | DNA324572 | DNA287282 | DNA324576 | DNA324579 |
| DNA324581 | DNA324582 | DNA324583 | DNA324584 | DNA288259 | DNA324586 |
| DNA324590 | DNA324591 | DNA324592 | DNA324593 | DNA324595 | DNA324596 |
| DNA324597 | DNA324598 | DNA324599 | DNA324600 | DNA324601 | DNA324603 |
| DNA324604 | DNA257253 | DNA324611 | DNA324613 | DNA324616 | DNA324617 |
| DNA324618 | DNA324619 | DNA324621 | DNA324622 | DNA324624 | DNA103380 |
| DNA324629 | DNA324630 | DNA324631 | DNA324632 | DNA324633 | DNA324634 |
| DNA324641 | DNA324645 | DNA324646 | DNA324647 | DNA302020 | DNA324650 |
| DNA324677 | DNA324678 | DNA324680 | DNA324682 | DNA226418 | DNA324685 |
| DNA324687 | DNA324688 | DNA324689 | DNA324690 | DNA324693 | DNA227320 |
| DNA324696 | DNA324697 | DNA324707 | DNA324712 | DNA324715 | DNA324716 |
| DNA270675 | DNA324717 | DNA324720 | DNA324722 | DNA324723 | DNA324725 |
| DNA324727 | DNA304680 | DNA324730 | DNA324735 | DNA324736 | DNA324737 |
| DNA324741 | DNA324742 | DNA275630 | DNA324745 | DNA324746 | DNA324751 |
| DNA324752 | DNA324753 | DNA324754 | DNA324756 | DNA324759 | DNA324760 |
| DNA324761 | DNA324763 | DNA324764 | DNA324765 | DNA304661 | DNA324771 |
| DNA324775 | DNA324776 | DNA324777 | DNA324778 | DNA324779 | DNA324780 |
| DNA324781 | DNA324783 | DNA304699 | DNA324785 | DNA271040 | DNA324790 |
| DNA324794 | DNA324796 | DNA324797 | DNA324806 | DNA324811 | DNA324818 |
| DNA324820 | DNA324822 | DNA324824 | DNA324827 | DNA324830 | DNA324832 |
| DNA324833 | DNA324835 | DNA324840 | DNA324841 | DNA324844 | DNA324846 |
| DNA271418 | DNA324849 | DNA324853 | DNA324857 | DNA324859 | DNA324860 |
| DNA324862 | DNA324864 | DNA324866 | DNA324868 | DNA324871 | DNA324872 |
| DNA324889 | DNA324891 | DNA225631 | DNA274326 | DNA324895 | DNA247595 |
| DNA324898 | DNA324900 | DNA324901 | DNA324902 | DNA324909 | DNA324915 |
| DNA324916 | DNA324917 | DNA324920 | DNA275334 | DNA324925 | DNA324926 |
| DNA324928 | DNA324929 | DNA273865 | DNA324934 | DNA324936 | DNA324937 |
| DNA287189 | DNA324939 | DNA324940 | DNA103588 | DNA324950 | DNA324951 |
| DNA324952 | DNA324961 | DNA324965 | DNA324966 | DNA324967 | DNA324968 |
| DNA324975 | DNA324976 | DNA324982 | DNA324986 | DNA272090 | DNA324989 |
| DNA324990 | DNA324991 | DNA324992 | DNA324993 | DNA324994 | DNA324995 |
| DNA270711 | DNA325001 | DNA325002 | DNA325003 | DNA325004 | DNA325006 |
| DNA325008 | DNA325013 | DNA325015 | DNA325021 | DNA325024 | DNA325026 |
| DNA325027 | DNA325028 | DNA325030 | DNA325033 | DNA325034 | DNA325042 |
| DNA325048 | DNA226337 | DNA325051 | DNA325053 | DNA325067 | DNA325078 |
| DNA325079 | DNA325080 | DNA325081 | DNA325087 | DNA325088 | DNA325095 |
| DNA325099 | DNA325101 | DNA325102 | DNA325103 | DNA325104 | DNA325105 |
| DNA325106 | DNA226496 | DNA325111 | DNA325113 | DNA325114 | DNA325116 |
| DNA325117 | DNA325118 | DNA325119 | DNA325123 | DNA131588 | DNA325126 |
| DNA325128 | DNA325129 | DNA325132 | DNA325133 | DNA325136 | DNA325139 |
| DNA325140 | DNA325141 | DNA325144 | DNA325146 | DNA325150 | DNA325152 |
| DNA325153 | DNA325156 | DNA325157 | DNA325162 | DNA325164 | DNA325168 |
| DNA271847 | DNA270991 | DNA325173 | DNA325174 | DNA325175 | DNA325176 |
| DNA325179 | DNA325181 | DNA227491 | DNA325182 | DNA325183 | DNA325184 |
| DNA325185 | DNA325187 | DNA325189 | DNA325190 | DNA325196 | DNA325200 |
| DNA325201 | DNA325202 | DNA254543 | DNA325206 | DNA325209 | DNA325213 |
| DNA325214 | DNA325215 | DNA325219 | DNA325222 | DNA325223 | DNA325225 |
| DNA325228 | DNA325229 | DNA325232 | DNA325244 | DNA325248 | DNA325250 |
| DNA325253 | DNA325259 | DNA325260 | DNA325263 | DNA325265 | DNA325272 |
| DNA325277 | DNA325280 | DNA325289 | DNA325293 | DNA273759 | DNA325294 |
| DNA325301 | DNA325303 | DNA325305 | DNA325308 | DNA325311 | DNA325326 |
| DNA325328 | DNA325329 | DNA325334 | DNA103421 | DNA325343 | DNA325344 |
| DNA325346 | DNA325347 | DNA325353 | DNA325356 | DNA325358 | DNA325359 |
| DNA325360 | DNA325364 | DNA325366 | DNA325370 | DNA325375 | DNA325378 |
| DNA325381 | DNA273521 | DNA325383 | DNA325384 | DNA325389 | DNA325394 |
| DNA325395 | DNA269431 | DNA325405 | DNA325412 | DNA325418 | DNA325424 |
| DNA325430 | DNA325431 | DNA325439 | DNA325441 | DNA325442 | DNA325443 |
| DNA325444 | DNA325445 | DNA325447 | DNA325448 | DNA325451 | DNA325452 |
| DNA325454 | DNA325455 | DNA325456 | DNA270134 | DNA325460 | DNA287417 |
| DNA325463 | DNA325464 | DNA325465 | DNA325468 | DNA325470 | DNA325475 |
| DNA325478 | DNA325479 | DNA325480 | DNA325483 | DNA325486 | DNA325487 |
| DNA325488 | DNA325490 | DNA325494 | DNA325498 | DNA325504 | DNA270721 |
| DNA325506 | DNA325507 | DNA325508 | DNA325513 | DNA325522 | DNA325523 |
| DNA325527 | DNA325529 | DNA325530 | DNA325534 | DNA325535 | DNA325541 |
| DNA325544 | DNA271843 | DNA325556 | DNA325557 | DNA325560 | DNA325567 |
| DNA25570 | DNA325576 | DNA325582 | DNA325584 | DNA325587 | DNA325589 |
| DNA325593 | DNA325595 | DNA325596 | DNA325597 | DNA254624 | DNA325601 |
| DNA225632 | DNA325602 | DNA325610 | DNA325611 | DNA325616 | DNA325618 |
| DNA325621 | DNA325625 | DNA325626 | DNA325627 | DNA325632 | DNA325633 |
| DNA271344 | DNA325640 | DNA325642 | DNA325644 | DNA325645 | DNA325648 |
| DNA227191 | DNA270458 | DNA227092 | DNA325666 | DNA325674 | DNA325680 |
| DNA325681 | DNA304783 | DNA325685 | DNA325686 | DNA325688 | DNA325689 |
| DNA325695 | DNA325699 | DNA325700 | DNA325701 | DNA325704 | DNA325707 |
| DNA325711 | DNA325712 | DNA325720 | DNA325724 | DNA325727 | DNA325728 |
| DNA325729 | DNA304694 | DNA325730 | DNA227474 | DNA325731 | DNA227171 |
| DNA325732 | DNA271492 | DNA325733 | DNA325736 | DNA325737 | DNA325739 |
| DNA325750 | DNA325751 | DNA325752 | DNA325758 | DNA325760 | DNA325762 |
| DNA325763 | DNA325772 | DNA325773 | DNA325775 | DNA325776 | DNA325782 |
| DNA325783 | DNA325784 | DNA325785 | DNA325786 | DNA270677 | DNA325787 |
| DNA302016 | DNA325789 | DNA325792 | DNA325798 | DNA325802 | DNA325805 |
| DNA325806 | DNA325809 | DNA270015 | DNA325810 | DNA325811 | DNA325812 |
| DNA325813 | DNA325814 | DNA325816 | DNA325818 | DNA325820 | DNA304669 |
| DNA281436 | DNA325828 | DNA325829 | DNA325830 | DNA325833 | DNA325834 |
| DNA325837 | DNA325838 | DNA325843 | DNA325844 | DNA325847 | DNA325860 |
| DNA325861 | DNA325862 | DNA325863 | DNA325865 | DNA325866 | DNA325867 |
| DNA325872 | DNA325874 | DNA325876 | DNA325877 | DNA325880 | DNA325881 |
| DNA325882 | DNA325886 | DNA325887 | DNA325888 | DNA325889 | DNA325893 |
| DNA325900 | DNA325903 | DNA325904 | DNA325906 | DNA325908 | DNA325910 |
| DNA325911 | DNA325912 | DNA325913 | DNA325921 | DNA269498 | DNA325922 |
| DNA325925 | DNA325926 | DNA325927 | DNA325933 | DNA325935 | DNA325936 |
| DNA325939 | DNA325941 | DNA325944 | DNA325947 | DNA325948 | DNA325949 |
| DNA325950 | DNA103509 | DNA325959 | DNA325961 | DNA325962 | DNA325963 |
| DNA325965 | DNA325966 | DNA325972 | DNA325973 | DNA325980 | DNA325982 |
| DNA325983 | DNA227559 | DNA325985 | DNA325987 | DNA325988 | DNA325994 |
| DNA325995 | DNA325997 | DNA326001 | DNA326002 | DNA326003 | DNA326010 |
| DNA326016 | DNA326019 | DNA326020 | DNA326021 | DNA326022 | DNA326023 |
| DNA287331 | DNA326028 | DNA326036 | DNA326041 | DNA326044 | DNA326046 |
| DNA326047 | DNA326050 | DNA326051 | DNA326056 | DNA275144 | DNA326058 |
| DNA326063 | DNA326070 | DNA326073 | DNA326075 | DNA326081 | DNA326082 |
| DNA326084 | DNA326088 | DNA273839 | DNA326094 | DNA326097 | DNA326099 |
| DNA326103 | DNA326104 | DNA326105 | DNA326106 | DNA326108 | DNA326116 |
| DNA326117 | DNA326118 | DNA326121 | DNA326122 | DNA326124 | DNA326125 |
| DNA326128 | DNA326129 | DNA326134 | DNA289522 | DNA326136 | DNA326150 |
| DNA326151 | DNA274002 | DNA326152 | DNA326153 | DNA326154 | DNA326155 |
| DNA326156 | DNA326157 | DNA326167 | DNA326168 | DNA271171 | DNA326173 |
| DNA287355 | DNA326176 | DNA326179 | DNA194805 | DNA326181 | DNA326183 |
| DNA326184 | DNA326186 | DNA326188 | DNA326191 | DNA326192 | DNA326195 |
| DNA326196 | DNA326197 | DNA326198 | DNA275408 | DNA326200 | DNA189703 |
| DNA326201 | DNA326203 | DNA304704 | DNA326208 | DNA326210 | DNA326211 |
| DNA326212 | DNA326214 | DNA326217 | DNA326222 | DNA326223 | DNA326224 |
| DNA326225 | DNA326227 | DNA227234 | DNA326233 | DNA326234 | DNA326249 |
| DNA326251 | DNA326252 | DNA326255 | DNA326260 | DNA326261 | DNA326262 |
| DNA97300 | DNA326268 | DNA326272 | DNA326273 | DNA326278 | DNA103401 |
| DNA326285 | DNA326288 | DNA290292 | DNA326289 | DNA326291 | DNA326292 |
| DNA326296 | DNA326305 | DNA326311 | DNA326313 | DNA326314 | DNA326315 |
| DNA326316 | DNA287427 | DNA326322 | DNA326324 | DNA326325 | DNA326326 |
| DNA326330 | DNA326334 | DNA326338 | DNA326339 | DNA326340 | DNA326342 |
| DNA326343 | DNA326344 | DNA326356 | DNA326361 | DNA270901 | DNA326364 |
| DNA97290 | DNA227071 | DNA326369 | DNA287425 | DNA326377 | DNA326381 |
| DNA326384 | DNA326385 | DNA326387 | DNA326388 | DNA227055 | DNA326395 |
| DNA326396 | DNA326397 | DNA150814 | DNA326399 | DNA326406 | DNA326407 |
| DNA326408 | DNA326409 | DNA326410 | DNA326411 | DNA129504 | DNA326415 |
| DNA326421 | DNA326424 | DNA326427 | DNA326430 | DNA326435 | DNA326445 |
| DNA326448 | DNA326449 | DNA326450 | DNA326451 | DNA326452 | DNA326453 |
| DNA326454 | DNA256813 | DNA326457 | DNA326459 | DNA326463 | DNA326464 |
| DNA326466 | DNA326467 | DNA326468 | DNA326469 | DNA326471 | DNA326472 |
| DNA326474 | DNA326477 | DNA326483 | DNA326484 | DNA326485 | DNA326486 |
| DNA326487 | DNA326488 | DNA326489 | DNA326490 | DNA326491 | DNA326495 |
| DNA326496 | DNA326499 | DNA326507 | DNA326508 | DNA326510 | DNA326513 |
| DNA326514 | DNA287636 | DNA326515 | DNA326516 | DNA326518 | DNA326520 |
| DNA326524 | DNA326525 | DNA326529 | DNA326530 | DNA326544 | DNA326548 |
| DNA326549 | DNA326551 | DNA326553 | DNA326557 | DNA326559 | DNA227280 |
| DNA270621 | DNA326563 | DNA326564 | DNA326565 | DNA326567 | DNA326569 |
| DNA326579 | DNA326580 | DNA326585 | DNA287243 | DNA326589 | DNA326593 |
| DNA326594 | DNA326595 | DNA269894 | DNA326596 | DNA326597 | DNA326603 |
| DNA326604 | DNA326606 | DNA326607 | DNA326611 | DNA326612 | DNA326613 |
| DNA326616 | DNA326624 | DNA227249 | DNA326626 | DNA326627 | DNA326631 |
| DNA326632 | DNA326633 | DNA326634 | DNA326636 | DNA326637 | DNA326639 |
| DNA326640 | DNA326641 | DNA326643 | DNA326649 | DNA326651 | DNA326657 |
| DNA273474 | DNA272347 | DNA326669 | DNA326671 | DNA274139 | DNA273600 |
| DNA326680 | DNA326681 | DNA326683 | DNA326686 | DNA326687 | DNA326688 |
| DNA326689 | DNA326690 | DNA326691 | DNA326695 | DNA326698 | DNA326702 |
| DNA326704 | DNA326705 | DNA326706 | DNA326707 | DNA103580 | DNA256533 |
| DNA326714 | DNA274289 | DNA326717 | DNA326719 | DNA326720 | DNA326724 |
| DNA326727 | DNA326728 | DNA274823 | DNA290260 | DNA326733 | DNA326736 |
| DNA273066 | DNA326741 | DNA326742 | DNA326749 | DNA326755 | DNA326756 |
| DNA326757 | DNA326758 | DNA326760 | DNA273346 | DNA254548 | DNA326767 |
| DNA326769 | DNA297288 | DNA326775 | DNA326776 | DNA326777 | DNA326778 |
| DNA287270 | DNA326780 | DNA326781 | DNA326782 | DNA326784 | DNA326786 |
| DNA326787 | DNA326788 | DNA271010 | DNA287290 | DNA326793 | DNA326794 |
| DNA326796 | DNA326797 | DNA326798 | DNA326799 | DNA326804 | DNA326807 |
| DNA326808 | DNA326809 | DNA326812 | DNA326814 | DNA326815 | DNA97298 |
| DNA326819 | DNA326822 | DNA194701 | DNA326827 | DNA326831 | DNA103525 |
| DNA326845 | DNA326847 | DNA326855 | DNA326856 | DNA326858 | DNA326866 |
| DNA103486 | DNA326870 | DNA326871 | DNA326873 | DNA326877 | DNA326879 |
| DNA326880 | DNA326881 | DNA269746 | DNA326883 | DNA326884 | DNA326885 |
| DNA326886 | DNA254572 | DNA274129 | DNA326895 | DNA326899 | DNA326900 |
| DNA326901 | DNA326902 | DNA326915 | DNA226617 | DNA326917 | DNA326920 |
| DNA326921 | DNA326928 | DNA326933 | DNA326934 | DNA326935 | DNA326936 |
| DNA326937 | DNA326938 | DNA326940 | DNA326941 | DNA269830 | DNA326943 |
| DNA326944 | DNA103462 | DNA326946 | DNA326947 | DNA254141 | DNA270697 |
| DNA326952 | DNA326953 | DNA151752 | DNA326956 | DNA326957 | DNA188740 |
| DNA326964 | DNA326965 | DNA254240 | DNA326966 | DNA326967 | DNA326968 |
| DNA326974 | DNA326975 | DNA326976 | DNA326977 | DNA326978 | DNA254165 |
| DNA326980 | DNA326981 | DNA270954 | DNA326983 | DNA326984 | DNA326985 |
| DNA326986 | DNA326988 | DNA326989 | DNA326990 | DNA326992 | DNA326994 |
| DNA326996 | DNA326997 | DNA326999 | DNA327003 | DNA327005 | DNA327015 |
| DNA327018 | DNA327021 | DNA327023 | DNA327025 | DNA327029 | DNA327030 |
| DNA327031 | DNA327032 | DNA327037 | DNA327039 | DNA238039 | DNA273992 |
| DNA327046 | DNA327047 | DNA327048 | DNA327049 | DNA327051 | DNA327054 |
| DNA327058 | DNA327060 | DNA327062 | DNA327063 | DNA327064 | DNA327067 |
| DNA327068 | DNA327069 | DNA327070 | DNA327071 | DNA327073 | DNA327074 |
| DNA327077 | DNA327078 | DNA327079 | DNA254785 | DNA327086 | DNA327087 |
| DNA327088 | DNA327094 | DNA327095 | DNA327096 | DNA327097 | DNA327103 |
| DNA327104 | DNA327105 | DNA327107 | DNA327108 | DNA327109 | DNA327110 |
| DNA254783 | DNA327111 | DNA327114 | DNA327115 | DNA327116 | DNA327117 |
| DNA227013 | DNA230792 | DNA103558 | DNA327122 | DNA327123 | |

PINEAL GLAND

| | | | | | |
|---|---|---|---|---|---|
| DNA287173 | DNA323879 | DNA323924 | DNA273088 | DNA323988 | DNA324002 |
| DNA324042 | DNA324048 | DNA324090 | DNA324091 | DNA324092 | DNA324216 |
| DNA324229 | DNA324246 | DNA324296 | DNA324340 | DNA324341 | DNA324521 |
| DNA324554 | DNA324561 | DNA324575 | DNA324636 | DNA324642 | DNA324731 |
| DNA324737 | DNA227607 | DNA304668 | DNA287319 | DNA324784 | DNA324815 |
| DNA324816 | DNA324872 | DNA324885 | DNA225631 | DNA324905 | DNA324930 |
| DNA226416 | DNA324940 | DNA324943 | DNA325026 | DNA325027 | DNA225671 |
| DNA325208 | DNA325231 | DNA325234 | DNA325296 | DNA325475 | DNA271324 |
| DNA325601 | DNA225632 | DNA325642 | DNA325644 | DNA325786 | DNA302016 |
| DNA325789 | DNA325803 | DNA325804 | DNA325883 | DNA325932 | DNA326099 |
| DNA28755 | DNA326363 | DNA326543 | DNA326672 | DNA326909 | DNA326910 |
| DNA327009 | DNA327023 | DNA327025 | DNA327121 | | |

LYMPH NODE

| | | | | | |
|---|---|---|---|---|---|
| DNA227213 | DNA323858 | DNA323859 | DNA323862 | DNA323863 | DNA323864 |
| DNA323866 | DNA323872 | DNA323887 | DNA323925 | DNA226619 | DNA324056 |
| DNA324091 | DNA324092 | DNA324099 | DNA324100 | DNA324113 | DNA324154 |
| DNA324155 | DNA324193 | DNA324204 | DNA324218 | DNA324417 | DNA324418 |
| DNA324434 | DNA324472 | DNA324495 | DNA324501 | DNA324503 | DNA324504 |
| DNA324505 | DNA324521 | DNA324525 | DNA324551 | DNA324552 | DNA324554 |
| DNA324555 | DNA324556 | DNA324557 | DNA324558 | DNA324574 | DNA324575 |
| DNA324595 | DNA324596 | DNA324613 | DNA324632 | DNA324645 | DNA324682 |
| DNA324690 | DNA304680 | DNA324737 | DNA324756 | DNA324785 | DNA324790 |
| DNA324828 | DNA324829 | DNA324841 | DNA324904 | DNA324905 | DNA324906 |
| DNA324907 | DNA324908 | DNA324981 | DNA324982 | DNA324989 | DNA324991 |
| DNA324992 | DNA325006 | DNA325079 | DNA325111 | DNA325126 | DNA325156 |
| DNA325157 | DNA325179 | DNA287216 | DNA288247 | DNA325231 | DNA325233 |
| DNA325234 | DNA325235 | DNA325236 | DNA325250 | DNA325326 | DNA325346 |
| DNA325347 | DNA325360 | DNA325384 | DNA325389 | DNA325535 | DNA325576 |
| DNA325601 | DNA225632 | DNA325625 | DNA325642 | DNA325683 | DNA325684 |
| DNA325750 | DNA325752 | DNA325758 | DNA325786 | DNA302016 | DNA325789 |
| DNA325913 | DNA151893 | DNA325935 | DNA325954 | DNA325955 | DNA325985 |
| DNA325991 | DNA325994 | DNA326002 | DNA326022 | DNA287331 | DNA326041 |
| DNA326046 | DNA326047 | DNA326075 | DNA326095 | DNA326099 | DNA326121 |
| DNA326146 | DNA97300 | DNA270975 | DNA326373 | DNA326416 | DNA326427 |
| DNA326449 | DNA326457 | DNA326459 | DNA326463 | DNA326633 | DNA326742 |
| DNA326885 | DNA326952 | DNA326974 | DNA327023 | DNA327025 | |

COLON

| | | | | | |
|---|---|---|---|---|---|
| DNA287173 | DNA323865 | DNA323867 | DNA323871 | DNA323947 | DNA323964 |
| DNA324039 | DNA324048 | DNA324090 | DNA324091 | DNA324092 | DNA324111 |
| DNA324112 | DNA227795 | DNA324155 | DNA226547 | DNA324417 | DNA324418 |
| DNA324423 | DNA324437 | DNA324495 | DNA324496 | DNA324501 | DNA324502 |
| DNA324504 | DNA324505 | DNA324521 | DNA324525 | DNA324550 | DNA324552 |
| DNA324556 | DNA324557 | DNA324558 | DNA324575 | DNA324604 | DNA324613 |
| DNA324624 | DNA324697 | DNA324717 | DNA324720 | DNA304680 | DNA324737 |
| DNA324756 | DNA324785 | DNA324790 | DNA324828 | DNA324829 | DNA324865 |
| DNA324904 | DNA324905 | DNA324906 | DNA324907 | DNA324908 | DNA324989 |
| DNA325026 | DNA325027 | DNA325033 | DNA325068 | DNA325104 | DNA325105 |
| DNA325106 | DNA325116 | DNA325128 | DNA325129 | DNA325156 | DNA325157 |
| DNA325182 | DNA325183 | DNA325184 | DNA325231 | DNA325232 | DNA325233 |
| DNA325234 | DNA325235 | DNA325236 | DNA325250 | DNA325326 | DNA325347 |
| DNA325358 | DNA325414 | DNA325418 | DNA189687 | DNA325570 | DNA325601 |
| DNA225632 | DNA325605 | DNA325619 | DNA256072 | DNA325642 | DNA325644 |
| DNA270458 | DNA227092 | DNA325731 | DNA226014 | DNA325786 | DNA302016 |
| DNA325789 | DNA325810 | DNA325811 | DNA325812 | DNA325913 | DNA325914 |
| DNA325941 | DNA325985 | DNA326002 | DNA287331 | DNA326099 | DNA326121 |
| DNA326122 | DNA326124 | DNA326136 | DNA326330 | DNA326396 | DNA326457 |
| DNA326529 | DNA326617 | DNA326633 | DNA326634 | DNA326651 | DNA290260 |
| DNA273517 | DNA326886 | DNA226409 | DNA326958 | DNA327025 | DNA327029 |
| DNA327067 | | | | | |

PANCREAS

| | | | | | |
|---|---|---|---|---|---|
| DNA323732 | DNA287173 | DNA323745 | DNA323778 | DNA323781 | DNA323783 |
| DNA323803 | DNA323806 | DNA323808 | DNA323815 | DNA103253 | DNA304686 |
| DNA323856 | DNA323864 | DNA323866 | DNA323878 | DNA323882 | DNA210134 |
| DNA323920 | DNA323923 | DNA323927 | DNA323951 | DNA226619 | DNA226005 |
| DNA83046 | DNA324017 | DNA324042 | DNA324048 | DNA324073 | DNA324091 |
| DNA324092 | DNA324119 | DNA227795 | DNA227528 | DNA324139 | DNA324155 |
| DNA324193 | DNA324195 | DNA324197 | DNA324216 | DNA324220 | DNA324221 |
| DNA324229 | DNA324317 | DNA324320 | DNA324340 | DNA324341 | DNA324352 |
| DNA324364 | DNA324366 | DNA324367 | DNA324380 | DNA324398 | DNA324412 |
| DNA324417 | DNA324418 | DNA324495 | DNA324501 | DNA324504 | DNA324505 |
| DNA324521 | DNA324536 | DNA324552 | DNA324557 | DNA324558 | DNA288259 |
| DNA324591 | DNA83020 | DNA324636 | DNA324642 | DNA324697 | DNA324702 |
| DNA324715 | DNA324716 | DNA324717 | DNA304680 | DNA324737 | DNA227204 |
| DNA324744 | DNA324756 | DNA324770 | DNA272263 | DNA324784 | DNA324790 |
| DNA324795 | DNA324824 | DNA324828 | DNA324829 | DNA324850 | DNA324858 |
| DNA324880 | DNA324884 | DNA324885 | DNA324891 | DNA225631 | DNA274326 |
| DNA324896 | DNA324904 | DNA324906 | DNA324922 | DNA324930 | DNA324935 |
| DNA304710 | DNA324962 | DNA324963 | DNA324972 | DNA324973 | DNA324977 |
| DNA272090 | DNA83141 | DNA325009 | DNA325027 | DNA325033 | DNA304685 |
| DNA325064 | DNA325079 | DNA325099 | DNA325104 | DNA325105 | DNA325106 |
| DNA325126 | DNA325136 | DNA325146 | DNA325156 | DNA325157 | DNA290319 |
| DNA254771 | DNA89242 | DNA325184 | DNA325185 | DNA325202 | DNA325229 |
| DNA88350 | DNA325233 | DNA325235 | DNA325236 | DNA325247 | DNA325254 |
| DNA325262 | DNA325268 | DNA325296 | DNA325330 | DNA325332 | DNA325335 |
| DNA325336 | DNA287237 | DNA325355 | DNA325360 | DNA325384 | DNA325398 |
| DNA325403 | DNA325405 | DNA325411 | DNA325414 | DNA325418 | DNA325428 |
| DNA97285 | DNA325450 | DNA325453 | DNA325475 | DNA325493 | DNA325506 |
| DNA325532 | DNA325548 | DNA325596 | DNA325601 | DNA225632 | DNA226771 |
| DNA325642 | DNA325655 | DNA325656 | DNA325657 | DNA325658 | DNA325660 |
| DNA325661 | DNA325663 | DNA270458 | DNA227092 | DNA196351 | DNA325680 |
| DNA325740 | DNA325741 | DNA325742 | DNA325743 | DNA325744 | DNA325745 |
| DNA325746 | DNA325750 | DNA325752 | DNA325757 | DNA325758 | DNA325760 |
| DNA325775 | DNA325776 | DNA325786 | DNA325788 | DNA325803 | DNA325804 |
| DNA325826 | DNA325912 | DNA103509 | DNA325952 | DNA325953 | DNA326003 |
| DNA326016 | DNA287331 | DNA326047 | DNA326053 | DNA326055 | DNA326058 |
| DNA150485 | DNA326060 | DNA326072 | DNA326092 | DNA326099 | DNA326110 |
| DNA326129 | DNA326157 | DNA326165 | DNA326166 | DNA287355 | DNA326210 |
| DNA326220 | DNA326233 | DNA326234 | DNA97300 | DNA326288 | DNA326291 |
| DNA326292 | DNA326300 | DNA326328 | DNA326330 | DNA326331 | DNA326333 |
| DNA326352 | DNA326370 | DNA326378 | DNA326397 | DNA88430 | DNA326410 |
| DNA326415 | DNA326416 | DNA326426 | DNA326480 | DNA326481 | DNA326482 |
| DNA256555 | DNA326523 | DNA326563 | DNA326577 | DNA326603 | DNA326604 |
| DNA326615 | DNA326621 | DNA326625 | DNA227249 | DNA326646 | DNA326657 |
| DNA326663 | DNA326664 | DNA326665 | DNA326666 | DNA326667 | DNA272347 |
| DNA326668 | DNA326669 | DNA326671 | DNA274139 | DNA326675 | DNA326680 |
| DNA326692 | DNA326698 | DNA326712 | DNA326717 | DNA304658 | DNA326752 |
| DNA326760 | DNA326762 | DNA273346 | DNA254548 | DNA326769 | DNA326776 |
| DNA326777 | DNA287270 | DNA326790 | DNA326803 | DNA326818 | DNA326829 |
| DNA194807 | DNA103525 | DNA326860 | DNA326879 | DNA226409 | DNA326907 |
| DNA326911 | DNA326912 | DNA326913 | DNA326952 | DNA326955 | DNA304719 |
| DNA327023 | DNA327025 | DNA327042 | DNA273254 | DNA327116 | DNA227013 |
| DNA103558 | DNA327120 | | | | |

PROSTATE

| | | | | | |
|---|---|---|---|---|---|
| DNA287173 | DNA323749 | DNA323774 | DNA323779 | DNA323780 | DNA323806 |
| DNA323820 | DNA304686 | DNA323850 | DNA323864 | DNA323866 | DNA323867 |
| DNA323871 | DNA323877 | DNA323882 | DNA227529 | DNA323925 | DNA323927 |
| DNA323944 | DNA226619 | DNA323964 | DNA323980 | DNA323982 | DNA271986 |
| DNA324001 | DNA324004 | DNA83046 | DNA324023 | DNA227504 | DNA324027 |
| DNA324042 | DNA324048 | DNA324057 | DNA324058 | DNA324073 | DNA324090 |
| DNA324091 | DNA324092 | DNA324101 | DNA324111 | DNA324112 | DNA324115 |
| DNA324116 | DNA324117 | DNA227795 | DNA324154 | DNA324155 | DNA324178 |
| DNA324203 | DNA324219 | DNA324230 | DNA324260 | DNA324293 | DNA226547 |
| DNA324301 | DNA227307 | DNA324335 | DNA324340 | DNA324341 | DNA324354 |
| DNA324406 | DNA324412 | DNA324417 | DNA324418 | DNA324437 | DNA324458 |
| DNA324472 | DNA324494 | DNA324502 | DNA324503 | DNA324504 | DNA324505 |
| DNA324521 | DNA324525 | DNA324541 | DNA324550 | DNA324551 | DNA324552 |
| DNA324554 | DNA324555 | DNA324556 | DNA324557 | DNA324558 | DNA324561 |
| DNA324566 | DNA324567 | DNA324575 | DNA324576 | DNA288259 | DNA324587 |
| DNA324595 | DNA324596 | DNA254147 | DNA324604 | DNA324605 | DNA324613 |
| DNA324624 | DNA324631 | DNA324632 | DNA324636 | DNA324645 | DNA324682 |
| DNA324690 | DNA324712 | DNA324715 | DNA324716 | DNA324720 | DNA324722 |
| DNA304680 | DNA324737 | DNA324785 | DNA324793 | DNA324796 | DNA324797 |
| DNA150772 | DNA324825 | DNA324828 | DNA324829 | DNA324830 | DNA324841 |
| DNA324844 | DNA324847 | DNA324856 | DNA324866 | DNA225631 | DNA193955 |
| DNA324904 | DNA324905 | DNA324906 | DNA227929 | DNA324910 | DNA324911 |
| DNA324912 | DNA324926 | DNA103588 | DNA324961 | DNA325006 | DNA325015 |
| DNA325026 | DNA325027 | DNA325079 | DNA325086 | DNA151010 | DNA325098 |
| DNA325105 | DNA325106 | DNA325115 | DNA325116 | DNA131588 | DNA325126 |
| DNA325127 | DNA272050 | DNA325129 | DNA325131 | DNA325156 | DNA325157 |
| DNA325179 | DNA325182 | DNA325184 | DNA325187 | DNA325202 | DNA325210 |
| DNA325231 | DNA325232 | DNA325233 | DNA325234 | DNA325235 | DNA325236 |
| DNA325250 | DNA325303 | DNA325326 | DNA227172 | DNA325335 | DNA103421 |
| DNA325347 | DNA226217 | DNA325349 | DNA325351 | DNA325360 | DNA325398 |
| DNA325414 | DNA325432 | DNA325472 | DNA325475 | DNA325535 | DNA325558 |
| DNA325570 | DNA325576 | DNA325601 | DNA225632 | DNA325618 | DNA325642 |
| DNA325644 | DNA325645 | DNA325655 | DNA270458 | DNA325667 | DNA325668 |
| DNA325680 | DNA325681 | DNA325723 | DNA325731 | DNA325749 | DNA325750 |
| DNA325752 | DNA325786 | DNA302016 | DNA325789 | DNA325801 | DNA325806 |
| DNA325811 | DNA325812 | DNA325814 | DNA325815 | DNA281436 | DNA325836 |
| DNA325841 | DNA325844 | DNA325853 | DNA325854 | DNA325906 | DNA325907 |
| DNA325908 | DNA325913 | DNA325927 | DNA325984 | DNA325985 | DNA325994 |
| DNA325998 | DNA326002 | DNA234442 | DNA287331 | DNA326041 | DNA326046 |
| DNA326054 | DNA326075 | DNA326099 | DNA326122 | DNA326124 | DNA326129 |
| DNA326136 | DNA326155 | DNA287355 | DNA326194 | DNA326201 | DNA326233 |
| DNA326234 | DNA326245 | DNA326254 | DNA97300 | DNA326291 | DNA326292 |
| DNA326302 | DNA326332 | DNA326340 | DNA97290 | DNA326370 | DNA326456 |
| DNA326457 | DNA326459 | DNA326481 | DNA326482 | DNA326529 | DNA326599 |
| DNA326608 | DNA326634 | DNA326645 | DNA326686 | DNA326687 | DNA326688 |
| DNA326692 | DNA103580 | DNA150784 | DNA270931 | DNA254548 | DNA326839 |
| DNA326884 | DNA326893 | DNA326921 | DNA326974 | DNA327005 | DNA327012 |
| DNA327023 | DNA327025 | DNA327039 | DNA273254 | DNA327067 | |

LIVER

| | | | | | |
|---|---|---|---|---|---|
| DNA323720 | DNA323733 | DNA287173 | DNA323758 | DNA323767 | DNA323778 |
| DNA323783 | DNA188748 | DNA323808 | DNA227213 | DNA323810 | DNA323817 |
| DNA323820 | DNA273060 | DNA323852 | DNA269708 | DNA323864 | DNA323865 |
| DNA323866 | DNA323867 | DNA323871 | DNA323894 | DNA323895 | DNA274759 |
| DNA323913 | DNA323917 | DNA323922 | DNA323927 | DNA323934 | DNA323936 |
| DNA323948 | DNA323960 | DNA226619 | DNA323964 | DNA323968 | DNA323971 |
| DNA323972 | DNA323973 | DNA323974 | DNA323983 | DNA323984 | DNA323989 |
| DNA324019 | DNA254346 | DNA324039 | DNA324042 | DNA82328 | DNA324048 |
| DNA324053 | DNA275195 | DNA324063 | DNA324069 | DNA324090 | DNA324091 |
| DNA324092 | DNA324095 | DNA271060 | DNA324111 | DNA324112 | DNA324118 |
| DNA324124 | DNA324125 | DNA227795 | DNA287167 | DNA227528 | DNA324134 |
| DNA324139 | DNA324141 | DNA324154 | DNA324155 | DNA324158 | DNA324174 |
| DNA324181 | DNA324195 | DNA324199 | DNA324200 | DNA324201 | DNA324203 |
| DNA324204 | DNA324205 | DNA271608 | DNA324208 | DNA324217 | DNA324229 |
| DNA324238 | DNA324245 | DNA324258 | DNA324283 | DNA252367 | DNA324293 |
| DNA226547 | DNA324312 | DNA324313 | DNA324320 | DNA324321 | DNA324326 |
| DNA324340 | DNA324341 | DNA324349 | DNA324351 | DNA324355 | DNA324370 |
| DNA324378 | DNA324386 | DNA324414 | DNA324417 | DNA324418 | DNA324437 |
| DNA324439 | DNA324464 | DNA324474 | DNA324476 | DNA324481 | DNA225919 |
| DNA324492 | DNA324495 | DNA324496 | DNA324501 | DNA324502 | DNA324503 |
| DNA324504 | DNA324505 | DNA225584 | DNA324521 | DNA324525 | DNA324541 |
| DNA324550 | DNA324551 | DNA324552 | DNA324554 | DNA324555 | DNA324556 |
| DNA324557 | DNA324558 | DNA324561 | DNA324569 | DNA324575 | DNA324576 |
| DNA324580 | DNA324581 | DNA324582 | DNA288259 | DNA324591 | DNA324596 |
| DNA324600 | DNA324606 | DNA324613 | DNA324618 | DNA103380 | DNA324632 |
| DNA324635 | DNA324636 | DNA324638 | DNA324648 | DNA324685 | DNA324687 |
| DNA324690 | DNA324695 | DNA324700 | DNA324702 | DNA324713 | DNA324717 |
| DNA324722 | DNA324724 | DNA324726 | DNA324727 | DNA304680 | DNA324732 |
| DNA324733 | DNA324736 | DNA324737 | DNA275630 | DNA324744 | DNA304716 |
| DNA324751 | DNA324753 | DNA324756 | DNA287319 | DNA324780 | DNA324781 |
| DNA324783 | DNA304699 | DNA324785 | DNA324790 | DNA324802 | DNA324824 |
| DNA324828 | DNA324829 | DNA324844 | DNA324866 | DNA324881 | DNA225631 |
| DNA274326 | DNA324902 | DNA324904 | DNA324905 | DNA324906 | DNA324907 |
| DNA324908 | DNA324915 | DNA324916 | DNA324917 | DNA324922 | DNA324927 |
| DNA324931 | DNA103588 | DNA324944 | DNA324950 | DNA324951 | DNA324961 |
| DNA304710 | DNA324962 | DNA324963 | DNA324968 | DNA324971 | DNA324974 |
| DNA324977 | DNA272090 | DNA324989 | DNA324991 | DNA324992 | DNA325009 |
| DNA325013 | DNA325018 | DNA325026 | DNA325027 | DNA325033 | DNA325036 |
| DNA325039 | DNA325078 | DNA325079 | DNA325080 | DNA325081 | DNA32509 |
| DNA325091 | DNA325092 | DNA325104 | DNA325105 | DNA325106 | DNA325113 |
| DNA325117 | DNA325118 | DNA325119 | DNA131588 | DNA325126 | DNA325135 |
| DNA325152 | DNA325153 | DNA325156 | DNA325157 | DNA325162 | DNA325177 |
| DNA325179 | DNA89242 | DNA325182 | DNA325184 | DNA325185 | DNA325188 |
| DNA325194 | DNA325231 | DNA325232 | DNA325233 | DNA325234 | DNA325235 |
| DNA325236 | DNA325250 | DNA325280 | DNA325281 | DNA325282 | DNA325287 |
| DNA325296 | DNA325326 | DNA325332 | DNA325334 | DNA325335 | DNA325339 |
| DNA325340 | DNA103506 | DNA325343 | DNA325344 | DNA325347 | DNA325352 |
| DNA325358 | DNA325360 | DNA325368 | DNA325388 | DNA255696 | DNA325403 |
| DNA325408 | DNA325409 | DNA325410 | DNA325411 | DNA325414 | DNA325418 |
| DNA97285 | DNA325456 | DNA226080 | DNA325471 | DNA325473 | DNA325475 |
| DNA325485 | DNA270721 | DNA325506 | DNA325524 | DNA325535 | DNA325536 |
| DNA325537 | DNA325564 | DNA325565 | DNA325570 | DNA325571 | DNA325590 |
| DNA325591 | DNA325596 | DNA325599 | DNA325601 | DNA225632 | DNA226771 |
| DNA325625 | DNA325633 | DNA325637 | DNA325642 | DNA325644 | DNA325645 |
| DNA270458 | DNA227092 | DNA325674 | DNA290294 | DNA325678 | DNA325680 |
| DNA325681 | DNA325686 | DNA325692 | DNA325693 | DNA325694 | DNA325722 |
| DNA325731 | DNA325732 | DNA325750 | DNA325752 | DNA325756 | DNA325758 |
| DNA325778 | DNA325779 | DNA325780 | DNA325786 | DNA302016 | DNA325789 |
| DNA325803 | DNA325804 | DNA325809 | DNA325811 | DNA325812 | DNA325814 |
| DNA325823 | DNA325837 | DNA325838 | DNA325842 | DNA325845 | DNA325849 |
| DNA325853 | DNA325854 | DNA325863 | DNA325868 | DNA325869 | DNA325871 |
| DNA325882 | DNA325887 | DNA325896 | DNA325906 | DNA325908 | DNA325912 |
| DNA325929 | DNA325931 | DNA325935 | DNA226324 | DNA325949 | DNA325971 |
| DNA325978 | DNA325979 | DNA325985 | DNA325999 | DNA326002 | DNA326003 |
| DNA326006 | DNA326017 | DNA287331 | DNA326069 | DNA326099 | DNA326101 |
| DNA326121 | DNA326122 | DNA326124 | DNA326127 | DNA326129 | DNA326136 |
| DNA326156 | DNA326164 | DNA287355 | DNA326193 | DNA326196 | DNA189703 |
| DNA326220 | DNA326233 | DNA326234 | DNA326239 | DNA326242 | DNA326246 |
| DNA326247 | DNA326254 | DNA326256 | DNA97300 | DNA326273 | DNA326278 |
| DNA254198 | DNA326289 | DNA326291 | DNA326292 | DNA326325 | DNA326330 |
| DNA326334 | DNA326339 | DNA326341 | DNA88378 | DNA326347 | DNA326352 |
| DNA326357 | DNA326370 | DNA326380 | DNA227055 | DNA326406 | DNA274755 |
| DNA326411 | DNA326416 | DNA326423 | DNA326426 | DNA326427 | DNA326430 |
| DNA326434 | DNA326437 | DNA326440 | DNA326449 | DNA326450 | DNA326451 |
| DNA326452 | DNA326453 | DNA326454 | DNA326457 | DNA326476 | DNA326481 |
| DNA326482 | DNA326484 | DNA326485 | DNA326489 | DNA326497 | DNA326498 |
| DNA326539 | DNA326548 | DNA326563 | DNA326579 | DNA326580 | DNA326586 |
| DNA326625 | DNA227249 | DNA326626 | DNA326633 | DNA326634 | DNA326646 |
| DNA326651 | DNA326671 | DNA326678 | DNA326680 | DNA326698 | DNA326701 |
| DNA326702 | DNA326703 | DNA326705 | DNA326706 | DNA103580 | DNA326713 |
| DNA88084 | DNA326727 | DNA290260 | DNA326736 | DNA326741 | DNA326742 |
| DNA326752 | DNA326756 | DNA326758 | DNA326762 | DNA254548 | DNA326769 |
| DNA304662 | DNA326772 | DNA326776 | DNA326777 | DNA227348 | DNA326819 |
| DNA194701 | DNA326826 | DNA326831 | DNA326832 | DNA326850 | DNA326851 |
| DNA269526 | DNA326867 | DNA326870 | DNA326871 | DNA269746 | DNA326885 |
| DNA326886 | DNA326905 | DNA326923 | DNA326924 | DNA326939 | DNA269830 |
| DNA326947 | DNA326958 | DNA188740 | DNA326964 | DNA326974 | DNA326977 |
| DNA326981 | DNA270954 | DNA326983 | DNA326987 | DNA326992 | DNA327003 |
| DNA327005 | DNA327010 | DNA327013 | DNA327014 | DNA327016 | DNA327023 |
| DNA327025 | DNA327027 | DNA327050 | DNA327052 | DNA327053 | DNA273254 |
| DNA327065 | DNA327067 | DNA327068 | DNA327069 | DNA327091 | DNA227656 |
| DNA327106 | DNA327114 | DNA327116 | DNA227013 | | |

BONE MARROW

| | | | | | |
|---|---|---|---|---|---|
| DNA323735 | DNA323762 | DNA323770 | DNA323771 | DNA323774 | DNA323775 |
| DNA323784 | DNA323804 | DNA272748 | DNA323880 | DNA323903 | DNA323904 |
| DNA323964 | DNA323982 | DNA324015 | DNA324023 | DNA324056 | DNA324057 |
| DNA324076 | DNA324086 | DNA324100 | DNA324139 | DNA324154 | DNA324173 |
| DNA324178 | DNA324200 | DNA324211 | DNA324230 | DNA324242 | DNA324248 |
| DNA324249 | DNA324250 | DNA324260 | DNA88100 | DNA324301 | DNA324364 |
| DNA324381 | DNA324382 | DNA324383 | DNA324420 | DNA324484 | DNA324495 |
| DNA324507 | DNA324551 | DNA324554 | DNA324575 | DNA324605 | DNA324637 |
| DNA324644 | DNA324690 | DNA304680 | DNA324746 | DNA324825 | DNA324848 |
| DNA324854 | DNA324856 | DNA324858 | DNA324905 | DNA324910 | DNA325011 |
| DNA325031 | DNA325086 | DNA151010 | DNA325127 | DNA272050 | DNA325133 |
| DNA325169 | DNA325184 | DNA325231 | DNA325234 | DNA325241 | DNA325242 |
| DNA325299 | DNA287642 | DNA325345 | DNA325351 | DNA325354 | DNA325356 |
| DNA325392 | DNA325399 | DNA325428 | DNA325461 | DNA272413 | DNA325576 |
| DNA325668 | DNA325726 | DNA325733 | DNA325811 | DNA325901 | DNA325953 |
| DNA151831 | DNA325998 | DNA234442 | DNA326035 | DNA326095 | DNA326138 |
| DNA326365 | DNA326373 | DNA326390 | DNA326391 | DNA326416 | DNA326417 |
| DNA326449 | DNA326450 | DNA326451 | DNA326942 | DNA327111 | |

TESTIS

| | | | | | |
|---|---|---|---|---|---|
| DNA287173 | DNA323761 | DNA323770 | DNA323771 | DNA323774 | DNA323775 |
| DNA226262 | DNA323778 | DNA323790 | DNA323804 | DNA323817 | DNA323820 |
| DNA323829 | DNA103214 | DNA304686 | DNA272748 | DNA323844 | DNA323845 |
| DNA323851 | DNA323856 | DNA323858 | DNA323859 | DNA323861 | DNA323864 |
| DNA323865 | DNA323866 | DNA323867 | DNA323869 | DNA323871 | DNA323872 |
| DNA323877 | DNA323880 | DNA323922 | DNA323943 | DNA323947 | DNA323956 |
| DNA323964 | DNA323967 | DNA323968 | DNA323973 | DNA323985 | DNA323993 |
| DNA323998 | DNA324004 | DNA324009 | DNA324015 | DNA324023 | DNA324048 |
| DNA324054 | DNA324058 | DNA324063 | DNA324090 | DNA324091 | DNA324092 |
| DNA324100 | DNA324103 | DNA324111 | DNA324112 | DNA324114 | DNA324117 |
| DNA324118 | DNA227795 | DNA150725 | DNA324147 | DNA324149 | DNA324154 |
| DNA324155 | DNA324164 | DNA324165 | DNA324170 | DNA324173 | DNA324178 |
| DNA324187 | DNA304805 | DNA324196 | DNA324199 | DNA324200 | DNA324201 |
| DNA299899 | DNA324204 | DNA271608 | DNA324207 | DNA324208 | DNA324210 |
| DNA324213 | DNA324214 | DNA324218 | DNA324219 | DNA324229 | DNA324230 |
| DNA324276 | DNA324281 | DNA324282 | DNA324284 | DNA324285 | DNA324291 |
| DNA324293 | DNA226547 | DNA324295 | DNA324301 | DNA324312 | DNA324313 |
| DNA324326 | DNA324357 | DNA324358 | DNA324373 | DNA324381 | DNA324382 |
| DNA324383 | DNA324384 | DNA324385 | DNA324390 | DNA324395 | DNA324398 |
| DNA324403 | DNA324404 | DNA324417 | DNA324418 | DNA324423 | DNA324433 |
| DNA324434 | DNA324436 | DNA324437 | DNA324438 | DNA324455 | DNA324468 |
| DNA324469 | DNA324472 | DNA324478 | DNA324479 | DNA324481 | DNA324483 |
| DNA324490 | DNA324491 | DNA324495 | DNA324496 | DNA324499 | DNA324500 |
| DNA324501 | DNA324502 | DNA324503 | DNA324504 | DNA324505 | DNA324507 |
| DNA324509 | DNA324511 | DNA324512 | DNA324514 | DNA324521 | DNA324522 |
| DNA324525 | DNA324531 | DNA324541 | DNA324549 | DNA324550 | DNA324551 |
| DNA324552 | DNA324554 | DNA324555 | DNA324556 | DNA324557 | DNA324558 |
| DNA324568 | DNA324574 | DNA324575 | DNA324576 | DNA324579 | DNA324583 |
| DNA324584 | DNA324585 | DNA324590 | DNA324591 | DNA324592 | DNA324595 |
| DNA324596 | DNA324597 | DNA324598 | DNA324599 | DNA324600 | DNA324601 |
| DNA324605 | DNA269816 | DNA324612 | DNA324613 | DNA324616 | DNA324622 |
| DNA324624 | DNA324628 | DNA324632 | DNA271931 | DNA324642 | DNA324645 |
| DNA324682 | DNA324683 | DNA324684 | DNA324685 | DNA324687 | DNA324690 |
| DNA324697 | DNA324717 | DNA324720 | DNA304680 | DNA324737 | DNA324742 |
| DNA275630 | DNA324746 | DNA324751 | DNA324785 | DNA324790 | DNA324800 |
| DNA324801 | DNA324803 | DNA150772 | DNA324811 | DNA324828 | DNA324829 |
| DNA324831 | DNA324840 | DNA324841 | DNA324843 | DNA324844 | DNA324845 |
| DNA324846 | DNA324855 | DNA324858 | DNA324866 | DNA324867 | DNA324882 |
| DNA324883 | DNA225631 | DNA324902 | DNA324904 | DNA324905 | DNA324906 |
| DNA324907 | DNA324908 | DNA324909 | DNA324910 | DNA324913 | DNA324914 |
| DNA324915 | DNA324916 | DNA324917 | DNA324926 | DNA324928 | DNA324941 |
| DNA324950 | DNA324951 | DNA324954 | DNA304710 | DNA324962 | DNA324963 |
| DNA324965 | DNA324966 | DNA324967 | DNA324968 | DNA324982 | DNA324989 |
| DNA325002 | DNA325003 | DNA325006 | DNA325007 | DNA226560 | DNA325010 |
| DNA325011 | DNA325025 | DNA325026 | DNA325027 | DNA325028 | DNA325034 |
| DNA325049 | DNA325078 | DNA325079 | DNA325080 | DNA325081 | DNA325086 |
| DNA325095 | DNA325096 | DNA151010 | DNA325097 | DNA325098 | DNA325107 |
| DNA325111 | DNA325116 | DNA325117 | DNA325118 | DNA325119 | DNA325123 |
| DNA325124 | DNA325125 | DNA131588 | DNA325127 | DNA325134 | DNA325141 |
| DNA325146 | DNA325152 | DNA325153 | DNA325154 | DNA325155 | DNA325156 |
| DNA325157 | DNA325158 | DNA325159 | DNA325164 | DNA325169 | DNA325179 |
| DNA325182 | DNA325183 | DNA325184 | DNA325196 | DNA325202 | DNA325206 |
| DNA325222 | DNA325229 | DNA325231 | DNA325232 | DNA325233 | DNA325234 |
| DNA325235 | DNA325236 | DNA325250 | DNA325281 | DNA325282 | DNA325289 |
| DNA325291 | DNA325297 | DNA325298 | DNA325301 | DNA287642 | DNA325326 |
| DNA325339 | DNA325340 | DNA103421 | DNA325345 | DNA325347 | DNA325349 |
| DNA325351 | DNA325357 | DNA325358 | DNA325360 | DNA325376 | DNA325387 |
| DNA325392 | DNA325395 | DNA269952 | DNA255078 | DNA325428 | DNA325430 |
| DNA325433 | DNA325434 | DNA325435 | DNA325436 | DNA325437 | DNA325438 |
| DNA97285 | DNA325439 | DNA325445 | DNA254186 | DNA325523 | DNA325534 |
| DNA325535 | DNA325541 | DNA325549 | DNA272413 | DNA325564 | DNA325565 |
| DNA325570 | DNA257965 | DNA325576 | DNA325589 | DNA325601 | DNA225632 |
| DNA325613 | DNA325615 | DNA325622 | DNA325625 | DNA325629 | DNA325630 |
| DNA325632 | DNA325633 | DNA325635 | DNA325642 | DNA325644 | DNA325645 |
| DNA325668 | DNA325672 | DNA325674 | DNA325680 | DNA325685 | DNA325697 |
| DNA325711 | DNA325720 | DNA325731 | DNA325732 | DNA325736 | DNA325748 |
| DNA325750 | DNA325752 | DNA325753 | DNA325754 | DNA325758 | DNA325762 |
| DNA325782 | DNA325786 | DNA302016 | DNA325789 | DNA325806 | DNA325809 |
| DNA325810 | DNA325811 | DNA325812 | DNA325814 | DNA325821 | DNA304669 |
| DNA325824 | DNA325825 | DNA325827 | DNA325829 | DNA325831 | DNA325837 |
| DNA325838 | DNA325843 | DNA325844 | DNA325848 | DNA325860 | DNA227321 |
| DNA325879 | DNA325882 | DNA325886 | DNA325887 | DNA325888 | DNA325897 |
| DNA325898 | DNA325901 | DNA325905 | DNA325906 | DNA325908 | DNA325913 |
| DNA325922 | DNA325933 | DNA325934 | DNA325935 | DNA325939 | DNA325940 |
| DNA325965 | DNA325969 | DNA325985 | DNA325991 | DNA325994 | DNA325998 |
| DNA326002 | DNA326003 | DNA326009 | DNA234442 | DNA326020 | DNA326021 |
| DNA326022 | DNA287331 | DNA326035 | DNA326041 | DNA326045 | DNA326046 |
| DNA326047 | DNA326070 | DNA326075 | DNA326099 | DNA326128 | DNA326129 |
| DNA326155 | DNA326156 | DNA274180 | DNA326187 | DNA326214 | DNA326228 |
| DNA326233 | DNA326234 | DNA326251 | DNA97300 | DNA304715 | DNA290292 |
| DNA326289 | DNA326291 | DNA326292 | DNA326311 | DNA326364 | DNA326373 |
| DNA326390 | DNA326391 | DNA326397 | DNA326400 | DNA326410 | DNA326426 |
| DNA287234 | DNA326449 | DNA326450 | DNA326451 | DNA326452 | DNA326453 |
| DNA326454 | DNA326457 | DNA326463 | DNA326471 | DNA326557 | DNA326559 |
| DNA326579 | DNA326580 | DNA326603 | DNA326633 | DNA326634 | DNA326642 |
| DNA326651 | DNA326686 | DNA326687 | DNA326688 | DNA326691 | DNA326692 |
| DNA326698 | DNA290260 | DNA304658 | DNA326762 | DNA326769 | DNA326790 |
| DNA326791 | DNA326792 | DNA326796 | DNA326798 | DNA326837 | DNA326854 |
| DNA326858 | DNA326884 | DNA326885 | DNA326886 | DNA326940 | DNA326941 |
| DNA269830 | DNA254240 | DNA326974 | DNA327005 | DNA327019 | DNA327020 |
| DNA327021 | DNA327025 | DNA327026 | DNA327027 | DNA327029 | DNA327039 |
| DNA327044 | DNA327060 | DNA327062 | DNA273254 | DNA327066 | DNA327067 |
| DNA327072 | DNA327077 | DNA327078 | DNA327079 | DNA327083 | DNA327084 |
| DNA327098 | DNA327100 | DNA327114 | | | |

CERVIX

| | | | | | |
|---|---|---|---|---|---|
| DNA324417 | DNA324418 | DNA324557 | DNA324828 | DNA324829 | DNA324904 |
| DNA324905 | DNA324906 | DNA325231 | DNA325234 | | |

NERVOUS

| | | | | | |
|---|---|---|---|---|---|
| DNA287173 | DNA323760 | DNA103253 | DNA323848 | DNA323864 | DNA323865 |
| DNA323866 | DNA323867 | DNA323877 | DNA323878 | DNA323882 | DNA323887 |
| DNA323925 | DNA323966 | DNA324107 | DNA227795 | DNA324135 | DNA227190 |
| DNA324155 | DNA271608 | DNA324219 | DNA324259 | DNA324320 | DNA324351 |
| DNA324364 | DNA270615 | DNA324504 | DNA324505 | DNA324551 | DNA324552 |
| DNA324554 | DNA324555 | DNA324556 | DNA324557 | DNA324558 | DNA324575 |
| DNA324756 | DNA324790 | DNA324828 | DNA324829 | DNA324904 | DNA324905 |
| DNA324906 | DNA324907 | DNA324908 | DNA324982 | DNA325079 | DNA325187 |
| DNA325231 | DNA325232 | DNA325233 | DNA325234 | DNA325235 | DNA325236 |
| DNA325416 | DNA325419 | DNA325432 | DNA325562 | DNA325602 | DNA325607 |
| DNA226028 | DNA325647 | DNA325704 | DNA325759 | DNA287331 | DNA326077 |
| DNA326196 | DNA326198 | DNA326215 | DNA326362 | DNA326459 | DNA326752 |
| DNA326846 | DNA226409 | DNA326956 | DNA326983 | DNA327058 | DNA327099 |

EYE

| | | | | | |
|---|---|---|---|---|---|
| DNA323721 | DNA287173 | DNA323747 | DNA323763 | DNA323769 | DNA226262 |
| DNA323778 | DNA323799 | DNA323807 | DNA227213 | DNA323817 | DNA323818 |
| DNA323820 | DNA323829 | DNA323835 | DNA323839 | DNA323856 | DNA323858 |
| DNA323859 | DNA323864 | DNA323865 | DNA323866 | DNA323869 | DNA323871 |
| DNA323872 | DNA323875 | DNA323887 | DNA323891 | DNA323892 | DNA323906 |
| DNA323914 | DNA323923 | DNA323925 | DNA323928 | DNA323932 | DNA323935 |
| DNA323936 | DNA323947 | DNA323964 | DNA323971 | DNA323972 | DNA323973 |
| DNA323974 | DNA323988 | DNA256905 | DNA324004 | DNA324009 | DNA324010 |
| DNA247474 | DNA324022 | DNA324023 | DNA324025 | DNA324028 | DNA324029 |
| DNA324037 | DNA324048 | DNA324049 | DNA103217 | DNA275195 | DNA324059 |
| DNA324060 | DNA324061 | DNA275049 | DNA324062 | DNA273800 | DNA324076 |
| DNA324083 | DNA324085 | DNA324087 | DNA324090 | DNA324091 | DNA324092 |
| DNA324096 | DNA324100 | DNA226428 | DNA275066 | DNA324104 | DNA324106 |
| DNA324108 | DNA324110 | DNA324111 | DNA324112 | DNA324127 | DNA227795 |
| DNA287167 | DNA324155 | DNA324157 | DNA324163 | DNA324164 | DNA324165 |
| DNA324167 | DNA275240 | DNA324170 | DNA324175 | DNA324185 | DNA324186 |
| DNA324193 | DNA324199 | DNA324200 | DNA324201 | DNA324203 | DNA324204 |
| DNA324207 | DNA324209 | DNA324210 | DNA324212 | DNA324213 | DNA324214 |
| DNA324217 | DNA324218 | DNA324219 | DNA324224 | DNA324230 | DNA324280 |
| DNA324281 | DNA324282 | DNA226547 | DNA324295 | DNA324306 | DNA324307 |
| DNA324312 | DNA324313 | DNA324320 | DNA324322 | DNA324329 | DNA324330 |
| DNA324331 | DNA273919 | DNA324332 | DNA324334 | DNA324338 | DNA324344 |
| DNA324345 | DNA324347 | DNA324358 | DNA324359 | DNA324365 | DNA324372 |
| DNA324374 | DNA324390 | DNA324417 | DNA324418 | DNA324423 | DNA324434 |
| DNA324436 | DNA324437 | DNA324448 | DNA324458 | DNA324461 | DNA324463 |
| DNA324470 | DNA324478 | DNA324479 | DNA324481 | DNA324482 | DNA324483 |
| DNA324491 | DNA324495 | DNA324496 | DNA324501 | DNA324504 | DNA324505 |
| DNA324510 | DNA324512 | DNA324519 | DNA324521 | DNA324525 | DNA324535 |
| DNA324541 | DNA324552 | DNA324555 | DNA324556 | DNA324557 | DNA324558 |
| DNA324575 | DNA324584 | DNA324589 | DNA324590 | DNA324591 | DNA324594 |
| DNA324595 | DNA324596 | DNA324597 | DNA324598 | DNA324599 | DNA324600 |
| DNA254147 | DNA324607 | DNA290231 | DNA324608 | DNA324609 | DNA324613 |
| DNA324623 | DNA324624 | DNA324625 | DNA324632 | DNA324645 | DNA324682 |
| DNA324687 | DNA324690 | DNA324697 | DNA324710 | DNA324711 | DNA324717 |
| DNA324718 | DNA324720 | DNA304680 | DNA324737 | DNA270613 | DNA324742 |
| DNA287246 | DNA324745 | DNA304716 | DNA324747 | DNA324751 | DNA324756 |
| DNA324766 | DNA304661 | DNA324777 | DNA324778 | DNA324779 | DNA324785 |
| DNA324788 | DNA324790 | DNA324811 | DNA324828 | DNA324829 | DNA324830 |
| DNA324839 | DNA324841 | DNA324844 | DNA324866 | DNA324902 | DNA324904 |
| DNA324906 | DNA324907 | DNA324908 | DNA324915 | DNA324916 | DNA324917 |
| DNA324942 | DNA103588 | DNA324948 | DNA324949 | DNA324950 | DNA324951 |
| DNA324965 | DNA324966 | DNA324967 | DNA324968 | DNA324982 | DNA324989 |
| DNA325002 | DNA325003 | DNA325005 | DNA325006 | DNA325013 | DNA325015 |
| DNA325024 | DNA325025 | DNA325026 | DNA325027 | DNA325034 | DNA325058 |
| DNA325066 | DNA325078 | DNA325079 | DNA325080 | DNA325081 | DNA325093 |
| DNA325098 | DNA325110 | DNA325111 | DNA325116 | DNA325117 | DNA325118 |
| DNA325119 | DNA325124 | DNA325127 | DNA325128 | DNA325130 | DNA325146 |
| DNA325152 | DNA325153 | DNA325155 | DNA325156 | DNA325157 | DNA325164 |
| DNA325172 | DNA325179 | DNA325182 | DNA325183 | DNA325184 | DNA325190 |
| DNA325191 | DNA325192 | DNA325193 | DNA325196 | DNA325198 | DNA325202 |
| DNA325206 | DNA271722 | DNA325207 | DNA325209 | DNA325222 | DNA325233 |
| DNA325235 | DNA325236 | DNA325247 | DNA325256 | DNA325283 | DNA325289 |
| DNA325293 | DNA325298 | DNA325300 | DNA325301 | DNA325311 | DNA325313 |
| DNA325317 | DNA325321 | DNA325323 | DNA325347 | DNA325351 | DNA325364 |
| DNA325370 | DNA325376 | DNA325378 | DNA325382 | DNA227509 | DNA325389 |
| DNA325390 | DNA325395 | DNA325427 | DNA325430 | DNA97285 | DNA325439 |
| DNA325442 | DNA325445 | DNA325451 | DNA325452 | DNA270134 | DNA325459 |
| DNA272728 | DNA325478 | DNA325479 | DNA325499 | DNA270721 | DNA325506 |
| DNA325523 | DNA325526 | DNA325534 | DNA325535 | DNA325540 | DNA325542 |
| DNA325543 | DNA271843 | DNA325559 | DNA325576 | DNA325577 | DNA325578 |
| DNA325584 | DNA325587 | DNA325593 | DNA325596 | DNA325598 | DNA325601 |
| DNA225632 | DNA325607 | DNA226028 | DNA325612 | DNA325614 | DNA325625 |
| DNA325627 | DNA325628 | DNA325632 | DNA325642 | DNA325647 | DNA325674 |
| DNA290294 | DNA325678 | DNA325680 | DNA325682 | DNA325683 | DNA325684 |
| DNA325685 | DNA25688 | DNA325690 | DNA325695 | DNA325713 | DNA325719 |
| DNA325720 | DNA325731 | DNA325733 | DNA325736 | DNA274361 | DNA325752 |
| DNA325757 | DNA325762 | DNA325769 | DNA325773 | DNA325775 | DNA325776 |
| DNA325782 | DNA325784 | DNA325786 | DNA302016 | DNA325789 | DNA325800 |
| DNA325810 | DNA325811 | DNA325812 | DNA325817 | DNA325818 | DNA304669 |
| DNA281436 | DNA325835 | DNA325837 | DNA325838 | DNA325843 | DNA325844 |
| DNA210180 | DNA325872 | DNA325882 | DNA325889 | DNA325891 | DNA325892 |
| DNA325899 | DNA325906 | DNA325908 | DNA325922 | DNA325924 | DNA325933 |
| DNA325935 | DNA325945 | DNA325964 | DNA325965 | DNA325975 | DNA325978 |
| DNA325979 | DNA325985 | DNA325988 | DNA326000 | DNA326002 | DNA326004 |
| DNA326008 | DNA234442 | DNA326013 | DNA326016 | DNA326020 | DNA326021 |
| DNA326022 | DNA326031 | DNA326033 | DNA255370 | DNA273014 | DNA326037 |
| DNA326047 | DNA326050 | DNA326058 | DNA326061 | DNA326072 | DNA326097 |
| DNA326099 | DNA326104 | DNA326105 | DNA326116 | DNA326121 | DNA326122 |
| DNA326124 | DNA326129 | DNA326133 | DNA326136 | DNA326156 | DNA326167 |
| DNA326175 | DNA326196 | DNA326197 | DNA326198 | DNA326214 | DNA326221 |
| DNA326222 | DNA326229 | DNA326243 | DNA326244 | DNA326251 | DNA326260 |
| DNA326264 | DNA326265 | DNA97300 | DNA297388 | DNA326288 | DNA290292 |
| DNA326289 | DNA326294 | DNA326296 | DNA326316 | DNA326322 | DNA326334 |
| DNA326339 | DNA326343 | DNA326344 | DNA227873 | DNA326348 | DNA326360 |
| DNA97290 | DNA227071 | DNA227764 | DNA326376 | DNA326381 | DNA326393 |
| DNA326394 | DNA326398 | DNA326402 | DNA326405 | DNA326406 | DNA326413 |
| DNA326418 | DNA326420 | DNA326427 | DNA326435 | DNA326436 | DNA326445 |
| DNA326447 | DNA274690 | DNA326449 | DNA326450 | DNA326451 | DNA326452 |
| DNA326453 | DNA326454 | DNA326455 | DNA326458 | DNA326459 | DNA326463 |
| DNA326466 | DNA326467 | DNA326473 | DNA326488 | DNA326520 | DNA326526 |
| DNA326527 | DNA326534 | DNA326559 | DNA326560 | DNA326574 | DNA326576 |
| DNA326579 | DNA326580 | DNA326615 | DNA326617 | DNA326633 | DNA326634 |
| DNA326642 | DNA326663 | DNA326664 | DNA272347 | DNA326669 | DNA326671 |
| DNA326691 | DNA326694 | DNA326697 | DNA326705 | DNA326706 | DNA256533 |
| DNA326717 | DNA326718 | DNA326719 | DNA326720 | DNA326749 | DNA326753 |
| DNA273346 | DNA326769 | DNA287270 | DNA326779 | DNA326780 | DNA326781 |
| DNA326787 | DNA326795 | DNA326796 | DNA326798 | DNA326819 | DNA326830 |
| DNA326858 | DNA254572 | DNA326892 | DNA326894 | DNA326904 | DNA326919 |
| DNA326931 | DNA326932 | DNA326935 | DNA326940 | DNA326941 | DNA269830 |
| DNA326946 | DNA326952 | DNA326956 | DNA326962 | DNA254240 | DNA326974 |
| DNA326983 | DNA327005 | DNA327006 | DNA327007 | DNA327017 | DNA327019 |
| DNA327021 | DNA327023 | DNA327025 | DNA327026 | DNA327027 | DNA327029 |
| DNA327046 | DNA327058 | DNA327060 | DNA327062 | DNA273254 | DNA327067 |
| DNA327070 | DNA37072 | DNA327077 | DNA327078 | DNA327079 | DNA227181 |
| DNA327099 | DNA327114 | DNA103558 | DNA327125 | | |

OVARY

| | | | | | |
|---|---|---|---|---|---|
| DNA287173 | DNA323865 | DNA323867 | DNA324048 | DNA324148 | DNA324295 |
| DNA324340 | DNA324341 | DNA324642 | DNA324694 | DNA324697 | DNA324737 |
| DNA324874 | DNA325601 | DNA225632 | DNA325720 | DNA325786 | DNA287331 |
| DNA326099 | DNA326657 | DNA327025 | | | |

ADIPOSE

| | | |
|---|---|---|
| DNA325952 | DNA325957 | DNA325958 |

WHOLE BLOOD

| | | | | | |
|---|---|---|---|---|---|
| DNA323718 | DNA323719 | DNA323752 | DNA323754 | DNA323788 | DNA83085 |
| DNA323886 | DNA323889 | DNA323890 | DNA323911 | DNA323957 | DNA323980 |
| DNA324002 | DNA324020 | DNA324021 | DNA324033 | DNA324040 | DNA324041 |
| DNA324052 | DNA324240 | DNA324296 | DNA225910 | DNA324317 | DNA324320 |
| DNA324515 | DNA324560 | DNA324562 | DNA324722 | DNA324742 | DNA324784 |
| DNA324861 | DNA324875 | DNA324884 | DNA324885 | DNA324887 | DNA324888 |
| DNA324923 | DNA325016 | DNA325017 | DNA325038 | DNA325055 | DNA325056 |
| DNA325057 | DNA325059 | DNA325060 | DNA325061 | DNA325063 | DNA325177 |
| DNA325255 | DNA88562 | DNA325335 | DNA325360 | DNA325401 | DNA325516 |
| DNA325609 | DNA325623 | DNA325631 | DNA325641 | DNA290294 | DNA325678 |
| DNA226014 | DNA325750 | DNA325758 | DNA325764 | DNA325803 | DNA281436 |
| DNA325829 | DNA226105 | DNA325912 | DNA326089 | DNA326090 | DNA326113 |
| DNA326115 | DNA326160 | DNA326240 | DNA326254 | DNA88378 | DNA88554 |
| DNA326371 | DNA326479 | DNA326655 | DNA326802 | DNA326834 | DNA88239 |
| DNA326906 | DNA326958 | DNA326977 | DNA327052 | DNA327116 | |

THYROID

| | | | | | |
|---|---|---|---|---|---|
| DNA323717 | DNA188748 | DNA323867 | DNA324154 | DNA324216 | DNA324295 |
| DNA324501 | DNA324503 | DNA324550 | DNA324551 | DNA324554 | DNA324565 |
| DNA324697 | DNA324873 | DNA324874 | DNA324905 | DNA325191 | DNA325192 |
| DNA325232 | DNA325234 | DNA325335 | DNA325503 | DNA325720 | DNA325845 |
| DNA326259 | DNA326275 | DNA326862 | DNA326863 | DNA304670 | DNA326864 |

PITUITARY GLAND

| | | | | | |
|---|---|---|---|---|---|
| DNA323717 | DNA323967 | DNA103593 | DNA324100 | DNA324293 | DNA324326 |
| DNA324610 | DNA324720 | DNA324801 | DNA324846 | DNA324874 | DNA325089 |
| DNA325523 | DNA325533 | DNA325589 | DNA325617 | DNA325967 | DNA325970 |
| DNA325680 | DNA325695 | DNA325700 | DNA325702 | DNA325711 | DNA325712 |
| DNA325724 | DNA325733 | DNA325736 | DNA325738 | DNA325752 | DNA325770 |
| DNA325773 | DNA325775 | DNA325776 | DNA325777 | DNA325786 | DNA325805 |
| DNA325810 | DNA325818 | DNA325837 | DNA325838 | DNA325890 | DNA325900 |
| DNA325906 | DNA325908 | DNA325909 | DNA325913 | DNA325920 | DNA269498 |
| DNA325922 | DNA325925 | DNA325935 | DNA325941 | DNA103509 | DNA325965 |
| DNA227559 | DNA325985 | DNA325994 | DNA326002 | DNA326003 | DNA326022 |
| DNA287331 | DNA326027 | DNA326036 | DNA326041 | DNA326046 | DNA326047 |
| DNA326056 | DNA326076 | DNA273839 | DNA326099 | DNA326107 | DNA326116 |
| DNA326118 | DNA326121 | DNA326122 | DNA326124 | DNA326128 | DNA326129 |
| DNA326133 | DNA326136 | DNA326142 | DNA326156 | DNA326168 | DNA326173 |
| DNA287355 | DNA326178 | DNA326196 | DNA326197 | DNA275408 | DNA326251 |
| DNA326254 | DNA97300 | DNA326272 | DNA326273 | DNA326278 | DNA326288 |
| DNA290292 | DNA326296 | DNA326311 | DNA326316 | DNA326324 | DNA326329 |
| DNA326343 | DNA88378 | DNA326354 | DNA326355 | DNA326358 | DNA326362 |
| DNA227071 | DNA326384 | DNA227055 | DNA326396 | DNA326397 | DNA326406 |
| DNA326408 | DNA326415 | DNA326416 | DNA326426 | DNA326449 | DNA326450 |
| DNA326451 | DNA326452 | DNA326453 | DNA326454 | DNA326457 | DNA326463 |
| DNA326475 | DNA326490 | DNA326499 | DNA326525 | DNA326539 | DNA326559 |
| DNA270621 | DNA326562 | DNA326579 | DNA326580 | DNA326595 | DNA326597 |
| DNA326599 | DNA326603 | DNA326651 | DNA272347 | DNA274139 | DNA326680 |
| DNA326691 | DNA326704 | DNA326709 | DNA304658 | DNA326742 | DNA326752 |
| DNA326760 | DNA273346 | DNA254548 | DNA326769 | DNA287270 | DNA326780 |
| DNA326781 | DNA326790 | DNA326796 | DNA326798 | DNA150548 | DNA326803 |
| DNA326819 | DNA326821 | DNA194701 | DNA326825 | DNA326872 | DNA326884 |
| DNA326886 | DNA254572 | DNA326901 | DNA226617 | DNA326921 | DNA326935 |
| DNA326941 | DNA326947 | DNA326949 | DNA326950 | DNA326952 | DNA326956 |
| DNA326963 | DNA326967 | DNA326974 | DNA326981 | DNA219225 | DNA326983 |
| DNA326984 | DNA326985 | DNA326995 | DNA327003 | DNA327023 | DNA327025 |
| DNA227943 | DNA327056 | DNA327057 | DNA327060 | DNA327062 | DNA273254 |
| DNA327068 | DNA327101 | DNA327107 | DNA327110 | DNA327114 | DNA327115 |
| DNA227013 | | | | | |

THYMUS

| | | | | | |
|---|---|---|---|---|---|
| DNA324063 | DNA324197 | DNA324641 | DNA324685 | DNA324926 | DNA325038 |
| DNA325195 | DNA325238 | DNA325405 | DNA325420 | DNA325421 | DNA325422 |
| DNA325506 | DNA325645 | DNA325809 | DNA325930 | DNA326089 | DNA326090 |
| DNA326243 | DNA326554 | DNA326563 | DNA326747 | | |

MUSCLE

| | | | | | |
|---|---|---|---|---|---|
| DNA323725 | DNA323732 | DNA287173 | DNA323736 | DNA323737 | DNA323740 |
| DNA171408 | DNA323746 | DNA323748 | DNA323749 | DNA323753 | DNA323765 |
| DNA323766 | DNA323767 | DNA323768 | DNA323778 | DNA323779 | DNA323780 |
| DNA323782 | DNA323784 | DNA323789 | DNA323792 | DNA323794 | DNA323798 |
| DNA323801 | DNA323802 | DNA323804 | DNA227213 | DNA323810 | DNA323813 |
| DNA323816 | DNA323817 | DNA274487 | DNA323820 | DNA323821 | DNA323826 |
| DNA323827 | DNA323829 | DNA323830 | DNA323833 | DNA103214 | DNA323837 |
| DNA323839 | DNA323852 | DNA323853 | DNA323854 | DNA323855 | DNA323858 |
| DNA323859 | DNA323860 | DNA323862 | DNA323863 | DNA323864 | DNA323865 |
| DNA323866 | DNA323867 | DNA323869 | DNA323870 | DNA323871 | DNA275139 |
| DNA323872 | DNA323874 | DNA323881 | DNA323882 | DNA323885 | DNA323887 |
| DNA227529 | DNA225809 | DNA323914 | DNA323925 | DNA323929 | DNA323930 |
| DNA323933 | DNA323934 | DNA323936 | DNA194600 | DNA323947 | DNA323949 |
| DNA323955 | DNA323964 | DNA323971 | DNA323972 | DNA323973 | DNA323974 |
| DNA323977 | DNA323978 | DNA323981 | DNA323987 | DNA323995 | DNA323997 |
| DNA290234 | DNA324001 | DNA256905 | DNA324004 | DNA324007 | DNA324014 |
| DNA324016 | DNA324039 | DNA324045 | DNA324048 | DNA324049 | DNA324054 |
| DNA275195 | DNA324058 | DNA324059 | DNA324060 | DNA324063 | DNA324064 |
| DNA273800 | DNA324090 | DNA324091 | DNA324092 | DNA324097 | DNA324098 |
| DNA324109 | DNA324111 | DNA324112 | DNA324120 | DNA324126 | DNA227795 |
| DNA324133 | DNA324135 | DNA324137 | DNA324141 | DNA324145 | DNA324154 |
| DNA324155 | DNA255531 | DNA275240 | DNA324168 | DNA324170 | DNA324182 |
| DNA324183 | DNA88051 | DNA324197 | DNA324199 | DNA324200 | DNA324201 |
| DNA324203 | DNA324204 | DNA324207 | DNA324210 | DNA324217 | DNA324230 |
| DNA324232 | DNA189697 | DNA324241 | DNA324243 | DNA324252 | DNA324255 |
| DNA324257 | DNA324260 | DNA324263 | DNA324267 | DNA324269 | DNA324270 |
| DNA324271 | DNA324278 | DNA324282 | DNA324287 | DNA324294 | DNA226547 |
| DNA324295 | DNA324297 | DNA324313 | DNA324318 | DNA324323 | DNA324324 |
| DNA324329 | DNA324330 | DNA324331 | DNA324338 | DNA324340 | DNA324341 |
| DNA324358 | DNA324371 | DNA324390 | DNA324398 | DNA324400 | DNA324414 |
| DNA324417 | DNA324418 | DNA324421 | DNA324423 | DNA324434 | DNA324437 |
| DNA324440 | DNA324454 | DNA324456 | DNA324461 | DNA324462 | DNA324469 |
| DNA324472 | DNA324478 | DNA324479 | DNA324483 | DNA324488 | DNA324493 |
| DNA324495 | DNA324496 | DNA324501 | DNA324502 | DNA324503 | DNA324504 |
| DNA324505 | DNA324510 | DNA324521 | DNA324523 | DNA324525 | DNA324538 |
| DNA324541 | DNA324550 | DNA324551 | DNA324552 | DNA324554 | DNA324556 |
| DNA324557 | DNA324558 | DNA324564 | DNA324575 | DNA324583 | DNA324584 |
| DNA288259 | DNA324590 | DNA324591 | DNA324592 | DNA324595 | DNA324596 |
| DNA324597 | DNA324598 | DNA324599 | DNA324600 | DNA324602 | DNA324604 |
| DNA324608 | DNA324613 | DNA324624 | DNA324626 | DNA324627 | DNA269809 |
| DNA324632 | DNA324633 | DNA324634 | DNA324636 | DNA324645 | DNA271626 |
| DNA324675 | DNA324678 | DNA324682 | DNA324685 | DNA324690 | DNA324696 |
| DNA324697 | DNA274206 | DNA324707 | DNA324708 | DNA324709 | DNA324710 |
| DNA324711 | DNA324715 | DNA324716 | DNA270675 | DNA324717 | DNA324720 |
| DNA324722 | DNA324723 | DNA304680 | DNA324737 | DNA324739 | DNA324744 |
| DNA304460 | DNA324751 | DNA324756 | DNA324763 | DNA324764 | DNA324769 |
| DNA324770 | DNA324780 | DNA324781 | DNA324783 | DNA304699 | DNA324784 |
| DNA324785 | DNA324790 | DNA324791 | DNA290264 | DNA324794 | DNA324811 |
| DNA324813 | DNA324815 | DNA324823 | DNA324827 | DNA324828 | DNA324829 |
| DNA103471 | DNA324834 | DNA324840 | DNA324841 | DNA324844 | DNA324846 |
| DNA324851 | DNA324852 | DNA324866 | DNA324880 | DNA324884 | DNA324893 |
| DNA225631 | DNA274326 | DNA324896 | DNA324897 | DNA324902 | DNA324904 |
| DNA324905 | DNA324906 | DNA324907 | DNA324908 | DNA324915 | DNA324916 |
| DNA324917 | DNA324921 | DNA324926 | DNA324932 | DNA324933 | DNA287189 |
| DNA103588 | DNA324950 | DNA324951 | DNA324952 | DNA324957 | DNA324958 |
| DNA324959 | DNA324965 | DNA324966 | DNA324967 | DNA324968 | DNA324972 |
| DNA324973 | DNA324977 | DNA324982 | DNA324983 | DNA324985 | DNA324989 |
| DNA324990 | DNA324991 | DNA324992 | DNA325002 | DNA325006 | DNA325013 |
| DNA325015 | DNA325021 | DNA325022 | DNA325023 | DNA325024 | DNA325026 |
| DNA325027 | DNA25034 | DNA325039 | DNA325045 | DNA226337 | DNA325062 |
| DNA325077 | DNA325078 | DNA325079 | DNA325080 | DNA325081 | DNA325094 |
| DNA325095 | DNA325100 | DNA325103 | DNA325109 | DNA226496 | DNA325111 |
| DNA325116 | DNA325117 | DNA325118 | DNA325119 | DNA325122 | DNA131588 |
| DNA325152 | DNA325153 | DNA325156 | DNA325157 | DNA325164 | DNA325168 |
| DNA325174 | DNA325178 | DNA325179 | DNA325182 | DNA325183 | DNA325184 |
| DNA287216 | DNA288247 | DNA325187 | DNA325190 | DNA325196 | DNA325200 |
| DNA325202 | DNA325205 | DNA325206 | DNA325210 | DNA325214 | DNA225630 |
| DNA325216 | DNA325222 | DNA325223 | DNA325227 | DNA325231 | DNA325232 |
| DNA325233 | DNA325234 | DNA325235 | DNA325236 | DNA325239 | DNA325245 |
| DNA325247 | DNA325250 | DNA325295 | DNA325296 | DNA325301 | DNA325303 |
| DNA325308 | DNA325326 | DNA325327 | DNA325344 | DNA304488 | DNA325346 |
| DNA325347 | DNA325358 | DNA325360 | DNA325362 | DNA325367 | DNA325371 |
| DNA325373 | DNA144601 | DNA325375 | DNA325380 | DNA325384 | DNA325389 |
| DNA325406 | DNA325407 | DNA325408 | DNA325409 | DNA325410 | DNA325411 |
| DNA325429 | DNA325440 | DNA325451 | DNA325452 | DNA325459 | DNA272728 |
| DNA325463 | DNA325469 | DNA325474 | DNA325478 | DNA325494 | DNA325498 |
| DNA270721 | DNA325515 | DNA325523 | DNA325531 | DNA325534 | DNA325535 |
| DNA325538 | DNA325552 | DNA325555 | DNA325560 | DNA325576 | DNA325577 |
| DNA325580 | DNA325581 | DNA297398 | DNA325582 | DNA325584 | DNA325585 |
| DNA325587 | DNA325588 | DNA325594 | DNA325597 | DNA254624 | DNA325601 |
| DNA225632 | DNA188396 | DNA226028 | DNA325618 | DNA325620 | DNA325625 |
| DNA325627 | DNA325633 | DNA325637 | DNA272379 | DNA325642 | DNA325644 |
| DNA325645 | DNA325646 | DNA325671 | DNA325674 | DNA325680 | DNA227094 |
| DNA325695 | DNA325703 | DNA137231 | DNA325704 | DNA325705 | DNA325706 |
| DNA325708 | DNA79101 | DNA325709 | DNA325710 | DNA325711 | DNA325712 |
| DNA325714 | DNA325715 | DNA325716 | DNA325718 | DNA325720 | DNA325724 |
| DNA325725 | DNA325731 | DNA325733 | DNA325734 | DNA325750 | DNA325752 |
| DNA325758 | DNA325762 | DNA325767 | DNA325768 | DNA325771 | DNA325773 |
| DNA325775 | DNA325776 | DNA325781 | DNA325784 | DNA325786 | DNA302016 |
| DNA325789 | DNA325790 | DNA325791 | DNA325795 | DNA325806 | DNA325808 |
| DNA325809 | DNA325810 | DNA325811 | DNA325812 | DNA325814 | DNA325815 |
| DNA325826 | DNA325830 | DNA325837 | DNA325838 | DNA325843 | DNA325844 |
| DNA325857 | DNA325867 | DNA325873 | DNA325874 | DNA225865 | DNA325879 |
| DNA325882 | DNA325889 | DNA325891 | DNA325906 | DNA325908 | DNA325910 |
| DNA325911 | DNA325912 | DNA325913 | DNA325925 | DNA325933 | DNA151893 |
| DNA325935 | DNA325937 | DNA103509 | DNA325954 | DNA325955 | DNA325965 |
| DNA325966 | DNA325985 | DNA325994 | DNA326002 | DNA255340 | DNA326012 |
| DNA326014 | DNA326018 | DNA326022 | DNA287331 | DNA326027 | DNA326036 |
| DNA326040 | DNA326041 | DNA326046 | DNA326047 | DNA326058 | DNA326059 |
| DNA326065 | DNA326067 | DNA326074 | DNA326075 | DNA326099 | DNA326104 |
| DNA326105 | DNA326121 | DNA326122 | DNA326123 | DNA326124 | DNA326126 |
| DNA326128 | DNA326129 | DNA326131 | DNA326133 | DNA326136 | DNA326137 |
| DNA326143 | DNA326147 | DNA326148 | DNA274002 | DNA326156 | DNA326157 |
| DNA194805 | DNA326180 | DNA326183 | DNA326186 | DNA326193 | DNA326195 |
| DNA326196 | DNA326197 | DNA326199 | DNA326216 | DNA326235 | DNA326236 |
| DNA326263 | DNA97300 | DNA297388 | DNA326278 | DNA326279 | DNA326288 |
| DNA326289 | DNA326292 | DNA326293 | DNA326294 | DNA227084 | DNA326296 |
| DNA326298 | DNA326299 | DNA326301 | DNA326304 | DNA326305 | DNA326306 |
| DNA326309 | DNA326310 | DNA326311 | DNA326316 | DNA326317 | DNA270979 |
| DNA326328 | DNA326333 | DNA326338 | DNA326343 | DNA326349 | DNA326351 |
| DNA326356 | DNA326362 | DNA270901 | DNA326374 | DNA326375 | DNA326378 |
| DNA326381 | DNA326397 | DNA326406 | DNA326411 | DNA129504 | DNA326416 |
| DNA326420 | DNA326423 | DNA326426 | DNA326427 | DNA326430 | DNA326443 |
| DNA326444 | DNA326449 | DNA326450 | DNA326451 | DNA326452 | DNA326453 |
| DNA326454 | DNA326457 | DNA326460 | DNA326463 | DNA326469 | DNA326487 |
| DNA326500 | DNA326501 | DNA326503 | DNA326504 | DNA326512 | DNA326533 |
| DNA326539 | DNA326548 | DNA326550 | DNA326556 | DNA326558 | DNA326566 |
| DNA326568 | DNA326573 | DNA326577 | DNA326578 | DNA326579 | DNA326586 |
| DNA326595 | DNA326596 | DNA326599 | DNA326603 | DNA269630 | DNA326607 |
| DNA326614 | DNA326621 | DNA326625 | DNA326629 | DNA326630 | DNA326633 |
| DNA326634 | DNA326648 | DNA326651 | DNA326652 | DNA273474 | DNA326671 |
| DNA326676 | DNA326680 | DNA326691 | DNA326693 | DNA326695 | DNA326698 |
| DNA32670 | DNA326703 | DNA326704 | DNA326705 | DNA326706 | DNA326707 |
| DNA326708 | DNA326709 | DNA257531 | DNA256533 | DNA326717 | DNA326718 |
| DNA326725 | DNA290260 | DNA326740 | DNA326745 | DNA326749 | DNA326752 |
| DNA326756 | DNA326758 | DNA273346 | DNA326764 | DNA297288 | DNA287270 |
| DNA326789 | DNA326790 | DNA326796 | DNA326800 | DNA326805 | DNA326808 |
| DNA326809 | DNA326810 | DNA326811 | DNA326818 | DNA326819 | DNA326821 |
| DNA194701 | DNA326829 | DNA326831 | DNA103525 | DNA326838 | DNA326841 |
| DNA88239 | DNA326845 | DNA326850 | DNA326851 | DNA269526 | DNA326868 |
| DNA326874 | DNA326875 | DNA326876 | DNA326879 | DNA326882 | DNA326884 |
| DNA326886 | DNA188732 | DNA254572 | DNA326890 | DNA151898 | DNA326894 |
| DNA326898 | DNA326901 | DNA326904 | DNA226409 | DNA326906 | DNA326909 |
| DNA326915 | DNA326921 | DNA326925 | DNA226561 | DNA326926 | DNA326927 |
| DNA326936 | DNA326937 | DNA326941 | DNA269830 | DNA326946 | DNA326952 |
| DNA326953 | DNA326954 | DNA326956 | DNA326958 | DNA188740 | DNA326960 |
| DNA254240 | DNA326974 | DNA326977 | DNA326979 | DNA326981 | DNA326982 |
| DNA326989 | DNA326990 | DNA237931 | DNA326998 | DNA327001 | DNA327003 |
| DNA327005 | DNA327008 | DNA327013 | DNA327023 | DNA327025 | DNA327029 |
| DNA327031 | DNA327033 | DNA327041 | DNA227943 | DNA327051 | DNA327058 |
| DNA327060 | DNA327067 | DNA327068 | DNA270496 | DNA327077 | DNA327078 |
| DNA327079 | DNA327086 | DNA327089 | DNA327093 | DNA327099 | DNA327102 |
| DNA327104 | DNA227013 | DNA327120 | DNA327122 | DNA327124 | DNA327125 |

ENDOCRINE

| | | | | | |
|---|---|---|---|---|---|
| DNA323772 | DNA323943 | DNA323976 | DNA254298 | DNA324100 | DNA227528 |
| DNA324139 | DNA324285 | DNA79129 | DNA324484 | DNA290585 | DNA324550 |
| DNA324642 | DNA324692 | DNA324910 | DNA324964 | DNA325350 | DNA325549 |
| DNA325615 | DNA325884 | DNA325916 | DNA325991 | DNA326003 | DNA188351 |
| DNA326328 | DNA326619 | DNA304658 | DNA326790 | DNA83170 | |

KIDNEY

| | | | | | |
|---|---|---|---|---|---|
| DNA287173 | DNA103253 | DNA323858 | DNA323859 | DNA323869 | DNA323871 |
| DNA323872 | DNA323927 | DNA323947 | DNA226619 | DNA323964 | DNA324042 |
| DNA324048 | DNA324063 | DNA324090 | DNA324092 | DNA324111 | DNA324112 |
| DNA324193 | DNA324210 | DNA324218 | DNA324294 | DNA226547 | DNA324338 |
| DNA324340 | DNA324341 | DNA324347 | DNA324398 | DNA324417 | DNA324418 |
| DNA324424 | DNA324426 | DNA324427 | DNA324434 | DNA324437 | DNA324472 |
| DNA324521 | DNA324525 | DNA324561 | DNA324595 | DNA324604 | DNA324613 |
| DNA83020 | DNA324639 | DNA324641 | DNA324645 | DNA324685 | DNA324715 |
| DNA324716 | DNA324717 | DNA324720 | DNA324722 | DNA324727 | DNA304680 |
| DNA324737 | DNA324751 | DNA304661 | DNA324790 | DNA324798 | DNA324830 |
| DNA324844 | DNA225631 | DNA274326 | DNA324922 | DNA324926 | DNA304710 |
| DNA324963 | DNA324989 | DNA324998 | DNA325026 | DNA325028 | DNA325104 |
| DNA325105 | DNA325106 | DNA325111 | DNA325126 | DNA325152 | DNA325153 |
| DNA325182 | DNA325184 | DNA325222 | DNA325296 | DNA325303 | DNA325326 |
| DNA325334 | DNA325347 | DNA325360 | DNA325384 | DNA325389 | DNA325414 |
| DNA325446 | DNA325475 | DNA325523 | DNA325535 | DNA325601 | DNA225632 |
| DNA325633 | DNA325642 | DNA325644 | DNA270458 | DNA325731 | DNA325750 |
| DNA325752 | DNA325758 | DNA325786 | DNA302016 | DNA325789 | DNA325804 |
| DNA325809 | DNA325810 | DNA325811 | DNA325812 | DNA281436 | DNA325935 |
| DNA325952 | DNA325985 | DNA326002 | DNA326003 | DNA326022 | DNA287331 |
| DNA326041 | DNA326046 | DNA326047 | DNA326099 | DNA326233 | DNA326234 |
| DNA326237 | DNA97300 | DNA326291 | DNA326292 | DNA326311 | DNA326370 |
| DNA326397 | DNA326422 | DNA326463 | DNA326469 | DNA326559 | DNA326586 |
| DNA326603 | DNA326633 | DNA326634 | DNA326692 | DNA326769 | DNA287270 |
| DNA326884 | DNA326885 | DNA326886 | DNA326952 | DNA326974 | DNA327023 |
| DNA327025 | DNA327029 | DNA327067 | DNA327085 | DNA327116 | |

LUNG

| | | | | | |
|---|---|---|---|---|---|
| DNA323717 | DNA323718 | DNA323719 | DNA287173 | DNA323740 | DNA226262 |
| DNA323778 | DNA323783 | DNA274745 | DNA323829 | DNA323832 | DNA323839 |
| DNA323841 | DNA323856 | DNA323858 | DNA323859 | DNA323862 | DNA323863 |
| DNA323864 | DNA323865 | DNA323866 | DNA323867 | DNA323871 | DNA323872 |
| DNA323878 | DNA323887 | DNA323892 | DNA227529 | DNA323902 | DNA290284 |
| DNA323910 | DNA304666 | DNA304720 | DNA323922 | DNA323925 | DNA323927 |
| DNA323936 | DNA226793 | DNA323944 | DNA323945 | DNA323947 | DNA323954 |
| DNA323959 | DNA323964 | DNA323965 | DNA323995 | DNA324005 | DNA324006 |
| DNA324020 | DNA324021 | DNA324033 | DNA324036 | DNA324039 | DNA324040 |
| DNA324041 | DNA324042 | DNA324044 | DNA324047 | DNA324048 | DNA324049 |
| DNA324052 | DNA324054 | DNA324060 | DNA324063 | DNA324067 | DNA324073 |
| DNA324090 | DNA324091 | DNA324092 | DNA324094 | DNA324101 | DNA324105 |
| DNA324109 | DNA324111 | DNA324112 | DNA227795 | DNA324134 | DNA324148 |
| DNA324155 | DNA324170 | DNA324182 | DNA324203 | DNA324204 | DNA324207 |
| DNA324210 | DNA324218 | DNA324232 | DNA324261 | DNA324265 | DNA324273 |
| DNA324293 | DNA324294 | DNA226547 | DNA324295 | DNA324320 | DNA324326 |
| DNA324338 | DNA324339 | DNA324340 | DNA324341 | DNA324358 | DNA324365 |
| DNA324380 | DNA324412 | DNA324414 | DNA324416 | DNA324417 | DNA324418 |
| DNA324434 | DNA324436 | DNA324437 | DNA324444 | DNA324453 | DNA324454 |
| DNA324472 | DNA324475 | DNA324483 | DNA324491 | DNA290585 | DNA324502 |
| DNA324504 | DNA324505 | DNA324510 | DNA324515 | DNA324521 | DNA324525 |
| DNA324541 | DNA324549 | DNA324552 | DNA324557 | DNA324558 | DNA324561 |
| DNA324564 | DNA324579 | DNA324584 | DNA324591 | DNA324592 | DNA324596 |
| DNA324597 | DNA324598 | DNA324599 | DNA324600 | DNA324604 | DNA324613 |
| DNA324633 | DNA324641 | DNA324643 | DNA324685 | DNA324697 | DNA324699 |
| DNA324700 | DNA324702 | DNA324703 | DNA324707 | DNA324714 | DNA324715 |
| DNA324716 | DNA324717 | DNA324720 | DNA304680 | DNA324736 | DNA324737 |
| DNA324745 | DNA324749 | DNA324751 | DNA324755 | DNA324756 | DNA227442 |
| DNA324771 | DNA324784 | DNA324785 | DNA324790 | DNA324796 | DNA324797 |
| DNA324803 | DNA290785 | DNA324814 | DNA324815 | DNA324816 | DNA324819 |
| DNA324828 | DNA324829 | DNA324841 | DNA324844 | DNA324846 | DNA271418 |
| DNA324870 | DNA324873 | DNA324874 | DNA324875 | DNA324884 | DNA324885 |
| DNA324887 | DNA324888 | DNA324889 | DNA274326 | DNA324896 | DNA324900 |
| DNA324904 | DNA324906 | DNA324907 | DNA324908 | DNA275334 | DNA324925 |
| DNA324926 | DNA273865 | DNA103588 | DNA324945 | DNA324946 | DNA324956 |
| DNA324961 | DNA304710 | DNA324962 | DNA324963 | DNA324965 | DNA324966 |
| DNA324967 | DNA324968 | DNA324982 | DNA324983 | DNA272090 | DNA324989 |
| DNA325002 | DNA325015 | DNA325016 | DNA325017 | DNA325024 | DNA325026 |
| DNA325027 | DNA325029 | DNA325033 | DNA325034 | DNA325039 | DNA325055 |
| DNA325056 | DNA325057 | DNA325078 | DNA325079 | DNA325080 | DNA325081 |
| DNA325100 | DNA325104 | DNA325105 | DNA325106 | DNA226496 | DNA325116 |
| DNA325117 | DNA325118 | DNA325119 | DNA325128 | DNA325141 | DNA325146 |
| DNA325152 | DNA325153 | DNA325156 | DNA325157 | DNA226345 | DNA325173 |
| DNA290319 | DNA325182 | DNA325183 | DNA325184 | DNA325190 | DNA325196 |
| DNA325209 | DNA325214 | DNA325217 | DNA325222 | DNA325233 | DNA325235 |
| DNA325236 | DNA325246 | DNA325247 | DNA325250 | DNA325278 | DNA325284 |
| DNA325285 | DNA325286 | DNA325303 | DNA325305 | DNA325326 | DNA325334 |
| DNA304459 | DNA325343 | DNA325344 | DNA325347 | DNA325353 | DNA325358 |
| DNA325360 | DNA325379 | DNA325384 | DNA325389 | DNA325401 | DNA325414 |
| DNA325418 | DNA325441 | DNA325451 | DNA325452 | DNA325456 | DNA325463 |
| DNA325475 | DNA325479 | DNA325483 | DNA325502 | DNA325506 | DNA325509 |
| DNA325510 | DNA325516 | DNA325522 | DNA325523 | DNA325527 | DNA325534 |
| DNA325535 | DNA325550 | DNA325569 | DNA325570 | DNA325584 | DNA325593 |
| DNA325595 | DNA151827 | DNA325601 | DNA225632 | DNA103514 | DNA325604 |
| DNA325618 | DNA325625 | DNA325633 | DNA325634 | DNA271344 | DNA325642 |
| DNA325644 | DNA325645 | DNA325658 | DNA325659 | DNA325660 | DNA325662 |
| DNA270458 | DNA227092 | DNA325674 | DNA325680 | DNA325686 | DNA325695 |
| DNA325704 | DNA325711 | DNA325712 | DNA325720 | DNA325731 | DNA325750 |
| DNA325752 | DNA325755 | DNA325757 | DNA325758 | DNA325773 | DNA325775 |
| DNA325776 | DNA325786 | DNA302016 | DNA325789 | DNA325806 | DNA325809 |
| DNA325810 | DNA325811 | DNA325812 | DNA325814 | DNA325818 | DNA325822 |
| DNA325837 | DNA325838 | DNA325843 | DNA325844 | DNA325864 | DNA325891 |
| DNA325894 | DNA325913 | DNA325920 | DNA269498 | DNA325923 | DNA325933 |
| DNA325935 | DNA325945 | DNA103509 | DNA325952 | DNA325953 | DNA325957 |
| DNA325958 | DNA325965 | DNA325985 | DNA325988 | DNA325994 | DNA326002 |
| DNA226646 | DNA326022 | DNA287331 | DNA326041 | DNA326046 | DNA326047 |
| DNA326099 | DNA326102 | DNA326116 | DNA326121 | DNA326122 | DNA326124 |
| DNA326128 | DNA326129 | DNA326133 | DNA289522 | DNA326136 | DNA326146 |
| DNA326155 | DNA326156 | DNA326168 | DNA326169 | DNA287355 | DNA326177 |
| DNA326186 | DNA326194 | DNA326214 | DNA326230 | DNA326233 | DNA326234 |
| DNA326256 | DNA326260 | DNA97300 | DNA326273 | DNA326278 | DNA326279 |
| DNA326287 | DNA326288 | DNA326289 | DNA326291 | DNA326292 | DNA326296 |
| DNA326297 | DNA326300 | DNA326309 | DNA326311 | DNA326330 | DNA272889 |
| DNA270975 | DNA326347 | DNA270901 | DNA326381 | DNA326384 | DNA326396 |
| DNA326404 | DNA129504 | DNA326414 | DNA326415 | DNA326416 | DNA326426 |
| DNA326427 | DNA326429 | DNA326430 | DNA326432 | DNA326433 | DNA326440 |
| DNA326441 | DNA326442 | DNA326446 | DNA326449 | DNA326450 | DNA326451 |
| DNA326452 | DNA326453 | DNA326454 | DNA271841 | DNA326457 | DNA326459 |
| DNA326463 | DNA326479 | DNA326481 | DNA326482 | DNA326484 | DNA326485 |
| DNA326487 | DNA326499 | DNA326512 | DNA287636 | DNA326516 | DNA326523 |
| DNA326559 | DNA326562 | DNA326573 | DNA326579 | DNA326581 | DNA326582 |
| DNA326583 | DNA326584 | DNA326585 | DNA274034 | DNA326596 | DNA326597 |
| DNA326603 | DNA326615 | DNA326625 | DNA326626 | DNA326633 | DNA326634 |
| DNA326642 | DNA326651 | DNA326657 | DNA326660 | DNA326661 | DNA274139 |
| DNA326676 | DNA326683 | DNA326684 | DNA326685 | DNA326687 | DNA326688 |
| DNA326690 | DNA326691 | DNA326692 | DNA326698 | DNA326702 | DNA103580 |
| DNA326726 | DNA326727 | DNA326731 | DNA290260 | DNA326736 | DNA326739 |
| DNA326741 | DNA326742 | DNA326756 | DNA326758 | DNA326761 | DNA273346 |
| DNA254548 | DNA326769 | DNA326773 | DNA287270 | DNA326781 | DNA326782 |
| DNA326787 | DNA326789 | DNA326798 | DNA326801 | DNA326808 | DNA326818 |
| DNA326819 | DNA273517 | DNA194701 | DNA103525 | DNA326844 | DNA326884 |
| DNA326885 | DNA326886 | DNA254572 | DNA326901 | DNA326902 | DNA326921 |
| DNA326937 | DNA269830 | DNA326952 | DNA326953 | DNA326972 | DNA326974 |
| DNA326981 | DNA326983 | DNA327005 | DNA327023 | DNA327025 | DNA327029 |
| DNA327033 | DNA327054 | DNA327060 | DNA327067 | DNA327068 | DNA327077 |
| DNA327078 | DNA327079 | DNA327085 | DNA327111 | DNA227013 | |

BREAST

| | | | | | |
|---|---|---|---|---|---|
| DNA323717 | DNA273712 | DNA226262 | DNA323778 | DNA323784 | DNA323804 |
| DNA323805 | DNA323817 | DNA323820 | DNA323829 | DNA323836 | DNA323845 |
| DNA323858 | DNA323859 | DNA323862 | DNA323863 | DNA323867 | DNA323868 |
| DNA323869 | DNA323870 | DNA323871 | DNA323872 | DNA323919 | DNA323922 |
| DNA323936 | DNA323943 | DNA323944 | DNA323947 | DNA323953 | DNA323964 |
| DNA323980 | DNA323990 | DNA323998 | DNA324004 | DNA324009 | DNA324013 |
| DNA324042 | DNA324047 | DNA324054 | DNA324063 | DNA324075 | DNA324090 |
| DNA324091 | DNA324092 | DNA324101 | DNA324103 | DNA324110 | DNA324111 |
| DNA324112 | DNA227795 | DNA324134 | DNA227190 | DNA324149 | DNA324154 |
| DNA324159 | DNA324170 | DNA324178 | DNA324189 | DNA324192 | DNA324193 |
| DNA324207 | DNA324210 | DNA324218 | DNA324224 | DNA324230 | DNA324236 |
| DNA324243 | DNA324276 | DNA324285 | DNA226547 | DNA324295 | DNA150976 |
| DNA324320 | DNA324338 | DNA324340 | DNA324341 | DNA324346 | DNA324347 |
| DNA324373 | DNA324390 | DNA324391 | DNA324394 | DNA324412 | DNA324417 |
| DNA324418 | DNA324423 | DNA324434 | DNA324437 | DNA324438 | DNA139747 |
| DNA325837 | DNA325838 | DNA325839 | DNA325843 | DNA325844 | DNA325848 |
| DNA325900 | DNA325906 | DNA325907 | DNA325908 | DNA325913 | DNA325922 |
| DNA325930 | DNA325933 | DNA325935 | DNA325966 | DNA227559 | DNA325985 |
| DNA325986 | DNA227206 | DNA325990 | DNA325991 | DNA219233 | DNA325994 |
| DNA325998 | DNA326000 | DNA326002 | DNA326022 | DNA326041 | DNA326046 |
| DNA326047 | DNA326075 | DNA326079 | DNA326099 | DNA326113 | DNA326115 |
| DNA97293 | DNA326122 | DNA326124 | DNA326128 | DNA326129 | DNA326136 |
| DNA326156 | DNA287355 | DNA326187 | DNA326233 | DNA326234 | DNA326251 |
| DNA326254 | DNA326260 | DNA97300 | DNA326273 | DNA326278 | DNA326280 |
| DNA326281 | DNA304715 | DNA326282 | DNA326286 | DNA290292 | DNA326289 |
| DNA326291 | DNA326292 | DNA66475 | DNA326324 | DNA326326 | DNA326327 |
| DNA326364 | DNA326378 | DNA326381 | DNA326396 | DNA326415 | DNA326449 |
| DNA326450 | DNA326451 | DNA326452 | DNA326453 | DNA326454 | DNA326457 |
| DNA326463 | DNA326469 | DNA326499 | DNA287636 | DNA326529 | DNA326541 |
| DNA270315 | DNA326546 | DNA326557 | DNA326559 | DNA326562 | DNA326579 |
| DNA326615 | DNA326620 | DNA227249 | DNA326633 | DNA326634 | DNA326635 |
| DNA326651 | DNA326657 | DNA272347 | DNA326669 | DNA326686 | DNA326687 |
| DNA326688 | DNA326698 | DNA326732 | DNA290260 | DNA326741 | DNA326742 |
| DNA83154 | DNA326756 | DNA326758 | DNA326759 | DNA326769 | DNA326777 |
| DNA287270 | DNA326792 | DNA326796 | DNA326798 | DNA326799 | DNA326816 |
| DNA194701 | DNA103525 | DNA326841 | DNA326862 | DNA326863 | DNA304670 |
| DNA326864 | DNA326866 | DNA326870 | DNA326885 | DNA326886 | DNA326903 |
| DNA326921 | DNA326952 | DNA326969 | DNA326971 | DNA326974 | DNA326981 |
| DNA327016 | DNA327023 | DNA327025 | DNA327029 | DNA273992 | DNA327060 |
| DNA327062 | DNA273254 | DNA327067 | DNA327068 | DNA327073 | DNA327085 |
| DNA327087 | DNA327090 | DNA327092 | DNA276159 | DNA327127 | |

STOMACH

| | | | | | |
|---|---|---|---|---|---|
| DNA287173 | DNA323805 | DNA323849 | DNA323864 | DNA323865 | DNA323866 |
| DNA323873 | DNA323884 | DNA323920 | DNA323925 | DNA323934 | DNA323990 |
| DNA324028 | DNA324029 | DNA324039 | DNA324048 | DNA324065 | DNA227545 |
| DNA227795 | DNA324155 | DNA324179 | DNA324180 | DNA324216 | DNA324243 |
| DNA324244 | DNA324294 | DNA324362 | DNA324364 | DNA324398 | DNA324417 |
| DNA324418 | DNA324471 | DNA324504 | DNA324541 | DNA324552 | DNA324555 |
| DNA324556 | DNA324558 | DNA324624 | DNA324630 | DNA304680 | DNA324756 |
| DNA324769 | DNA324790 | DNA324808 | DNA324850 | DNA225631 | DNA324906 |
| DNA324907 | DNA324908 | DNA324922 | DNA304710 | DNA324962 | DNA324963 |
| DNA324972 | DNA324973 | DNA324982 | DNA324997 | DNA325033 | DNA325074 |
| DNA325078 | DNA325079 | DNA325104 | DNA325105 | DNA325106 | DNA325148 |
| DNA325149 | DNA325156 | DNA325157 | DNA89242 | DNA325186 | DNA325191 |
| DNA325192 | DNA325202 | DNA325224 | DNA325233 | DNA325235 | DNA325236 |
| DNA325251 | DNA325262 | DNA325268 | DNA325306 | DNA325316 | DNA325318 |
| DNA325320 | DNA325368 | DNA325418 | DNA97285 | DNA325441 | DNA325442 |
| DNA325444 | DNA325446 | DNA325474 | DNA325480 | DNA325506 | DNA325534 |
| DNA325535 | DNA325570 | DNA325601 | DNA225632 | DNA325642 | DNA325644 |
| DNA325645 | DNA270458 | DNA227092 | DNA325773 | DNA325775 | DNA325776 |
| DNA325803 | DNA325804 | DNA274058 | DNA325843 | DNA325873 | DNA325941 |
| DNA325986 | DNA325993 | DNA326019 | DNA287331 | DNA326043 | DNA326133 |
| DNA326196 | DNA326284 | DNA326311 | DNA326333 | DNA326347 | DNA326397 |
| DNA326427 | DNA326517 | DNA326603 | DNA326641 | DNA326642 | DNA326698 |
| DNA326750 | DNA326791 | DNA326846 | DNA326859 | DNA326862 | DNA326863 |
| DNA304670 | DNA326864 | DNA326865 | DNA326918 | DNA326961 | DNA326977 |
| DNA326983 | DNA327040 | DNA327042 | DNA327055 | DNA273254 | DNA327099 |
| DNA327116 | DNA327127 | | | | |

BONE

| | | | | | |
|---|---|---|---|---|---|
| DNA323765 | DNA323817 | DNA323820 | DNA323829 | DNA323864 | DNA323867 |
| DNA323869 | DNA323871 | DNA323914 | DNA323947 | DNA323964 | DNA324004 |
| DNA324009 | DNA324090 | DNA324091 | DNA324092 | DNA324111 | DNA324112 |
| DNA324154 | DNA324155 | DNA324200 | DNA324201 | DNA324210 | DNA324230 |
| DNA324293 | DNA226547 | DNA324295 | DNA324326 | DNA324347 | DNA324390 |
| DNA324417 | DNA324418 | DNA324423 | DNA324437 | DNA324472 | DNA324483 |
| DNA324488 | DNA324501 | DNA324502 | DNA324503 | DNA324504 | DNA324505 |
| DNA324512 | DNA324521 | DNA324525 | DNA324541 | DNA324549 | DNA324550 |
| DNA324551 | DNA324554 | DNA324555 | DNA324556 | DNA324557 | DNA324558 |
| DNA324575 | DNA324576 | DNA324579 | DNA324595 | DNA324596 | DNA324604 |
| DNA324613 | DNA324624 | DNA324632 | DNA324641 | DNA324645 | DNA324682 |
| DNA324687 | DNA324697 | DNA324717 | DNA324720 | DNA324737 | DNA324756 |
| DNA304661 | DNA324785 | DNA324796 | DNA324797 | DNA150772 | DNA324828 |
| DNA324829 | DNA324844 | DNA324866 | DNA324902 | DNA324904 | DNA324905 |
| DNA324906 | DNA324926 | DNA324989 | DNA325015 | DNA325024 | DNA325026 |
| DNA325027 | DNA325034 | DNA325111 | DNA325116 | DNA131588 | DNA325156 |
| DNA325157 | DNA325164 | DNA325179 | DNA325182 | DNA325183 | DNA325184 |
| DNA325202 | DNA325206 | DNA325222 | DNA325229 | DNA325231 | DNA325232 |
| DNA325234 | DNA325236 | DNA325250 | DNA325301 | DNA325303 | DNA325326 |
| DNA325339 | DNA325340 | DNA325347 | DNA325358 | DNA325395 | DNA325430 |
| DNA325437 | DNA325451 | DNA325452 | DNA325523 | DNA325558 | DNA325570 |
| DNA325576 | DNA325601 | DNA225632 | DNA325633 | DNA325731 | DNA325733 |
| DNA325736 | DNA325762 | DNA325786 | DNA302016 | DNA325789 | DNA325806 |
| DNA325810 | DNA325811 | DNA325812 | DNA325843 | DNA325844 | DNA325906 |
| DNA325908 | DNA325913 | DNA325922 | DNA325935 | DNA325985 | DNA326002 |
| DNA326041 | DNA326046 | DNA326099 | DNA326233 | DNA326234 | DNA326251 |
| DNA97300 | DNA304715 | DNA326286 | DNA326289 | DNA326381 | DNA326457 |
| DNA326580 | DNA326633 | DNA326634 | DNA326635 | DNA326651 | DNA290260 |
| DNA326796 | DNA326884 | DNA326886 | DNA326974 | DNA326977 | DNA327005 |
| DNA327025 | DNA327060 | DNA327062 | DNA327067 | DNA327114 | |

### Example 2

### Use of TAT as a Hybridization Probe

The following method describes use of a nucleotide sequence encoding TAT as a hybridization probe for, i.e., diagnosis of the presence of a tumor in a mammal.

DNA comprising the coding sequence of full-length or mature TAT as disclosed herein can also be employed as a probe to screen for homologous DNAs (such as those encoding naturally-occurring variants of TAT) in human tissue cDNA libraries or human tissue genomic libraries.

Hybridization and washing of filters containing either library DNAs is performed under the following high stringency conditions. Hybridization of radiolabeled TAT-derived probe to the filters is performed in a solution of 50% formamide, 5×SSC, 0.1% SDS, 0.1% sodium pyrophosphate, 50 mM sodium phosphate, pH 6.8, 2× Denhardt's solution, and 10% dextran sulfate at 42° C. for 20 hours. Washing of the filters is performed in an aqueous solution of 0.1×SSC and 0.1% SDS at 42° C.

DNAs having a desired sequence identity with the DNA encoding full-length native sequence TAT can then be identified using standard techniques known in the art.

### Example 3

### Expression of TAT in E. coli

This example illustrates preparation of an unglycosylated form of TAT by recombinant expression in E. coli.

The DNA sequence encoding TAT is initially amplified using selected PCR primers. The primers should contain restriction enzyme sites which correspond to the restriction enzyme sites on the selected expression vector. A variety of expression vectors may be employed. An example of a suitable vector is pBR322 (derived from E. coli; see Bolivar et al., Gene, 2:95 (1977)) which contains genes for ampicillin and tetracycline resistance. The vector is digested with restriction enzyme and dephosphorylated. The PCR amplified sequences are then ligated into the vector. The vector will preferably include sequences which encode for an antibiotic resistance gene, a trp promoter, a polyhis leader (including the first six STII codons, polyhis sequence, and enterokinase cleavage site), the TAT coding region, lambda transcriptional terminator, and an argu gene.

The ligation mixture is then used to transform a selected E. coli strain using the methods described in Sambrook et al., sulra. Transformants are identified by their ability to grow on LB plates and antibiotic resistant colonies are then selected. Plasmid DNA can be isolated and confirmed by restriction analysis and DNA sequencing.

Selected clones can be grown overnight in liquid culture medium such as LB broth supplemented with antibiotics. The overnight culture may subsequently be used to inoculate a larger scale culture. The cells are then grown to a desired optical density, during which the expression promoter is turned on.

After culturing the cells for several more hours, the cells can be harvested by centrifugation. The cell pellet obtained by the centrifugation can be solubilized using various agents known in the art, and the solubilized TAT protein can then be purified using a metal chelating column under conditions that allow tight binding of the protein.

TAT may be expressed in E. coli in a poly-His tagged form, using the following procedure. The DNA encoding TAT is initially amplified using selected PCR primers. The primers will contain restriction enzyme sites which correspond to the restriction enzyme sites on the selected expression vector, and other useful sequences providing for efficient and reliable translation initiation, rapid purification on a metal chelation column, and proteolytic removal with enterokinase. The PCR-amplified, poly-His tagged sequences are then ligated into an expression vector, which is used to transform an E. coli host based on strain 52 (W3110 fuhA(tonA) Ion gale rpoHts(htpRts) clpP(lacIq). Transformants are first grown in LB containing 50 mg/ml carbenicillin at 30° C. with shaking until an O.D.600 of 3-5 is reached. Cultures are then diluted 50-100 fold into CRAP media (prepared by mixing 3.57 g (NH₄)₂SO₄, 0.71 g sodium citrate·2H₂O, 1.07 g KCl, 5.36 g Difco yeast extract, 5.36 g Sheffield hycase SF in 500 mL water, as well as 110 mM MPOS, pH 7.3, 0.55% (w/v) glucose and 7 mM MgSO₄) and grown for approximately 20-30 hours at 30° C. with shaking. Samples are removed to verify expression by SDS-PAGE analysis, and the bulk culture is centrifuged to pellet the cells. Cell pellets are frozen until purification and refolding.

E. coli paste from 0.5 to 1 L fermentations (6-10 g pellets) is resuspended in 10 volumes (w/v) in 7 M guanidine, 20 mM Tris, pH 8 buffer. Solid sodium sulfite and sodium tetrathionate is added to make final concentrations of 0.1M and 0.02 M, respectively, and the solution is stirred overnight at 4° C. This step results in a denatured protein with all cysteine residues blocked by sulfitolization. The solution is centrifuged at 40,000 rpm in a Beckman Ultracentifuge for 30 min. The supernatant is diluted with 3-5 volumes of metal chelate column buffer (6 M guanidine, 20 mM Tris, pH 7.4) and filtered through 0.22 micron filters to clarify. The clarified extract is loaded onto a 5 ml Qiagen Ni-NTA metal chelate column equilibrated in the metal chelate column buffer. The column is washed with additional buffer containing 50 mM imidazole (Calbiochem, Utrol grade), pH 7.4. The protein is eluted with buffer containing 250 mM imidazole. Fractions containing the desired protein are pooled and stored at 4° C. Protein concentration is estimated by its absorbance at 280 nm using the calculated extinction coefficient based on its amino acid sequence.

The proteins are refolded by diluting the sample slowly into freshly prepared refolding buffer consisting of: 20 mM Tris, pH 8.6, 0.3 M NaCl, 2.5 M urea, 5 mM cysteine, 20 mM glycine and 1 mM EDTA. Refolding volumes are chosen so that the final protein concentration is between 50 to 100 micrograms/ml. The refolding solution is stirred gently at 4° C. for 12-36 hours. The refolding reaction is quenched by the addition of TFA to a final concentration of 0.4% (pH of approximately 3). Before further purification of the protein, the solution is filtered through a 0.22 micron filter and acetonitrile is added to 2-10% final concentration. The refolded protein is chromatographed on a Poros R1/H reversed phase column using a mobile buffer of 0.1% TFA with elution with a gradient of acetonitrile from 10 to 80%. Aliquots of fractions with A280 absorbance are analyzed on SDS polyacrylamide gels and fractions containing homogeneous refolded protein are pooled. Generally, the properly refolded species of most proteins are eluted at the lowest concentrations of acetonitrile since those species are the most compact with their hydrophobic interiors shielded from interaction with the reversed phase resin. Aggregated species are usually eluted at higher acetonitrile concentrations. In addition to resolving misfolded forms of proteins from the desired form, the reversed phase step also removes endotoxin from the samples.

Fractions containing the desired folded TAT polypeptide are pooled and the acetonitrile removed using a gentle stream of nitrogen directed at the solution. Proteins are formulated into 20 mM Hepes, pH 6.8 with 0.14 M sodium chloride and 4% mannitol by dialysis or by gel filtration using G25 Superfine (Pharmacia) resins equilibrated in the formulation buffer and sterile filtered.

Certain of the TAT polypeptides disclosed herein have been successfully expressed and purified using this technique(s).

### Example 4

### Expression of TAT in Mammalian Cells

This example illustrates preparation of a potentially glycosylated form of TAT by recombinant expression in mammalian cells.

The vector, pRK5 (see EP 307,247, published Mar. 15, 1989), is employed as the expression vector. Optionally, the TAT DNA is ligated into pRK5 with selected restriction enzymes to allow insertion of the TAT DNA using ligation methods such as described in Sambrook et al., supra. The resulting vector is called pRK5-TAT.

In one embodiment, the selected host cells may be 293 cells. Human 293 cells (ATCC CCL 1573) are grown to confluence in tissue culture plates in medium such as DMEM supplemented with fetal calf serum and optionally, nutrient components and/or antibiotics. About 10 μg pRK5-TAT DNA is mixed with about 1 μg DNA encoding the VA RNA gene [Thimmappaya et al., Cell 31:543 (1982)] and dissolved in 500 μl of 1 mM Tris-HCl, 0.1 mM EDTA, 0.227 M CaCl₂. To this mixture is added, dropwise, 500 μl of 50 mM HEPES (pH 7.35), 280 mM NaCl, 1.5 mM NapO₄, and a precipitate is allowed to form for 10 minutes at 25° C. The precipitate is suspended and added to the 293 cells and allowed to settle for about four hours at 37° C. The culture medium is aspirated off and 2 ml of 20% glycerol in PBS is added for 30 seconds. The 293 cells are then washed with serum free medium, fresh medium is added and the cells are incubated for about 5 days.

Approximately 24 hours after the transfections, the culture medium is removed and replaced with culture medium (alone) or culture medium containing 200 μCi/ml ³⁵S-cysteine and 200 μCi/ml ³⁵S-methionine. After a 12 hour incubation, the conditioned medium is collected, concentrated on a spin filter, and loaded onto a 15% SDS gel. The processed gel may be dried and exposed to film for a selected period of time to reveal the presence of TAT polypeptide. The cultures containing transfected cells may undergo further incubation (in serum free medium) and the medium is tested in selected bioassays.

In an alternative technique, TAT may be introduced into 293 cells transiently using the dextran sulfate method described by Somparyrac et al., Proc. Natl. Acad. Sci. 12:7575 (1981). 293 cells are grown to maximal density in a spinner flask and 700 μg pRK5-TAT DNA is added. The cells are first concentrated from the spinner flask by centrifugation and washed with PBS. The DNA-dextran precipitate is incubated on the cell pellet for four hours. The cells are treated with 20% glycerol for 90 seconds, washed with tissue culture medium, and re-introduced into the spinner flask containing tissue culture medium, 5 μg/ml bovine insulin and 0.1 μg/ml bovine transferrin. After about four days, the conditioned media is centrifuged and filtered to remove cells and debris. The sample containing expressed TAT can then be concentrated and purified by any selected method, such as dialysis and/or column chromatography.

In another embodiment, TAT can be expressed in CHO cells. The pRK5-TAT can be transfected into CHO cells using known reagents such as CaPO₄ or DEAE-dextran. As described above, the cell cultures can be incubated, and the medium replaced with culture medium (alone) or medium containing a radiolabel such as ³⁵S-methionine. After determining the presence of TAT polypeptide, the culture medium may be replaced with serum free medium. Preferably, the cultures are incubated for about 6 days, and then the conditioned medium is harvested. The medium containing the expressed TAT can then be concentrated and purified by any selected method.

Epitope-tagged TAT may also be expressed in host CHO cells. The TAT may be subcloned out of the pRK5 vector. The subclone insert can undergo PCR to fuse in frame with a selected epitope tag such as a poly-his tag into a Baculovirus expression vector. The poly-his tagged TAT insert can then be subcloned into a SV40 driven vector containing a selection marker such as DHFR for selection of stable clones. Finally, the CHO cells can be transfected (as described above) with the SV40 driven vector. Labeling may be performed, as described above, to verify expression. The culture medium containing the expressed poly-His tagged TAT can then be concentrated and purified by any selected method, such as by Ni²⁺-chelate affinity chromatography.

TAT may also be expressed in CHO and/or COS cells by a transient expression procedure or in CHO cells by another stable expression procedure.

Stable expression in CHO cells is performed using the following procedure. The proteins are expressed as an IgG construct (immunoadhesin), in which the coding sequences for the soluble forms (e.g. extracellular domains) of the respective proteins are fused to an IgG1 constant region sequence containing the hinge, CH2 and CH2 domains and/or is a poly-His tagged form.

Following PCR amplification, the respective DNAs are subcloned in a CHO expression vector using standard techniques as described in Ausubel et al., Current Protocols of Molecular Biology, Unit 3.16, John Wiley and Sons (1997). CHO expression vectors are constructed to have compatible restriction sites 5′ and 3′ of the DNA of interest to allow the convenient shuttling of cDNA's. The vector used expression in CHO cells is as described in Lucas et al., Nucl. Acids Res. 24:9 (1774-1779 (1996), and uses the SV40 early promoter/enhancer to drive expression of the cDNA of interest and dihydrofolate reductase (DHFR). DHFR expression permits selection for stable maintenance of the plasmid following transfection.

Twelve micrograms of the desired plasmid DNA is introduced into approximately 10 million CHO cells using commercially available transfection reagents Superfect® (Quiagen), Dosper® or Fugene® (Boehringer Mannheim). The cells are grown as described in Lucas et al., supra. Approximately 3×10⁷ cells are frozen in an ampule for further growth and production as described below.

The ampules containing the plasmid DNA are thawed by placement into water bath and mixed by vortexing. The contents are pipetted into a centrifuge tube containing 10 mLs of media and centrifuged at 1000 rpm for 5 minutes. The supernatant is aspirated and the cells are resuspended in 10 mL of selective media (0.2 μm filtered PS20 with 5% 0.2 μm diafiltered fetal bovine serum). The cells are then aliquoted into a 100 mL spinner containing 90 mL of selective media. After 1-2 days, the cells are transferred into a 250 mL spinner filled with 150 mL selective growth medium and incubated at 37° C. After another 2-3 days, 250 mL, 500 mL and 2000 mL spinners are seeded with 3×10⁵ cells/mL. The cell media is exchanged with fresh media by centrifugation and resuspension in production medium. Although any suitable CHO media may be employed, a production medium described in U.S. Pat. No. 5,122,469, issued Jun. 16, 1992 may actually be used. A 3 L production spinner is seeded at 1.2×10⁶ cells/mL. On day 0, the cell number pH ie determined. On day 1, the spinner is sampled and sparging with filtered air is commenced. On day 2, the spinner is sampled, the temperature shifted to 33° C., and 30 mL of 500 g/L glucose and 0.6 mL of 10% antifoam (e.g., 35% polydimethylsiloxane emulsion, Dow Corning 365 Medical Grade Emulsion) taken. Throughout the production, the pH is adjusted as necessary to keep it at around 7.2. After 10 days, or until the viability dropped below 70%, the cell culture is harvested by centrifugation and filtering through a 0.22 μm filter. The filtrate was either stored at 4° C. or immediately loaded onto columns for purification.

For the poly-His tagged constructs, the proteins are purified using a Ni-NTA column (Qiagen). Before purification, imidazole is added to the conditioned media to a concentration of 5 mM. The conditioned media is pumped onto a 6 ml Ni-NTA column equilibrated in 20 mM Hepes, pH 7.4, buffer containing 0.3 M NaCl and 5 mM imidazole at a flow rate of 4-5 ml/min. at 4° C. After loading, the column is washed with additional equilibration buffer and the protein eluted with equilibration buffer containing 0.25 M imidazole. The highly purified protein is subsequently desalted into a storage buffer containing 10 mM Hepes, 0.14 M NaCl and 4% mannitol, pH 6.8, with a 25 ml G25 Superfine (Pharmacia) column and stored at −80° C.

Immunoadhesin (Fc-containing) constructs are purified from the conditioned media as follows. The conditioned medium is pumped onto a 5 ml Protein A column (Pharmacia) which had been equilibrated in 20 mM Na phosphate buffer, pH 6.8. After loading, the column is washed extensively with equilibration buffer before elution with 100 mM citric acid, pH 3.5. The eluted protein is immediately neutralized by collecting 1 ml fractions into tubes containing 275 μL of 1 M Tris buffer, pH 9. The highly purified protein is subsequently desalted into storage buffer as described above for the poly-His tagged proteins. The homogeneity is assessed by SDS polyacrylamide gels and by N-terminal amino acid sequencing by Edman degradation.

Certain of the TAT polypeptides disclosed herein have been successfully expressed and purified using this technique(s).

### Example 5

### Expression of TAT in Yeast

The following method describes recombinant expression of TAT in yeast.

First, yeast expression vectors are constructed for intracellular production or secretion of TAT from the ADH2/GAPDH promoter. DNA encoding TAT and the promoter is inserted into suitable restriction enzyme sites in the selected plasmid to direct intracellular expression of TAT. For secretion, DNA encoding TAT can be cloned into the selected plasmid, together with DNA encoding the ADH2/GAPDH promoter, a native TAT signal peptide or other mammalian signal peptide, or, for example, a yeast alpha-factor or invertase secretory signal/leader sequence, and linker sequences (if needed) for expression of TAT.

Yeast cells, such as yeast strain AB110, can then be transformed with the expression plasmids described above and cultured in selected fermentation media. The transformed yeast supernatants can be analyzed by precipitation with 10% trichloroacetic acid and separation by SDS-PAGE, followed by staining of the gels with Coomassie Blue stain.

Recombinant TAT can subsequently be isolated and purified by removing the yeast cells from the fermentation medium by centrifugation and then concentrating the medium using selected cartridge filters. The concentrate containing TAT may further be purified using selected column chromatography resins.

Certain of the TAT polypeptides disclosed herein have been successfully expressed and purified using this technique(s).

### Example 6

### Expression of TAT in Baculovirus-Infected Insect Cells

The following method describes recombinant expression of TAT in Baculovirus-infected insect cells.

The sequence coding for TAT is fused upstream of an epitope tag contained within a baculovirus expression vector. Such epitope tags include poly-his tags and immunoglobulin tags (like Fc regions of IgG). A variety of plasmids may be employed, including plasmids derived from commercially available plasmids such as pVL1393 (Novagen). Briefly, the sequence encoding TAT or the desired portion of the coding sequence of TAT such as the sequence encoding an extracellular domain of a transmembrane protein or the sequence encoding the mature protein if the protein is extracellular is amplified by PCR with primers complementary to the 5′ and 3′ regions. The 5′ primer may incorporate flanking (selected) restriction enzyme sites. The product is then digested with those selected restriction enzymes and subcloned into the expression vector.

Recombinant baculovirus is generated by co-transfecting the above plasmid and BaculoGold™ virus DNA (Pharmingen) into Spodoptera frugiperda (“Sf9”) cells (ATCC CRL 1711) using lipofectin (commercially available from GIBCO-BRL). After 4-5 days of incubation at 28° C., the released viruses are harvested and used for further amplifications. Viral infection and protein expression are performed as described by O'Reilley et al., Baculovirus expression vectors: A Laboratory Manual, Oxford: Oxford University Press (1994).

Expressed poly-his tagged TAT can then be purified, for example, by Ni²⁺-chelate affinity chromatography as follows. Extracts are prepared from recombinant virus-infected Sf9 cells as described by Rupert et al., Nature, 362:175-179 (1993). Briefly, Sf9 cells are washed, resuspended in sonication buffer (25 mL Hepes, pH 7.9; 12.5 mM MgCl₂; 0.1 mM EDTA; 10% glycerol; 0.1% NP-40; 0.4 M KCl), and sonicated twice for 20 seconds on ice. The sonicates are cleared by centrifugation, and the supernatant is diluted 50-fold in loading buffer (50 mM phosphate, 300 mM NaCl, 10% glycerol, pH 7.8) and filtered through a 0.45 μm filter. A Ni²⁺-NTA agarose column (commercially available from Qiagen) is prepared with a bed volume of 5 mL, washed with 25 mL of water and equilibrated with 25 mL of loading buffer. The filtered cell extract is loaded onto the column at 0.5 mL per minute. The column is washed to baseline A₂₈₀ with loading buffer, at which point fraction collection is started. Next, the column is washed with a secondary wash buffer (50 mM phosphate; 300 mM NaCl, 10% glycerol, pH 6.0), which elutes nonspecifically bound protein. After reaching A₂₈₀ baseline again, the column is developed with a 0 to 500 mM Imidazole gradient in the secondary wash buffer. One mL fractions are collected and analyzed by SDS-PAGE and silver staining or Western blot with Ni²⁺-NTA-conjugated to alkaline phosphatase (Qiagen). Fractions containing the eluted H₁₀-tagged TAT are pooled and dialyzed against loading buffer.

Alternatively, purification of the IgG tagged (or Fc tagged) TAT can be performed using known chromatography techniques, including for instance, Protein A or protein G column chromatography.

Certain of the TAT polypeptides disclosed herein have been successfully expressed and purified using this technique(s).

### Example 7

### Preparation of Antibodies that Bind TAT

This example illustrates preparation of monoclonal antibodies which can specifically bind TAT.

Techniques for producing the monoclonal antibodies are known in the art and are described, for instance, in Goding, supra. Immunogens that may be employed include purified TAT, fusion proteins containing TAT, and cells expressing recombinant TAT on the cell surface. Selection of the immunogen can be made by the skilled artisan without undue experimentation.

Mice, such as Balb/c, are immunized with the TAT immunogen emulsified in complete Freund's adjuvant and injected subcutaneously or intraperitoneally in an amount from 1-100 micrograms. Alternatively, the immunogen is emulsified in MPL-TDM adjuvant (Ribi Immunochemical Research, Hamilton, Mont.) and injected into the animal's hind foot pads. The immunized mice are then boosted 10 to 12 days later with additional immunogen emulsified in the selected adjuvant. Thereafter, for several weeks, the mice may also be boosted with additional immunization injections. Serum samples may be periodically obtained from the mice by retro-orbital bleeding for testing in ELISA assays to detect anti-TAT antibodies.

After a suitable antibody titer has been detected, the animals “positive” for antibodies can be injected with a final intravenous injection of TAT. Three to four days later, the mice are sacrificed and the spleen cells are harvested. The spleen cells are then fused (using 35% polyethylene glycol) to a selected murine myeloma cell line such as P3X63AgU.1, available from ATCC, No. CRL 1597. The fusions generate hybridoma cells which can then be plated in 96 well tissue culture plates containing HAT (hypoxanthine, aminopterin, and thymidine) medium to inhibit proliferation of non-fused cells, myeloma hybrids, and spleen cell hybrids.

The hybridoma cells will be screened in an ELISA for reactivity against TAT. Determination of “positive” hybridoma cells secreting the desired monoclonal antibodies against TAT is within the skill in the art.

The positive hybridoma cells can be injected intraperitoneally into syngeneic Balb/c mice to produce ascites containing the anti-TAT monoclonal antibodies. Alternatively, the hybridoma cells can be grown in tissue culture flasks or roller bottles. Purification of the monoclonal antibodies produced in the ascites can be accomplished using ammonium sulfate precipitation, followed by gel exclusion chromatography. Alternatively, affinity chromatography based upon binding of antibody to protein A or protein G can be employed.

### Example 8

### Purification of TAT Polypeptides Using Specific Antibodies

Native or recombinant TAT polypeptides may be purified by a variety of standard techniques in the art of protein purification. For example, pro-TAT polypeptide, mature TAT polypeptide, or pre-TAT polypeptide is purified by immunoaffinity chromatography using antibodies specific for the TAT polypeptide of interest. In general, an immunoaffinity column is constructed by covalently coupling the anti-TAT polypeptide antibody to an activated chromatographic resin.

Polyclonal immunoglobulins are prepared from immune sera either by precipitation with ammonium sulfate or by purification on immobilized Protein A (Pharmacia LKB Biotechnology, Piscataway, N.J.). Likewise, monoclonal antibodies are prepared from mouse ascites fluid by ammonium sulfate precipitation or chromatography on immobilized Protein A. Partially purified immunoglobulin is covalently attached to a chromatographic resin such as CnBr-activated SEPHAROSE™ (Pharmacia LKB Biotechnology). The antibody is coupled to the resin, the resin is blocked, and the derivative resin is washed according to the manufacturer's instructions.

Such an immunoaffinity column is utilized in the purification of TAT polypeptide by preparing a fraction from cells containing TAT polypeptide in a soluble form. This preparation is derived by solubilization of the whole cell or of a subcellular fraction obtained via differential centrifugation by the addition of detergent or by other methods well known in the art. Alternatively, soluble TAT polypeptide containing a signal sequence may be secreted in useful quantity into the medium in which the cells are grown.

A soluble TAT polypeptide-containing preparation is passed over the immunoaffinity column, and the column is washed under conditions that allow the preferential absorbance of TAT polypeptide (e.g., high ionic strength buffers in the presence of detergent). Then, the column is eluted under conditions that disrupt antibody/TAT polypeptide binding (e.g., a low pH buffer such as approximately pH 2-3, or a high concentration of a chaotrope such as urea or thiocyanate ion), and TAT polypeptide is collected.

### Example 9

### In Vitro Tumor Cell Killing Assay

Mammalian cells expressing the TAT polypeptide of interest may be obtained using standard expression vector and cloning techniques. Alternatively, many tumor cell lines expressing TAT polypeptides of interest are publicly available, for example, through the ATCC and can be routinely identified using standard ELISA or FACS analysis. Anti-TAT polypeptide monoclonal antibodies (and toxin conjugated derivatives thereof) may then be employed in assays to determine the ability of the antibody to kill TAT polypeptide expressing cells in vitro.

For example, cells expressing the TAT polypeptide of interest are obtained as described above and plated into 96 well dishes. In one analysis, the antibody/toxin conjugate (or naked antibody) is included throughout the cell incubation for a period of 4 days. In a second independent analysis, the cells are incubated for 1 hour with the antibody/toxin conjugate (or naked antibody) and then washed and incubated in the absence of antibody/toxin conjugate for a period of 4 days. Cell viability is then measured using the CellTiter-Glo Luminescent Cell Viability Assay from Promega (Cat# G7571). Untreated cells serve as a negative control.

### Example 10

### In Vivo Tumor Cell Killing Assay

To test the efficacy of conjugated or unconjugated anti-TAT polypeptide monoclonal antibodies, anti-TAT antibody is injected intraperitoneally into nude mice 24 hours prior to receiving tumor promoting cells subcutaneously in the flank. Antibody injections continue twice per week for the remainder of the study. Tumor volume is then measured twice per week.

The foregoing written specification is considered to be sufficient to enable one skilled in the art to practice the invention. The present invention is not to be limited in scope by the construct deposited, since the deposited embodiment is intended as a single illustration of certain aspects of the invention and any constructs that are functionally equivalent are within the scope of this invention. The deposit of material herein does not constitute an admission that the written description herein contained is inadequate to enable the practice of any aspect of the invention, including the best mode thereof, nor is it to be construed as limiting the scope of the claims to the specific illustrations that it represents. Indeed, various modifications of the invention in addition to those shown and described herein will become apparent to those skilled in the art from the foregoing description and fall within the scope of the appended claims.

1-14. (canceled)

15. An isolated antibody that binds to a polypeptide having at least 80% amino acid sequence identity to:
(a) the polypeptide shown in any one of FIGS. 1-6355 (SEQ ID NOS:1-6355);
(b) the polypeptide shown in any one of FIGS. 1-6355 (SEQ ID NOS:1-6355), lacking its associated signal peptide;
(c) an extracellular domain of the polypeptide shown in any one of FIGS. 1-6355 (SEQ ID NOS:1-6355), with its associated signal peptide;
(d) an extracellular domain of the polypeptide shown in any one of FIGS. 1-6355 (SEQ ID NOS:1-6355), lacking its associated signal peptide;
(e) a polypeptide encoded by the nucleotide sequence shown in any one of FIGS. 1-6355 (SEQ ID NOS:1-6355); or
(f) a polypeptide encoded by the full-length coding region of the nucleotide sequence shown in any one of FIGS. 1-6355 (SEQ ID NOS:1-6355).

16. An isolated antibody that binds to a polypeptide having:
(a) the amino acid sequence shown in any one of FIGS. 1-6355 (SEQ ID NOS:1-6355);
(b) the amino acid sequence shown in any one of FIGS. 1-6355 (SEQ ID NOS:1-6355), lacking its associated signal peptide sequence;
(c) an amino acid sequence of an extracellular domain of the polypeptide shown in any one of FIGS. 1-6355 (SEQ ID NOS:1-6355), with its associated signal peptide sequence;
(d) an amino acid sequence of an extracellular domain of the polypeptide shown in any one of FIGS. 1-6355 (SEQ ID NOS:1-6355), lacking its associated signal peptide sequence;
(e) an amino acid sequence encoded by the nucleotide sequence shown in any one of FIGS. 1-6355 (SEQ ID NOS:1-6355); or
(f) an amino acid sequence encoded by the full-length coding region of the nucleotide sequence shown in any one of FIGS. 1-6355 (SEQ ID NOS:1-6355).

17. The antibody of claim 15 or 16 which is a monoclonal antibody.

18. The antibody of claim 15 or 16 which is an antibody fragment.

19. The antibody of claim 15 or 16 which is a chimeric or a humanized antibody.

20. The antibody of claim 15 or 16 which is conjugated to a growth inhibitory agent.

21. The antibody of claim 15 or 16 which is conjugated to a cytotoxic agent.

22. The antibody of claim 21, wherein the cytotoxic agent is selected from the group consisting of toxins, antibiotics, radioactive isotopes and nucleolytic enzymes.

23. The antibody of claim 21, wherein the cytotoxic agent is a toxin.

24. The antibody of claim 23, wherein the toxin is selected from the group consisting of maytansinoid and calicheamicin.

25. The antibody of claim 23, wherein the toxin is a maytansinoid.

26. The antibody of claim 15 or 16 which is produced in bacteria.

27. The antibody of claim 15 or 16 which is produced in CHO cells.

28. The antibody of claim 15 or 16 which induces death of a cell to which it binds.

29. The antibody of claim 15 or 16 which is detectably labeled.

30-184. (canceled)
